SEQUENCE LISTING (258 sequences)

SEQ ID NO: 1 - DNA, 117 bp, Homo sapiens
  source 1..117: mol_type = genomic DNA
agaaacaagagggcaaggagagagcagagaacacactttgccttctctttggtattgagtaatatcaaccaaattgcagacatctcaacactttggccaggcagcctgctgagcaag

SEQ ID NO: 2 - DNA, 5168 bp, Homo sapiens
  source 1..5168: mol_type = other DNA
gtaacactcatacttttcatgccttgagccaaaatatttattacatttttatgtttctaactagaagtgcttgagctttttttccttccaggtgatgaggggatggaatgagcaaagctacatcaatttttttttaatgtatgaaaataaaaaaggtacaagaggccaagtttagggccactgaaggttcatagaaagatgcaaaatatctgaattactataaatgaatgctattgtcagaggaaaggtttaaggagtgcttcttgaatgaatgtgtacaaatcagcagaaggtaaggtgtgagactcttggaaatgaatactggtagttcaggtgagaaaaataatcaggaacataatagggtgggaggaaatgtatggtttcccaggtattaacaagtattgccaggcatttcctgaactagattggcctaagtaggagaccaatgtttctcaaaatattcactcattttagaatcactgaatgtttaaaaatgcaatttctggattccttcccaaacagccagactctttgggacctgatgatctgcatttctttttaaaaacaaactcgctcatgattctgatttgtattaattttgagaattgccatggtagagaccctgctttgaggttatgttcttgagtcaggattcctggccagggattgtgatgatatatttctctttctgaagtggttcatgcaagaggttgtctgaaggaagagcaagaattgtagtgttattttgtggatacttgagacttataaaaaggctttttattttgtcacatttttgatacatgatgtttggcaaaaaacagacgatagtatttgcagagtgaatgaataagtggaacaggtgtgataatgagaggtcacacttgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtcgagatggcagtggccggtggggacagggctgagccagcaccaaccactcagcctttgagatcccgaggctggtctactgctgagaccttttgttagaagagaggagatcaagcatttgcaaggtttctgagtgtcaaaatatgaatccaagataactctttcacaatcctaacttcatgctgtctacaggtccatattttagcctgctttctccatgttcatccgaaaagaaagaaaagctaagggtggtggtcatatttgaaattagccagatcttaagtttttctgggggaaatttagaagaaaatatggaaaagtgactatgagcacatatacagctagtctttaaaacagttttatccaaaataaatgtatcacaaaattaataaaaatagttacttgcttgttttgaataattcaaatgatacaaaaattaataaaataaaaagtgcaaaaggccctcttatcaatgccaattctatttttttcagaaattaaacactgttaagattttagtgtgtatcctttcagaattcctgtgatttcatatatgtacaaatacaaacgtatctacataaagggaatcctactatacttgctattgtcattctattctctgctttttcatgtgagcatctttccatgtcactgatgcatacagaaattgcacatatgcatcagtgcatacagaaaattaaattttctgcatggttttccactgtatgtctggaccatagtttatttaataataatgccctttgggtaattatttatattgtttcctgctttttcaaagtaacagcttttgaaacaaatctctctctgtctttatataaatattgttgcattcctgtggaaatgtttctattggataacttcccaaaaggagatttattgcatcaaagataatatattcaaaaattttaaagatattgctaaattgtctagtaggtattttataccaatttatactcctcccaagaatgtatggagatatcttaatttctccatgccttcattaatgctgaaccatataagtagttttaatctttgctaattgaatagataaaaaatatctaatctaagtctagttcttaaaagttctatcttctaccaaaagtaatacacgtctattttagggagtaaaaatcacaagtaaggataaaaaatagtgcagcaataaacacaggagtgtagatgtctctgaacatactgatttaacttcctttggataaatacccagtagtaggactgctggatcatataataattctatctttagtttttttgaggacctccatactattcttcatagtggctgtactaatttacattcctaccaactgtgtatgaaggttcccttttctctacatccttgccagcattcattattgcttgtcatttggatacaatctattttaactggggtgagatgacatctcattgtagttttgatatgcatttctctgatgatcagtggtgttgagcaccttttcatatacctgtttgccatttgtatgtcttcctttgagaaatgtctattcagatattttacctattttaaaatcggattattagattgtttcctgtagagttgtttgagctccttgtatattctggttattaatctcttgtcagatgcatagcttacaaatattttctcccatcatgtggattgtgtcttcactttgtggattgtttactttgctgtgcagaagcttttaacttgatgcaatcccatttgtccacttttgctttggttgccttccacaggagtatttaaataaatgtagtttggtagattttggtatagtaatgcaggccagtgggagtcaggggagaaatgtgtagggaagtgagatagttctaaggatcctacaaacatgccttatgattgacttactcaatgtgaaagtcaatattaaacttgatgagctctagagatggtcatgcattttaaaaagaattactcaaaatattgtcttggaataccagagagcaagtgctttaagtataggctgggaagtaaaatgctaaaggaatgagaaggcatttggggttgagttcaacctaagaggcaggggagccacagggaaagacctagcacctgccacagaagagaattaggaagcagaattgaactataagcaattttgaggtgttcgttgggctgcagttgaaatattttttgaggttaatgagacatttgaaatggccgtgtattgtttaactcttgcatagtcctgcatagggaacaatctaataggatttctctgtgaatcaagtcttagaaatttgcttttaatttttatgaaaaacgcccatttctttgtttttgagacagagtcctgctctgtcatccaggctgggttgcagtggcgtgatcttggcccactgcaatctctgcctcctgggttcaggcaattttcctgtctcagcctcccgagtagctgggatttcaagtgcctgccaccatgcccggctaaatttttttgtatttttggtacagatggagtatcaccatgttggccaggctggtctcgaactcctgacctcaagtgattcaccagccttgacctcccaaagtgttgggatcacaggcatgagccactgtgcctgtgccccaaaacaccaatttctgatgtgtgatgcatgtaagatagaacaaacttcagtaaagcggggacttgaaaagaggctttggtaacagctgtcagcattaacccttgcccctccgtacctcctaatcccacccctgctcaaagtatgttcatctgagaatttgtctccataactatgtgactataaaaattctcatcgattttgttagttgatcaattgagggaaaaacatatgttacttgatataactggtgggtcaaaagaattaacccaggcaaatttgagataggtggatgggatgatggattgaaaatacagctgctctctttccaatcatgtactaagtaatttgggaaagattgatctaattgggtctagagagtacacttcacatggcattgtttgactttttttctgcatcgctagcgatctgtgcattacaactcaaatcagtcgggtttcctggcatatgtaattgccaatgttttttaccagaagagaaacattactcccacctcttcttattatgttacaaactatagtgctaatgaccatcgaccaacagtgactttcaggatgacctgtgtgagttttatctgaaaccatgtgaatttttcatcttaaaagtcccttagaatctcagtctatgtacactcaggtttgttgcaggtttagagttccgtgttttttgtttctaatgtagacacagccttataatttacaacagcattcactaattaaaattgtaagcataattactatccacgatacttattattagtttgcattcataaagctcaaaattcacttcatcctttcaagtagtgaataattagtttctttgggtttgcagctttatcatccttttatgacccatttggaagaaataaacaaccaaccccctggaagactgctttaaaaagctggaaatacattgtccagctagtacaatgaggctaatacaatgtggaaaatattacttttctttgattttagtagcctgtttatctttacatttactgaacaaataactattgagcacctaatgtatactgggacccttggggaggcaaagatgaatcaaagattctgtccttaaagaccttaaggtttttgtggaaggaaataaaactttacatgtatatatttaagcacttatatgtgtgtaacaggtataagtaaccataaacactgtcagaagaggaaataactctatgatcagcacctaacatgatatattaaggtagaagatttaatacatatcttttggaatacatgaataaataattgaatgtatttatttttattatttataagatacatcagtgggatattgatattggtcttaatatgacttgttttcattgttctcag

SEQ ID NO: 3 - DNA, 6472 bp, Homo sapiens
  source 1..6472: mol_type = genomic DNA
gtacctcagccagcatggcagcctctttcccacccaccttgggactcagttctgccccagatgaaattcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcacaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcagtttggaaaaatataatcaccacatctcaagtcacaaggaatcaaaagagatttttgtgcacattaataaagggggccggccccgccaacatcttctgtcgctgactcggagagctcagaagcaccggctgagggagctcaagctgcaagtcaaagcctttgctgacaaagaagaaggtggagatgtgaagtccgtgtgcatgaccttgttcctgctggctctgagggcgaggaatgagcacaggcaagctgatgagctggaggccatcatgcagggaaagggctctggcctgcagccagctgtttgcttggccatccgtgtcaacaccttcctcagctgcagtcagtaccacaagatgtacaggactgtgaaagccatcacagggagacagatttttcagcctttgcatgcccttcggaatgctgagaaggtacttctgccaggctaccaccactttgagtggcagccacctctgaagaatgtgtcttccagcactgatgttggcattattgatgggctgtctggactatcatcctctgtggatgattacccagtggacaccattgcaaagaggttccgctatgattcagctttggtgtctgctttgatggacatggaagaagacatcttggaaggcatgagatcccaagaccttgatgattacctgaatggccccttcactgtggtggtgaaggagtcttgtgatggaatgggagacgtgagtgagaagcatgggagtgggcctgtagttccagaaaaggcagtccgtttttcattcacaatcatgaaaattactattgcccacagctctcagaatgtgaaagtatttgaagaagccaaacctaactctgaactgtgttgcaagccattgtgccttatgctggcagatgagtctgaccacgagacgctgactgccatcctgagtcctctcattgctgagagggaggccatgaagagcagtgaattaatgcttgagctgggaggcattctccggactttcaagttcatcttcaggggcaccggctatgatgaaaaacttgtgcgggaagtggaaggcctcgaggcttctggctcagtctacatttgtactctttgtgatgccacccgtctggaagcctctcaaaatcttgtcttccactctataaccagaagccatgctgagaacctggaacgttatgaggtctggcgttccaacccttaccatgagtctgtggaagaactgcgggatcgggtgaaaggggtctcagctaaacctttcattgagacagtcccttccatagatgcactccactgtgacattggcaatgcagctgagttctacaagatcttccagctagagataggggaagtgtataagaatcccaatgcttccaaagaggaaaggaaaaggtggcaggccacactggacaagcatctccggaagaagatgaacctcaaaccaatcatgaggatgaatggcaactttgccaggaagctcatgaccaaagagactgtggatgcagtttgtgagttaattccttccgaggagaggcacgaggctctgagggagctgatggatctttacctgaagatgaaaccagtatggcgatcatcatgccctgctaaagagtgcccagaatccctctgccagtacagtttcaattcacagcgttttgctgagctcctttctacgaagttcaagtataggtatgagggaaaaatcaccaattattttcacaaaaccctggcccatgttcctgaaattattgagagggatggctccattggggcatgggcaagtgagggaaatgagtctggtaacaaactgtttaggcgcttccggaaaatgaatgccaggcagtccaaatgctatgagatggaagatgtcctgaaacaccactggttgtacacctccaaatacctccagaagtttatgaatgctcataatgcattaaaaacctctgggtttaccatgaaccctcaggcaagcttaggggacccattaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaagtgcatcggcgacattatctttaattgtatgtatttggtgcttcttcagggattgaactcagtatctttcattaaaaaacacagcagttttccttgctttttatatgcagaatatcaaagtcatttctaatttagttgtcaaaaacatatacatattttaacattagtttttttgaaaactcttggttttgtttttttggaaatgagtgggccactaagccacactttcccttcatcctgcttaatccttccagcatgtctctgcactaataaacagctaaattcacataatcatcctatttactgaagcatggtcatgctggtttatagattttttacccatttctactctttttctctattggtggcactgtaaatactttccagtattaaattatccttttctaacactgtaggaactattttgaatgcatgtgactaagagcatgatttatagcacaacctttccaataatcccttaatcagatcacattttgataaaccctgggaacatctggctgcaggaatttcaatatgtagaaacgctgcctatggttttttgcccttactgttgagactgcaatatcctagaccctagttttatactagagttttatttttagcaatgcctattgcaagtgcaattatatactccagggaaattcaccacactgaatcgagcatttgtgtgtgtatgtgtgaagtatatactgggacttcagaagtgcaatgtatttttctcctgtgaaacctgaatctacaagttttcctgccaagccactcaggtgcattgcagggaccagtgataatggctgatgaaaattgatgattggtcagtgaggtcaaaaggagccttgggattaataaacatgcactgagaagcaagaggaggagaaaaagatgtctttttcttccaggtgaactggaatttagttttgcctcagatttttttcccacaagatacagaagaagataaagatttttttggttgagagtgtgggtcttgcattacatcaaacagagttcaaattccacacagataagaggcaggatatataagcgccagtggtagttgggaggaataaaccattatttggatgcaggtggtttttgattgcaaatatgtgtgtgtcttcagtgattgtatgacagatgatgtattcttttgatgttaaaagattttaagtaagagtagatacattgtacccattttacattttcttattttaactacagtaatctacataaatatacctcagaaatcatttttggtgattattttttgttttgtagaattgcacttcagtttattttcttacaaataaccttacattttgtttaatggcttccaagagccttttttttttttgtatttcagagaaaattcaggtaccaggatgcaatggatttatttgattcaggggacctgtgtttccatgtcaaatgttttcaaataaaatgaaatatgagtttcaatactttttatattttaatatttccattcattaatattatggttattgtcagcaattttatgtttgaatatttgaaataaaagtttaagatttgaaaa

SEQ ID NO: 4 - protein, 1043 aa, Homo sapiens
MAASFPPTLGLSSAPDEIQHPHIKFSEWKFKLFRVRSFEKTPEEAQKEKKDSFEGKPSLEQSPAVLDKADGQKPVPTQPLLKAHPKFSKKFHDNEKARGKAIHQANLRHLCRICGNSFRADEHNRRYPVHGPVDGKTLGLLRKKEKRATSWPDLIAKVFRIDVKADVDSIHPTEFCHNCWSIMHRKFSSAPCEVYFPRNVTMEWHPHTPSCDICNTARRGLKRKSLQPNLQLSKKLKTVLDQARQARQHKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 5 - protein, 931 aa, Homo sapiens
MAASFPPTLGLSSAPDEIQHPHIKFSEWKFKLFRVRSFEKTPEEAQKEKKDSFEGKPSLEQSPAVLDKADGQKPVPTQPLLKAHPKFSKKFHDNEKARGKAIHQANLRHLCRICGNSFRADEHNRRYPVHGPVDGKTLGLLRKKEKRATSWPDLIAKVFRIDVKADVDSIHPTEFCHNCWSIMHRKFSSAPCEVYFPRNVTMEWHPHTPSCDICNTARRGLKRKSLQPNLQLSKKLKTVLDQARQARQHKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRN

SEQ ID NO: 6 - protein, 1043 aa, Homo sapiens
MAASFPPTLGLSSAPDEIQHPHIKFSEWKFKLFRVRSFEKTPEEAQKEKKDSFEGKPSLEQSPAVLDKADGQKPVPTQPLLKAHPKFSKKFHDNEKARGKAIHQANLRHLCRICGNSFRADEHNRRYPVHGPVDGKTLGLLRKKEKRATSWPDLIAKVFRIDVKADVDSIHPTEFCHNCWSIMHRKFSSAPCEVYFPRNVTMEWHPHTPSCDICNTARRGLKRKSLQPNLQLSKKLKTVLDQARQARQRKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 7 - protein, 774 aa, synthetic construct
SKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 8 - protein, 759 aa, synthetic construct
SKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDP

SEQ ID NO: 9 - protein, 841 aa, synthetic construct
EWHPHTPSCDICNTARRGLKRKSLQPNLQLSKKLKTVLDQARQARQRKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 10 - protein, 826 aa, synthetic construct
EWHPHTPSCDICNTARRGLKRKSLQPNLQLSKKLKTVLDQARQARQRKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDP

SEQ ID NO: 11 - protein, 836 aa, synthetic construct
EVYFPRNVTMEWHPHTPSCDICNTARRGLKRKSLQPNLQLSKKLKTVLDQARQARQRKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDP

SEQ ID NO: 12 - protein, 811 aa, synthetic construct
RRGLKRKSLQPNLQLSKKLKTVLDQARQARQRKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDP

SEQ ID NO: 13 - protein, 832 aa, synthetic construct
PRNVTMEWHPHTPSCDICNTARRGLKRKSLQPNLQLSKKLKTVLDQARQARQRKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDP

SEQ ID NO: 14 - protein, 660 aa, synthetic construct
LEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDP

SEQ ID NO: 15 - DNA, 3131 bp, synthetic construct
  source 1..3131: mol_type = other DNA
atggccgcctccttcccacctacccttggattgtcctccgcccctgacgaaattcaacatccccacatcaaattctcggagtggaagttcaagctctttcgcgtgcgctcgttcgaaaagacccccgaggaagcccaaaaggagaagaaagactcattcgaaggaaaacccagcctcgaacagtccccggccgtcctggacaaggccgacgggcagaagcctgtgccgacccagccgctgctgaaagcgcacccgaaattctccaagaagtttcacgataacgagaaggcccggggaaaggccatccaccaagcaaaccttagacacctgtgccgcatctgtgggaactcattcagagccgacgaacataaccggagataccctgtgcatggccctgtcgacggaaagaccctggggctcctgagaaagaaggagaagagggcgacatcctggccggacctgatcgcaaaggtgttcagaatcgacgtgaaggcagatgtggacagcatccacccaaccgagttctgccacaactgctggagcattatgcaccggaagttcagctcagcgccctgtgaagtgtacttcccgcgcaacgtgactatggagtggcatccacacactccgtcctgcgacatctgtaacactgctcggcgcggactcaagaggaagtccctgcagccgaatctgcagctgagcaagaagcttaagaccgtgctggaccaggctcggcaggcccgccagcacaagcgacgcgcccaggcccggatctcatctaaggatgtgatgaagaagatcgccaattgcagcaaaatccacctgtctaccaagctgctggcggtggacttcccggagcacttcgtgaagtccatcagctgtcagatctgcgagcatattctcgccgaccccgtggagactaattgcaagcacgtgttctgccgcgtgtgcatcctgcgctgcctgaaggtcatgggctcctattgcccttcctgccggtacccctgtttccctactgatctggagtccccggtcaagtccttcttgtccgtgctgaactccctgatggtcaaatgtcccgcaaaggagtgcaatgaggaagtgtccctggaaaagtacaaccaccacatcagcagccacaaggagtccaaagaaatctttgtgcacattaacaagggcggtcggccccggcagcatctgctctcgctgactcgccgggcccagaagcacaggctccgggagctgaagctgcaagtcaaggccttcgccgacaaggaagagggaggagatgtgaagtccgtgtgcatgaccctgtttttgctggcgctgcgggctcggaacgaacacagacaagctgatgaactggaggccatcatgcagggcaaaggatcgggactccagccggctgtgtgtctcgccatccgcgtcaacacattcctctcatgctcccaataccacaagatgtacaggactgtgaaggccatcaccggacggcagatctttcagccactccacgcccttcggaacgcagaaaaggtcttgctgccgggataccatcatttcgaatggcagccgcccttgaaaaacgtgtcctcgtccaccgacgtgggcattattgatgggctgagcggcctgtcctcctctgtggatgactaccctgtggataccatcgccaaacggttcagatacgattccgcgctggtgtcggccctgatggacatggaggaggacatcctggagggaatgagatcacaagatctggacgactacctcaacgggcccttcacggtggtggtcaaggaatcgtgcgatggaatgggcgacgtgtcggagaagcacggttccggacctgtggtgccggaaaaggccgtgcgcttctccttcaccatcatgaagatcaccattgcgcatagctcccagaacgtcaaagtgttcgaagaggccaagccgaactcagagctctgctgcaagccgctgtgcctgatgttggcggacgagagcgatcacgaaaccctgaccgccattctgtcgcctctgatcgcggagagggaggccatgaagtcctccgaactgatgctggagctgggcggtattttgcggacttttaagttcatcttccggggaaccggttatgacgaaaagctcgtgcgcgaagtggagggcctggaagcctcaggctccgtctacatctgcactctctgcgacgccacccggctggaggcgtcacagaatcttgtgttccactcgatcactaggtcccacgcggagaacctggaacgctatgaggtctggcgctctaacccataccacgaatccgtggaagaacttcgggacagagtgaagggagtgtcagcaaagcctttcattgaaaccgtgcctagcatcgacgccctccattgcgacatcggcaacgccgccgagttctacaagatcttccagcttgagatcggggaagtgtacaagaacccgaacgcctccaaggaagaaagaaagcggtggcaggctacccttgacaaacacctccgcaagaagatgaacctgaagcccattatgcggatgaacggaaacttcgctaggaagctgatgactaaggaaacggtcgacgcggtctgtgaactgatccccagcgaagaacgacatgaagcgctgcgcgaactcatggacctgtacctgaagatgaagcctgtctggcggagctcgtgccctgccaaggagtgcccggagtcgctgtgtcagtacagctttaacagccaaaggttcgcagagctgctgtcgaccaagttcaagtacagatacgaaggaaagattaccaactacttccacaagactctcgctcacgtgcccgagattatcgaacgcgatggttccatcggggcctgggcctccgagggcaacgagtcgggcaacaagttgttccgccggtttagaaagatgaacgcccgccagtccaagtgctacgaaatggaagatgtgctgaagcatcactggctgtatacctccaagtacctccagaagttcatgaacgcacataacgccctcaagacctccgggttcaccatgaacccccaggcctccctcggtgaccctctgggaattgaagatagcttggagagccaggactcgatggaattcta

SEQ ID NO: 16 - DNA, 3132 bp, synthetic construct
  source 1..3132: mol_type = other DNA
atggccgccagctttcctcctacactgggactgtctagcgcccctgacgagattcagcaccctcacatcaagttcagcgagtggaagttcaagctgttcagagtgcggagcttcgagaaaacccctgaggaagcccagaaagagaagaaggacagcttcgagggcaagcccagcctggaacagtctcctgctgtgctggataaggccgacggccagaaacctgtgcctacacagcctctgctgaaggctcaccccaagttctccaagaagttccacgacaacgagaaggccagaggcaaggccatccaccaggccaatctgagacacctgtgccggatctgcggcaacagcttcagagccgacgagcacaatcggagataccctgtgcacggccctgtggatggaaagactctgggcctgctgcggaagaaagaaaagagagccaccagctggcccgacctgatcgccaaggtgttcagaatcgacgtgaaggccgatgtggacagcattcaccccaccgagttctgccacaactgctggtccatcatgcaccggaagttcagctctgccccttgcgaggtgtacttccccagaaacgtgaccatggaatggcacccacacacacccagctgcgacatctgcaacacagccagaagaggcctgaagcggaagtccctgcagcctaatctgcagctgagcaagaaactgaaaaccgtgctggaccaggccagacaggcccggcaaagaaaaagacgcgcccaggctagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgccccagctgtagatacccttgcttccccaccgacctggaaagccctgtgaagtcctttctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagaagggcccagaagcaccggctgcgggaactgaagctgcaagtgaaggcctttgccgacaaagaggaaggcggcgacgtcaagagcgtgtgcatgaccctgtttctgctggccctgagagcccggaatgagcatagacaggccgatgagctggaagccattatgcaaggcaaaggcagcggactgcagcctgctgtgtgtctggctatcagagtgaataccttcctgagctgcagccagtaccacaagatgtaccggaccgtgaaagccatcaccggcagacagatcttccagccactgcacgccctgagaaacgccgagaaagttctgctgcctggctaccaccacttcgagtggcagcctccactgaagaacgtgtccagcagcaccgacgtgggcatcatcgatggactgtctggactgagcagcagcgtggacgattaccccgtggacacaatcgccaagagattcagatacgacagcgccctggtgtctgccctgatggacatggaagaggacatcctggaaggcatgcggagccaggacctggacgactatctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaggtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctcgagagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgcggatgaacggcaacttcgcccggaagctgatgaccaaagaaaccgtggatgccgtgtgcgagctgatcccctctgaggaaagacacgaggccctgagggaactgatggacctgtacctgaagatgaagcccgtgtggcggtctagctgtcctgccaaagagtgccctgagtctctgtgccagtacagcttcaacagccagagattcgccgagctgctgagcacaaagttcaagtacagatacgaggggaagatcacgaactacttccacaagaccctggctcacgtgcccgagatcatcgagagagatggctctattggcgcctgggcctctgagggcaatgagtctggcaacaagctgtttcggcggttcagaaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggagatcctctgggcattgaggatagcctggaatcccaggacagcatggaattctga

SEQ ID NO: 17 - DNA, 2327 bp, synthetic construct
  source 1..2327: mol_type = other DNA
gcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtgtacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgaccctctgggaattgaggatagcctggaatcccaggacagcatggaattctga

SEQ ID NO: 18 - DNA, 2281 bp, synthetic construct
  source 1..2281: mol_type = other DNA
gcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtgtacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatccttt

SEQ ID NO: 19 - DNA, 2529 bp, synthetic construct
  source 1..2529: mol_type = other DNA
gaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtgtacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgaccctctgggaattgaggatagcctggaatcccaggacagcatggaattctgataa

SEQ ID NO: 20 - DNA, 2480 bp, synthetic construct
  source 1..2480: mol_type = other DNA
gaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtgtacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatccttt

SEQ ID NO: 21 - DNA, 2512 bp, synthetic construct
  source 1..2512: mol_type = other DNA
gcgaagtgtacttccccagaaacgtgaccatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatccttt

SEQ ID NO: 22 - DNA, 2436 bp, synthetic construct
  source 1..2436: mol_type = other DNA
tagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatccttt

SEQ ID NO: 23 - DNA, 2498 bp, synthetic construct
  source 1..2498: mol_type = other DNA
cccagaaacgtgaccatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatccttt

SEQ ID NO: 24 - DNA, 1984 bp, synthetic construct
  source 1..1984: mol_type = other DNA
ccctagaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatccttt

SEQ ID NO: 25 - DNA, 50 bp, synthetic construct
  source 1..50: mol_type = other DNA
gacctggagagtccagtgaagtcctttctgagcgtcttgaattccctgat

SEQ ID NO: 26 - DNA, 50 bp, synthetic construct
  source 1..50: mol_type = other DNA
tgacctggagagtccagtgaagtcctttctgagcgtcttgaattccctga

SEQ ID NO: 27 - DNA, 50 bp, synthetic construct
  source 1..50: mol_type = other DNA
tctgagcgtcttgaattccctgatggtgaaatgtccagcaaaagagtgca

SEQ ID NO: 28 - DNA, 50 bp, synthetic construct
  source 1..50: mol_type = other DNA
gggtctgcattctcagatgcctcaaagtcatgggcagctattgtccctct

SEQ ID NO: 29 - DNA, 50 bp, synthetic construct
  source 1..50: mol_type = other DNA
agctcaggcaaggatcagcagcaaggatgtcatgaagaagatcgccaact

SEQ ID NO: 30 - DNA, 50 bp, synthetic construct
  source 1..50: mol_type = other DNA
agtttagcagtgccccatgtgaggtttacttcccgaggaacgtgaccatg

SEQ ID NO: 31 - DNA, 50 bp, synthetic construct
  source 1..50: mol_type = other DNA
ctgccataactgctggagcatcatgcacaggaagtttagcagtgccccat

SEQ ID NO: 32 - DNA, 50 bp, synthetic construct
  source 1..50: mol_type = other DNA
ggagtggcacccccacacaccatcctgtgacatctgcaacactgcccgtc

SEQ ID NO: 33 - DNA, 50 bp, synthetic construct
  source 1..50: mol_type = other DNA
atggagtggcacccccacacaccatcctgtgacatctgcaacactgcccg

SEQ ID NO: 34 - DNA, 50 bp, synthetic construct
  source 1..50: mol_type = other DNA
tgccataactgctggagcatcatgcacaggaagtttagcagtgccccatg

SEQ ID NO: 35 - DNA, 50 bp, synthetic construct
  source 1..50: mol_type = other DNA
ggagcatcatgcacaggaagtttagcagtgccccatgtgaggtttacttc

SEQ ID NO: 36 - DNA, 50 bp, synthetic construct
  source 1..50: mol_type = other DNA
ccatggagtggcacccccacacaccatcctgtgacatctgcaacactgcc

SEQ ID NO: 37 - DNA, 50 bp, synthetic construct; identical to SEQ ID NO: 31
  source 1..50: mol_type = other DNA

SEQ ID NO: 38 - DNA, 50 bp, synthetic construct
  source 1..50: mol_type = other DNA
gaattccctgatggtgaaatgtccagcaaaagagtgcaatgaggaggtca

SEQ ID NO: 39 - DNA, 50 bp, synthetic construct
  source 1..50: mol_type = other DNA
agctcaggcaaggatcagcagcaaggatgtcatgaagaagatcgcaaact

SEQ ID NO: 40 - DNA, 50 bp, synthetic construct
  source 1..50: mol_type = other DNA
agtttagcagtgccccatgtgaggtttacttcccgaggaatgtcactatg

SEQ ID NO: 41 - DNA, 50 bp, synthetic construct
  source 1..50: mol_type = other DNA
ctgccataactgctggagcatcatgcacaggaagtttagcagtgcaccat

SEQ ID NO: 42 - DNA, 50 bp, synthetic construct
  source 1..50: mol_type = other DNA
accatggagtggcacccccacacaccatcctgtgacatctgcaacactgc

SEQ ID NO: 43 - DNA, 50 bp, synthetic construct; identical to SEQ ID NO: 35
  source 1..50: mol_type = other DNA

SEQ ID NO: 44 - DNA, 50 bp, synthetic construct; identical to SEQ ID NO: 38
  source 1..50: mol_type = other DNA

SEQ ID NO: 45 - DNA, 50 bp, synthetic construct
  source 1..50: mol_type = other DNA
ggtgaaatgtccagcaaaagagtgcaatgaggaggtcagtttggaaaaat

SEQ ID NO: 46 - DNA, 50 bp, synthetic construct
  source 1..50: mol_type = other DNA
tggtgaaatgtccagcaaaagagtgcaatgaggaggtcagtttggaaaaa

SEQ ID NO: 47 - DNA, 50 bp, synthetic construct
  source 1..50: mol_type = other DNA
atgaggaggtcagtttggaaaaatataatcaccacatctcaagtcacaag

SEQ ID NO: 48 - DNA, 50 bp, synthetic construct
  source 1..50: mol_type = other DNA
tgccgatatccatgcttccctactgacctggagagtccagtgaagtcctt

SEQ ID NO: 49 - DNA, 50 bp, synthetic construct
  source 1..50: mol_type = other DNA
gcagtaagatacatcttagtaccaagctccttgcagtggacttcccagag

SEQ ID NO: 50 - DNA, 50 bp, synthetic construct
  source 1..50: mol_type = other DNA
gagtggcacccccacacaccatcctgtgacatctgcaacactgcccgtcg

SEQ ID NO: 51 - DNA, 50 bp, synthetic construct
  source 1..50: mol_type = other DNA
gtgaggtttacttcccgaggaacgtgaccatggagtggcacccccacaca

SEQ ID NO: 52 - DNA, 50 bp, synthetic construct
  source 1..50: mol_type = other DNA
ggggactcaagaggaagagtcttcagccaaacttgcagctcagcaaaaaa

SEQ ID NO: 53 - DNA, 50 bp, synthetic construct
  source 1..50: mol_type = other DNA
tcggggactcaagaggaagagtcttcagccaaacttgcagctcagcaaaa

SEQ ID NO: 54 - DNA, 50 bp, synthetic construct
  source 1..50: mol_type = other DNA
tgaggtttacttcccgaggaacgtgaccatggagtggcacccccacacac

SEQ ID NO: 55 - DNA, 50 bp, synthetic construct
  source 1..50: mol_type = other DNA
ccgaggaacgtgaccatggagtggcacccccacacaccatcctgtgacat

SEQ ID NO: 56 - DNA, 50 bp, synthetic construct
  source 1..50: mol_type = other DNA
cgtcggggactcaagaggaagagtcttcagccaaacttgcagctcagcaa

SEQ ID NO: 57 - DNA, 50 bp, synthetic construct; identical to SEQ ID NO: 51
  source 1..50: mol_type = other DNA

SEQ ID NO: 58 - DNA, 50 bp, synthetic construct
  source 1..50: mol_type = other DNA
gtttggaaaaatataatcaccacatctcaagtcacaaggaatcaaaagag

SEQ ID NO: 59 - DNA, 100 bp, synthetic construct
  source 1..100: mol_type = other DNA
gcagtaagatacatcttagtaccaagctccttgcagtggacttcccagagcactttgtgaaatccatctcctgccagatctgtgaacacattctggctga

SEQ ID NO: 60 - DNA, 100 bp, synthetic construct
  source 1..100: mol_type = other DNA
gagtggcacccccacacaccatcctgtgacatctgcaacactgcccgtcggggactcaagaggaagagtcttcagccaaacttgcagctcagcaaaaaac

SEQ ID NO: 61 - DNA, 100 bp, synthetic construct
  source 1..100: mol_type = other DNA
caaagcctttgctgacaaagaagaaggtggagatgtgaagtccgtgtgcatgaccttgttcctgctggctctgagggcgaggaatgagcacaggcaagct

SEQ ID NO: 62 - DNA, 100 bp, synthetic construct
  source 1..100: mol_type = other DNA
cagccacctctgaagaatgtgtcttccagcactgatgttggcattattgatgggctgtctggactatcatcctctgtggatgattacccagtggacacca

SEQ ID NO: 63 - DNA, 100 bp, synthetic construct
  source 1..100: mol_type = other DNA
tcacaatcatgaaaattactattgcccacagctctcagaatgtgaaagtatttgaagaagccaaacctaactctgaactgtgttgcaagccattgtgcct

SEQ ID NO: 64 - DNA, 100 bp, synthetic construct
  source 1..100: mol_type = other DNA
tctttgtgatgccacccgtctggaagcctctcaaaatcttgtcttccactctataaccagaagccatgctgagaacctggaacgttatgaggtctggcgt

SEQ ID NO: 65 - DNA, 100 bp, synthetic construct
  source 1..100: mol_type = other DNA
tggacaagcatctccggaagaagatgaacctcaaaccaatcatgaggatgaatggcaactttgccaggaagctcatgaccaaagagactgtggatgcagt

SEQ ID NO: 66 - DNA, 100 bp, synthetic construct
  source 1..100: mol_type = other DNA
caaaaccctggcccatgttcctgaaattattgagagggatggctccattggggcatgggcaagtgagggaaatgagtctggtaacaaactgtttaggcgc

SEQ ID NO: 67 - DNA, 50 bp, synthetic construct
  source 1..50: mol_type = other DNA
aggcatagaggactctctggaaagccaagattcaatggaattttaagtag

SEQ ID NO: 68 - DNA, 100 bp, synthetic construct
  source 1..100: mol_type = other DNA
gtagggcaaccacttatgagttggtttttgcaattgagtttccctctgggttgcattgagggcttctcctagcaccctttactgctgtgtatggggcttc

SEQ ID NO: 69 - DNA, 503 bp, synthetic construct
  source 1..503: mol_type = other DNA
gatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgcaaact

SEQ ID NO: 70 - DNA, 400 bp, synthetic construct
  source 1..400: mol_type = other DNA
aaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgcaaact

SEQ ID NO: 71 - DNA, 503 bp, synthetic construct
  source 1..503: mol_type = other DNA
tgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaatgtcactatg

SEQ ID NO: 72 - DNA, 243 bp, synthetic construct
  source 1..243: mol_type = other DNA
gagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaatgtcactatg

SEQ ID NO: 73 - DNA, 230 bp, synthetic construct
  source 1..230: mol_type = other DNA
gaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgcaccat

SEQ ID NO: 74 - DNA, 269 bp, synthetic construct
  source 1..269: mol_type = other DNA
ccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgc

SEQ ID NO: 75 - DNA, 230 bp, synthetic construct
  source 1..230: mol_type = other DNA
ctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttc

SEQ ID NO: 76 - DNA, 500 bp, synthetic construct
  source 1..500: mol_type = other DNA
catggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtca

SEQ ID NO: 77 - DNA, 250 bp, synthetic construct
  source 1..250: mol_type = other DNA
gcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaa

SEQ ID NO: 78 - DNA, 250 bp, synthetic construct
  source 1..250: mol_type = other DNA
gagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagc

SEQ ID NO: 79 - DNA, 1800 bp, synthetic construct
  source 1..1800: mol_type = other DNA
aggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaag

SEQ ID NO: 80 - DNA, 900 bp, synthetic construct
  source 1..900: mol_type = other DNA
aggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagct

SEQ ID NO: 81 - DNA, 50 bp, synthetic construct
  source 1..50: mol_type = other DNA
tgctgtgtggagggaggcacgcctgtagctctgatgtcagatggcaatgt

SEQ ID NO: 82 - DNA, 50 bp, synthetic construct
  source 1..50: mol_type = other DNA
aagagagctacttcctggccggacctcattgccaaggttttccggatcga

SEQ ID NO: 83 - DNA, 50 bp, synthetic construct
  source 1..50: mol_type = other DNA
aagcaagaggcaaagcgatccatcaagccaaccttcgacatctctgccgc

SEQ ID NO: 84 - DNA, 50 bp, synthetic construct
  source 1..50: mol_type = other DNA
cagcatggcagcctctttcccacccaccttgggactcagttctgccccag

SEQ ID NO: 85 - DNA, 50 bp, synthetic construct
  source 1..50: mol_type = other DNA
ttgtgtacagactaagttgaagatgttaggagggaagattgtgggccaag

SEQ ID NO: 86 - DNA, 50 bp, synthetic construct
  source 1..50: mol_type = other DNA
ttactcccacctcttcttattatgttacaaactatagtgctaatgaccat

SEQ ID NO: 87 - DNA, 50 bp, synthetic construct
  source 1..50: mol_type = other DNA
acagaagagaattaggaagcagaattgaactataagcaattttgaggtgt

SEQ ID NO: 88 - DNA, 50 bp, synthetic construct
  source 1..50: mol_type = other DNA
gggaagtaaaatgctaaaggaatgagaaggcatttggggttgagttcaac

SEQ ID NO: 89 - DNA, 50 bp, synthetic construct
  source 1..50: mol_type = other DNA
aaccaaccccctggaagactgctttaaaaagctggaaatacattgtccag

SEQ ID NO: 90 - DNA, 50 bp, synthetic construct
  source 1..50: mol_type = other DNA
cacagaagagaattaggaagcagaattgaactataagcaattttgaggtg

SEQ ID NO: 91 - DNA, 50 bp, synthetic construct
  source 1..50: mol_type = other DNA
ggcagtggccggtggggacagggctgagccagcaccaaccactcagcctt

SEQ ID NO: 92 - DNA, 50 bp, synthetic construct
  source 1..50: mol_type = other DNA
ttttttctgcatcgctagcgatctgtgcattacaactcaaatcagtcggg

SEQ ID NO: 93 - DNA, 290 bp, synthetic construct
  source 1..290: mol_type = other DNA
tgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgt

SEQ ID NO: 94 - DNA, 1792 bp, synthetic construct
  source 1..1792: mol_type = other DNA
aggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagtt

SEQ ID NO: 95 - DNA, 25 bp, synthetic construct
  source 1..25: mol_type = other DNA
ctgacctcttctcttcctcccacag

SEQ ID NO: 96 - skipped

SEQ ID NO: 97 - DNA, 226 bp, synthetic construct
  source 1..226: mol_type = other DNA
gctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 98 - DNA, 226 bp, synthetic construct
  source 1..226: mol_type = other DNA
actgtgccttctagttgccagccatctgttgtttgcccctcccccgtgccttccttgaccctggaaggtgccactcccactgccctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 99 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 100 - DNA, 12 bp, synthetic construct
  source 1..12: mol_type = other DNA
gccgccaccatg

SEQ ID NO: 101 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 102 - DNA, 3381 bp, synthetic construct
  source 1..3381: mol_type = other DNA
gtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaagtgcatcggcgacattatctttaattgtatgtatttggtgcttcttcagggattgaactcagtatctttcattaaaaaacacagcagttttccttgctttttatatgcagaatatcaaagtcatttctaatttagttgtcaaaaacatatacatattttaacattagtttttttgaaaactcttggttttgtttttttggaaatgagtgggccactaagccacactttcccttcatcctgcttaatccttccagcatgtctctgcactaataaacagctaaattcacataatcatcctatttactgaagcatggtcatgctggtttatagattttttacccatttctactctttttctctattggtggcactgtaaatactttccagtattaaattatccttttctaacactgtaggaactattttgaatgcatgtgactaagagcatgatttatagcacaacctttccaataatcccttaatcagatcacattttgataaaccctgggaacatctggctgcaggaatttcaatatgtagaaacgctgcctatggttttttgcccttactgttgagactgcaatatcctagaccctagttttatactagagttttatttttagcaatgcctattgcaagtgcaattatatactccagggaaattcaccacactgaatcgagcatttgtgtgtgtatgtgtgaagtatatactgggacttcagaagtgcaatgtatttttctcctgtgaaacctgaatctacaagttttcctgccaagccactcaggtgcattgcagggaccagtgataatggctgatgaaaattgatgattggtcagtgaggtcaaaaggagccttgggattaataaacatgcactgagaagcaagaggaggagaaaaagatgtctttttcttccaggtgaactggaatttagttttgcctcagatttttttcccacaagatacagaagaagataaagatttttttggttgagagtgtgggtcttgcattacatcaaacagagttcaaattccacacagataagaggcaggatatataagcgccagtggtagttgggaggaataaaccattatttggatgcaggtggtttttgattgcaaatatgtgtgtgtcttcagtgattgtatgacagatgatgtattcttttgatgttaaaagattttaagtaagagtagatacattgtacccattttacattttcttattttaactacagtaatctacataaatatacctcagaaatcatttttggtgattattttttgttttgtagaattgcacttcagtttattttcttacaaataaccttacattttgtttaatggcttccaagagccttttttttttttgtatttcagagaaaattcaggtaccaggatgcaatggatttatttgattcaggggacctgtgtttccatgtcaaatgttttcaaataaaatgaaatatgagtttcaatactttttatattttaatatttccattcattaatattatggttattgtcagcaattttatgtttgaatatttgaaataaaagtttaagatttgaaaatggtatgtattataatttctattcaaatattaataataatattgagtgcagcatt

SEQ ID NO: 103 - DNA, 607 bp, synthetic construct
  source 1..607: mol_type = other DNA
gaattaactcgaggaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccaacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 104 - DNA, 843 bp, synthetic construct
  source 1..843: mol_type = other DNA
atgggagctggtgctaccggcagagctatggatggacctagactgctgctcctgctgctgctcggagtttctcttggcggagccaaagaggcctgtcctaccggcctgtatacacactctggcgagtgctgcaaggcctgcaatcttggagaaggcgtggcacagccttgcggcgctaatcagacagtgtgcgagccttgcctggacagcgtgacctttagcgacgtggtgtctgccaccgagccatgcaagccttgtaccgagtgtgtgggcctgcagagcatgtctgccccttgtgtggaagccgacgatgccgtgtgtagatgcgcctacggctactaccaggacgagacaacaggcagatgcgaggcctgtagagtgtgtgaagccggctctggactggtgttcagctgccaagacaagcagaacaccgtgtgcgaggaatgccccgatggcacctatagcgacgaggccaaccatgtagatccctgcctgccttgtactgtgtgcgaagataccgagcggcagctgcgcgagtgtacaagatgggctgatgccgagtgcgaagagatccccggcagatggatcaccagaagcacacctccagagggcagcgatagcacagccccttctacacaagagcccgaggctcctcctgagcaggatctgattgcctctacagtggccggcgtggtcacaacagtgatgggatcttctcagcccgtggtcaccagaggcaccaccgacaatctgatccccgtgtactgtagcatcctggccgccgtggttgtgggactcgtggcctatatcgccttcaagcggtggaaccggggcatcctgtaa

SEQ ID NO: 105 - DNA, 78 bp, synthetic construct
  source 1..78: mol_type = other DNA
atgaggaccgaggcccccgagggcaccgagagcgagatggagacccccagcgccatcaacggcaaccccagctggcac

SEQ ID NO: 106 - DNA, 3083 bp, synthetic construct
  source 1..3083: mol_type = other DNA
gatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgcaaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtgtacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgaccctctgggaattgaggatagcctggaatcccaggacagcatggaattctgataagcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaa

SEQ ID NO: 107 - DNA, 4481 bp, synthetic construct
  source 1..4481: mol_type = other DNA
aaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgcaaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtgtacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctttaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaag

SEQ ID NO: 108 - DNA, 3581 bp, synthetic construct
  source 1..3581: mol_type = other DNA
aaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgcaaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtgtacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctttaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagct

SEQ ID NO: 109 - DNA, 3282 bp, synthetic construct
  source 1..3282: mol_type = other DNA
tgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaatgtcactatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtgtacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgaccctctgggaattgaggatagcctggaatcccaggacagcatggaattctgataagagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagc

SEQ ID NO: 110 - DNA, 4523 bp, synthetic construct
  source 1..4523: mol_type = other DNA
gagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaatgtcactatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtgtacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctttaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaag

SEQ ID NO: 111 - DNA, 3623 bp, synthetic construct
  source 1..3623: mol_type = other DNA
gagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaatgtcactatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtgtacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctttaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagct

SEQ ID NO: 112 - DNA, 4542 bp, synthetic construct
  source 1..4542: mol_type = other DNA
gaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgcaccatgcgaagtgtacttccccagaaacgtgaccatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctttaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaag

SEQ ID NO: 113 - DNA, 4505 bp, synthetic construct
  source 1..4505: mol_type = other DNA
ccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttcccgaggaacgtgaccatggagtggcacccccacacaccatcctgtgacatctgcaacactgctagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctttaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaag

SEQ ID NO: 114 - DNA, 4528 bp, synthetic construct
  source 1..4528: mol_type = other DNA
ctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttccccagaaacgtgaccatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctttaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaag

SEQ ID NO: 115 - DNA, 4284 bp, synthetic construct
  source 1..4284: mol_type = other DNA
catggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtgcaatgaggaggtcaccctagaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctttaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagttgtcaaaag

SEQ ID NO: 116 - DNA, 5254 bp, synthetic construct
  source 1..5254: mol_type = other DNA
tgagcacacagttattacttggaaattgtgtacagactaagttgaagatgttaggagggaagattgtgggccaagtaacggggtgtatgtgtgtgggtatagggtgggcagctgggatggaaatggggggctgctgctgctgctgcaccctggcctcctgaactaatgatatcactcaccagaaactactgttcctgcactgtccaagccaccccaaactagtttgtcaaaatgaatctgtgctgtgtggagggaggcacgcctgtagctctgatgtcagatggcaatgtgaattcctgacctcttctcttcctcccacaggccgccaccatggccgccagctttcctcctacactgggactgtctagcgcccctgacgagattcagcaccctcacatcaagttcagcgagtggaagttcaagctgttcagagtgcggagcttcgagaaaacccctgaggaagcccagaaagagaagaaggacagcttcgagggcaagcccagcctggaacagtctcctgctgtgctggataaggccgacggccagaaacctgtgcctacacagcctctgctgaaggctcaccccaagttctccaagaagttccacgacaacgagaaggccagaggcaaggccatccaccaggccaatctgagacacctgtgccggatctgcggcaacagcttcagagccgacgagcacaatcggagataccctgtgcacggccctgtggatggaaagactctgggcctgctgcggaagaaagaaaagagagccaccagctggcccgacctgatcgccaaggtgttcagaatcgacgtgaaggccgatgtggacagcattcaccccaccgagttctgccacaactgctggtccatcatgcaccggaagttcagctctgccccttgcgaggtgtacttccccagaaacgtgaccatggaatggcacccacacacacccagctgcgacatctgcaacacagccagaagaggcctgaagcggaagtccctgcagcctaatctgcagctgagcaagaaactgaaaaccgtgctggaccaggccagacaggcccggcaaagaaaaagacgcgcccaggctagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgccccagctgtagatacccttgcttccccaccgacctggaaagccctgtgaagtcctttctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagaagggcccagaagcaccggctgcgggaactgaagctgcaagtgaaggcctttgccgacaaagaggaaggcggcgacgtcaagagcgtgtgcatgaccctgtttctgctggccctgagagcccggaatgagcatagacaggccgatgagctggaagccattatgcaaggcaaaggcagcggactgcagcctgctgtgtgtctggctatcagagtgaataccttcctgagctgcagccagtaccacaagatgtaccggaccgtgaaagccatcaccggcagacagatcttccagccactgcacgccctgagaaacgccgagaaagttctgctgcctggctaccaccacttcgagtggcagcctccactgaagaacgtgtccagcagcaccgacgtgggcatcatcgatggactgtctggactgagcagcagcgtggacgattaccccgtggacacaatcgccaagagattcagatacgacagcgccctggtgtctgccctgatggacatggaagaggacatcctggaaggcatgcggagccaggacctggacgactatctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaggtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctcgagagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgcggatgaacggcaacttcgcccggaagctgatgaccaaagaaaccgtggatgccgtgtgcgagctgatcccctctgaggaaagacacgaggccctgagggaactgatggacctgtacctgaagatgaagcccgtgtggcggtctagctgtcctgccaaagagtgccctgagtctctgtgccagtacagcttcaacagccagagattcgccgagctgctgagcacaaagttcaagtacagatacgaggggaagatcacgaactacttccacaagaccctggctcacgtgcccgagatcatcgagagagatggctctattggcgcctgggcctctgagggcaatgagtctggcaacaagctgtttcggcggttcagaaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggagatcctctgggcattgaggatagcctggaatcccaggacagcatggaattctgaaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagttaacatttacacagtgctttttaccactgtggaatgttttcacactcatttttccttacaacaattctgaggagtaggtgttgttattatctccatttgatgggggtttaaatgatttgctcaaagtcatttaggggtaataaatacttggcttggaaatttaacacagtccttttgtctccaaagcccttcttctttccaccacaaattaatcactatgtttataaggtagtatcagaatttttttaggattcacaactaatcactatagcacatgaccttgggattacatttttatggggcaggggtaagcaagtttttaaatcatttgtgtgctctggctcttttgatagaagaaagcaacacaaaagctccaaagggccccctaaccctcttgtggctccagttatttggaaactatgatctgcatccttaggaatctgggatttgccagttgctggcaatgtagagcaggcatggaattttatatgctagtgagtcataatgatatgttagtgttaattagttttttcttcctttgattttattggccataattgctactcttcatacacagtatatcaaagagcttgataatttagtt

SEQ ID NO: 117 - RNA, 23 nt, synthetic construct
  source 1..23: mol_type = other RNA
ttgctggacatttcaccatcagg

SEQ ID NO: 118 - RNA, 23 nt, synthetic construct
  source 1..23: mol_type = other RNA
tgctggacatttcaccatcaggg

SEQ ID NO: 119 - RNA, 23 nt, synthetic construct
  source 1..23: mol_type = other RNA
tccagcaaaagagtgcaatgagg

SEQ ID NO: 120 - RNA, 23 nt, synthetic construct
  source 1..23: mol_type = other RNA
aagcatggatatcggcaagaggg

SEQ ID NO: 121 - RNA, 23 nt, synthetic construct
  source 1..23: mol_type = other RNA
aagatgtatcttactgcagttgg

SEQ ID NO: 122 - RNA, 23 nt, synthetic construct
  source 1..23: mol_type = other RNA
cgaggaacgtgaccatggagtgg

SEQ ID NO: 123 - RNA, 23 nt, synthetic construct
  source 1..23: mol_type = other RNA
gtttagcagtgccccatgtgagg

SEQ ID NO: 124 - RNA, 23 nt, synthetic construct
  source 1..23: mol_type = other RNA
cttcctcttgagtccccgacggg

SEQ ID NO: 125 - RNA, 23 nt, synthetic construct
  source 1..23: mol_type = other RNA
atctgcaacactgcccgtcgggg

SEQ ID NO: 126 - RNA, 23 nt, synthetic construct
  source 1..23: mol_type = other RNA
tcgggaagtaaacctcacatggg

SEQ ID NO: 127 - RNA, 23 nt, synthetic construct
  source 1..23: mol_type = other RNA
catgtgaggtttacttcccgagg

SEQ ID NO: 128 - RNA, 23 nt, synthetic construct
  source 1..23: mol_type = other RNA
acatctgcaacactgcccgtcgg

SEQ ID NO: 129 - RNA, 23 nt, synthetic construct
  source 1..23: mol_type = other RNA
cgggaagtaaacctcacatgggg

SEQ ID NO: 130 - RNA, 23 nt, synthetic construct
  source 1..23: mol_type = other RNA
gtgcaatgaggaggtcagtttgg

SEQ ID NO: 131 - RNA, 19 nt, synthetic construct
  source 1..19: mol_type = other RNA
tcagatggcaatgtcgaga

SEQ ID NO: 132 - RNA, 18 nt, synthetic construct
  source 1..18: mol_type = other RNA
ttttccggatcgatgtga

SEQ ID NO: 133 - RNA, 19 nt, synthetic construct
  source 1..19: mol_type = other RNA
gacatctctgccgcatctg

SEQ ID NO: 134 - RNA, 19 nt, synthetic construct
  source 1..19: mol_type = other RNA
gtgggtgctgaatttcatc

SEQ ID NO: 135 - RNA, 19 nt, synthetic construct
  source 1..19: mol_type = other RNA
gattgtgggccaagtaacg

SEQ ID NO: 136 - RNA, 19 nt, synthetic construct
  source 1..19: mol_type = other RNA
gaaagtcactgttggtcga

SEQ ID NO: 137 - RNA, 19 nt, synthetic construct
  source 1..19: mol_type = other RNA
caattttgaggtgttcgtt

SEQ ID NO: 138 - RNA, 19 nt, synthetic construct
  source 1..19: mol_type = other RNA
gggttgagttcaacctaag

SEQ ID NO: 139 - RNA, 19 nt, synthetic construct
  source 1..19: mol_type = other RNA
ttagcctcattgtactagc

SEQ ID NO: 140 - RNA, 19 nt, synthetic construct
  source 1..19: mol_type = other RNA
gcaattttgaggtgttcgt

SEQ ID NO: 141 - RNA, 19 nt, synthetic construct
  source 1..19: mol_type = other RNA
accagcctcgggatctcaa

SEQ ID NO: 142 - RNA, 19 nt, synthetic construct
  source 1..19: mol_type = other RNA
tcaaatcagtcgggtttcc

SEQ ID NO: 143 - RNA, 23 nt, synthetic construct
  source 1..23: mol_type = other RNA
gagagtcctctatgcctaatggg

SEQ ID NO: 144 - RNA, 23 nt, synthetic construct
  source 1..23: mol_type = other RNA
aggggacccattaggcatagagg

SEQ ID NO: 145 - RNA, 23 nt, synthetic construct
  source 1..23: mol_type = other RNA
agagagtcctctatgcctaatgg

SEQ ID NO: 146 - RNA, 23 nt, synthetic construct
  source 1..23: mol_type = other RNA
aagccctcaatgcaacccagagg

SEQ ID NO: 147 - RNA, 23 nt, synthetic construct
  source 1..23: mol_type = other RNA
agccctcaatgcaacccagaggg

SEQ ID NO: 148 - RNA, 23 nt, synthetic construct
  source 1..23: mol_type = other RNA
tagggcaaccacttatgagttgg

SEQ ID NO: 149 - RNA, 20 nt, synthetic construct
  source 1..20: mol_type = other RNA
gtcagatggcaatgtcgaga

SEQ ID NO: 150 - RNA, 20 nt, synthetic construct
  source 1..20: mol_type = other RNA
tgtgggtgctgaatttcatc

SEQ ID NO: 151 - RNA, 20 nt, synthetic construct
  source 1..20: mol_type = other RNA
ggggttgagttcaacctaag

SEQ ID NO: 152 - protein, 94 aa, Rattus norvegicus
CPGRDRRTEEENFRKKEEHCPELPPGSAKRALPTSTSSSPQQKKKPLDGEYFTLKIRGRERFEMFRELNEALELKDARAAEESGDSRAHSSYPK

SEQ ID NO: 153 - DNA, 536 bp, synthetic construct
  source 1..536: mol_type = other DNA
ttcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttc

SEQ ID NO: 154 - DNA, 522 bp, synthetic construct
  source 1..522: mol_type = other DNA
ttcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgcaccat

SEQ ID NO: 155 - DNA, 500 bp, synthetic construct
  source 1..500: mol_type = other DNA
ccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtg

SEQ ID NO: 156 - DNA, 500 bp, synthetic construct
  source 1..500: mol_type = other DNA
gtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgt

SEQ ID NO: 157 - DNA, 1189 bp, synthetic construct
  source 1..1189: mol_type = other DNA
aggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagtt

SEQ ID NO: 158 - DNA, 2544 bp, synthetic construct
  source 1..2544: mol_type = other DNA
cccagaaacgtgaccatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctctgggaattgaggatagcctggaatcccaggacagcatggaattctga

SEQ ID NO: 159 - DNA, 2558 bp, synthetic construct
  source 1..2558: mol_type = other DNA
gcgaagtgtacttccccagaaacgtgaccatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctctgggaattgaggatagcctggaatcccaggacagcatggaattctga

SEQ ID NO: 160 - DNA, 3583 bp, synthetic construct
  source 1..3583: mol_type = other DNA
ttcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttccccagaaacgtgaccatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctctgggaattgaggatagcctggaatcccaggacagcatggaattctgataaccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgtccagcaaaagagtg

SEQ ID NO: 161 - DNA, 4223 bp, synthetic construct
  source 1..4223: mol_type = other DNA
ttcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgccccatgtgaggtttacttccccagaaacgtgaccatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctttaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagtt

SEQ ID NO: 162 - DNA, 3583 bp, synthetic construct
  source 1..3583: mol_type = other DNA
ttcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgcaccatgcgaagtgtacttccccagaaacgtgaccatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctctgggaattgaggatagcctggaatcccaggacagcatggaattctgataagtgaggtttacttcccgaggaacgtgaccatggagtggcacccccacacaccatcctgtgacatctgcaacactgcccgtcggggactcaagaggaagagtcttcagccaaacttgcagctcagcaaaaaactcaaaactgtgcttgaccaagcaagacaagcccgtcagcgcaagagaagagctcaggcaaggatcagcagcaaggatgtcatgaagaagatcgccaactgcagtaagatacatcttagtaccaagctccttgcagtggacttcccagagcactttgtgaaatccatctcctgccagatctgtgaacacattctggctgaccctgtggagaccaactgtaagcatgtcttttgccgggtctgcattctcagatgcctcaaagtcatgggcagctattgtccctcttgccgatatccatgcttccctactgacctggagagtccagtgaagtcctttctgagcgtcttgaattccctgatggtgaaatgt

SEQ ID NO: 163 - DNA, 4223 bp, synthetic construct
  source 1..4223: mol_type = other DNA
ttcagcacccacatattaaattttcagaatggaaatttaagctgttccgggtgagatcctttgaaaagacacctgaagaagctcaaaaggaaaagaaggattcctttgaggggaaaccctctctggagcaatctccagcagtcctggacaaggctgatggtcagaagccagtcccaactcagccattgttaaaagcccaccctaagttttcaaagaaatttcacgacaacgagaaagcaagaggcaaagcgatccatcaagccaaccttcgacatctctgccgcatctgtgggaattcttttagagctgatgagcacaacaggagatatccagtccatggtcctgtggatggtaaaaccctaggccttttacgaaagaaggaaaagagagctacttcctggccggacctcattgccaaggttttccggatcgatgtgaaggcagatgttgactcgatccaccccactgagttctgccataactgctggagcatcatgcacaggaagtttagcagtgcaccatgcgaagtgtacttccccagaaacgtgaccatggaatggcaccctcacacacccagctgcgacatctgcaacacagccagaagaggcctgaagcggaagtccctgcagcctaatctgcagctgagcaagaaactgaaaaccgtgctggaccaggccagacaggcccggcaaagaaagagaagggcccaagccagaatcagcagcaaggacgtgatgaagaagatcgccaactgcagcaagatccacctgagcaccaaactgctggccgtggacttccctgagcacttcgtgaagtccatcagctgccagatctgcgagcacatcctggccgatcctgtggaaacaaactgcaagcacgtgttctgcagagtgtgcatcctgcggtgcctgaaagtgatgggcagctactgcccctcctgcagatacccttgcttccccaccgatctggaaagccctgtgaagtccttcctgagcgtgctgaacagcctgatggtcaagtgccccgccaaagaatgcaacgaggaagtgtccctggaaaagtacaaccaccacatcagcagccacaaagagtccaaagaaatcttcgtgcacatcaacaaaggcggcagaccccggcagcatctgctgtctcttacaagacgggcccagaagcaccggctgagagaactgaagctgcaagtgaaggcctttgccgacaaagaggaaggcggcgacgtcaagagcgtgtgcatgaccctgtttctgctggccctgagagcccggaatgagcatagacaggccgatgagctggaagccatcatgcaaggcaaaggcagcggactgcagcctgctgtgtgtctggctatcagagtgaacaccttcctgtcctgcagccagtaccacaagatgtaccggaccgtgaaggccattaccggcagacagatcttccagcctctgcacgccctgagaaacgccgagaaagttctgctgcctggctaccaccacttcgagtggcagcctccactgaagaacgtgtccagcagcaccgacgtgggcatcatcgatggactgagcggactgtctagcagcgtggacgactaccccgtggacacaatcgccaagcggttcagatacgacagcgccctggtgtctgccctgatggacatggaagaggacatcctggaaggcatgcggagccaggacctggacgattacctgaacggccctttcaccgtggtggtcaaagaaagctgtgacggcatgggcgacgtgtccgagaaacacggatctggacctgtggtgccagagaaggccgtgcggttcagcttcaccatcatgaagatcactatcgcccacagcagccagaacgtgaaagtgttcgaggaagccaagcctaacagcgagctgtgctgcaagcctctgtgtctgatgctggccgacgagagcgatcacgagacactgaccgccattctgagccctctgatcgccgaacgggaagccatgaagtcctccgagctgatgctcgaactcggcggcatcctgagaaccttcaagttcatcttccgcggcaccggctacgacgagaagctcgttagagaggtggaaggcctggaagcctctggcagcgtgtacatctgcaccctgtgtgacgccaccagactggaagctagccagaacctggtgttccacagcatcaccagaagccacgccgaaaacctggaaagatacgaagtgtggcggagcaacccctaccacgagagcgtggaagaactgcgggatagagtgaagggcgtgtccgccaagcctttcatcgagacagtgcctagcatcgacgccctgcactgcgatattggcaacgccgccgaattctacaagatctttcagctggaaatcggcgaggtctacaagaaccccaacgcctctaaagaggaacggaagcgctggcaggccacactggataagcacctgagaaagaagatgaatctgaagcccatcatgaggatgaacggcaacttcgcccggaagctgatgaccaaagaaaccgtggatgccgtgtgcgagctgatcccctctgaggaaagacacgaggccctgcgggaactgatggacctgtacctgaagatgaagcccgtgtggcggtctagctgtcctgccaaagagtgccctgagtctctgtgccagtacagcttcaacagccagagattcgccgagctgctgtccaccaagttcaagtacagatacgagggcaagatcaccaactacttccacaagaccctggctcacgtgcccgagatcatcgagagagatggctctattggcgcctgggcctctgagggcaatgagtctggcaacaagctgttccggcggttccgcaagatgaacgccagacagagcaagtgctacgagatggaagatgtgctgaagcaccactggctgtacaccagcaagtacctgcagaaattcatgaacgcccacaacgccctcaagaccagcggctttaccatgaatcctcaggccagcctgggcgatcctttaggcatagaggactctctggaaagccaagattcaatggaattttaagtagggcaaccacttatgagttggtttttgcaattgagtttccctctgggttgcattgagggcttctcctagcaccctttactgctgtgtatggggcttcaccatccaagaggtggtaggttggagtaagatgctacagatgctctcaagtcaggaatagaaactgatgagctgattgcttgaggcttttagtgagttccgaaaagcaacaggaaaaatcagttatctgaaagctcagtaactcagaacaggagtaactgcaggggaccagagatgagcaaagatctgtgtgtgttggggagctgtcatgtaaatcaaagccaaggttgtcaaagaacagccagtgaggccaggaaagaaattggtcttgtggttttcatttttttcccccttgattgattatattttgtattgagatatgataagtgccttctatttcatttttgaataattcttcatttttataattttacatatcttggcttgctatataagattcaaaagagctttttaaatttttctaataatatcttacatttgtacagcatgatgacctttacaaagtgctctcaatgcatttacccattcgttatataaatatgttacatcaggacaactttgagaaaatcagtccttttttatgtttaaattatgtatctattgtaaccttcagagtttaggaggtcatctgctgtcatggatttttcaataatgaatttagaatacacctgttagctacagttagttattaaatcttctgataatatatgtttacttagctatcagaagccaagtatgattctttatttttactttttcatttcaagaaatttagagtttccaaatttagagcttctgcatacagtcttaaagccacagaggcttgtaaaaatataggttagcttgatgtctaaaaatatatttcatgtcttactgaaacattttgccagactttctccaaatgaaacctgaatcaatttttctaaatctaggtttcatagagtcctctcctctgcaatgtgttattctttctataatgatcagtttactttcagtggattcagaattgtgtagcaggataaccttgtatttttccatccgctaagtttagatggagtccaaacgcagtacagcagaagagtt

SEQ ID NO: 164 - protein, 847 aa, Homo sapiens
PRNVTMEWHPHTPSCDICNTARRGLKRKSLQPNLQLSKKLKTVLDQARQARQRKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 165 - protein, 851 aa, Homo sapiens
EVYFPRNVTMEWHPHTPSCDICNTARRGLKRKSLQPNLQLSKKLKTVLDQARQARQRKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 166 - RNA, 23 nt, synthetic construct
  source 1..23: mol_type = other RNA
aacatcttctgtcgctgactcgg

SEQ ID NO: 167 - RNA, 23 nt, synthetic construct
  source 1..23: mol_type = other RNA
gtcagatggcaatgtcgagatgg

SEQ ID NO: 168 - DNA, 20 bp, synthetic construct
  source 1..20: mol_type = other DNA
aagagagctacttcctggcc

SEQ ID NO: 169 - DNA, 20 bp, synthetic construct
  source 1..20: mol_type = other DNA
gcacacggacttcacatctc

SEQ ID NO: 170 - DNA, 20 bp, synthetic construct
  source 1..20: mol_type = other DNA
agccaaccttcgacatctct

SEQ ID NO: 171 - DNA, 20 bp, synthetic construct
  source 1..20: mol_type = other DNA
caaagtgctctgggaagtcc

SEQ ID NO: 172 - DNA, 20 bp, synthetic construct
  source 1..20: mol_type = other DNA
agtgccccatgtgaggttta

SEQ ID NO: 173 - DNA, 21 bp, synthetic construct
  source 1..21: mol_type = other DNA
catcagggaattcaagacgct

SEQ ID NO: 174 - DNA, 20 bp, synthetic construct
  source 1..20: mol_type = other DNA
aggatcagcagcaaggatgt

SEQ ID NO: 175 - DNA, 20 bp, synthetic construct
  source 1..20: mol_type = other DNA
gcacacggacttcacatctc

SEQ ID NO: 176 - DNA, 22 bp, synthetic construct
  source 1..22: mol_type = other DNA
gctgagctcctttctacgaagt

SEQ ID NO: 177 - DNA, 25 bp, synthetic construct
  source 1..25: mol_type = other DNA
gaaaaccacaagaccaatttctttc

SEQ ID NO: 178 - DNA, 19 bp, synthetic construct
  source 1..19: mol_type = other DNA
tccatgcttccctactgac

SEQ ID NO: 179 - DNA, 20 bp, synthetic construct
  source 1..20: mol_type = other DNA
ctcccattccatcacaagac

SEQ ID NO: 180 - DNA, 20 bp, synthetic construct
  source 1..20: mol_type = other DNA
gaagtggttcatgcaagagg

SEQ ID NO: 181 - DNA, 21 bp, synthetic construct
  source 1..21: mol_type = other DNA
ggatgaacatggagaaagcag

SEQ ID NO: 182 - DNA, 3083 bp, synthetic construct; identical to SEQ ID NO: 106
  source 1..3083: mol_type = other DNA; note = insert

SEQ ID NO: 183 - DNA, 19 bp, synthetic construct
  source 1..19: mol_type = other DNA
gccaatgtcaatgtcgaga

SEQ ID NO: 184 - DNA, 19 bp, synthetic construct
  source 1..19: mol_type = other DNA
gtcaaagtcaatgtcgaga

SEQ ID NO: 185 - DNA, 19 bp, synthetic construct
  source 1..19: mol_type = other DNA
atcagatggcaagtcgaga

SEQ ID NO: 186 - DNA, 20 bp, synthetic construct
  source 1..20: mol_type = other DNA
ttcagatggaaatgtcaagt

SEQ ID NO: 187 - DNA, 19 bp, synthetic construct
  source 1..19: mol_type = other DNA
gtaagatgcaatgtagaga

SEQ ID NO: 188 - DNA, 19 bp, synthetic construct
  source 1..19: mol_type = other DNA
gtcagatggcaatgtcaga

SEQ ID NO: 189 - DNA, 19 bp, synthetic construct
  source 1..19: mol_type = other DNA
atcagatcgcaagtcgaga

SEQ ID NO: 190 - DNA, 19 bp, synthetic construct
  source 1..19: mol_type = other DNA
gccagagccaatgtcgaga

SEQ ID NO: 191 - DNA, 20 bp, synthetic construct
  source 1..20: mol_type = other DNA
gacagctggcaatgttaaga

SEQ ID NO: 192 - DNA, 20 bp, synthetic construct
  source 1..20: mol_type = other DNA
ttcacatggcaatgtcaagt

SEQ ID NO: 193 - DNA, 2327 bp, synthetic construct; identical to SEQ ID NO: 17
  source 1..2327: mol_type = other DNA

SEQ ID NO: 194 - DNA, 4481 bp, synthetic construct; identical to SEQ ID NO: 107
  source 1..4481: mol_type = other DNA

SEQ ID NO: 195 - DNA, 2281 bp, synthetic construct; identical to SEQ ID NO: 18
  source 1..2281: mol_type = other DNA

SEQ ID NO: 196 - DNA, 1800 bp, synthetic construct; identical to SEQ ID NO: 79
  source 1..1800: mol_type = other DNA

SEQ ID NO: 197 - DNA, 3581 bp, synthetic construct; identical to SEQ ID NO: 108
  source 1..3581: mol_type = other DNA

SEQ ID NO: 198 - DNA, 2281 bp, synthetic construct; identical to SEQ ID NO: 18
  source 1..2281: mol_type = other DNA

SEQ ID NO: 199 - DNA, 900 bp, synthetic construct; identical to SEQ ID NO: 80
  source 1..900: mol_type = other DNA

SEQ ID NO: 200 - DNA, 3282 bp, synthetic construct; identical to SEQ ID NO: 109
  source 1..3282: mol_type = other DNA

SEQ ID NO: 201 - DNA, 503 bp, synthetic construct; identical to SEQ ID NO: 71
  source 1..503: mol_type = other DNA

SEQ ID NO: 202 - DNA, 2529 bp, synthetic construct; identical to SEQ ID NO: 19
  source 1..2529: mol_type = other DNA

SEQ ID NO: 203 - DNA, 4523 bp, synthetic construct; identical to SEQ ID NO: 110
  source 1..4523: mol_type = other DNA

SEQ ID NO: 204 - DNA, 2480 bp, synthetic construct; identical to SEQ ID NO: 20
  source 1..2480: mol_type = other DNA

SEQ ID NO: 205 - DNA, 1800 bp, synthetic construct; identical to SEQ ID NO: 79
  source 1..1800: mol_type = other DNA

SEQ ID NO: 206 - DNA, 3623 bp, synthetic construct; identical to SEQ ID NO: 111
  source 1..3623: mol_type = other DNA

SEQ ID NO: 207 - DNA, 2480 bp, synthetic construct; identical to SEQ ID NO: 20
  source 1..2480: mol_type = other DNA

SEQ ID NO: 208 - DNA, 900 bp, synthetic construct; identical to SEQ ID NO: 80
  source 1..900: mol_type = other DNA

SEQ ID NO: 209 - DNA, 4542 bp, synthetic construct; identical to SEQ ID NO: 112
  source 1..4542: mol_type = other DNA

SEQ ID NO: 210 - DNA, 2512 bp, synthetic construct; identical to SEQ ID NO: 21
  source 1..2512: mol_type = other DNA

SEQ ID NO: 211 - DNA, 1800 bp, synthetic construct; identical to SEQ ID NO: 79
  source 1..1800: mol_type = other DNA

SEQ ID NO: 212 - DNA, 4505 bp, synthetic construct; identical to SEQ ID NO: 113
  source 1..4505: mol_type = other DNA

SEQ ID NO: 213 - DNA, 2436 bp, synthetic construct; identical to SEQ ID NO: 22
  source 1..2436: mol_type = other DNA

SEQ ID NO: 214 - DNA, 1800 bp, synthetic construct; identical to SEQ ID NO: 79
  source 1..1800: mol_type = other DNA

SEQ ID NO: 215 - DNA, 4528 bp, synthetic construct; identical to SEQ ID NO: 114
  source 1..4528: mol_type = other DNA

SEQ ID NO: 216 - DNA, 2498 bp, synthetic construct; identical to SEQ ID NO: 23
  source 1..2498: mol_type = other DNA

SEQ ID NO: 217 - DNA, 1800 bp, synthetic construct; identical to SEQ ID NO: 79
  source 1..1800: mol_type = other DNA

SEQ ID NO: 218 - DNA, 4284 bp, synthetic construct; identical to SEQ ID NO: 115
  source 1..4284: mol_type = other DNA

SEQ ID NO: 219 - DNA, 500 bp, synthetic construct; identical to SEQ ID NO: 76
  source 1..500: mol_type = other DNA

SEQ ID NO: 220 - DNA, 1984 bp, synthetic construct; identical to SEQ ID NO: 24
  source 1..1984: mol_type = other DNA

SEQ ID NO: 221 - DNA, 1800 bp, synthetic construct; identical to SEQ ID NO: 79
  source 1..1800: mol_type = other DNA

SEQ ID NO: 222 - DNA, 5254 bp, synthetic construct; identical to SEQ ID NO: 116
  source 1..5254: mol_type = other DNA

SEQ ID NO: 223 - DNA, 26 bp, synthetic construct
  source 1..26: mol_type = other DNA
ctgacctcttctcttcctcccacagg

SEQ ID NO: 224 - DNA, 3132 bp, synthetic construct; identical to SEQ ID NO: 16
  source 1..3132: mol_type = other DNA

SEQ ID NO: 225 - DNA, 1792 bp, synthetic construct; identical to SEQ ID NO: 94
  source 1..1792: mol_type = other DNA

SEQ ID NO: 226 - RNA, 17 nt, synthetic construct
  source 1..17: mol_type = other RNA
ccttctcagcattccga

SEQ ID NO: 227 - RNA, 20 nt, synthetic construct
  source 1..20: mol_type = other RNA
aacatcttctgtcgctgact

SEQ ID NO: 228 - RNA, 20 nt, synthetic construct
  source 1..20: mol_type = other RNA
gtaccttctcagcattccga

SEQ ID NO: 229 - DNA, 22 bp, synthetic construct
  source 1..22: mol_type = other DNA
ccataaacactgtcagaagagg

SEQ ID NO: 230 - DNA, 19 bp, synthetic construct
  source 1..19: mol_type = other DNA
gtgttgcagatgtcacagg

SEQ ID NO: 231 - DNA, 21 bp, synthetic construct
  source 1..21: mol_type = other DNA
ggggagaaatgtgtagggaag

SEQ ID NO: 232 - DNA, 22 bp, synthetic construct
  source 1..22: mol_type = other DNA
ctcaaaaacaaagaaatgggcg

SEQ ID NO: 233 - DNA, 20 bp, synthetic construct
  source 1..20: mol_type = other DNA
ataggtggatgggatgatgg

SEQ ID NO: 234 - DNA, 22 bp, synthetic construct
  source 1..22: mol_type = other DNA
cctcttctgacagtgtttatgg

SEQ ID NO: 235 - DNA, 20 bp, synthetic construct
  source 1..20: mol_type = other DNA
ggaaaatgaatgccaggcag

SEQ ID NO: 236 - DNA, 22 bp, synthetic construct
  source 1..22: mol_type = other DNA
aggtcatcatgctgtacaaatg

SEQ ID NO: 237 - DNA, 24 bp, synthetic construct
  source 1..24: mol_type = other DNA
tcagaatggaaatttaagctgttc

SEQ ID NO: 238 - DNA, 23 bp, synthetic construct
  source 1..23: mol_type = other DNA
cacccaccttgggactcagttct

SEQ ID NO: 239 - DNA, 19 bp, synthetic construct
  source 1..19: mol_type = other DNA
tccgcttcaggcctcttct

SEQ ID NO: 240 - DNA, 3282 bp, synthetic construct; identical to SEQ ID NO: 109
  source 1..3282: mol_type = other DNA

SEQ ID NO: 241 - DNA, 2529 bp, synthetic construct; identical to SEQ ID NO: 19
  source 1..2529: mol_type = other DNA

SEQ ID NO: 242 - DNA, 4523 bp, synthetic construct; identical to SEQ ID NO: 110
  source 1..4523: mol_type = other DNA

SEQ ID NO: 243 - DNA, 2480 bp, synthetic construct; identical to SEQ ID NO: 20
  source 1..2480: mol_type = other DNA

SEQ ID NO: 244 - DNA, 1800 bp, synthetic construct; identical to SEQ ID NO: 79
  source 1..1800: mol_type = other DNA

SEQ ID NO: 245 - DNA, 3583 bp, synthetic construct; identical to SEQ ID NO: 160
  source 1..3583: mol_type = other DNA

SEQ ID NO: 246 - DNA, 2544 bp, synthetic construct; identical to SEQ ID NO: 158
  source 1..2544: mol_type = other DNA

SEQ ID NO: 247 - DNA, 4223 bp, synthetic construct; identical to SEQ ID NO: 161
  source 1..4223: mol_type = other DNA

SEQ ID NO: 248 - DNA, 2498 bp, synthetic construct; identical to SEQ ID NO: 23
  source 1..2498: mol_type = other DNA

SEQ ID NO: 249 - DNA, 1189 bp, synthetic construct; identical to SEQ ID NO: 157
  source 1..1189: mol_type = other DNA

SEQ ID NO: 250 - DNA, 3583 bp, synthetic construct; identical to SEQ ID NO: 162
  source 1..3583: mol_type = other DNA

SEQ ID NO: 251 - DNA, 2558 bp, synthetic construct; identical to SEQ ID NO: 159
  source 1..2558: mol_type = other DNA

SEQ ID NO: 252 - DNA, 500 bp, synthetic construct; identical to SEQ ID NO: 156
  source 1..500: mol_type = other DNA

SEQ ID NO: 253 - DNA, 4223 bp, synthetic construct; identical to SEQ ID NO: 163
  source 1..4223: mol_type = other DNA

SEQ ID NO: 254 - DNA, 2512 bp, synthetic construct; identical to SEQ ID NO: 21
  source 1..2512: mol_type = other DNA

SEQ ID NO: 255 - DNA, 1189 bp, synthetic construct; identical to SEQ ID NO: 157
  source 1..1189: mol_type = other DNA

SEQ ID NO: 256 - DNA, 3623 bp, synthetic construct; identical to SEQ ID NO: 111
  source 1..3623: mol_type = other DNA

SEQ ID NO: 257 - DNA, 2480 bp, synthetic construct; identical to SEQ ID NO: 20
  source 1..2480: mol_type = other DNA

SEQ ID NO: 258 - DNA, 900 bp, synthetic construct; identical to SEQ ID NO: 80
  source 1..900: mol_type = other DNA

(19) United States
(12) Patent Application Publication
(10) Pub. No.: US 2024/0425851 A1
(43) Pub. Date: Dec. 26, 2024

(54) POLYNUCLEOTIDES USEFUL FOR CORRECTING MUTATIONS IN THE RAG1 GENE

(71) Applicants: OSPEDALE SAN RAFFAELE S.R.L. (50%), Milan (IT); FONDAZIONE TELETHON ETS (50%), Rome (IT)

(72) Inventors: Anna Villa, Milan (IT); Luigi Naldini, Milan (IT); Samuele Ferrari, Milan (IT); Maria Carmina Castiello, Milan (IT); Simona Porcellini, Milan (IT); Daniele Canarutto, Milan (IT)

(73) Assignees: OSPEDALE SAN RAFFAELE S.R.L. (50%), Milan (IT); FONDAZIONE TELETHON ETS (50%), Rome (IT)

(21) Appl. No.: 18/700,795

(22) PCT Filed: Oct. 11, 2022
(86) PCT No.: PCT/EP2022/078298

§ 371 Date: Apr. 12, 2024

(30) Foreign Application Priority Data
Oct. 12, 2021 (GB) 2114587.5
Apr. 14, 2022 (GB) 2205593.3

(51) Int. Cl. C12N 15/11; C12N 9/22; C12N 15/90

(52) U.S. Cl. CPC C12N 15/11; C12N 9/22; C12N 15/907; C12N 2310/20; C12N 2310/315

(57) ABSTRACT

The present invention relates to an isolated polynucleotide comprising from 5′ to 3′: a first homology region, a nucleotide sequence encoding a RAG1 polypeptide or a RAG1 polypeptide fragment, and a second homology region for use in treating a RAG-deficient immunodeficiency.

### FIELD OF THE INVENTION

The present invention relates to methods for gene-editing cells to introduce a RAG1 polypeptide or a RAG1 polypeptide fragment, for example as a treatment for severe combined immunodeficiency. The present invention also relates to polynucleotides, vectors, guide RNAs, kits, compositions, and gene editing systems for use in said methods. The present invention also relates to genomes and cells obtained or obtainable by said methods.

### BACKGROUND TO THE INVENTION

The RAG1 and RAG2 proteins initiate V(D)J recombination, allowing generation of a diverse repertoire of T and B cells (Teng G, Schatz D G. Advances in Immunology. 2015; 128:1-39). RAG mutations in humans cause a broad spectrum of phenotypes, including T⁻ B⁻ SCID, Omenn syndrome (OS), atypical SCID (AS) and combined immunodeficiency with granuloma/autoimmunity (CID-G/AI) (Notarangelo L D, et al. Nat Rev Immunol. 2016; 16(4):234-246).

Hematopoietic stem cell transplantation (HSCT) is the mainstay for severe forms of RAG1 deficiency, including T⁻ B⁻ SCID, OS and AS with an overall survival of ˜80% after transplantation from donors other than matched siblings (Haddad E, et al. Blood. 2018; 132(17):1737-49). However, overall survival rate is lower in non-matched-sibling donors and a high rate of graft failure and poor T and B cell immune reconstitution are observed in the absence of myeloablative or reduced intensity conditioning. Besides donor type and conditioning, other factors associated with worse outcomes after HSCT include age (>3.5 months of life) and infections at the time of transplantation.

An alternative approach to overcome the obstacles with HSCT is represented by gene therapy. Selective advantage of gene-corrected hematopoietic stem cells (HSCs) to overcome the block of T and B cells that occur in the absence of RAG activity represents the rationale for developing such a strategy. In recent years, lentiviral vectors have become the strategy of choice to deliver the transgene of interest, and allow its expression under the control of suitable promoters (Naldini L, Nature. 2015; 526:351-360). In the case of RAG1 deficiency, the observation that endogenous RAG1 gene expression is tightly regulated during cell cycle and during lymphoid development, may expose to the risk that ectopic or dysregulated gene expression could lead to immune dysregulation or leukemia (Lagresle-Peyrou C, et al. Blood. 2006; 107(1):63-72; Pike-Overzet K, et al. Leukemia. 2011; 25(9):1471-83; and Pike-Overzet K, et al. Journal of Allergy and Clinical Immunology. 2014; 134:242-243). Several groups have examined the safety and efficacy of lentivirus-mediated gene therapy for RAG deficiency in preclinical models showing poor immune reconstitution or severe signs of inflammation, with cellular infiltrates in the skin, lung, liver, kidney, and presence of circulating anti-double strand DNA (van Til N P, et al. J Allergy Clin Immunol. 2014; 133(4):1116-23).

Overall, these data raise significant concerns on the clinical use of conventional RAG1 gene therapy vectors that allow suboptimal levels and deregulated pattern of gene expression.

Thus, there is a demand for improved treatments for RAG1 deficiency.

### SUMMARY OF THE INVENTION

The present inventors have developed gene editing strategies to correct mutations in the RAG1 gene at the endogenous locus by introducing nucleotide sequence inserts encoding a RAG1 polypeptide or a RAG1 polypeptide fragment.

The present inventors have developed a gene editing strategy to correct mutations in the RAG1 gene at the endogenous locus by targeting the second exon, which contains the entire coding sequence of the gene. The present inventors have also developed a gene editing strategy to correct mutations in the RAG1 gene at the endogenous locus by targeting the first intron or the start of the second exon.

The present inventors have designed and selected a panel of CRISPR-Cas9 nucleases and corrective donors for these strategies.

The present invention provides a polynucleotide comprising from 5′ to 3′: a first homology region, a nucleotide sequence encoding a RAG1 polypeptide or a RAG1 polypeptide fragment, and a second homology region.

In a first aspect, the present invention provides a polynucleotide comprising from 5′ to 3′: a first homology region, a nucleotide sequence encoding a RAG1 polypeptide fragment, and a second homology region, wherein the first homology region is homologous to a first region of the RAG1 exon 2 and the second homology region is homologous to a second region of the RAG1 exon 2.

In some embodiments:
(i) the first homology region is homologous to a region upstream of chr 11:36574368 and the second homology region is homologous to a region downstream of chr 11:36574369;
(ii) the first homology region is homologous to a region upstream of chr 11:36574367 and the second homology region is homologous to a region downstream of chr 11:36574368;
(iii) the first homology region is homologous to a region upstream of chr 11:36574394 and the second homology region is homologous to a region downstream of chr 11:36574395;
(iv) the first homology region is homologous to a region upstream of chr 11:36574294 and the second homology region is homologous to a region downstream of chr 11:36574295;
(v) the first homology region is homologous to a region upstream of chr 11:36574109 and the second homology region is homologous to a region downstream of chr 11:36574110;
(vi) the first homology region is homologous to a region upstream of chr 11:36573910 and the second homology region is homologous to a region downstream of chr 11:36573911;
(vii) the first homology region is homologous to a region upstream of chr 11:36573878 and the second homology region is homologous to a region downstream of chr 11:36573879;
(viii) the first homology region is homologous to a region upstream of chr 11:36573959 and the second homology region is homologous to a region downstream of chr 11:36573960;
(ix) the first homology region is homologous to a region upstream of chr 11:36573957 and the second homology region is homologous to a region downstream of chr 11:36573958;
(x) the first homology region is homologous to a region upstream of chr 11:36573879 and the second homology region is homologous to a region downstream of chr 11:36573880;
(xi) the first homology region is homologous to a region upstream of chr 11:36573892 and the second homology region is homologous to a region downstream of chr 11:36573893;
(xii) the first homology region is homologous to a region upstream of chr 11:36573955 and the second homology region is homologous to a region downstream of chr 11:36573956;
(xiii) the first homology region is homologous to a region upstream of chr 11:36573878 and the second homology region is homologous to a region downstream of chr 11:36573879; or
(xiv) the first homology region is homologous to a region upstream of chr 11:36574406 and the second homology region is homologous to a region downstream of chr 11:36574407.

In some embodiments:
(v) the first homology region is homologous to a region upstream of chr 11:36574109 and the second homology region is homologous to a region downstream of chr 11:36574110; or
(vi) the first homology region is homologous to a region upstream of chr 11:36573910 and the second homology region is homologous to a region downstream of chr 11:36573911.

In some embodiments:
(xi) the first homology region is homologous to a region upstream of chr 11:36573892 and the second homology region is homologous to a region downstream of chr 11:36573893; or
(xiii) the first homology region is homologous to a region upstream of chr 11:36573878 and the second homology region is homologous to a region downstream of chr 11:36573879.

In some embodiments, the first homology region is homologous to a region upstream of chr 11:36573878 and the second homology region is homologous to a region downstream of chr 11:36573879.

In some embodiments:
(i) the first homology region is homologous to a region comprising chr 11:36574319-36574368;
(ii) the first homology region is homologous to a region comprising chr 11:36574318-36574367;
(iii) the first homology region is homologous to a region comprising chr 11:36574345-36574394;
(iv) the first homology region is homologous to a region comprising chr 11:36574245-36574294;
(v) the first homology region is homologous to a region comprising chr 11:36574060-36574109;
(vi) the first homology region is homologous to a region comprising chr 11:36573861-36573910;
(vii) the first homology region is homologous to a region comprising chr 11:36573829-36573878;
(viii) the first homology region is homologous to a region comprising chr 11:36573910-36573959;
(ix) the first homology region is homologous to a region comprising chr 11:36573908-36573957;
(x) the first homology region is homologous to a region comprising chr 11:36573830-36573879;
(xi) the first homology region is homologous to a region comprising chr 11:36573843-36573892;
(xii) the first homology region is homologous to a region comprising chr 11:36573906-36573955;
(xiii) the first homology region is homologous to a region comprising chr 11:36573829-36573878; or
(xiv) the first homology region is homologous to a region comprising chr 11:36574357-36574406.

In some embodiments:
(i) the first homology region is homologous to a region comprising chr 11:36574319-36574368 and/or the second homology region is homologous to a region comprising chr 11:36574369-36574418;
(ii) the first homology region is homologous to a region comprising chr 11:36574318-36574367 and/or the second homology region is homologous to a region comprising chr 11:36574368-36574417;
(iii) the first homology region is homologous to a region comprising chr 11:36574345-36574394 and/or the second homology region is homologous to a region comprising chr 11:36574395-36574444;
(iv) the first homology region is homologous to a region comprising chr 11:36574245-36574294 and/or the second homology region is homologous to a region comprising chr 11:36574295-36574344;
(v) the first homology region is homologous to a region comprising chr 11:36574060-36574109 and/or the second homology region is homologous to a region comprising chr 11:36574110-36574159;
(vi) the first homology region is homologous to a region comprising chr 11:36573861-36573910 and/or the second homology region is homologous to a region comprising chr 11:36573911-36573960;
(vii) the first homology region is homologous to a region comprising chr 11:36573829-36573878 and/or the second homology region is homologous to a region comprising chr 11:36573879-36573928;
(viii) the first homology region is homologous to a region comprising chr 11:36573910-36573959 and/or the second homology region is homologous to a region comprising chr 11:36573960-36574009;
(ix) the first homology region is homologous to a region comprising chr 11:36573908-36573957 and/or the second homology region is homologous to a region comprising chr 11:36573958-36574007;
(x) the first homology region is homologous to a region comprising chr 11:36573830-36573879 and/or the second homology region is homologous to a region comprising chr 11:36573880-36573929;
(xi) the first homology region is homologous to a region comprising chr 11:36573843-36573892 and/or the second homology region is homologous to a region comprising chr 11:36573893-36573942;
(xii) the first homology region is homologous to a region comprising chr 11:36573906-36573955 and/or the second homology region is homologous to a region comprising chr 11:36573956-36574005;
(xiii) the first homology region is homologous to a region comprising chr 11:36573829-36573878 and/or the second homology region is homologous to a region comprising chr 11:36573879-36573928; or
(xiv) the first homology region is homologous to a region comprising chr 11:36574357-36574406 and/or the second homology region is homologous to a region comprising chr 11:36574407-36574456.

In some embodiments:
(i) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 25;
(ii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 26;
(iii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 27;
(iv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 28;
(v) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39;
(vi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40;
(vii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 31 or SEQ ID NO: 41;
(viii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 32 or SEQ ID NO: 42;
(ix) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 33 or SEQ ID NO: 42;
(x) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 34 or SEQ ID NO: 41;
(xi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41;
(xii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 36 or SEQ ID NO: 42;
(xiii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43; or
(xiv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 38 or SEQ ID NO: 44.

In some embodiments:
(i) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 25 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 45;
(ii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 26 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 46;
(iii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 27 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 47;
(iv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 28 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 48;
(v) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 49 or SEQ ID NO: 59;
(vi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 50 or SEQ ID NO: 60;
(vii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 31 or SEQ ID NO: 41 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 51;
(viii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 32 or SEQ ID NO: 42 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 52;
(ix) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 33 or SEQ ID NO: 42 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 53;
(x) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 34 or SEQ ID NO: 41 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 54;
(xi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 55;
(xii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 36 or SEQ ID NO: 42 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 56;
(xiii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 57; or
(xiv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 38 or SEQ ID NO: 44 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 58.

In some embodiments:
(1) the first homology region comprises or consists of a nucleotide sequence that has at least 70% identity, at least 80% identity, at least 90% identity, at least 95% identity, at least 98% identity, or 100% identity to SEQ ID NO: 69, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 77, or a fragment thereof; or
(4) the first homology region comprises or consists of a nucleotide sequence that has at least 70% identity, at least 80% identity, at least 90% identity, at least 95% identity, at least 98% identity, or 100% identity to SEQ ID NO: 71, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 78, or a fragment thereof.

In some embodiments:
(5) the first homology region comprises or consists of a nucleotide sequence that has at least 70% identity, at least 80% identity, at least 90% identity, at least 95% identity, at least 98% identity, or 100% identity to SEQ ID NO: 72, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof; or
(6) the first homology region comprises or consists of a nucleotide sequence that has at least 70% identity, at least 80% identity, at least 90% identity, at least 95% identity, at least 98% identity, or 100% identity to SEQ ID NO: 72, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 80, or a fragment thereof.

In some embodiments:
(12) the first homology region comprises or consists of a nucleotide sequence that has at least 70% identity, at least 80% identity, at least 90% identity, at least 95% identity, at least 98% identity, or 100% identity to SEQ ID NO: 153, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 155, or a fragment thereof;
(13) the first homology region comprises or consists of a nucleotide sequence that has at least 70% identity, at least 80% identity, at least 90% identity, at least 95% identity, at least 98% identity, or 100% identity to SEQ ID NO: 153, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 157, or a fragment thereof;
(14) the first homology region comprises or consists of a nucleotide sequence that has at least 70% identity, at least 80% identity, at least 90% identity, at least 95% identity, at least 98% identity, or 100% identity to SEQ ID NO: 154, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 156, or a fragment thereof; or
(15) the first homology region comprises or consists of a nucleotide sequence that has at least 70% identity, at least 80% identity, at least 90% identity, at least 95% identity, at least 98% identity, or 100% identity to SEQ ID NO: 154, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 157, or a fragment thereof.

In some embodiments:
(14) the first homology region comprises or consists of a nucleotide sequence that has at least 70% identity, at least 80% identity, at least 90% identity, at least 95% identity, at least 98% identity, or 100% identity to SEQ ID NO: 154, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 156, or a fragment thereof; or
(15) the first homology region comprises or consists of a nucleotide sequence that has at least 70% identity, at least 80% identity, at least 90% identity, at least 95% identity, at least 98% identity, or 100% identity to SEQ ID NO: 154, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 157, or a fragment thereof.

The first and second homology regions may each be 50-2000 bp in length, 50-1800 bp in length, 50-1500 bp in length, 50-1000 bp in length, 100-500 bp in length, or 200-400 bp in length.

In a second aspect, the present invention provides a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a first region of the RAG1 intron 1 or exon 2 and the second homology region is homologous to a second region of the RAG1 exon 2.

In some embodiments, the splice acceptor site comprises or consists of a nucleotide sequence that has at least 70% identity to SEQ ID NO: 95.

In some embodiments, the first homology region is homologous to a region upstream of: (i) chr 11:36569295; (ii) chr 11:36573790; (iii) chr 11:36573641; (iv) chr 11:36573351; (v) chr 11:36569080; (vi) chr 11:36572472; (vii) chr 11:36571458; (viii) chr 11:36571366; (ix) chr 11:36572859 (x) chr 11:36571457; (xi) chr 11:36569351; or (xii) chr 11:36572375.

In some embodiments, the first homology region is homologous to a region upstream of: (i) chr 11:36569295; (ii) chr 11:36573351; (iii) chr 11:36571366, preferably wherein the first homology region is homologous to a region upstream of chr 11:36569295.

In some embodiments, the first homology region is homologous to a region comprising chr 11:36569245-chr 11:36569294, preferably wherein the 3′ terminal sequence of the first homology region comprises or consists of a nucleotide sequence that has at least 70% identity to SEQ ID NO: 81, more preferably wherein the first homology region comprises or consists of a nucleotide sequence that has at least 70% identity to SEQ ID NO: 93.

In some embodiments, the second homology region is downstream of chr 11:36574557; downstream of chr 11:36574870; downstream of chr 11:36575183; downstream of chr 11:36575496; downstream of chr 11:36575810; downstream of chr 11:36576123; or downstream of chr 11:36576436.

In some embodiments, the second homology region is homologous to a region comprising chr 11:36576437-chr 11:36576536.

In some embodiments:
(i) the first homology region is homologous to a region comprising chr 11:36574319-36574368 and/or the second homology region is homologous to a region comprising chr 11:36576437-36576536;
(ii) the first homology region is homologous to a region comprising chr 11:36574318-36574367 and/or the second homology region is homologous to a region comprising chr 11:36576437-36576536;
(iii) the first homology region is homologous to a region comprising chr 11:36574345-36574394 and/or the second homology region is homologous to a region comprising chr 11:36576437-36576536;
(iv) the first homology region is homologous to a region comprising chr 11:36574245-36574294 and/or the second homology region is homologous to a region comprising chr 11:36576437-36576536;
(v) the first homology region is homologous to a region comprising chr 11:36574060-36574109 and/or the second homology region is homologous to a region comprising chr 11:36576437-36576536;
(vi) the first homology region is homologous to a region comprising chr 11:36573861-36573910 and/or the second homology region is homologous to a region comprising chr 11:36576437-36576536;
(vii) the first homology region is homologous to a region comprising chr 11:36573829-36573878 and/or the second homology region is homologous to a region comprising chr 11:36576437-36576536;
(viii) the first homology region is homologous to a region comprising chr 11:36573910-36573959 and/or the second homology region is homologous to a region comprising chr 11:36576437-36576536;
(ix) the first homology region is homologous to a region comprising chr 11:36573908-36573957 and/or the second homology region is homologous to a region comprising chr 11:36576437-36576536;
(x) the first homology region is homologous to a region comprising chr 11:36573830-36573879 and/or the second homology region is homologous to a region comprising chr 11:36576437-36576536;
(xi) the first homology region is homologous to a region comprising chr 11:36573843-36573892 and/or the second homology region is homologous to a region comprising chr 11:36576437-36576536;
(xii) the first homology region is homologous to a region comprising chr 11:36573906-36573955 and/or the second homology region is homologous to a region comprising chr 11:36576437-36576536;
(xiii) the first homology region is homologous to a region comprising chr 11:36573829-36573878 and/or the second homology region is homologous to a region comprising chr 11:36576437-36576536; or
(xiv) the first homology region is homologous to a region comprising chr 11:36574357-36574406 and/or the second homology region is homologous to a region comprising chr 11:36576437-36576536.

In some embodiments, the second homology region comprises or consists of a nucleotide sequence that has at least 70% identity, at least 80% identity, at least 90% identity, at least 95% identity, at least 98% identity, or 100% identity to any of SEQ ID NOs: 79-80, 94 or 157, or a fragment thereof.

In some embodiments, the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67. In some embodiments:
(i) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 25 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(ii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 26 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(iii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 27 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(iv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 28 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(v) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(vi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(vii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 31 or SEQ ID NO: 41 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(viii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 32 or SEQ ID NO: 42 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(ix) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 33 or SEQ ID NO: 42 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(x) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 34 or SEQ ID NO: 41 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(xi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(xii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 36 or SEQ ID NO: 42 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(xiii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67; or
(xiv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 38 or SEQ ID NO: 44 and/or the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67.

In some embodiments:
(2) the first homology region comprises or consists of a nucleotide sequence that has at least 70% identity, at least 80% identity, at least 90% identity, at least 95% identity, at least 98% identity, or 100% identity to SEQ ID NO: 70, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof;
(3) the first homology region comprises or consists of a nucleotide sequence that has at least 70% identity, at least 80% identity, at least 90% identity, at least 95% identity, at least 98% identity, or 100% identity to SEQ ID NO: 70, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 80, or a fragment thereof;
(5) the first homology region comprises or consists of a nucleotide sequence that has at least 70% identity, at least 80% identity, at least 90% identity, at least 95% identity, at least 98% identity, or 100% identity to SEQ ID NO: 72, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof;
(6) the first homology region comprises or consists of a nucleotide sequence that has at least 70% identity, at least 80% identity, at least 90% identity, at least 95% identity, at least 98% identity, or 100% identity to SEQ ID NO: 72, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 80, or a fragment thereof;
(7) the first homology region comprises or consists of a nucleotide sequence that has at least 70% identity, at least 80% identity, at least 90% identity, at least 95% identity, at least 98% identity, or 100% identity to SEQ ID NO: 73, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof;
(8) the first homology region comprises or consists of a nucleotide sequence that has at least 70% identity, at least 80% identity, at least 90% identity, at least 95% identity, at least 98% identity, or 100% identity to SEQ ID NO: 74, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof;
(9) the first homology region comprises or consists of a nucleotide sequence that has at least 70% identity, at least 80% identity, at least 90% identity, at least 95% identity, at least 98% identity, or 100% identity to SEQ ID NO: 75, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof; or
(10) the first homology region comprises or consists of a nucleotide sequence that has at least 70% identity, at least 80% identity, at least 90% identity, at least 95% identity, at least 98% identity, or 100% identity to SEQ ID NO: 76, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof.

In some embodiments:
(11) the first homology region comprises or consists of a nucleotide sequence that has at least 70% identity, at least 80% identity, at least 90% identity, at least 95% identity, at least 98% identity, or 100% identity to SEQ ID NO: 93, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 94, or a fragment thereof.

In some embodiments, the first homology region is about 50-1000 bp in length, 100-500 bp in length, or 200-400 bp in length; and/or wherein the second homology region is about 500-2000 bp in length, 1000-2000 bp in length, or 1500-2000 bp in length.

In some embodiments, the nucleotide sequence encoding a RAG1 polypeptide comprises or consists of a nucleotide sequence encoding an amino acid sequence that has at least 70% identity to SEQ ID NO: 4, SEQ ID NO: 5, or SEQ ID NO: 6.

In some embodiments, the nucleotide sequence encoding a RAG1 polypeptide comprises or consists of a nucleotide sequence that has at least 70% identity to SEQ ID NO: 15.

In some embodiments, the nucleotide sequence encoding a RAG1 polypeptide fragment comprises or consists of a nucleotide sequence encoding a fragment of an amino acid sequence that has at least 70% identity, at least 80% identity, at least 90% identity, at least 95% identity, at least 98% identity, or 100% identity to SEQ ID NO: 4, SEQ ID NO: 5, or SEQ ID NO: 6.

In some embodiments, the RAG1 polypeptide fragment is at least 500 amino acids in length, at least 550 amino acids in length, at least 600 amino acids in length, at least 650 amino acids in length, at least 700 amino acids in length, at least 750 amino acids in length, or at least 800 amino acids in length.

In some embodiments, the RAG1 polypeptide fragment comprises or consists of an amino acid sequence that has at least 70% identity, at least 80% identity, at least 90% identity, at least 95% identity, at least 98% identity, or 100% identity to any one of SEQ ID NOs: 7 to 14, 164 or 165.

In some embodiments, the nucleotide sequence encoding a RAG1 polypeptide fragment comprises or consists of a fragment of a nucleotide sequence that has at least 70% identity, at least 80% identity, at least 90% identity, at least 95% identity, at least 98% identity, or 100% identity to SEQ ID NO: 15.

In some embodiments, the nucleotide sequence encoding a RAG1 polypeptide fragment is at least 1500 bp in length, at least 1600 bp in length, at least 1700 bp in length, at least 1800 bp in length, at least 1900 bp in length, at least 2000 bp in length, at least 2100 bp in length, at least 2200 bp in length, at least 2300 bp in length, or at least 2400 bp in length.

In some embodiments, the nucleotide sequence encoding a RAG1 polypeptide fragment comprises or consists of a nucleotide sequence that has at least 70% identity, at least 80% identity, at least 90% identity, at least 95% identity, at least 98% identity, or 100% identity to any one of SEQ ID NOs: 17 to 24, 158 or 159.

In some embodiments, the polynucleotide comprises or consists of a nucleotide sequence that has at least 70% identity to any one of SEQ ID NOs: 106 to 115 or 160 to 163. In some embodiments, the polynucleotide comprises or consists of a nucleotide sequence that has at least 70% identity, at least 80% identity, at least 90% identity, at least 95% identity, at least 98% identity, or 100% identity to SEQ ID NO: 116.

In another aspect, the present invention provides a vector comprising the polynucleotide of the invention.

In some embodiments, the vector is a viral vector, optionally an adeno-associated viral (AAV) vector such as an AAV6 vector. In some embodiments, the vector is a lentiviral vector, such as an integration-defective lentiviral vector (IDLV).

In another aspect, the present invention provides a guide RNA comprising or consisting of a nucleotide sequence that has at least 90% identity to any of SEQ ID NOs: 117-130.

In preferred embodiments, the guide RNA comprises or consists of a nucleotide sequence that has at least 90% identity to SEQ ID NO: 121. In preferred embodiments, the guide RNA comprises or consists of a nucleotide sequence that has at least 90% identity to SEQ ID NO: 122. In some embodiments, the guide RNA comprises or consists of a nucleotide sequence that has at least 90% identity to SEQ ID NO: 117. In some embodiments, the guide RNA comprises or consists of a nucleotide sequence that has at least 90% identity to SEQ ID NO: 118. In some embodiments, the guide RNA comprises or consists of a nucleotide sequence that has at least 90% identity to SEQ ID NO: 119. In some embodiments, the guide RNA comprises or consists of a nucleotide sequence that has at least 90% identity to SEQ ID NO: 120. In some embodiments, the guide RNA comprises or consists of a nucleotide sequence that has at least 90% identity to SEQ ID NO: 123. In some embodiments, the guide RNA comprises or consists of a nucleotide sequence that has at least 90% identity to SEQ ID NO: 124. In some embodiments, the guide RNA comprises or consists of a nucleotide sequence that has at least 90% identity to SEQ ID NO: 125. In some embodiments, the guide RNA comprises or consists of a nucleotide sequence that has at least 90% identity to SEQ ID NO: 126. In some embodiments, the guide RNA comprises or consists of a nucleotide sequence that has at least 90% identity to SEQ ID NO: 127. In some embodiments, the guide RNA comprises or consists of a nucleotide sequence that has at least 90% identity to SEQ ID NO: 128. In some embodiments, the guide RNA comprises or consists of a nucleotide sequence that has at least 90% identity to SEQ ID NO: 129. In some embodiments, the guide RNA comprises or consists of a nucleotide sequence that has at least 90% identity to SEQ ID NO: 130.

In another aspect, the present invention provides a guide RNA comprising or consisting of a nucleotide sequence that has at least 90% identity to any of SEQ ID NOs: 143-148.

In some embodiments, the guide RNA comprises or consists of a nucleotide sequence that has at least 90% identity to SEQ ID NO: 143. In some embodiments, the guide RNA comprises or consists of a nucleotide sequence that has at least 90% identity to SEQ ID NO: 144. In some embodiments, the guide RNA comprises or consists of a nucleotide sequence that has at least 90% identity to SEQ ID NO: 145. In some embodiments, the guide RNA comprises or consists of a nucleotide sequence that has at least 90% identity to SEQ ID NO: 146. In some embodiments, the guide RNA comprises or consists of a nucleotide sequence that has at least 90% identity to SEQ ID NO: 147. In some embodiments, the guide RNA comprises or consists of a nucleotide sequence that has at least 90% identity to SEQ ID NO: 148.

In some embodiments, from one to five of the terminal nucleotides at 5′ end and/or 3′ end of the guide RNA are chemically modified to enhance stability, optionally wherein three terminal nucleotides at 5′ end and/or 3′ end if the guide RNA are chemically modified to enhance stability, optionally wherein the chemical modification is modification with 2′-O-methyl 3′phosphorothioate.

In another aspect, the present invention provides a kit comprising the polynucleotide or the vector of the invention.

In another aspect, the present invention provides a composition comprising the polynucleotide or the vector of the invention.

In another aspect, the present invention provides a gene-editing system comprising the polynucleotide or the vector of the invention.

In some embodiments, the kit, composition, or gene-editing system further comprises a guide RNA of the invention. In some embodiments, the kit, composition, or gene-editing system further comprises a RNA-guided nuclease, optionally wherein the RNA-guided nuclease is a Cas9 endonuclease

In another aspect, the present invention provides for use of the polynucleotide, the vector, the kit, the composition, or the gene-editing system, for gene editing a cell or a population of cells. In some embodiments, the use is ex vivo or in vitro use.

In another aspect, the present invention provides a genome comprising the polynucleotide of the invention.

In another aspect, the present invention provides a cell comprising the polynucleotide, the vector, or the genome of the invention.

In another aspect, the present invention provides a population of cells comprising one or more cells of the present invention.

In another aspect, the present invention provides a method of gene editing a population of cells comprising delivering the polynucleotide or the vector of the invention to a population of cells to obtain a population of gene-edited cells. In some embodiments, the method is an ex vivo or in vitro method.

In another aspect, the present invention provides a method of treating immunodeficiency in a subject in need thereof, comprising delivering the polynucleotide or the vector of the invention to a population of cells to obtain a population of gene-edited cells and administering the population of gene-edited cells to the subject.

In another aspect, the present invention provides a population of gene-edited cells obtainable by the method of the invention.

In another aspect, the present invention provides the polynucleotide, the vector, the guide RNA, the kit, the composition, or the gene-editing system, for use in treating immunodeficiency in a subject.

In another aspect, the present invention provides a method of treating a subject comprising administering a cell, a population of cells, or a population of gene edited cells of the present invention to the subject.

In another aspect, the present invention provides a method of treating immunodeficiency in a subject in need thereof comprising administering a cell, a population of cells, or a population of gene edited cells of the present invention to the subject.

In another aspect, the present invention provides a cell, a population of cells, or a population of gene edited cells of the present invention for use as a medicament.

In another aspect, the present invention provides a cell, a population of cells, or a population of gene edited cells of the present invention for use in treating immunodeficiency in a subject.

### DESCRIPTION OF DRAWINGS

FIG. 1. RAG1 gene editing strategies

Schematic representation of two RAG1 gene editing strategies: (A) the “exon 2 RAG1 gene targeting” strategy and (B) the “exon 2 RAG1 gene replacement” strategy. (C) Schematic representations of RAG1 gene, protein domains and gRNA positions mapping at the 5′ region of RAG1 exon 2 (C). Guide RNAs shown in the box are specific for the exon 2 RAG1 gene targeting and replacement strategies. (D) The box highlights the positions of gRNAs targeting the 3′ region of RAG1 exon 2 which can be optionally combined with gRNA targeting the 5′ region of RAG1 exon 2 or gRNA targeting the intron 1 for the exonic and intronic replacement strategies, respectively. (A) Abbreviations: HA, homology arm; coRAG1 CDS, codon optimized RAG1 coding sequence; Ex., exon; gRNA, guide RNA; 3′UTR, 3′ untranslated region; HDR, homology directed repair. (C-D) Abbreviations: M, methionine; g, guide RNA; C, conserved cysteine; B, conserved basic amino acids; CH, Conserved cysteine and histidine; ZDD, zinc-binding dimerization domain; NBD, nonamer binding domain; DDBD, dimerization and DNA-binding domain; pre-R, pre-RNase H; RNH, catalytic RNase H; CTD, carboxy-terminal domain.

FIG. 2. Guide RNA screening for RAG1 exonic strategies

(A) Schematic representation of gene editing experiment performed in NALM6-WT cells edited by six gRNAs targeting RAG1 exon 2, guide9 (g9, targeting the intronic region) as negative control, and guide 14 (g14, targeting the Methionine downstream Methionine 5 causing gene disruption) as positive control. (B) Graph shows frequency of cutting efficiency of the first six gRNAs assessed ten days upon gRNA delivery by a T7 mismatch selective endonuclease assay. (C) Analysis of RAG1 protein expression and housekeeping protein p38 as control by Western blot assay. (D) Graph shows frequency of GFP+ cells as surrogate of RAG1 recombination activity in bulk NALM6-WT edited cells and in NALM6 cell line lacking RAG1 gene (NALM6.Rag1-KO clone) assessed 7 days after serum-starvation by flow cytometry. (E) Graph shows frequency of insertion and deletion (indel) obtained from single edited clones by TIDE analysis of Sanger sequences. (F) Graph shows frequency of GFP+ cells as surrogate of RAG1 recombination activity in selected mono- and bi-allelic edited clones assessed 7 days after serum-starvation by flow cytometry.

FIG. 3. Analysis of cutting efficiency of exonic GRNAs in cd34+ cells from mobilized peripheral blood

(A) Schematic representation of gene editing protocol performed to deliver gRNA in CD34+ cells derived from mobilized peripheral blood (MPB-CD34+) of a healthy donor (HD). (B) Graph shows frequency of cutting efficiency of the first six guides assessed ten days upon gRNA delivery by a T7 mismatch selective endonuclease assay.

FIG. 4. Analysis of cutting efficiency of gRNAs designed for replacement strategies

(A) Graph shows frequency of cutting efficiency of the first six gRNAs targeting RAG1 exon 2, and g9 as control, assessed ten days upon gRNA delivery by a T7 mismatch selective endonuclease assay. (B) Schematic representation of the “Intron 1 RAG1 gene replacement strategy”. Abbreviations: HA, homology arm; SA, splice acceptor; coRAG1 CDS, codon optimized RAG1 coding sequence; BGHpA, bovine growth hormone poly A; Ex., exon; gRNA, guide RNA; 3′UTR, 3′ untranslated region; HDR, homology directed repair.

FIG. 5. Corrective donor templates

(A) Schematic representation of corrective donor templates specific for “g5 M3 ex2 RAG1” (g5) and “g6 M2 ex2 RAG1” (g6) gRNAs. One donor for the gene targeting strategy and two donors for the replacement strategy have been shown for g5 and g6. (B) Schematic representation of donor templates specific for the gene replacement strategy exploiting the following gRNAs: “g7 exon2 M2/3” (g7), “g10 exon2 M2/3” (g10), “g13 exon2 M2/3” (g13), “g8 exon2 M2/3” (g8), “g9 exon2 M2/3” (g9), “g12 exon2 M2/3” (g12), “g11 exon2 M2/3” (g11) or “g14 exon2 M5” (g14). (C) Schematic representation of the corrective donor suitable for the “intron 1 RAG1 gene replacement” strategy. (A-C) Abbreviations: 5′ and 3′ ITR, inverted terminal repeat; L-HA, left homology arm; SA, splice acceptor; c.o., codon optimized; R-HA, right homology arm.

FIG. 6. Generation of NALM6 Cas9 and K562 Cas9 cell lines

A) Schematic representation of the protocol for generation of K562 Cas9 and NALM6 Cas9 cell lines; B) Vector Copy Number (VCN) of the integrated Cas9 containing cassette measured by ddPCR, telomerase was used as normalizer; C) Cas9 expression for scaling doses of doxycycline measured by qPCR in NALM6 Cas9 (left panel) and K562 Cas9 (right panel) cell lines, represented as fold change Vs actin.

FIG. 7. Selection of the best performing gRNA

A) Schematic representation of the intronic and exonic loci targeted by the different gRNA tested; B) Schematic representation of the experimental protocol; C) Percentages of NHEJ induced indels in K562 Cas9 treated with different doses of plasmids encoding for different guides, 7 days after transfection, n=1; D) Percentages of NHEJ induced indels in NALM6 Cas9 treated with different doses of plasmids encoding for guides 3, 7 and 9, 7 days after transfection, n=1; E) Percentages of NHEJ induced indels in NALM6 Cas9 treated with different doses of guides 3 and 9 in vitro preassembled RNPs 7 days after transfection, n=1.

FIG. 8. Off-target analysis

A) Table shows the top 10 off-target sites predicted by in silico COSMID tool for guide 9. The off-target sequence, type of PAM, score, number of mismatches and chromosomal position are shown. B-C) Cutting efficiency measured as percentage of NHEJ (D) and dsDNA tag integration (ODN) on target site are evaluated by RFLP in K562 cells. D-E) Plots show the coverage of on-target reads (chromosome 11) of guide 9 (D) and guide 7 (E) and off-target reads identified for guide 7 by relaxed constraints (chromosome 20 and 9). F) Percentages of NHEJ induced indels in hCB-CD34+ cells treated with different doses of guides 3 and 9 as in vitro preassembled RNPs, n=2;

FIG. 9. Exonic gene editing strategy exploiting g6/AAV6 donor sets on NALM6.Rag1-KO cells

(A) Schematic representation of gene editing experiment performed in NALM6.Rag1-KO cells electroporated with gRNA 6 (g6)/Cas9 RNP and transduced with AAV6 donor for the exon 2 RAG1 gene targeting strategy or with AAV6 donor for the exon 2 RAG1 gene replacement strategy with long right homology arm (HAR). Bulk edited cells were subcloned and mono- and bi-allelic edited clones were selected by HDR analysis (ddPCR). (B) Graph shows the proportion of edited alleles in single clones performed by ddPCR. Clone 11 showed a bi-allellic editing. (C) Graph shows the transduction efficiency of LV-invGFP measured as proportion of CD4+ cells by flow cytometry seven days after serum starvation. (D) Recombination activity was evaluated 7 days after serum-starvation as proportion of GFP+ cells gated on transduced cells by flow cytometry. NALM6-WT cells and NALM6.Rag1-KO cells are used as positive and negative controls, respectively. (E) Graph summarizes the recombination activity of NALM6-WT cells as bulk or single clones, NALM6.Rag1-KO cells and bi- and mono-allelic edited clones evaluated 4 days after serum-starvation as proportion of GFP+ cells gated on transduced cells by flow cytometry. Mann-Whitney test; P values: *<0.05; **<0.005; ***<0.0005; ****<0.0001; Mean±SD are shown. (F) Exogenous c.o.RAG1 expression was measured in edited clones not starved or four days after starvation by RT-qPCR and shown as relative expression to beta-actin used as housekeeping gene. (G) Exogenous c.o.RAG1 expression levels, measured as described in panel F, are shown according to each experimental group. Wilcoxon matched-pairs signed rank test between not starved and starved samples; P values: *<0.05; **<0.005; ***<0.0005; ****<0.0001; Mean±SD are shown. (H) Endogenous c.o.RAG1 expression was measured in NALM6-WT bulk cells and in NALM6-WT single clones not starved or four days after starvation by RT-qPCR and shown as relative expression to beta-actin used as housekeeping gene. Wilcoxon matched-pairs signed rank test between not starved and starved samples; P values: *<0.05; **<0.005; ***<0.0005; ****<0.0001; Mean±SD are shown.

FIG. 10. Exonic gene editing strategy exploiting g6/AAV6 donor sets on Human HSPC

(A) Schematic representation of gene editing experiment performed in human CD34+ cells isolated from mobilized peripheral blood (mPB) of two healthy donors (HDs). Cells were electroporated with gRNA 6 (g6)/Cas9 RNP and transduced with AAV6 donor for the exon 2 RAG1 gene targeting strategy or with AAV6 donor for the exon 2 RAG1 gene replacement strategy which carries the long right homology arm (HAR). (B) Proportion of edited alleles analyzed by ddPCR on bulk untreated and edited CD34⁺ cells 4 days after the editing. (C) Graph shows cell growth curves of untreated (UT) and edited cells with targeting (Target. AAV6) or replacement (Replac. AAV6) after the editing procedure. (D) Distribution of the CD34⁺ cell subpopulations and CD34⁻ cells measured by flow cytometry based on the expression of hCD133 and hCD90 analysed 4 days after the editing. (E) Representative plots of the T cell differentiation stages analysed by flow cytometry 7 weeks after ATO seeding with CD34⁺ cells untreated (UT) or edited by g6 gRNA with the targeting AAV6 donor (TARGET. AAV6) or the replacement AAV6 donor (REPLAC. AAV6). (F) Kinetics of TCRαβ⁺ CD3⁺ cells analyzed by flow cytometry over time upon ATO seeding.

FIG. 11. Screening and selection of gRNAs for RAG1 exonic strategies

(A) Schematic representations of RAG1 gene, protein domains and gRNA positions mapping at the 5′ region of RAG1 exon 2. (B) Schematic representation of gene editing experiment performed in NALM6-WT cells edited by eight gRNAs targeting RAG1 exon 2. gRNA 14 (g14×KO) targeting the Methionine downstream Methionine 5 represents as positive control of RAG1 gene disruption. (C) Graph shows frequency of cutting efficiency of various gRNAs assessed seven days upon gRNA delivery by a T7 mismatch selective endonuclease assay. (D) Graph shows frequency of GFP+ cells as surrogate of RAG1 recombination activity in bulk NALM6-WT edited cells and in NALM6 cell line lacking RAG1 gene (NALM6.Rag1-KO clone) assessed 7 days after serum-starvation by flow cytometry.

FIG. 12. Analysis of cutting and RAG1-disruption efficiency of exonic gRNAs in CD34⁺ cells from mobilized peripheral blood

(A) Schematic representation of gene editing protocol performed to deliver nine gRNAs in CD34⁺ cells derived from mobilized peripheral blood (mPB-CD34⁺) of a healthy donors (HDs). gRNA 14 (g14×KO) targeting the Methionine downstream Methionine 5 represents as positive control of RAG1 gene disruption. gRNA 9 (g9) targeting the intronic region represent the negative control. (B) Graph shows frequency of cutting efficiency of gRNAs assessed 7 days upon gRNA delivery by a T7 mismatch selective endonuclease assay (HD_A and B are shown). (C) Representative plots of the T cell differentiation stages analysed by flow cytometry 6 weeks after ATO seeding and editing of CD34⁺ cells with gRNAs (HD_A is shown). (D) Proportion of TCRαβ+CD3⁺ cells were analyzed by flow cytometry 6 weeks upon ATO seeding and shown the levels of RAG1 disruption in terms of TCR recombination (HD_A and B are shown). (E) Kinetics of TCRαβ+CD3⁺ cells analyzed by flow cytometry over time upon ATO seeding (HD_A is shown). (F) Graph shows frequency of cutting efficiency of gRNAs in ATO-derived T cells 7 weeks upon ATO seeding assessed by a T7 mismatch selective endonuclease assay (HD_A (light grey circle) and HD_B (dark grey circle) are shown).

FIG. 13. Additional corrective donor templates

(A) Schematic representation of corrective donor templates specific for “g6 M2 ex2 RAG1” (g6), “g11 exon2 M2/3” (g11), and “g13 exon2 M2/3” (g13) gRNAs. Donors for the gene targeting and the replacement strategies have been shown for g6, g11, and g13. An additional donor template has been designed for the replacement strategy exploiting g6 with a short right homology arm (shown in the first lane of g6 donors). Abbreviations: 5′ and 3′ ITR, inverted terminal repeat; L-HA, left homology arm; SA, splice acceptor; c.o., codon optimized; R-HA, right homology arm.

FIG. 14. Exonic gene editing strategy exploiting g11/AAV6 and g13/AAV6 donor sets on NALM6.Rag1-KO cells

(A) Schematic representation of gene editing experiment performed in NALM6.Rag1-KO cells electroporated with g11/Cas9 RNP or g13/Cas9 RNP and transduced with AAV6 donor for the targeting strategy or for the replacement strategy. (B) Proportion of edited alleles was analyzed by ddPCR on bulk untreated and edited NALM6.Rag1-KO cells 4 days after the editing. (C) Graph shows frequency of GFP+ cells measured by flow cytometry as surrogate of RAG1 recombination activity in bulk NALM6-WT (WT) edited cells, in NALM6 cell line lacking RAG1 gene (KO) and in edited NALM6.Rag1 KO cells assessed 4 and 7 days after starvation induced by CDK4/6 inhibitor (CDK4/6i) or serum deprivation (no FBS).

FIG. 15. Exonic gene editing strategy exploiting g11/AAV6 and g13/AAV6 donor sets on human HSPC

(A) Schematic representation of gene editing experiment performed in human CD34⁺ cells isolated from mobilized peripheral blood (mPB) of two healthy donors (HDs). Cells were electroporated with gRNA/Cas9 RNP and transduced with AAV6 donor for the targeting or the donor strategy. (B) Proportion of edited alleles was analyzed by ddPCR on bulk untreated and edited CD34⁺ cells four days after the editing. (C) Editing efficiency on bulk HSPC is shown in terms of HDR, analyzed by ddPCR, and NHEJ, analyzed by T7 mismatch selective endonuclease assay, four days upon gene editing. (D) Distribution of the CD34⁺ cell subpopulations and CD34⁻ cells measured by flow cytometry based on the expression of hCD133 and hCD90 analysed four days after gene editing. (E) Colony forming unit (CFU) assay was performed on untreated or edited HSPC by counting the number of red (erythroid), white (myeloid) and mixed colonies at microscope 14 days after the plating.

FIG. 16. Exonic gene editing strategy exploiting g11-g13/AAV6 donor sets on NALM6.Rag1-KO cells

(A) Schematic representation of gene editing experiment performed in NALM6.Rag1-KO cells electroporated with sgRNA 11 or 13 (g11 or g13)/Cas9 RNP and transduced with AAV6 donor for the exon 2 RAG1 gene targeting strategy or with AAV6 donor for the exon 2 RAG1 gene replacement strategy with long right homology arm (HAR). Bulk edited cells were subcloned and mono- and bi-allelic edited clones were selected by HDR analysis (ddPCR). (B) Recombination activity was evaluated 7 days after serum-starvation induced by CDK4/6 inhibitor as proportion of GFP+ cells gated on transduced cells by flow cytometry. NALM6-WT (WT) cells and NALM6.Rag1-KO (KO) cells were used as positive and negative controls, respectively. Bi-allelic edited clone (clone 69 edited by g11 and targeting donor) was indicated by the asterisk. (C) Exogenous codon optimized RAG1 expression was measured in edited clones not starved or four days after starvation by RT-qPCR and shown as relative expression to beta-actin used as housekeeping gene. Wilcoxon matched-pairs signed rank test between not starved and starved samples; P values: *<0.05; **<0.005; ***<0.0005; ****<0.0001; Mean±SD are shown.

FIG. 17. Editing and correction efficiency of exonic gene editing strategy exploiting g11-g13/AAV6 donor sets in human HSPCs

(A) Schematic representation of gene editing experiment performed in human CD34⁺ cells isolated from mobilized peripheral blood (MPB) of healthy donors (HDs) and RAG1-patient (RAG1-PT). Cells were electroporated with sgRNA 11 or 13 (g11 or g13)/Cas9 RNPs and transduced with AAV6 targeting or replacement donor in presence of HDR enhancers. (B) Proportion of edited alleles analyzed by ddPCR on bulk untreated and edited CD34⁺ cells 4 days after the editing. Graph shows cumulative data of two independent experiments. (C) Distribution of the CD34⁺ cell subpopulations and CD34⁻ cells measured by flow cytometry based on the expression of hCD133 and hCD90 analysed 4 days after the editing. (D) Representative plots of the T cell differentiation stages analysed by flow cytometry 6.5 weeks after ATO seeding. (E) Kinetics of TCRαβ+CD3⁺ cells analyzed by flow cytometry over time upon ATO seeding with untreated (UT) or edited HD (top panel) and RAG1-patient cells (bottom panel). (F-G) Simpson complexity index measuring the clonal diversity of TRB repertoire (F) and frequency of top 10 productive rearrangements (G) were analyzed by ImmunoSEQ assay in ATO-derived TCRαβ+CD3⁺ cells sorted 6.5 weeks post-seeding and in bulk cells isolated from ATO 7.5 weeks post-seeding.

FIG. 18. In Vivo transplantation of edited hMPB-CD34+ cells from HD and RAG1-Patient

(A) Kinetics of human cell engraftment measured by flow cytometry as frequency of hCD45⁺ cells in peripheral blood (PB) of NSG mice transplanted with untreated (UT) and edited hMPB-HSPCs derived from healthy donor (HD) and RAG1-patient (Pt). (B) Kinetics of HDR efficiency in PB tested over time after the transplant (Tx) by ddPCR. (C) Immune cell distribution in PB of transplanted mice measured by flow cytometry according to the expression of hCD19 (B cells), hCD3 (T cells) and hCD13 (myeloid cells) in the hCD45⁺ gate. (D) Distribution of hematopoietic populations in bone marrow 18 weeks after the transplant measured by flow cytometry. (E) Relative frequencies of stages of B cell differentiation were analyzed by flow cytometry in bone marrow cells according to the expression of hCD45, hCD45, hCD34, hCD19, hCD22, hCD10 and hCD20. (F) Molecular analysis of HDR on bone marrow cells analyzed by ddPCR. (G) Proportion of TCRαβ⁺ CD3⁺ cells in thymus of transplanted mice analyzed 18 weeks after the transplant by flow cytometry. (H) Molecular analysis of HDR on thymocytes analyzed by ddPCR.

FIG. 19. Off-target analysis for g11 and g13

(A) Schematic representations of editing of K562 cells co-electroporated with the sgRNA of interest (g11 or g13) and the double strand oligodeoxynucleotide (dsODN) to tag off-target integrations. Cutting efficiency, Tag integration and Guide-Seq analyses were performed 10 days upon electroporation. (B-C) Cutting efficiency measured as percentage of NHEJ (B) and dsODN tag integration (ODN) (C) on the on-target sites were evaluated by RFLP in K562 cells. (D) Summary table showing the total number of off-target sites (OT) identified for g11 and g13 sgRNAs.

### DETAILED DESCRIPTION

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

All recited genomic locations are based on human genome assembly GRCh38.p13 (GCF_000001405.39). One of skill in the art will be able to identify the corresponding genome locations in alternative genome assemblies and convert the recited genomic location accordingly. For example, RAG1 is located at chr 11:36510353 to 36579762 in assembly GRCh38.p13 and at chr 11:36532053 to 36601312 in assembly GRCh37.p13.

The publications discussed herein are provided solely for their disclosure prior to the filing date of the present application. Nothing herein is to be construed as an admission that such publications constitute prior art to the claims appended hereto.

### Recombination Activating Gene 1 (RAG1)

The present invention relates to methods for gene-editing cells to introduce a RAG1 polypeptide or a RAG1 polypeptide fragment, for example as a treatment for severe combined immunodeficiency. The present invention also relates to polynucleotides, vectors, guide RNAs, kits, compositions, and gene editing systems for use in said methods, and genomes and cells obtained or obtainable by said methods.

### The RAG1 Gene

“RAG1” is the abbreviated name of the polypeptide encoded by recombination activating gene 1 and is also known as RAG-1, RNF74, and recombination activating 1.

RAG1 is the catalytic component of the RAG complex, a multiprotein complex that mediates the DNA cleavage phase during V(D)J recombination. V(D)J recombination assembles a diverse repertoire of immunoglobulin and T-cell receptor genes in developing B and T-lymphocytes through rearrangement of different V (variable), in some cases D (diversity), and J (joining) gene segments. In the RAG complex, RAG1 mediates the DNA-binding to the conserved recombination signal sequences (RSS) and catalyses the DNA cleavage activities by introducing a double-strand break between the RSS and the adjacent coding segment. RAG2 is not a catalytic component but is required for all known catalytic activities.

The gene encoding RAG1 (NCBI gene ID: 5896) is located in the human genome at chr 11:36510353 to 36579762.

Several alternative mRNAs are transcribed from the RAG1 gene. Transcript variant 1 (NM_000448) has two exons and one intron. As used herein, the region of the RAG1 gene corresponding to the first exon of transcript variant 1 is called the “RAG1 exon 1”, the region of the RAG1 gene corresponding to the intron of transcript variant 1 is called the “RAG1 intron 1”, and the region of the RAG1 gene corresponding to the second exon (which encodes a RAG1 polypeptide) is called the “RAG1 exon 2”.

Suitably, the RAG1 exon 1 is from chr 11:36568006 to chr 11:36568122; the RAG1 intron 1 is from chr 11:36568123 to chr 11:36573290; and/or the RAG1 exon 2 is from chr 11:36573291 to chr 11:36579762.

Suitably, the RAG1 exon 1 consists of the nucleotide sequence of SEQ ID NO: 1, or variants thereof; the RAG1 intron 1 consists of the nucleotide sequence of SEQ ID NO: 2, or variants thereof; and/or the RAG1 exon 2 consists of the nucleotide sequence of SEQ ID NO: 3, or variants thereof.

Illustrative RAG1 exon 1
(SEQ ID NO: 1)
agaaacaagagggcaaggagagagcagagaacacactttgccttctctttggtattgagtaatatcaaccaaattgc
agacatctcaacactttggccaggcagcctgctgagcaag
Illustrative RAG1 intron 1
(SEQ ID NO: 2)
gtaacactcatacttttcatgccttgagccaaaatatttattacatttttatgtttctaactagaagtgcttgagctttttttccttcc
aggtgatgaggggatggaatgagcaaagctacatcaatttttttttaatgtatgaaaataaaaaaggtacaagaggcc
aagtttagggccactgaaggttcatagaaagatgcaaaatatctgaattactataaatgaatgctattgtcagaggaaa
ggtttaaggagtgcttcttgaatgaatgtgtacaaatcagcagaaggtaaggtgtgagactcttggaaatgaatactggt
agttcaggtgagaaaaataatcaggaacataatagggtgggaggaaatgtatggtttcccaggtattaacaagtattg
ccaggcatttcctgaactagattggcctaagtaggagaccaatgtttctcaaaatattcactcattttagaatcactgaatg
tttaaaaatgcaatttctggattccttcccaaacagccagactctttgggacctgatgatctgcatttctttttaaaaacaaa
ctcgctcatgattctgatttgtattaattttgagaattgccatggtagagaccctgctttgaggttatgttcttgagtcaggattc
ctggccagggattgtgatgatatatttctctttctgaagtggttcatgcaagaggttgtctgaaggaagagcaagaattgt
agtgttattttgtggatacttgagacttataaaaaggctttttattttgtcacatttttgatacatgatgtttggcaaaaaacaga
cgatagtatttgcagagtgaatgaataagtggaacaggtgtgataatgagaggtcacacttgagcacacagttattact
tggaaattgtgtacagactaagttgaagatgttaggagggaagattgtgggccaagtaacggggtgtatgtgtgtgggt
atagggtgggcagctgggatggaaatggggggctgctgctgctgctgcaccctggcctcctgaactaatgatatcact
caccagaaactactgttcctgcactgtccaagccaccccaaactagtttgtcaaaatgaatctgtgctgtgtggaggga
ggcacgcctgtagctctgatgtcagatggcaatgtcgagatggcagtggccggtggggacagggctgagccagcac
caaccactcagcctttgagatcccgaggctggtctactgctgagaccttttgttagaagagaggagatcaagcatttgc
aaggtttctgagtgtcaaaatatgaatccaagataactctttcacaatcctaacttcatgctgtctacaggtccatattttag
cctgctttctccatgttcatccgaaaagaaagaaaagctaagggtggtggtcatatttgaaattagccagatcttaagtttt
tctgggggaaatttagaagaaaatatggaaaagtgactatgagcacatatacagctagtctttaaaacagttttatcca
aaataaatgtatcacaaaattaataaaaatagttacttgcttgttttgaataattcaaatgatacaaaaattaataaaataa
aaagtgcaaaaggccctcttatcaatgccaattctatttttttcagaaattaaacactgttaagattttagtgtgtatcctttca
gaattcctgtgatttcatatatgtacaaatacaaacgtatctacataaagggaatcctactatacttgctattgtcattctattc
tctgctttttcatgtgagcatctttccatgtcactgatgcatacagaaattgcacatatgcatcagtgcatacagaaaattaa
attttctgcatggttttccactgtatgtctggaccatagtttatttaataataatgccctttgggtaattatttatattgtttcctgcttt
ttcaaagtaacagcttttgaaacaaatctctctctgtctttatataaatattgttgcattcctgtggaaatgtttctattggataa
cttcccaaaaggagatttattgcatcaaagataatatattcaaaaattttaaagatattgctaaattgtctagtaggtatttta
taccaatttatactcctcccaagaatgtatggagatatcttaatttctccatgccttcattaatgctgaaccatataagtagttt
taatctttgctaattgaatagataaaaaatatctaatctaagtctagttcttaaaagttctatcttctaccaaaagtaatacac
gtctattttagggagtaaaaatcacaagtaaggataaaaaatagtgcagcaataaacacaggagtgtagatgtctctg
aacatactgatttaacttcctttggataaatacccagtagtaggactgctggatcatataataattctatctttagtttttttgag
gacctccatactattcttcatagtggctgtactaatttacattcctaccaactgtgtatgaaggttcccttttctctacatccttg
ccagcattcattattgcttgtcatttggatacaatctattttaactggggtgagatgacatctcattgtagttttgatatgcatttc
tctgatgatcagtggtgttgagcaccttttcatatacctgtttgccatttgtatgtcttcctttgagaaatgtctattcagatatttt
acctattttaaaatcggattattagattgtttcctgtagagttgtttgagctccttgtatattctggttattaatctcttgtcagatgc
atagcttacaaatattttctcccatcatgtggattgtgtcttcactttgtggattgtttactttgctgtgcagaagcttttaacttga
tgcaatcccatttgtccacttttgctttggttgccttccacaggagtatttaaataaatgtagtttggtagattttggtatagtaat
gcaggccagtgggagtcaggggagaaatgtgtagggaagtgagatagttctaaggatcctacaaacatgccttatga
ttgacttactcaatgtgaaagtcaatattaaacttgatgagctctagagatggtcatgcattttaaaaagaattactcaaaa
tattgtcttggaataccagagagcaagtgctttaagtataggctgggaagtaaaatgctaaaggaatgagaaggcattt
ggggttgagttcaacctaagaggcaggggagccacagggaaagacctagcacctgccacagaagagaattagg
aagcagaattgaactataagcaattttgaggtgttcgttgggctgcagttgaaatattttttgaggttaatgagacatttgaa
atggccgtgtattgtttaactcttgcatagtcctgcatagggaacaatctaataggatttctctgtgaatcaagtcttagaaa
tttgcttttaatttttatgaaaaacgcccatttctttgtttttgagacagagtcctgctctgtcatccaggctgggttgcagtggc
gtgatcttggcccactgcaatctctgcctcctgggttcaggcaattttcctgtctcagcctcccgagtagctgggatttcaa
gtgcctgccaccatgcccggctaaatttttttgtatttttggtacagatggagtatcaccatgttggccaggctggtctcgaa
ctcctgacctcaagtgattcaccagccttgacctcccaaagtgttgggatcacaggcatgagccactgtgcctgtgccc
caaaacaccaatttctgatgtgtgatgcatgtaagatagaacaaacttcagtaaagcggggacttgaaaagaggcttt
ggtaacagctgtcagcattaacccttgcccctccgtacctcctaatcccacccctgctcaaagtatgttcatctgagaattt
gtctccataactatgtgactataaaaattctcatcgattttgttagttgatcaattgagggaaaaacatatgttacttgatata
actggtgggtcaaaagaattaacccaggcaaatttgagataggtggatgggatgatggattgaaaatacagctgctct
ctttccaatcatgtactaagtaatttgggaaagattgatctaattgggtctagagagtacacttcacatggcattgtttgactt
tttttctgcatcgctagcgatctgtgcattacaactcaaatcagtcgggtttcctggcatatgtaattgccaatgttttttacca
gaagagaaacattactcccacctcttcttattatgttacaaactatagtgctaatgaccatcgaccaacagtgactttcag
gatgacctgtgtgagttttatctgaaaccatgtgaatttttcatcttaaaagtcccttagaatctcagtctatgtacactcaggt
ttgttgcaggtttagagttccgtgttttttgtttctaatgtagacacagccttataatttacaacagcattcactaattaaaattgt
aagcataattactatccacgatacttattattagtttgcattcataaagctcaaaattcacttcatcctttcaagtagtgaata
attagtttctttgggtttgcagctttatcatccttttatgacccatttggaagaaataaacaaccaaccccctggaagactgc
tttaaaaagctggaaatacattgtccagctagtacaatgaggctaatacaatgtggaaaatattacttttctttgattttagt
agcctgtttatctttacatttactgaacaaataactattgagcacctaatgtatactgggacccttggggaggcaaagatg
aatcaaagattctgtccttaaagaccttaaggtttttgtggaaggaaataaaactttacatgtatatatttaagcacttatat
gtgtgtaacaggtataagtaaccataaacactgtcagaagaggaaataactctatgatcagcacctaacatgatatatt
aaggtagaagatttaatacatatcttttggaatacatgaataaataattgaatgtatttatttttattatttataagatacatca
gtgggatattgatattggtcttaatatgacttgttttcattgttctcag
Illustrative RAG1 exon 2
(SEQ ID NO: 3)
gtacctcagccagcATGGCAGCCTCTTTCCCACCCACCTTGGGACTCAGTTCTGCCCC
AGATGAAATTCAGCACCCACATATTAAATTTTCAGAATGGAAATTTAAGCTGTTC
CGGGTGAGATCCTTTGAAAAGACACCTGAAGAAGCTCAAAAGGAAAAGAAGGAT
TCCTTTGAGGGGAAACCCTCTCTGGAGCAATCTCCAGCAGTCCTGGACAAGGC
TGATGGTCAGAAGCCAGTCCCAACTCAGCCATTGTTAAAAGCCCACCCTAAGTT
TTCAAAGAAATTTCACGACAACGAGAAAGCAAGAGGCAAAGCGATCCATCAAGC
CAACCTTCGACATCTCTGCCGCATCTGTGGGAATTCTTTTAGAGCTGATGAGCA
CAACAGGAGATATCCAGTCCATGGTCCTGTGGATGGTAAAACCCTAGGCCTTTT
ACGAAAGAAGGAAAAGAGAGCTACTTCCTGGCCGGACCTCATTGCCAAGGTTTT
CCGGATCGATGTGAAGGCAGATGTTGACTCGATCCACCCCACTGAGTTCTGCC
ATAACTGCTGGAGCATCATGCACAGGAAGTTTAGCAGTGCCCCATGTGAGGTTT
ACTTCCCGAGGAACGTGACCATGGAGTGGCACCCCCACACACCATCCTGTGAC
ATCTGCAACACTGCCCGTCGGGGACTCAAGAGGAAGAGTCTTCAGCCAAACTT
GCAGCTCAGCAAAAAACTCAAAACTGTGCTTGACCAAGCAAGACAAGCCCGTCA
GCACAAGAGAAGAGCTCAGGCAAGGATCAGCAGCAAGGATGTCATGAAGAAGA
TCGCCAACTGCAGTAAGATACATCTTAGTACCAAGCTCCTTGCAGTGGACTTCC
CAGAGCACTTTGTGAAATCCATCTCCTGCCAGATCTGTGAACACATTCTGGCTG
ACCCTGTGGAGACCAACTGTAAGCATGTCTTTTGCCGGGTCTGCATTCTCAGAT
GCCTCAAAGTCATGGGCAGCTATTGTCCCTCTTGCCGATATCCATGCTTCCCTA
CTGACCTGGAGAGTCCAGTGAAGTCCTTTCTGAGCGTCTTGAATTCCCTGATGG
TGAAATGTCCAGCAAAAGAGTGCAATGAGGAGGTCAGTTTGGAAAAATATAATC
ACCACATCTCAAGTCACAAGGAATCAAAAGAGATTTTTGTGCACATTAATAAAGG
GGGCCGGCCCCGCCAACATCTTCTGTCGCTGACTCGGAGAGCTCAGAAGCACC
GGCTGAGGGAGCTCAAGCTGCAAGTCAAAGCCTTTGCTGACAAAGAAGAAGGT
GGAGATGTGAAGTCCGTGTGCATGACCTTGTTCCTGCTGGCTCTGAGGGCGAG
GAATGAGCACAGGCAAGCTGATGAGCTGGAGGCCATCATGCAGGGAAAGGGCT
CTGGCCTGCAGCCAGCTGTTTGCTTGGCCATCCGTGTCAACACCTTCCTCAGCT
GCAGTCAGTACCACAAGATGTACAGGACTGTGAAAGCCATCACAGGGAGACAG
ATTTTTCAGCCTTTGCATGCCCTTCGGAATGCTGAGAAGGTACTTCTGCCAGGC
TACCACCACTTTGAGTGGCAGCCACCTCTGAAGAATGTGTCTTCCAGCACTGAT
GTTGGCATTATTGATGGGCTGTCTGGACTATCATCCTCTGTGGATGATTACCCA
GTGGACACCATTGCAAAGAGGTTCCGCTATGATTCAGCTTTGGTGTCTGCTTTG
ATGGACATGGAAGAAGACATCTTGGAAGGCATGAGATCCCAAGACCTTGATGAT
TACCTGAATGGCCCCTTCACTGTGGTGGTGAAGGAGTCTTGTGATGGAATGGG
AGACGTGAGTGAGAAGCATGGGAGTGGGCCTGTAGTTCCAGAAAAGGCAGTCC
GTTTTTCATTCACAATCATGAAAATTACTATTGCCCACAGCTCTCAGAATGTGAA
AGTATTTGAAGAAGCCAAACCTAACTCTGAACTGTGTTGCAAGCCATTGTGCCTT
ATGCTGGCAGATGAGTCTGACCACGAGACGCTGACTGCCATCCTGAGTCCTCT
CATTGCTGAGAGGGAGGCCATGAAGAGCAGTGAATTAATGCTTGAGCTGGGAG
GCATTCTCCGGACTTTCAAGTTCATCTTCAGGGGCACCGGCTATGATGAAAAAC
TTGTGCGGGAAGTGGAAGGCCTCGAGGCTTCTGGCTCAGTCTACATTTGTACTC
TTTGTGATGCCACCCGTCTGGAAGCCTCTCAAAATCTTGTCTTCCACTCTATAAC
CAGAAGCCATGCTGAGAACCTGGAACGTTATGAGGTCTGGCGTTCCAACCCTTA
CCATGAGTCTGTGGAAGAACTGCGGGATCGGGTGAAAGGGGTCTCAGCTAAAC
CTTTCATTGAGACAGTCCCTTCCATAGATGCACTCCACTGTGACATTGGCAATG
CAGCTGAGTTCTACAAGATCTTCCAGCTAGAGATAGGGGAAGTGTATAAGAATC
CCAATGCTTCCAAAGAGGAAAGGAAAAGGTGGCAGGCCACACTGGACAAGCAT
CTCCGGAAGAAGATGAACCTCAAACCAATCATGAGGATGAATGGCAACTTTGCC
AGGAAGCTCATGACCAAAGAGACTGTGGATGCAGTTTGTGAGTTAATTCCTTCC
GAGGAGAGGCACGAGGCTCTGAGGGAGCTGATGGATCTTTACCTGAAGATGAA
ACCAGTATGGCGATCATCATGCCCTGCTAAAGAGTGCCCAGAATCCCTCTGCCA
GTACAGTTTCAATTCACAGCGTTTTGCTGAGCTCCTTTCTACGAAGTTCAAGTAT
AGGTATGAGGGAAAAATCACCAATTATTTTCACAAAACCCTGGCCCATGTTCCTG
AAATTATTGAGAGGGATGGCTCCATTGGGGCATGGGCAAGTGAGGGAAATGAG
TCTGGTAACAAACTGTTTAGGCGCTTCCGGAAAATGAATGCCAGGCAGTCCAAA
TGCTATGAGATGGAAGATGTCCTGAAACACCACTGGTTGTACACCTCCAAATAC
CTCCAGAAGTTTATGAATGCTCATAATGCATTAAAAACCTCTGGGTTTACCATGA
ACCCTCAGGCAAGCTTAGGGGACCCATTAGGCATAGAGGACTCTCTGGAAAGC
CAAGATTCAATGGAATTTTAAgtagggcaaccacttatgagttggtttttgcaattgagtttccctctgggttg
cattgagggcttctcctagcaccctttactgctgtgtatggggcttcaccatccaagaggtggtaggttggagtaagatgc
tacagatgctctcaagtcaggaatagaaactgatgagctgattgcttgaggcttttagtgagttccgaaaagcaacagg
aaaaatcagttatctgaaagctcagtaactcagaacaggagtaactgcaggggaccagagatgagcaaagatctgt
gtgtgttggggagctgtcatgtaaatcaaagccaaggttgtcaaagaacagccagtgaggccaggaaagaaattggt
cttgtggttttcatttttttcccccttgattgattatattttgtattgagatatgataagtgccttctatttcatttttgaataattcttca
tttttataattttacatatcttggcttgctatataagattcaaaagagctttttaaatttttctaataatatcttacatttgtacagcatg
atgacctttacaaagtgctctcaatgcatttacccattcgttatataaatatgttacatcaggacaactttgagaaaatcagt
ccttttttatgtttaaattatgtatctattgtaaccttcagagtttaggaggtcatctgctgtcatggatttttcaataatgaatttag
aatacacctgttagctacagttagttattaaatcttctgataatatatgtttacttagctatcagaagccaagtatgattctttat
ttttactttttcatttcaagaaatttagagtttccaaatttagagcttctgcatacagtcttaaagccacagaggcttgtaaaa
atataggttagcttgatgtctaaaaatatatttcatgtcttactgaaacattttgccagactttctccaaatgaaacctgaatc
aatttttctaaatctaggtttcatagagtcctctcctctgcaatgtgttattctttctataatgatcagtttactttcagtggattcag
aattgtgtagcaggataaccttgtatttttccatccgctaagtttagatggagtccaaacgcagtacagcagaagagtta
acatttacacagtgctttttaccactgtggaatgttttcacactcatttttccttacaacaattctgaggagtaggtgttgttatta
tctccatttgatgggggtttaaatgatttgctcaaagtcatttaggggtaataaatacttggcttggaaatttaacacagtcct
tttgtctccaaagcccttcttctttccaccacaaattaatcactatgtttataaggtagtatcagaatttttttaggattcacaac
taatcactatagcacatgaccttgggattacatttttatggggcaggggtaagcaagtttttaaatcatttgtgtgctctggct
cttttgatagaagaaagcaacacaaaagctccaaagggccccctaaccctcttgtggctccagttatttggaaactatg
atctgcatccttaggaatctgggatttgccagttgctggcaatgtagagcaggcatggaattttatatgctagtgagtcata
atgatatgttagtgttaattagttttttcttcctttgattttattggccataattgctactcttcatacacagtatatcaaagagcttg
ataatttagttgtcaaaagtgcatcggcgacattatctttaattgtatgtatttggtgcttcttcagggattgaactcagtatcttt
cattaaaaaacacagcagttttccttgctttttatatgcagaatatcaaagtcatttctaatttagttgtcaaaaacatataca
tattttaacattagtttttttgaaaactcttggttttgtttttttggaaatgagtgggccactaagccacactttcccttcatcctgct
taatccttccagcatgtctctgcactaataaacagctaaattcacataatcatcctatttactgaagcatggtcatgctggtt
tatagattttttacccatttctactctttttctctattggtggcactgtaaatactttccagtattaaattatccttttctaacactgta
ggaactattttgaatgcatgtgactaagagcatgatttatagcacaacctttccaataatcccttaatcagatcacattttga
taaaccctgggaacatctggctgcaggaatttcaatatgtagaaacgctgcctatggttttttgcccttactgttgagactg
caatatcctagaccctagttttatactagagttttatttttagcaatgcctattgcaagtgcaattatatactccagggaaattc
accacactgaatcgagcatttgtgtgtgtatgtgtgaagtatatactgggacttcagaagtgcaatgtatttttctcctgtga
aacctgaatctacaagttttcctgccaagccactcaggtgcattgcagggaccagtgataatggctgatgaaaattgat
gattggtcagtgaggtcaaaaggagccttgggattaataaacatgcactgagaagcaagaggaggagaaaaagat
gtctttttcttccaggtgaactggaatttagttttgcctcagatttttttcccacaagatacagaagaagataaagatttttttgg
ttgagagtgtgggtcttgcattacatcaaacagagttcaaattccacacagataagaggcaggatatataagcgccag
tggtagttgggaggaataaaccattatttggatgcaggtggtttttgattgcaaatatgtgtgtgtcttcagtgattgtatgac
agatgatgtattcttttgatgttaaaagattttaagtaagagtagatacattgtacccattttacattttcttattttaactacagt
aatctacataaatatacctcagaaatcatttttggtgattattttttgttttgtagaattgcacttcagtttattttcttacaaataac
cttacattttgtttaatggcttccaagagccttttttttttttgtatttcagagaaaattcaggtaccaggatgcaatggatttattt
gattcaggggacctgtgtttccatgtcaaatgttttcaaataaaatgaaatatgagtttcaatactttttatattttaatatttcca
ttcattaatattatggttattgtcagcaattttatgtttgaatatttgaaataaaagtttaagatttgaaaa

In the illustrative RAG1 exon 2 (SEQ ID NO: 3), upper case letters indicate a nucleotide sequence which encodes a RAG1 polypeptide.

### RAG1 Polypeptides

Isolated polynucleotides according to the present invention may comprise a nucleotide sequence encoding a RAG1 polypeptide, or a fragment thereof.

The RAG1 polypeptide may be a human RAG1 polypeptide. Suitably, the RAG1 polypeptide may comprise or consist of a polypeptide sequence of UniProtKB accession P15918, or a variant thereof.

A “RAG1 polypeptide” is a polypeptide having RAG1 activity, for example a polypeptide which is able to form a RAG complex, mediate DNA-binding to the RSS, and introduce a double-strand break between the RSS and the adjacent coding segment. Suitably, a RAG1 polypeptide may have the same or similar activity to a wild-type RAG1, e.g. may have at least 40%, at least 50%, at least 60%, at least 70%, at least 80%, at least 90%, at least 95%, at least 100%, at least 110%, at least 120%, at least 130%, at least 140%, or at least 150% of the activity of a wild-type RAG1 polypeptide.

A “RAG1 polypeptide variant” may include an amino acid sequence or a nucleotide sequence which may be at least 50%, at least 55%, at least 65%, at least 70%, at least 75%, at least 80%, at least 85% or at least 90% identical, optionally at least 95% or at least 97% or at least 99% identical to a wild-type RAG1 polypeptide. RAG1 variants may have the same or similar activity to a wild-type RAG1 polypeptide, e.g. may have at least at least 40%, at least 50%, at least 60%, at least 70%, at least 80%, at least 90%, at least 95%, at least 100%, at least 110%, at least 120%, at least 130%, at least 140%, or at least 150% of the activity of a wild-type RAG1 polypeptide.

A person skilled in the art would be able to generate RAG1 variants having the same or similar activity to a wild-type RAG1 polypeptide based on the known structural and functional features of RAG1 and/or using conservative substitutions. The minimal regions of RAG1 required for catalysis have been identified. These regions are referred to as the core proteins. Core RAG1 consists of multiple structural domains, termed the nonamer binding domain (NBD; residues 389-464), the central domain (residues 528-760), and the C-terminal domain (residues 761-980) domains. Besides the ability to recognize the RSS nonamer and heptamer through the NBD and the central domain, respectively, core RAG1 contains the essential acidic active site residues (Arbuckle, J. L., et al., 2011. BMC biochemistry, 12(1), p.23). Suitably, a variant of RAG1 comprises a nonamer binding domain, a central domain, and/or a C-terminal domain.

In some embodiments of the invention, a RAG1 polypeptide comprises or consists of an amino acid sequence which is at least 70% identical to SEQ ID NO: 4. Suitably, a RAG1 polypeptide comprises or consists of an amino acid sequence which is at least 80%, at least 85%, at least 90%, at least 95%, at least 98% or at least 99% identical to SEQ ID NO: 4.

In some embodiments, a RAG1 polypeptide comprises or consists of SEQ ID NO: 4.

RAG1 polypeptide isoform 1, UniProtKB accession P15918
(SEQ ID NO: 4)
MAASFPPTLGLSSAPDEIQHPHIKFSEWKFKLFRVRSFEKTPEEAQKEKKDSFEGKP
SLEQSPAVLDKADGQKPVPTQPLLKAHPKFSKKFHDNEKARGKAIHQANLRHLCRI
CGNSFRADEHNRRYPVHGPVDGKTLGLLRKKEKRATSWPDLIAKVFRIDVKADVDS
IHPTEFCHNCWSIMHRKFSSAPCEVYFPRNVTMEWHPHTPSCDICNTARRGLKRKS
LQPNLQLSKKLKTVLDQARQARQHKRRAQARISSKDVMKKIANCSKIHLSTKLLAVD
FPEHFVKSISCQICEHILADPVETNCKHVFCRVCILRCLKVMGSYCPSCRYPCFPTDL
ESPVKSFLSVLNSLMVKCPAKECNEEVSLEKYNHHISSHKESKEIFVHINKGGRPRQ
HLLSLTRRAQKHRLRELKLQVKAFADKEEGGDVKSVCMTLFLLALRARNEHRQADE
LEAIMQGKGSGLQPAVCLAIRVNTFLSCSQYHKMYRTVKAITGRQIFQPLHALRNAE
KVLLPGYHHFEWQPPLKNVSSSTDVGIIDGLSGLSSSVDDYPVDTIAKRFRYDSALV
SALMDMEEDILEGMRSQDLDDYLNGPFTVVVKESCDGMGDVSEKHGSGPVVPEK
AVRFSFTIMKITIAHSSQNVKVFEEAKPNSELCCKPLCLMLADESDHETLTAILSPLIA
EREAMKSSELMLELGGILRTFKFIFRGTGYDEKLVREVEGLEASGSVYICTLCDATRL
EASQNLVFHSITRSHAENLERYEVWRSNPYHESVEELRDRVKGVSAKPFIETVPSID
ALHCDIGNAAEFYKIFQLEIGEVYKNPNASKEERKRWQATLDKHLRKKMNLKPIMRM
NGNFARKLMTKETVDAVCELIPSEERHEALRELMDLYLKMKPVWRSSCPAKECPES
LCQYSFNSQRFAELLSTKFKYRYEGKITNYFHKTLAHVPEIIERDGSIGAWASEGNE
SGNKLFRRFRKMNARQSKCYEMEDVLKHHWLYTSKYLQKFMNAHNALKTSGFTM
NPQASLGDPLGIEDSLESQDSMEF

In some embodiments of the invention, a RAG1 polypeptide comprises or consists of an amino acid sequence which is at least 70% identical to SEQ ID NO: 5. Suitably, a RAG1 polypeptide comprises or consists of an amino acid sequence which is at least 80%, at least 85%, at least 90%, at least 95%, at least 98% or at least 99% identical to SEQ ID NO: 5.

In some embodiments, a RAG1 polypeptide comprises or consists of SEQ ID NO: 5.

RAG1 polypeptide isoform 2, UniProtKB accession P15918
(SEQ ID NO: 5)
MAASFPPTLGLSSAPDEIQHPHIKFSEWKFKLFRVRSFEKTPEEAQKEKKDSFEGKP
SLEQSPAVLDKADGQKPVPTQPLLKAHPKFSKKFHDNEKARGKAIHQANLRHLCRI
CGNSFRADEHNRRYPVHGPVDGKTLGLLRKKEKRATSWPDLIAKVFRIDVKADVDS
IHPTEFCHNCWSIMHRKFSSAPCEVYFPRNVTMEWHPHTPSCDICNTARRGLKRKS
LQPNLQLSKKLKTVLDQARQARQHKRRAQARISSKDVMKKIANCSKIHLSTKLLAVD
FPEHFVKSISCQICEHILADPVETNCKHVFCRVCILRCLKVMGSYCPSCRYPCFPTDL
ESPVKSFLSVLNSLMVKCPAKECNEEVSLEKYNHHISSHKESKEIFVHINKGGRPRQ
HLLSLTRRAQKHRLRELKLQVKAFADKEEGGDVKSVCMTLFLLALRARNEHRQADE
LEAIMQGKGSGLQPAVCLAIRVNTFLSCSQYHKMYRTVKAITGRQIFQPLHALRNAE
KVLLPGYHHFEWQPPLKNVSSSTDVGIIDGLSGLSSSVDDYPVDTIAKRFRYDSALV
SALMDMEEDILEGMRSQDLDDYLNGPFTVVVKESCDGMGDVSEKHGSGPVVPEK
AVRFSFTIMKITIAHSSQNVKVFEEAKPNSELCCKPLCLMLADESDHETLTAILSPLIA
EREAMKSSELMLELGGILRTFKFIFRGTGYDEKLVREVEGLEASGSVYICTLCDATRL
EASQNLVFHSITRSHAENLERYEVWRSNPYHESVEELRDRVKGVSAKPFIETVPSID
ALHCDIGNAAEFYKIFQLEIGEVYKNPNASKEERKRWQATLDKHLRKKMNLKPIMRM
NGNFARKLMTKETVDAVCELIPSEERHEALRELMDLYLKMKPVWRSSCPAKECPES
LCQYSFNSQRFAELLSTKFKYRN

In some embodiments of the invention, a RAG1 polypeptide comprises or consists of an amino acid sequence which is at least 70% identical to SEQ ID NO: 6. Suitably, a RAG1 polypeptide comprises or consists of an amino acid sequence which is at least 80%, at least 85%, at least 90%, at least 95%, at least 98% or at least 99% identical to SEQ ID NO: 6.

In some embodiments, a RAG1 polypeptide comprises or consists of SEQ ID NO: 6.

Illustrative RAG1 polypeptide
(SEQ ID NO: 6)
MAASFPPTLGLSSAPDEIQHPHIKFSEWKFKLFRVRSFEKTPEEAQKEKKDSFEGKP
SLEQSPAVLDKADGQKPVPTQPLLKAHPKFSKKFHDNEKARGKAIHQANLRHLCRI
CGNSFRADEHNRRYPVHGPVDGKTLGLLRKKEKRATSWPDLIAKVFRIDVKADVDS
IHPTEFCHNCWSIMHRKFSSAPCEVYFPRNVTMEWHPHTPSCDICNTARRGLKRKS
LQPNLQLSKKLKTVLDQARQARQRKRRAQARISSKDVMKKIANCSKIHLSTKLLAVD
FPEHFVKSISCQICEHILADPVETNCKHVFCRVCILRCLKVMGSYCPSCRYPCFPTDL
ESPVKSFLSVLNSLMVKCPAKECNEEVSLEKYNHHISSHKESKEIFVHINKGGRPRQ
HLLSLTRRAQKHRLRELKLQVKAFADKEEGGDVKSVCMTLFLLALRARNEHRQADE
LEAIMQGKGSGLQPAVCLAIRVNTFLSCSQYHKMYRTVKAITGRQIFQPLHALRNAE
KVLLPGYHHFEWQPPLKNVSSSTDVGIIDGLSGLSSSVDDYPVDTIAKRFRYDSALV
SALMDMEEDILEGMRSQDLDDYLNGPFTVVVKESCDGMGDVSEKHGSGPVVPEK
AVRFSFTIMKITIAHSSQNVKVFEEAKPNSELCCKPLCLMLADESDHETLTAILSPLIA
EREAMKSSELMLELGGILRTFKFIFRGTGYDEKLVREVEGLEASGSVYICTLCDATRL
EASQNLVFHSITRSHAENLERYEVWRSNPYHESVEELRDRVKGVSAKPFIETVPSID
ALHCDIGNAAEFYKIFQLEIGEVYKNPNASKEERKRWQATLDKHLRKKMNLKPIMRM
NGNFARKLMTKETVDAVCELIPSEERHEALRELMDLYLKMKPVWRSSCPAKECPES
LCQYSFNSQRFAELLSTKFKYRYEGKITNYFHKTLAHVPEIIERDGSIGAWASEGNE
SGNKLFRRFRKMNARQSKCYEMEDVLKHHWLYTSKYLQKFMNAHNALKTSGFTM
NPQASLGDPLGIEDSLESQDSMEF

### RAG1 Polypeptide Fragments

Isolated polynucleotides according to the present invention may comprise a nucleotide sequence encoding a RAG1 polypeptide fragment.

A “RAG1 polypeptide fragment” may refer to a portion or region of a full-length RAG1 polypeptide or variant thereof. Suitably, a RAG1 polypeptide fragment may be at least 50 amino acids in length, at least 100 amino acids in length, at least 150 amino acids in length, at least 200 amino acids in length, at least 250 amino acids in length, at least 300 amino acids in length, at least 350 amino acids in length, at least 400 amino acids in length, at least 450 amino acids in length, at least 500 amino acids in length, at least 550 amino acids in length, at least 600 amino acids in length, at least 650 amino acids in length, at least 700 amino acids in length, at least 750 amino acids in length, at least 800 amino acids in length, at least 850 amino acids, or at least 900 amino acids in length.

Suitably, the RAG1 polypeptide fragment may comprise at least the final 50 amino acids, at least the final 100 amino acids, at least the final 150 amino acids, at least the final 200 amino acids, at least the final 250 amino acids, at least the final 300 amino acids, at least the final 350 amino acids, at least the final 400 amino acids, at least the final 450 amino acids, at least the final 500 amino acids, at least the final 550 amino acids, at least the final 600 amino acids, at least the final 650 amino acids, at least the final 700 amino acids, at least the final 750 amino acids, at least the final 800 amino acids, at least the final 850 amino acids, or at least the final 900 amino acids of a full-length RAG1 polypeptide or variant thereof, optionally wherein 1 to 20 amino acids (e.g. about 15 amino acids) are absent from the C-terminus of the full-length RAG1 polypeptide or variant thereof.

In some embodiments of the invention, the RAG1 polypeptide fragment comprises or consists of an amino acid sequence which is at least 70% identical to any of SEQ ID NOs: 7-14 or 164-165. Suitably, the RAG1 polypeptide fragment comprises or consists of an amino acid sequence which is at least 80%, at least 85%, at least 90%, at least 95%, at least 98% or at least 99% identical to any of SEQ ID NOs: 7-14 or 164-165.

In some embodiments, the RAG1 polypeptide fragment comprises or consists of any of SEQ ID NOs: 7-14 or 164-165.

Illustrative RAG1 polypeptide fragment 1
(SEQ ID NO: 7)
SKIHLSTKLLAVDFPEHFVKSISCQICEHILADPVETNCKHVFCRVCILRCLKVMGSYC
PSCRYPCFPTDLESPVKSFLSVLNSLMVKCPAKECNEEVSLEKYNHHISSHKESKEI
FVHINKGGRPRQHLLSLTRRAQKHRLRELKLQVKAFADKEEGGDVKSVCMTLFLLA
LRARNEHRQADELEAIMQGKGSGLQPAVCLAIRVNTFLSCSQYHKMYRTVKAITGR
QIFQPLHALRNAEKVLLPGYHHFEWQPPLKNVSSSTDVGIIDGLSGLSSSVDDYPVD
TIAKRFRYDSALVSALMDMEEDILEGMRSQDLDDYLNGPFTVVVKESCDGMGDVSE
KHGSGPVVPEKAVRFSFTIMKITIAHSSQNVKVFEEAKPNSELCCKPLCLMLADESD
HETLTAILSPLIAEREAMKSSELMLELGGILRTFKFIFRGTGYDEKLVREVEGLEASGS
VYICTLCDATRLEASQNLVFHSITRSHAENLERYEVWRSNPYHESVEELRDRVKGV
SAKPFIETVPSIDALHCDIGNAAEFYKIFQLEIGEVYKNPNASKEERKRWQATLDKHL
RKKMNLKPIMRMNGNFARKLMTKETVDAVCELIPSEERHEALRELMDLYLKMKPVW
RSSCPAKECPESLCQYSFNSQRFAELLSTKFKYRYEGKITNYFHKTLAHVPEIIERD
GSIGAWASEGNESGNKLFRRFRKMNARQSKCYEMEDVLKHHWLYTSKYLQKFMN
AHNALKTSGFTMNPQASLGDPLGIEDSLESQDSMEF
Illustrative RAG1 polypeptide fragment 2
(SEQ ID NO: 8)
SKIHLSTKLLAVDFPEHFVKSISCQICEHILADPVETNCKHVFCRVCILRCLKVMGSYC
PSCRYPCFPTDLESPVKSFLSVLNSLMVKCPAKECNEEVSLEKYNHHISSHKESKEI
FVHINKGGRPRQHLLSLTRRAQKHRLRELKLQVKAFADKEEGGDVKSVCMTLFLLA
LRARNEHRQADELEAIMQGKGSGLQPAVCLAIRVNTFLSCSQYHKMYRTVKAITGR
QIFQPLHALRNAEKVLLPGYHHFEWQPPLKNVSSSTDVGIIDGLSGLSSSVDDYPVD
TIAKRFRYDSALVSALMDMEEDILEGMRSQDLDDYLNGPFTVVVKESCDGMGDVSE
KHGSGPVVPEKAVRFSFTIMKITIAHSSQNVKVFEEAKPNSELCCKPLCLMLADESD
HETLTAILSPLIAEREAMKSSELMLELGGILRTFKFIFRGTGYDEKLVREVEGLEASGS
VYICTLCDATRLEASQNLVFHSITRSHAENLERYEVWRSNPYHESVEELRDRVKGV
SAKPFIETVPSIDALHCDIGNAAEFYKIFQLEIGEVYKNPNASKEERKRWQATLDKHL
RKKMNLKPIMRMNGNFARKLMTKETVDAVCELIPSEERHEALRELMDLYLKMKPVW
RSSCPAKECPESLCQYSFNSQRFAELLSTKFKYRYEGKITNYFHKTLAHVPEIIERD
GSIGAWASEGNESGNKLFRRFRKMNARQSKCYEMEDVLKHHWLYTSKYLQKFMN
AHNALKTSGFTMNPQASLGDP
Illustrative RAG1 polypeptide fragment 3
(SEQ ID NO: 9)
EWHPHTPSCDICNTARRGLKRKSLQPNLQLSKKLKTVLDQARQARQRKRRAQARIS
SKDVMKKIANCSKIHLSTKLLAVDFPEHFVKSISCQICEHILADPVETNCKHVFCRVCI
LRCLKVMGSYCPSCRYPCFPTDLESPVKSFLSVLNSLMVKCPAKECNEEVSLEKYN
HHISSHKESKEIFVHINKGGRPRQHLLSLTRRAQKHRLRELKLQVKAFADKEEGGDV
KSVCMTLFLLALRARNEHRQADELEAIMQGKGSGLQPAVCLAIRVNTFLSCSQYHK
MYRTVKAITGRQIFQPLHALRNAEKVLLPGYHHFEWQPPLKNVSSSTDVGIIDGLSG
LSSSVDDYPVDTIAKRFRYDSALVSALMDMEEDILEGMRSQDLDDYLNGPFTVVVK
ESCDGMGDVSEKHGSGPVVPEKAVRFSFTIMKITIAHSSQNVKVFEEAKPNSELCC
KPLCLMLADESDHETLTAILSPLIAEREAMKSSELMLELGGILRTFKFIFRGTGYDEKL
VREVEGLEASGSVYICTLCDATRLEASQNLVFHSITRSHAENLERYEVWRSNPYHE
SVEELRDRVKGVSAKPFIETVPSIDALHCDIGNAAEFYKIFQLEIGEVYKNPNASKEE
RKRWQATLDKHLRKKMNLKPIMRMNGNFARKLMTKETVDAVCELIPSEERHEALRE
LMDLYLKMKPVWRSSCPAKECPESLCQYSFNSQRFAELLSTKFKYRYEGKITNYFH
KTLAHVPEIIERDGSIGAWASEGNESGNKLFRRFRKMNARQSKCYEMEDVLKHHW
LYTSKYLQKFMNAHNALKTSGFTMNPQASLGDPLGIEDSLESQDSMEF
Illustrative RAG1 polypeptide fragment 4
(SEQ ID NO: 10)
EWHPHTPSCDICNTARRGLKRKSLQPNLQLSKKLKTVLDQARQARQRKRRAQARIS
SKDVMKKIANCSKIHLSTKLLAVDFPEHFVKSISCQICEHILADPVETNCKHVFCRVCI
LRCLKVMGSYCPSCRYPCFPTDLESPVKSFLSVLNSLMVKCPAKECNEEVSLEKYN
HHISSHKESKEIFVHINKGGRPRQHLLSLTRRAQKHRLRELKLQVKAFADKEEGGDV
KSVCMTLFLLALRARNEHRQADELEAIMQGKGSGLQPAVCLAIRVNTFLSCSQYHK
MYRTVKAITGRQIFQPLHALRNAEKVLLPGYHHFEWQPPLKNVSSSTDVGIIDGLSG
LSSSVDDYPVDTIAKRFRYDSALVSALMDMEEDILEGMRSQDLDDYLNGPFTVVVK
ESCDGMGDVSEKHGSGPVVPEKAVRFSFTIMKITIAHSSQNVKVFEEAKPNSELCC
KPLCLMLADESDHETLTAILSPLIAEREAMKSSELMLELGGILRTFKFIFRGTGYDEKL
VREVEGLEASGSVYICTLCDATRLEASQNLVFHSITRSHAENLERYEVWRSNPYHE
SVEELRDRVKGVSAKPFIETVPSIDALHCDIGNAAEFYKIFQLEIGEVYKNPNASKEE
RKRWQATLDKHLRKKMNLKPIMRMNGNFARKLMTKETVDAVCELIPSEERHEALRE
LMDLYLKMKPVWRSSCPAKECPESLCQYSFNSQRFAELLSTKFKYRYEGKITNYFH
KTLAHVPEIIERDGSIGAWASEGNESGNKLFRRFRKMNARQSKCYEMEDVLKHHW
LYTSKYLQKFMNAHNALKTSGFTMNPQASLGDP
Illustrative RAG1 polypeptide fragment 5
(SEQ ID NO: 11)
EVYFPRNVTMEWHPHTPSCDICNTARRGLKRKSLQPNLQLSKKLKTVLDQARQAR
QRKRRAQARISSKDVMKKIANCSKIHLSTKLLAVDFPEHFVKSISCQICEHILADPVET
NCKHVFCRVCILRCLKVMGSYCPSCRYPCFPTDLESPVKSFLSVLNSLMVKCPAKE
CNEEVSLEKYNHHISSHKESKEIFVHINKGGRPRQHLLSLTRRAQKHRLRELKLQVK
AFADKEEGGDVKSVCMTLFLLALRARNEHRQADELEAIMQGKGSGLQPAVCLAIRV
NTFLSCSQYHKMYRTVKAITGRQIFQPLHALRNAEKVLLPGYHHFEWQPPLKNVSS
STDVGIIDGLSGLSSSVDDYPVDTIAKRFRYDSALVSALMDMEEDILEGMRSQDLDD
YLNGPFTVVVKESCDGMGDVSEKHGSGPVVPEKAVRFSFTIMKITIAHSSQNVKVF
EEAKPNSELCCKPLCLMLADESDHETLTAILSPLIAEREAMKSSELMLELGGILRTFK
FIFRGTGYDEKLVREVEGLEASGSVYICTLCDATRLEASQNLVFHSITRSHAENLERY
EVWRSNPYHESVEELRDRVKGVSAKPFIETVPSIDALHCDIGNAAEFYKIFQLEIGEV
YKNPNASKEERKRWQATLDKHLRKKMNLKPIMRMNGNFARKLMTKETVDAVCELIP
SEERHEALRELMDLYLKMKPVWRSSCPAKECPESLCQYSFNSQRFAELLSTKFKY
RYEGKITNYFHKTLAHVPEIIERDGSIGAWASEGNESGNKLFRRFRKMNARQSKCY
EMEDVLKHHWLYTSKYLQKFMNAHNALKTSGFTMNPQASLGDP
Illustrative RAG1 polypeptide fragment 6
(SEQ ID NO: 12)
RRGLKRKSLQPNLQLSKKLKTVLDQARQARQRKRRAQARISSKDVMKKIANCSKIH
LSTKLLAVDFPEHFVKSISCQICEHILADPVETNCKHVFCRVCILRCLKVMGSYCPSC
RYPCFPTDLESPVKSFLSVLNSLMVKCPAKECNEEVSLEKYNHHISSHKESKEIFVHI
NKGGRPRQHLLSLTRRAQKHRLRELKLQVKAFADKEEGGDVKSVCMTLFLLALRAR
NEHRQADELEAIMQGKGSGLQPAVCLAIRVNTFLSCSQYHKMYRTVKAITGRQIFQ
PLHALRNAEKVLLPGYHHFEWQPPLKNVSSSTDVGIIDGLSGLSSSVDDYPVDTIAK
RFRYDSALVSALMDMEEDILEGMRSQDLDDYLNGPFTVVVKESCDGMGDVSEKHG
SGPVVPEKAVRFSFTIMKITIAHSSQNVKVFEEAKPNSELCCKPLCLMLADESDHETL
TAILSPLIAEREAMKSSELMLELGGILRTFKFIFRGTGYDEKLVREVEGLEASGSVYIC
TLCDATRLEASQNLVFHSITRSHAENLERYEVWRSNPYHESVEELRDRVKGVSAKP
FIETVPSIDALHCDIGNAAEFYKIFQLEIGEVYKNPNASKEERKRWQATLDKHLRKKM
NLKPIMRMNGNFARKLMTKETVDAVCELIPSEERHEALRELMDLYLKMKPVWRSSC
PAKECPESLCQYSFNSQRFAELLSTKFKYRYEGKITNYFHKTLAHVPEIIERDGSIGA
WASEGNESGNKLFRRFRKMNARQSKCYEMEDVLKHHWLYTSKYLQKFMNAHNAL
KTSGFTMNPQASLGDP
Illustrative RAG1 polypeptide fragment 7
(SEQ ID NO: 13)
PRNVTMEWHPHTPSCDICNTARRGLKRKSLQPNLQLSKKLKTVLDQARQARQRKR
RAQARISSKDVMKKIANCSKIHLSTKLLAVDFPEHFVKSISCQICEHILADPVETNCKH
VFCRVCILRCLKVMGSYCPSCRYPCFPTDLESPVKSFLSVLNSLMVKCPAKECNEE
VSLEKYNHHISSHKESKEIFVHINKGGRPRQHLLSLTRRAQKHRLRELKLQVKAFAD
KEEGGDVKSVCMTLFLLALRARNEHRQADELEAIMQGKGSGLQPAVCLAIRVNTFL
SCSQYHKMYRTVKAITGRQIFQPLHALRNAEKVLLPGYHHFEWQPPLKNVSSSTDV
GIIDGLSGLSSSVDDYPVDTIAKRFRYDSALVSALMDMEEDILEGMRSQDLDDYLNG
PFTVVVKESCDGMGDVSEKHGSGPVVPEKAVRFSFTIMKITIAHSSQNVKVFEEAKP
NSELCCKPLCLMLADESDHETLTAILSPLIAEREAMKSSELMLELGGILRTFKFIFRGT
GYDEKLVREVEGLEASGSVYICTLCDATRLEASQNLVFHSITRSHAENLERYEVWR
SNPYHESVEELRDRVKGVSAKPFIETVPSIDALHCDIGNAAEFYKIFQLEIGEVYKNP
NASKEERKRWQATLDKHLRKKMNLKPIMRMNGNFARKLMTKETVDAVCELIPSEER
HEALRELMDLYLKMKPVWRSSCPAKECPESLCQYSFNSQRFAELLSTKFKYRYEG
KITNYFHKTLAHVPEIIERDGSIGAWASEGNESGNKLFRRFRKMNARQSKCYEMED
VLKHHWLYTSKYLQKFMNAHNALKTSGFTMNPQASLGDP
Illustrative RAG1 polypeptide fragment 8
(SEQ ID NO: 14)
LEKYNHHISSHKESKEIFVHINKGGRPRQHLLSLTRRAQKHRLRELKLQVKAFADKE
EGGDVKSVCMTLFLLALRARNEHRQADELEAIMQGKGSGLQPAVCLAIRVNTFLSC
SQYHKMYRTVKAITGRQIFQPLHALRNAEKVLLPGYHHFEWQPPLKNVSSSTDVGII
DGLSGLSSSVDDYPVDTIAKRFRYDSALVSALMDMEEDILEGMRSQDLDDYLNGPF
TVVVKESCDGMGDVSEKHGSGPVVPEKAVRFSFTIMKITIAHSSQNVKVFEEAKPN
SELCCKPLCLMLADESDHETLTAILSPLIAEREAMKSSELMLELGGILRTFKFIFRGTG
YDEKLVREVEGLEASGSVYICTLCDATRLEASQNLVFHSITRSHAENLERYEVWRSN
PYHESVEELRDRVKGVSAKPFIETVPSIDALHCDIGNAAEFYKIFQLEIGEVYKNPNA
SKEERKRWQATLDKHLRKKMNLKPIMRMNGNFARKLMTKETVDAVCELIPSEERHE
ALRELMDLYLKMKPVWRSSCPAKECPESLCQYSFNSQRFAELLSTKFKYRYEGKIT
NYFHKTLAHVPEIIERDGSIGAWASEGNESGNKLFRRFRKMNARQSKCYEMEDVLK
HHWLYTSKYLQKFMNAHNALKTSGFTMNPQASLGDP
Illustrative RAG1 polypeptide fragment 9
(SEQ ID NO: 164)
PRNVTMEWHPHTPSCDICNTARRGLKRKSLQPNLQLSKKLKTVLDQARQARQRKR
RAQARISSKDVMKKIANCSKIHLSTKLLAVDFPEHFVKSISCQICEHILADPVETNCKH
VFCRVCILRCLKVMGSYCPSCRYPCFPTDLESPVKSFLSVLNSLMVKCPAKECNEE
VSLEKYNHHISSHKESKEIFVHINKGGRPRQHLLSLTRRAQKHRLRELKLQVKAFAD
KEEGGDVKSVCMTLFLLALRARNEHRQADELEAIMQGKGSGLQPAVCLAIRVNTFL
SCSQYHKMYRTVKAITGRQIFQPLHALRNAEKVLLPGYHHFEWQPPLKNVSSSTDV
GIIDGLSGLSSSVDDYPVDTIAKRFRYDSALVSALMDMEEDILEGMRSQDLDDYLNG
PFTVVVKESCDGMGDVSEKHGSGPVVPEKAVRFSFTIMKITIAHSSQNVKVFEEAKP
NSELCCKPLCLMLADESDHETLTAILSPLIAEREAMKSSELMLELGGILRTFKFIFRGT
GYDEKLVREVEGLEASGSVYICTLCDATRLEASQNLVFHSITRSHAENLERYEVWR
SNPYHESVEELRDRVKGVSAKPFIETVPSIDALHCDIGNAAEFYKIFQLEIGEVYKNP
NASKEERKRWQATLDKHLRKKMNLKPIMRMNGNFARKLMTKETVDAVCELIPSEER
HEALRELMDLYLKMKPVWRSSCPAKECPESLCQYSFNSQRFAELLSTKFKYRYEG
KITNYFHKTLAHVPEIIERDGSIGAWASEGNESGNKLFRRFRKMNARQSKCYEMED
VLKHHWLYTSKYLQKFMNAHNALKTSGFTMNPQASLGDPLGIEDSLESQDSMEF
Illustrative RAG1 polypeptide fragment 10
(SEQ ID NO: 165)
EVYFPRNVTMEWHPHTPSCDICNTARRGLKRKSLQPNLQLSKKLKTVLDQARQAR
QRKRRAQARISSKDVMKKIANCSKIHLSTKLLAVDFPEHFVKSISCQICEHILADPVET
NCKHVFCRVCILRCLKVMGSYCPSCRYPCFPTDLESPVKSFLSVLNSLMVKCPAKE
CNEEVSLEKYNHHISSHKESKEIFVHINKGGRPRQHLLSLTRRAQKHRLRELKLQVK
AFADKEEGGDVKSVCMTLFLLALRARNEHRQADELEAIMQGKGSGLQPAVCLAIRV
NTFLSCSQYHKMYRTVKAITGRQIFQPLHALRNAEKVLLPGYHHFEWQPPLKNVSS
STDVGIIDGLSGLSSSVDDYPVDTIAKRFRYDSALVSALMDMEEDILEGMRSQDLDD
YLNGPFTVVVKESCDGMGDVSEKHGSGPVVPEKAVRFSFTIMKITIAHSSQNVKVF
EEAKPNSELCCKPLCLMLADESDHETLTAILSPLIAEREAMKSSELMLELGGILRTFK
FIFRGTGYDEKLVREVEGLEASGSVYICTLCDATRLEASQNLVFHSITRSHAENLERY
EVWRSNPYHESVEELRDRVKGVSAKPFIETVPSIDALHCDIGNAAEFYKIFQLEIGEV
YKNPNASKEERKRWQATLDKHLRKKMNLKPIMRMNGNFARKLMTKETVDAVCELIP
SEERHEALRELMDLYLKMKPVWRSSCPAKECPESLCQYSFNSQRFAELLSTKFKY
RYEGKITNYFHKTLAHVPEIIERDGSIGAWASEGNESGNKLFRRFRKMNARQSKCY
EMEDVLKHHWLYTSKYLQKFMNAHNALKTSGFTMNPQASLGDPLGIEDSLESQDS
MEF

### RAG1 Polynucleotides

A nucleotide sequence encoding a RAG1 polypeptide (or a variant of fragment thereof) may be codon-optimised. Suitably, a nucleotide sequence encoding a RAG1 polypeptide (or a variant of fragment thereof) may be codon optimised for expression in a human cell.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells (e.g. humans), as well as for a variety of other organisms.

In some embodiments of the invention, a nucleotide sequence encoding a RAG1 polypeptide comprises or consists of a nucleotide sequence which is at least 70% identical to SEQ ID NO: 15. Suitably, a nucleotide sequence encoding a RAG1 polypeptide comprises or consists of a nucleotide sequence which is at least 80%, at least 85%, at least 90%, at least 95%, at least 98% or at least 99% identical to SEQ ID NO: 15. In some embodiments of the invention, a nucleotide sequence encoding a RAG1 polypeptide comprises or consists of the nucleotide sequence SEQ ID NO: 15.

In some embodiments of the invention, the nucleotide sequence encoding a RAG1 polypeptide fragment comprises or consists of a nucleotide sequence which is at least 70% identical to a fragment of SEQ ID NO: 15. Suitably, the nucleotide sequence encoding a RAG1 polypeptide fragment comprises or consists of a nucleotide sequence which is at least 80%, at least 85%, at least 90%, at least 95%, at least 98% or at least 99% identical to a fragment of SEQ ID NO: 15. In some embodiments of the invention, a nucleotide sequence encoding a RAG1 polypeptide fragment comprises or consists of a fragment of the nucleotide sequence SEQ ID NO: 15.

Exemplary nucleotide sequence encoding a RAG1 polypeptide
(SEQ ID NO: 15)
atggccgcctccttcccacctacccttggattgtcctccgcccctgacgaaattcaacatccccacatcaaattctcgga
gtggaagttcaagctctttcgcgtgcgctcgttcgaaaagacccccgaggaagcccaaaaggagaagaaagactc
attcgaaggaaaacccagcctcgaacagtccccggccgtcctggacaaggccgacgggcagaagcctgtgccga
cccagccgctgctgaaagcgcacccgaaattctccaagaagtttcacgataacgagaaggcccggggaaaggcc
atccaccaagcaaaccttagacacctgtgccgcatctgtgggaactcattcagagccgacgaacataaccggagat
accctgtgcatggccctgtcgacggaaagaccctggggctcctgagaaagaaggagaagagggcgacatcctgg
ccggacctgatcgcaaaggtgttcagaatcgacgtgaaggcagatgtggacagcatccacccaaccgagttctgcc
acaactgctggagcattatgcaccggaagttcagctcagcgccctgtgaagtgtacttcccgcgcaacgtgactatgg
agtggcatccacacactccgtcctgcgacatctgtaacactgctcggcgcggactcaagaggaagtccctgcagccg
aatctgcagctgagcaagaagcttaagaccgtgctggaccaggctcggcaggcccgccagcacaagcgacgcgc
ccaggcccggatctcatctaaggatgtgatgaagaagatcgccaattgcagcaaaatccacctgtctaccaagctgct
ggcggtggacttcccggagcacttcgtgaagtccatcagctgtcagatctgcgagcatattctcgccgaccccgtgga
gactaattgcaagcacgtgttctgccgcgtgtgcatcctgcgctgcctgaaggtcatgggctcctattgcccttcctgccg
gtacccctgtttccctactgatctggagtccccggtcaagtccttcttgtccgtgctgaactccctgatggtcaaatgtcccg
caaaggagtgcaatgaggaagtgtccctggaaaagtacaaccaccacatcagcagccacaaggagtccaaaga
aatctttgtgcacattaacaagggcggtcggccccggcagcatctgctctcgctgactcgccgggcccagaagcaca
ggctccgggagctgaagctgcaagtcaaggccttcgccgacaaggaagagggaggagatgtgaagtccgtgtgca
tgaccctgtttttgctggcgctgcgggctcggaacgaacacagacaagctgatgaactggaggccatcatgcagggc
aaaggatcgggactccagccggctgtgtgtctcgccatccgcgtcaacacattcctctcatgctcccaataccacaag
atgtacaggactgtgaaggccatcaccggacggcagatctttcagccactccacgcccttcggaacgcagaaaagg
tcttgctgccgggataccatcatttcgaatggcagccgcccttgaaaaacgtgtcctcgtccaccgacgtgggcattatt
gatgggctgagcggcctgtcctcctctgtggatgactaccctgtggataccatcgccaaacggttcagatacgattccg
cgctggtgtcggccctgatggacatggaggaggacatcctggagggaatgagatcacaagatctggacgactacct
caacgggcccttcacggtggtggtcaaggaatcgtgcgatggaatgggcgacgtgtcggagaagcacggttccgga
cctgtggtgccggaaaaggccgtgcgcttctccttcaccatcatgaagatcaccattgcgcatagctcccagaacgtca
aagtgttcgaagaggccaagccgaactcagagctctgctgcaagccgctgtgcctgatgttggcggacgagagcga
tcacgaaaccctgaccgccattctgtcgcctctgatcgcggagagggaggccatgaagtcctccgaactgatgctgg
agctgggcggtattttgcggacttttaagttcatcttccggggaaccggttatgacgaaaagctcgtgcgcgaagtgga
gggcctggaagcctcaggctccgtctacatctgcactctctgcgacgccacccggctggaggcgtcacagaatcttgt
gttccactcgatcactaggtcccacgcggagaacctggaacgctatgaggtctggcgctctaacccataccacgaatc
cgtggaagaacttcgggacagagtgaagggagtgtcagcaaagcctttcattgaaaccgtgcctagcatcgacgcc
ctccattgcgacatcggcaacgccgccgagttctacaagatcttccagcttgagatcggggaagtgtacaagaaccc
gaacgcctccaaggaagaaagaaagcggtggcaggctacccttgacaaacacctccgcaagaagatgaacctg
aagcccattatgcggatgaacggaaacttcgctaggaagctgatgactaaggaaacggtcgacgcggtctgtgaact
gatccccagcgaagaacgacatgaagcgctgcgcgaactcatggacctgtacctgaagatgaagcctgtctggcgg
agctcgtgccctgccaaggagtgcccggagtcgctgtgtcagtacagctttaacagccaaaggttcgcagagctgctg
tcgaccaagttcaagtacagatacgaaggaaagattaccaactacttccacaagactctcgctcacgtgcccgagatt
atcgaacgcgatggttccatcggggcctgggcctccgagggcaacgagtcgggcaacaagttgttccgccggtttag
aaagatgaacgcccgccagtccaagtgctacgaaatggaagatgtgctgaagcatcactggctgtatacctccaagt
acctccagaagttcatgaacgcacataacgccctcaagacctccgggttcaccatgaacccccaggcctccctcggt
gaccctctgggaattgaagatagcttggagagccaggactcgatggaattcta

In some embodiments of the invention, a nucleotide sequence encoding a RAG1 polypeptide comprises or consists of a nucleotide sequence which is at least 70% identical to SEQ ID NO: 16. Suitably, a nucleotide sequence encoding a RAG1 polypeptide comprises or consists of a nucleotide sequence which is at least 80%, at least 85%, at least 90%, at least 95%, at least 98% or at least 99% identical to SEQ ID NO: 16. In some embodiments of the invention, a nucleotide sequence encoding a RAG1 polypeptide comprises or consists of the nucleotide sequence SEQ ID NO: 16.

In some embodiments of the invention, the nucleotide sequence encoding a RAG1 polypeptide fragment comprises or consists of a nucleotide sequence which is at least 70% identical to a fragment of SEQ ID NO: 16. Suitably, the nucleotide sequence encoding a RAG1 polypeptide fragment comprises or consists of a nucleotide sequence which is at least 80%, at least 85%, at least 90%, at least 95%, at least 98% or at least 99% identical to a fragment of SEQ ID NO: 16. In some embodiments of the invention, a nucleotide sequence encoding a RAG1 polypeptide fragment comprises or consists of a fragment of the nucleotide sequence SEQ ID NO: 16.

Illustrative nucleotide sequence encoding a RAG1 polypeptide
(SEQ ID NO: 16)
atggccgccagctttcctcctacactgggactgtctagcgcccctgacgagattcagcaccctcacatcaagttcagcg
agtggaagttcaagctgttcagagtgcggagcttcgagaaaacccctgaggaagcccagaaagagaagaaggac
agcttcgagggcaagcccagcctggaacagtctcctgctgtgctggataaggccgacggccagaaacctgtgccta
cacagcctctgctgaaggctcaccccaagttctccaagaagttccacgacaacgagaaggccagaggcaaggcc
atccaccaggccaatctgagacacctgtgccggatctgcggcaacagcttcagagccgacgagcacaatcggaga
taccctgtgcacggccctgtggatggaaagactctgggcctgctgcggaagaaagaaaagagagccaccagctgg
cccgacctgatcgccaaggtgttcagaatcgacgtgaaggccgatgtggacagcattcaccccaccgagttctgcca
caactgctggtccatcatgcaccggaagttcagctctgccccttgcgaggtgtacttccccagaaacgtgaccatgga
atggcacccacacacacccagctgcgacatctgcaacacagccagaagaggcctgaagcggaagtccctgcagc
ctaatctgcagctgagcaagaaactgaaaaccgtgctggaccaggccagacaggcccggcaaagaaaaagacg
cgcccaggctagaatcagcagcaaggacgtgatgaagaagatcgccaactgcagcaagatccacctgagcacca
aactgctggccgtggacttccctgagcacttcgtgaagtccatcagctgccagatctgcgagcacatcctggccgatcc
tgtggaaacaaactgcaagcacgtgttctgcagagtgtgcatcctgcggtgcctgaaagtgatgggcagctactgccc
cagctgtagatacccttgcttccccaccgacctggaaagccctgtgaagtcctttctgagcgtgctgaacagcctgatg
gtcaagtgccccgccaaagaatgcaacgaggaagtgtccctggaaaagtacaaccaccacatcagcagccacaa
agagtccaaagaaatcttcgtgcacatcaacaaaggcggcagaccccggcagcatctgctgtctcttacaagaagg
gcccagaagcaccggctgcgggaactgaagctgcaagtgaaggcctttgccgacaaagaggaaggcggcgacg
tcaagagcgtgtgcatgaccctgtttctgctggccctgagagcccggaatgagcatagacaggccgatgagctggaa
gccattatgcaaggcaaaggcagcggactgcagcctgctgtgtgtctggctatcagagtgaataccttcctgagctgc
agccagtaccacaagatgtaccggaccgtgaaagccatcaccggcagacagatcttccagccactgcacgccctg
agaaacgccgagaaagttctgctgcctggctaccaccacttcgagtggcagcctccactgaagaacgtgtccagca
gcaccgacgtgggcatcatcgatggactgtctggactgagcagcagcgtggacgattaccccgtggacacaatcgc
caagagattcagatacgacagcgccctggtgtctgccctgatggacatggaagaggacatcctggaaggcatgcgg
agccaggacctggacgactatctgaacggccctttcaccgtggtggtcaaagaaagctgtgacggcatgggcgacg
tgtccgagaaacacggatctggacctgtggtgccagagaaggccgtgcggttcagcttcaccatcatgaagatcact
atcgcccacagcagccagaacgtgaaggtgttcgaggaagccaagcctaacagcgagctgtgctgcaagcctctgt
gtctgatgctggccgacgagagcgatcacgagacactgaccgccattctgagccctctgatcgccgaacgggaagc
catgaagtcctccgagctgatgctcgaactcggcggcatcctgagaaccttcaagttcatcttccgcggcaccggctac
gacgagaagctcgttagagaggtggaaggcctggaagcctctggcagcgtgtacatctgcaccctgtgtgacgcca
ccagactggaagctagccagaacctggtgttccacagcatcaccagaagccacgccgaaaacctcgagagatac
gaagtgtggcggagcaacccctaccacgagagcgtggaagaactgcgggatagagtgaagggcgtgtccgccaa
gcctttcatcgagacagtgcctagcatcgacgccctgcactgcgatattggcaacgccgccgaattctacaagatctttc
agctggaaatcggcgaggtctacaagaaccccaacgcctctaaagaggaacggaagcgctggcaggccacactg
gataagcacctgagaaagaagatgaatctgaagcccatcatgcggatgaacggcaacttcgcccggaagctgatg
accaaagaaaccgtggatgccgtgtgcgagctgatcccctctgaggaaagacacgaggccctgagggaactgatg
gacctgtacctgaagatgaagcccgtgtggcggtctagctgtcctgccaaagagtgccctgagtctctgtgccagtaca
gcttcaacagccagagattcgccgagctgctgagcacaaagttcaagtacagatacgaggggaagatcacgaact
acttccacaagaccctggctcacgtgcccgagatcatcgagagagatggctctattggcgcctgggcctctgagggc
aatgagtctggcaacaagctgtttcggcggttcagaaagatgaacgccagacagagcaagtgctacgagatggaa
gatgtgctgaagcaccactggctgtacaccagcaagtacctgcagaaattcatgaacgcccacaacgccctcaaga
ccagcggctttaccatgaatcctcaggccagcctgggagatcctctgggcattgaggatagcctggaatcccaggac
agcatggaattctga

Suitably, a nucleotide sequence encoding a RAG1 polypeptide fragment may be at least 100 bp in length, 200 bp in length, 300 bp in length, 400 bp in length, 500 bp in length, 600 bp in length, 700 bp in length, 800 bp in length, 900 bp in length, 1000 bp in length, 1100 bp in length, 1200 bp in length, 1300 bp in length, 1400 bp in length, 1500 bp in length, at least 1600 bp in length, at least 1700 bp in length, at least 1800 bp in length, at least 1900 bp in length, at least 2000 bp in length, at least 2100 bp in length, at least 2200 bp in length, at least 2300 bp in length, at least 2400 bp in length, at least 2500 bp in length, at least 2600 bp in length, at least 2700 bp in length, at least 2800 bp in length, at least 2900 bp in length, or at least 3000 bp in length.

Suitably, a nucleotide sequence encoding a RAG1 polypeptide fragment may comprise at least the final 200 bp, at least the final 300 bp, at least the final 400 bp, at least the final 500 bp, at least the final 600 bp, at least the final 700 bp, at least the final 800 bp, at least the final 900 bp, at least the final 1000 bp, at least the final 1100 bp, at least the final 1200 bp, at least the final 1300 bp, at least the final 1400 bp, at least the final 1500 bp, at least the final 1600 bp, at least the final 1700 bp, at least the final 1800 bp, at least the final 1900 bp, at least the final 2000 bp, at least the final 2100 bp, at least the final 2200 bp, at least the final 2300 bp, at least the final 2400 bp, at least the final 2500 bp, at least the final 2600 bp, at least the final 2700 bp, at least the final 2800 bp, at least the final 2900 bp, at least the final 3000 bp of a full-length RAG1 nucleotide or variant thereof, optionally wherein 1 to 100 bp (e.g. about 50 bp) are absent from the 3′-end of the full-length RAG1 nucleotide or variant thereof.

A nucleotide sequence encoding a RAG1 polypeptide fragment may be in-frame with the RAG1 gene. A person skilled in the art would be able to generate nucleotide sequences encoding a RAG1 polypeptide fragment which are in-frame with the RAG1 gene using techniques known in the art.

A nucleotide sequence encoding a RAG1 polypeptide fragment may be used replace part of the RAG1 gene which encodes an endogenous RAG1 polypeptide. The nucleotide sequence encoding a RAG1 polypeptide fragment may be introduced in-frame with the remaining part of the RAG1 gene. For example, a nucleotide sequence encoding a downstream portion of the RAG1 polypeptide fragment may be introduced into the RAG1 exon 2 in-frame with an upstream portion of the endogenous RAG1 gene, such that the edited RAG1 gene encodes a RAG1 polypeptide.

In some embodiments of the invention, the nucleotide sequence encoding a RAG1 polypeptide fragment comprises or consists of a nucleotide sequence which is at least 70% identical any of SEQ ID NOs: 17-24 or 158-159. Suitably, the nucleotide sequence encoding a RAG1 polypeptide fragment comprises or consists of a nucleotide sequence which is at least 80%, at least 85%, at least 90%, at least 95%, at least 98% or at least 99% identical to any of SEQ ID NOs: 17-24 or 158-159.

In some embodiments of the invention, a nucleotide sequence encoding a RAG1 polypeptide fragment comprises or consists of the nucleotide sequence of any of SEQ ID NOs: 17-24 or 158-159.

Illustrative nucleotide encoding a RAG1 polypeptide fragment 1
(SEQ ID NO: 17)
gcagcaagatccacctgagcaccaaactgctggccgtggacttccctgagcacttcgtgaagtccatcagctgccag
atctgcgagcacatcctggccgatcctgtggaaacaaactgcaagcacgtgttctgcagagtgtgcatcctgcggtgc
ctgaaagtgatgggcagctactgcccctcctgcagatacccttgcttccccaccgatctggaaagccctgtgaagtcctt
cctgagcgtgctgaacagcctgatggtcaagtgccccgccaaagaatgcaacgaggaagtgtccctggaaaagta
caaccaccacatcagcagccacaaagagtccaaagaaatcttcgtgcacatcaacaaaggcggcagaccccggc
agcatctgctgtctcttacaagacgggcccagaagcaccggctgagagaactgaagctgcaagtgaaggcctttgcc
gacaaagaggaaggcggcgacgtcaagagcgtgtgcatgaccctgtttctgctggccctgagagcccggaatgag
catagacaggccgatgagctggaagccatcatgcaaggcaaaggcagcggactgcagcctgctgtgtgtctggcta
tcagagtgaacaccttcctgtcctgcagccagtaccacaagatgtaccggaccgtgaaggccattaccggcagaca
gatcttccagcctctgcacgccctgagaaacgccgagaaagttctgctgcctggctaccaccacttcgagtggcagcc
tccactgaagaacgtgtccagcagcaccgacgtgggcatcatcgatggactgagcggactgtctagcagcgtggac
gactaccccgtggacacaatcgccaagcggttcagatacgacagcgccctggtgtctgccctgatggacatggaag
aggacatcctggaaggcatgcggagccaggacctggacgattacctgaacggccctttcaccgtggtggtcaaaga
aagctgtgacggcatgggcgacgtgtccgagaaacacggatctggacctgtggtgccagagaaggccgtgcggttc
agcttcaccatcatgaagatcactatcgcccacagcagccagaacgtgaaagtgttcgaggaagccaagcctaaca
gcgagctgtgctgcaagcctctgtgtctgatgctggccgacgagagcgatcacgagacactgaccgccattctgagc
cctctgatcgccgaacgggaagccatgaagtcctccgagctgatgctcgaactcggcggcatcctgagaaccttcaa
gttcatcttccgcggcaccggctacgacgagaagctcgttagagaggtggaaggcctggaagcctctggcagcgtgt
acatctgcaccctgtgtgacgccaccagactggaagctagccagaacctggtgttccacagcatcaccagaagcca
cgccgaaaacctggaaagatacgaagtgtggcggagcaacccctaccacgagagcgtggaagaactgcgggat
agagtgaagggcgtgtccgccaagcctttcatcgagacagtgcctagcatcgacgccctgcactgcgatattggcaa
cgccgccgaattctacaagatctttcagctggaaatcggcgaggtgtacaagaaccccaacgcctctaaagaggaa
cggaagcgctggcaggccacactggataagcacctgagaaagaagatgaatctgaagcccatcatgaggatgaa
cggcaacttcgcccggaagctgatgaccaaagaaaccgtggatgccgtgtgcgagctgatcccctctgaggaaaga
cacgaggccctgcgggaactgatggacctgtacctgaagatgaagcccgtgtggcggtctagctgtcctgccaaaga
gtgccctgagtctctgtgccagtacagcttcaacagccagagattcgccgagctgctgtccaccaagttcaagtacag
atacgagggcaagatcaccaactacttccacaagaccctggctcacgtgcccgagatcatcgagagagatggctct
attggcgcctgggcctctgagggcaatgagtctggcaacaagctgttccggcggttccgcaagatgaacgccagaca
gagcaagtgctacgagatggaagatgtgctgaagcaccactggctgtacaccagcaagtacctgcagaaattcatg
aacgcccacaacgccctcaagaccagcggctttaccatgaatcctcaggccagcctgggcgaccctctgggaattg
aggatagcctggaatcccaggacagcatggaattctga
Illustrative nucleotide encoding a RAG1 polypeptide fragment 2
(SEQ ID NO: 18)
gcagcaagatccacctgagcaccaaactgctggccgtggacttccctgagcacttcgtgaagtccatcagctgccag
atctgcgagcacatcctggccgatcctgtggaaacaaactgcaagcacgtgttctgcagagtgtgcatcctgcggtgc
ctgaaagtgatgggcagctactgcccctcctgcagatacccttgcttccccaccgatctggaaagccctgtgaagtcctt
cctgagcgtgctgaacagcctgatggtcaagtgccccgccaaagaatgcaacgaggaagtgtccctggaaaagta
caaccaccacatcagcagccacaaagagtccaaagaaatcttcgtgcacatcaacaaaggggcagaccccggc
agcatctgctgtctcttacaagacgggcccagaagcaccggctgagagaactgaagctgcaagtgaaggcctttgcc
gacaaagaggaaggcggcgacgtcaagagcgtgtgcatgaccctgtttctgctggccctgagagcccggaatgag
catagacaggccgatgagctggaagccatcatgcaaggcaaaggcagcggactgcagcctgctgtgtgtctggcta
tcagagtgaacaccttcctgtcctgcagccagtaccacaagatgtaccggaccgtgaaggccattaccggcagaca
gatcttccagcctctgcacgccctgagaaacgccgagaaagttctgctgcctggctaccaccacttcgagtggcagcc
tccactgaagaacgtgtccagcagcaccgacgtgggcatcatcgatggactgagcggactgtctagcagcgtggac
gactaccccgtggacacaatcgccaagcggttcagatacgacagcgccctggtgtctgccctgatggacatggaag
aggacatcctggaaggcatgcggagccaggacctggacgattacctgaacggccctttcaccgtggtggtcaaaga
aagctgtgacggcatgggcgacgtgtccgagaaacacggatctggacctgtggtgccagagaaggccgtgcggttc
agcttcaccatcatgaagatcactatcgcccacagcagccagaacgtgaaagtgttcgaggaagccaagcctaaca
gcgagctgtgctgcaagcctctgtgtctgatgctggccgacgagagcgatcacgagacactgaccgccattctgagc
cctctgatcgccgaacgggaagccatgaagtcctccgagctgatgctcgaactcggcggcatcctgagaaccttcaa
gttcatcttccgcggcaccggctacgacgagaagctcgttagagaggtggaaggcctggaagcctctggcagcgtgt
acatctgcaccctgtgtgacgccaccagactggaagctagccagaacctggtgttccacagcatcaccagaagcca
cgccgaaaacctggaaagatacgaagtgtggcggagcaacccctaccacgagagcgtggaagaactgcgggat
agagtgaagggcgtgtccgccaagcctttcatcgagacagtgcctagcatcgacgccctgcactgcgatattggcaa
cgccgccgaattctacaagatctttcagctggaaatcggcgaggtgtacaagaaccccaacgcctctaaagaggaa
cggaagcgctggcaggccacactggataagcacctgagaaagaagatgaatctgaagcccatcatgaggatgaa
cggcaacttcgcccggaagctgatgaccaaagaaaccgtggatgccgtgtgcgagctgatcccctctgaggaaaga
cacgaggccctgcgggaactgatggacctgtacctgaagatgaagcccgtgtggcggtctagctgtcctgccaaaga
gtgccctgagtctctgtgccagtacagcttcaacagccagagattcgccgagctgctgtccaccaagttcaagtacag
atacgagggcaagatcaccaactacttccacaagaccctggctcacgtgcccgagatcatcgagagagatggctct
attggcgcctgggcctctgagggcaatgagtctggcaacaagctgttccggcggttccgcaagatgaacgccagaca
gagcaagtgctacgagatggaagatgtgctgaagcaccactggctgtacaccagcaagtacctgcagaaattcatg
aacgcccacaacgccctcaagaccagcggctttaccatgaatcctcaggccagcctgggcgatccttt
Illustrative nucleotide encoding a RAG1 polypeptide fragment 3
(SEQ ID NO: 19)
gaatggcaccctcacacacccagctgcgacatctgcaacacagccagaagaggcctgaagcggaagtccctgca
gcctaatctgcagctgagcaagaaactgaaaaccgtgctggaccaggccagacaggcccggcaaagaaagaga
agggcccaagccagaatcagcagcaaggacgtgatgaagaagatcgccaactgcagcaagatccacctgagca
ccaaactgctggccgtggacttccctgagcacttcgtgaagtccatcagctgccagatctgcgagcacatcctggccg
atcctgtggaaacaaactgcaagcacgtgttctgcagagtgtgcatcctgcggtgcctgaaagtgatgggcagctact
gcccctcctgcagatacccttgcttccccaccgatctggaaagccctgtgaagtccttcctgagcgtgctgaacagcct
gatggtcaagtgccccgccaaagaatgcaacgaggaagtgtccctggaaaagtacaaccaccacatcagcagcc
acaaagagtccaaagaaatcttcgtgcacatcaacaaaggcggcagaccccggcagcatctgctgtctcttacaag
acgggcccagaagcaccggctgagagaactgaagctgcaagtgaaggcctttgccgacaaagaggaaggcggc
gacgtcaagagcgtgtgcatgaccctgtttctgctggccctgagagcccggaatgagcatagacaggccgatgagct
ggaagccatcatgcaaggcaaaggcagcggactgcagcctgctgtgtgtctggctatcagagtgaacaccttcctgtc
ctgcagccagtaccacaagatgtaccggaccgtgaaggccattaccggcagacagatcttccagcctctgcacgcc
ctgagaaacgccgagaaagttctgctgcctggctaccaccacttcgagtggcagcctccactgaagaacgtgtccag
cagcaccgacgtgggcatcatcgatggactgagcggactgtctagcagcgtggacgactaccccgtggacacaatc
gccaagcggttcagatacgacagcgccctggtgtctgccctgatggacatggaagaggacatcctggaaggcatgc
ggagccaggacctggacgattacctgaacggccctttcaccgtggtggtcaaagaaagctgtgacggcatgggcga
cgtgtccgagaaacacggatctggacctgtggtgccagagaaggccgtgcggttcagcttcaccatcatgaagatca
ctatcgcccacagcagccagaacgtgaaagtgttcgaggaagccaagcctaacagcgagctgtgctgcaagcctct
gtgtctgatgctggccgacgagagcgatcacgagacactgaccgccattctgagccctctgatcgccgaacgggaa
gccatgaagtcctccgagctgatgctcgaactcggcggcatcctgagaaccttcaagttcatcttccgcggcaccggct
acgacgagaagctcgttagagaggtggaaggcctggaagcctctggcagcgtgtacatctgcaccctgtgtgacgc
caccagactggaagctagccagaacctggtgttccacagcatcaccagaagccacgccgaaaacctggaaagat
acgaagtgtggcggagcaacccctaccacgagagcgtggaagaactgcgggatagagtgaagggcgtgtccgcc
aagcctttcatcgagacagtgcctagcatcgacgccctgcactgcgatattggcaacgccgccgaattctacaagatc
tttcagctggaaatcggcgaggtgtacaagaaccccaacgcctctaaagaggaacggaagcgctggcaggccac
actggataagcacctgagaaagaagatgaatctgaagcccatcatgaggatgaacggcaacttcgcccggaagct
gatgaccaaagaaaccgtggatgccgtgtgcgagctgatcccctctgaggaaagacacgaggccctgcgggaact
gatggacctgtacctgaagatgaagcccgtgtggcggtctagctgtcctgccaaagagtgccctgagtctctgtgccag
tacagcttcaacagccagagattcgccgagctgctgtccaccaagttcaagtacagatacgagggcaagatcacca
actacttccacaagaccctggctcacgtgcccgagatcatcgagagagatggctctattggcgcctgggcctctgagg
gcaatgagtctggcaacaagctgttccggcggttccgcaagatgaacgccagacagagcaagtgctacgagatgg
aagatgtgctgaagcaccactggctgtacaccagcaagtacctgcagaaattcatgaacgcccacaacgccctcaa
gaccagcggctttaccatgaatcctcaggccagcctgggcgaccctctgggaattgaggatagcctggaatcccagg
acagcatggaattctgataa
Illustrative nucleotide encoding a RAG1 polypeptide fragment 4
(SEQ ID NO: 20)
gaatggcaccctcacacacccagctgcgacatctgcaacacagccagaagaggcctgaagcggaagtccctgca
gcctaatctgcagctgagcaagaaactgaaaaccgtgctggaccaggccagacaggcccggcaaagaaagaga
agggcccaagccagaatcagcagcaaggacgtgatgaagaagatcgccaactgcagcaagatccacctgagca
ccaaactgctggccgtggacttccctgagcacttcgtgaagtccatcagctgccagatctgcgagcacatcctggccg
atcctgtggaaacaaactgcaagcacgtgttctgcagagtgtgcatcctgcggtgcctgaaagtgatgggcagctact
gcccctcctgcagatacccttgcttccccaccgatctggaaagccctgtgaagtccttcctgagcgtgctgaacagcct
gatggtcaagtgccccgccaaagaatgcaacgaggaagtgtccctggaaaagtacaaccaccacatcagcagcc
acaaagagtccaaagaaatcttcgtgcacatcaacaaaggcggcagaccccggcagcatctgctgtctcttacaag
acgggcccagaagcaccggctgagagaactgaagctgcaagtgaaggcctttgccgacaaagaggaaggcggc
gacgtcaagagcgtgtgcatgaccctgtttctgctggccctgagagcccggaatgagcatagacaggccgatgagct
ggaagccatcatgcaaggcaaaggcagcggactgcagcctgctgtgtgtctggctatcagagtgaacaccttcctgtc
ctgcagccagtaccacaagatgtaccggaccgtgaaggccattaccggcagacagatcttccagcctctgcacgcc
ctgagaaacgccgagaaagttctgctgcctggctaccaccacttcgagtggcagcctccactgaagaacgtgtccag
cagcaccgacgtgggcatcatcgatggactgagcggactgtctagcagcgtggacgactaccccgtggacacaatc
gccaagcggttcagatacgacagcgccctggtgtctgccctgatggacatggaagaggacatcctggaaggcatgc
ggagccaggacctggacgattacctgaacggccctttcaccgtggtggtcaaagaaagctgtgacggcatgggcga
cgtgtccgagaaacacggatctggacctgtggtgccagagaaggccgtgcggttcagcttcaccatcatgaagatca
ctatcgcccacagcagccagaacgtgaaagtgttcgaggaagccaagcctaacagcgagctgtgctgcaagcctct
gtgtctgatgctggccgacgagagcgatcacgagacactgaccgccattctgagccctctgatcgccgaacgggaa
gccatgaagtcctccgagctgatgctcgaactcggcggcatcctgagaaccttcaagttcatcttccgcggcaccggct
acgacgagaagctcgttagagaggtggaaggcctggaagcctctggcagcgtgtacatctgcaccctgtgtgacgc
caccagactggaagctagccagaacctggtgttccacagcatcaccagaagccacgccgaaaacctggaaagat
acgaagtgtggcggagcaacccctaccacgagagcgtggaagaactgcgggatagagtgaagggcgtgtccgcc
aagcctttcatcgagacagtgcctagcatcgacgccctgcactgcgatattggcaacgccgccgaattctacaagatc
tttcagctggaaatcggcgaggtgtacaagaaccccaacgcctctaaagaggaacggaagcgctggcaggccac
actggataagcacctgagaaagaagatgaatctgaagcccatcatgaggatgaacggcaacttcgcccggaagct
gatgaccaaagaaaccgtggatgccgtgtgcgagctgatcccctctgaggaaagacacgaggccctgcgggaact
gatggacctgtacctgaagatgaagcccgtgtggcggtctagctgtcctgccaaagagtgccctgagtctctgtgccag
tacagcttcaacagccagagattcgccgagctgctgtccaccaagttcaagtacagatacgagggcaagatcacca
actacttccacaagaccctggctcacgtgcccgagatcatcgagagagatggctctattggcgcctgggcctctgagg
gcaatgagtctggcaacaagctgttccggcggttccgcaagatgaacgccagacagagcaagtgctacgagatgg
aagatgtgctgaagcaccactggctgtacaccagcaagtacctgcagaaattcatgaacgcccacaacgccctcaa
gaccagcggctttaccatgaatcctcaggccagcctgggcgatccttt
Illustrative nucleotide encoding a RAG1 polypeptide fragment 5
(SEQ ID NO: 21)
gcgaagtgtacttccccagaaacgtgaccatggaatggcaccctcacacacccagctgcgacatctgcaacacagc
cagaagaggcctgaagcggaagtccctgcagcctaatctgcagctgagcaagaaactgaaaaccgtgctggacc
aggccagacaggcccggcaaagaaagagaagggcccaagccagaatcagcagcaaggacgtgatgaagaag
atcgccaactgcagcaagatccacctgagcaccaaactgctggccgtggacttccctgagcacttcgtgaagtccatc
agctgccagatctgcgagcacatcctggccgatcctgtggaaacaaactgcaagcacgtgttctgcagagtgtgcatc
ctgcggtgcctgaaagtgatgggcagctactgcccctcctgcagatacccttgcttccccaccgatctggaaagccctg
tgaagtccttcctgagcgtgctgaacagcctgatggtcaagtgccccgccaaagaatgcaacgaggaagtgtccctg
gaaaagtacaaccaccacatcagcagccacaaagagtccaaagaaatcttcgtgcacatcaacaaaggcggcag
accccggcagcatctgctgtctcttacaagacgggcccagaagcaccggctgagagaactgaagctgcaagtgaa
ggcctttgccgacaaagaggaaggcggcgacgtcaagagcgtgtgcatgaccctgtttctgctggccctgagagccc
ggaatgagcatagacaggccgatgagctggaagccatcatgcaaggcaaaggcagcggactgcagcctgctgtgt
gtctggctatcagagtgaacaccttcctgtcctgcagccagtaccacaagatgtaccggaccgtgaaggccattaccg
gcagacagatcttccagcctctgcacgccctgagaaacgccgagaaagttctgctgcctggctaccaccacttcgagt
ggcagcctccactgaagaacgtgtccagcagcaccgacgtgggcatcatcgatggactgagcggactgtctagcag
cgtggacgactaccccgtggacacaatcgccaagcggttcagatacgacagcgccctggtgtctgccctgatggac
atggaagaggacatcctggaaggcatgcggagccaggacctggacgattacctgaacggccctttcaccgtggtgg
tcaaagaaagctgtgacggcatgggcgacgtgtccgagaaacacggatctggacctgtggtgccagagaaggccg
tgcggttcagcttcaccatcatgaagatcactatcgcccacagcagccagaacgtgaaagtgttcgaggaagccaag
cctaacagcgagctgtgctgcaagcctctgtgtctgatgctggccgacgagagcgatcacgagacactgaccgccat
tctgagccctctgatcgccgaacgggaagccatgaagtcctccgagctgatgctcgaactcggcggcatcctgagaa
ccttcaagttcatcttccgcggcaccggctacgacgagaagctcgttagagaggtggaaggcctggaagcctctggc
agcgtgtacatctgcaccctgtgtgacgccaccagactggaagctagccagaacctggtgttccacagcatcaccag
aagccacgccgaaaacctggaaagatacgaagtgtggcggagcaacccctaccacgagagcgtggaagaactg
cgggatagagtgaagggcgtgtccgccaagcctttcatcgagacagtgcctagcatcgacgccctgcactgcgatatt
ggcaacgccgccgaattctacaagatctttcagctggaaatcggcgaggtctacaagaaccccaacgcctctaaag
aggaacggaagcgctggcaggccacactggataagcacctgagaaagaagatgaatctgaagcccatcatgagg
atgaacggcaacttcgcccggaagctgatgaccaaagaaaccgtggatgccgtgtgcgagctgatcccctctgagg
aaagacacgaggccctgcgggaactgatggacctgtacctgaagatgaagcccgtgtggcggtctagctgtcctgcc
aaagagtgccctgagtctctgtgccagtacagcttcaacagccagagattcgccgagctgctgtccaccaagttcaag
tacagatacgagggcaagatcaccaactacttccacaagaccctggctcacgtgcccgagatcatcgagagagatg
gctctattggcgcctgggcctctgagggcaatgagtctggcaacaagctgttccggcggttccgcaagatgaacgcca
gacagagcaagtgctacgagatggaagatgtgctgaagcaccactggctgtacaccagcaagtacctgcagaaatt
catgaacgcccacaacgccctcaagaccagcggctttaccatgaatcctcaggccagcctgggcgatccttt
Illustrative nucleotide encoding a RAG1 polypeptide fragment 6
(SEQ ID NO: 22)
tagaagaggcctgaagcggaagtccctgcagcctaatctgcagctgagcaagaaactgaaaaccgtgctggacca
ggccagacaggcccggcaaagaaagagaagggcccaagccagaatcagcagcaaggacgtgatgaagaaga
tcgccaactgcagcaagatccacctgagcaccaaactgctggccgtggacttccctgagcacttcgtgaagtccatca
gctgccagatctgcgagcacatcctggccgatcctgtggaaacaaactgcaagcacgtgttctgcagagtgtgcatcc
tgcggtgcctgaaagtgatgggcagctactgcccctcctgcagatacccttgcttccccaccgatctggaaagccctgt
gaagtccttcctgagcgtgctgaacagcctgatggtcaagtgccccgccaaagaatgcaacgaggaagtgtccctgg
aaaagtacaaccaccacatcagcagccacaaagagtccaaagaaatcttcgtgcacatcaacaaaggcggcaga
ccccggcagcatctgctgtctcttacaagacgggcccagaagcaccggctgagagaactgaagctgcaagtgaag
gcctttgccgacaaagaggaaggcggcgacgtcaagagcgtgtgcatgaccctgtttctgctggccctgagagcccg
gaatgagcatagacaggccgatgagctggaagccatcatgcaaggcaaaggcagcggactgcagcctgctgtgtg
tctggctatcagagtgaacaccttcctgtcctgcagccagtaccacaagatgtaccggaccgtgaaggccattaccgg
cagacagatcttccagcctctgcacgccctgagaaacgccgagaaagttctgctgcctggctaccaccacttcgagtg
gcagcctccactgaagaacgtgtccagcagcaccgacgtgggcatcatcgatggactgagcggactgtctagcagc
gtggacgactaccccgtggacacaatcgccaagcggttcagatacgacagcgccctggtgtctgccctgatggacat
ggaagaggacatcctggaaggcatgcggagccaggacctggacgattacctgaacggccctttcaccgtggtggtc
aaagaaagctgtgacggcatgggcgacgtgtccgagaaacacggatctggacctgtggtgccagagaaggccgtg
cggttcagcttcaccatcatgaagatcactatcgcccacagcagccagaacgtgaaagtgttcgaggaagccaagc
ctaacagcgagctgtgctgcaagcctctgtgtctgatgctggccgacgagagcgatcacgagacactgaccgccatt
ctgagccctctgatcgccgaacgggaagccatgaagtcctccgagctgatgctcgaactcggcggcatcctgagaa
ccttcaagttcatcttccgcggcaccggctacgacgagaagctcgttagagaggtggaaggcctggaagcctctggc
agcgtgtacatctgcaccctgtgtgacgccaccagactggaagctagccagaacctggtgttccacagcatcaccag
aagccacgccgaaaacctggaaagatacgaagtgtggcggagcaacccctaccacgagagcgtggaagaactg
cgggatagagtgaagggcgtgtccgccaagcctttcatcgagacagtgcctagcatcgacgccctgcactgcgatatt
ggcaacgccgccgaattctacaagatctttcagctggaaatcggcgaggtctacaagaaccccaacgcctctaaag
aggaacggaagcgctggcaggccacactggataagcacctgagaaagaagatgaatctgaagcccatcatgagg
atgaacggcaacttcgcccggaagctgatgaccaaagaaaccgtggatgccgtgtgcgagctgatcccctctgagg
aaagacacgaggccctgcgggaactgatggacctgtacctgaagatgaagcccgtgtggcggtctagctgtcctgcc
aaagagtgccctgagtctctgtgccagtacagcttcaacagccagagattcgccgagctgctgtccaccaagttcaag
tacagatacgagggcaagatcaccaactacttccacaagaccctggctcacgtgcccgagatcatcgagagagatg
gctctattggcgcctgggcctctgagggcaatgagtctggcaacaagctgttccggcggttccgcaagatgaacgcca
gacagagcaagtgctacgagatggaagatgtgctgaagcaccactggctgtacaccagcaagtacctgcagaaatt
catgaacgcccacaacgccctcaagaccagcggctttaccatgaatcctcaggccagcctgggcgatccttt
Illustrative nucleotide encoding a RAG1 polypeptide fragment 7
(SEQ ID NO: 23)
cccagaaacgtgaccatggaatggcaccctcacacacccagctgcgacatctgcaacacagccagaagaggcct
gaagcggaagtccctgcagcctaatctgcagctgagcaagaaactgaaaaccgtgctggaccaggccagacagg
cccggcaaagaaagagaagggcccaagccagaatcagcagcaaggacgtgatgaagaagatcgccaactgca
gcaagatccacctgagcaccaaactgctggccgtggacttccctgagcacttcgtgaagtccatcagctgccagatct
gcgagcacatcctggccgatcctgtggaaacaaactgcaagcacgtgttctgcagagtgtgcatcctgcggtgcctga
aagtgatgggcagctactgcccctcctgcagatacccttgcttccccaccgatctggaaagccctgtgaagtccttcctg
agcgtgctgaacagcctgatggtcaagtgccccgccaaagaatgcaacgaggaagtgtccctggaaaagtacaac
caccacatcagcagccacaaagagtccaaagaaatcttcgtgcacatcaacaaaggcggcagaccccggcagc
atctgctgtctcttacaagacgggcccagaagcaccggctgagagaactgaagctgcaagtgaaggcctttgccgac
aaagaggaaggcggcgacgtcaagagcgtgtgcatgaccctgtttctgctggccctgagagcccggaatgagcata
gacaggccgatgagctggaagccatcatgcaaggcaaaggcagcggactgcagcctgctgtgtgtctggctatcag
agtgaacaccttcctgtcctgcagccagtaccacaagatgtaccggaccgtgaaggccattaccggcagacagatct
tccagcctctgcacgccctgagaaacgccgagaaagttctgctgcctggctaccaccacttcgagtggcagcctcca
ctgaagaacgtgtccagcagcaccgacgtgggcatcatcgatggactgagcggactgtctagcagcgtggacgact
accccgtggacacaatcgccaagcggttcagatacgacagcgccctggtgtctgccctgatggacatggaagagga
catcctggaaggcatgcggagccaggacctggacgattacctgaacggccctttcaccgtggtggtcaaagaaagct
gtgacggcatgggcgacgtgtccgagaaacacggatctggacctgtggtgccagagaaggccgtgcggttcagctt
caccatcatgaagatcactatcgcccacagcagccagaacgtgaaagtgttcgaggaagccaagcctaacagcga
gctgtgctgcaagcctctgtgtctgatgctggccgacgagagcgatcacgagacactgaccgccattctgagccctct
gatcgccgaacgggaagccatgaagtcctccgagctgatgctcgaactcggcggcatcctgagaaccttcaagttca
tcttccgcggcaccggctacgacgagaagctcgttagagaggtggaaggcctggaagcctctggcagcgtgtacatc
tgcaccctgtgtgacgccaccagactggaagctagccagaacctggtgttccacagcatcaccagaagccacgccg
aaaacctggaaagatacgaagtgtggcggagcaacccctaccacgagagcgtggaagaactgcgggatagagt
gaagggcgtgtccgccaagcctttcatcgagacagtgcctagcatcgacgccctgcactgcgatattggcaacgccg
ccgaattctacaagatctttcagctggaaatcggcgaggtctacaagaaccccaacgcctctaaagaggaacggaa
gcgctggcaggccacactggataagcacctgagaaagaagatgaatctgaagcccatcatgaggatgaacggca
acttcgcccggaagctgatgaccaaagaaaccgtggatgccgtgtgcgagctgatcccctctgaggaaagacacga
ggccctgcgggaactgatggacctgtacctgaagatgaagcccgtgtggcggtctagctgtcctgccaaagagtgcc
ctgagtctctgtgccagtacagcttcaacagccagagattcgccgagctgctgtccaccaagttcaagtacagatacg
agggcaagatcaccaactacttccacaagaccctggctcacgtgcccgagatcatcgagagagatggctctattggc
gcctgggcctctgagggcaatgagtctggcaacaagctgttccggcggttccgcaagatgaacgccagacagagca
agtgctacgagatggaagatgtgctgaagcaccactggctgtacaccagcaagtacctgcagaaattcatgaacgc
ccacaacgccctcaagaccagcggctttaccatgaatcctcaggccagcctgggcgatccttt
Illustrative nucleotide encoding a RAG1 polypeptide fragment 8
(SEQ ID NO: 24)
ccctagaaaagtacaaccaccacatcagcagccacaaagagtccaaagaaatcttcgtgcacatcaacaaaggc
ggcagaccccggcagcatctgctgtctcttacaagacgggcccagaagcaccggctgagagaactgaagctgcaa
gtgaaggcctttgccgacaaagaggaaggcggcgacgtcaagagcgtgtgcatgaccctgtttctgctggccctgag
agcccggaatgagcatagacaggccgatgagctggaagccatcatgcaaggcaaaggcagcggactgcagcct
gctgtgtgtctggctatcagagtgaacaccttcctgtcctgcagccagtaccacaagatgtaccggaccgtgaaggcc
attaccggcagacagatcttccagcctctgcacgccctgagaaacgccgagaaagttctgctgcctggctaccacca
cttcgagtggcagcctccactgaagaacgtgtccagcagcaccgacgtgggcatcatcgatggactgagcggactgt
ctagcagcgtggacgactaccccgtggacacaatcgccaagcggttcagatacgacagcgccctggtgtctgccctg
atggacatggaagaggacatcctggaaggcatgcggagccaggacctggacgattacctgaacggccctttcacc
gtggtggtcaaagaaagctgtgacggcatgggcgacgtgtccgagaaacacggatctggacctgtggtgccagag
aaggccgtgcggttcagcttcaccatcatgaagatcactatcgcccacagcagccagaacgtgaaagtgttcgagga
agccaagcctaacagcgagctgtgctgcaagcctctgtgtctgatgctggccgacgagagcgatcacgagacactg
accgccattctgagccctctgatcgccgaacgggaagccatgaagtcctccgagctgatgctcgaactcggcggcat
cctgagaaccttcaagttcatcttccgcggcaccggctacgacgagaagctcgttagagaggtggaaggcctggaa
gcctctggcagcgtgtacatctgcaccctgtgtgacgccaccagactggaagctagccagaacctggtgttccacagc
atcaccagaagccacgccgaaaacctggaaagatacgaagtgtggcggagcaacccctaccacgagagcgtgg
aagaactgcgggatagagtgaagggcgtgtccgccaagcctttcatcgagacagtgcctagcatcgacgccctgca
ctgcgatattggcaacgccgccgaattctacaagatctttcagctggaaatcggcgaggtctacaagaaccccaacg
cctctaaagaggaacggaagcgctggcaggccacactggataagcacctgagaaagaagatgaatctgaagccc
atcatgaggatgaacggcaacttcgcccggaagctgatgaccaaagaaaccgtggatgccgtgtgcgagctgatcc
cctctgaggaaagacacgaggccctgcgggaactgatggacctgtacctgaagatgaagcccgtgtggcggtctag
ctgtcctgccaaagagtgccctgagtctctgtgccagtacagcttcaacagccagagattcgccgagctgctgtccacc
aagttcaagtacagatacgagggcaagatcaccaactacttccacaagaccctggctcacgtgcccgagatcatcg
agagagatggctctattggcgcctgggcctctgagggcaatgagtctggcaacaagctgttccggcggttccgcaag
atgaacgccagacagagcaagtgctacgagatggaagatgtgctgaagcaccactggctgtacaccagcaagtac
ctgcagaaattcatgaacgcccacaacgccctcaagaccagcggctttaccatgaatcctcaggccagcctgggcg
atccttt
Illustrative nucleotide encoding a RAG1 polypeptide fragment 9
(SEQ ID NO: 158)
cccagaaacgtgaccatggaatggcaccctcacacacccagctgcgacatctgcaacacagccagaagaggcct
gaagcggaagtccctgcagcctaatctgcagctgagcaagaaactgaaaaccgtgctggaccaggccagacagg
cccggcaaagaaagagaagggcccaagccagaatcagcagcaaggacgtgatgaagaagatcgccaactgca
gcaagatccacctgagcaccaaactgctggccgtggacttccctgagcacttcgtgaagtccatcagctgccagatct
gcgagcacatcctggccgatcctgtggaaacaaactgcaagcacgtgttctgcagagtgtgcatcctgcggtgcctga
aagtgatgggcagctactgcccctcctgcagatacccttgcttccccaccgatctggaaagccctgtgaagtccttcctg
agcgtgctgaacagcctgatggtcaagtgccccgccaaagaatgcaacgaggaagtgtccctggaaaagtacaac
caccacatcagcagccacaaagagtccaaagaaatcttcgtgcacatcaacaaaggcggcagaccccggcagc
atctgctgtctcttacaagacgggcccagaagcaccggctgagagaactgaagctgcaagtgaaggcctttgccgac
aaagaggaaggcggcgacgtcaagagcgtgtgcatgaccctgtttctgctggccctgagagcccggaatgagcata
gacaggccgatgagctggaagccatcatgcaaggcaaaggcagcggactgcagcctgctgtgtgtctggctatcag
agtgaacaccttcctgtcctgcagccagtaccacaagatgtaccggaccgtgaaggccattaccggcagacagatct
tccagcctctgcacgccctgagaaacgccgagaaagttctgctgcctggctaccaccacttcgagtggcagcctcca
ctgaagaacgtgtccagcagcaccgacgtgggcatcatcgatggactgagcggactgtctagcagcgtggacgact
accccgtggacacaatcgccaagcggttcagatacgacagcgccctggtgtctgccctgatggacatggaagagga
catcctggaaggcatgcggagccaggacctggacgattacctgaacggccctttcaccgtggtggtcaaagaaagct
gtgacggcatgggcgacgtgtccgagaaacacggatctggacctgtggtgccagagaaggccgtgcggttcagctt
caccatcatgaagatcactatcgcccacagcagccagaacgtgaaagtgttcgaggaagccaagcctaacagcga
gctgtgctgcaagcctctgtgtctgatgctggccgacgagagcgatcacgagacactgaccgccattctgagccctct
gatcgccgaacgggaagccatgaagtcctccgagctgatgctcgaactcggcggcatcctgagaaccttcaagttca
tcttccgcggcaccggctacgacgagaagctcgttagagaggtggaaggcctggaagcctctggcagcgtgtacatc
tgcaccctgtgtgacgccaccagactggaagctagccagaacctggtgttccacagcatcaccagaagccacgccg
aaaacctggaaagatacgaagtgtggcggagcaacccctaccacgagagcgtggaagaactgcgggatagagt
gaagggcgtgtccgccaagcctttcatcgagacagtgcctagcatcgacgccctgcactgcgatattggcaacgccg
ccgaattctacaagatctttcagctggaaatcggcgaggtctacaagaaccccaacgcctctaaagaggaacggaa
gcgctggcaggccacactggataagcacctgagaaagaagatgaatctgaagcccatcatgaggatgaacggca
acttcgcccggaagctgatgaccaaagaaaccgtggatgccgtgtgcgagctgatcccctctgaggaaagacacga
ggccctgcgggaactgatggacctgtacctgaagatgaagcccgtgtggcggtctagctgtcctgccaaagagtgcc
ctgagtctctgtgccagtacagcttcaacagccagagattcgccgagctgctgtccaccaagttcaagtacagatacg
agggcaagatcaccaactacttccacaagaccctggctcacgtgcccgagatcatcgagagagatggctctattggc
gcctgggcctctgagggcaatgagtctggcaacaagctgttccggcggttccgcaagatgaacgccagacagagca
agtgctacgagatggaagatgtgctgaagcaccactggctgtacaccagcaagtacctgcagaaattcatgaacgc
ccacaacgccctcaagaccagcggctttaccatgaatcctcaggccagcctgggcgatcctctgggaattgaggata
gcctggaatcccaggacagcatggaattctga
Illustrative nucleotide encoding a RAG1 polypeptide fragment 10
(SEQ ID NO: 159)
gcgaagtgtacttccccagaaacgtgaccatggaatggcaccctcacacacccagctgcgacatctgcaacacagc
cagaagaggcctgaagcggaagtccctgcagcctaatctgcagctgagcaagaaactgaaaaccgtgctggacc
aggccagacaggcccggcaaagaaagagaagggcccaagccagaatcagcagcaaggacgtgatgaagaag
atcgccaactgcagcaagatccacctgagcaccaaactgctggccgtggacttccctgagcacttcgtgaagtccatc
agctgccagatctgcgagcacatcctggccgatcctgtggaaacaaactgcaagcacgtgttctgcagagtgtgcatc
ctgcggtgcctgaaagtgatgggcagctactgcccctcctgcagatacccttgcttccccaccgatctggaaagccctg
tgaagtccttcctgagcgtgctgaacagcctgatggtcaagtgccccgccaaagaatgcaacgaggaagtgtccctg
gaaaagtacaaccaccacatcagcagccacaaagagtccaaagaaatcttcgtgcacatcaacaaaggcggcag
accccggcagcatctgctgtctcttacaagacgggcccagaagcaccggctgagagaactgaagctgcaagtgaa
ggcctttgccgacaaagaggaaggcggcgacgtcaagagcgtgtgcatgaccctgtttctgctggccctgagagccc
ggaatgagcatagacaggccgatgagctggaagccatcatgcaaggcaaaggcagcggactgcagcctgctgtgt
gtctggctatcagagtgaacaccttcctgtcctgcagccagtaccacaagatgtaccggaccgtgaaggccattaccg
gcagacagatcttccagcctctgcacgccctgagaaacgccgagaaagttctgctgcctggctaccaccacttcgagt
ggcagcctccactgaagaacgtgtccagcagcaccgacgtgggcatcatcgatggactgagcggactgtctagcag
cgtggacgactaccccgtggacacaatcgccaagcggttcagatacgacagcgccctggtgtctgccctgatggac
atggaagaggacatcctggaaggcatgcggagccaggacctggacgattacctgaacggccctttcaccgtggtgg
tcaaagaaagctgtgacggcatgggcgacgtgtccgagaaacacggatctggacctgtggtgccagagaaggccg
tgcggttcagcttcaccatcatgaagatcactatcgcccacagcagccagaacgtgaaagtgttcgaggaagccaag
cctaacagcgagctgtgctgcaagcctctgtgtctgatgctggccgacgagagcgatcacgagacactgaccgccat
tctgagccctctgatcgccgaacgggaagccatgaagtcctccgagctgatgctcgaactcggcggcatcctgagaa
ccttcaagttcatcttccgcggcaccggctacgacgagaagctcgttagagaggtggaaggcctggaagcctctggc
agcgtgtacatctgcaccctgtgtgacgccaccagactggaagctagccagaacctggtgttccacagcatcaccag
aagccacgccgaaaacctggaaagatacgaagtgtggcggagcaacccctaccacgagagcgtggaagaactg
cgggatagagtgaagggcgtgtccgccaagcctttcatcgagacagtgcctagcatcgacgccctgcactgcgatatt
ggcaacgccgccgaattctacaagatctttcagctggaaatcggcgaggtctacaagaaccccaacgcctctaaag
aggaacggaagcgctggcaggccacactggataagcacctgagaaagaagatgaatctgaagcccatcatgagg
atgaacggcaacttcgcccggaagctgatgaccaaagaaaccgtggatgccgtgtgcgagctgatcccctctgagg
aaagacacgaggccctgcgggaactgatggacctgtacctgaagatgaagcccgtgtggcggtctagctgtcctgcc
aaagagtgccctgagtctctgtgccagtacagcttcaacagccagagattcgccgagctgctgtccaccaagttcaag
tacagatacgagggcaagatcaccaactacttccacaagaccctggctcacgtgcccgagatcatcgagagagatg
gctctattggcgcctgggcctctgagggcaatgagtctggcaacaagctgttccggcggttccgcaagatgaacgcca
gacagagcaagtgctacgagatggaagatgtgctgaagcaccactggctgtacaccagcaagtacctgcagaaatt
catgaacgcccacaacgccctcaagaccagcggctttaccatgaatcctcaggccagcctgggcgatcctctggga
attgaggatagcctggaatcccaggacagcatggaattctga

### Polynucleotides and Genomes

In one aspect, the present invention provides a polynucleotide comprising from 5′ to 3′: a first homology region, a nucleotide sequence encoding a RAG1 polypeptide or a RAG1 polypeptide fragment, and a second homology region. The first homology region may be homologous to a first region of the RAG1 intron 1 or exon 2 and the second homology region may be homologous to a second region of the RAG1 exon 2. The polynucleotide may be an isolated polynucleotide. The polynucleotide may be a DNA molecule, e.g. a double-stranded DNA molecule.

Suitably, the polynucleotide of the invention may be limited to a size suitable to be inserted into a vector (e.g. an adeno-associated viral (AAV) vector, such as AAV6). Suitably, the polynucleotide of the invention may be 5.0 kb or less, 4.9 kb or less, 4.8 kb or less, 4.7 kb or less, 4.6 kb or less, 4.5 kb or less, 4.4 kb or less, 4.3 kb or less, 4.2 kb or less, 4.1 kb or less, 4.0 kb or less in total size. In some embodiments, the polynucleotide of the invention is 4.1 kb or less or 4.0 kb or less in size.

In another aspect, the present invention provides a genome comprising a nucleotide sequence encoding a RAG1 polypeptide or a RAG1 polypeptide fragment. Suitably, the genome may comprise the polynucleotide of the present invention. The genome may be an isolated genome. The genome may be a mammalian genome, e.g. a human genome.

### Homology Regions

A “homology region” (also known as “homology arm”) is a nucleotide sequence which is located upstream or downstream of a nucleotide sequence to be inserted (a “nucleotide sequence insert” e.g. a splice acceptor sequence and a nucleotide sequence encoding a RAG1 polypeptide). The polynucleotide of the present invention comprises two homology regions, one upstream of the nucleotide sequence insert (the “first homology region”) and one downstream of the nucleotide insert (the “second homology region”).

Each “homology region” is designed such that the nucleotide sequence insert can be introduced into a genome at a site of a double strand break (DSB) by homology-directed repair (HDR). One of skill in the art will be able to design homology arms depending on the desired insertion site (i.e. the site of the DSB) (see e.g. Ran, F. A., et al., 2013. Nature protocols, 8(11), pp. 2281-2308). Each “homology region” is homologous to a region either side of the DSB. For example, the first homology region may be homologous to a region upstream of the DSB and the second homology region may be homologous to a region downstream of the DSB.

As used herein, the term “homologous” means that the nucleotide sequences are similar or identical. For example, the nucleotide sequences may be at least 70% identical, at least 75% identical, at least 80% identical, at least 85% identical, at least 90% identical, at least 95% identical, at least 98% identical, at least 99% identical, or 100% identical.

As used herein, “upstream” and “downstream” both refer to relative positions in DNA or RNA. Each strand of DNA or RNA has a 5′ end and a 3′ end and, by convention, “upstream” and “downstream” relate to the 5′ to 3′ direction respectively in which RNA transcription takes place. For example, when considering double-stranded DNA, “upstream” is toward the 5′ end of the coding strand for the gene in question (e.g. RAG1) and downstream is toward the 3′ end of the coding strand for the gene in question (e.g. RAG1).

The homology regions may be any length suitable for HDR. The homology regions may be the same or different lengths. Suitably, the homology regions are each independently 50-2000 bp in length, 50-1800 bp in length, 50-1500 bp in length, 50-1000 bp in length, 100-500 bp in length, or 200-400 bp in length. For example, the first homology region may be 50-2000 bp in length and homologous to a region upstream of a DSB and the second homology region may be 50-2000 bp in length and homologous to a region downstream of the DSB.

In some embodiments, the first homology region is about 50-1000 bp in length, 100-500 bp in length, or 200-400 bp in length and the second homology region is about 50-1000 bp in length, 100-500 bp in length, or 200-400 bp in length. In other embodiments, the first homology region is about 50-1000 bp in length, 100-500 bp in length, or 200-400 bp in length and the second homology region is about 500-2000 bp in length, 800-2000 bp in length, 1000-2000 bp in length, or 1500-2000 bp in length.

In some embodiments:
(i) the first homology region is homologous to a first region of the RAG1 exon 2 and the second homology region is homologous to a second region of the RAG1 exon 2; or
(ii) the first homology region is homologous to a first region of the RAG1 intron 1 or the start of the RAG1 exon 2 (e.g. the first 200 bp of the RAG1 exon 2) and the second homology region is homologous to a second region of the RAG1 exon 2, preferably wherein the first homology region is homologous to a region of the RAG1 intron 1 and the second homology region is homologous to a region of the RAG1 exon 2.

As used herein, embodiment (i) may be referred to as an “exon 2 RAG1 gene strategy” and embodiment (ii) may be referred to as an “intron 1 RAG1 gene strategy”.

### Exon 2 Strategies

In preferred embodiments, the first homology region is homologous to a first region of the RAG1 exon 2 and the second homology region is homologous to a second region of the RAG1 exon 2.

In some embodiments:
(i) the first homology region is homologous to a region upstream of chr 11:36574368 and the second homology region is homologous to a region downstream of chr 11:36574369;
(ii) the first homology region is homologous to a region upstream of chr 11:36574367 and the second homology region is homologous to a region downstream of chr 11:36574368;
(iii) the first homology region is homologous to a region upstream of chr 11:36574394 and the second homology region is homologous to a region downstream of chr 11:36574395;
(iv) the first homology region is homologous to a region upstream of chr 11:36574294 and the second homology region is homologous to a region downstream of chr 11:36574295;
(v) the first homology region is homologous to a region upstream of chr 11:36574109 and the second homology region is homologous to a region downstream of chr 11:36574110;
(vi) the first homology region is homologous to a region upstream of chr 11:36573910 and the second homology region is homologous to a region downstream of chr 11:36573911;
(vii) the first homology region is homologous to a region upstream of chr 11:36573878 and the second homology region is homologous to a region downstream of chr 11:36573879;
(viii) the first homology region is homologous to a region upstream of chr 11:36573959 and the second homology region is homologous to a region downstream of chr 11:36573960;
(ix) the first homology region is homologous to a region upstream of chr 11:36573957 and the second homology region is homologous to a region downstream of chr 11:36573958;
(x) the first homology region is homologous to a region upstream of chr 11:36573879 and the second homology region is homologous to a region downstream of chr 11:36573880;
(xi) the first homology region is homologous to a region upstream of chr 11:36573892 and the second homology region is homologous to a region downstream of chr 11:36573893;
(xii) the first homology region is homologous to a region upstream of chr 11:36573955 and the second homology region is homologous to a region downstream of chr 11:36573956;
(xiii) the first homology region is homologous to a region upstream of chr 11:36573878 and the second homology region is homologous to a region downstream of chr 11:36573879; or
(xiv) the first homology region is homologous to a region upstream of chr 11:36574406 and the second homology region is homologous to a region downstream of chr 11:36574407.

In some embodiments:
(v) the first homology region is homologous to a region upstream of chr 11:36574109 and the second homology region is homologous to a region downstream of chr 11:36574110; or
(vi) the first homology region is homologous to a region upstream of chr 11:36573910 and the second homology region is homologous to a region downstream of chr 11:36573911.

In some embodiments:
(xi) the first homology region is homologous to a region upstream of chr 11:36573892 and the second homology region is homologous to a region downstream of chr 11:36573893; or
(xiii) the first homology region is homologous to a region upstream of chr 11:36573878 and the second homology region is homologous to a region downstream of chr 11:36573879.

In some embodiments, the first homology region is homologous to a region upstream of chr 11:36573878 and the second homology region is homologous to a region downstream of chr 11:36573879.

The first homology region may be homologous to a region immediately upstream of the DSB.

In some embodiments, the second homology region is: (a) homologous to a region immediately downstream of the DSB; or (b) homologous to a region distantly downstream of the DSB.

As used herein, embodiment (a) may be referred to as an “exon 2 RAG1 gene targeting strategy” and embodiment (b) may be referred to as an “exon 2 RAG1 gene replacement strategy”.

As used herein, “immediately upstream” or “immediately downstream” may mean the region is 100 bp or less, 50 bp or less, 40 bp or less, 30 bp or less, 20 bp or less, 10 bp or less, 5 bp or less, 4 bp or less, 3 bp or less, 2 bp or less, or 1 bp upstream of the DSB.

As used herein, “distantly downstream” may mean the region is 150 bp or more, 200 bp or more, 250 bp or more, 300 bp or more, 350 bp or more, 400 bp or more, 450 bp or more, 500 bp or more, 600 bp or more, 700 bp or more, 800 bp or more, 900 bp or more, 1000 bp or more, 1500 bp or more, or 2000 bp or more downstream of the DSB. For example, a distantly downstream region may be downstream of chr 11:36574557; downstream of chr 11: 36574870; downstream of chr 11:36575183; downstream of chr 11:36575496; downstream of chr 11:36575810; downstream of chr 11:36576123; or downstream of chr 11:36576436

In some embodiments:
(i) the first homology region is homologous to a region comprising chr 11:36574319-36574368 and/or the second homology region is homologous to: (a) a region comprising chr 11:36574369-36574418; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536;
(ii) the first homology region is homologous to a region comprising chr 11:36574318-36574367 and/or the second homology region is homologous to: (a) a region comprising chr 11:36574368-36574417; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536;
(iii) the first homology region is homologous to a region comprising chr 11:36574345-36574394 and/or the second homology region is homologous to: (a) a region comprising chr 11:36574395-36574444; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536;
(iv) the first homology region is homologous to a region comprising chr 11:36574245-36574294 and/or the second homology region is homologous to: (a) a region comprising chr 11:36574295-36574344; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536;
(v) the first homology region is homologous to a region comprising chr 11:36574060-36574109 and/or the second homology region is homologous to: (a) a region comprising chr 11:36574110-36574159; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536;
(vi) the first homology region is homologous to a region comprising chr 11:36573861-36573910 and/or the second homology region is homologous to: (a) a region comprising chr 11:36573911-36573960; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536;
(vii) the first homology region is homologous to a region comprising chr 11:36573829-36573878 and/or the second homology region is homologous to: (a) a region comprising chr 11:36573879-36573928; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536;
(viii) the first homology region is homologous to a region comprising chr 11:36573910-36573959 and/or the second homology region is homologous to: (a) a region comprising chr 11:36573960-36574009; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536;
(ix) the first homology region is homologous to a region comprising chr 11:36573908-36573957 and/or the second homology region is homologous to: (a) a region comprising chr 11:36573958-36574007; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536;
(x) the first homology region is homologous to a region comprising chr 11:36573830-36573879 and/or the second homology region is homologous to: (a) a region comprising chr 11:36573880-36573929; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536;
(xi) the first homology region is homologous to a region comprising chr 11:36573843-36573892 and/or the second homology region is homologous to: (a) a region comprising chr 11:36573893-36573942; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536;
(xii) the first homology region is homologous to a region comprising chr 11:36573906-36573955 and/or the second homology region is homologous to: (a) a region comprising chr 11:36573956-36574005; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536;
(xiii) the first homology region is homologous to a region comprising chr 11:36573829-36573878 and/or the second homology region is homologous to: (a) a region comprising chr 11:36573879-36573928; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536; or
(xiv) the first homology region is homologous to a region comprising chr 11:36574357-36574406 and/or the second homology region is homologous to: (a) a region comprising chr 11:36574407-36574456; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536.

In some embodiments:
(v) the first homology region is homologous to a region comprising chr 11:36574060-36574109 and/or the second homology region is homologous to: (a) a region comprising chr 11:36574110-36574159; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536; or
(vi) the first homology region is homologous to a region comprising chr 11:36573861-36573910 and/or the second homology region is homologous to: (a) a region comprising chr 11:36573911-36573960; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536.

In some embodiments:
(xi) the first homology region is homologous to a region comprising chr 11:36573843-36573892 and/or the second homology region is homologous to: (a) a region comprising chr 11:36573893-36573942; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536; or
(xiii) the first homology region is homologous to a region comprising chr 11:36573829-36573878 and/or the second homology region is homologous to: (a) a region comprising chr 11:36573879-36573928; or (b) a region comprising chr 11:36574558-36574657, a region comprising chr 11:36574871-36574970, a region comprising chr 11:36575184-36575283, a region comprising chr 11:36575497-36575596, a region comprising chr 11:36575811-36575910, a region comprising chr 11:36576124-36576223, or a region comprising chr 11:36576437-36576536.

In some embodiments:
(i) the first homology region is homologous to a region comprising chr 11:36574319-36574368 and/or the second homology region is homologous to: (a) a region comprising chr 11:36574369-36574418; or (b) a region comprising chr 11:36576437-36576536;
(ii) the first homology region is homologous to a region comprising chr 11:36574318-36574367 and/or the second homology region is homologous to: (a) a region comprising chr 11:36574368-36574417; or (b) a region comprising chr 11:36576437-36576536;
(iii) the first homology region is homologous to a region comprising chr 11:36574345-36574394 and/or the second homology region is homologous to: (a) a region comprising chr 11:36574395-36574444; or (b) a region comprising chr 11:36576437-36576536;
(iv) the first homology region is homologous to a region comprising chr 11:36574245-36574294 and/or the second homology region is homologous to: (a) a region comprising chr 11:36574295-36574344; or (b) a region comprising chr 11:36576437-36576536;
(v) the first homology region is homologous to a region comprising chr 11:36574060-36574109 and/or the second homology region is homologous to: (a) a region comprising chr 11:36574110-36574159; or (b) a region comprising chr 11:36576437-36576536;
(vi) the first homology region is homologous to a region comprising chr 11:36573861-36573910 and/or the second homology region is homologous to: (a) a region comprising chr 11:36573911-36573960; or (b) a region comprising chr 11:36576437-36576536;
(vii) the first homology region is homologous to a region comprising chr 11:36573829-36573878 and/or the second homology region is homologous to: (a) a region comprising chr 11:36573879-36573928; or (b) a region comprising chr 11:36576437-36576536;
(viii) the first homology region is homologous to a region comprising chr 11:36573910-36573959 and/or the second homology region is homologous to: (a) a region comprising chr 11:36573960-36574009; or (b) a region comprising chr 11:36576437-36576536;
(ix) the first homology region is homologous to a region comprising chr 11:36573908-36573957 and/or the second homology region is homologous to: (a) a region comprising chr 11:36573958-36574007; or (b) a region comprising chr 11:36576437-36576536;
(x) the first homology region is homologous to a region comprising chr 11:36573830-36573879 and/or the second homology region is homologous to: (a) a region comprising chr 11:36573880-36573929; or (b) a region comprising chr 11:36576437-36576536;
(xi) the first homology region is homologous to a region comprising chr 11:36573843-36573892 and/or the second homology region is homologous to: (a) a region comprising chr 11:36573893-36573942; or (b) a region comprising chr 11:36576437-36576536;
(xii) the first homology region is homologous to a region comprising chr 11:36573906-36573955 and/or the second homology region is homologous to: (a) a region comprising chr 11:36573956-36574005; or (b) a region comprising chr 11:36576437-36576536;
(xiii) the first homology region is homologous to a region comprising chr 11:36573829-36573878 and/or the second homology region is homologous to: (a) a region comprising chr 11:36573879-36573928; or (b) a region comprising chr 11:36576437-36576536; or
(xiv) the first homology region is homologous to a region comprising chr 11:36574357-36574406 and/or the second homology region is homologous to: (a) a region comprising chr 11:36574407-36574456; or (b) a region comprising chr 11:36576437-36576536.

In some embodiments:
(v) the first homology region is homologous to a region comprising chr 11:36574060-36574109 and/or the second homology region is homologous to: (a) a region comprising chr 11:36574110-36574159; or (b) a region comprising chr 11:36576437-36576536; or
(vi) the first homology region is homologous to a region comprising chr 11:36573861-36573910 and/or the second homology region is homologous to: (a) a region comprising chr 11:36573911-36573960; or (b) a region comprising chr 11:36576437-36576536.

In some embodiments:
(xi) the first homology region is homologous to a region comprising chr 11:36573843-36573892 and/or the second homology region is homologous to: (a) a region comprising chr 11:36573893-36573942; or (b) a region comprising chr 11:36576437-36576536; or
(xiii) the first homology region is homologous to a region comprising chr 11:36573829-36573878 and/or the second homology region is homologous to: (a) a region comprising chr 11:36573879-36573928; or (b) a region comprising chr 11:36576437-36576536.

In some embodiments, the first homology region is homologous to a region comprising chr 11:36573829-36573878 and/or the second homology region is homologous to: (a) a region comprising chr 11:36573879-36573928; or (b) a region comprising chr 11:36576437-36576536.

In some embodiments:
(i) the first homology region is homologous to a region comprising chr 11:36574319-36574368 and/or the second homology region is homologous to a region comprising chr 11:36574369-36574418;
(ii) the first homology region is homologous to a region comprising chr 11:36574318-36574367 and/or the second homology region is homologous to a region comprising chr 11:36574368-36574417;
(iii) the first homology region is homologous to a region comprising chr 11:36574345-36574394 and/or the second homology region is homologous to a region comprising chr 11:36574395-36574444;
(iv) the first homology region is homologous to a region comprising chr 11:36574245-36574294 and/or the second homology region is homologous to a region comprising chr 11:36574295-36574344;
(v) the first homology region is homologous to a region comprising chr 11:36574060-36574109 and/or the second homology region is homologous to a region comprising chr 11:36574110-36574159;
(vi) the first homology region is homologous to a region comprising chr 11:36573861-36573910 and/or the second homology region is homologous to a region comprising chr 11:36573911-36573960;
(vii) the first homology region is homologous to a region comprising chr 11:36573829-36573878 and/or the second homology region is homologous to a region comprising chr 11:36573879-36573928;
(viii) the first homology region is homologous to a region comprising chr 11:36573910-36573959 and/or the second homology region is homologous to a region comprising chr 11:36573960-36574009;
(ix) the first homology region is homologous to a region comprising chr 11:36573908-36573957 and/or the second homology region is homologous to a region comprising chr 11:36573958-36574007;
(x) the first homology region is homologous to a region comprising chr 11:36573830-36573879 and/or the second homology region is homologous to a region comprising chr 11:36573880-36573929;
(xi) the first homology region is homologous to a region comprising chr 11:36573843-36573892 and/or the second homology region is homologous to a region comprising chr 11:36573893-36573942;
(xii) the first homology region is homologous to a region comprising chr 11:36573906-36573955 and/or the second homology region is homologous to a region comprising chr 11:36573956-36574005;
(xiii) the first homology region is homologous to a region comprising chr 11:36573829-36573878 and/or the second homology region is homologous to a region comprising chr 11:36573879-36573928; or
(xiv) the first homology region is homologous to a region comprising chr 11:36574357-36574406 and/or the second homology region is homologous to a region comprising chr 11:36574407-36574456.

In some embodiments:
(v) the first homology region is homologous to a region comprising chr 11:36574060-36574109 and/or the second homology region is homologous to a region comprising chr 11:36574110-36574159; or
(vi) the first homology region is homologous to a region comprising chr 11:36573861-36573910 and/or the second homology region is homologous to a region comprising chr 11:36573911-36573960.

In some embodiments:
(xi) the first homology region is homologous to a region comprising chr 11:36573843-36573892 and/or the second homology region is homologous to a region comprising chr 11:36573893-36573942; or
(xiii) the first homology region is homologous to a region comprising chr 11:36573829-36573878 and/or the second homology region is homologous to a region comprising chr 11:36573879-36573928.

In some embodiments, the first homology region is homologous to a region comprising chr 11:36573829-36573878 and/or the second homology region is homologous to a region comprising chr 11:36573879-36573928.

In some embodiments:
(i) the first homology region is homologous to a region comprising chr 11:36574319-36574368 and/or the second homology region is homologous to a region comprising chr 11:36576437-36576536;
(ii) the first homology region is homologous to a region comprising chr 11:36574318-36574367 and/or the second homology region is homologous to a region comprising chr 11:36576437-36576536;
(iii) the first homology region is homologous to a region comprising chr 11:36574345-36574394 and/or the second homology region is homologous to a region comprising chr 11:36576437-36576536;
(iv) the first homology region is homologous to a region comprising chr 11:36574245-36574294 and/or the second homology region is homologous to a region comprising chr 11:36576437-36576536;
(v) the first homology region is homologous to a region comprising chr 11:36574060-36574109 and/or the second homology region is homologous to a region comprising chr 11:36576437-36576536;
(vi) the first homology region is homologous to a region comprising chr 11:36573861-36573910 and/or the second homology region is homologous to a region comprising chr 11:36576437-36576536;
(vii) the first homology region is homologous to a region comprising chr 11:36573829-36573878 and/or the second homology region is homologous to a region comprising chr 11:36576437-36576536;
(viii) the first homology region is homologous to a region comprising chr 11:36573910-36573959 and/or the second homology region is homologous to a region comprising chr 11:36576437-36576536;
(ix) the first homology region is homologous to a region comprising chr 11:36573908-36573957 and/or the second homology region is homologous to a region comprising chr 11:36576437-36576536;
(x) the first homology region is homologous to a region comprising chr 11:36573830-36573879 and/or the second homology region is homologous to a region comprising chr 11:36576437-36576536;
(xi) the first homology region is homologous to a region comprising chr 11:36573843-36573892 and/or the second homology region is homologous to a region comprising chr 11:36576437-36576536;
(xii) the first homology region is homologous to a region comprising chr 11:36573906-36573955 and/or the second homology region is homologous to a region comprising chr 11:36576437-36576536;
(xiii) the first homology region is homologous to a region comprising chr 11:36573829-36573878 and/or the second homology region is homologous to a region comprising chr 11:36576437-36576536; or
(xiv) the first homology region is homologous to a region comprising chr 11:36574357-36574406 and/or the second homology region is homologous to a region comprising chr 11:36576437-36576536.

In some embodiments:
(v) the first homology region is homologous to a region comprising chr 11:36574060-36574109 and/or the second homology region is homologous to a region comprising chr 11:36576437-36576536; or
(vi) the first homology region is homologous to a region comprising chr 11:36573861-36573910 and/or the second homology region is homologous to a region comprising chr 11:36576437-36576536.

In some embodiments:
(xi) the first homology region is homologous to a region comprising chr 11:36573843-36573892 and/or the second homology region is homologous to a region comprising chr 11:36576437-36576536; or
(xiii) the first homology region is homologous to a region comprising chr 11:36573829-36573878 and/or the second homology region is homologous to a region comprising chr 11:36576437-36576536.

In some embodiments, the first homology region is homologous to a region comprising chr 11:36573829-36573878 and/or the second homology region is homologous to a region comprising chr 11:36576437-36576536.

Exemplary first homology regions for the exon 2 strategies are shown below in Tables 1 and 2.

In some embodiments, the first homology region comprises or consists of a nucleotide sequence that has at least 70% identity, at least 80% identity, at least 90% identity, at least 95% identity, at least 98% identity, or 100% identity to any of SEQ ID NOs: 25-44.

TABLE 1
Exemplary first homology regions for exon 2 strategies

| Guide RNA | First homology region |
|---|---|
| g1 M5 ex2 | gacctggagagtccagtgaagtcctttctgagcgtcttgaattccctgat (SEQ ID NO: 25) |
| g2 M5 ex2 | tgacctggagagtccagtgaagtcctttctgagcgtcttgaattccctga (SEQ ID NO: 26) |
| g3 M5 ex2 | tctgagcgtcttgaattccctgatggtgaaatgtccagcaaaagagtgca (SEQ ID NO: 27) |
| g4 M4 ex2 | gggtctgcattctcagatgcctcaaagtcatgggcagctattgtccctct (SEQ ID NO: 28) |
| g5 M3 ex2 | agctcaggcaaggatcagcagcaaggatgtcatgaagaagatcgccaact (SEQ ID NO: 29) |
| g6 M2 ex2 | agtttagcagtgccccatgtgaggtttacttcccgaggaacgtgaccatg (SEQ ID NO: 30) |
| g7 exon2 M2/3 | ctgccataactgctggagcatcatgcacaggaagtttagcagtgccccat (SEQ ID NO: 31) |
| g8 exon2 M2/3 | ggagtggcacccccacacaccatcctgtgacatctgcaacactgcccgtc (SEQ ID NO: 32) |
| g9 exon2 M2/3 | atggagtggcacccccacacaccatcctgtgacatctgcaacactgcccg (SEQ ID NO: 33) |
| g10 exon2 M2/3 | tgccataactgctggagcatcatgcacaggaagtttagcagtgccccatg (SEQ ID NO: 34) |
| g11 exon2 M2/3 | ggagcatcatgcacaggaagtttagcagtgccccatgtgaggtttacttc (SEQ ID NO: 35) |
| g12 exon2 M2/3 | ccatggagtggcacccccacacaccatcctgtgacatctgcaacactgcc (SEQ ID NO: 36) |
| g13 exon2 M2/3 | ctgccataactgctggagcatcatgcacaggaagtttagcagtgccccat (SEQ ID NO: 37) |
| g14 exon2 M5 | gaattccctgatggtgaaatgtccagcaaaagagtgcaatgaggaggtca (SEQ ID NO: 38) |

TABLE 2
Exemplary first homology regions for exon 2 strategies

| Guide RNA | First homology region |
|---|---|
| g5 M3 ex2 | AGCTCAGGCAAGGATCAGCAGCAAGGATGTCATGAAGAAGAT |
| | CGCAAACT (SEQ ID NO: 39) |
| g6 M2 ex2 | AGTTTAGCAGTGCCCCATGTGAGGTTTACTTCCCGAGGAATGT |
| | CACTATG (SEQ ID NO: 40) |
| g7 exon2 M2/3 | CTGCCATAACTGCTGGAGCATCATGCACAGGAAGTTTAGCAGT |
| g10 exon2 M2/3 | GCACCAT (SEQ ID NO: 41) |
| g13 exon2 M2/3 | |
| g8 exon2 M2/3 | ACCATGGAGTGGCACCCCCACACACCATCCTGTGACATCTGC |
| g9 exon2 M2/3 | AACACTGC (SEQ ID NO: 42) |
| g12 exon2 M2/3 | |
| g11 exon2 M2/3 | GGAGCATCATGCACAGGAAGTTTAGCAGTGCCCCATGTGAGG |
| | TTTACTTC (SEQ ID NO: 43) |
| g14 exon2 M5 | GAATTCCCTGATGGTGAAATGTCCAGCAAAAGAGTGCAATGAG |
| | GAGGTCA (SEQ ID NO: 44) |

Exemplary second homology regions for the exon 2 gene targeting strategies are shown below in Tables 3 and 4.

In some embodiments, the second homology region comprises or consists of a nucleotide sequence that has at least 70% identity, at least 80% identity, at least 90% identity, at least 95% identity, at least 98% identity, or 100% identity to any of SEQ ID NOs: 45-60.

TABLE 3
Exemplary second homology regions for exon 2 targeting strategies

| Guide RNA | Second homology region |
|---|---|
| g1 M5 ex2 | ggtgaaatgtccagcaaaagagtgcaatgaggaggtcagtttggaaaaat (SEQ ID NO: 45) |
| g2 M5 ex2 | tggtgaaatgtccagcaaaagagtgcaatgaggaggtcagtttggaaaaa (SEQ ID NO: 46) |
| g3 M5 ex2 | atgaggaggtcagtttggaaaaatataatcaccacatctcaagtcacaag (SEQ ID NO: 47) |
| g4 M4 ex2 | tgccgatatccatgcttccctactgacctggagagtccagtgaagtcctt (SEQ ID NO: 48) |
| g5 M3 ex2 | gcagtaagatacatcttagtaccaagctccttgcagtggacttcccagag (SEQ ID NO: 49) |
| g6 M2 ex2 | gagtggcacccccacacaccatcctgtgacatctgcaacactgcccgtcg (SEQ ID NO: 50) |
| g7 exon2 M2/3 | gtgaggtttacttcccgaggaacgtgaccatggagtggcacccccacaca (SEQ ID NO: 51) |
| g8 exon2 M2/3 | ggggactcaagaggaagagtcttcagccaaacttgcagctcagcaaaaaa (SEQ ID NO: 52) |
| g9 exon2 M2/3 | tcggggactcaagaggaagagtcttcagccaaacttgcagctcagcaaaa (SEQ ID NO: 53) |
| g10 exon2 M2/3 | tgaggtttacttcccgaggaacgtgaccatggagtggcacccccacacac (SEQ ID NO: 54) |
| g11 exon2 M2/3 | ccgaggaacgtgaccatggagtggcacccccacacaccatcctgtgacat (SEQ ID NO: 55) |
| g12 exon2 M2/3 | cgtcggggactcaagaggaagagtcttcagccaaacttgcagctcagcaa (SEQ ID NO: 56) |
| g13 exon2 M2/3 | gtgaggtttacttcccgaggaacgtgaccatggagtggcacccccacaca (SEQ ID NO: 57) |
| g14 exon2 M5 | gtttggaaaaatataatcaccacatctcaagtcacaaggaatcaaaagag (SEQ ID NO: 58) |

TABLE 4
Exemplary second homology regions for exon 2 targeting strategies

| Guide RNA | Second homology region |
|---|---|
| g5 M3 ex2 | gcagtaagatacatcttagtaccaagctccttgcagtggacttcccagagcactttgtgaaatccatct |
| | cctgccagatctgtgaacacattctggctga (SEQ ID NO: 59) |
| g6 M2 ex2 | gagtggcacccccacacaccatcctgtgacatctgcaacactgcccgtcggggactcaagaggaa |
| | gagtcttcagccaaacttgcagctcagcaaaaaac (SEQ ID NO: 60) |

Preferably, the first and second homology regions comprise or consist of nucleotide sequences that have at least 70% identity, at least 80% identity, at least 90% identity, at least 95% identity, at least 98% identity, or 100% identity to first and second homology regions in Tables 1 to 4, which are designed for the same guide RNAs. Suitably, the first homology region comprises or consists of a nucleotide sequence that has at least 70% identity, at least 80% identity, at least 90% identity, at least 95% identity, at least 98% identity, or 100% identity to any of SEQ ID NOs: 25-44 and the second homology region comprises or consists of a nucleotide sequence that has at least 70% identity, at least 80% identity, at least 90% identity, at least 95% identity, at least 98% identity, or 100% identity to the corresponding nucleotide sequence in Tables 3 or 4 (i.e. SEQ ID NOs: 45-60). For example, in some embodiments:
(i) the first homology region comprises or consists of a nucleotide sequence that has at least 70% identity, at least 80% identity, at least 90% identity, at least 95% identity, at least 98% identity, or 100% identity to SEQ ID NO: 25 and the second homology region comprises or consists of a nucleotide sequence that has at least 70% identity, at least 80% identity, at least 90% identity, at least 95% identity, at least 98% identity, or 100% identity to SEQ ID NO: 45;
(ii) the first homology region comprises or consists of a nucleotide sequence that has at least 70% identity, at least 80% identity, at least 90% identity, at least 95% identity, at least 98% identity, or 100% identity to SEQ ID NO: 26 and the second homology region comprises or consists of a nucleotide sequence that has at least 70% identity, at least 80% identity, at least 90% identity, at least 95% identity, at least 98% identity, or 100% identity to SEQ ID NO: 46;
(iii) the first homology region comprises or consists of a nucleotide sequence that has at least 70% identity, at least 80% identity, at least 90% identity, at least 95% identity, at least 98% identity, or 100% identity to SEQ ID NO: 27 and the second homology region comprises or consists of a nucleotide sequence that has at least 70% identity, at least 80% identity, at least 90% identity, at least 95% identity, at least 98% identity, or 100% identity to SEQ ID NO: 47;
(iv) the first homology region comprises or consists of a nucleotide sequence that has at least 70% identity, at least 80% identity, at least 90% identity, at least 95% identity, at least 98% identity, or 100% identity to SEQ ID NO: 28 and the second homology region comprises or consists of a nucleotide sequence that has at least 70% identity, at least 80% identity, at least 90% identity, at least 95% identity, at least 98% identity, or 100% identity to SEQ ID NO: 48;
(v) the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39 and the second homology region comprises or consists of a nucleotide sequence that has at least 70% identity, at least 80% identity, at least 90% identity, at least 95% identity, at least 98% identity, or 100% identity to SEQ ID NO: 49 or SEQ ID NO: 59;
(vi) the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40 and the second homology region comprises or consists of a nucleotide sequence that has at least 70% identity, at least 80% identity, at least 90% identity, at least 95% identity, at least 98% identity, or 100% identity to SEQ ID NO: 50 or SEQ ID NO: 60;
(vii) the first homology region comprises or consists of a nucleotide sequence that has at least 70% identity, at least 80% identity, at least 90% identity, at least 95% identity, at least 98% identity, or 100% identity to SEQ ID NO: 31 or SEQ ID NO: 41 and the second homology region comprises or consists of a nucleotide sequence that has at least 70% identity, at least 80% identity, at least 90% identity, at least 95% identity, at least 98% identity, or 100% identity to SEQ ID NO: 51;
(viii) the first homology region comprises or consists of a nucleotide sequence that has at least 70% identity, at least 80% identity, at least 90% identity, at least 95% identity, at least 98% identity, or 100% identity to SEQ ID NO: 32 or SEQ ID NO: 42 and the second homology region comprises or consists of a nucleotide sequence that has at least 70% identity, at least 80% identity, at least 90% identity, at least 95% identity, at least 98% identity, or 100% identity to SEQ ID NO: 52;
(ix) the first homology region comprises or consists of a nucleotide sequence that has at least 70% identity, at least 80% identity, at least 90% identity, at least 95% identity, at least 98% identity, or 100% identity to SEQ ID NO: 33 or SEQ ID NO: 42 and the second homology region comprises or consists of a nucleotide sequence that has at least 70% identity, at least 80% identity, at least 90% identity, at least 95% identity, at least 98% identity, or 100% identity to SEQ ID NO: 53;
(x) the first homology region comprises or consists of a nucleotide sequence that has at least 70% identity, at least 80% identity, at least 90% identity, at least 95% identity, at least 98% identity, or 100% identity to SEQ ID NO: 34 or SEQ ID NO: 41 and the second homology region comprises or consists of a nucleotide sequence that has at least 70% identity, at least 80% identity, at least 90% identity, at least 95% identity, at least 98% identity, or 100% identity to SEQ ID NO: 54;
(xi) the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41 and the second homology region comprises or consists of a nucleotide sequence that has at least 70% identity, at least 80% identity, at least 90% identity, at least 95% identity, at least 98% identity, or 100% identity to SEQ ID NO: 55;
(xii) the first homology region comprises or consists of a nucleotide sequence that has at least 70% identity, at least 80% identity, at least 90% identity, at least 95% identity, at least 98% identity, or 100% identity to SEQ ID NO: 36 or SEQ ID NO: 42 and the second homology region comprises or consists of a nucleotide sequence that has at least 70% identity, at least 80% identity, at least 90% identity, at least 95% identity, at least 98% identity, or 100% identity to SEQ ID NO: 56;
(xiii) the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the second homology region comprises or consists of a nucleotide sequence that has at least 70% identity, at least 80% identity, at least 90% identity, at least 95% identity, at least 98% identity, or 100% identity to SEQ ID NO: 57; or
(xiv) the first homology region comprises or consists of a nucleotide sequence that has at least 70% identity, at least 80% identity, at least 90% identity, at least 95% identity, at least 98% identity, or 100% identity to SEQ ID NO: 38 or SEQ ID NO: 44 and the second homology region comprises or consists of a nucleotide sequence that has at least 70% identity, at least 80% identity, at least 90% identity, at least 95% identity, at least 98% identity, or 100% identity to SEQ ID NO: 58.

In some embodiments:
(v) the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39 and the second homology region comprises or consists of a nucleotide sequence that has at least 70% identity, at least 80% identity, at least 90% identity, at least 95% identity, at least 98% identity, or 100% identity to SEQ ID NO: 49 or SEQ ID NO: 59; or
(vi) the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40 and the second homology region comprises or consists of a nucleotide sequence that has at least 70% identity, at least 80% identity, at least 90% identity, at least 95% identity, at least 98% identity, or 100% identity to SEQ ID NO: 50 or SEQ ID NO: 60.

In some embodiments:
(xi) the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41 and the second homology region comprises or consists of a nucleotide sequence that has at least 70% identity, at least 80% identity, at least 90% identity, at least 95% identity, at least 98% identity, or 100% identity to SEQ ID NO: 55; or
(xiii) the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the second homology region comprises or consists of a nucleotide sequence that has at least 70% identity, at least 80% identity, at least 90% identity, at least 95% identity, at least 98% identity, or 100% identity to SEQ ID NO: 57.

In some embodiments, the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the second homology region comprises or consists of a nucleotide sequence that has at least 70% identity, at least 80% identity, at least 90% identity, at least 95% identity, at least 98% identity, or 100% identity to SEQ ID NO: 57.

In some embodiments, the 3′ terminal sequence of the first homology region consists of a nucleotide sequence that has at least 70% identity, at least 80% identity, at least 90% identity, at least 95% identity, at least 98% identity, or 100% identity to any of SEQ ID NOs: 25-44 and/or the 5′ terminal sequence of the second homology region comprises or consists of a nucleotide sequence that has at least 70% identity, at least 80% identity, at least 90% identity, at least 95% identity, at least 98% identity, or 100% identity to any of SEQ ID NOs: 45-60.

Suitably, the 3′ terminal sequence of the first homology region comprises or consists of a nucleotide sequence that has at least 70% identity, at least 80% identity, at least 90% identity, at least 95% identity, at least 98% identity, or 100% identity to any of SEQ ID NOs: 44-60 and the 5′ terminal sequence of the second homology region comprises or consists of a nucleotide sequence that has at least 70% identity, at least 80% identity, at least 90% identity, at least 95% identity, at least 98% identity, or 100% identity to the corresponding nucleotide sequence Tables 3 or 4 (i.e. SEQ ID NOs: 45-60). For example, in some embodiments:
(i) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 25 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 45;
(ii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 26 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 46;
(iii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 27 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 47;
(iv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 28 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 48;
(v) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 49 or SEQ ID NO: 59;
(vi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 50 or SEQ ID NO: 60;
(vii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 31 or SEQ ID NO: 41 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 51;
(viii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 32 or SEQ ID NO: 42 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 52;
(ix) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 33 or SEQ ID NO: 42 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 53;
(x) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 34 or SEQ ID NO: 41 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 54;
(xi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 55;
(xii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 36 or SEQ ID NO: 42 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 56;
(xiii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 57; or
(xiv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 38 or SEQ ID NO: 44 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 58.

In some embodiments:
(v) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 49 or SEQ ID NO: 59; or
(vi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 50 or SEQ ID NO: 60.

In some embodiments:
(xi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 55; or
(xiii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 57.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 57.

Exemplary second homology regions for the exon 2 gene replacement strategies are shown below in Table 6.

In some embodiments, the second homology region comprises or consists of a nucleotide sequence that has at least 70% identity, at least 80% identity, at least 90% identity, at least 95% identity, at least 98% identity, or 100% identity to any of SEQ ID NOs: 61-68.

TABLE 6
Exemplary second homology regions for exon 2 gene replacement strategies

| Exon 2 region | Second homology region |
|---|---|
| chr11: 36574558-36574657 | Caaagcctttgctgacaaagaagaaggtggagatgtgaagtccgtgtgcatg |
| | accttgttcctgctggctctgagggcgaggaatgagcacaggcaagct (SEQ |
| | ID NO: 61) |
| chr11: 36574871-36574970 | cagccacctctgaagaatgtgtcttccagcactgatgttggcattattgatgggct |
| | gtctggactatcatcctctgtggatgattacccagtggacacca (SEQ ID |
| | NO: 62) |
| chr11: 36575184-36575283 | tcacaatcatgaaaattactattgcccacagctctcagaatgtgaaagtatttgaa |
| | gaagccaaacctaactctgaactgtgttgcaagccattgtgcct (SEQ ID |
| | NO: 63) |
| chr11: 36575497-36575596 | tctttgtgatgccacccgtctggaagcctctcaaaatcttgtcttccactctataacc |
| | agaagccatgctgagaacctggaacgttatgaggtctggcgt (SEQ ID |
| | NO: 64) |
| chr11: 36575811-36575910 | tggacaagcatctccggaagaagatgaacctcaaaccaatcatgaggatgaa |
| | tggcaactttgccaggaagctcatgaccaaagagactgtggatgcagt (SEQ |
| | ID NO: 65) |
| chr11: 36576124-36576223 | caaaaccctggcccatgttcctgaaattattgagagggatggctccattggggc |
| | atgggcaagtgagggaaatgagtctggtaacaaactgtttaggcgc (SEQ |
| | ID NO: 66) |
| chr11: 36576391-36576440 | aggcatagaggactctctggaaagccaagattcaatggaattttaagtag |
| | (SEQ ID NO: 67) |
| chr11: 36576437-36576536 | gtagggcaaccacttatgagttggtttttgcaattgagtttccctctgggttgcattg |
| | agggcttctcctagcaccctttactgctgtgtatggggcttc (SEQ ID NO: |
| | 68) |

In some embodiments:
(i) the first homology region comprises or consists of a nucleotide sequence that has at least 70% identity, at least 80% identity, at least 90% identity, at least 95% identity, at least 98% identity, or 100% identity to SEQ ID NO: 25 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(ii) the first homology region comprises or consists of a nucleotide sequence that has at least 70% identity, at least 80% identity, at least 90% identity, at least 95% identity, at least 98% identity, or 100% identity to SEQ ID NO: 26 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(iii) the first homology region comprises or consists of a nucleotide sequence that has at least 70% identity, at least 80% identity, at least 90% identity, at least 95% identity, at least 98% identity, or 100% identity to SEQ ID NO: 27 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(iv) the first homology region comprises or consists of a nucleotide sequence that has at least 70% identity, at least 80% identity, at least 90% identity, at least 95% identity, at least 98% identity, or 100% identity to SEQ ID NO: 28 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(v) the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(vi) the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(vii) the first homology region comprises or consists of a nucleotide sequence that has at least 70% identity, at least 80% identity, at least 90% identity, at least 95% identity, at least 98% identity, or 100% identity to SEQ ID NO: 31 or SEQ ID NO: 41 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(viii) the first homology region comprises or consists of a nucleotide sequence that has at least 70% identity, at least 80% identity, at least 90% identity, at least 95% identity, at least 98% identity, or 100% identity to SEQ ID NO: 32 or SEQ ID NO: 42 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(ix) the first homology region comprises or consists of a nucleotide sequence that has at least 70% identity, at least 80% identity, at least 90% identity, at least 95% identity, at least 98% identity, or 100% identity to SEQ ID NO: 33 or SEQ ID NO: 42 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(x) the first homology region comprises or consists of a nucleotide sequence that has at least 70% identity, at least 80% identity, at least 90% identity, at least 95% identity, at least 98% identity, or 100% identity to SEQ ID NO: 34 or SEQ ID NO: 41 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(xi) the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(xii) the first homology region comprises or consists of a nucleotide sequence that has at least 70% identity, at least 80% identity, at least 90% identity, at least 95% identity, at least 98% identity, or 100% identity to SEQ ID NO: 36 or SEQ ID NO: 42 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(xiii) the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the second homology region comprises or consists of a nucleotide sequence that has at least 70% identity, at least 80% identity, at least 90% identity, at least 95% identity, at least 98% identity, or 100% identity to any of SEQ ID NOs: 61-68; or
(xiv) the first homology region comprises or consists of a nucleotide sequence that has at least 70% identity, at least 80% identity, at least 90% identity, at least 95% identity, at least 98% identity, or 100% identity to SEQ ID NO: 38 or SEQ ID NO: 44 and the second homology region comprises or consists of a nucleotide sequence that has at least 70% identity, at least 80% identity, at least 90% identity, at least 95% identity, at least 98% identity, or 100% identity to any of SEQ ID NOs: 61-68.

In some embodiments:
(v) the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39 and the second homology region comprises or consists of a nucleotide sequence that has at least 70% identity, at least 80% identity, at least 90% identity, at least 95% identity, at least 98% identity, or 100% identity to any of SEQ ID NOs: 61-68; or
(vi) the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40 and the second homology region comprises or consists of a nucleotide sequence that has at least 70% identity, at least 80% identity, at least 90% identity, at least 95% identity, at least 98% identity, or 100% identity to any of SEQ ID NOs: 61-68.

In some embodiments:
(xi) the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41 and the second homology region comprises or consists of a nucleotide sequence that has at least 70% identity, at least 80% identity, at least 90% identity, at least 95% identity, at least 98% identity, or 100% identity to any of SEQ ID NOs: 61-68; or
(xiii) the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the second homology region comprises or consists of a nucleotide sequence that has at least 70% identity, at least 80% identity, at least 90% identity, at least 95% identity, at least 98% identity, or 100% identity to any of SEQ ID NOs: 61-68.

In some embodiments, the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the second homology region comprises or consists of a nucleotide sequence that has at least 70% identity, at least 80% identity, at least 90% identity, at least 95% identity, at least 98% identity, or 100% identity to any of SEQ ID NOs: 61-68.

In some embodiments:
(i) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 25 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(ii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 26 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(iii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 27 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(iv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 28 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(v) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(vi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(vii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 31 or SEQ ID NO: 41 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(viii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 32 or SEQ ID NO: 42 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(ix) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 33 or SEQ ID NO: 42 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(x) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 34 or SEQ ID NO: 41 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(xi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(xii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 36 or SEQ ID NO: 42 and the second homology region comprises or consists of a nucleotide sequence that has at least 70% identity, at least 80% identity, at least 90% identity, at least 95% identity, at least 98% identity, or 100% identity to any of SEQ ID NOs: 61-68;
(xiii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the second homology region comprises or consists of a nucleotide sequence that has at least 70% identity, at least 80% identity, at least 90% identity, at least 95% identity, at least 98% identity, or 100% identity to any of SEQ ID NOs: 61-68; or
(xiv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 38 or SEQ ID NO: 44 and the second homology region comprises or consists of a nucleotide sequence that has at least 70% identity, at least 80% identity, at least 90% identity, at least 95% identity, at least 98% identity, or 100% identity to any of SEQ ID NOs: 61-68.

In some embodiments:
(v) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39 and the second homology region comprises or consists of a nucleotide sequence that has at least 70% identity, at least 80% identity, at least 90% identity, at least 95% identity, at least 98% identity, or 100% identity to any of SEQ ID NOs: 61-68; or
(vi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40 and the second homology region comprises or consists of a nucleotide sequence that has at least 70% identity, at least 80% identity, at least 90% identity, at least 95% identity, at least 98% identity, or 100% identity to any of SEQ ID NOs: 61-68.

In some embodiments:
(xi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41 and the second homology region comprises or consists of a nucleotide sequence that has at least 70% identity, at least 80% identity, at least 90% identity, at least 95% identity, at least 98% identity, or 100% identity to any of SEQ ID NOs: 61-68; or
(xiii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the second homology region comprises or consists of a nucleotide sequence that has at least 70% identity, at least 80% identity, at least 90% identity, at least 95% identity, at least 98% identity, or 100% identity to any of SEQ ID NOs: 61-68.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the second homology region comprises or consists of a nucleotide sequence that has at least 70% identity, at least 80% identity, at least 90% identity, at least 95% identity, at least 98% identity, or 100% identity to any of SEQ ID NOs: 61-68.

In some embodiments:
(i) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 25 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(ii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 26 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(iii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 27 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(iv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 28 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(v) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(vi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(vii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 31 or SEQ ID NO: 41 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(viii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 32 or SEQ ID NO: 42 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(ix) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 33 or SEQ ID NO: 42 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(x) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 34 or SEQ ID NO: 41 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(xi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(xii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 36 or SEQ ID NO: 42 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67;
(xiii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67; or
(xiv) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 38 or SEQ ID NO: 44 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67.

In some embodiments:
(v) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 29 or SEQ ID NO: 39 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67; or
(vi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 30 or SEQ ID NO: 40 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67.

In some embodiments:
(xi) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 35 or SEQ ID NO: 41 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67; or
(xiii) the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67.

In some embodiments, the 3′ terminal sequence of the first homology region comprises or consists of a nucleotide sequence that has at least 70% identity, at least 80% identity, at least 90% identity, at least 95% identity, at least 98% identity, or 100% identity to SEQ ID NO: 37 or SEQ ID NO: 43 and the 5′ terminal sequence of the second homology region comprises or consists of a nucleotide sequence that has at least 70% identity, at least 80% identity, at least 90% identity, at least 95% identity, at least 98% identity, or 100% identity to SEQ ID NO: 67.

In some embodiments, the first homology region comprises or consists of a nucleotide sequence that has at least 70% identity, at least 80% identity, at least 90% identity, at least 95% identity, at least 98% identity, or 100% identity to any of SEQ ID NOs: 69-76 or 153-154, or a fragment thereof. Suitably, the fragments are at least 50 bp in length, for example 50-1000 bp or 100-500 bp in length.

In some embodiments, the second homology region comprises or consists of a nucleotide sequence that has at least 70% identity, at least 80% identity, at least 90% identity, at least 95% identity, at least 98% identity, or 100% identity to any of SEQ ID NOs: 77-78 or 155-156, or a fragment thereof. Suitably, the fragments are at least 50 bp in length, for example 50-1000 bp or 100-500 bp in length.

In some embodiments, the second homology region comprises or consists of a nucleotide sequence that has at least 70% identity, at least 80% identity, at least 90% identity, at least 95% identity, at least 98% identity, or 100% identity to any of SEQ ID NOs: 79-80 or 157, or a fragment thereof. Suitably, the fragments are at least 500 bp in length, for example 500-2000 bp or 900-1800 bp in length.

In some embodiments:
(1) the first homology region comprises or consists of a nucleotide sequence that has at least 70% identity, at least 80% identity, at least 90% identity, at least 95% identity, at least 98% identity, or 100% identity to SEQ ID NO: 69, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 77, or a fragment thereof;
(2) the first homology region comprises or consists of a nucleotide sequence that has at least 70% identity, at least 80% identity, at least 90% identity, at least 95% identity, at least 98% identity, or 100% identity to SEQ ID NO: 70, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof;
(3) the first homology region comprises or consists of a nucleotide sequence that has at least 70% identity, at least 80% identity, at least 90% identity, at least 95% identity, at least 98% identity, or 100% identity to SEQ ID NO: 70, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 80, or a fragment thereof;
(4) the first homology region comprises or consists of a nucleotide sequence that has at least 70% identity, at least 80% identity, at least 90% identity, at least 95% identity, at least 98% identity, or 100% identity to SEQ ID NO: 71, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 78, or a fragment thereof;
(5) the first homology region comprises or consists of a nucleotide sequence that has at least 70% identity, at least 80% identity, at least 90% identity, at least 95% identity, at least 98% identity, or 100% identity to SEQ ID NO: 72, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof;
(6) the first homology region comprises or consists of a nucleotide sequence that has at least 70% identity, at least 80% identity, at least 90% identity, at least 95% identity, at least 98% identity, or 100% identity to SEQ ID NO: 72, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 80, or a fragment thereof;
(7) the first homology region comprises or consists of a nucleotide sequence that has at least 70% identity, at least 80% identity, at least 90% identity, at least 95% identity, at least 98% identity, or 100% identity to SEQ ID NO: 73, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof;
(8) the first homology region comprises or consists of a nucleotide sequence that has at least 70% identity, at least 80% identity, at least 90% identity, at least 95% identity, at least 98% identity, or 100% identity to SEQ ID NO: 74, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof;
(9) the first homology region comprises or consists of a nucleotide sequence that has at least 70% identity, at least 80% identity, at least 90% identity, at least 95% identity, at least 98% identity, or 100% identity to SEQ ID NO: 75, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof; or
(10) the first homology region comprises or consists of a nucleotide sequence that has at least 70% identity, at least 80% identity, at least 90% identity, at least 95% identity, at least 98% identity, or 100% identity to SEQ ID NO: 76, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 79, or a fragment thereof.

In some embodiments:
(12) the first homology region comprises or consists of a nucleotide sequence that has at least 70% identity, at least 80% identity, at least 90% identity, at least 95% identity, at least 98% identity, or 100% identity to SEQ ID NO: 153, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 155, or a fragment thereof;
(13) the first homology region comprises or consists of a nucleotide sequence that has at least 70% identity, at least 80% identity, at least 90% identity, at least 95% identity, at least 98% identity, or 100% identity to SEQ ID NO: 153, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 157, or a fragment thereof;
(14) the first homology region comprises or consists of a nucleotide sequence that has at least 70% identity, at least 80% identity, at least 90% identity, at least 95% identity, at least 98% identity, or 100% identity to SEQ ID NO: 154, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 156, or a fragment thereof; or
(15) the first homology region comprises or consists of a nucleotide sequence that has at least 70% identity, at least 80% identity, at least 90% identity, at least 95% identity, at least 98% identity, or 100% identity to SEQ ID NO: 154, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 157, or a fragment thereof.

In some embodiments:
(14) the first homology region comprises or consists of a nucleotide sequence that has at least 70% identity, at least 80% identity, at least 90% identity, at least 95% identity, at least 98% identity, or 100% identity to SEQ ID NO: 154, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 156, or a fragment thereof; or
(15) the first homology region comprises or consists of a nucleotide sequence that has at least 70% identity, at least 80% identity, at least 90% identity, at least 95% identity, at least 98% identity, or 100% identity to SEQ ID NO: 154, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 157, or a fragment thereof.

In some embodiments, the first homology region comprises or consists of a nucleotide sequence that has at least 70% identity, at least 80% identity, at least 90% identity, at least 95% identity, at least 98% identity, or 100% identity to SEQ ID NO: 154, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 156, or a fragment thereof.

In some embodiments, the first homology region comprises or consists of a nucleotide sequence that has at least 70% identity, at least 80% identity, at least 90% identity, at least 95% identity, at least 98% identity, or 100% identity to SEQ ID NO: 154, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 157, or a fragment thereof.

In some embodiments:
(1) the first homology region comprises or consists of the nucleotide sequence of SEQ ID NO: 69, or a fragment thereof and the second homology region comprises or consists of the nucleotide sequence of SEQ ID NO: 77, or a fragment thereof;
(2) the first homology region comprises or consists of the nucleotide sequence of SEQ ID NO: 70, or a fragment thereof and the second homology region comprises or consists of the nucleotide sequence of SEQ ID NO: 79, or a fragment thereof;
(3) the first homology region comprises or consists of the nucleotide sequence of SEQ ID NO: 70, or a fragment thereof and the second homology region comprises or consists of the nucleotide sequence of SEQ ID NO: 80, or a fragment thereof;
(4) the first homology region comprises or consists of the nucleotide sequence of SEQ ID NO: 71, or a fragment thereof and the second homology region comprises or consists of the nucleotide sequence of SEQ ID NO: 78, or a fragment thereof;
(5) the first homology region comprises or consists of the nucleotide sequence of SEQ ID NO: 72, or a fragment thereof and the second homology region comprises or consists of the nucleotide sequence of SEQ ID NO: 79, or a fragment thereof;
(6) the first homology region comprises or consists of the nucleotide sequence of SEQ ID NO: 72, or a fragment thereof and the second homology region comprises or consists of the nucleotide sequence of SEQ ID NO: 80, or a fragment thereof;
(7) the first homology region comprises or consists of the nucleotide sequence of SEQ ID NO: 73, or a fragment thereof and the second homology region comprises or consists of the nucleotide sequence of SEQ ID NO: 79, or a fragment thereof;
(8) the first homology region comprises or consists of the nucleotide sequence of SEQ ID NO: 74, or a fragment thereof and the second homology region comprises or consists of the nucleotide sequence of SEQ ID NO: 79, or a fragment thereof;
(9) the first homology region comprises or consists of the nucleotide sequence of SEQ ID NO: 75, or a fragment thereof and the second homology region comprises or consists of the nucleotide sequence of SEQ ID NO: 79, or a fragment thereof; or
(10) the first homology region comprises or consists of the nucleotide sequence of SEQ ID NO: 76, or a fragment thereof and the second homology region comprises or consists of the nucleotide sequence of SEQ ID NO: 79, or a fragment thereof.

In some embodiments:
(12) the first homology region comprises or consists of the nucleotide sequence of SEQ ID NO: 153, or a fragment thereof and the second homology region comprises or consists of the nucleotide sequence of SEQ ID NO: 155, or a fragment thereof;
(13) the first homology region comprises or consists of the nucleotide sequence of SEQ ID NO: 153, or a fragment thereof and the second homology region comprises or consists of the nucleotide sequence of SEQ ID NO: 157, or a fragment thereof;
(14) the first homology region comprises or consists of the nucleotide sequence of SEQ ID NO: 154, or a fragment thereof and the second homology region comprises or consists of the nucleotide sequence of SEQ ID NO: 156, or a fragment thereof; or
(15) the first homology region comprises or consists of the nucleotide sequence of SEQ ID NO: 154, or a fragment thereof and the second homology region comprises or consists of the nucleotide sequence of SEQ ID NO: 157, or a fragment thereof.

In some embodiments:
(14) the first homology region comprises or consists of the nucleotide sequence of SEQ ID NO: 154, or a fragment thereof and the second homology region comprises or consists of the nucleotide sequence of SEQ ID NO: 156, or a fragment thereof; or
(15) the first homology region comprises or consists of the nucleotide sequence of SEQ ID NO: 154, or a fragment thereof and the second homology region comprises or consists of the nucleotide sequence of SEQ ID NO: 157, or a fragment thereof.

In some embodiments, the first homology region comprises or consists of the nucleotide sequence of SEQ ID NO: 154, or a fragment thereof and the second homology region comprises or consists of the nucleotide sequence of SEQ ID NO: 156, or a fragment thereof.

In some embodiments, the first homology region comprises or consists of the nucleotide sequence of SEQ ID NO: 154, or a fragment thereof and the second homology region comprises or consists of the nucleotide sequence of SEQ ID NO: 157, or a fragment thereof.

Illustrative first homology region for g5 exon 2
(SEQ ID NO: 69)
gatccatcaagccaaccttcgacatctctgccgcatctgtgggaattcttttagagctgatgagcacaacaggagatatc
cagtccatggtcctgtggatggtaaaaccctaggccttttacgaaagaaggaaaagagagctacttcctggccggac
ctcattgccaaggttttccggatcgatgtgaaggcagatgttgactcgatccaccccactgagttctgccataactgctgg
agcatcatgcacaggaagtttagcagtgccccatgtgaggtttacttcccgaggaacgtgaccatggagtggcacccc
cacacaccatcctgtgacatctgcaacactgcccgtcggggactcaagaggaagagtcttcagccaaacttgcagct
cagcaaaaaactcaaaactgtgcttgaccaagcaagacaagcccgtcagcgcaagagaagagctcaggcaagg
atcagcagcaaggatgtcatgaagaagatcgcaaact
Illustrative first homology region for g5 exon 2
(SEQ ID NO: 70)
aaaaccctaggccttttacgaaagaaggaaaagagagctacttcctggccggacctcattgccaaggttttccggatc
gatgtgaaggcagatgttgactcgatccaccccactgagttctgccataactgctggagcatcatgcacaggaagttta
gcagtgccccatgtgaggtttacttcccgaggaacgtgaccatggagtggcacccccacacaccatcctgtgacatct
gcaacactgcccgtcggggactcaagaggaagagtcttcagccaaacttgcagctcagcaaaaaactcaaaactgt
gcttgaccaagcaagacaagcccgtcagcgcaagagaagagctcaggcaaggatcagcagcaaggatgtcatg
aagaagatcgcaaact
Illustrative first homology region for g6 exon 2
(SEQ ID NO: 71)
tgagatcctttgaaaagacacctgaagaagctcaaaaggaaaagaaggattcctttgaggggaaaccctctctgga
gcaatctccagcagtcctggacaaggctgatggtcagaagccagtcccaactcagccattgttaaaagcccacccta
agttttcaaagaaatttcacgacaacgagaaagcaagaggcaaagcgatccatcaagccaaccttcgacatctctg
ccgcatctgtgggaattcttttagagctgatgagcacaacaggagatatccagtccatggtcctgtggatggtaaaacc
ctaggccttttacgaaagaaggaaaagagagctacttcctggccggacctcattgccaaggttttccggatcgatgtga
aggcagatgttgactcgatccaccccactgagttctgccataactgctggagcatcatgcacaggaagtttagcagtgc
cccatgtgaggtttacttcccgaggaatgtcactatg
Illustrative first homology region for g6 exon 2
(SEQ ID NO: 72)
gagcacaacaggagatatccagtccatggtcctgtggatggtaaaaccctaggccttttacgaaagaaggaaaaga
gagctacttcctggccggacctcattgccaaggttttccggatcgatgtgaaggcagatgttgactcgatccaccccact
gagttctgccataactgctggagcatcatgcacaggaagtttagcagtgccccatgtgaggtttacttcccgaggaatgt
cactatg
Illustrative first homology region for g7, g10, g13 exon 2
(SEQ ID NO: 73)
gaattcttttagagctgatgagcacaacaggagatatccagtccatggtcctgtggatggtaaaaccctaggccttttac
gaaagaaggaaaagagagctacttcctggccggacctcattgccaaggttttccggatcgatgtgaaggcagatgtt
gactcgatccaccccactgagttctgccataactgctggagcatcatgcacaggaagtttagcagtgcaccat
Illustrative first homology region for g8, g9, g12 exon 2
(SEQ ID NO: 74)
ccagtccatggtcctgtggatggtaaaaccctaggccttttacgaaagaaggaaaagagagctacttcctggccgga
cctcattgccaaggttttccggatcgatgtgaaggcagatgttgactcgatccaccccactgagttctgccataactgctg
gagcatcatgcacaggaagtttagcagtgccccatgtgaggtttacttcccgaggaacgtgaccatggagtggcacc
cccacacaccatcctgtgacatctgcaacactgc
Illustrative first homology region for g11 exon 2
(SEQ ID NO: 75)
ctgatgagcacaacaggagatatccagtccatggtcctgtggatggtaaaaccctaggccttttacgaaagaaggaa
aagagagctacttcctggccggacctcattgccaaggttttccggatcgatgtgaaggcagatgttgactcgatccacc
ccactgagttctgccataactgctggagcatcatgcacaggaagtttagcagtgccccatgtgaggtttacttc
Illustrative first homology region for g14 exon 2
(SEQ ID NO: 76)
catggagtggcacccccacacaccatcctgtgacatctgcaacactgcccgtcggggactcaagaggaagagtcttc
agccaaacttgcagctcagcaaaaaactcaaaactgtgcttgaccaagcaagacaagcccgtcagcgcaagaga
agagctcaggcaaggatcagcagcaaggatgtcatgaagaagatcgccaactgcagtaagatacatcttagtacca
agctccttgcagtggacttcccagagcactttgtgaaatccatctcctgccagatctgtgaacacattctggctgaccctg
tggagaccaactgtaagcatgtcttttgccgggtctgcattctcagatgcctcaaagtcatgggcagctattgtccctcttg
ccgatatccatgcttccctactgacctggagagtccagtgaagtcctttctgagcgtcttgaattccctgatggtgaaatgt
ccagcaaaagagtgcaatgaggaggtca
Illustrative first homology region for g11 exon 2
(SEQ ID NO: 153)
ttcagcacccacatattaaattttcagaatggaaatttaagctgttccgggtgagatcctttgaaaagacacctgaagaa
gctcaaaaggaaaagaaggattcctttgaggggaaaccctctctggagcaatctccagcagtcctggacaaggctg
atggtcagaagccagtcccaactcagccattgttaaaagcccaccctaagttttcaaagaaatttcacgacaacgaga
aagcaagaggcaaagcgatccatcaagccaaccttcgacatctctgccgcatctgtgggaattcttttagagctgatg
agcacaacaggagatatccagtccatggtcctgtggatggtaaaaccctaggccttttacgaaagaaggaaaagag
agctacttcctggccggacctcattgccaaggttttccggatcgatgtgaaggcagatgttgactcgatccaccccactg
agttctgccataactgctggagcatcatgcacaggaagtttagcagtgccccatgtgaggtttacttc
Illustrative first homology region for g7, g10, g13 exon 2
(SEQ ID NO: 154)
ttcagcacccacatattaaattttcagaatggaaatttaagctgttccgggtgagatcctttgaaaagacacctgaagaa
gctcaaaaggaaaagaaggattcctttgaggggaaaccctctctggagcaatctccagcagtcctggacaaggctg
atggtcagaagccagtcccaactcagccattgttaaaagcccaccctaagttttcaaagaaatttcacgacaacgaga
aagcaagaggcaaagcgatccatcaagccaaccttcgacatctctgccgcatctgtgggaattcttttagagctgatg
agcacaacaggagatatccagtccatggtcctgtggatggtaaaaccctaggccttttacgaaagaaggaaaagag
agctacttcctggccggacctcattgccaaggttttccggatcgatgtgaaggcagatgttgactcgatccaccccactg
agttctgccataactgctggagcatcatgcacaggaagtttagcagtgcaccat
Illustrative second homology region for g5 exon 2-targeting strategy
(SEQ ID NO: 77)
gcagtaagatacatcttagtaccaagctccttgcagtggacttcccagagcactttgtgaaatccatctcctgccagatct
gtgaacacattctggctgaccctgtggagaccaactgtaagcatgtcttttgccgggtctgcattctcagatgcctcaaag
tcatgggcagctattgtccctcttgccgatatccatgcttccctactgacctggagagtccagtgaagtcctttctgagcgt
cttgaa
Illustrative second homology region for g6 exon 2-targeting strategy
(SEQ ID NO: 78)
gagtggcacccccacacaccatcctgtgacatctgcaacactgcccgtcggggactcaagaggaagagtcttcagc
caaacttgcagctcagcaaaaaactcaaaactgtgcttgaccaagcaagacaagcccgtcagcgcaagagaaga
gctcaggcaaggatcagcagcaaggatgtcatgaagaagatcgccaactgcagtaagatacatcttagtaccaagc
tccttgcagtggacttcccagagc
Illustrative second homology region for exon 2-replacement strategy
(SEQ ID NO: 79)
aggcatagaggactctctggaaagccaagattcaatggaattttaagtagggcaaccacttatgagttggtttttgcaatt
gagtttccctctgggttgcattgagggcttctcctagcaccctttactgctgtgtatggggcttcaccatccaagaggtggta
ggttggagtaagatgctacagatgctctcaagtcaggaatagaaactgatgagctgattgcttgaggcttttagtgagttc
cgaaaagcaacaggaaaaatcagttatctgaaagctcagtaactcagaacaggagtaactgcaggggaccagag
atgagcaaagatctgtgtgtgttggggagctgtcatgtaaatcaaagccaaggttgtcaaagaacagccagtgaggc
caggaaagaaattggtcttgtggttttcatttttttcccccttgattgattatattttgtattgagatatgataagtgccttctatttc
atttttgaataattcttcatttttataattttacatatcttggcttgctatataagattcaaaagagctttttaaatttttctaataatat
cttacatttgtacagcatgatgacctttacaaagtgctctcaatgcatttacccattcgttatataaatatgttacatcaggac
aactttgagaaaatcagtccttttttatgtttaaattatgtatctattgtaaccttcagagtttaggaggtcatctgctgtcatgg
atttttcaataatgaatttagaatacacctgttagctacagttagttattaaatcttctgataatatatgtttacttagctatcaga
agccaagtatgattctttatttttactttttcatttcaagaaatttagagtttccaaatttagagcttctgcatacagtcttaaagc
cacagaggcttgtaaaaatataggttagcttgatgtctaaaaatatatttcatgtcttactgaaacattttgccagactttctc
caaatgaaacctgaatcaatttttctaaatctaggtttcatagagtcctctcctctgcaatgtgttattctttctataatgatcag
tttactttcagtggattcagaattgtgtagcaggataaccttgtatttttccatccgctaagtttagatggagtccaaacgca
gtacagcagaagagttaacatttacacagtgctttttaccactgtggaatgttttcacactcatttttccttacaacaattctg
aggagtaggtgttgttattatctccatttgatgggggtttaaatgatttgctcaaagtcatttaggggtaataaatacttggctt
ggaaatttaacacagtccttttgtctccaaagcccttcttctttccaccacaaattaatcactatgtttataaggtagtatcag
aatttttttaggattcacaactaatcactatagcacatgaccttgggattacatttttatggggcaggggtaagcaagttttta
aatcatttgtgtgctctggctcttttgatagaagaaagcaacacaaaagctccaaagggccccctaaccctcttgtggct
ccagttatttggaaactatgatctgcatccttaggaatctgggatttgccagttgctggcaatgtagagcaggcatggaat
tttatatgctagtgagtcataatgatatgttagtgttaattagttttttcttcctttgattttattggccataattgctactcttcataca
cagtatatcaaagagcttgataatttagttgtcaaaag
Illustrative second homology region for exon 2-replacement strategy
(SEQ ID NO: 80)
aggcatagaggactctctggaaagccaagattcaatggaattttaagtagggcaaccacttatgagttggtttttgcaatt
gagtttccctctgggttgcattgagggcttctcctagcaccctttactgctgtgtatggggcttcaccatccaagaggtggta
ggttggagtaagatgctacagatgctctcaagtcaggaatagaaactgatgagctgattgcttgaggcttttagtgagttc
cgaaaagcaacaggaaaaatcagttatctgaaagctcagtaactcagaacaggagtaactgcaggggaccagag
atgagcaaagatctgtgtgtgttggggagctgtcatgtaaatcaaagccaaggttgtcaaagaacagccagtgaggc
caggaaagaaattggtcttgtggttttcatttttttcccccttgattgattatattttgtattgagatatgataagtgccttctatttc
atttttgaataattcttcatttttataattttacatatcttggcttgctatataagattcaaaagagctttttaaatttttctaataatat
cttacatttgtacagcatgatgacctttacaaagtgctctcaatgcatttacccattcgttatataaatatgttacatcaggac
aactttgagaaaatcagtccttttttatgtttaaattatgtatctattgtaaccttcagagtttaggaggtcatctgctgtcatgg
atttttcaataatgaatttagaatacacctgttagctacagttagttattaaatcttctgataatatatgtttacttagctatcaga
agccaagtatgattctttatttttactttttcatttcaagaaatttagagtttccaaatttagagct
Illustrative second homology region for g11 exon 2-targeting strategy
(SEQ ID NO: 155)
ccgaggaacgtgaccatggagtggcacccccacacaccatcctgtgacatctgcaacactgcccgtcggggactca
agaggaagagtcttcagccaaacttgcagctcagcaaaaaactcaaaactgtgcttgaccaagcaagacaagccc
gtcagcgcaagagaagagctcaggcaaggatcagcagcaaggatgtcatgaagaagatcgccaactgcagtaa
gatacatcttagtaccaagctccttgcagtggacttcccagagcactttgtgaaatccatctcctgccagatctgtgaaca
cattctggctgaccctgtggagaccaactgtaagcatgtcttttgccgggtctgcattctcagatgcctcaaagtcatggg
cagctattgtccctcttgccgatatccatgcttccctactgacctggagagtccagtgaagtcctttctgagcgtcttgaatt
ccctgatggtgaaatgtccagcaaaagagtg
Illustrative second homology region for g13 exon 2-targeting strategy
(SEQ ID NO: 156)
gtgaggtttacttcccgaggaacgtgaccatggagtggcacccccacacaccatcctgtgacatctgcaacactgccc
gtcggggactcaagaggaagagtcttcagccaaacttgcagctcagcaaaaaactcaaaactgtgcttgaccaagc
aagacaagcccgtcagcgcaagagaagagctcaggcaaggatcagcagcaaggatgtcatgaagaagatcgcc
aactgcagtaagatacatcttagtaccaagctccttgcagtggacttcccagagcactttgtgaaatccatctcctgcca
gatctgtgaacacattctggctgaccctgtggagaccaactgtaagcatgtcttttgccgggtctgcattctcagatgcctc
aaagtcatgggcagctattgtccctcttgccgatatccatgcttccctactgacctggagagtccagtgaagtcctttctga
gcgtcttgaattccctgatggtgaaatgt
Illustrative second homology region for exon 2-replacement strategy
(SEQ ID NO: 157)
aggcatagaggactctctggaaagccaagattcaatggaattttaagtagggcaaccacttatgagttggtttttgcaatt
gagtttccctctgggttgcattgagggcttctcctagcaccctttactgctgtgtatggggcttcaccatccaagaggtggta
ggttggagtaagatgctacagatgctctcaagtcaggaatagaaactgatgagctgattgcttgaggcttttagtgagttc
cgaaaagcaacaggaaaaatcagttatctgaaagctcagtaactcagaacaggagtaactgcaggggaccagag
atgagcaaagatctgtgtgtgttggggagctgtcatgtaaatcaaagccaaggttgtcaaagaacagccagtgaggc
caggaaagaaattggtcttgtggttttcatttttttcccccttgattgattatattttgtattgagatatgataagtgccttctatttc
atttttgaataattcttcatttttataattttacatatcttggcttgctatataagattcaaaagagctttttaaatttttctaataatat
cttacatttgtacagcatgatgacctttacaaagtgctctcaatgcatttacccattcgttatataaatatgttacatcaggac
aactttgagaaaatcagtccttttttatgtttaaattatgtatctattgtaaccttcagagtttaggaggtcatctgctgtcatgg
atttttcaataatgaatttagaatacacctgttagctacagttagttattaaatcttctgataatatatgtttacttagctatcaga
agccaagtatgattctttatttttactttttcatttcaagaaatttagagtttccaaatttagagcttctgcatacagtcttaaagc
cacagaggcttgtaaaaatataggttagcttgatgtctaaaaatatatttcatgtcttactgaaacattttgccagactttctc
caaatgaaacctgaatcaatttttctaaatctaggtttcatagagtcctctcctctgcaatgtgttattctttctataatgatcag
tttactttcagtggattcagaattgtgtagcaggataaccttgtatttttccatccgctaagtttagatggagtccaaacgca
gtacagcagaagagtt

### Intron 1 Strategies

In some embodiments, the first homology region is homologous to a first region of the RAG1 intron 1 or the start of the RAG1 exon 2 (e.g. the first 200 bp of the RAG1 exon 2) and the second homology region is homologous to a second region of the RAG1 exon 2.

In some embodiments, the first homology region is homologous to a region of the RAG1 intron 1 and the second homology region is homologous to a region of the RAG1 exon 2.

In some embodiments, the first homology region is homologous to a region upstream of: (i) chr 11:36569295; (ii) chr 11:36573790; (iii) chr 11:36573641; (iv) chr 11:36573351; (v) chr 11:36569080; (vi) chr 11:36572472; (vii) chr 11:36571458; (viii) chr 11:36571366; (ix) chr 11:36572859 (x) chr 11:36571457; (xi) chr 11:36569351; or (xii) chr 11:36572375.

In some embodiments, the first homology region is homologous to a region upstream of: (i) chr 11:36569295; (ii) chr 11:36573351; (iii) chr 11:36571366 In some embodiments, the first homology region is homologous to a region upstream of chr 11:36569295.

In some embodiments: (i) the first homology region is homologous to a region comprising chr 11:36569245-36569294; (ii) the first homology region is homologous to a region comprising chr 11:36573740-36573789; (iii) the first homology region is homologous to a region comprising chr 11:36573591-36573640; (iv) the first homology region is homologous to a region comprising chr 11:36573301-36573350; (v) the first homology region is homologous to a region comprising chr 11:36569030-36569079; (vi) the first homology region is homologous to a region comprising chr 11:36572422-36572471; (vii) the first homology region is homologous to a region comprising chr 11:36571408-36571457; (viii) the first homology region is homologous to a region comprising chr 11:36571316-36571365; (ix) the first homology region is homologous to a region comprising chr 11:36572809-36572858; (x) the first homology region is homologous to a region comprising chr 11:36571407-36571456; (xi) the first homology region is homologous to a region comprising chr 11:36569301-36569350; or (xii) the first homology region is homologous to a region comprising chr 11:36572325-36572374.

In some embodiments: (i) the first homology region is homologous to a region comprising chr 11:36569245-36569294; (ii) the first homology region is homologous to a region comprising chr 11:36573301-36573350; or (iii) the first homology region is homologous to a region comprising chr 11:36571316-36571365.

In some embodiments, the first homology region is homologous to a region comprising chr 11:36569245-36569294.

Exemplary first homology regions for intron 1 strategies are shown below in Table 7.

In some embodiments, the first homology region comprises or consists of a nucleotide sequence that has at least 70% identity, at least 80% identity, at least 90% identity, at least 95% identity, at least 98% identity, or 100% identity to any of SEQ ID NOs: 81-92.

TABLE 7
Exemplary first homology regions for intron 1
strategies

| Guide RNA | First homology region |
|---|---|
| 9 | TGCTGTGTGGAGGGAGGCACGCCTGTAGCTCT |
| | GATGTCAGATGGCAATGT (SEQ ID NO: 81) |
| 1 | AAGAGAGCTACTTCCTGGCCGGACCTCATTGC |
| | CAAGGTTTTCCGGATCGA (SEQ ID NO: 82) |
| 2 | AAGCAAGAGGCAAAGCGATCCATCAAGCCAAC |
| | CTTCGACATCTCTGCCGC (SEQ ID NO: 83) |
| 3 | CAGCATGGCAGCCTCTTTCCCACCCACCTTGG |
| | GACTCAGTTCTGCCCCAG (SEQ ID NO: 84) |
| 4 | TTGTGTACAGACTAAGTTGAAGATGTTAGGAGG |
| | GAAGATTGTGGGCCAAG (SEQ ID NO: 85) |
| 5 | TTACTCCCACCTCTTCTTATTATGTTACAAACTA |
| | TAGTGCTAATGACCAT (SEQ ID NO: 86) |
| 6 | ACAGAAGAGAATTAGGAAGCAGAATTGAACTAT |
| | AAGCAATTTTGAGGTGT (SEQ ID NO: 87) |
| 7 | GGGAAGTAAAATGCTAAAGGAATGAGAAGGCA |
| | TTTGGGGTTGAGTTCAAC (SEQ ID NO: 88) |
| 8 | AACCAACCCCCTGGAAGACTGCTTTAAAAAGCT |
| | GGAAATACATTGTCCAG (SEQ ID NO: 89) |
| 10 | CACAGAAGAGAATTAGGAAGCAGAATTGAACTA |
| | TAAGCAATTTTGAGGTG (SEQ ID NO: 90) |
| 11 | GGCAGTGGCCGGTGGGGACAGGGCTGAGCCA |
| | GCACCAACCACTCAGCCTT (SEQ ID NO: 91) |
| 12 | TTTTTTCTGCATCGCTAGCGATCTGTGCATTAC |
| | AACTCAAATCAGTCGGG (SEQ ID NO: 92) |

In some embodiments:
(i) the first homology region comprises or consists of a nucleotide sequence that has at least 70% identity, at least 80% identity, at least 90% identity, at least 95% identity, at least 98% identity, or 100% identity to SEQ ID NO: 81;
(ii) the first homology region comprises or consists of a nucleotide sequence that has at least 70% identity, at least 80% identity, at least 90% identity, at least 95% identity, at least 98% identity, or 100% identity to SEQ ID NO: 84; or
(iii) the first homology region comprises or consists of a nucleotide sequence that has at least 70% identity, at least 80% identity, at least 90% identity, at least 95% identity, at least 98% identity, or 100% identity to SEQ ID NO: 88.

In some embodiments, the first homology region comprises or consists of a nucleotide sequence that has at least 70% identity, at least 80% identity, at least 90% identity, at least 95% identity, at least 98% identity, or 100% identity to SEQ ID NO: 81.

In some embodiments, the first homology region comprises or consists of a nucleotide sequence that has at least 98% identity to SEQ ID NO: 81.

In some embodiments, the first homology region comprises or consists of the nucleotide sequence of SEQ ID NO: 81.

In some embodiments, the 3′ terminal sequence of the first homology region consists of a nucleotide sequence that has at least 70% identity, at least 80% identity, at least 90% identity, at least 95% identity, at least 98% identity, or 100% identity to any of SEQ ID NOs: 81-92.

In some embodiments:
(i) the 3′ terminal sequence of the first homology region consists of a nucleotide sequence that has at least 70% identity, at least 80% identity, at least 90% identity, at least 95% identity, at least 98% identity, or 100% identity to SEQ ID NO: 81;
(ii) the 3′ terminal sequence of the first homology region consists of a nucleotide sequence that has at least 70% identity, at least 80% identity, at least 90% identity, at least 95% identity, at least 98% identity, or 100% identity to SEQ ID NO: 84; or
(iii) the 3′ terminal sequence of the first homology region consists of a nucleotide sequence that has at least 70% identity, at least 80% identity, at least 90% identity, at least 95% identity, at least 98% identity, or 100% identity to SEQ ID NO: 88.

In some embodiments, the 3′ terminal sequence of the first homology region consists of a nucleotide sequence that has at least 70% identity, at least 80% identity, at least 90% identity, at least 95% identity, at least 98% identity, or 100% identity to SEQ ID NO: 81.

In some embodiments, the 3′ terminal sequence of the first homology region consists of a nucleotide sequence that has at least 98% identity to SEQ ID NO: 81.

In some embodiments, the 3′ terminal sequence of the first homology region consists of the nucleotide sequence of SEQ ID NO: 81.

In some embodiments, the first homology region comprises or consists of a nucleotide sequence that has at least 70% identity, at least 80% identity, at least 90% identity, at least 95% identity, at least 98% identity, or 100% identity to SEQ ID NO: 93, or a fragment thereof. Suitably, the fragment is at least 50 bp in length, for example 50-250 bp or 100-200 bp in length.

In some embodiments, the first homology region comprises or consists of a nucleotide sequence that has at least 98% identity to SEQ ID NO: 93, or a fragment thereof.

In some embodiments, the first homology region comprises or consists of the nucleotide sequence of SEQ ID NO: 93.

Illustrative first homology region for guide RNA 9
(SEQ ID NO: 93)
tgagcacacagttattacttggaaattgtgtacagactaagttgaagatgttaggagggaagattgtgggccaagtaac
ggggtgtatgtgtgtgggtatagggtgggcagctgggatggaaatggggggctgctgctgctgctgcaccctggcctc
ctgaactaatgatatcactcaccagaaactactgttcctgcactgtccaagccaccccaaactagtttgtcaaaatgaat
ctgtgctgtgtggagggaggcacgcctgtagctctgatgtcagatggcaatgt

The second homology region may be homologous to a region distantly downstream of the DSB.

Suitable second homology regions which are homologous to a region distantly downstream of the DSB are described above for the “exon 2 RAG1 gene replacement strategy” (see e.g. Table 6). Any suitable second homology region described above may be used in the “exon 2 RAG1 gene replacement strategy” may also be used in the “intron 1 RAG1 gene replacement strategy” and vice versa

In some embodiments, the second homology region comprises or consists of a nucleotide sequence that has at least 70% identity, at least 80% identity, at least 90% identity, at least 95% identity, at least 98% identity, or 100% identity to SEQ ID NO: 94, or a fragment thereof. Suitably, the fragment is at least 500 bp in length, for example 500-2000 bp or 900-1800 bp in length.

In some embodiments, the second homology region comprises or consists of a nucleotide sequence that has at least 98% identity to SEQ ID NO: 94, or a fragment thereof.

In some embodiments, the second homology region comprises or consists of the nucleotide sequence of SEQ ID NO: 94, or a fragment thereof.

Illustrative second homology region for intron 1-replacement strategy
(SEQ ID NO: 94)
aggcatagaggactctctggaaagccaagattcaatggaattttaagtagggcaaccacttatgagttggtttttgcaatt
gagtttccctctgggttgcattgagggcttctcctagcaccctttactgctgtgtatggggcttcaccatccaagaggtggta
ggttggagtaagatgctacagatgctctcaagtcaggaatagaaactgatgagctgattgcttgaggcttttagtgagttc
cgaaaagcaacaggaaaaatcagttatctgaaagctcagtaactcagaacaggagtaactgcaggggaccagag
atgagcaaagatctgtgtgtgttggggagctgtcatgtaaatcaaagccaaggttgtcaaagaacagccagtgaggc
caggaaagaaattggtcttgtggttttcatttttttcccccttgattgattatattttgtattgagatatgataagtgccttctatttc
atttttgaataattcttcatttttataattttacatatcttggcttgctatataagattcaaaagagctttttaaatttttctaataatat
cttacatttgtacagcatgatgacctttacaaagtgctctcaatgcatttacccattcgttatataaatatgttacatcaggac
aactttgagaaaatcagtccttttttatgtttaaattatgtatctattgtaaccttcagagtttaggaggtcatctgctgtcatgg
atttttcaataatgaatttagaatacacctgttagctacagttagttattaaatcttctgataatatatgtttacttagctatcaga
agccaagtatgattctttatttttactttttcatttcaagaaatttagagtttccaaatttagagcttctgcatacagtcttaaagc
cacagaggcttgtaaaaatataggttagcttgatgtctaaaaatatatttcatgtcttactgaaacattttgccagactttctc
caaatgaaacctgaatcaatttttctaaatctaggtttcatagagtcctctcctctgcaatgtgttattctttctataatgatcag
tttactttcagtggattcagaattgtgtagcaggataaccttgtatttttccatccgctaagtttagatggagtccaaacgca
gtacagcagaagagttaacatttacacagtgctttttaccactgtggaatgttttcacactcatttttccttacaacaattctg
aggagtaggtgttgttattatctccatttgatgggggtttaaatgatttgctcaaagtcatttaggggtaataaatacttggctt
ggaaatttaacacagtccttttgtctccaaagcccttcttctttccaccacaaattaatcactatgtttataaggtagtatcag
aatttttttaggattcacaactaatcactatagcacatgaccttgggattacatttttatggggcaggggtaagcaagttttta
aatcatttgtgtgctctggctcttttgatagaagaaagcaacacaaaagctccaaagggccccctaaccctcttgtggct
ccagttatttggaaactatgatctgcatccttaggaatctgggatttgccagttgctggcaatgtagagcaggcatggaat
tttatatgctagtgagtcataatgatatgttagtgttaattagttttttcttcctttgattttattggccataattgctactcttcataca
cagtatatcaaagagcttgataatttagtt

In some embodiments, the first homology region comprises or consists of a nucleotide sequence that has at least 70% identity, at least 80% identity, at least 90% identity, at least 95% identity, at least 98% identity, or 100% identity to SEQ ID NO: 93, or a fragment thereof and the second homology region comprises or consists of a nucleotide sequence that has at least 70% identity, at least 80% identity, at least 90% identity, at least 95% identity, at least 98% identity, or 100% identity to SEQ ID NO: 94, or a fragment thereof.

In some embodiments, the first homology region comprises or consists of a nucleotide sequence that has at least 98% identity to SEQ ID NO: 93, or a fragment thereof and the second homology region comprises or consists of a nucleotide sequence that has at least 98% identity to SEQ ID NO: 94, or a fragment thereof.

In some embodiments, the first homology region comprises or consists of the nucleotide sequence of SEQ ID NO: 93, or a fragment thereof and the second homology region comprises or consists of the nucleotide sequence of SEQ ID NO: 94, or a fragment thereof.

### Genome Insertion Sites

The site of the double-strand break (DSB) can be introduced specifically by any suitable technique, for example using a CRISPR/Cas9 system and the guide RNAs disclosed herein. In the present invention, the DSB is introduced into the RAG1 intron 1 or RAG1 exon 2. For example, a DSB may be introduced at any of the sites recited in Tables 8 or 11 below.

Suitably, each homology region is homologous to a fragment of the RAG1 gene either side of the DSB. For example, the first homology region may be homologous to a region upstream of the DSB and the second homology region may be homologous to a region downstream of the DSB. The first homology region may be homologous to a region immediately upstream of the DSB and the second homology region may be homologous to either (a) a region immediately downstream of the DSB; or (b) a region distantly downstream of the DSB.

In the present invention, the nucleotide sequence insert (e.g. a nucleotide sequence encoding a RAG1 polypeptide or a RAG1 polypeptide fragment) may be introduced at the DSB site by homology-directed repair (HDR). Thus, the nucleotide insert (e.g. a nucleotide sequence encoding a RAG1 polypeptide or a RAG1 polypeptide fragment) may replace the region of the genome flanked by the homology regions and comprising the DSB.

As used herein, the “nucleotide sequence insert” may consist of the region of the polynucleotide flanked by the first homology region and the second homology region. For example, the nucleotide sequence insert may comprise a nucleotide sequence encoding a RAG1 polypeptide fragment. In some embodiments, the nucleotide sequence insert may comprise a splice acceptor sequence and a nucleotide sequence encoding a RAG1 polypeptide or a RAG1 polypeptide fragment.

### Exon 2 Strategies

In some embodiments, a DSB is introduced into the RAG1 exon 2 (e.g. in the exon 2 strategies discussed above). For example, a DSB may be introduced at any of the sites recited in Table 8 below.

TABLE 8
Exemplary DSB sites in RAG1 exon 2 (for exon 2 strategies)

| Guide | Exemplary DSB site |
|---|---|
| g1 M5 ex2 | between chr 11: 36574368 and 36574369 |
| g2 M5 ex2 | between chr 11: 36574367 and 36574368 |
| g3 M5 ex2 | between chr 11: 36574394 and 36574395 |
| g4 M4 ex2 | between chr 11: 36574294 and 36574295 |
| g5 M3 ex2 | between chr 11: 36574109 and 36574110 |
| g6 M2 ex2 | between chr 11: 36573910 and 36573911 |
| g7 exon2 M2/3 | between chr 11: 36573878 and 36573879 |
| g8 exon2 M2/3 | between chr 11: 36573959 and 36573960 |
| g9 exon2 M2/3 | between chr 11: 36573957 and 36573958 |
| g10 exon2 M2/3 | between chr 11: 36573879 and 36573880 |
| g11 exon2 M2/3 | between chr 11: 36573892 and 36573893 |
| g12 exon2 M2/3 | between chr 11: 36573955 and 36573956 |
| g13 exon2 M2/3 | between chr 11: 36573878 and 36573879 |
| g14 exon2 M5 | between chr 11: 36574406 and 36574407 |

The nucleotide sequence insert may be introduced into a genome at any of the sites recited in Table 8 above. In other words, the genome of the present invention may comprise the nucleotide sequence insert at any of the sites recited in Table 8 above.

In preferred embodiments, a nucleotide sequence insert comprising a nucleotide sequence encoding a RAG1 polypeptide fragment is introduced into a genome at any of the sites recited in Table 8 above. In some embodiments, the nucleotide sequence insert is introduced between chr 11:36574109 and 36574110 or between chr 11:36573910 and 36573911. In some embodiments, the nucleotide sequence insert is introduced between chr 11:36573892 and 36573893 or between chr 11:36573878 and 36573879.

When an exon 2 RAG1 gene targeting strategy is used, the nucleotide sequence insert may replace any of the regions recited in Table 9 below. In other words, the genome of the present invention may comprise the nucleotide sequence insert replacing any of the regions recited in Table 9.

TABLE 9
Exemplary insertion sites in RAG1 exon 2 (targeting strategy)

| Guide | Exemplary region to replace |
|---|---|
| g1 M5 ex2 | chr 11: 36574367 to 36574370 |
| g2 M5 ex2 | chr 11: 36574366 to 36574369 |
| g3 M5 ex2 | chr 11: 36574393 to 36574396 |
| g4 M4 ex2 | chr 11: 36574293 to 36574296 |
| g5 M3 ex2 | chr 11: 36574108 to 36574111 |
| g6 M2 ex2 | chr 11: 36573909 to 36573912 |
| g7 exon2 M2/3 | chr 11: 36573877 to 36573880 |
| g8 exon2 M2/3 | chr 11: 36573958 to 36573961 |
| g9 exon2 M2/3 | chr 11: 36573956 to 36573959 |
| g10 exon2 M2/3 | chr 11: 36573878 to 36573881 |
| g11 exon2 M2/3 | chr 11: 36573891 to 36573894 |
| g12 exon2 M2/3 | chr 11: 36573954 to 36573957 |
| g13 exon2 M2/3 | chr 11: 36573877 to 36573880 |
| g14 exon2 M5 | chr 11: 36574405 to 36574408 |

In some embodiments, the nucleotide sequence insert replaces chr 11:36574108 to 36574111 or chr 11:36573909 to 36573912. In some embodiments, the nucleotide sequence insert replaces chr 11:36573891 to 36573894 or chr 11:36573877 to 36573880.

In some embodiments, the genome of the present invention comprises a nucleotide sequence comprising a nucleotide sequence encoding a RAG1 polypeptide fragment, which replaces chr 11:36574108 to 36574111 or chr 11:36573909 to 36573912. In some embodiments, the genome of the present invention comprises a nucleotide sequence comprising a nucleotide sequence encoding a RAG1 polypeptide fragment, which replaces chr 11:36573891 to 36573894 or chr 11:36573877 to 36573880.

When an exon 2 RAG1 gene replacement strategy is used, the nucleotide sequence insert may replace any of the regions recited in Table 10 below. In other words, the genome of the present invention may comprise the nucleotide sequence insert replacing any of the regions recited in Table 10.

TABLE 10
Exemplary insertion sites in RAG1
exon 2 (replacement strategy)

| Guide | Exemplary region to replace |
|---|---|
| g1 M5 ex2 | chr 11: 36574367 to about 36576436 |
| g2 M5 ex2 | chr 11: 36574366 to about 36576436 |
| g3 M5 ex2 | chr 11: 36574393 to about 36576436 |
| g4 M4 ex2 | chr 11: 36574293 to about 36576436 |
| g5 M3 ex2 | chr 11: 36574108 to about 36576436 |
| g6 M2 ex2 | chr 11: 36573909 to about 36576436 |
| g7 exon2 M2/3 | chr 11: 36573877 to about 36576436 |
| g8 exon2 M2/3 | chr 11: 36573958 to about 36576436 |
| g9 exon2 M2/3 | chr 11: 36573956 to about 36576436 |
| g10 exon2 M2/3 | chr 11: 36573878 to about 36576436 |
| g11 exon2 M2/3 | chr 11: 36573891 to about 36576436 |
| g12 exon2 M2/3 | chr 11: 36573954 to about 36576436 |
| g13 exon2 M2/3 | chr 11: 36573877 to about 36576436 |
| g14 exon2 M5 | chr 11: 36574405 to about 36576436 |

In Table 10, “about chr 11:36576436” may refer to the end of the exon 2 CDS region or the start of the 3′UTR. Suitably, “about chr 11:36576436” may refer to chr 11:36576436±1000, chr 11:36576436±500, chr 11:36576436±400, chr 11:36576436±300, chr 11:36576436±200, chr 11:36576436±100, chr 11:36576436±50, chr 11:36576436±40, chr 11:36576436±30, chr 11:36576436±20, chr 11:36576436±10, chr 11:36576436±5, chr 11:36576436±4, chr 11:36576436±3, chr 11:36576436±2, chr 11:36576436±1, or chr 11:3657643.

In some embodiments, the nucleotide sequence insert replaces chr 11:36574108 to about 36576436 or chr 11:36573909 to about 36576436. In some embodiments, the nucleotide sequence insert replaces chr 11:36573891 to about 36576436 or chr 11:36573877 to about 36576436.

In some embodiments, the genome of the present invention comprises a nucleotide sequence comprising a nucleotide sequence encoding a RAG1 polypeptide fragment, which replaces chr 11:36574108 to about 36576436 or chr 11:36573909 to about 36576436. In some embodiments, the genome of the present invention comprises a nucleotide sequence comprising a nucleotide sequence encoding a RAG1 polypeptide fragment, which replaces chr 11:36573891 to about 36576436 or chr 11:36573877 to about 36576436.

### Intron 1 Strategies

In some embodiments, a DSB is introduced into the RAG1 intron 1 or the start of the exon 2 (e.g. the first 200 bp of the RAG1 exon 2), for example in the intron 1 strategies discussed above. For example, a DSB may be introduced at any of the sites recited in Table 11 below.

TABLE 11
Exemplary DSB sites in RAG1 intron 1 or
RAG1 exon 2 (for intron 1 strategies)

| Guide | Exemplary DSB site |
|---|---|
| 9 | between chr 11: 36569296 and 36569297 |
| 1 | between chr 11: 36573791 and 36573792 |
| 2 | between chr 11: 36573642 and 36573643 |
| 3 | between chr 11: 36573352 and 36573353 |
| 4 | between chr 11: 36569081 and 36569082 |
| 5 | between chr 11: 36572473 and 36572474 |
| 6 | between chr 11: 36571459 and 36571460 |
| 7 | between chr 11: 36571367 and 36571368 |
| 8 | between chr 11: 36572860 and 36572861 |
| 10 | between chr 11: 36571458 and 36571459 |
| 11 | between chr 11: 36569352 and 36569353 |
| 12 | between chr 11: 36572376 and 36572377 |

The nucleotide sequence insert may be introduced into a genome at any of the sites recited in Table 11 above. In other words, the genome of the present invention may comprise the nucleotide sequence insert at any of the sites recited in Table 11 above.

In some embodiments, the nucleotide sequence insert is introduced:
(i) between chr 11:36569296 and 36569297;
(ii) between chr 11:36573352 and 36573353; or
(iii) between chr 11:36571367 and 36571368.

In some embodiments, the nucleotide sequence insert is introduced between chr 11:36569296 and 36569297.

In some embodiments, the genome of the present invention comprises a nucleotide sequence comprising a splice acceptor sequence and a nucleotide sequence encoding a RAG1 polypeptide or a RAG1 polypeptide fragment, which is introduced:
(i) between chr 11:36569296 and 36569297;
(ii) between chr 11:36573352 and 36573353; or
(iii) between chr 11:36571367 and 36571368.

In some embodiments, the genome of the present invention comprises a nucleotide sequence comprising a splice acceptor sequence and a nucleotide sequence encoding a RAG1 polypeptide or a RAG1 polypeptide fragment, which is introduced between chr 11:36569296 and 36569297.

When an intron 1 RAG1 gene replacement strategy is used, the nucleotide sequence insert may replace any of the regions recited in Table 12 below. In other words, the genome of the present invention may comprise the nucleotide sequence insert replacing any of the regions recited in Table 10.

TABLE 12
Exemplary insertion sites in RAG1
exon 2 (replacement strategy)

| Guide | Exemplary region to replace |
|---|---|
| 9 | chr 11: 36569295 to about 36576436 |
| 1 | chr 11: 36573790 to about 36576436 |
| 2 | chr 11: 36573641 to about 36576436 |
| 3 | chr 11: 36573351 to about 36576436 |
| 4 | chr 11: 36569080 to about 36576436 |
| 5 | chr 11: 36572472 to about 36576436 |
| 6 | chr 11: 36571458 to about 36576436 |
| 7 | chr 11: 36571366 to about 36576436 |
| 8 | chr 11: 36572859 to about 36576436 |
| 10 | chr 11: 36571457 to about 36576436 |
| 11 | chr 11: 36569351 to about 36576436 |
| 12 | chr 11: 36572375 to about 36576436 |

In Table 10, “about chr 11:36576436” may refer to the C-terminal region of the exon 2 CDS region or the start of the 3′UTR.

In some embodiments, the nucleotide sequence insert replaces:
(i) chr 11:36569295 to about 36576436;
(ii) chr 11:36573351 to about 36576436; or
(iii) chr 11:36571366 to about 36576436.

In some embodiments, the nucleotide sequence insert replaces chr 11:36569295 to about 36576436.

In some embodiments, the genome of the present invention comprises a nucleotide sequence comprising splice acceptor sequence and a nucleotide sequence encoding a RAG1 polypeptide or a RAG1 polypeptide fragment, which replaces:
(i) chr 11:36569295 to about 36576436;
(ii) chr 11:36573351 to about 36576436; or
(iii) chr 11:36571366 to about 36576436.

In some embodiments, the genome of the present invention comprises a nucleotide sequence comprising splice acceptor sequence and a nucleotide sequence encoding a RAG1 polypeptide or a RAG1 polypeptide fragment, which replaces chr 11:36569295 to about 36576436.

Splice Acceptor and Donor Sequences RNA splicing is a form of RNA processing in which a newly made precursor messenger RNA (pre-mRNA) transcript is transformed into a mature messenger RNA (mRNA). During splicing, introns (non-coding regions) are removed and exons (coding regions) are joined together.

Within introns, a donor site (5′ end of the intron), a branch site (near the 3′ end of the intron) and an acceptor site (3′ end of the intron) are required for splicing. The splice donor site includes an almost invariant sequence GU at the 5′ end of the intron, within a larger, less highly conserved region. The splice acceptor site at the 3′ end of the intron terminates the intron with an almost invariant AG sequence. Upstream (5′-ward) from the AG there is a region high in pyrimidines (C and U), or polypyrimidine tract. Further upstream from the polypyrimidine tract is the branchpoint.

A “splice acceptor sequence” is a nucleotide sequence which can function as an acceptor site at the 3′ end of the intron. Consensus sequences and frequencies of human splice site regions are described in Ma, S. L., et al., 2015. PLoS One, 10(6), p.e0130729.

Suitably, a splice acceptor sequence may comprise the nucleotide sequence (Y)ₙNYAG, where n is 10-20, or a variant with at least 90% or at least 95% sequence identity. Suitably, a splice acceptor sequence may comprise the sequence (Y)ₙNCAG, where n is 10-20, or a variant with at least 90% or at least 95% sequence identity.

In some embodiments of the invention, a splice acceptor sequence comprises or consists of a nucleotide sequence which is at least 70% identical to SEQ ID NO: 95 or a fragment thereof. Suitably, a splice acceptor sequence comprises or consists of a nucleotide sequence which is at least 80%, at least 85%, at least 90%, at least 95%, at least 98% or at least 99% identical to SEQ ID NO: 95 or a fragment thereof.

In some embodiments of the invention, a splice acceptor sequence comprises or consists of the nucleotide sequence SEQ ID NO: 95 or a fragment thereof.
Exemplary splice acceptor sequence (SEQ ID NO: 95) ctgacctcttctcttcctcccacag

In some embodiments of the invention, the polynucleotide of the invention does not comprise a splice acceptor sequence (e.g. in exon 2 strategies).

The polynucleotide of the invention may comprise a splice donor sequence. The genome may comprise a splice donor sequence in the RAG1 intron 1. Suitably, the splice donor sequence nucleotide sequence is 3′ of the nucleotide sequence encoding a RAG1 polypeptide or a RAG1 polypeptide fragment. The splice donor sequence may be used to provide an mRNA comprising a RAG1 polypeptide.

A “splice donor sequence” is a nucleotide sequence which can function as a donor site at the 5′ end of the intron. Consensus sequences and frequencies of human splice site regions are describe in Ma, S. L., et al., 2015. PLoS One, 10(6), p.e0130729.

In some embodiments of the invention, the splice donor sequence comprises or consists of a nucleotide sequence which is at least 85% identical to SEQ ID NO: 96 or a fragment thereof. In some embodiments of the invention, the splice donor sequence comprises or consists of the nucleotide sequence SEQ ID NO: 96 or a fragment thereof.
Exemplary splice donor sequence (SEQ ID NO: 96) aggtaagt

In some embodiments of the invention, the polynucleotide of the invention does not comprise a splice donor sequence.

### Regulatory Elements

The polynucleotide of the invention may comprise one or more regulatory elements which may act pre- or post-transcriptionally. Suitably, the nucleotide sequence encoding a RAG1 polypeptide or a RAG1 polypeptide fragment is operably linked to one or more regulatory elements which may act pre- or post-transcriptionally. The one or more regulatory elements may facilitate expression of a RAG1 polypeptide in the cells of the invention.

A “regulatory element” is any nucleotide sequence which facilitates expression of a polypeptide, e.g. acts to increase expression of a transcript or to enhance mRNA stability. Suitable regulatory elements include for example promoters, enhancer elements, post-transcriptional regulatory elements and polyadenylation sites.

In preferred embodiments, the polynucleotide of the invention does not comprise a regulatory element. Endogenous regulatory elements may be sufficient to drive expression of the RAG1 polypeptide following the introduction of the nucleotide sequence insert.

### Polyadenylation Sequence

In preferred embodiments, the polynucleotide of the invention does not comprise a polyadenylation sequence.

In some embodiments, the polynucleotide of the invention may comprise a polyadenylation sequence. Suitably, the nucleotide sequence encoding a RAG1 polypeptide or a RAG1 polypeptide fragment is operably linked to a polyadenylation sequence. The polyadenylation sequence may improve gene expression.

Suitable polyadenylation sequences will be well known to those of skill in the art. Suitable polyadenylation sequences include a bovine growth hormone (BGH) polyadenylation sequence or an early SV40 polyadenylation signal. In some embodiments of the invention, the polyadenylation sequence is a BGH polyadenylation sequence.

In some embodiments of the invention, the polyadenylation sequence comprises or consists of a nucleotide sequence which is at least 70% identical to SEQ ID NO: 97, 98 or 99 or a fragment thereof. Suitably, the polyadenylation sequence comprises or consists of a nucleotide sequence which is at least 80%, at least 85%, at least 90%, at least 95%, at least 98% or at least 99% identical to SEQ ID NO: 97, 98 or 99 or a fragment thereof.

In some embodiments of the invention, the polyadenylation sequence comprises or consists of the nucleotide sequence SEQ ID NO: 97, 98 or 99 or a fragment thereof.

Exemplary BGH polyadenylation sequence
(SEQ ID NO: 97)
Gctgtgccttctagttgccagccatctgttgtttgcccctcccccgtgccttccttgaccctggaaggtgccactcccactgt
cctttcctaataaaatgaggaaattgcatcgcattgtctgagtaggtgtcattctattctggggggtggggtggggcagga
cagcaagggggaggattgggaagacaatagcaggcatgctggggatgcggtgggctctatgg
Exemplary BGH polyadenylation sequence
(SEQ ID NO: 98)
Actgtgccttctagttgccagccatctgttgtttgcccctcccccgtgccttccttgaccctggaaggtgccactcccactgc
cctttcctaataaaatgaggaaattgcatcgcattgtctgagtaggtgtcattctattctggggggtggggtggggcagga
cagcaagggggaggattgggaagacaatagcaggcatgctggggatgcggtgggctctatgg
Exemplary BGH polyadenylation sequence
(SEQ ID NO: 99)
ctgtgccttctagttgccagccatctgttgtttgcccctcccccgtgccttccttgaccctggaaggtgccactcccactgtc
ctttcctaataaaatgaggaaattgcatcgcattgtctgagtaggtgtcattctattctggggggtggggtggggcaggac
agcaagggggaggattgggaagacaatagcaggcatgctggggatgcggtgggctctatgg

### Kozak Sequence

In preferred embodiments, the polynucleotide of the invention does not comprise a Kozak sequence.

In some embodiments, the polynucleotide of the invention may comprise a Kozak sequence. Suitably, the nucleotide sequence encoding a RAG1 polypeptide or a RAG1 polypeptide fragment is operably linked to a Kozak sequence. A Kozak sequence may be inserted before the start codon of the RAG1 polypeptide or RAG1 polypeptide fragment to improve the initiation of translation.

Suitable Kozak sequences will be well known to those of skill in the art.

In some embodiments of the invention, the Kozak sequence comprises or consists of a nucleotide sequence which is at least 70% identical to SEQ ID NO: 100 or a fragment thereof. Suitably, the Kozak sequence comprises or consists of a nucleotide sequence which is at least 80%, or at least 90% identical to SEQ ID NO: 100 or a fragment thereof.

In some embodiments of the invention, the Kozak sequence comprises or consists of the nucleotide sequence SEQ ID NO: 100 or a fragment thereof.

Exemplary Kozak sequence
(SEQ ID NO: 100)
gccgccaccatg

### Post-Transcriptional Regulatory Elements

In preferred embodiments, the polynucleotide of the invention does not comprise a post-transcriptional regulatory element.

In some other embodiments, the polynucleotide of the invention may comprise a post-transcriptional regulatory element. Suitably, the nucleotide sequence encoding a RAG1 polypeptide or a RAG1 polypeptide fragment is operably linked to a post-transcriptional regulatory element. The post-transcriptional regulatory element may improve gene expression.

Suitable post-transcriptional regulatory elements will be well known to those of skill in the art.

The polynucleotide of the invention may comprise a Woodchuck Hepatitis Virus Post-transcriptional Regulatory Element (WPRE). Suitably, the nucleotide sequence encoding a RAG1 polypeptide or a RAG1 polypeptide fragment is operably linked to a WPRE.

In some embodiments of the invention, the WPRE comprises or consists of a nucleotide sequence which is at least 70% identical to SEQ ID NO: 101 or a fragment thereof. Suitably, the WPRE comprises or consists of a nucleotide sequence which is at least 80%, or at least 90% identical to SEQ ID NO: 101 or a fragment thereof.

In some embodiments of the invention, the WPRE comprises or consists of the nucleotide sequence SEQ ID NO: 101 or a fragment thereof.

Exemplary WPRE
(SEQ ID NO: 101)
aatcaacctctggattacaaaatttgtgaaagattgactggtattcttaactatgttgctccttttacgctatgtggatacgct
gctttaatgcctttgtatcatgctattgcttcccgtatggctttcattttctcctccttgtataaatcctggttgctgtctctttatgag
gagttgtggcccgttgtcaggcaacgtggcgtggtgtgcactgtgtttgctgacgcaacccccactggttggggcattgc
caccacctgtcagctcctttccgggactttcgctttccccctccctattgccacggcggaactcatcgccgcctgccttgcc
cgctgctggacaggggctcggctgttgggcactgacaattccgtggtgttgtcggggaaatcatcgtcctttccttggctg
ctcgcctgtgttgccacctggattctgcgcgggacgtccttctgctacgtcccttcggccctcaatccageggaccttcctt
cccgcggcctgctgccggctctgcggcctcttccgcgtcttcgccttcgccctcagacgagtcggatctccctttgggcc
gcctccccgcctg

In some embodiments of the invention, the RAG1 polypeptide or the RAG1 polypeptide fragment is not operably linked to a post-transcriptional regulatory element. In some embodiments of the invention, the RAG1 polypeptide or the RAG1 polypeptide fragment is not operably linked to a WPRE.

### Endoqenous 3′UTR

In preferred embodiments, the polynucleotide of the invention does not comprise an endogenous RAG1 3′ UTR.

In some other embodiments, the polynucleotide of the invention may comprise an endogenous RAG1 3′UTR. Suitably, the nucleotide sequence encoding a RAG1 polypeptide or a RAG1 polypeptide fragment is operably linked to an endogenous RAG1 3′UTR.

In some embodiments of the invention, the RAG1 3′UTR comprises or consists of a nucleotide sequence which is at least 70% identical to SEQ ID NO: 102 or a fragment thereof. Suitably, the RAG1 3′UTR comprises or consists of a nucleotide sequence which is at least 80%, or at least 90% identical to SEQ ID NO: 102 or a fragment thereof.

In some embodiments of the invention, the RAG1 3′UTR comprises or consists of the nucleotide sequence SEQ ID NO: 102 or a fragment thereof.

Exemplary RAG1 3′UTR
(SEQ ID NO: 102)
gtagggcaaccacttatgagttggtttttgcaattgagtttccctctgggttgcattgagggcttctcctagcaccctttactg
ctgtgtatggggcttcaccatccaagaggtggtaggttggagtaagatgctacagatgctctcaagtcaggaatagaa
actgatgagctgattgcttgaggcttttagtgagttccgaaaagcaacaggaaaaatcagttatctgaaagctcagtaa
ctcagaacaggagtaactgcaggggaccagagatgagcaaagatctgtgtgtgttggggagctgtcatgtaaatcaa
agccaaggttgtcaaagaacagccagtgaggccaggaaagaaattggtcttgtggttttcatttttttcccccttgattgat
tatattttgtattgagatatgataagtgccttctatttcatttttgaataattcttcatttttataattttacatatcttggcttgctatat
aagattcaaaagagctttttaaatttttctaataatatcttacatttgtacagcatgatgacctttacaaagtgctctcaatgc
atttacccattcgttatataaatatgttacatcaggacaactttgagaaaatcagtccttttttatgtttaaattatgtatctattgt
aaccttcagagtttaggaggtcatctgctgtcatggatttttcaataatgaatttagaatacacctgttagctacagttagtta
ttaaatcttctgataatatatgtttacttagctatcagaagccaagtatgattctttatttttactttttcatttcaagaaatttagag
tttccaaatttagagcttctgcatacagtcttaaagccacagaggcttgtaaaaatataggttagcttgatgtctaaaaata
tatttcatgtcttactgaaacattttgccagactttctccaaatgaaacctgaatcaatttttctaaatctaggtttcatagagtc
ctctcctctgcaatgtgttattctttctataatgatcagtttactttcagtggattcagaattgtgtagcaggataaccttgtatttt
tccatccgctaagtttagatggagtccaaacgcagtacagcagaagagttaacatttacacagtgctttttaccactgtg
gaatgttttcacactcatttttccttacaacaattctgaggagtaggtgttgttattatctccatttgatgggggtttaaatgattt
gctcaaagtcatttaggggtaataaatacttggcttggaaatttaacacagtccttttgtctccaaagcccttcttctttccac
cacaaattaatcactatgtttataaggtagtatcagaatttttttaggattcacaactaatcactatagcacatgaccttggg
attacatttttatggggcaggggtaagcaagtttttaaatcatttgtgtgctctggctcttttgatagaagaaagcaacacaa
aagctccaaagggccccctaaccctcttgtggctccagttatttggaaactatgatctgcatccttaggaatctgggatttg
ccagttgctggcaatgtagagcaggcatggaattttatatgctagtgagtcataatgatatgttagtgttaattagttttttctt
cctttgattttattggccataattgctactcttcatacacagtatatcaaagagcttgataatttagttgtcaaaagtgcatcg
gcgacattatctttaattgtatgtatttggtgcttcttcagggattgaactcagtatctttcattaaaaaacacagcagttttcct
tgctttttatatgcagaatatcaaagtcatttctaatttagttgtcaaaaacatatacatattttaacattagtttttttgaaaactc
ttggttttgtttttttggaaatgagtgggccactaagccacactttcccttcatcctgcttaatccttccagcatgtctctgcact
aataaacagctaaattcacataatcatcctatttactgaagcatggtcatgctggtttatagattttttacccatttctactctttt
tctctattggtggcactgtaaatactttccagtattaaattatccttttctaacactgtaggaactattttgaatgcatgtgacta
agagcatgatttatagcacaacctttccaataatcccttaatcagatcacattttgataaaccctgggaacatctggctgc
aggaatttcaatatgtagaaacgctgcctatggttttttgcccttactgttgagactgcaatatcctagaccctagttttatact
agagttttatttttagcaatgcctattgcaagtgcaattatatactccagggaaattcaccacactgaatcgagcatttgtgt
gtgtatgtgtgaagtatatactgggacttcagaagtgcaatgtatttttctcctgtgaaacctgaatctacaagttttcctgcc
aagccactcaggtgcattgcagggaccagtgataatggctgatgaaaattgatgattggtcagtgaggtcaaaagga
gccttgggattaataaacatgcactgagaagcaagaggaggagaaaaagatgtctttttcttccaggtgaactggaatt
tagttttgcctcagatttttttcccacaagatacagaagaagataaagatttttttggttgagagtgtgggtcttgcattacatc
aaacagagttcaaattccacacagataagaggcaggatatataagcgccagtggtagttgggaggaataaaccatt
atttggatgcaggtggtttttgattgcaaatatgtgtgtgtcttcagtgattgtatgacagatgatgtattcttttgatgttaaaag
attttaagtaagagtagatacattgtacccattttacattttcttattttaactacagtaatctacataaatatacctcagaaat
catttttggtgattattttttgttttgtagaattgcacttcagtttattttcttacaaataaccttacattttgtttaatggcttccaaga
gccttttttttttttgtatttcagagaaaattcaggtaccaggatgcaatggatttatttgattcaggggacctgtgtttccatgtc
aaatgttttcaaataaaatgaaatatgagtttcaatactttttatattttaatatttccattcattaatattatggttattgtcagca
attttatgtttgaatatttgaaataaaagtttaagatttgaaaatggtatgtattataatttctattcaaatattaataataatattg
agtgcagcatt

### Further Coding Sequences

The polynucleotide of the invention may comprise a further coding sequence. The polynucleotide of the invention may comprise an internal ribosome entry site sequence (IRES). The IRES may increase or allow expression of the further coding sequence. The IRES may be operably linked to the further coding sequence.

In some embodiments of the invention, the IRES comprises or consists of a nucleotide sequence which is at least 70% identical to SEQ ID NO: 103 or a fragment thereof. Suitably, the IRES comprises or consists of a nucleotide sequence which is at least 80%, or at least 90% identical to SEQ ID NO: 103 or a fragment thereof.

In some embodiments of the invention, the IRES comprises or consists of the nucleotide sequence SEQ ID NO: 103 or a fragment thereof.

Exemplary IRES
(SEQ ID NO: 103)
gaattaactcgaggaattccgCccctctccctcccccccccctaacgttactggccgaagccgcttggaataaggccg
gtgtgcgtttgtctatatgttattttccaccatattgccgtcttttggcaatgtgagggcccggaaacctggccctgtcttcttg
acgagcattctaggggtctttcccctctcgccaaaggaatgcaaggtctgttgaatgtcgtgaaggaagcagttcctctg
gaagcttcttgaagacaaacaacgtctgtagcgaccctttgcaggcagcggaaccccccacctggcgacaggtgcc
tctgcggccaaaagccaacgtgtataagatacacctgcaaaggggcacaaccccagtgccacgttgtgagttggat
agttgtggaaagagtcaaatggctctcctcaagcgtattcaacaaggggctgaaggatgcccagaaggtaccccatt
gtatgggatctgatctggggcctcggtgcacatgctttacatgtgtttagtcgaggttaaaaaacgtctaggccccccga
accacggggacgtggttttcctttgaaaaacacgatgataatatggccacaacc

The further coding sequence may encode a selector, for example a NGFR receptor, e.g. a low affinity NGFR, such as a C-terminal truncated low affinity NGFR. The selector may be used for enrichment of cells.

In some embodiments of the invention, the NGFR-encoding sequence comprises or consists of a nucleotide sequence which is at least 70% identical to SEQ ID NO: 104 or a fragment thereof. Suitably, the NGFR-encoding sequence comprises or consists of a nucleotide sequence which is at least 80%, or at least 90% identical to SEQ ID NO: 104 or a fragment thereof.

In some embodiments of the invention, the NGFR-encoding sequence comprises or consists of the nucleotide sequence SEQ ID NO: 104 or a fragment thereof.

Exemplary NGFR-encoding sequence
(SEQ ID NO: 104)
atgggagctggtgctaccggcagagctatggatggacctagactgctgctcctgctgctgctcggagtttctcttggcgg
agccaaagaggcctgtcctaccggcctgtatacacactctggcgagtgctgcaaggcctgcaatcttggagaaggcg
tggcacagccttgcggcgctaatcagacagtgtgcgagccttgcctggacagcgtgacctttagcgacgtggtgtctgc
caccgagccatgcaagccttgtaccgagtgtgtgggcctgcagagcatgtctgccccttgtgtggaagccgacgatgc
cgtgtgtagatgcgcctacggctactaccaggacgagacaacaggcagatgcgaggcctgtagagtgtgtgaagcc
ggctctggactggtgttcagctgccaagacaagcagaacaccgtgtgcgaggaatgccccgatggcacctatagcg
acgaggccaaccatgtagatccctgcctgccttgtactgtgtgcgaagataccgagcggcagctgcgcgagtgtaca
agatgggctgatgccgagtgcgaagagatccccggcagatggatcaccagaagcacacctccagagggcagcg
atagcacagccccttctacacaagagcccgaggctcctcctgagcaggatctgattgcctctacagtggccggcgtgg
tcacaacagtgatgggatcttctcagcccgtggtcaccagaggcaccaccgacaatctgatccccgtgtactgtagca
tcctggccgccgtggttgtgggactcgtggcctatatcgccttcaagcggtggaaccggggcatcctgtaa

The further coding sequence may encode a destabilisation domain, for example a peptide sequence rich in proline (P), glutamic acid (E), serine (S), and threonine (T) (PEST). Endogenous RAG1 protein may be destabilized by the destabilisation domain, e.g. PEST signal peptide via proteasome degradation.

In some embodiments of the invention, the PEST-encoding sequence comprises or consists of a nucleotide sequence which is at least 70% identical to SEQ ID NO: 105 or a fragment thereof. Suitably, the PEST-encoding sequence comprises or consists of a nucleotide sequence which is at least 80%, or at least 90% identical to SEQ ID NO: 105 or a fragment thereof.

In some embodiments of the invention, the PEST-encoding sequence comprises or consists of the nucleotide sequence SEQ ID NO: 105 or a fragment thereof.

Exemplary PEST-encoding sequence
(SEQ ID NO: 105)
atgaggaccgaggcccccgagggcaccgagagcgagatggagaccccca
gcgccatcaacggcaaccccagctggcac

### Promoters and Enhancers

In preferred embodiments, the polynucleotide of the invention does not comprise a promoter or an enhancer element. Transcription of a nucleotide sequence encoding a RAG1 polypeptide may be driven by an endogenous promoter. For example, if the polynucleotide of the present invention is inserted into the RAG1 intron 1 or exon 2, transcription of a nucleotide sequence encoding a RAG1 polypeptide may be driven by the endogenous RAG1 promoter.

In some other embodiments, the nucleotide sequence encoding a RAG1 polypeptide or a RAG1 polypeptide fragment is operably linked to a promoter and/or enhancer element.

A “promoter” is a region of DNA that leads to initiation of transcription of a gene. Promoters are located near the transcription start sites of genes, upstream on the DNA (towards the 5′ region of the sense strand). Any suitable promoter may be used, the selection of which may be readily made by the skilled person.

An “enhancer” is a region of DNA that can be bound by proteins (activators) to increase the likelihood that transcription of a particular gene will occur. Enhancers are cis-acting. They can be located up to 1 Mbp (1,000,000 bp) away from the gene, upstream or downstream from the start site. Any suitable enhancer may be used, the selection of which may be readily made by the skilled person.

### Exemplary Polynucleotides and Genomes

In some embodiments, the polynucleotide of the invention comprises, essentially consists of, or consists of from 5′ to 3′: a first homology region, a nucleotide sequence encoding a RAG1 polypeptide or a RAG1 polypeptide fragment, and a second homology region.

In some embodiments, the polynucleotide of the invention comprises, essentially consists of, or consists of from 5′ to 3′: a first homology region, a nucleotide sequence a RAG1 polypeptide fragment, and a second homology region.

In some embodiments, the polynucleotide of the invention comprises, essentially consists of, or consists of from 5′ to 3′: a first homology region, a splice acceptor sequence, a nucleotide sequence encoding a RAG1 polypeptide or a RAG1 polypeptide fragment, and a second homology region.

In some embodiments, the polynucleotide of the invention comprises, essentially consists of, or consists of from 5′ to 3′: a first homology region, a splice acceptor sequence, a nucleotide sequence encoding a RAG1 polypeptide, and a second homology region.

In some embodiments, the polynucleotide of the invention comprises or consists of a nucleotide sequence that has at least 70%, at least 75%, at least 80%, at least 85%, at least 90%, at least 95%, at least 98%, or at least 99% identity to any of SEQ ID NOs: 106-116 or 160-163.

In some embodiments, the polynucleotide of the invention comprises or consists of the nucleotide sequence any of SEQ ID NOs: 106-116 or 160-163.

In some embodiments, the genome of the invention comprises a nucleotide sequence that has at least 70%, at least 75%, at least 80%, at least 85%, at least 90%, at least 95%, at least 98%, or at least 99% identity to any of SEQ ID NOs: 106-116 or 160-163.

In some embodiments, the genome of the invention comprises the nucleotide sequence of any of SEQ ID NOs: 106-116 or 160-163.

Exemplary polynucleotide specific for “g5 M3 ex2 RAG1” gRNA for the exon 2 RAG1
gene targeting strategy
(SEQ ID NO: 106)
gatccatcaagccaaccttcgacatctctgccgcatctgtgggaattcttttagagctgatgagcacaacaggagatatc
cagtccatggtcctgtggatggtaaaaccctaggccttttacgaaagaaggaaaagagagctacttcctggccggac
ctcattgccaaggttttccggatcgatgtgaaggcagatgttgactcgatccaccccactgagttctgccataactgctgg
agcatcatgcacaggaagtttagcagtgccccatgtgaggtttacttcccgaggaacgtgaccatggagtggcacccc
cacacaccatcctgtgacatctgcaacactgcccgtcggggactcaagaggaagagtcttcagccaaacttgcagct
cagcaaaaaactcaaaactgtgcttgaccaagcaagacaagcccgtcagcgcaagagaagagctcaggcaagg
atcagcagcaaggatgtcatgaagaagatcgcaaactgcagcaagatccacctgagcaccaaactgctggccgtg
gacttccctgagcacttcgtgaagtccatcagctgccagatctgcgagcacatcctggccgatcctgtggaaacaaac
tgcaagcacgtgttctgcagagtgtgcatcctgcggtgcctgaaagtgatgggcagctactgcccctcctgcagatacc
cttgcttccccaccgatctggaaagccctgtgaagtccttcctgagcgtgctgaacagcctgatggtcaagtgccccgc
caaagaatgcaacgaggaagtgtccctggaaaagtacaaccaccacatcagcagccacaaagagtccaaagaa
atcttcgtgcacatcaacaaaggcggcagaccccggcagcatctgctgtctcttacaagacgggcccagaagcacc
ggctgagagaactgaagctgcaagtgaaggcctttgccgacaaagaggaaggcggcgacgtcaagagcgtgtgc
atgaccctgtttctgctggccctgagagcccggaatgagcatagacaggccgatgagctggaagccatcatgcaag
gcaaaggcagcggactgcagcctgctgtgtgtctggctatcagagtgaacaccttcctgtcctgcagccagtaccaca
agatgtaccggaccgtgaaggccattaccggcagacagatcttccagcctctgcacgccctgagaaacgccgaga
aagttctgctgcctggctaccaccacttcgagtggcagcctccactgaagaacgtgtccagcagcaccgacgtgggc
atcatcgatggactgagcggactgtctagcagcgtggacgactaccccgtggacacaatcgccaagcggttcagata
cgacagcgccctggtgtctgccctgatggacatggaagaggacatcctggaaggcatgcggagccaggacctgga
cgattacctgaacggccctttcaccgtggtggtcaaagaaagctgtgacggcatgggcgacgtgtccgagaaacac
ggatctggacctgtggtgccagagaaggccgtgcggttcagcttcaccatcatgaagatcactatcgcccacagcag
ccagaacgtgaaagtgttcgaggaagccaagcctaacagcgagctgtgctgcaagcctctgtgtctgatgctggccg
acgagagcgatcacgagacactgaccgccattctgagccctctgatcgccgaacgggaagccatgaagtcctccg
agctgatgctcgaactcggcggcatcctgagaaccttcaagttcatcttccgcggcaccggctacgacgagaagctc
gttagagaggtggaaggcctggaagcctctggcagcgtgtacatctgcaccctgtgtgacgccaccagactggaag
ctagccagaacctggtgttccacagcatcaccagaagccacgccgaaaacctggaaagatacgaagtgtggcgga
gcaacccctaccacgagagcgtggaagaactgcgggatagagtgaagggcgtgtccgccaagcctttcatcgaga
cagtgcctagcatcgacgccctgcactgcgatattggcaacgccgccgaattctacaagatctttcagctggaaatcg
gcgaggtgtacaagaaccccaacgcctctaaagaggaacggaagcgctggcaggccacactggataagcacctg
agaaagaagatgaatctgaagcccatcatgaggatgaacggcaacttcgcccggaagctgatgaccaaagaaac
cgtggatgccgtgtgcgagctgatcccctctgaggaaagacacgaggccctgcgggaactgatggacctgtacctga
agatgaagcccgtgtggcggtctagctgtcctgccaaagagtgccctgagtctctgtgccagtacagcttcaacagcc
agagattcgccgagctgctgtccaccaagttcaagtacagatacgagggcaagatcaccaactacttccacaagac
cctggctcacgtgcccgagatcatcgagagagatggctctattggcgcctgggcctctgagggcaatgagtctggcaa
caagctgttccggcggttccgcaagatgaacgccagacagagcaagtgctacgagatggaagatgtgctgaagca
ccactggctgtacaccagcaagtacctgcagaaattcatgaacgcccacaacgccctcaagaccagcggctttacc
atgaatcctcaggccagcctgggcgaccctctgggaattgaggatagcctggaatcccaggacagcatggaattctg
ataagcagtaagatacatcttagtaccaagctccttgcagtggacttcccagagcactttgtgaaatccatctcctgcca
gatctgtgaacacattctggctgaccctgtggagaccaactgtaagcatgtcttttgccgggtctgcattctcagatgcctc
aaagtcatgggcagctattgtccctcttgccgatatccatgcttccctactgacctggagagtccagtgaagtcctttctga
gcgtcttgaa
Exemplary polynucleotide specific for “g5 M3 ex2 RAG1” gRNA for the exon 2 RAG1
gene replacement strategy with long right HA
(SEQ ID NO: 107)
aaaaccctaggccttttacgaaagaaggaaaagagagctacttcctggccggacctcattgccaaggttttccggatc
gatgtgaaggcagatgttgactcgatccaccccactgagttctgccataactgctggagcatcatgcacaggaagttta
gcagtgccccatgtgaggtttacttcccgaggaacgtgaccatggagtggcacccccacacaccatcctgtgacatct
gcaacactgcccgtcggggactcaagaggaagagtcttcagccaaacttgcagctcagcaaaaaactcaaaactgt
gcttgaccaagcaagacaagcccgtcagcgcaagagaagagctcaggcaaggatcagcagcaaggatgtcatg
aagaagatcgcaaactgcagcaagatccacctgagcaccaaactgctggccgtggacttccctgagcacttcgtga
agtccatcagctgccagatctgcgagcacatcctggccgatcctgtggaaacaaactgcaagcacgtgttctgcaga
gtgtgcatcctgcggtgcctgaaagtgatgggcagctactgcccctcctgcagatacccttgcttccccaccgatctgga
aagccctgtgaagtccttcctgagcgtgctgaacagcctgatggtcaagtgccccgccaaagaatgcaacgaggaa
gtgtccctggaaaagtacaaccaccacatcagcagccacaaagagtccaaagaaatcttcgtgcacatcaacaaa
ggcggcagaccccggcagcatctgctgtctcttacaagacgggcccagaagcaccggctgagagaactgaagctg
caagtgaaggcctttgccgacaaagaggaaggcggcgacgtcaagagcgtgtgcatgaccctgtttctgctggccct
gagagcccggaatgagcatagacaggccgatgagctggaagccatcatgcaaggcaaaggcagcggactgcag
cctgctgtgtgtctggctatcagagtgaacaccttcctgtcctgcagccagtaccacaagatgtaccggaccgtgaagg
ccattaccggcagacagatcttccagcctctgcacgccctgagaaacgccgagaaagttctgctgcctggctaccac
cacttcgagtggcagcctccactgaagaacgtgtccagcagcaccgacgtgggcatcatcgatggactgagcggac
tgtctagcagcgtggacgactaccccgtggacacaatcgccaagcggttcagatacgacagcgccctggtgtctgcc
ctgatggacatggaagaggacatcctggaaggcatgcggagccaggacctggacgattacctgaacggccctttca
ccgtggtggtcaaagaaagctgtgacggcatgggcgacgtgtccgagaaacacggatctggacctgtggtgccaga
gaaggccgtgcggttcagcttcaccatcatgaagatcactatcgcccacagcagccagaacgtgaaagtgttcgagg
aagccaagcctaacagcgagctgtgctgcaagcctctgtgtctgatgctggccgacgagagcgatcacgagacact
gaccgccattctgagccctctgatcgccgaacgggaagccatgaagtcctccgagctgatgctcgaactcggcggca
tcctgagaaccttcaagttcatcttccgcggcaccggctacgacgagaagctcgttagagaggtggaaggcctggaa
gcctctggcagcgtgtacatctgcaccctgtgtgacgccaccagactggaagctagccagaacctggtgttccacagc
atcaccagaagccacgccgaaaacctggaaagatacgaagtgtggcggagcaacccctaccacgagagcgtgg
aagaactgcgggatagagtgaagggcgtgtccgccaagcctttcatcgagacagtgcctagcatcgacgccctgca
ctgcgatattggcaacgccgccgaattctacaagatctttcagctggaaatcggcgaggtgtacaagaaccccaacg
cctctaaagaggaacggaagcgctggcaggccacactggataagcacctgagaaagaagatgaatctgaagccc
atcatgaggatgaacggcaacttcgcccggaagctgatgaccaaagaaaccgtggatgccgtgtgcgagctgatcc
cctctgaggaaagacacgaggccctgcgggaactgatggacctgtacctgaagatgaagcccgtgtggcggtctag
ctgtcctgccaaagagtgccctgagtctctgtgccagtacagcttcaacagccagagattcgccgagctgctgtccacc
aagttcaagtacagatacgagggcaagatcaccaactacttccacaagaccctggctcacgtgcccgagatcatcg
agagagatggctctattggcgcctgggcctctgagggcaatgagtctggcaacaagctgttccggcggttccgcaag
atgaacgccagacagagcaagtgctacgagatggaagatgtgctgaagcaccactggctgtacaccagcaagtac
ctgcagaaattcatgaacgcccacaacgccctcaagaccagcggctttaccatgaatcctcaggccagcctgggcg
atcctttaggcatagaggactctctggaaagccaagattcaatggaattttaagtagggcaaccacttatgagttggttttt
gcaattgagtttccctctgggttgcattgagggcttctcctagcaccctttactgctgtgtatggggcttcaccatccaagag
gtggtaggttggagtaagatgctacagatgctctcaagtcaggaatagaaactgatgagctgattgcttgaggcttttag
tgagttccgaaaagcaacaggaaaaatcagttatctgaaagctcagtaactcagaacaggagtaactgcagggga
ccagagatgagcaaagatctgtgtgtgttggggagctgtcatgtaaatcaaagccaaggttgtcaaagaacagccag
tgaggccaggaaagaaattggtcttgtggttttcatttttttcccccttgattgattatattttgtattgagatatgataagtgcctt
ctatttcatttttgaataattcttcatttttataattttacatatcttggcttgctatataagattcaaaagagctttttaaatttttctaa
taatatcttacatttgtacagcatgatgacctttacaaagtgctctcaatgcatttacccattcgttatataaatatgttacatc
aggacaactttgagaaaatcagtccttttttatgtttaaattatgtatctattgtaaccttcagagtttaggaggtcatctgctgt
catggatttttcaataatgaatttagaatacacctgttagctacagttagttattaaatcttctgataatatatgtttacttagcta
tcagaagccaagtatgattctttatttttactttttcatttcaagaaatttagagtttccaaatttagagcttctgcatacagtctt
aaagccacagaggcttgtaaaaatataggttagcttgatgtctaaaaatatatttcatgtcttactgaaacattttgccaga
ctttctccaaatgaaacctgaatcaatttttctaaatctaggtttcatagagtcctctcctctgcaatgtgttattctttctataat
gatcagtttactttcagtggattcagaattgtgtagcaggataaccttgtatttttccatccgctaagtttagatggagtccaa
acgcagtacagcagaagagttaacatttacacagtgctttttaccactgtggaatgttttcacactcatttttccttacaaca
attctgaggagtaggtgttgttattatctccatttgatgggggtttaaatgatttgctcaaagtcatttaggggtaataaatact
tggcttggaaatttaacacagtccttttgtctccaaagcccttcttctttccaccacaaattaatcactatgtttataaggtagt
atcagaatttttttaggattcacaactaatcactatagcacatgaccttgggattacatttttatggggcaggggtaagcaa
gtttttaaatcatttgtgtgctctggctcttttgatagaagaaagcaacacaaaagctccaaagggccccctaaccctctt
gtggctccagttatttggaaactatgatctgcatccttaggaatctgggatttgccagttgctggcaatgtagagcaggca
tggaattttatatgctagtgagtcataatgatatgttagtgttaattagttttttcttcctttgattttattggccataattgctactctt
catacacagtatatcaaagagcttgataatttagttgtcaaaag
Exemplary polynucleotide specific for “g5 M3 ex2 RAG1” gRNA for the exon 2 RAG1
gene replacement strategy with short right HA
(SEQ ID NO: 108)
aaaaccctaggccttttacgaaagaaggaaaagagagctacttcctggccggacctcattgccaaggttttccggatc
gatgtgaaggcagatgttgactcgatccaccccactgagttctgccataactgctggagcatcatgcacaggaagttta
gcagtgccccatgtgaggtttacttcccgaggaacgtgaccatggagtggcacccccacacaccatcctgtgacatct
gcaacactgcccgtcggggactcaagaggaagagtcttcagccaaacttgcagctcagcaaaaaactcaaaactgt
gcttgaccaagcaagacaagcccgtcagcgcaagagaagagctcaggcaaggatcagcagcaaggatgtcatg
aagaagatcgcaaactgcagcaagatccacctgagcaccaaactgctggccgtggacttccctgagcacttcgtga
agtccatcagctgccagatctgcgagcacatcctggccgatcctgtggaaacaaactgcaagcacgtgttctgcaga
gtgtgcatcctgcggtgcctgaaagtgatgggcagctactgcccctcctgcagatacccttgcttccccaccgatctgga
aagccctgtgaagtccttcctgagcgtgctgaacagcctgatggtcaagtgccccgccaaagaatgcaacgaggaa
gtgtccctggaaaagtacaaccaccacatcagcagccacaaagagtccaaagaaatcttcgtgcacatcaacaaa
ggcggcagaccccggcagcatctgctgtctcttacaagacgggcccagaagcaccggctgagagaactgaagctg
caagtgaaggcctttgccgacaaagaggaaggcggcgacgtcaagagcgtgtgcatgaccctgtttctgctggccct
gagagcccggaatgagcatagacaggccgatgagctggaagccatcatgcaaggcaaaggcagcggactgcag
cctgctgtgtgtctggctatcagagtgaacaccttcctgtcctgcagccagtaccacaagatgtaccggaccgtgaagg
ccattaccggcagacagatcttccagcctctgcacgccctgagaaacgccgagaaagttctgctgcctggctaccac
cacttcgagtggcagcctccactgaagaacgtgtccagcagcaccgacgtgggcatcatcgatggactgagcggac
tgtctagcagcgtggacgactaccccgtggacacaatcgccaagcggttcagatacgacagcgccctggtgtctgcc
ctgatggacatggaagaggacatcctggaaggcatgcggagccaggacctggacgattacctgaacggccctttca
ccgtggtggtcaaagaaagctgtgacggcatgggcgacgtgtccgagaaacacggatctggacctgtggtgccaga
gaaggccgtgcggttcagcttcaccatcatgaagatcactatcgcccacagcagccagaacgtgaaagtgttcgagg
aagccaagcctaacagcgagctgtgctgcaagcctctgtgtctgatgctggccgacgagagcgatcacgagacact
gaccgccattctgagccctctgatcgccgaacgggaagccatgaagtcctccgagctgatgctcgaactcggcggca
tcctgagaaccttcaagttcatcttccgcggcaccggctacgacgagaagctcgttagagaggtggaaggcctggaa
gcctctggcagcgtgtacatctgcaccctgtgtgacgccaccagactggaagctagccagaacctggtgttccacagc
atcaccagaagccacgccgaaaacctggaaagatacgaagtgtggcggagcaacccctaccacgagagcgtgg
aagaactgcgggatagagtgaagggcgtgtccgccaagcctttcatcgagacagtgcctagcatcgacgccctgca
ctgcgatattggcaacgccgccgaattctacaagatctttcagctggaaatcggcgaggtgtacaagaaccccaacg
cctctaaagaggaacggaagcgctggcaggccacactggataagcacctgagaaagaagatgaatctgaagccc
atcatgaggatgaacggcaacttcgcccggaagctgatgaccaaagaaaccgtggatgccgtgtgcgagctgatcc
cctctgaggaaagacacgaggccctgcgggaactgatggacctgtacctgaagatgaagcccgtgtggcggtctag
ctgtcctgccaaagagtgccctgagtctctgtgccagtacagcttcaacagccagagattcgccgagctgctgtccacc
aagttcaagtacagatacgagggcaagatcaccaactacttccacaagaccctggctcacgtgcccgagatcatcg
agagagatggctctattggcgcctgggcctctgagggcaatgagtctggcaacaagctgttccggcggttccgcaag
atgaacgccagacagagcaagtgctacgagatggaagatgtgctgaagcaccactggctgtacaccagcaagtac
ctgcagaaattcatgaacgcccacaacgccctcaagaccagcggctttaccatgaatcctcaggccagcctgggcg
atcctttaggcatagaggactctctggaaagccaagattcaatggaattttaagtagggcaaccacttatgagttggttttt
gcaattgagtttccctctgggttgcattgagggcttctcctagcaccctttactgctgtgtatggggcttcaccatccaagag
gtggtaggttggagtaagatgctacagatgctctcaagtcaggaatagaaactgatgagctg attgcttgaggcttttag
tgagttccgaaaagcaacaggaaaaatcagttatctgaaagctcagtaactcagaacaggagtaactgcagggga
ccagagatgagcaaagatctgtgtgtgttggggagctgtcatgtaaatcaaagccaaggttgtcaaagaacagccag
tgaggccaggaaagaaattggtcttgtggttttcatttttttcccccttgattgattatattttgtattgagatatgataagtgcctt
ctatttcatttttgaataattcttcatttttataattttacatatcttggcttgctatataagattcaaaagagctttttaaatttttctaa
taatatcttacatttgtacagcatgatgacctttacaaagtgctctcaatgcatttacccattcgttatataaatatgttacatc
aggacaactttgagaaaatcagtccttttttatgtttaaattatgtatctattgtaaccttcagagtttaggaggtcatctgctgt
catggatttttcaataatgaatttagaatacacctgttagctacagttagttattaaatcttctgataatatatgtttacttagcta
tcagaagccaagtatgattctttatttttactttttcatttcaagaaatttagagtttccaaatttagagct
Exemplary polynucleotide specific for “g6 M2 ex2 RAG1” gRNA for the exon 2 RAG1
gene targeting strategy
(SEQ ID NO: 109)
tgagatcctttgaaaagacacctgaagaagctcaaaaggaaaagaaggattcctttgaggggaaaccctctctgga
gcaatctccagcagtcctggacaaggctgatggtcagaagccagtcccaactcagccattgttaaaagcccacccta
agttttcaaagaaatttcacgacaacgagaaagcaagaggcaaagcgatccatcaagccaaccttcgacatctctg
ccgcatctgtgggaattcttttagagctgatgagcacaacaggagatatccagtccatggtcctgtggatggtaaaacc
ctaggccttttacgaaagaaggaaaagagagctacttcctggccggacctcattgccaaggttttccggatcgatgtga
aggcagatgttgactcgatccaccccactgagttctgccataactgctggagcatcatgcacaggaagtttagcagtgc
cccatgtgaggtttacttcccgaggaatgtcactatggaatggcaccctcacacacccagctgcgacatctgcaacac
agccagaagaggcctgaagcggaagtccctgcagcctaatctgcagctgagcaagaaactgaaaaccgtgctgg
accaggccagacaggcccggcaaagaaagagaagggcccaagccagaatcagcagcaaggacgtgatgaag
aagatcgccaactgcagcaagatccacctgagcaccaaactgctggccgtggacttccctgagcacttcgtgaagtc
catcagctgccagatctgcgagcacatcctggccgatcctgtggaaacaaactgcaagcacgtgttctgcagagtgtg
catcctgcggtgcctgaaagtgatgggcagctactgcccctcctgcagatacccttgcttccccaccgatctggaaagc
cctgtgaagtccttcctgagcgtgctgaacagcctgatggtcaagtgccccgccaaagaatgcaacgaggaagtgtc
cctggaaaagtacaaccaccacatcagcagccacaaagagtccaaagaaatcttcgtgcacatcaacaaaggcg
gcagaccccggcagcatctgctgtctcttacaagacgggcccagaagcaccggctgagagaactgaagctgcaag
tgaaggcctttgccgacaaagaggaaggcggcgacgtcaagagcgtgtgcatgaccctgtttctgctggccctgaga
gcccggaatgagcatagacaggccgatgagctggaagccatcatgcaaggcaaaggcagcggactgcagcctg
ctgtgtgtctggctatcagagtgaacaccttcctgtcctgcagccagtaccacaagatgtaccggaccgtgaaggccat
taccggcagacagatcttccagcctctgcacgccctgagaaacgccgagaaagttctgctgcctggctaccaccactt
cgagtggcagcctccactgaagaacgtgtccagcagcaccgacgtgggcatcatcgatggactgagcggactgtct
agcagcgtggacgactaccccgtggacacaatcgccaagcggttcagatacgacagcgccctggtgtctgccctga
tggacatggaagaggacatcctggaaggcatgcggagccaggacctggacgattacctgaacggccctttcaccgt
ggtggtcaaagaaagctgtgacggcatgggcgacgtgtccgagaaacacggatctggacctgtggtgccagagaa
ggccgtgcggttcagcttcaccatcatgaagatcactatcgcccacagcagccagaacgtgaaagtgttcgaggaag
ccaagcctaacagcgagctgtgctgcaagcctctgtgtctgatgctggccgacgagagcgatcacgagacactgac
cgccattctgagccctctgatcgccgaacgggaagccatgaagtcctccgagctgatgctcgaactcggcggcatcct
gagaaccttcaagttcatcttccgcggcaccggctacgacgagaagctcgttagagaggtggaaggcctggaagcc
tctggcagcgtgtacatctgcaccctgtgtgacgccaccagactggaagctagccagaacctggtgttccacagcatc
accagaagccacgccgaaaacctggaaagatacgaagtgtggcggagcaacccctaccacgagagcgtggaa
gaactgcgggatagagtgaagggcgtgtccgccaagcctttcatcgagacagtgcctagcatcgacgccctgcactg
cgatattggcaacgccgccgaattctacaagatctttcagctggaaatcggcgaggtgtacaagaaccccaacgcct
ctaaagaggaacggaagcgctggcaggccacactggataagcacctgagaaagaagatgaatctgaagcccatc
atgaggatgaacggcaacttcgcccggaagctgatgaccaaagaaaccgtggatgccgtgtgcgagctgatcccct
ctgaggaaagacacgaggccctgcgggaactgatggacctgtacctgaagatgaagcccgtgtggcggtctagctg
tcctgccaaagagtgccctgagtctctgtgccagtacagcttcaacagccagagattcgccgagctgctgtccaccaa
gttcaagtacagatacgagggcaagatcaccaactacttccacaagaccctggctcacgtgcccgagatcatcgag
agagatggctctattggcgcctgggcctctgagggcaatgagtctggcaacaagctgttccggcggttccgcaagatg
aacgccagacagagcaagtgctacgagatggaagatgtgctgaagcaccactggctgtacaccagcaagtacctg
cagaaattcatgaacgcccacaacgccctcaagaccagcggctttaccatgaatcctcaggccagcctgggcgacc
ctctgggaattgaggatagcctggaatcccaggacagcatggaattctgataagagtggcacccccacacaccatcc
tgtgacatctgcaacactgcccgtcggggactcaagaggaagagtcttcagccaaacttgcagctcagcaaaaaact
caaaactgtgcttgaccaagcaagacaagcccgtcagcgcaagagaagagctcaggcaaggatcagcagcaag
gatgtcatgaagaagatcgccaactgcagtaagatacatcttagtaccaagctccttgcagtggacttcccagagc
Exemplary polynucleotide specific for “g6 M2 ex2 RAG1” gRNA for the exon 2 RAG1
gene replacement strategy with long right HA
(SEQ ID NO: 110)
gagcacaacaggagatatccagtccatggtcctgtggatggtaaaaccctaggccttttacgaaagaaggaaaaga
gagctacttcctggccggacctcattgccaaggttttccggatcgatgtgaaggcagatgttgactcgatccaccccact
gagttctgccataactgctggagcatcatgcacaggaagtttagcagtgccccatgtgaggtttacttcccgaggaatgt
cactatggaatggcaccctcacacacccagctgcgacatctgcaacacagccagaagaggcctgaagcggaagt
ccctgcagcctaatctgcagctgagcaagaaactgaaaaccgtgctggaccaggccagacagg cccggcaaaga
aagagaagggcccaagccagaatcagcagcaaggacgtgatgaagaagatcgccaactgcagcaagatccac
ctgagcaccaaactgctggccgtggacttccctgagcacttcgtgaagtccatcagctgccagatctgcgagcacatc
ctggccgatcctgtggaaacaaactgcaagcacgtgttctgcagagtgtgcatcctgcggtgcctgaaagtgatgggc
agctactgcccctcctgcagatacccttgcttccccaccgatctggaaagccctgtgaagtccttcctgagcgtgctgaa
cagcctgatggtcaagtgccccgccaaagaatgcaacgaggaagtgtccctggaaaagtacaaccaccacatcag
cagccacaaagagtccaaagaaatcttcgtgcacatcaacaaaggcggcagaccccggcagcatctgctgtctctt
acaagacgggcccagaagcaccggctgagagaactgaagctgcaagtgaaggcctttgccgacaaagaggaag
gcggcgacgtcaagagcgtgtgcatgaccctgtttctgctggccctgagagcccggaatgagcatagacaggccgat
gagctggaagccatcatgcaaggcaaaggcagcggactgcagcctgctgtgtgtctggctatcagagtgaacacctt
cctgtcctgcagccagtaccacaagatgtaccggaccgtgaaggccattaccggcagacagatcttccagcctctgc
acgccctgagaaacgccgagaaagttctgctgcctggctaccaccacttcgagtggcagcctccactgaagaacgt
gtccagcagcaccgacgtgggcatcatcgatggactgagcggactgtctagcagcgtggacgactaccccgtggac
acaatcgccaagcggttcagatacgacagcgccctggtgtctgccctgatggacatggaagaggacatcctggaag
gcatgcggagccaggacctggacgattacctgaacggccctttcaccgtggtggtcaaagaaagctgtgacggcat
gggcgacgtgtccgagaaacacggatctggacctgtggtgccagagaaggccgtgcggttcagcttcaccatcatg
aagatcactatcgcccacagcagccagaacgtgaaagtgttcgaggaagccaagcctaacagcgagctgtgctgc
aagcctctgtgtctgatgctggccgacgagagcgatcacgagacactgaccgccattctgagccctctgatcgccga
acgggaagccatgaagtcctccgagctgatgctcgaactcggcggcatcctgagaaccttcaagttcatcttccgcgg
caccggctacgacgagaagctcgttagagaggtggaaggcctggaagcctctggcagcgtgtacatctgcaccctgt
gtgacgccaccagactggaagctagccagaacctggtgttccacagcatcaccagaagccacgccgaaaacctg
gaaagatacgaagtgtggcggagcaacccctaccacgagagcgtggaagaactgcgggatagagtgaagggcg
tgtccgccaagcctttcatcgagacagtgcctagcatcgacgccctgcactgcgatattggcaacgccgccgaattcta
caagatctttcagctggaaatcggcgaggtgtacaagaaccccaacgcctctaaagaggaacggaagcgctggca
ggccacactggataagcacctgagaaagaagatgaatctgaagcccatcatgaggatgaacggcaacttcgcccg
gaagctgatgaccaaagaaaccgtggatgccgtgtgcgagctgatcccctctgaggaaagacacgaggccctgcg
ggaactgatggacctgtacctgaagatgaagcccgtgtggcggtctagctgtcctgccaaagagtgccctgagtctct
gtgccagtacagcttcaacagccagagattcgccgagctgctgtccaccaagttcaagtacagatacgagggcaag
atcaccaactacttccacaagaccctggctcacgtgcccgagatcatcgagagagatggctctattggcgcctgggcc
tctgagggcaatgagtctggcaacaagctgttccggcggttccgcaagatgaacgccagacagagcaagtgctacg
agatggaagatgtgctgaagcaccactggctgtacaccagcaagtacctgcagaaattcatgaacgcccacaacgc
cctcaagaccagcggctttaccatgaatcctcaggccagcctgggcgatcctttaggcatagaggactctctggaaag
ccaagattcaatggaattttaagtagggcaaccacttatgagttggtttttgcaattgagtttccctctgggttgcattgagg
gcttctcctagcaccctttactgctgtgtatggggcttcaccatccaagaggtggtaggttggagtaagatgctacagatg
ctctcaagtcaggaatagaaactgatgagctgattgcttgaggcttttagtgagttccgaaaagcaacaggaaaaatc
agttatctgaaagctcagtaactcagaacaggagtaactgcaggggaccagagatgagcaaagatctgtgtgtgttg
gggagctgtcatgtaaatcaaagccaaggttgtcaaagaacagccagtgaggccaggaaagaaattggtcttgtggt
tttcatttttttcccccttgattgattatattttgtattgagatatgataagtgccttctatttcatttttgaataattcttcatttttata
attttacatatcttggcttgctatataagattcaaaagagctttttaaatttttctaataatatcttacatttgtacagcatgatgacct
ttacaaagtgctctcaatgcatttacccattcgttatataaatatgttacatcaggacaactttgagaaaatcagtcctttttta
tgtttaaattatgtatctattgtaaccttcagagtttaggaggtcatctgctgtcatggatttttcaataatgaatttagaataca
cctgttagctacagttagttattaaatcttctgataatatatgtttacttagctatcagaagccaagtatgattctttatttttacttt
ttcatttcaagaaatttagagtttccaaatttagagcttctgcatacagtcttaaagccacagaggcttgtaaaaatatagg
ttagcttgatgtctaaaaatatatttcatgtcttactgaaacattttgccagactttctccaaatgaaacctgaatcaatttttct
aaatctaggtttcatagagtcctctcctctgcaatgtgttattctttctataatgatcagtttactttcagtggattcagaattgtg
tagcaggataaccttgtatttttccatccgctaagtttagatggagtccaaacgcagtacagcagaagagttaacatttac
acagtgctttttaccactgtggaatgttttcacactcatttttccttacaacaattctgaggagtaggtgttgttattatctccattt
gatgggggtttaaatgatttgctcaaagtcatttaggggtaataaatacttggcttggaaatttaacacagtccttttgtctcc
aaagcccttcttctttccaccacaaattaatcactatgtttataaggtagtatcagaatttttttaggattcacaactaatcact
atagcacatgaccttgggattacatttttatggggcaggggtaagcaagtttttaaatcatttgtgtgctctggctcttttgata
gaagaaagcaacacaaaagctccaaagggccccctaaccctcttgtggctccagttatttggaaactatgatctgcat
ccttaggaatctgggatttgccagttgctggcaatgtagagcaggcatggaattttatatgctagtgagtcataatgatatg
ttagtgttaattagttttttcttcctttgattttattggccataattgctactcttcatacacagtatatcaaagagcttgataattta
gttgtcaaaag
Exemplary polynucleotide specific for “g6 M2 ex2 RAG1” gRNA for the exon 2 RAG1
gene replacement strategy with short right HA
(SEQ ID NO: 111)
gagcacaacaggagatatccagtccatggtcctgtggatggtaaaaccctaggccttttacgaaagaaggaaaaga
gagctacttcctggccggacctcattgccaaggttttccggatcgatgtgaaggcagatgttgactcgatccaccccact
gagttctgccataactgctggagcatcatgcacaggaagtttagcagtgccccatgtgaggtttacttcccgaggaatgt
cactatggaatggcaccctcacacacccagctgcgacatctgcaacacagccagaagaggcctgaagcggaagt
ccctgcagcctaatctgcagctgagcaagaaactgaaaaccgtgctggaccaggccagacaggcccggcaaaga
aagagaagggcccaagccagaatcagcagcaaggacgtgatgaagaagatcgccaactgcagcaagatccac
ctgagcaccaaactgctggccgtggacttccctgagcacttcgtgaagtccatcagctgccagatctgcgagcacatc
ctggccgatcctgtggaaacaaactgcaagcacgtgttctgcagagtgtgcatcctgcggtgcctgaaagtgatgggc
agctactgcccctcctgcagatacccttgcttccccaccgatctggaaagccctgtgaagtccttcctgagcgtgctgaa
cagcctgatggtcaagtgccccgccaaagaatgcaacgaggaagtgtccctggaaaagtacaaccaccacatcag
cagccacaaagagtccaaagaaatcttcgtgcacatcaacaaaggcggcagaccccggcagcatctgctgtctctt
acaagacgggcccagaagcaccggctgagagaactgaagctgcaagtgaaggcctttgccgacaaagaggaag
gcggcgacgtcaagagcgtgtgcatgaccctgtttctgctggccctgagagcccggaatgagcatagacaggccgat
gagctggaagccatcatgcaaggcaaaggcagcggactgcagcctgctgtgtgtctggctatcagagtgaacacctt
cctgtcctgcagccagtaccacaagatgtaccggaccgtgaaggccattaccggcagacagatcttccagcctctgc
acgccctgagaaacgccgagaaagttctgctgcctggctaccaccacttcgagtggcagcctccactgaagaacgt
gtccagcagcaccgacgtgggcatcatcgatggactgagcggactgtctagcagcgtggacgactaccccgtggac
acaatcgccaagcggttcagatacgacagcgccctggtgtctgccctgatggacatggaagaggacatcctggaag
gcatgcggagccaggacctggacgattacctgaacggccctttcaccgtggtggtcaaagaaagctgtgacggcat
gggcgacgtgtccgagaaacacggatctggacctgtggtgccagagaaggccgtgcggttcagcttcaccatcatg
aagatcactatcgcccacagcagccagaacgtgaaagtgttcgaggaagccaagcctaacagcgagctgtgctgc
aagcctctgtgtctgatgctggccgacgagagcgatcacgagacactgaccgccattctgagccctctgatcgccga
acgggaagccatgaagtcctccgagctgatgctcgaactcggcggcatcctgagaaccttcaagttcatcttccgcgg
caccggctacgacgagaagctcgttagagaggtggaaggcctggaagcctctggcagcgtgtacatctgcaccctgt
gtgacgccaccagactggaagctagccagaacctggtgttccacagcatcaccagaagccacgccgaaaacctg
gaaagatacgaagtgtggcggagcaacccctaccacgagagcgtggaagaactgcgggatagagtgaagggcg
tgtccgccaagcctttcatcgagacagtgcctagcatcgacgccctgcactgcgatattggcaacgccgccgaattcta
caagatctttcagctggaaatcggcgaggtgtacaagaaccccaacgcctctaaagaggaacggaagcgctggca
ggccacactggataagcacctgagaaagaagatgaatctgaagcccatcatgaggatgaacggcaacttcgcccg
gaagctgatgaccaaagaaaccgtggatgccgtgtgcgagctgatcccctctgaggaaagacacgaggccctgcg
ggaactgatggacctgtacctgaagatgaagcccgtgtggcggtctagctgtcctgccaaagagtgccctgagtctct
gtgccagtacagcttcaacagccagagattcgccgagctgctgtccaccaagttcaagtacagatacgagggcaag
atcaccaactacttccacaagaccctggctcacgtgcccgagatcatcgagagagatggctctattggcgcctgggcc
tctgagggcaatgagtctggcaacaagctgttccggcggttccgcaagatgaacgccagacagagcaagtgctacg
agatggaagatgtgctgaagcaccactggctgtacaccagcaagtacctgcagaaattcatgaacgcccacaacgc
cctcaagaccagcggctttaccatgaatcctcaggccagcctgggcgatcctttaggcatagaggactctctggaaag
ccaagattcaatggaattttaagtagggcaaccacttatgagttggtttttgcaattgagtttccctctgggttgcattgagg
gcttctcctagcaccctttactgctgtgtatggggcttcaccatccaagaggtggtaggttggagtaagatgctacagatg
ctctcaagtcaggaatagaaactgatgagctgattgcttgaggcttttagtgagttccgaaaagcaacaggaaaaatc
agttatctgaaagctcagtaactcagaacaggagtaactgcaggggaccagagatgagcaaagatctgtgtgtgttg
gggagctgtcatgtaaatcaaagccaaggttgtcaaagaacagccagtgaggccaggaaagaaattggtcttgtggt
tttcatttttttcccccttgattgattatattttgtattgagatatgataagtgccttctatttcatttttgaataattcttcatttttata
attttacatatcttggcttgctatataagattcaaaagagctttttaaatttttctaataatatcttacatttgtacagcatgatgacct
ttacaaagtgctctcaatgcatttacccattcgttatataaatatgttacatcaggacaactttgagaaaatcagtcctttttta
tgtttaaattatgtatctattgtaaccttcagagtttaggaggtcatctgctgtcatggatttttcaataatgaatttagaataca
cctgttagctacagttagttattaaatcttctgataatatatgtttacttagctatcagaagccaagtatgattctttatttttacttt
ttcatttcaagaaatttagagtttccaaatttagagct
Exemplary polynucleotide specific for “g7 exon2 M2/3”, “g10 exon2 M2/3” and “g13
exon2 M2/3” gRNAs for the exon 2 RAG1 gene replacement strategy with long right
HA
(SEQ ID NO: 112)
gaattcttttagagctgatgagcacaacaggagatatccagtccatggtcctgtggatggtaaaaccctaggccttttac
gaaagaaggaaaagagagctacttcctggccggacctcattgccaaggttttccggatcgatgtgaaggcagatgtt
gactcgatccaccccactgagttctgccataactgctggagcatcatgcacaggaagtttagcagtgcaccatgcgaa
gtgtacttccccagaaacgtgaccatggaatggcaccctcacacacccagctgcgacatctgcaacacagccagaa
gaggcctgaagcggaagtccctgcagcctaatctgcagctgagcaagaaactgaaaaccgtgctggaccaggcca
gacaggcccggcaaagaaagagaagggcccaagccagaatcagcagcaaggacgtgatgaagaagatcgcc
aactgcagcaagatccacctgagcaccaaactgctggccgtggacttccctgagcacttcgtgaagtccatcagctgc
cagatctgcgagcacatcctggccgatcctgtggaaacaaactgcaagcacgtgttctgcagagtgtgcatcctgcgg
tgcctgaaagtgatgggcagctactgcccctcctgcagatacccttgcttccccaccgatctggaaagccctgtgaagt
ccttcctgagcgtgctgaacagcctgatggtcaagtgccccgccaaagaatgcaacgaggaagtgtccctggaaaa
gtacaaccaccacatcagcagccacaaagagtccaaagaaatcttcgtgcacatcaacaaaggggcagacccc
ggcagcatctgctgtctcttacaagacgggcccagaagcaccggctgagagaactgaagctgcaagtgaaggcctt
tgccgacaaagaggaaggcggcgacgtcaagagcgtgtgcatgaccctgtttctgctggccctgagagcccggaat
gagcatagacaggccgatgagctggaagccatcatgcaaggcaaaggcagcggactgcagcctgctgtgtgtctg
gctatcagagtgaacaccttcctgtcctgcagccagtaccacaagatgtaccggaccgtgaaggccattaccggcag
acagatcttccagcctctgcacgccctgagaaacgccgagaaagttctgctgcctggctaccaccacttcgagtggca
gcctccactgaagaacgtgtccagcagcaccgacgtgggcatcatcgatggactgagcggactgtctagcagcgtg
gacgactaccccgtggacacaatcgccaagcggttcagatacgacagcgccctggtgtctgccctgatggacatgg
aagaggacatcctggaaggcatgcggagccaggacctggacgattacctgaacggccctttcaccgtggtggtcaa
agaaagctgtgacggcatgggcgacgtgtccgagaaacacggatctggacctgtggtgccagagaaggccgtgcg
gttcagcttcaccatcatgaagatcactatcgcccacagcagccagaacgtgaaagtgttcgaggaagccaagccta
acagcgagctgtgctgcaagcctctgtgtctgatgctggccgacgagagcgatcacgagacactgaccgccattctg
agccctctgatcgccgaacgggaagccatgaagtcctccgagctgatgctcgaactcggcggcatcctgagaacctt
caagttcatcttccgcggcaccggctacgacgagaagctcgttagagaggtggaaggcctggaagcctctggcagc
gtgtacatctgcaccctgtgtgacgccaccagactggaagctagccagaacctggtgttccacagcatcaccagaag
ccacgccgaaaacctggaaagatacgaagtgtggcggagcaacccctaccacgagagcgtggaagaactgcgg
gatagagtgaagggcgtgtccgccaagcctttcatcgagacagtgcctagcatcgacgccctgcactgcgatattggc
aacgccgccgaattctacaagatctttcagctggaaatcggcgaggtctacaagaaccccaacgcctctaaagagg
aacggaagcgctggcaggccacactggataagcacctgagaaagaagatgaatctgaagcccatcatgaggatg
aacggcaacttcgcccggaagctgatgaccaaagaaaccgtggatgccgtgtgcgagctgatcccctctgaggaaa
gacacgaggccctgcgggaactgatggacctgtacctgaagatgaagcccgtgtggcggtctagctgtcctgccaaa
gagtgccctgagtctctgtgccagtacagcttcaacagccagagattcgccgagctgctgtccaccaagttcaagtac
agatacgagggcaagatcaccaactacttccacaagaccctggctcacgtgcccgagatcatcgagagagatggct
ctattggcgcctgggcctctgagggcaatgagtctggcaacaagctgttccggcggttccgcaagatgaacgccaga
cagagcaagtgctacgagatggaagatgtgctgaagcaccactggctgtacaccagcaagtacctgcagaaattca
tgaacgcccacaacgccctcaagaccagcggctttaccatgaatcctcaggccagcctgggcgatcctttaggcata
gaggactctctggaaagccaagattcaatggaattttaagtagggcaaccacttatgagttggtttttgcaattgagtttcc
ctctgggttgcattgagggcttctcctagcaccctttactgctgtgtatggggcttcaccatccaagaggtggtaggttgga
gtaagatgctacagatgctctcaagtcaggaatagaaactgatgagctgattgcttgaggcttttagtgagttccgaaaa
gcaacaggaaaaatcagttatctgaaagctcagtaactcagaacaggagtaactgcaggggaccagagatgagc
aaagatctgtgtgtgttggggagctgtcatgtaaatcaaagccaaggttgtcaaagaacagccagtgaggccaggaa
agaaattggtcttgtggttttcatttttttcccccttgattgattatattttgtattgagatatgataagtgccttctatttcatttttgaa
taattcttcatttttataattttacatatcttggcttgctatataagattcaaaagagctttttaaatttttctaataatatcttacattt
gtacagcatgatgacctttacaaagtgctctcaatgcatttacccattcgttatataaatatgttacatcaggacaactttga
gaaaatcagtccttttttatgtttaaattatgtatctattgtaaccttcagagtttaggaggtcatctgctgtcatggatttttcaat
aatgaatttagaatacacctgttagctacagttagttattaaatcttctgataatatatgtttacttagctatcagaagccaag
tatgattctttatttttactttttcatttcaagaaatttagagtttccaaatttagagcttctgcatacagtcttaaagccacagag
gcttgtaaaaatataggttagcttgatgtctaaaaatatatttcatgtcttactgaaacattttgccagactttctccaaatga
aacctgaatcaatttttctaaatctaggtttcatagagtcctctcctctgcaatgtgttattctttctataatgatcagtttactttc
agtggattcagaattgtgtagcaggataaccttgtatttttccatccgctaagtttagatggagtccaaacgcagtacagc
agaagagttaacatttacacagtgctttttaccactgtggaatgttttcacactcatttttccttacaacaattctgaggagta
ggtgttgttattatctccatttgatgggggtttaaatgatttgctcaaagtcatttaggggtaataaatacttggcttggaaattt
aacacagtccttttgtctccaaagcccttcttctttccaccacaaattaatcactatgtttataaggtagtatcagaattttttta
ggattcacaactaatcactatagcacatgaccttgggattacatttttatggggcaggggtaagcaagtttttaaatcattt
gtgtgctctggctcttttgatagaagaaagcaacacaaaagctccaaagggccccctaaccctcttgtggctccagttat
ttggaaactatgatctgcatccttaggaatctgggatttgccagttgctggcaatgtagagcaggcatggaattttatatgc
tagtgagtcataatgatatgttagtgttaattagttttttcttcctttgattttattggccataattgctactcttcatacacagtatat
caaagagcttgataatttagttgtcaaaag
Exemplary polynucleotide specific for “g8 exon2 M2/3”, “g9 exon2 M2/3” and “g12
exon2 M2/3” gRNAs for the exon 2 RAG1 gene replacement strategy with long right
HA
(SEQ ID NO: 113)
ccagtccatggtcctgtggatggtaaaaccctaggccttttacgaaagaaggaaaagagagctacttcctggccgga
cctcattgccaaggttttccggatcgatgtgaaggcagatgttgactcgatccaccccactgagttctgccataactgctg
gagcatcatgcacaggaagtttagcagtgccccatgtgaggtttacttcccgaggaacgtgaccatggagtggcacc
cccacacaccatcctgtgacatctgcaacactgctagaagaggcctgaagcggaagtccctgcagcctaatctgcag
ctgagcaagaaactgaaaaccgtgctggaccaggccagacaggcccggcaaagaaagagaagggcccaagc
cagaatcagcagcaaggacgtgatgaagaagatcgccaactgcagcaagatccacctgagcaccaaactgctgg
ccgtggacttccctgagcacttcgtgaagtccatcagctgccagatctgcgagcacatcctggccgatcctgtggaaac
aaactgcaagcacgtgttctgcagagtgtgcatcctgcggtgcctgaaagtgatgggcagctactgcccctcctgcag
atacccttgcttccccaccgatctggaaagccctgtgaagtccttcctgagcgtgctgaacagcctgatggtcaagtgcc
ccgccaaagaatgcaacgaggaagtgtccctggaaaagtacaaccaccacatcagcagccacaaagagtccaa
agaaatcttcgtgcacatcaacaaaggcggcagaccccggcagcatctgctgtctcttacaagacgggcccagaag
caccggctgagagaactgaagctgcaagtgaaggcctttgccgacaaagaggaaggcggcgacgtcaagagcgt
gtgcatgaccctgtttctgctggccctgagagcccggaatgagcatagacaggccgatgagctggaagccatcatgc
aaggcaaaggcagcggactgcagcctgctgtgtgtctggctatcagagtgaacaccttcctgtcctgcagccagtacc
acaagatgtaccggaccgtgaaggccattaccggcagacagatcttccagcctctgcacgccctgagaaacgccg
agaaagttctgctgcctggctaccaccacttcgagtggcagcctccactgaagaacgtgtccagcagcaccgacgtg
ggcatcatcgatggactgagcggactgtctagcagcgtggacgactaccccgtggacacaatcgccaagcggttca
gatacgacagcgccctggtgtctgccctgatggacatggaagaggacatcctggaaggcatgcggagccaggacc
tggacgattacctgaacggccctttcaccgtggtggtcaaagaaagctgtgacggcatgggcgacgtgtccgagaaa
cacggatctggacctgtggtgccagagaaggccgtgcggttcagcttcaccatcatgaagatcactatcgcccacag
cagccagaacgtgaaagtgttcgaggaagccaagcctaacagcgagctgtgctgcaagcctctgtgtctgatgctgg
ccgacgagagcgatcacgagacactgaccgccattctgagccctctgatcgccgaacgggaagccatgaagtcctc
cgagctgatgctcgaactcggcggcatcctgagaaccttcaagttcatcttccgcggcaccggctacgacgagaagc
tcgttagagaggtggaaggcctggaagcctctggcagcgtgtacatctgcaccctgtgtgacgccaccagactggaa
gctagccagaacctggtgttccacagcatcaccagaagccacgccgaaaacctggaaagatacgaagtgtggcgg
agcaacccctaccacgagagcgtggaagaactgcgggatagagtgaagggcgtgtccgccaagcctttcatcgag
acagtgcctagcatcgacgccctgcactgcgatattggcaacgccgccgaattctacaagatctttcagctggaaatc
ggcgaggtctacaagaaccccaacgcctctaaagaggaacggaagcgctggcaggccacactggataagcacct
gagaaagaagatgaatctgaagcccatcatgaggatgaacggcaacttcgcccggaagctgatgaccaaagaaa
ccgtggatgccgtgtgcgagctgatcccctctgaggaaagacacgaggccctgcgggaactgatggacctgtacctg
aagatgaagcccgtgtggcggtctagctgtcctgccaaagagtgccctgagtctctgtgccagtacagcttcaacagc
cagagattcgccgagctgctgtccaccaagttcaagtacagatacgagggcaagatcaccaactacttccacaaga
ccctggctcacgtgcccgagatcatcgagagagatggctctattggcgcctgggcctctgagggcaatgagtctggca
acaagctgttccggcggttccgcaagatgaacgccagacagagcaagtgctacgagatggaagatgtgctgaagc
accactggctgtacaccagcaagtacctgcagaaattcatgaacgcccacaacgccctcaagaccagcggctttac
catgaatcctcaggccagcctgggcgatcctttaggcatagaggactctctggaaagccaagattcaatggaattttaa
gtagggcaaccacttatgagttggtttttgcaattgagtttccctctgggttgcattgagggcttctcctagcaccctttactg
ctgtgtatggggcttcaccatccaagaggtggtaggttggagtaagatgctacagatgctctcaagtcaggaatagaa
actgatgagctgattgcttgaggcttttagtgagttccgaaaagcaacaggaaaaatcagttatctgaaagctcagtaa
ctcagaacaggagtaactgcaggggaccagagatgagcaaagatctgtgtgtgttggggagctgtcatgtaaatcaa
agccaaggttgtcaaagaacagccagtgaggccaggaaagaaattggtcttgtggttttcatttttttcccccttgattgat
tatattttgtattgagatatgataagtgccttctatttcatttttgaataattcttcatttttataattttacatatcttggcttgctatat
aagattcaaaagagctttttaaatttttctaataatatcttacatttgtacagcatgatgacctttacaaagtgctctcaatgc
atttacccattcgttatataaatatgttacatcaggacaactttgagaaaatcagtccttttttatgtttaaattatgtatctattgt
aaccttcagagtttaggaggtcatctgctgtcatggatttttcaataatgaatttagaatacacctgttagctacagttagtta
ttaaatcttctgataatatatgtttacttagctatcagaagccaagtatgattctttatttttactttttcatttcaagaaatttagag
tttccaaatttagagcttctgcatacagtcttaaagccacagaggcttgtaaaaatataggttagcttgatgtctaaaaata
tatttcatgtcttactgaaacattttgccagactttctccaaatgaaacctgaatcaatttttctaaatctaggtttcatagagtc
ctctcctctgcaatgtgttattctttctataatgatcagtttactttcagtggattcagaattgtgtagcaggataaccttgtatttt
tccatccgctaagtttagatggagtccaaacgcagtacagcagaagagttaacatttacacagtgctttttaccactgtg
gaatgttttcacactcatttttccttacaacaattctgaggagtaggtgttgttattatctccatttgatgggggtttaaatgattt
gctcaaagtcatttaggggtaataaatacttggcttggaaatttaacacagtccttttgtctccaaagcccttcttctttccac
cacaaattaatcactatgtttataaggtagtatcagaatttttttaggattcacaactaatcactatagcacatgaccttggg
attacatttttatggggcaggggtaagcaagtttttaaatcatttgtgtgctctggctcttttgatagaagaaagcaacacaa
aagctccaaagggccccctaaccctcttgtggctccagttatttggaaactatgatctgcatccttaggaatctgggatttg
ccagttgctggcaatgtagagcaggcatggaattttatatgctagtgagtcataatgatatgttagtgttaattagttttttctt
cctttgattttattggccataattgctactcttcatacacagtatatcaaagagcttgataatttagttgtcaaaag
Exemplary polynucleotide specific for “g11 exon2 M2/3” gRNA for the exon 2 RAG1
gene replacement strategy with long right HA
(SEQ ID NO: 114)
ctgatgagcacaacaggagatatccagtccatggtcctgtggatggtaaaaccctaggccttttacgaaagaaggaa
aagagagctacttcctggccggacctcattgccaaggttttccggatcgatgtgaaggcagatgttgactcgatccacc
ccactgagttctgccataactgctggagcatcatgcacaggaagtttagcagtgccccatgtgaggtttacttccccaga
aacgtgaccatggaatggcaccctcacacacccagctgcgacatctgcaacacagccagaagaggcctgaagcg
gaagtccctgcagcctaatctgcagctgagcaagaaactgaaaaccgtgctggaccaggccagacaggcccggc
aaagaaagagaagggcccaagccagaatcagcagcaaggacgtgatgaagaagatcgccaactgcagcaag
atccacctgagcaccaaactgctggccgtggacttccctgagcacttcgtgaagtccatcagctgccagatctgcgag
cacatcctggccgatcctgtggaaacaaactgcaagcacgtgttctgcagagtgtgcatcctgcggtgcctgaaagtg
atgggcagctactgcccctcctgcagatacccttgcttccccaccgatctggaaagccctgtgaagtccttcctgagcgt
gctgaacagcctgatggtcaagtgccccgccaaagaatgcaacgaggaagtgtccctggaaaagtacaaccacca
catcagcagccacaaagagtccaaagaaatcttcgtgcacatcaacaaaggcggcagaccccggcagcatctgct
gtctcttacaagacgggcccagaagcaccggctgagagaactgaagctgcaagtgaaggcctttgccgacaaaga
ggaaggcggcgacgtcaagagcgtgtgcatgaccctgtttctgctggccctgagagcccggaatgagcatagacag
gccgatgagctggaagccatcatgcaaggcaaaggcagcggactgcagcctgctgtgtgtctggctatcagagtga
acaccttcctgtcctgcagccagtaccacaagatgtaccggaccgtgaaggccattaccggcagacagatcttccag
cctctgcacgccctgagaaacgccgagaaagttctgctgcctggctaccaccacttcgagtggcagcctccactgaa
gaacgtgtccagcagcaccgacgtgggcatcatcgatggactgagcggactgtctagcagcgtggacgactacccc
gtggacacaatcgccaagcggttcagatacgacagcgccctggtgtctgccctgatggacatggaagaggacatcct
ggaaggcatgcggagccaggacctggacgattacctgaacggccctttcaccgtggtggtcaaagaaagctgtgac
ggcatgggcgacgtgtccgagaaacacggatctggacctgtggtgccagagaaggccgtgcggttcagcttcacca
tcatgaagatcactatcgcccacagcagccagaacgtgaaagtgttcgaggaagccaagcctaacagcgagctgt
gctgcaagcctctgtgtctgatgctggccgacgagagcgatcacgagacactgaccgccattctgagccctctgatcg
ccgaacgggaagccatgaagtcctccgagctgatgctcgaactcggcggcatcctgagaaccttcaagttcatcttcc
gcggcaccggctacgacgagaagctcgttagagaggtggaaggcctggaagcctctggcagcgtgtacatctgca
ccctgtgtgacgccaccagactggaagctagccagaacctggtgttccacagcatcaccagaagccacgccgaaa
acctggaaagatacgaagtgtggcggagcaacccctaccacgagagcgtggaagaactgcgggatagagtgaa
gggcgtgtccgccaagcctttcatcgagacagtgcctagcatcgacgccctgcactgcgatattggcaacgccgccg
aattctacaagatctttcagctggaaatcggcgaggtctacaagaaccccaacgcctctaaagaggaacggaagcg
ctggcaggccacactggataagcacctgagaaagaagatgaatctgaagcccatcatgaggatgaacggcaactt
cgcccggaagctgatgaccaaagaaaccgtggatgccgtgtgcgagctgatcccctctgaggaaagacacgaggc
cctgcgggaactgatggacctgtacctgaagatgaagcccgtgtggcggtctagctgtcctgccaaagagtgccctga
gtctctgtgccagtacagcttcaacagccagagattcgccgagctgctgtccaccaagttcaagtacagatacgaggg
caagatcaccaactacttccacaagaccctggctcacgtgcccgagatcatcgagagagatggctctattggcgcct
gggcctctgagggcaatgagtctggcaacaagctgttccggcggttccgcaagatgaacgccagacagagcaagt
gctacgagatggaagatgtgctgaagcaccactggctgtacaccagcaagtacctgcagaaattcatgaacgccca
caacgccctcaagaccagcggctttaccatgaatcctcaggccagcctgggcgatcctttaggcatagaggactctct
ggaaagccaagattcaatggaattttaagtagggcaaccacttatgagttggtttttgcaattgagtttccctctgggttgc
attgagggcttctcctagcaccctttactgctgtgtatggggcttcaccatccaagaggtggtaggttggagtaagatgct
acagatgctctcaagtcaggaatagaaactgatgagctgattgcttgaggcttttagtgagttccgaaaagcaacagg
aaaaatcagttatctgaaagctcagtaactcagaacaggagtaactgcaggggaccagagatgagcaaagatctgt
gtgtgttggggagctgtcatgtaaatcaaagccaaggttgtcaaagaacagccagtgaggccaggaaagaaattggt
cttgtggttttcatttttttcccccttgattgattatattttgtattgagatatgataagtgccttctatttcatttttgaataattcttca
tttttataattttacatatcttggcttgctatataagattcaaaagagctttttaaatttttctaataatatcttacatttgtacagcatg
atgacctttacaaagtgctctcaatgcatttacccattcgttatataaatatgttacatcaggacaactttgagaaaatcagt
ccttttttatgtttaaattatgtatctattgtaaccttcagagtttaggaggtcatctgctgtcatggatttttcaataatgaatttag
aatacacctgttagctacagttagttattaaatcttctgataatatatgtttacttagctatcagaagccaagtatgattctttat
ttttactttttcatttcaagaaatttagagtttccaaatttagagcttctgcatacagtcttaaagccacagaggcttgtaaaa
atataggttagcttgatgtctaaaaatatatttcatgtcttactgaaacattttgccagactttctccaaatgaaacctgaatc
aatttttctaaatctaggtttcatagagtcctctcctctgcaatgtgttattctttctataatgatcagtttactttcagtggattcag
aattgtgtagcaggataaccttgtatttttccatccgctaagtttagatggagtccaaacgcagtacagcagaagagtta
acatttacacagtgctttttaccactgtggaatgttttcacactcatttttccttacaacaattctgaggagtaggtgttgttatta
tctccatttgatgggggtttaaatgatttgctcaaagtcatttaggggtaataaatacttggcttggaaatttaacacagtcct
tttgtctccaaagcccttcttctttccaccacaaattaatcactatgtttataaggtagtatcagaatttttttaggattcacaac
taatcactatagcacatgaccttgggattacatttttatggggcaggggtaagcaagtttttaaatcatttgtgtgctctggct
cttttgatagaagaaagcaacacaaaagctccaaagggccccctaaccctcttgtggctccagttatttggaaactatg
atctgcatccttaggaatctgggatttgccagttgctggcaatgtagagcaggcatggaattttatatgctagtgagtcata
atgatatgttagtgttaattagttttttcttcctttgattttattggccataattgctactcttcatacacagtatatcaaagagcttg
ataatttagttgtcaaaag
Exemplary polynucleotide specific for “g14 exon2 M5” gRNA for the exon 2 RAG1 gene
replacement strategy with long right HA
(SEQ ID NO: 115)
catggagtggcacccccacacaccatcctgtgacatctgcaacactgcccgtcggggactcaagaggaagagtcttc
agccaaacttgcagctcagcaaaaaactcaaaactgtgcttgaccaagcaagacaagcccgtcagcgcaagaga
agagctcaggcaaggatcagcagcaaggatgtcatgaagaagatcgccaactgcagtaagatacatcttagtacca
agctccttgcagtggacttcccagagcactttgtgaaatccatctcctgccagatctgtgaacacattctggctgaccctg
tggagaccaactgtaagcatgtcttttgccgggtctgcattctcagatgcctcaaagtcatgggcagctattgtccctcttg
ccgatatccatgcttccctactgacctggagagtccagtgaagtcctttctgagcgtcttgaattccctgatggtgaaatgt
ccagcaaaagagtgcaatgaggaggtcaccctagaaaagtacaaccaccacatcagcagccacaaagagtcca
aagaaatcttcgtgcacatcaacaaaggggcagaccccggcagcatctgctgtctcttacaagacgggcccagaa
gcaccggctgagagaactgaagctgcaagtgaaggcctttgccgacaaagaggaaggcggcgacgtcaagagc
gtgtgcatgaccctgtttctgctggccctgagagcccggaatgagcatagacaggccgatgagctggaagccatcat
gcaaggcaaaggcagcggactgcagcctgctgtgtgtctggctatcagagtgaacaccttcctgtcctgcagccagta
ccacaagatgtaccggaccgtgaaggccattaccggcagacagatcttccagcctctgcacgccctgagaaacgcc
gagaaagttctgctgcctggctaccaccacttcgagtggcagcctccactgaagaacgtgtccagcagcaccgacgt
gggcatcatcgatggactgagcggactgtctagcagcgtggacgactaccccgtggacacaatcgccaagcggttc
agatacgacagcgccctggtgtctgccctgatggacatggaagaggacatcctggaaggcatgcggagccaggac
ctggacgattacctgaacggccctttcaccgtggtggtcaaagaaagctgtgacggcatgggcgacgtgtccgagaa
acacggatctggacctgtggtgccagagaaggccgtgcggttcagcttcaccatcatgaagatcactatcgcccaca
gcagccagaacgtgaaagtgttcgaggaagccaagcctaacagcgagctgtgctgcaagcctctgtgtctgatgctg
gccgacgagagcgatcacgagacactgaccgccattctgagccctctgatcgccgaacgggaagccatgaagtcc
tccgagctgatgctcgaactcggcggcatcctgagaaccttcaagttcatcttccgcggcaccggctacgacgagaa
gctcgttagagaggtggaaggcctggaagcctctggcagcgtgtacatctgcaccctgtgtgacgccaccagactgg
aagctagccagaacctggtgttccacagcatcaccagaagccacgccgaaaacctggaaagatacgaagtgtggc
ggagcaacccctaccacgagagcgtggaagaactgcgggatagagtgaagggcgtgtccgccaagcctttcatcg
agacagtgcctagcatcgacgccctgcactgcgatattggcaacgccgccgaattctacaagatctttcagctggaaa
tcggcgaggtctacaagaaccccaacgcctctaaagaggaacggaagcgctggcaggccacactggataagcac
ctgagaaagaagatgaatctgaagcccatcatgaggatgaacggcaacttcgcccggaagctgatgaccaaagaa
accgtggatgccgtgtgcgagctgatcccctctgaggaaagacacgaggccctgcgggaactgatggacctgtacct
gaagatgaagcccgtgtggcggtctagctgtcctgccaaagagtgccctgagtctctgtgccagtacagcttcaacag
ccagagattcgccgagctgctgtccaccaagttcaagtacagatacgagggcaagatcaccaactacttccacaag
accctggctcacgtgcccgagatcatcgagagagatggctctattggcgcctgggcctctgagggcaatgagtctggc
aacaagctgttccggcggttccgcaagatgaacgccagacagagcaagtgctacgagatggaagatgtgctgaag
caccactggctgtacaccagcaagtacctgcagaaattcatgaacgcccacaacgccctcaagaccagcggcttta
ccatgaatcctcaggccagcctgggcgatcctttaggcatagaggactctctggaaagccaagattcaatggaatttta
agtagggcaaccacttatgagttggtttttgcaattgagtttccctctgggttgcattgagggcttctcctagcaccctttact
gctgtgtatggggcttcaccatccaagaggtggtaggttggagtaagatgctacagatgctctcaagtcaggaataga
aactgatgagctgattgcttgaggcttttagtgagttccgaaaagcaacaggaaaaatcagttatctgaaagctcagta
actcagaacaggagtaactgcaggggaccagagatgagcaaagatctgtgtgtgttggggagctgtcatgtaaatca
aagccaaggttgtcaaagaacagccagtgaggccaggaaagaaattggtcttgtggttttcatttttttcccccttgattg
attatattttgtattgagatatgataagtgccttctatttcatttttgaataattcttcatttttataattttacatatcttggcttgctat
ataagattcaaaagagctttttaaatttttctaataatatcttacatttgtacagcatgatgacctttacaaagtgctctcaatg
catttacccattcgttatataaatatgttacatcaggacaactttgagaaaatcagtccttttttatgtttaaattatgtatctatt
gtaaccttcagagtttaggaggtcatctgctgtcatggatttttcaataatgaatttagaatacacctgttagctacagttagt
tattaaatcttctgataatatatgtttacttagctatcagaagccaagtatgattctttatttttactttttcatttcaagaaatttag
agtttccaaatttagagcttctgcatacagtcttaaagccacagaggcttgtaaaaatataggttagcttgatgtctaaaa
atatatttcatgtcttactgaaacattttgccagactttctccaaatgaaacctgaatcaatttttctaaatctaggtttcataga
gtcctctcctctgcaatgtgttattctttctataatgatcagtttactttcagtggattcagaattgtgtagcaggataaccttgta
tttttccatccgctaagtttagatggagtccaaacgcagtacagcagaagagttaacatttacacagtgctttttaccactg
tggaatgttttcacactcatttttccttacaacaattctgaggagtaggtgttgttattatctccatttgatgggggtttaaatgat
ttgctcaaagtcatttaggggtaataaatacttggcttggaaatttaacacagtccttttgtctccaaagcccttcttctttcca
ccacaaattaatcactatgtttataaggtagtatcagaatttttttaggattcacaactaatcactatagcacatgaccttgg
gattacatttttatggggcaggggtaagcaagtttttaaatcatttgtgtgctctggctcttttgatagaagaaagcaacaca
aaagctccaaagggccccctaaccctcttgtggctccagttatttggaaactatgatctgcatccttaggaatctgggattt
gccagttgctggcaatgtagagcaggcatggaattttatatgctagtgagtcataatgatatgttagtgttaattagttttttct
tcctttgattttattggccataattgctactcttcatacacagtatatcaaagagcttgataatttagttgtcaaaag
Exemplary polynucleotide specific for g9 gRNA for the intron 1 RAG1 gene
replacement strategy with long right HA
(SEQ ID NO: 116)
tgagcacacagttattacttggaaattgtgtacagactaagttgaagatgttaggagggaagattgtgggccaagtaac
ggggtgtatgtgtgtgggtatagggtgggcagctgggatggaaatggggggctgctgctgctgctgcaccctggcctc
ctgaactaatgatatcactcaccagaaactactgttcctgcactgtccaagccaccccaaactagtttgtcaaaatgaat
ctgtgctgtgtggagggaggcacgcctgtagctctgatgtcagatggcaatgtgaattcctgacctcttctcttcctcccac
aggccgccaccatggccgccagctttcctcctacactgggactgtctagcgcccctgacgagattcagcaccctcaca
tcaagttcagcgagtggaagttcaagctgttcagagtgcggagcttcgagaaaacccctgaggaagcccagaaaga
gaagaaggacagcttcgagggcaagcccagcctggaacagtctcctgctgtgctggataaggccgacggccaga
aacctgtgcctacacagcctctgctgaaggctcaccccaagttctccaagaagttccacgacaacgagaaggccag
aggcaaggccatccaccaggccaatctgagacacctgtgccggatctgcggcaacagcttcagagccgacgagc
acaatcggagataccctgtgcacggccctgtggatggaaagactctgggcctgctgcggaagaaagaaaagagag
ccaccagctggcccgacctgatcgccaaggtgttcagaatcgacgtgaaggccgatgtggacagcattcaccccac
cgagttctgccacaactgctggtccatcatgcaccggaagttcagctctgccccttgcgaggtgtacttccccagaaac
gtgaccatggaatggcacccacacacacccagctgcgacatctgcaacacagccagaagaggcctgaagcgga
agtccctgcagcctaatctgcagctgagcaagaaactgaaaaccgtgctggaccaggccagacaggcccggcaa
agaaaaagacgcgcccaggctagaatcagcagcaaggacgtgatgaagaagatcgccaactgcagcaagatcc
acctgagcaccaaactgctggccgtggacttccctgagcacttcgtgaagtccatcagctgccagatctgcgagcaca
tcctggccgatcctgtggaaacaaactgcaagcacgtgttctgcagagtgtgcatcctgcggtgcctgaaagtgatgg
gcagctactgccccagctgtagatacccttgcttccccaccgacctggaaagccctgtgaagtcctttctgagcgtgctg
aacagcctgatggtcaagtgccccgccaaagaatgcaacgaggaagtgtccctggaaaagtacaaccaccacatc
agcagccacaaagagtccaaagaaatcttcgtgcacatcaacaaaggcggcagaccccggcagcatctgctgtct
cttacaagaagggcccagaagcaccggctgcgggaactgaagctgcaagtgaaggcctttgccgacaaagagga
aggcggcgacgtcaagagcgtgtgcatgaccctgtttctgctggccctgagagcccggaatgagcatagacaggcc
gatgagctggaagccattatgcaaggcaaaggcagcggactgcagcctgctgtgtgtctggctatcagagtgaatac
cttcctgagctgcagccagtaccacaagatgtaccggaccgtgaaagccatcaccggcagacagatcttccagcca
ctgcacgccctgagaaacgccgagaaagttctgctgcctggctaccaccacttcgagtggcagcctccactgaagaa
cgtgtccagcagcaccgacgtgggcatcatcgatggactgtctggactgagcagcagcgtggacgattaccccgtgg
acacaatcgccaagagattcagatacgacagcgccctggtgtctgccctgatggacatggaagaggacatcctgga
aggcatgcggagccaggacctggacgactatctgaacggccctttcaccgtggtggtcaaagaaagctgtgacggc
atgggcgacgtgtccgagaaacacggatctggacctgtggtgccagagaaggccgtgcggttcagcttcaccatcat
gaagatcactatcgcccacagcagccagaacgtgaaggtgttcgaggaagccaagcctaacagcgagctgtgctg
caagcctctgtgtctgatgctggccgacgagagcgatcacgagacactgaccgccattctgagccctctgatcgccga
acgggaagccatgaagtcctccgagctgatgctcgaactcggcggcatcctgagaaccttcaagttcatcttccgcgg
caccggctacgacgagaagctcgttagagaggtggaaggcctggaagcctctggcagcgtgtacatctgcaccctgt
gtgacgccaccagactggaagctagccagaacctggtgttccacagcatcaccagaagccacgccgaaaacctcg
agagatacgaagtgtggcggagcaacccctaccacgagagcgtggaagaactgcgggatagagtgaagggcgt
gtccgccaagcctttcatcgagacagtgcctagcatcgacgccctgcactgcgatattggcaacgccgccgaattcta
caagatctttcagctggaaatcggcgaggtctacaagaaccccaacgcctctaaagaggaacggaagcgctggca
ggccacactggataagcacctgagaaagaagatgaatctgaagcccatcatgcggatgaacggcaacttcgcccg
gaagctgatgaccaaagaaaccgtggatgccgtgtgcgagctgatcccctctgaggaaagacacgaggccctgag
ggaactgatggacctgtacctgaagatgaagcccgtgtggcggtctagctgtcctgccaaagagtgccctgagtctct
gtgccagtacagcttcaacagccagagattcgccgagctgctgagcacaaagttcaagtacagatacgaggggaa
gatcacgaactacttccacaagaccctggctcacgtgcccgagatcatcgagagagatggctctattggcgcctggg
cctctgagggcaatgagtctggcaacaagctgtttcggcggttcagaaagatgaacgccagacagagcaagtgcta
cgagatggaagatgtgctgaagcaccactggctgtacaccagcaagtacctgcagaaattcatgaacgcccacaac
gccctcaagaccagcggctttaccatgaatcctcaggccagcctgggagatcctctgggcattgaggatagcctgga
atcccaggacagcatggaattctgaaggcatagaggactctctggaaagccaagattcaatggaattttaagtaggg
caaccacttatgagttggtttttgcaattgagtttccctctgggttgcattgagggcttctcctagcaccctttactgctgtgtat
ggggcttcaccatccaagaggtggtaggttggagtaagatgctacagatgctctcaagtcaggaatagaaactgatg
agctgattgcttgaggcttttagtgagttccgaaaagcaacaggaaaaatcagttatctgaaagctcagtaactcagaa
caggagtaactgcaggggaccagagatgagcaaagatctgtgtgtgttggggagctgtcatgtaaatcaaagccaa
ggttgtcaaagaacagccagtgaggccaggaaagaaattggtcttgtggttttcatttttttcccccttgattgattatattttg
tattgagatatgataagtgccttctatttcatttttgaataattcttcatttttataattttacatatcttggcttgctatataagattca
aaagagctttttaaatttttctaataatatcttacatttgtacagcatgatgacctttacaaagtgctctcaatgcatttacccat
tcgttatataaatatgttacatcaggacaactttgagaaaatcagtccttttttatgtttaaattatgtatctattgtaaccttcag
agtttaggaggtcatctgctgtcatggatttttcaataatgaatttagaatacacctgttagctacagttagttattaaatcttct
gataatatatgtttacttagctatcagaagccaagtatgattctttatttttactttttcatttcaagaaatttagagtttccaaattt
agagcttctgcatacagtcttaaagccacagaggcttgtaaaaatataggttagcttgatgtctaaaaatatatttcatgtc
ttactgaaacattttgccagactttctccaaatgaaacctgaatcaatttttctaaatctaggtttcatagagtcctctcctctg
caatgtgttattctttctataatgatcagtttactttcagtggattcagaattgtgtagcaggataaccttgtatttttccatccgc
taagtttagatggagtccaaacgcagtacagcagaagagttaacatttacacagtgctttttaccactgtggaatgttttc
acactcatttttccttacaacaattctgaggagtaggtgttgttattatctccatttgatgggggtttaaatgatttgctcaaagt
catttaggggtaataaatacttggcttggaaatttaacacagtccttttgtctccaaagcccttcttctttccaccacaaatta
atcactatgtttataaggtagtatcagaatttttttaggattcacaactaatcactatagcacatgaccttgggattacattttt
atggggcaggggtaagcaagtttttaaatcatttgtgtgctctggctcttttgatagaagaaagcaacacaaaagctcca
aagggccccctaaccctcttgtggctccagttatttggaaactatgatctgcatccttaggaatctgggatttgccagttgct
ggcaatgtagagcaggcatggaattttatatgctagtgagtcataatgatatgttagtgttaattagttttttcttcctttgatttt
attggccataattgctactcttcatacacagtatatcaaagagcttgataatttagtt
Exemplary polynucleotide specific for “g11 exon2 M2/M3” gRNA for the exon 2 RAG1
gene targeting strategy
(SEQ ID NO: 160)
ttcagcacccacatattaaattttcagaatggaaatttaagctgttccgggtgagatcctttgaaaag acacctgaagaa
gctcaaaaggaaaagaaggattcctttgaggggaaaccctctctggagcaatctccagcagtcctggacaaggctg
atggtcagaagccagtcccaactcagccattgttaaaagcccaccctaagttttcaaagaaatttcacgacaacgaga
aagcaagaggcaaagcgatccatcaagccaaccttcgacatctctgccgcatctgtgggaattcttttagagctgatg
agcacaacaggagatatccagtccatggtcctgtggatggtaaaaccctaggccttttacgaaagaaggaaaagag
agctacttcctggccggacctcattgccaaggttttccggatcgatgtgaaggcagatgttgactcgatccaccccactg
agttctgccataactgctggagcatcatgcacaggaagtttagcagtgccccatgtgaggtttacttccccagaaacgt
gaccatggaatggcaccctcacacacccagctgcgacatctgcaacacagccagaagaggcctgaagcggaagt
ccctgcagcctaatctgcagctgagcaagaaactgaaaaccgtgctggaccaggccagacaggcccggcaaaga
aagagaagggcccaagccagaatcagcagcaaggacgtgatgaagaagatcgccaactgcagcaagatccac
ctgagcaccaaactgctggccgtggacttccctgagcacttcgtgaagtccatcagctgccagatctgcgagcacatc
ctggccgatcctgtggaaacaaactgcaagcacgtgttctgcagagtgtgcatcctgcggtgcctgaaagtgatgggc
agctactgcccctcctgcagatacccttgcttccccaccgatctggaaagccctgtgaagtccttcctgagcgtgctgaa
cagcctgatggtcaagtgccccgccaaagaatgcaacgaggaagtgtccctggaaaagtacaaccaccacatcag
cagccacaaagagtccaaagaaatcttcgtgcacatcaacaaaggcggcagaccccggcagcatctgctgtctctt
acaagacgggcccagaagcaccggctgagagaactgaagctgcaagtgaaggcctttgccgacaaagaggaag
gcggcgacgtcaagagcgtgtgcatgaccctgtttctgctggccctgagagcccggaatgagcatagacaggccgat
gagctggaagccatcatgcaaggcaaaggcagcggactgcagcctgctgtgtgtctggctatcagagtgaacacctt
cctgtcctgcagccagtaccacaagatgtaccggaccgtgaaggccattaccggcagacagatcttccagcctctgc
acgccctgagaaacgccgagaaagttctgctgcctggctaccaccacttcgagtggcagcctccactgaagaacgt
gtccagcagcaccgacgtgggcatcatcgatggactgagcggactgtctagcagcgtggacgactaccccgtggac
acaatcgccaagcggttcagatacgacagcgccctggtgtctgccctgatggacatggaagaggacatcctggaag
gcatgcggagccaggacctggacgattacctgaacggccctttcaccgtggtggtcaaagaaagctgtgacggcat
gggcgacgtgtccgagaaacacggatctggacctgtggtgccagagaaggccgtgcggttcagcttcaccatcatg
aagatcactatcgcccacagcagccagaacgtgaaagtgttcgaggaagccaagcctaacagcgagctgtgctgc
aagcctctgtgtctgatgctggccgacgagagcgatcacgagacactgaccgccattctgagccctctgatcgccga
acgggaagccatgaagtcctccgagctgatgctcgaactcggcggcatcctgagaaccttcaagttcatcttccgcgg
caccggctacgacgagaagctcgttagagaggtggaaggcctggaagcctctggcagcgtgtacatctgcaccctgt
gtgacgccaccagactggaagctagccagaacctggtgttccacagcatcaccagaagccacgccgaaaacctg
gaaagatacgaagtgtggcggagcaacccctaccacgagagcgtggaagaactgcgggatagagtgaagggcg
tgtccgccaagcctttcatcgagacagtgcctagcatcgacgccctgcactgcgatattggcaacgccgccgaattcta
caagatctttcagctggaaatcggcgaggtctacaagaaccccaacgcctctaaagaggaacggaagcgctggca
ggccacactggataagcacctgagaaagaagatgaatctgaagcccatcatgaggatgaacggcaacttcgcccg
gaagctgatgaccaaagaaaccgtggatgccgtgtgcgagctgatcccctctgaggaaagacacgaggccctgcg
ggaactgatggacctgtacctgaagatgaagcccgtgtggcggtctagctgtcctgccaaagagtgccctgagtctct
gtgccagtacagcttcaacagccagagattcgccgagctgctgtccaccaagttcaagtacagatacgagggcaag
atcaccaactacttccacaagaccctggctcacgtgcccgagatcatcgagagagatggctctattggcgcctgggcc
tctgagggcaatgagtctggcaacaagctgttccggcggttccgcaagatgaacgccagacagagcaagtgctacg
agatggaagatgtgctgaagcaccactggctgtacaccagcaagtacctgcagaaattcatgaacgcccacaacgc
cctcaagaccagcggctttaccatgaatcctcaggccagcctgggcgatcctctgggaattgaggatagcctggaatc
ccaggacagcatggaattctgataaccgaggaacgtgaccatggagtggcacccccacacaccatcctgtgacatc
tgcaacactgcccgtcggggactcaagaggaagagtcttcagccaaacttgcagctcagcaaaaaactcaaaactg
tgcttgaccaagcaagacaagcccgtcagcgcaagagaagagctcaggcaaggatcagcagcaaggatgtcatg
aagaagatcgccaactgcagtaagatacatcttagtaccaagctccttgcagtggacttcccagagcactttgtgaaat
ccatctcctgccagatctgtgaacacattctggctgaccctgtggagaccaactgtaagcatgtcttttgccgggtctgca
ttctcagatgcctcaaagtcatgggcagctattgtccctcttgccgatatccatgcttccctactgacctggagagtccagt
gaagtcctttctgagcgtcttgaattccctgatggtgaaatgtccagcaaaagagtg
Exemplary polynucleotide specific for “g11 exon2 M2/M3” gRNA for the exon 2 RAG1
gene replacement strategy
(SEQ ID NO: 161)
ttcagcacccacatattaaattttcagaatggaaatttaagctgttccgggtgagatcctttgaaaagacacctgaagaa
gctcaaaaggaaaagaaggattcctttgaggggaaaccctctctggagcaatctccagcagtcctggacaaggctg
atggtcagaagccagtcccaactcagccattgttaaaagcccaccctaagttttcaaagaaatttcacgacaacgaga
aagcaagaggcaaagcgatccatcaagccaaccttcgacatctctgccgcatctgtgggaattcttttagagctgatg
agcacaacaggagatatccagtccatggtcctgtggatggtaaaaccctaggccttttacgaaagaaggaaaagag
agctacttcctggccggacctcattgccaaggttttccggatcgatgtgaaggcagatgttgactcgatccaccccactg
agttctgccataactgctggagcatcatgcacaggaagtttagcagtgccccatgtgaggtttacttccccagaaacgt
gaccatggaatggcaccctcacacacccagctgcgacatctgcaacacagccagaagaggcctgaagcggaagt
ccctgcagcctaatctgcagctgagcaagaaactgaaaaccgtgctggaccaggccagacaggcccggcaaaga
aagagaagggcccaagccagaatcagcagcaaggacgtgatgaagaagatcgccaactgcagcaagatccac
ctgagcaccaaactgctggccgtggacttccctgagcacttcgtgaagtccatcagctgccagatctgcgagcacatc
ctggccgatcctgtggaaacaaactgcaagcacgtgttctgcagagtgtgcatcctgcggtgcctgaaagtgatgggc
agctactgcccctcctgcagatacccttgcttccccaccgatctggaaagccctgtgaagtccttcctgagcgtgctgaa
cagcctgatggtcaagtgccccgccaaagaatgcaacgaggaagtgtccctggaaaagtacaaccaccacatcag
cagccacaaagagtccaaagaaatcttcgtgcacatcaacaaaggcggcagaccccggcagcatctgctgtctctt
acaagacgggcccagaagcaccggctgagagaactgaagctgcaagtgaaggcctttgccgacaaagaggaag
gcggcgacgtcaagagcgtgtgcatgaccctgtttctgctggccctgagagcccggaatgagcatagacaggccgat
gagctggaagccatcatgcaaggcaaaggcagcggactgcagcctgctgtgtgtctggctatcagagtgaacacctt
cctgtcctgcagccagtaccacaagatgtaccggaccgtgaaggccattaccggcagacagatcttccagcctctgc
acgccctgagaaacgccgagaaagttctgctgcctggctaccaccacttcgagtggcagcctccactgaagaacgt
gtccagcagcaccgacgtgggcatcatcgatggactgagcggactgtctagcagcgtggacgactaccccgtggac
acaatcgccaagcggttcagatacgacagcgccctggtgtctgccctgatggacatggaagaggacatcctggaag
gcatgcggagccaggacctggacgattacctgaacggccctttcaccgtggtggtcaaagaaagctgtgacggcat
gggcgacgtgtccgagaaacacggatctggacctgtggtgccagagaaggccgtgcggttcagcttcaccatcatg
aagatcactatcgcccacagcagccagaacgtgaaagtgttcgaggaagccaagcctaacagcgagctgtgctgc
aagcctctgtgtctgatgctggccgacgagagcgatcacgagacactgaccgccattctgagccctctgatcgccga
acgggaagccatgaagtcctccgagctgatgctcgaactcggcggcatcctgagaaccttcaagttcatcttccgcgg
caccggctacgacgagaagctcgttagagaggtggaaggcctggaagcctctggcagcgtgtacatctgcaccctgt
gtgacgccaccagactggaagctagccagaacctggtgttccacagcatcaccagaagccacgccgaaaacctg
gaaagatacgaagtgtggcggagcaacccctaccacgagagcgtggaagaactgcgggatagagtgaagggcg
tgtccgccaagcctttcatcgagacagtgcctagcatcgacgccctgcactgcgatattggcaacgccgccgaattcta
caagatctttcagctggaaatcggcgaggtctacaagaaccccaacgcctctaaagaggaacggaagcgctggca
ggccacactggataagcacctgagaaagaagatgaatctgaagcccatcatgaggatgaacggcaacttcgcccg
gaagctgatgaccaaagaaaccgtggatgccgtgtgcgagctgatcccctctgaggaaagacacgaggccctgcg
ggaactgatggacctgtacctgaagatgaagcccgtgtggcggtctagctgtcctgccaaagagtgccctgagtctct
gtgccagtacagcttcaacagccagagattcgccgagctgctgtccaccaagttcaagtacagatacgaggg caag
atcaccaactacttccacaagaccctggctcacgtgcccgagatcatcgagagagatggctctattggcgcctgggcc
tctgagggcaatgagtctggcaacaagctgttccggcggttccgcaagatgaacgccagacagagcaagtgctacg
agatggaagatgtgctgaagcaccactggctgtacaccagcaagtacctgcagaaattcatgaacgcccacaacgc
cctcaagaccagcggctttaccatgaatcctcaggccagcctgggcgatcctttaggcatagaggactctctggaaag
ccaagattcaatggaattttaagtagggcaaccacttatgagttggtttttgcaattgagtttccctctgggttgcattgagg
gcttctcctagcaccctttactgctgtgtatggggcttcaccatccaagaggtggtaggttggagtaagatgctacagatg
ctctcaagtcaggaatagaaactgatgagctgattgcttgaggcttttagtgagttccgaaaagcaacaggaaaaatc
agttatctgaaagctcagtaactcagaacaggagtaactgcaggggaccagagatgagcaaagatctgtgtgtgttg
gggagctgtcatgtaaatcaaagccaaggttgtcaaagaacagccagtgaggccaggaaagaaattggtcttgtggt
tttcatttttttcccccttgattgattatattttgtattgagatatgataagtgccttctatttcatttttgaataattcttcatttttata
attttacatatcttggcttgctatataagattcaaaagagctttttaaatttttctaataatatcttacatttgtacagcatgatgacct
ttacaaagtgctctcaatgcatttacccattcgttatataaatatgttacatcaggacaactttgagaaaatcagtcctttttta
tgtttaaattatgtatctattgtaaccttcagagtttaggaggtcatctgctgtcatggatttttcaataatgaatttagaataca
cctgttagctacagttagttattaaatcttctgataatatatgtttacttagctatcagaagccaagtatgattctttatttttacttt
ttcatttcaagaaatttagagtttccaaatttagagcttctgcatacagtcttaaagccacagaggcttgtaaaaatatagg
ttagcttgatgtctaaaaatatatttcatgtcttactgaaacattttgccagactttctccaaatgaaacctgaatcaatttttct
aaatctaggtttcatagagtcctctcctctgcaatgtgttattctttctataatgatcagtttactttcagtggattcagaattgtg
tagcaggataaccttgtatttttccatccgctaagtttagatggagtccaaacgcagtacagcagaagagtt
Exemplary polynucleotide specific for “g13 exon2 M2/M3” gRNA for the exon 2 RAG1
gene targeting strategy
(SEQ ID NO: 162)
ttcagcacccacatattaaattttcagaatggaaatttaagctgttccgggtgagatcctttgaaaagacacctgaagaa
gctcaaaaggaaaagaaggattcctttgaggggaaaccctctctggagcaatctccagcagtcctggacaaggctg
atggtcagaagccagtcccaactcagccattgttaaaagcccaccctaagttttcaaagaaatttcacgacaacgaga
aagcaagaggcaaagcgatccatcaagccaaccttcgacatctctgccgcatctgtgggaattcttttagagctgatg
agcacaacaggagatatccagtccatggtcctgtggatggtaaaaccctaggccttttacgaaagaaggaaaagag
agctacttcctggccggacctcattgccaaggttttccggatcgatgtgaaggcagatgttgactcgatccaccccactg
agttctgccataactgctggagcatcatgcacaggaagtttagcagtgcaccatgcgaagtgtacttccccagaaacg
tgaccatggaatggcaccctcacacacccagctgcgacatctgcaacacagccagaagaggcctgaagcggaag
tccctgcagcctaatctgcagctgagcaagaaactgaaaaccgtgctggaccaggccagacaggcccggcaaag
aaagagaagggcccaagccagaatcagcagcaaggacgtgatgaagaagatcgccaactgcagcaagatcca
cctgagcaccaaactgctggccgtggacttccctgagcacttcgtgaagtccatcagctgccagatctgcgagcacat
cctggccgatcctgtggaaacaaactgcaagcacgtgttctgcagagtgtgcatcctgcggtgcctgaaagtgatggg
cagctactgcccctcctgcagatacccttgcttccccaccgatctggaaagccctgtgaagtccttcctgagcgtgctga
acagcctgatggtcaagtgccccgccaaagaatgcaacgaggaagtgtccctggaaaagtacaaccaccacatca
gcagccacaaagagtccaaagaaatcttcgtgcacatcaacaaaggcggcagaccccggcagcatctgctgtctct
tacaagacgggcccagaagcaccggctgagagaactgaagctgcaagtgaaggcctttgccgacaaagaggaa
ggcggcgacgtcaagagcgtgtgcatgaccctgtttctgctggccctgagagcccggaatgagcatagacaggccg
atgagctggaagccatcatgcaaggcaaaggcagcggactgcagcctgctgtgtgtctggctatcagagtgaacac
cttcctgtcctgcagccagtaccacaagatgtaccggaccgtgaaggccattaccggcagacagatcttccagcctct
gcacgccctgagaaacgccgagaaagttctgctgcctggctaccaccacttcgagtggcagcctccactgaagaac
gtgtccagcagcaccgacgtgggcatcatcgatggactgagcggactgtctagcagcgtggacgactaccccgtgg
acacaatcgccaagcggttcagatacgacagcgccctggtgtctgccctgatggacatggaagaggacatcctgga
aggcatgcggagccaggacctggacgattacctgaacggccctttcaccgtggtggtcaaagaaagctgtgacggc
atgggcgacgtgtccgagaaacacggatctggacctgtggtgccagagaaggccgtgcggttcagcttcaccatcat
gaagatcactatcgcccacagcagccagaacgtgaaagtgttcgaggaagccaagcctaacagcgagctgtgctg
caagcctctgtgtctgatgctggccgacgagagcgatcacgagacactgaccgccattctgagccctctgatcgccga
acgggaagccatgaagtcctccgagctgatgctcgaactcggcggcatcctgagaaccttcaagttcatcttccgcgg
caccggctacgacgagaagctcgttagagaggtggaaggcctggaagcctctggcagcgtgtacatctgcaccctgt
gtgacgccaccagactggaagctagccagaacctggtgttccacagcatcaccagaagccacgccgaaaacctg
gaaagatacgaagtgtggcggagcaacccctaccacgagagcgtggaagaactgcgggatagagtgaagggcg
tgtccgccaagcctttcatcgagacagtgcctagcatcgacgccctgcactgcgatattggcaacgccgccgaattcta
caagatctttcagctggaaatcggcgaggtctacaagaaccccaacgcctctaaagaggaacggaagcgctggca
ggccacactggataagcacctgagaaagaagatgaatctgaagcccatcatgaggatgaacggcaacttcgcccg
gaagctgatgaccaaagaaaccgtggatgccgtgtgcgagctgatcccctctgaggaaagacacgaggccctgcg
ggaactgatggacctgtacctgaagatgaagcccgtgtggcggtctagctgtcctgccaaagagtgccctgagtctct
gtgccagtacagcttcaacagccagagattcgccgagctgctgtccaccaagttcaagtacagatacgagggcaag
atcaccaactacttccacaagaccctggctcacgtgcccgagatcatcgagagagatggctctattggcgcctgggcc
tctgagggcaatgagtctggcaacaagctgttccggcggttccgcaagatgaacgccagacagagcaagtgctacg
agatggaagatgtgctgaagcaccactggctgtacaccagcaagtacctgcagaaattcatgaacgcccacaacgc
cctcaagaccagcggctttaccatgaatcctcaggccagcctgggcgatcctctgggaattgaggatagcctggaatc
ccaggacagcatggaattctgataagtgaggtttacttcccgaggaacgtgaccatggagtggcacccccacacacc
atcctgtgacatctgcaacactgcccgtcggggactcaagaggaagagtcttcagccaaacttgcagctcagcaaaa
aactcaaaactgtgcttgaccaagcaagacaagcccgtcagcgcaagagaagagctcaggcaaggatcagcag
caaggatgtcatgaagaagatcgccaactgcagtaagatacatcttagtaccaagctccttgcagtggacttcccaga
gcactttgtgaaatccatctcctgccagatctgtgaacacattctggctgaccctgtggagaccaactgtaagcatgtcttt
tgccgggtctgcattctcagatgcctcaaagtcatgggcagctattgtccctcttgccgatatccatgcttccctactgacct
ggagagtccagtgaagtcctttctgagcgtcttgaattccctgatggtgaaatgt
Exemplary polynucleotide specific for “g13 exon2 M2/M3”, “g7 exon2 M2/3”, “g10
exon2 M2/3” gRNAs for the exon 2 RAG1 gene replacement strategy
(SEQ ID NO: 163)
ttcagcacccacatattaaattttcagaatggaaatttaagctgttccgggtgagatcctttgaaaagacacctgaagaa
gctcaaaaggaaaagaaggattcctttgaggggaaaccctctctggagcaatctccagcagtcctggacaaggctg
atggtcagaagccagtcccaactcagccattgttaaaagcccaccctaagttttcaaagaaatttcacgacaacgaga
aagcaagaggcaaagcgatccatcaagccaaccttcgacatctctgccgcatctgtgggaattcttttagagctgatg
agcacaacaggagatatccagtccatggtcctgtggatggtaaaaccctaggccttttacgaaagaaggaaaagag
agctacttcctggccggacctcattgccaaggttttccggatcgatgtgaaggcagatgttgactcgatccaccccactg
agttctgccataactgctggagcatcatgcacaggaagtttagcagtgcaccatgcgaagtgtacttccccagaaacg
tgaccatggaatggcaccctcacacacccagctgcgacatctgcaacacagccagaagaggcctgaagcggaag
tccctgcagcctaatctgcagctgagcaagaaactgaaaaccgtgctggaccaggccagacaggcccggcaaag
aaagagaagggcccaagccagaatcagcagcaaggacgtgatgaagaagatcgccaactgcagcaagatcca
cctgagcaccaaactgctggccgtggacttccctgagcacttcgtgaagtccatcagctgccagatctgcgagcacat
cctggccgatcctgtggaaacaaactgcaagcacgtgttctgcagagtgtgcatcctgcggtgcctgaaagtgatggg
cagctactgcccctcctgcagatacccttgcttccccaccgatctggaaagccctgtgaagtccttcctgagcgtgctga
acagcctgatggtcaagtgccccgccaaagaatgcaacgaggaagtgtccctggaaaagtacaaccaccacatca
gcagccacaaagagtccaaagaaatcttcgtgcacatcaacaaaggcggcagaccccggcagcatctgctgtctct
tacaagacgggcccagaagcaccggctgagagaactgaagctgcaagtgaaggcctttgccgacaaagaggaa
ggcggcgacgtcaagagcgtgtgcatgaccctgtttctgctggccctgagagcccggaatgagcatagacaggccg
atgagctggaagccatcatgcaaggcaaaggcagcggactgcagcctgctgtgtgtctggctatcagagtgaacac
cttcctgtcctgcagccagtaccacaagatgtaccggaccgtgaaggccattaccggcagacagatcttccagcctct
gcacgccctgagaaacgccgagaaagttctgctgcctggctaccaccacttcgagtggcagcctccactgaagaac
gtgtccagcagcaccgacgtgggcatcatcgatggactgagcggactgtctagcagcgtggacgactaccccgtgg
acacaatcgccaagcggttcagatacgacagcgccctggtgtctgccctgatggacatggaagaggacatcctgga
aggcatgcggagccaggacctggacgattacctgaacggccctttcaccgtggtggtcaaagaaagctgtgacggc
atgggcgacgtgtccgagaaacacggatctggacctgtggtgccagagaaggccgtgcggttcagcttcaccatcat
gaagatcactatcgcccacagcagccagaacgtgaaagtgttcgaggaagccaagcctaacagcgagctgtgctg
caagcctctgtgtctgatgctggccgacgagagcgatcacgagacactgaccgccattctgagccctctgatcgccga
acgggaagccatgaagtcctccgagctgatgctcgaactcggcggcatcctgagaaccttcaagttcatcttccgcgg
caccggctacgacgagaagctcgttagagaggtggaaggcctggaagcctctggcagcgtgtacatctgcaccctgt
gtgacgccaccagactggaagctagccagaacctggtgttccacagcatcaccagaagccacgccgaaaacctg
gaaagatacgaagtgtggcggagcaacccctaccacgagagcgtggaagaactgcgggatagagtgaagggcg
tgtccgccaagcctttcatcgagacagtgcctagcatcgacgccctgcactgcgatattggcaacgccgccgaattcta
caagatctttcagctggaaatcggcgaggtctacaagaaccccaacgcctctaaagaggaacggaagcgctggca
ggccacactggataagcacctgagaaagaagatgaatctgaagcccatcatgaggatgaacggcaacttcgcccg
gaagctgatgaccaaagaaaccgtggatgccgtgtgcgagctgatcccctctgaggaaagacacgaggccctgcg
ggaactgatggacctgtacctgaagatgaagcccgtgtggcggtctagctgtcctgccaaagagtgccctgagtctct
gtgccagtacagcttcaacagccagagattcgccgagctgctgtccaccaagttcaagtacagatacgagggcaag
atcaccaactacttccacaagaccctggctcacgtgcccgagatcatcgagagagatggctctattggcgcctgggcc
tctgagggcaatgagtctggcaacaagctgttccggcggttccgcaagatgaacgccagacagagcaagtgctacg
agatggaagatgtgctgaagcaccactggctgtacaccagcaagtacctgcagaaattcatgaacgcccacaacgc
cctcaagaccagcggctttaccatgaatcctcaggccagcctgggcgatcctttaggcatagaggactctctggaaag
ccaagattcaatggaattttaagtagggcaaccacttatgagttggtttttgcaattgagtttccctctgggttgcattgagg
gcttctcctagcaccctttactgctgtgtatggggcttcaccatccaagaggtggtaggttggagtaagatgctacagatg
ctctcaagtcaggaatagaaactgatgagctgattgcttgaggcttttagtgagttccgaaaagcaacaggaaaaatc
agttatctgaaagctcagtaactcagaacaggagtaactgcaggggaccagagatgagcaaagatctgtgtgtgttg
gggagctgtcatgtaaatcaaagccaaggttgtcaaagaacagccagtgaggccaggaaagaaattggtcttgtggt
tttcatttttttcccccttgattgattatattttgtattgagatatgataagtgccttctatttcatttttgaataattcttcatttttata
attttacatatcttggcttgctatataagattcaaaagagctttttaaatttttctaataatatcttacatttgtacagcatgatgacct
ttacaaagtgctctcaatgcatttacccattcgttatataaatatgttacatcaggacaactttgagaaaatcagtcctttttta
tgtttaaattatgtatctattgtaaccttcagagtttaggaggtcatctgctgtcatggatttttcaataatgaatttagaataca
cctgttagctacagttagttattaaatcttctgataatatatgtttacttagctatcagaagccaagtatgattctttatttttacttt
ttcatttcaagaaatttagagtttccaaatttagagcttctgcatacagtcttaaagccacagaggcttgtaaaaatatagg
ttagcttgatgtctaaaaatatatttcatgtcttactgaaacattttgccagactttctccaaatgaaacctgaatcaatttttct
aaatctaggtttcatagagtcctctcctctgcaatgtgttattctttctataatgatcagtttactttcagtggattcagaattgtg
tagcaggataaccttgtatttttccatccgctaagtttagatggagtccaaacgcagtacagcagaagagtt

### Variants, Derivatives, Analogues, and Fragments

In addition to the specific proteins and nucleotides mentioned herein, the invention also encompasses variants, derivatives, and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one of its endogenous functions. For example, a variant of RAG1 may retain the ability to form a RAG complex, mediate DNA-binding to the RSS, and introduce a double-strand break between the RSS and the adjacent coding segment. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions. For example, a derivative of RAG1 may retain the ability to form a RAG complex, mediate DNA-binding to the RSS, and introduce a double-strand break between the RSS and the adjacent coding segment.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and in the same line in the third column may be substituted for each other:

| | | |
|---|---|---|
| ALIPHATIC | Non-polar | GAP |
| | | ILV |
| | Polar-uncharged | CSTM |
| | | NQ |
| | Polar-charged | DE |
| | | KRH |
| AROMATIC | | FWY |

Typically, a variant may have a certain identity with the wild type amino acid sequence or the wild type nucleotide sequence.

In the present context, a variant sequence is taken to include an amino acid sequence which may be at least 50%, 55%, 65%, 75%, 85% or 90% identical, suitably at least 95%, 96% or 97% or 98% or 99% identical to the subject sequence. Although a variant can also be considered in terms of similarity (i.e. amino acid residues having similar chemical properties/functions), in the context of the present invention it is preferred to express in terms of sequence identity.

In the present context, a variant sequence is taken to include a nucleotide sequence which may be at least 50%, 55%, 65%, 75%, 85% or 90% identical, suitably at least 95%, 96% or 97% or 98% or 99% identical to the subject sequence. Although a variant can also be considered in terms of similarity, in the context of the present invention it is preferred to express it in terms of sequence identity.

Suitably, reference to a sequence which has a percent identity to any one of the SEQ ID NOs detailed herein refers to a sequence which has the stated percent identity over the entire length of the SEQ ID NO referred to.

Sequence identity comparisons can be conducted by eye, or more usually, with the aid of readily available sequence comparison programs. These commercially available computer programs can calculate percent identity between two or more sequences.

Percent identity may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent identity when a global alignment is performed. Consequently, most sequence comparison methods are designed to produce optimal alignments that take into consideration possible insertions and deletions without penalising unduly the overall identity score. This is achieved by inserting “gaps” in the sequence alignment to try to maximise local identity.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent identity therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410), EMBOSS Needle (Madeira, F., et al., 2019. Nucleic acids research, 47(W1), pp.W636-W641) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent identity can be measured, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent sequence identity. The software typically does this as part of the sequence comparison and generates a numerical result. The percent sequence identity may be calculated as the number of identical residues as a percentage of the total residues in the SEQ ID NO referred to.

“Fragments” are also variants and the term typically refers to a selected region of the polypeptide or polynucleotide that is of interest. “Fragment” thus refers to an amino acid or nucleic acid sequence that is a portion of a full-length polypeptide or polynucleotide.

Such variants, derivatives, and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Vector

In one aspect, the present invention provides a vector comprising the polynucleotide of the invention.

The vector may be suitable for editing a genome using the polynucleotide of the invention. The vector may be used to deliver the polynucleotide into the cell. Subsequently, the nucleotide sequence insert can be introduced into a genome at a site of a double strand break (DSB) by homology-directed repair (HDR).

The vector of the present invention may be capable of transducing mammalian cells, for example human cells. Suitably, the vector of the present invention is capable of transducing HSCs, HPCs, and/or LPCs. Suitably, the vector of the present invention is capable of transducing CD34+ cells. Suitably, the vector of the present invention is capable of transducing NALM6, K562, and/or other human cell lines (e.g. Molt4, U937, etc.). Suitably, the vector of the present invention is capable of transducing T cells.

Suitably, the vector of the present invention is a viral vector. The vector of the invention may be an adeno-associated viral (AAV) vector, although it is contemplated that other viral vectors may be used e.g. lentiviral vectors (e.g. IDLV vectors), or single or double stranded DNA.

The vector of the present invention may be in the form of a viral vector particle. Suitably, the viral vector of the present invention is in the form of an AAV vector particle. Suitably, the viral vector of the present invention is in the form of a lentiviral vector particle, for example an IDLV vector particle.

Methods of preparing and modifying viral vectors and viral vector particles, such as those derived from AAV, are well known in the art. Suitable methods are described in Ayuso, E., et al., 2010. Current gene therapy, 10(6), pp. 423-436, Merten, O. W., et al., 2016. Molecular Therapy-Methods & Clinical Development, 3, p.16017; and Nadeau, I. and Kamen, A., 2003. Biotechnology advances, 20(7-8), pp. 475-489.

### Adeno-Associated Viral (AAV) Vectors

The vector of the present invention may be an adeno-associated viral (AAV) vector. Optionally, the vector is an AAV6 vector. The vector of the present invention may be in the form of an AAV vector particle. Optionally, the vector is in the form of an AAV6 vector particle.

The AAV vector or AAV vector particle may comprise an AAV genome or a fragment or derivative thereof. An AAV genome is a polynucleotide sequence, which may encode functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome of the AAV vector of the invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

AAVs occurring in nature may be classified according to various biological systems. The AAV genome may be from any naturally derived serotype, isolate or clade of AAV.

AAV may be referred to in terms of their serotype. A serotype corresponds to a variant subspecies of AAV which, owing to its profile of expression of capsid surface antigens, has a distinctive reactivity which can be used to distinguish it from other variant subspecies. Typically, an AAV vector particle having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10 and AAV11. The AAV vector of the invention may be an AAV6 serotype.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. ITRs may be the only sequences required in cis next to the therapeutic gene. Suitably, one or more ITR sequences flank the polynucleotide of the invention.

The AAV genome may also comprise packaging genes, such as rep and/or cap genes which encode packaging functions for an AAV particle. A promoter may be operably linked to each of the packaging genes. Specific examples of such promoters include the p5, p19 and p40 promoters. For example, the p5 and p19 promoters are generally used to express the rep gene, while the p40 promoter is generally used to express the cap gene. The rep gene encodes one or more of the proteins Rep78, Rep68, Rep52 and Rep40 or variants thereof. The cap gene encodes one or more capsid proteins such as VP1, VP2 and VP3 or variants thereof.

The AAV genome may be the full genome of a naturally occurring AAV. For example, a vector comprising a full AAV genome may be used to prepare an AAV vector or vector particle.

Suitably, the AAV genome is derivatised for the purpose of administration to patients. Such derivatisation is standard in the art and the invention encompasses the use of any known derivative of an AAV genome, and derivatives which could be generated by applying techniques known in the art. The AAV genome may be a derivative of any naturally occurring AAV. Suitably, the AAV genome is a derivative of AAV1, AAV2, AAV3, AAV4, AAV5, AAV6, AAV7, AAV8, AAV9, AAV10, or AAV11. Suitably, the AAV genome is a derivative of AAV6.

Derivatives of an AAV genome include any truncated or modified forms of an AAV genome which allow for expression of a transgene from an AAV vector of the invention in vivo. Typically, it is possible to truncate the AAV genome significantly to include minimal viral sequence yet retain the above function. This may reduce the risk of recombination of the vector with wild-type virus, and avoid triggering a cellular immune response by the presence of viral gene proteins in the target cell.

Typically, a derivative will include at least one inverted terminal repeat sequence (ITR), optionally more than one ITR, such as two ITRs or more. One or more of the ITRs may be derived from AAV genomes having different serotypes, or may be a chimeric or mutant ITR. A suitable mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The AAV genome may comprise one or more ITR sequences from any naturally derived serotype, isolate or clade of AAV or a variant thereof. The AAV genome may comprise at least one, such as two, AAV1, AAV2, AAV3, AAV4, AAV5, AAV6, AAV7, AAV8, AAV9, AAV10, or AAV11 ITRs, or variants thereof.

The one or more ITRs may flank the nucleotide sequence of the invention at either end. The inclusion of one or more ITRs is can aid concatamer formation of the AAV vector in the nucleus of a host cell, for example following the conversion of single-stranded vector DNA into double-stranded DNA by the action of host cell DNA polymerases. The formation of such episomal concatamers protects the AAV vector during the life of the host cell, thereby allowing for prolonged expression of the transgene in vivo.

Suitably, ITR elements will be the only sequences retained from the native AAV genome in the derivative. Suitably, a derivative may not include the rep and/or cap genes of the native genome and any other sequences of the native genome. This may reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: one inverted terminal repeat (ITR) sequence, the replication (rep) and capsid (cap) genes. However, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the AAV vector may be tolerated in a therapeutic setting.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The AAV vector particle may be encapsidated by capsid proteins. Suitably, the AAV vector particles may be transcapsidated forms wherein an AAV genome or derivative having an ITR of one serotype is packaged in the capsid of a different serotype. The AAV vector particle also includes mosaic forms wherein a mixture of unmodified capsid proteins from two or more different serotypes makes up the viral capsid. The AAV vector particle also includes chemically modified forms bearing ligands adsorbed to the capsid surface. For example, such ligands may include antibodies for targeting a particular cell surface receptor.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector). The AAV vector may be in the form of a pseudotyped AAV vector particle.

Chimeric, shuffled or capsid-modified derivatives will be typically selected to provide one or more desired functionalities for the AAV vector. Thus, these derivatives may display increased efficiency of gene delivery and/or decreased immunogenicity (humoral or cellular) compared to an AAV vector comprising a naturally occurring AAV genome. Increased efficiency of gene delivery, for example, may be effected by improved receptor or co-receptor binding at the cell surface, improved internalisation, improved trafficking within the cell and into the nucleus, improved uncoating of the viral particle and improved conversion of a single-stranded genome to double-stranded form.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence. The unrelated protein or peptide may advantageously be one which acts as a ligand for a particular cell type, thereby conferring improved binding to a target cell or improving the specificity of targeting of the vector to a particular cell population. The unrelated protein may also be one which assists purification of the viral particle as part of the production process, i.e. an epitope or affinity tag. The site of insertion will typically be selected so as not to interfere with other functions of the viral particle e.g. internalisation, trafficking of the viral particle.

The capsid protein may be an artificial or mutant capsid protein. The term “artificial capsid” as used herein means that the capsid particle comprises an amino acid sequence which does not occur in nature or which comprises an amino acid sequence which has been engineered (e.g. modified) from a naturally occurring capsid amino acid sequence. In other words the artificial capsid protein comprises a mutation or a variation in the amino acid sequence compared to the sequence of the parent capsid from which it is derived where the artificial capsid amino acid sequence and the parent capsid amino acid sequences are aligned. The AAV vector particle may comprise an AAV6 capsid protein.

### Retroviral and Lentiviral Vectors

The vector of the present invention may be a retroviral vector or a lentiviral vector. The vector of the present invention may be a retroviral vector particle or a lentiviral vector particle.

A retroviral vector may be derived from or may be derivable from any suitable retrovirus. A large number of different retroviruses have been identified. Examples include murine leukaemia virus (MLV), human T-cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV).

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective.

Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells. In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Suitably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

As examples of lentivirus-based vectors, HIV-1- and HIV-2-based vectors are described below.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

Optionally, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Optionally, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Optionally, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

The vectors may be integration-defective. Integration defective lentiviral vectors (IDLVs) can be produced, for example, either by packaging the vector with catalytically inactive integrase (such as an HIV integrase bearing the D64V mutation in the catalytic site) or by modifying or deleting essential att sequences from the vector LTR, or by a combination of the above.

### Adenoviral Vectors

The vector of the present invention may be an adenoviral vector. The vector of the present invention may be an adenoviral vector particle.

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10⁵². Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

### Herpes Simplex Viral Vector

The vector of the present invention may be a herpes simplex viral vector. The vector of the present invention may be a herpes simplex viral vector particle.

Herpes simplex virus (HSV) is a neurotropic DNA virus with favorable properties as a gene delivery vector. HSV is highly infectious, so HSV vectors are efficient vehicles for the delivery of exogenous genetic material to cells. Viral replication is readily disrupted by null mutations in immediate early genes that in vitro can be complemented in trans, enabling straightforward production of high-titre pure preparations of non-pathogenic vector. The genome is large (152 Kb) and many of the viral genes are dispensable for replication in vitro, allowing their replacement with large or multiple transgenes. Latent infection with wild-type virus results in episomal viral persistence in sensory neuronal nuclei for the duration of the host lifetime. The vectors are non-pathogenic, unable to reactivate and persist long-term. The latency active promoter complex can be exploited in vector design to achieve long-term stable transgene expression in the nervous system. HSV vectors transduce a broad range of tissues because of the wide expression pattern of the cellular receptors recognized by the virus. Increasing understanding of the processes involved in cellular entry has allowed targeting the tropism of HSV vectors.

### Vaccinia Virus Vectors

The vector of the present invention may be a vaccinia viral vector. The vector of the present invention may be a vaccinia viral vector particle.

Vaccinia virus is large enveloped virus that has an approximately 190 kb linear, double-stranded DNA genome. Vaccinia virus can accommodate up to approximately 25 kb of foreign DNA, which also makes it useful for the delivery of large genes.

A number of attenuated vaccinia virus strains are known in the art that are suitable for gene therapy applications, for example the MVA and NYVAC strains.

### RNA-Guided Gene Editing

The vector of the present invention may be used to deliver a polynucleotide into a cell. Subsequently, a nucleotide sequence insert can be introduced into the cell's genome at a site of a double strand break (DSB) by homology-directed repair (HDR). The site of the double-strand break (DSB) can be introduced specifically by any suitable technique, for example by using an RNA-guided gene editing system.

An “RNA-guided gene editing system” can be used to introduce a DSB and typically comprises a guide RNA and a RNA-guided nuclease. A CRISPR/Cas9 system is an example of a commonly used RNA-guided gene editing system, but other RNA-guided gene editing systems may also be used.

### Guide RNAs

A “guide RNA” (gRNA) confers target sequence specificity to a RNA-guided nuclease. Guide RNAs are non-coding short RNA sequences which bind to the complementary target DNA sequences. For example, in the CRISPR/Cas9 system, guide RNA first binds to the Cas9 enzyme and the gRNA sequence guides the resulting complex via base-pairing to a specific location on the DNA, where Cas9 performs its nuclease activity by cutting the target DNA strand.

The term “guide RNA” encompasses any suitable gRNA that can be used with any RNA-guided nuclease, and not only those gRNAs that are compatible with a particular nuclease such as Cas9.

The guide RNA may comprise a trans-activating CRISPR RNA (tracrRNA) that provides the stem loop structure and a target-specific CRISPR RNA (crRNA) designed to cleave the gene target site of interest. The tracrRNA and crRNA may be annealed, for example by heating them at 95° C. for 5 minutes and letting them slowly cool down to room temperature for 10 minutes. Alternatively, the guide RNA may be a single guide RNA (sgRNA) that consists of both the crRNA and tracrRNA as a single construct.

The guide RNA may comprise of a 3′-end, which forms a scaffold for nuclease binding, and a 5′-end which is programmable to target different DNA sites. For example, the targeting specificity of CRISPR-Cas9 may be determined by the 15-25 bp sequence at the 5′ end of the guide RNA. The desired target sequence typically precedes a protospacer adjacent motif (PAM) which is a short DNA sequence usually 2-6 bp in length that follows the DNA region targeted for cleavage by the CRISPR system, such as CRISPR-Cas9. The PAM is required for a Cas nuclease to cut and is typically found 3-4 bp downstream from the cut site. After base pairing of the guide RNA to the target, Cas9 mediates a double strand break about 3-nt upstream of PAM.

Numerous tools exist for designing guide RNAs (e.g. Cui, Y., et al., 2018. Interdisciplinary Sciences: Computational Life Sciences, 10(2), pp. 455-465). For example, COSMID is a web-based tool for identifying and validating guide RNAs (Cradick T J, et al. Mol Ther—Nucleic Acids. 2014; 3(12):e214).

A list of exemplary guide RNAs for use in the present invention is provided below in Tables 13-15.

TABLE 13
Exemplary guide RNAs for exon 2 strategies

| | | +/− | |
|---|---|---|---|
| Guide | Sequence | strand | DSB site |
| g1 M5 ex2 | TTGCTGGACATTTCACCATCAGG | − | chr11: 36,574,368-36,574,369 |
| | (SEQ ID NO: 117) | | |
| g2 M5 ex2 | TGCTGGACATTTCACCATCAGGG | − | chr11: 36,574,367-36,574,368 |
| | (SEQ ID NO: 118) | | |
| g3 M5 ex2 | TCCAGCAAAAGAGTGCAATGAGG | + | chr11: 36,574,394-36,574,395 |
| | (SEQ ID NO: 119) | | |
| g4 M4 ex2 | AAGCATGGATATCGGCAAGAGGG | − | chr11: 36,574,294-36,574,295 |
| | (SEQ ID NO: 120) | | |
| g5 M3 ex2 | AAGATGTATCTTACTGCAGTTGG | − | chr11: 36,574,109-36,574,110 |
| | (SEQ ID NO: 121) | | |
| g6 M2 ex2 | CGAGGAACGTGACCATGGAGTGG | + | chr11: 36,573,910-36,573,911 |
| | (SEQ ID NO: 122) | | |
| g7 exon2 | GTTTAGCAGTGCCCCATGTGAGG | + | chr11: 36,573,878-36,573,879 |
| M2/3 | (SEQ ID NO: 123) | | |
| g8 exon2 | CTTCCTCTTGAGTCCCCGACGGG | − | chr11: 36,573,959-36,573,960 |
| M2/3 | (SEQ ID NO: 124) | | |
| g9 exon2 | ATCTGCAACACTGCCCGTCGGGG | + | chr11: 36,573,957-36,573,958 |
| M2/3 | (SEQ ID NO: 125) | | |
| g10 exon2 | TCGGGAAGTAAACCTCACATGGG | − | chr11: 36,573,879-36,573,880 |
| M2/3 | (SEQ ID NO: 126) | | |
| g11 exon2 | CATGTGAGGTTTACTTCCCGAGG | + | chr11: 36,573,892-36,573,893 |
| M2/3 | (SEQ ID NO: 127) | | |
| g12 exon2 | ACATCTGCAACACTGCCCGTCGG | + | chr11: 36,573,955-36,573,956 |
| M2/3 | (SEQ ID NO: 128) | | |
| g13 exon2 | CGGGAAGTAAACCTCACATGGGG | − | chr11: 36,573,878-36,573,879 |
| M2/3 | (SEQ ID NO: 129) | | |
| g14 exon2 | GTGCAATGAGGAGGTCAGTTTGG | + | chr11: 36,574,406-36,574,407 |
| M5 | (SEQ ID NO: 130) | | |

TABLE 14
Exemplary guide RNAs for intron 1 strategies

| | | +/− | |
|---|---|---|---|
| Guide | Sequence | strand | DSB site |
| 9 | TCAGATGGCAATGTCGAGA (SEQ ID NO: 131) | + | chr 11: 36569296-36569297 |
| 1 | TTTTCCGGATCGATGTGA (SEQ ID NO: 132) | + | chr 11: 36573791-36573792 |
| 2 | GACATCTCTGCCGCATCTG (SEQ ID NO: 133) | + | chr 11: 36573642-36573643 |
| 3 | GTGGGTGCTGAATTTCATC (SEQ ID NO: 134) | − | chr 11: 36573352-36573353 |
| 4 | GATTGTGGGCCAAGTAACG (SEQ ID NO: 135) | + | chr 11: 36569081-36569082 |
| 5 | GAAAGTCACTGTTGGTCGA (SEQ ID NO: 136) | − | chr 11: 36572473-36572474 |
| 6 | CAATTTTGAGGTGTTCGTT (SEQ ID NO: 137) | + | chr 11: 36571459-36571460 |
| 7 | GGGTTGAGTTCAACCTAAG (SEQ ID NO: 138) | + | chr 11: 36571367-36571368 |
| 8 | TTAGCCTCATTGTACTAGC (SEQ ID NO: 139) | − | chr 11: 36572860-36572861 |
| 10 | GCAATTTTGAGGTGTTCGT (SEQ ID NO: 140) | + | chr 11: 36571458-36571459 |
| 11 | ACCAGCCTCGGGATCTCAA (SEQ ID NO: 141) | − | chr 11: 36569352-36569353 |
| 12 | TCAAATCAGTCGGGTTTCC (SEQ ID NO: 142) | + | chr 11: 36572376-36572377 |

TABLE 15
Exemplary optional guide RNAs for replacement strategies

| | | +/− | |
|---|---|---|---|
| Guide | Sequence | strand | DSB site |
| g1 ex2 | GAGAGTCCTCTATGCCTAATGGG (SEQ ID | − | chr11: 36,576,390- |
| | NO: 143) | | 36,576,391 |
| g2 ex2 | AGGGGACCCATTAGGCATAGAGG (SEQ | + | chr11: 36,576,395- |
| | ID NO: 144) | | 36,576,396 |
| g3 ex2 | AGAGAGTCCTCTATGCCTAATGG (SEQ ID | − | chr11: 36,576,391- |
| | NO: 145) | | 36,576,392 |
| g1 3′UTR | AAGCCCTCAATGCAACCCAGAGG (SEQ ID | − | chr11: 36,576,484- |
| | NO: 146) | | 36,576,485 |
| g2 3′UTR | AGCCCTCAATGCAACCCAGAGGG (SEQ | − | chr11: 36,576,483- |
| | ID NO: 147) | | 36,576,484 |
| g3 3′UTR | TAGGGCAACCACTTATGAGTTGG (SEQ ID | + | chr11: 36,576,454- |
| | NO: 148) | | 36,576,455 |

For example, sequences for guides 9, 3 and 7 may be extended as shown below, for example when used as crRNA:

| | | +/− | |
|---|---|---|---|
| Guide | Sequence | strand | DSB site |
| 9 | GTCAGATGGCA | + | chr 11: |
| | ATGTCGAGA | | 36569296- |
| | (SEQ ID NO: 149) | | 36569297 |
| 3 | TGTGGGTGCTG | − | chr 11: |
| | AATTTCATC | | 36573352- |
| | (SEQ ID | | 36573353 |
| | NO: 150) | | |
| 7 | GGGGTTGAGTT | + | chr 11: |
| | CAACCTAAG | | 36571367- |
| | (SEQ ID | | 36571368 |
| | NO: 151) | | |

In one aspect, the present invention provides a guide RNA comprising or consisting of a nucleotide sequence that has at least 90% identity or at least 95% identity to any of SEQ ID NOs: 117-151. In some embodiments, the guide RNA comprises or consists of the nucleotide sequence of any of SEQ ID NOs: 117-151.

In one aspect, the present invention provides a guide RNA comprising or consisting of a nucleotide sequence that has at least 90% identity or at least 95% identity to any of SEQ ID NOs: 117-130. In some embodiments, the guide RNA comprises or consists of the nucleotide sequence of any of SEQ ID NOs: 117-130.

In one aspect, the present invention provides a guide RNA comprising or consisting of a nucleotide sequence that has at least 90% identity or at least 95% identity to SEQ ID NO: 121. In some embodiments, the guide RNA comprises or consists of the nucleotide sequence of SEQ ID NO: 121.

In one aspect, the present invention provides a guide RNA comprising or consisting of a nucleotide sequence that has at least 90% identity or at least 95% identity to SEQ ID NO: 122. In some embodiments, the guide RNA comprises or consists of the nucleotide sequence of SEQ ID NO: 122.

In one aspect, the present invention provides a guide RNA comprising or consisting of a nucleotide sequence that has at least 90% identity or at least 95% identity to SEQ ID NO: 127 or 129. In some embodiments, the guide RNA comprises or consists of the nucleotide sequence of SEQ ID NO: 127 or 129.

In one aspect, the present invention provides a guide RNA comprising or consisting of a nucleotide sequence that has at least 90% identity or at least 95% identity to SEQ ID NO: 127. In some embodiments, the guide RNA comprises or consists of the nucleotide sequence of SEQ ID NO: 127.

In one aspect, the present invention provides a guide RNA comprising or consisting of a nucleotide sequence that has at least 90% identity or at least 95% identity to SEQ ID NO: 129. In some embodiments, the guide RNA comprises or consists of the nucleotide sequence of SEQ ID NO: 129.In one aspect, the present invention provides a guide RNA comprising or consisting of a nucleotide sequence that has at least 90% identity or at least 95% identity to any of SEQ ID NOs: 131-143 or 149-151. In some embodiments, the guide RNA comprises or consists of the nucleotide sequence of any of SEQ ID NOs: 131-143 or 149-151.

In one aspect, the present invention provides a guide RNA comprising or consisting of a nucleotide sequence that has at least 90% identity or at least 95% identity to any of SEQ ID NOs: 143-148. In some embodiments, the guide RNA comprises or consists of the nucleotide sequence of any of SEQ ID NOs: 143-148.

Suitably, the guide RNA is chemically modified. The chemical modification may enhance the stability of the guide RNA. For example, from one to five (e.g. three) of the terminal nucleotides at 5′ end and/or 3′ end of the guide RNA may be chemically modified to enhance stability.

Any chemical modification which enhances the stability of the guide RNA may be used. For example, the chemical modification may be modification with 2′-O-methyl 3′-phosphorothioate, as described in Hendel A, et al. Nat Biotechnol. 2015; 33(9):985-9.

### RNA-Guided Nuclease

A “nuclease” is an enzyme that can cleave the phosphodiester bond present within a polynucleotide chain. Suitably, the nuclease is an endonuclease. Endonucleases are capable of breaking the bond from the middle of a chain.

An “RNA-guided nuclease” is a nuclease which can be directed to a specific site by a guide RNA. The present invention can be implemented using any suitable RNA-guided nuclease, for example any RNA-guided nuclease described in Murugan, K., et al., 2017. Molecular cell, 68(1), pp. 15-25. RNA-guided nucleases include, but are not limited to, Type II CRISPR nucleases such as Cas9, and Type V CRISPR nucleases such as Cas12a and Cas12b, as well as other nucleases derived therefrom. RNA-guided nucleases can be defined, in broad terms, by their PAM specificity and cleavage activity.

Suitably, the RNA-guided nuclease is a Type II CRISPR nuclease, for example a Cas9 nuclease. Cas9 is a dual RNA-guided endonuclease enzyme associated with the Clustered Regularly Interspaced Short Palindromic Repeats (CRISPR) adaptive immune system. Cas9 nucleases include the well-characterized ortholog from Streptococcus pyogenes (SpCas9). SpCas9 and other orthologs (including SaCas9, FnCa9, and AnaCas9) have been reviewed by Jiang, F. and Doudna, J. A., 2017. Annual review of biophysics, 46, pp. 505-529.

The RNA-guided nuclease may be in a complex with the guide RNA, i.e. the guide RNA and the RNA-guided nuclease may together form a ribonucleoprotein (RNP). Suitably, the RNP is a Cas9 RNP. A RNP may be formed by any method known in the art, for example by incubating a RNA-guided nuclease with a guide RNA for 5-30 minutes at room temperature. Delivering Cas9 as a preassembled RNP can protect the guide RNA from intracellular degradation thus improving stability and activity of the RNA-guided nuclease (Kim S, et al. Genome Res. 2014; 24(6):1012-9).

### Kit, Composition, Gene-Editing System

In one aspect, the present invention provides a kit, composition, or gene-editing system comprising the polynucleotide of the invention, the vector of the invention, and/or the guide RNA of the invention.

As used herein, a “gene-editing system” is a system which comprises all components necessary to edit a genome using the polynucleotide of the invention.

In some embodiments, the kit, composition, or gene-editing system comprises a polynucleotide and/or vector of the invention and a guide RNA. The guide RNA may correspond to the same DSB site targeted by the homology arms. For example, in some embodiments the kit, composition, or gene-editing system comprises:
(i) a polynucleotide comprising from 5′ to 3′: a first homology region, a nucleotide sequence encoding a RAG1 polypeptide fragment, and a second homology region, wherein the first homology region is homologous to a region upstream of chr 11:36574368 and the second homology region is homologous to a region downstream of chr 11:36574369, and/or a vector comprising said polynucleotide; and a guide RNA which comprises or consists of a nucleotide sequence that has at least 90% identity, at least 95% identity or 100% identity to SEQ ID NO: 117;
(ii) a polynucleotide comprising from 5′ to 3′: a first homology region, a nucleotide sequence encoding a RAG1 polypeptide fragment, and a second homology region, wherein the first homology region is homologous to a region upstream of chr 11:36574367 and the second homology region is homologous to a region downstream of chr 11:36574368, and/or a vector comprising said polynucleotide; and a guide RNA which comprises or consists of a nucleotide sequence that has at least 90% identity, at least 95% identity or 100% identity to SEQ ID NO: 118;
(iii) a polynucleotide comprising from 5′ to 3′: a first homology region, a nucleotide sequence encoding a RAG1 polypeptide fragment, and a second homology region, wherein the first homology region is homologous to a region upstream of chr 11:36574394 and the second homology region is homologous to a region downstream of chr 11:36574395, and/or a vector comprising said polynucleotide; and a guide RNA which comprises or consists of a nucleotide sequence that has at least 90% identity, at least 95% identity or 100% identity to SEQ ID NO: 119;
(iv) a polynucleotide comprising from 5′ to 3′: a first homology region, a nucleotide sequence encoding a RAG1 polypeptide fragment, and a second homology region, wherein the first homology region is homologous to a region upstream of chr 11:36574294 and the second homology region is homologous to a region downstream of chr 11:36574295, and/or a vector comprising said polynucleotide; and a guide RNA which comprises or consists of a nucleotide sequence that has at least 90% identity, at least 95% identity or 100% identity to SEQ ID NO: 120;
(v) a polynucleotide comprising from 5′ to 3′: a first homology region, a nucleotide sequence encoding a RAG1 polypeptide fragment, and a second homology region, wherein the first homology region is homologous to a region upstream of chr 11:36574109 and the second homology region is homologous to a region downstream of chr 11:36574110, and/or a vector comprising said polynucleotide; and a guide RNA which comprises or consists of a nucleotide sequence that has at least 90% identity, at least 95% identity or 100% identity to SEQ ID NO: 121;
(vi) a polynucleotide comprising from 5′ to 3′: a first homology region, a nucleotide sequence encoding a RAG1 polypeptide fragment, and a second homology region, wherein the first homology region is homologous to a region upstream of chr 11:36573910 and the second homology region is homologous to a region downstream of chr 11:36573911, and/or a vector comprising said polynucleotide; and a guide RNA which comprises or consists of a nucleotide sequence that has at least 90% identity, at least 95% identity or 100% identity to SEQ ID NO: 122;
(vii) a polynucleotide comprising from 5′ to 3′: a first homology region, a nucleotide sequence encoding a RAG1 polypeptide fragment, and a second homology region, wherein the first homology region is homologous to a region upstream of chr 11:36573878 and the second homology region is homologous to a region downstream of chr 11:36573879, and/or a vector comprising said polynucleotide; and a guide RNA which comprises or consists of a nucleotide sequence that has at least 90% identity, at least 95% identity or 100% identity to SEQ ID NO: 123;
(viii) a polynucleotide comprising from 5′ to 3′: a first homology region, a nucleotide sequence encoding a RAG1 polypeptide fragment, and a second homology region, wherein the first homology region is homologous to a region upstream of chr 11:36573959 and the second homology region is homologous to a region downstream of chr 11:36573960, and/or a vector comprising said polynucleotide; and a guide RNA which comprises or consists of a nucleotide sequence that has at least 90% identity, at least 95% identity or 100% identity to SEQ ID NO: 124;
(ix) a polynucleotide comprising from 5′ to 3′: a first homology region, a nucleotide sequence encoding a RAG1 polypeptide fragment, and a second homology region, wherein the first homology region is homologous to a region upstream of chr 11:36573957 and the second homology region is homologous to a region downstream of chr 11:36573958, and/or a vector comprising said polynucleotide; and a guide RNA which comprises or consists of a nucleotide sequence that has at least 90% identity, at least 95% identity or 100% identity to SEQ ID NO: 125;
(x) a polynucleotide comprising from 5′ to 3′: a first homology region, a nucleotide sequence encoding a RAG1 polypeptide fragment, and a second homology region, wherein the first homology region is homologous to a region upstream of chr 11:36573879 and the second homology region is homologous to a region downstream of chr 11:36573880, and/or a vector comprising said polynucleotide; and a guide RNA which comprises or consists of a nucleotide sequence that has at least 90% identity, at least 95% identity or 100% identity to SEQ ID NO: 126;
(xi) a polynucleotide comprising from 5′ to 3′: a first homology region, a nucleotide sequence encoding a RAG1 polypeptide fragment, and a second homology region, wherein the first homology region is homologous to a region upstream of chr 11:36573892 and the second homology region is homologous to a region downstream of chr 11:36573893, and/or a vector comprising said polynucleotide; and a guide RNA which comprises or consists of a nucleotide sequence that has at least 90% identity, at least 95% identity or 100% identity to SEQ ID NO: 127;
(xii) a polynucleotide comprising from 5′ to 3′: a first homology region, a nucleotide sequence encoding a RAG1 polypeptide fragment, and a second homology region, wherein the first homology region is homologous to a region upstream of chr 11:36573955 and the second homology region is homologous to a region downstream of chr 11:36573956, and/or a vector comprising said polynucleotide; and a guide RNA which comprises or consists of a nucleotide sequence that has at least 90% identity, at least 95% identity or 100% identity to SEQ ID NO: 128;
(xiii) a polynucleotide comprising from 5′ to 3′: a first homology region, a nucleotide sequence encoding a RAG1 polypeptide fragment, and a second homology region, wherein the first homology region is homologous to a region upstream of chr 11:36573878 and the second homology region is homologous to a region downstream of chr 11:36573879, and/or a vector comprising said polynucleotide; and a guide RNA which comprises or consists of a nucleotide sequence that has at least 90% identity, at least 95% identity or 100% identity to SEQ ID NO: 129; or
(xiv) a polynucleotide comprising from 5′ to 3′: a first homology region, a nucleotide sequence encoding a RAG1 polypeptide fragment, and a second homology region, wherein the first homology region is homologous to a region upstream of chr 11:36574406 and the second homology region is homologous to a region downstream of chr 11:36574407, and/or a vector comprising said polynucleotide; and a guide RNA which comprises or consists of a nucleotide sequence that has at least 90% identity, at least 95% identity or 100% identity to SEQ ID NO: 130.

In some embodiments the kit, composition, or gene-editing system comprises:
(v) a polynucleotide comprising from 5′ to 3′: a first homology region, a nucleotide sequence encoding a RAG1 polypeptide fragment, and a second homology region, wherein the first homology region is homologous to a region upstream of chr 11:36574109 and the second homology region is homologous to a region downstream of chr 11:36574110, and/or a vector comprising said polynucleotide; and a guide RNA which comprises or consists of a nucleotide sequence that has at least 90% identity, at least 95% identity or 100% identity to SEQ ID NO: 121; or
(vi) a polynucleotide comprising from 5′ to 3′: a first homology region, a nucleotide sequence encoding a RAG1 polypeptide fragment, and a second homology region, wherein the first homology region is homologous to a region upstream of chr 11:36573910 and the second homology region is homologous to a region downstream of chr 11:36573911, and/or a vector comprising said polynucleotide; and a guide RNA which comprises or consists of a nucleotide sequence that has at least 90% identity, at least 95% identity or 100% identity to SEQ ID NO: 122.

In some embodiments the kit, composition, or gene-editing system comprises:
(xi) a polynucleotide comprising from 5′ to 3′: a first homology region, a nucleotide sequence encoding a RAG1 polypeptide fragment, and a second homology region, wherein the first homology region is homologous to a region upstream of chr 11:36573892 and the second homology region is homologous to a region downstream of chr 11:36573893, and/or a vector comprising said polynucleotide; and a guide RNA which comprises or consists of a nucleotide sequence that has at least 90% identity, at least 95% identity or 100% identity to SEQ ID NO: 127; or
(xiii) a polynucleotide comprising from 5′ to 3′: a first homology region, a nucleotide sequence encoding a RAG1 polypeptide fragment, and a second homology region, wherein the first homology region is homologous to a region upstream of chr 11:36573878 and the second homology region is homologous to a region downstream of chr 11:36573879, and/or a vector comprising said polynucleotide; and a guide RNA which comprises or consists of a nucleotide sequence that has at least 90% identity, at least 95% identity or 100% identity to SEQ ID NO: 129.

In some embodiments the kit, composition, or gene-editing system comprises a polynucleotide comprising from 5′ to 3′: a first homology region, a nucleotide sequence encoding a RAG1 polypeptide fragment, and a second homology region, wherein the first homology region is homologous to a region upstream of chr 11:36573878 and the second homology region is homologous to a region downstream of chr 11:36573879, and/or a vector comprising said polynucleotide; and a guide RNA which comprises or consists of a nucleotide sequence that has at least 90% identity, at least 95% identity or 100% identity to SEQ ID NO: 129.

In some embodiments the kit, composition, or gene-editing system comprises:
(i) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36569295 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 131 or 149 (preferably SEQ ID NO: 131);
(ii) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36573790 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436 and/or a vector comprising said polynucleotide; and a guide RNA which comprises or consists of a nucleotide sequence that has at least 90% identity, at least 95% identity or 100% identity to SEQ ID NO: 132;
(iii) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36573641 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 133;
(iv) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36573351 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 134 or 150 (preferably SEQ ID NO: 134);
(v) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36569080 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 135;
(vi) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36572472 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 136;
(vii) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36571458 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 137;
(viii) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36571366 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 138 or 151 (preferably SEQ ID NO: 138);
(ix) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36572859 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 139;
(x) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36571457 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 140;
(xi) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36569351 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 141; or
(xii) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36572375 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 142.

In some embodiments the kit, composition, or gene-editing system comprises:
(i) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36569295 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 131 or 149 (preferably SEQ ID NO: 131);
(ii) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36573351 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 134 or 150 (preferably SEQ ID NO: 134);
(iii) a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36571366 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 138 or 151 (preferably SEQ ID NO: 138).

In some embodiments the kit, composition, or gene-editing system comprises a polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a region upstream of chr 11:36569295 and the second homology region is homologous to a region downstream of chr 11:36574557, downstream of chr 11:36574870, downstream of chr 11:36575183, downstream of chr 11:36575496, downstream of chr 11:36575810, downstream of chr 11:36576123, or downstream of chr 11:36576436, and/or a vector comprising said polynucleotide; and a guide RNA which comprises or consists of a nucleotide sequence that has at least 90% identity, at least 95% identity or 100% identity to SEQ ID NO: 131 or 149 (preferably SEQ ID NO: 131).

The kit, composition, or gene-editing system may further comprise a second guide RNA, for example when the second homology region is homologous to a region distantly downstream of the DSB (e.g. a replacement strategy). For example, the kit, composition, or gene-editing system may further comprise a guide RNA which comprises or consists of a nucleotide sequence that has at least 90% identity, at least 95% identity or 100% identity to any of SEQ ID NOs: 143-148. In some embodiments, the kit, composition, or gene-editing system further comprises a guide RNA which comprises or consists of the nucleotide sequence of any of SEQ ID NOs: 143-148.

The kit, composition, or gene-editing system may further comprise an RNA-guided nuclease. Suitably, the RNA-guided nuclease corresponds to the guide RNA used. For example, if the guide RNA comprises or consists of a nucleotide sequence that has at least 90% identity, at least 95% identity or 100% identity to any one of SEQ ID NOs: 117-151, the RNA-guided nuclease is suitably a Cas9 endonuclease.

The RNA-guided nuclease may be in a complex with the guide RNA, i.e. the guide RNA and the RNA-guided nuclease together form a ribonucleoprotein (RNP).

### Cell

In one aspect, the present invention provides a cell which has been edited using the polynucleotide, vector, kit, composition, or gene-editing system of the present invention.

In a related aspect, the present invention provides a cell comprising the polynucleotide, vector and/or genome of the present invention.

Suitably, the cell is an isolated cell. Suitably, the cell is a mammalian cell, for example a human cell.

Suitably, the cell is a hematopoietic stem cell (HSC), a hematopoietic progenitor cell (HPC), or a lymphoid progenitor cell (LPC). In some embodiments, the cell is a HSC or a HPC, optionally the cell is a HSC.

As used herein “hematopoietic stem cells” are stem cells that have no differentiation potential to cells other than hematopoietic cells, “hematopoietic progenitor cells” are progenitor cells that have no differentiation potential to cells other than hematopoietic cells, and “lymphoid progenitor cells” are progenitor cells that have no differentiation potential to cells other than lymphocytes.

The cell can be obtained from any source. The cell may be autologous or allogeneic. The cell may be obtained or obtainable from any biological sample, such as peripheral blood or cord blood. Peripheral blood may be treated with mobilising agent, i.e. may be mobilised peripheral blood. The cell may be a universal cell.

The cell may be isolated or isolatable using commercially available antibodies that bind to cell surface antigens, e.g. CD34, using methods known to those of skill in the art. For example, the antibodies may be conjugated to magnetic beads and immunological procedures utilized to recover the desired cell type. Suitably, the cell is identified by the presence or absence of one or more antigenic markers. Suitable antigenic markers include CD34, CD133, CD90, CD45, CD4, CD19, CD13, CD3, CD56, CD14, CD61/41, CD135, CD45RA, CD33, CD66b, CD38, CD45, CD10, CD11c, CD19, CD7, and CD71.

Suitably, the cell is identified by the presence of the antigenic marker CD34 (CD34⁺), i.e. the cell is a CD34+ cell. For example, the cell may be a cord blood CD34+ cell or a (mobilised) peripheral blood CD34+ cell. The cell may be a CD34+ HSC, a CD34+ HPC, or a CD34+ LPC, optionally the cell is a CD34+ HSC.

In some embodiments, the cell is identified by the presence of CD34 and the presence or absence or one or more further antigenic markers. The further antigenic markers may be selected from one or more of CD133, CD90, CD3, CD56, CD14, CD61/41, CD135, CD45RA, CD33, CD66b, CD38, CD45, CD10, CD11c, CD19, CD7, and CD71. For example, the cell may be a CD34+CD133+CD90+ cell, a CD34+CD133+CD90− cell, or a CD34+CD133-CD90-cell.

Suitably, the cell is a NALM6 cell, a K562 cell, or other human cell (e.g. a Molt4 cell, a U937 cell, etc.). Suitably, the cell is a T cell.

### Population of Cells

In one aspect, the present invention provides a population or cells comprising the cell of the present invention. Suitably, at least 1%, at least 2%, at least 5%, at least 10%, or at least 20% of the cells in the population of cells are cells of the present invention. Suitably, the population of cells comprises at least 10×10⁵, at least 50×10⁵, or at least 100×10⁵ cells of the present invention.

In a related aspect, the present invention provides a population of cells which have been edited using the polynucleotide, vector, kit, composition, or gene-editing system of the present invention. Suitably, at least 1%, at least 2%, at least 5%, at least 10%, or at least 20% of the cells in the population of cells are cells which have been edited using the polynucleotide, vector, kit, composition, or gene-editing system of the present invention. Suitably, the population of cells comprises at least 10×10⁵, at least 50×10⁵, or at least 100×10⁵ cells which have been edited using the polynucleotide, vector, kit, composition, or gene-editing system of the present invention.

In a related aspect, the present invention provides a population of cells comprising the polynucleotide, vector and/or genome of the present invention. Suitably, at least 1%, at least 2%, at least 5%, at least 10%, or at least 20% of the cells in the population of cells are cells comprising the polynucleotide, vector and/or genome of the present invention. Suitably, the population of cells comprises at least 10×10⁵, at least 50×10⁵, or at least 100×10⁵ cells comprising the polynucleotide, vector and/or genome of the present invention.

Suitably, the population of cells are mammalian cells, for example human cells. The population of cells may be autologous or allogeneic. Suitably, the population of cells are obtained or obtainable from (mobilised) peripheral blood or cord blood. The population of cells may be universal cells.

Suitably, at least 50%, at least 60%, at least 70%, or at least 80% of the population of cells are HSCs, HPCs, and/or LPCs. Suitably, at least 50%, at least 60%, at least 70%, or at least 80% of the population of cells are CD34+ cells.

In some embodiments, at least 1%, at least 2%, at least 5%, at least 10%, or at least 20% of the population of cells are CD34+ cells comprising the polynucleotide, vector and/or genome of the present invention. For example, in some embodiments at least 20% of the population of cells are CD34+ cells comprising the genome of the present invention.

In some embodiments, the population of cells comprises at least 10×10⁵, at least 50×10⁵, or at least 100×10⁵ CD34+ cells comprising the polynucleotide, vector and/or genome of the present invention. For example, in some embodiments the population of cells comprises at least 100×10⁵ CD34+ cells comprising the genome of the present invention.

### Method of Gene Editing

In one aspect, the present invention provides a method of gene editing a cell or a population of cells using polynucleotides, vectors, guide RNAs, kits, compositions and/or gene-editing system of the present invention. The present invention also provide a population of gene-edited cells obtained or obtainable by said methods.

In another aspect the present invention provides use of a polynucleotide, vector, guide RNA, kit, composition, and/or gene-editing system of the present invention for gene editing a cell or a population of cells.

Suitably, the method of gene editing a cell or a population of cells comprises:
(a) providing a cell or a population of cells; and
(b) using a kit, composition, and/or gene-editing system described herein to obtain a gene-edited cell or a population of gene-edited cells.

For example, the method of gene editing a cell or a population of cells comprises:
(a) providing a cell or a population of cells; and
(b) delivering an RNA-guided nuclease, a guide RNA, and/or a polynucleotide or vector of the present invention to the cell or population of cells to obtain a gene-edited cell or a population of gene-edited cells.

The gene-edited cell or population of gene-edited cells may be as defined herein. The present invention also provides a gene-edited cell or population of gene-edited cells obtained or obtainable by said method.

### Step (a) Providing a Cell or a Population of Cells

The population of cells may be obtained or obtainable from any suitable source. Suitably, the population of cells are obtained or obtainable from (mobilised) peripheral blood or cord blood. The population of cells may be obtained or obtainable from a subject, e.g. a subject to be treated. Suitably, the population of cells may be isolated and/or enriched from a biological sample by any method known in the art, for example by FACS and/or magnetic bead sorting.

Suitably, the population of cells are mammalian cells, for example human cells. The population of cells may be, for example, autologous or allogeneic. The population of cells may be, for example, universal cells.

Suitably, the population of cells comprises about 1×10⁵ cells per well to about 10×10⁵ cells per well, e.g. about 2×10⁵ cells per well, or about 5×10⁵ cells per well.

The population of cells may comprise HSCs, HPCs, and/or LPCs. Suitably, at least 50%, at least 60%, at least 70%, or at least 80% of the population of cells are HSCs, HPCs, and/or LPCs. In some embodiments, the population of cells consists essentially of HSCs, HPCs, and/or LPCs, or consists of HSCs, HPCs, and/or LPCs.

The population of cells may comprise CD34+ cells, e.g. CD34+ HSCs, HPCs, and/or LPCs. Suitably, at least 50%, at least 60%, at least 70%, or at least 80% of the population of cells are CD34+ cells, e.g. CD34+ HSCs, HPCs, and/or LPCs. In some embodiments, the population of cells consists essentially of CD34+ cells, e.g. CD34+ HSCs, HPCs, and/or LPCs, or consists of CD34+ cells, e.g. CD34+ HSCs, HPCs, and/or LPCs.

The population of cells may comprise CD34+CD133+CD90+ cells, CD34+CD133+CD90-cells, and/or CD34+CD133-CD90−. Suitably, at least 50%, at least 60%, at least 70%, or at least 80% of the population of cells are CD34+CD133+CD90+ cells, CD34+CD133+CD90-cells, and/or CD34+CD133−CD90− cells. In some embodiments, the population of cells consists essentially of CD34+CD133+CD90+ cells, CD34+CD133+CD90− cells, and/or CD34+CD133−CD90− cells, or consists of CD34+CD133+CD90+ cells, CD34+CD133+CD90− cells, and/or CD34+CD133−CD90− cells.

The cell or population of cells may be cultured prior to step (b). The pre-culturing step may comprise a pre-activation step and/or a pre-expansion step, optionally the pre-culturing step is a pre-activation step.

As used herein, a “pre-culturing step” refers to a culturing step which occurs prior to genetic modification of the cells. As used herein, a “pre-activating step” refers to an activation step or stimulation step which occurs prior to genetic modification of the cells. As used herein, a “pre-expansion step” refers to an expansion step which occurs prior to genetic modification of the cells.

Suitably, the method may comprise:
(a1) providing a population of cells;
(a2) pre-culturing (e.g. pre-activating and/or pre-expanding) the population of cells to obtain a pre-cultured (e.g. pre-activated and/or pre-expanded) population of cells;
(b) delivering an RNA-guided nuclease, a guide RNA, and/or a polynucleotide or vector of the present invention to the pre-cultured (e.g. pre-activated and/or pre-expanded) population of cells to obtain a population of gene-edited cells.

The pre-culturing step (e.g. pre-activation step and/or pre-expansion step) may be carried out using any suitable conditions.

During the pre-culturing step (e.g. pre-activation step and/or pre-expansion step) the population of cells may be seeded at a concentration of about 1×10⁵ cells/ml to about 10×10⁵ cells/ml, e.g. about 2×10⁵ cells/ml, or about 5×10⁵ cells/ml.

Suitably, the pre-culturing step (e.g. pre-activation step and/or pre-expansion step) is at least 1 day, at least 2 days, or at least 3 days. Suitably, the population of cells are pre-cultured (e.g. pre-activated and/or pre-expanded) for about 3 days. Suitably, the population of cells are pre-cultured in a 5% CO₂ humidified atmosphere at 37° C.

Any suitable culture medium may be used. For example, commercially available medium such as StemSpan medium may be used, which contains bovine serum albumin, insulin, transferrin, and supplements in Iscove's MDM. The culture medium may be supplemented with one or more antibiotic (e.g. penicillin, streptomycin).

The pre-culturing step (e.g. pre-activation step and/or pre-expansion step) may be carried out in the presence in of one or more cytokines and/or growth factors. As used herein, a “cytokine” is any cell signalling substance and includes chemokines, interferons, interleukins, lymphokines, and tumour necrosis factors. As used herein, a “growth factor” is any substance capable of stimulating cell proliferation, wound healing, or cellular differentiation. The terms “cytokine” and “growth factor” may overlap.

The pre-culturing step (e.g. pre-activation step and/or pre-expansion step) may be carried out in the presence of one or more early-acting cytokine, one or more transduction enhancer, and/or one or more expansion enhancer.

### Early-Acting Cytokines

As used herein, an “early-acting cytokine” is a cytokine which stimulates HSCs, HPCS, and/or LPCs or CD34+ cells. Early-acting cytokines include thrombopoietin (TPO), stem cell factor (SCF), Flt3-ligand (FLT3-L), interleukin (IL)-3, and IL-6. In some embodiments, the pre-culturing step (e.g. pre-activation step and/or pre-expansion step) is carried out in the presence of at least one early-acting cytokine. Any suitable concentration of early-acting cytokine may be used. For example, 1-1000 ng/ml, or 10-1000 ng/ml, or 10-500 ng/ml.

In some embodiments, the pre-culturing step (e.g. pre-activation step and/or pre-expansion step) is carried out in the presence of SCF. The concentration of SCF may be about 10-1000 ng/ml, about 50-500 ng/ml, or about 100-300 ng/ml.

In some embodiments, the pre-culturing step (e.g. pre-activation step and/or pre-expansion step) is carried out in the presence of FLT3-L. The concentration of FLT3-L may be about 10-1000 ng/ml, about 50-500 ng/ml, or about 100-300 ng/ml.

In some embodiments, the pre-culturing step (e.g. pre-activation step and/or pre-expansion step) is carried out in the presence of TPO. The concentration of TPO may be about 5-500 ng/ml, about 10-200 ng/ml, or about 20-100 ng/ml.

In some embodiments, the pre-culturing step (e.g. pre-activation step and/or pre-expansion step) is carried out in the presence of IL-3. The concentration of IL-3 may be about 10-200 ng/ml, about 20-100 ng/ml, or about 60 ng/ml.

In some embodiments, the pre-culturing step (e.g. pre-activation step and/or pre-expansion step) is carried out in the presence of IL-6. The concentration of IL-6 may be about 5-100 ng/ml, about 10-50 ng/ml, or about 20 ng/ml.

In some embodiments, the pre-culturing step (e.g. pre-activation step and/or pre-expansion step) is carried out in the presence of SCF (e.g. in a concentration of about 100 ng/ml), FLT3-L (e.g. in a concentration of about 100 ng/ml), TPO (e.g. in a concentration of about 20 ng/ml) and IL-6 (e.g. in a concentration of about 20 ng/ml), in particular when the population of cells are cord-blood CD34+ cells.

In some embodiments, the pre-culturing step (e.g. pre-activation step and/or pre-expansion step) is carried out in the presence of SCF (e.g. in a concentration of about 300 ng/ml), FLT3-L (e.g. in a concentration of about 300 ng/ml), TPO (e.g. in a concentration of about 100 ng/ml) and IL-3 (e.g. in a concentration of about 60 ng/ml), in particular when the population of cells are (mobilised) peripheral blood CD34+ cells.

### Transduction Enhancers

As used herein, a “transduction enhancer” is a substance that is capable of improving viral transduction of HSCs, HPCS, and/or LPCs or CD34+ cells. Suitable transduction enhancers include LentiBOOST, prostaglandin E2 (PGE2), protamine sulfate (PS), Vectofusin-1, ViraDuctin, RetroNectin, staurosporine (Stauro), 7-hydroxy-stauro, human serum albumin, polyvinyl alcohol, and cyclosporin H (CsH). In some embodiments, the pre-culturing step (e.g. pre-activation step and/or pre-expansion step) is carried out in the presence of at least one transduction enhancer. Any suitable concentration of transduction enhancer may be used, for example as described in Schott, J. W., et al., 2019. Molecular Therapy-Methods & Clinical Development, 14, pp. 134-147 or Yang, H., et al., 2020. Molecular Therapy-Nucleic Acids, 20, pp. 451-458.

In some embodiments, the pre-culturing step (e.g. pre-activation step and/or pre-expansion step) is carried out in the presence of PGE2. Suitably, the PGE2 is 16,16-dimethyl prostaglandin E2 (dmPGE2). The concentration of PGE2 may be about 1-100 μM, about 5-20 μM, or about 10 μM.

In some embodiments, the pre-culturing step (e.g. pre-activation step and/or pre-expansion step) is carried out in the presence of CsH. The concentration of CsH may be about 1-50 μM, 5-50 μM, about 10-50 μM, or about 10 μM.

### Expansion Enhancers

As used herein, an “expansion enhancer” is a substance that is capable of improving expansion of HSCs, HPCS, and/or LPCs or CD34+ cells. Suitable expansion enhancers include UM171, UM729, StemRegenin1 (SR1), diethylaminobenzaldehyde (DEAB), LG1506, BIO (GSK3β inhibitor), NR-101, trichostatin A (TSA), garcinol (GAR), valproic acid (VPA), copper chelator, tetraethylenepentamine, and nicotinamide. In some embodiments, the pre-culturing step (e.g. pre-activation step and/or pre-expansion step) is carried out in the presence of at least one expansion enhancer. Any suitable concentration of expansion enhancer may be used, for example as described in Huang, X., et al., 2019. F1000Research, 8, 1833.

In some embodiments, the pre-culturing step (e.g. pre-activation step and/or pre-expansion step) is carried out in the presence of UM171 or UM729. The concentration of UM171 may be about 10-200 nM, about 20-100 nM, or about 50 nM.

In some embodiments, the pre-culturing step (e.g. pre-activation step and/or pre-expansion step) is carried out in the presence of SR1. The concentration of SR1 may be about 0.1-10 μM, about 0.5-5 μM, or about 1 μM.

In some embodiments, the pre-culturing step (e.g. pre-activation step and/or pre-expansion step) is carried out in the presence of UM171 (e.g. in a concentration of about 50 nM) or UM729 and SR1 (e.g. in a concentration of about 1 μM).

In some embodiments, the pre-culturing step (e.g. pre-activation step and/or pre-expansion step) is carried out in the presence of SCF (e.g. in a concentration of about 100 ng/ml), FLT3-L (e.g. in a concentration of about 100 ng/ml), TPO (e.g. in a concentration of about 20 ng/ml), IL-6 (e.g. in a concentration of about 20 ng/ml), PGE2 (e.g. in a concentration of about 10 μM), UM171 (e.g. in a concentration of about 50 nM), and SR1 (e.g. in a concentration of about 1 μM), in particular when the population of cells are cord-blood CD34⁺ cells.

In some embodiments, the pre-culturing step (e.g. pre-activation step and/or pre-expansion step) is carried out in the presence of SCF (e.g. in a concentration of about 300 ng/ml), FLT3-L (e.g. in a concentration of about 300 ng/ml), TPO (e.g. in a concentration of about 100 ng/ml), IL-3 (e.g. in a concentration of about 60 ng/ml), PGE2 (e.g. in a concentration of about 10 μM), UM171 (e.g. in a concentration of about 50 nM), and SR1 (e.g. in a concentration of about 1 μM), in particular when the population of cells are (mobilised) peripheral blood CD34⁺ cells.

### Step (b) Obtaining a Gene-Edited Cell or a Population of Gene-Edited Cells

A kit, composition, and/or gene-editing system comprising an RNA-guided nuclease, a guide RNA, and/or a polynucleotide or vector of the present invention may, for example, be used to obtain the gene-edited cell or a population of gene-edited cells.

The RNA-guided nuclease, guide RNA, and/or polynucleotide or vector may be any suitable combination described herein. The guide RNA may correspond to the same DSB site targeted by the homology arms. The RNA-guided nuclease may correspond to the guide RNA used.

In some embodiments, for example when a replacement strategy is being used, a second guide RNA may be used cutting just upstream the right homology arm in combination with the first gRNA. For example, the method may further comprise delivering a second guide RNA which comprises or consists of a nucleotide sequence that has at least 90% identity, at least 95% identity or 100% identity to any of SEQ ID NOs: 143-148. In some embodiments, the method further comprises delivering a guide RNA which comprises or consists of the nucleotide sequence of any of SEQ ID NOs: 143-148.

Delivery of a RNA-Guided Nuclease, Guide RNA(s), and/or Polynucleotide or Vector

The RNA-guided nuclease, guide RNA(s), and/or polynucleotide or vector may be delivered to the cell by any suitable technique. For example, the RNA-guided nuclease may be delivered directly using electroporation, microinjection, bead loading or the like, or indirectly via transfection and/or transduction. The guide RNA(s), and/or polynucleotide or vector may be introduced by transfection and/or transduction.

As used herein “transfection” is a process using a non-viral vector to deliver a polypeptide and/or polynucleotide to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) and combinations thereof.

As used herein “transduction” is a process using a viral vector to deliver a polynucleotide to a target cell. Typical transduction methods include infection with recombinant viral vectors, such as adeno-associated viral, retroviral, lentiviral, adenoviral, baculoviral and herpes simplex viral vectors.

The RNA-guided nuclease and the guide RNA(s) may be delivered by any suitable method, for instance any method described in Wilbie, D., et al., 2019. Accounts of chemical research, 52(6), pp. 1555-1564. Suitably, the RNA-guided nuclease and the guide RNA(s) are delivered together preassembled as in the form of a RNP complex. The RNP complex may be delivered by electroporation.

Any suitable dose of the RNA-guided nuclease and/or the guide RNA(s) may be used. For example, the guide RNA(s) may be delivered at a dose of about 10-100 pmol/well, optionally about 50 pmol/well. For example, the RNP may be delivered at a dose of about 1-10 μM, optionally 1-2.5 μM.

The RNA-guided nuclease and/or the guide RNA(s) may be delivered prior to the vector and/or simultaneously with the polynucleotide or vector of the invention. Suitably, the RNA-guided nuclease and/or the guide RNA(s) are delivered prior to the polynucleotide or vector. For example, the RNA-guided nuclease and/or the guide RNA(s) may be delivered about 1-100 minutes, about 5-30, or about 15 minutes, prior to the polynucleotide or vector.

The polynucleotide or vector of the invention may be delivered by any suitable method. For example, when the polynucleotide may be in a viral vector or the vector may be a viral vector and delivered by transduction.

Any suitable dose of the polynucleotide or vector may be used. For example, the vector may be delivered at a MOI of about 10⁴ to 10⁵ vg/cell, optionally about 10⁴ vg/cell.

Delivery of a p53 Inhibitor and/or HDR Enhancer

The method may further comprise a step of delivering a p53 inhibitor and/or HDR enhancer. The p53 inhibitor and/or HDR enhancer may be delivered simultaneously. The p53 inhibitor and/or HDR enhancer may be delivered simultaneously with or after the RNA-guided nuclease and/or the guide RNA(s).

As used herein, a “p53 inhibitor” is a substance which inhibits activation of the p53 pathway. The p53 pathway plays a role in regulation or progression through the cell cycle, apoptosis, and genomic stability by means of several mechanisms including: activation of DNA repair proteins, arrest of the cell cycle; and initiation of apoptosis. Inhibition of this p53 response by delivery during editing has been shown to increase hematopoietic repopulation by treated cells (Schiroli, G. et al. 2019. Cell Stem Cell 24, 551-565). Suitably, the p53 inhibitors is a dominant-negative p53 mutant protein, e.g. GSE56.

GSE56 May have the Amino Acid Sequence:

(SEQ ID NO: 152)
CPGRDRRTEEENFRKKEEHCPELPPGSA
KRALPTSTSSSPQQKKKPLDGEYFTLKI
RGRERFEMFRELNEALELKDARAAEESG
DSRAHSSYPK

In one embodiment, the p53 dominant negative peptide is a variant of GSE56 comprising 1, 2, 3, 4, 5, 6, 7, 8, 9 or 10 amino acid substitutions, additions or deletions, while retaining the activity of GSE56, for example in reducing or preventing p53 signalling.

In one embodiment, the p53 dominant negative peptide comprises an amino acid sequence having at least 70%, 75%, 80%, 85%, 90%, 95%, 96%, 97%, 98% or 99% identity to SEQ ID NO: 152.

As used herein, an “HDR enhancer” is a substance that is capable of improving HDR efficiency in HSCs, HPCS, and/or LPCs or CD34+ cells. HDR is constrained in long-term-repopulating HSCs. Any suitable HDR enhancer may be used, for example as described in Ferrari, S., et al., 2020. Nature Biotechnology, pp. 1-11. Suitably, the HDR enhancer is the adenovirus 5 E4orf6/7 protein. Adenovirus 5 E4orf6/7 proteins may be as disclosed in WO 2020/002380 (incorporated herein by reference).

The p53 inhibitor and the HDR enhancer may be delivered by any suitable method. The p53 inhibitor and/or the HDR enhancer may be transiently expressed, for example the p53 inhibitor and/or the HDR enhancer may delivered via mRNA. The p53 inhibitor and the HDR enhancer may be delivered by separate mRNAs or on a single mRNA encoding a fusion protein, optionally with a self-cleaving peptide (e.g. P2A). Any suitable dose of the p53 inhibitor and/or the HDR enhancer may be used, for example mRNA be delivered at a concentration of about 10-1000 μg/ml, about 50-500 μg/ml, or about 150 μg/ml.

In some embodiments, step (b) comprises:
(b1) delivering a RNA-guided nuclease and a guide RNA(s) of the invention, optionally preassembled in the form of a RNP complex by electroporation;
(b2) optionally, delivering a p53 inhibitor and/or a HDR enhancer; and
(b3) delivering a polynucleotide or vector of the invention by transduction to provide a gene-edited population of cells.

### Culturing the Gene-Edited Cell or Population of Gene-Edited Cells

The method may further comprise a step of culturing the population of gene-edited cells. This may be an expansion step, i.e. the method may further comprises a step of expanding the population of gene-edited cells.

The culturing step (e.g. expansion step) may be carried out using any suitable conditions.

During the culturing step (e.g. expansion step) the population of cells may be seeded at a concentration of about 1×10⁵ cells/ml to about 10×10⁵ cells/ml, e.g. about 2×10⁵ cells/ml, or about 5×10⁵ cells/ml. Suitably, the culturing step (e.g. expansion step) is for at least one day, or one to five days. For example, the culturing step (e.g. expansion step) may be for about one day. Suitably, the population of cells are cultured in a 5% CO₂ humidified atmosphere at 37° C.

Any suitable culture medium may be used. For example, commercially available medium such as StemSpan medium may be used, which contains bovine serum albumin, insulin, transferrin, and supplements in Iscove's MDM. The culture medium may be supplemented with one or more antibiotic (e.g. penicillin, streptomycin). The culturing step (e.g. expansion step) may be carried out in the presence in of one or more cytokines and/or growth factors.

In some embodiments, step (b) comprises:
(b1) delivering a RNA-guided nuclease and a guide RNA(s) of the invention, optionally preassembled in the form of a RNP complex by electroporation;
(b2) optionally, delivering a p53 inhibitor and/or a HDR enhancer;
(b3) delivering a polynucleotide or vector of the invention by transduction to provide a gene-edited population of cells; and
(b4) culturing (e.g. expanding) the gene-edited population of cells.

### Methods of Treatment

In one aspect the present invention provides a method of treating a subject using polynucleotides, vectors, guide RNAs, kits, compositions, gene-editing systems, cells and/or populations of cells of the present invention. Suitably, the method of treating a subject may comprise administering a cell or population of cells of the present the invention.

In a related aspect the present invention provides a polynucleotide, vector, guide RNA, kit, composition, gene-editing system, cell and/or populations of cells of the present invention for use as a medicament. Suitably, the cell or population of cells of the present the invention may be used as a medicament.

In a related aspect, the present invention provides use of a polynucleotide, vector, guide RNA, kit, composition, gene-editing system, cell and/or populations of cells of the present invention for the manufacture of a medicament. Suitably, the cell or population of cells of the present the invention may be used for the manufacture of a medicament.

Suitably, a method of treating a subject may comprise:
(a) providing a cell or a population of cells;
(b) using a kit, composition, and/or gene-editing system described herein to obtain a gene-edited cell or a population of gene-edited cells; and
(c) administering the population of gene-edited cells to the subject.

For example, a method of treating a subject may comprise:
(a) providing a cell or a population of cells;
(b) delivering an RNA-guided nuclease, a guide RNA, and/or a polynucleotide or vector of the present invention to the cell or population of cells to obtain a gene-edited cell or a population of gene-edited cells; and
(c) administering the population of gene-edited cells to the subject.

Steps (a) and (b) may be identical to the steps described in the section above.

Suitably, the cell of population of cells may be isolated and/or enriched from the subject to be treated, e.g. the population of cells may be an autologous population of CD34+ cells. Suitably, the population of cells are isolated from (mobilised) peripheral blood or cord blood of the subject to be treated and subsequently enriched (e.g. by FACS and/or magnetic bead sorting).

The subject may be immunocompromised and/or the disease to be treated may be an immunodeficiency, i.e. the medicament may be for treating an immunodeficiency. As used herein, an “immunodeficiency” is a disease in which the immune system's ability to fight infectious disease and cancer is compromised or entirely absent. A subject who has an immunodeficiency is said to be “immunocompromised”. An immunocompromised person may be particularly vulnerable to opportunistic infections, in addition to normal infections that could affect everyone.

### RAG Deficient-Immunodeficiency

The subject may have RAG deficiency, e.g. a RAG1 deficiency. A RAG1 deficiency may be due to a loss-of-function mutation in the RAG1 gene, optionally a loss-of-function mutation in the RAG1 exon 2.

The immunodeficiency may be a RAG deficient-immunodeficiency. As used herein, a “RAG deficient-immunodeficiency” is an immunodeficiency characterised by loss of RAG1/RAG2 activity. A RAG deficient-immunodeficiency may, for example be caused by a mutation in RAG genes.

Suitably, the RAG deficient-immunodeficiency may be a RAG1 deficiency. A RAG1 deficiency may be due to a loss-of-function mutation in the RAG1 gene, optionally a loss-of-function mutation in the RAG1 exon 2.

Mutations of the RAG genes in humans are associated with distinct clinical phenotypes, which are characterized by variable association of infections and autoimmunity. In some cases, environmental factors have been shown to contribute to such phenotypic heterogeneity. In humans, RAG1 deficiency can cause a broad spectrum of phenotypes, including T- B-SCID, Omenn syndrome (OS), atypical SCID (AS) and combined immunodeficiency with granuloma/autoimmunity (CID-G/AI). (Notarangelo, L. D., et al., 2016. Nature Reviews Immunology, 16(4), pp. 234-246 and Delmonte, O. M., et al., 2018. Journal of clinical immunology, 38(6), pp. 646-655).

In some embodiments, the RAG deficient-immunodeficiency is T⁻ B⁻ SCID, Omenn syndrome, atypical SCID, or CID-G/AI.

Severe combined immunodeficiency (SCID) comprises a heterogeneous group of disorders that are characterized by profound abnormalities in the development and function of T cells (and also B cells in some forms of SCID), and are associated with early-onset severe infections. This condition is inevitably fatal early in life, unless immune reconstitution is achieved, usually with HSCT. Following the introduction of newborn screening for SCID in the United States, it has become possible to establish that RAG mutations account for 19% of all cases of SCID and SCID-related conditions, and are a prominent cause of atypical SCID and Omenn syndrome in particular. (Notarangelo, L. D., et al., 2016. Nature Reviews Immunology, 16(4), pp. 234-246).

In 1996, RAG mutations were identified as the main cause of T-B-SCID with normal cellular radiosensitivity. A distinct phenotype characterizes Omenn syndrome, which was first described in 1965. These patients manifest early-onset generalized erythroderma, lymphadenopathy, hepatosplenomegaly, eosinophilia and severe hypogammaglobulinaemia with increased IgE levels, which are associated with the presence of autologous, oligoclonal and activated T cells that infiltrate multiple organs. In some patients with hypomorphic RAG mutations, a residual presence of autologous T cells was demonstrated without clinical manifestations of Omenn syndrome. This condition is referred to as ‘atypical’ or ‘leaky’ SCID. A distinct SCID phenotype involving the oligoclonal expansion of autologous γδ T cells (referred to here as γδ T+ SCID) has been reported in infants with RAG deficiency and disseminated cytomegalovirus (CMV) infection. (Notarangelo, L. D., et al., 2016. Nature Reviews Immunology, 16(4), pp. 234-246).

Whereas SCID, atypical SCID and Omenn syndrome are inevitably fatal early in life if untreated, several forms of RAG deficiency with a milder clinical course and delayed presentation have been reported in recent years. In particular, the occurrence of CID-G/AI was reported in three unrelated girls with RAG mutations who manifested granulomas in the skin, mucous membranes and internal organs, and had severe complications after viral infections, including B cell lymphoma. Following this description, several other cases of CID-G/AI with various autoimmune manifestations (such as cytopaenias, vitiligo, psoriasis, myasthenia gravis and Guillain-Barré syndrome) have been reported. (Notarangelo, L. D., et al., 2016. Nature Reviews Immunology, 16(4), pp. 234-246).

Additional phenotypes that are associated with RAG deficiency include idiopathic CD4⁺ T cell lymphopaenia, common variable immunodeficiency, IgA deficiency, selective deficiency of polysaccharide-specific antibody responses, hyper-IgM syndrome and sterile chronic multifocal osteomyelitis. (Notarangelo, L. D., et al., 2016. Nature Reviews Immunology, 16(4), pp. 234-246).

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1—RAG1 Gene Exonic Strategies and RAG1 Gene Replacement Strategies

### Exonic Qenome Editing Strategies

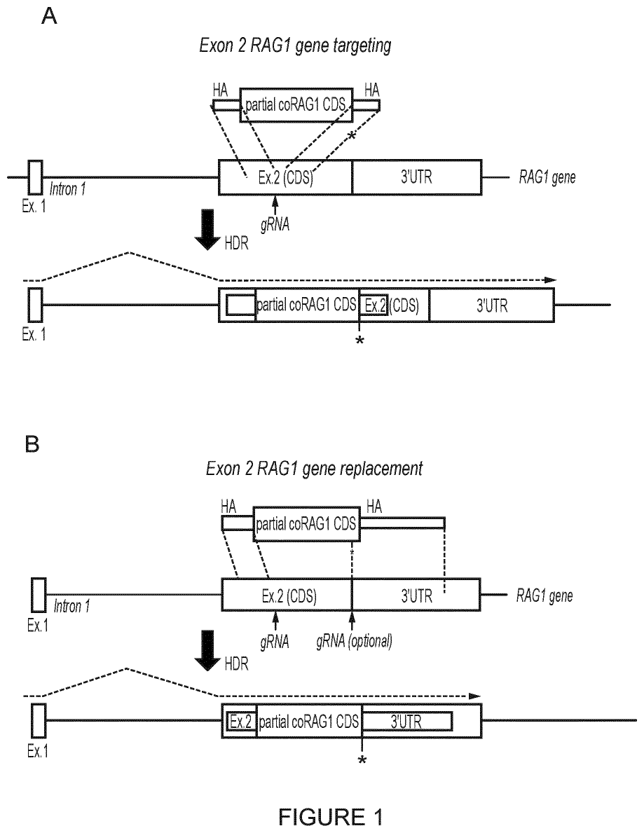

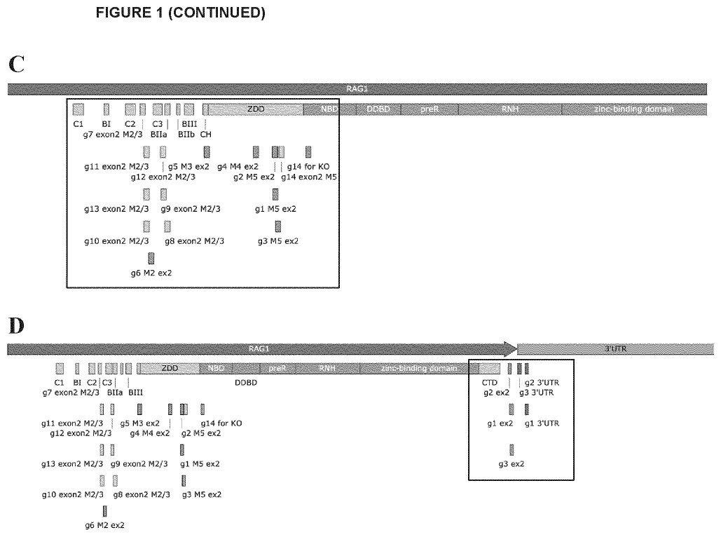

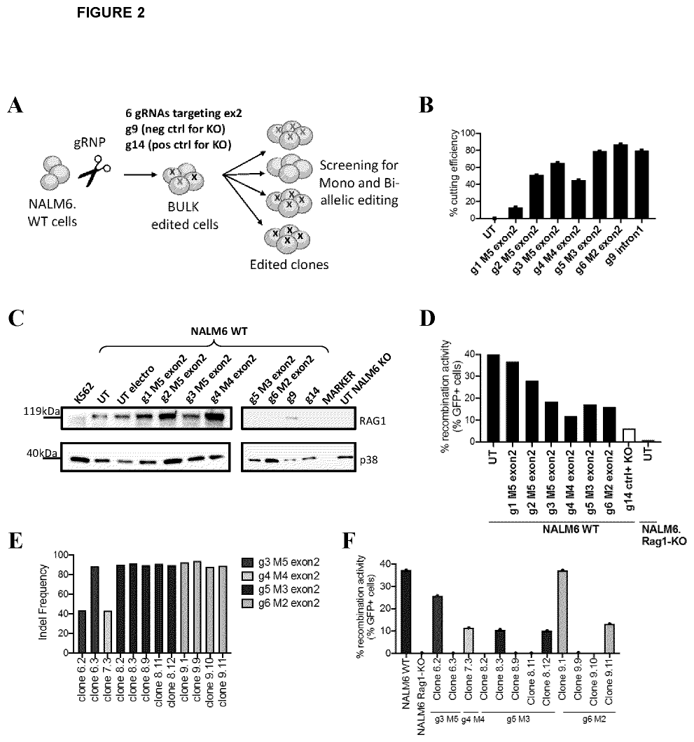

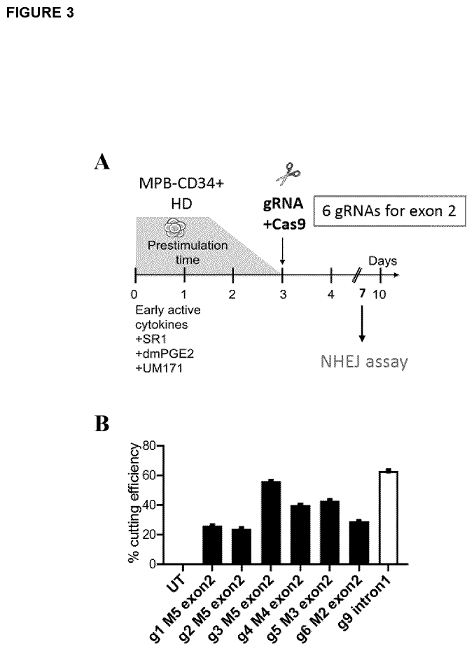

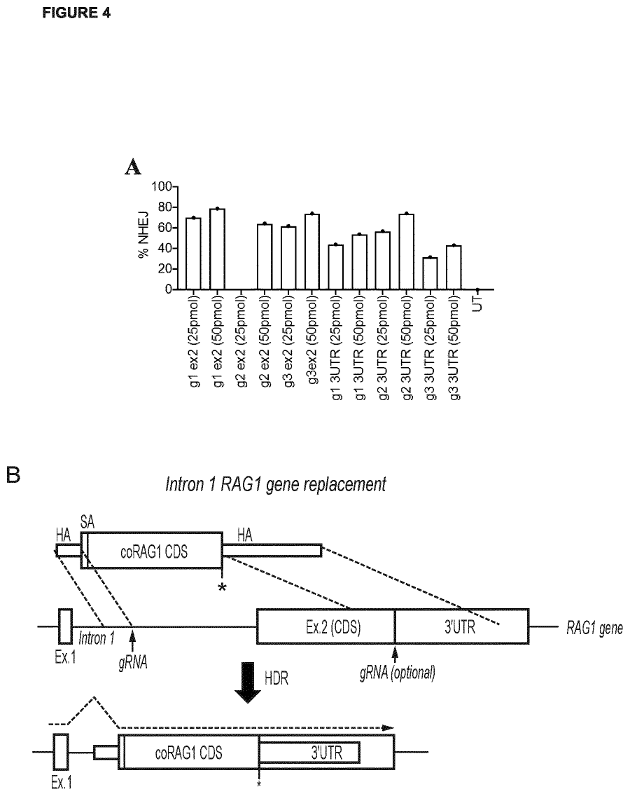

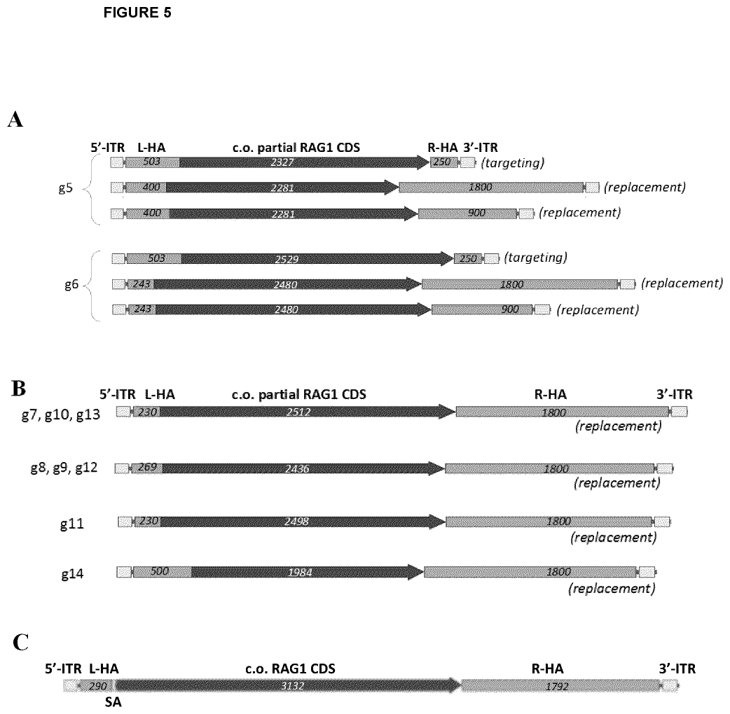

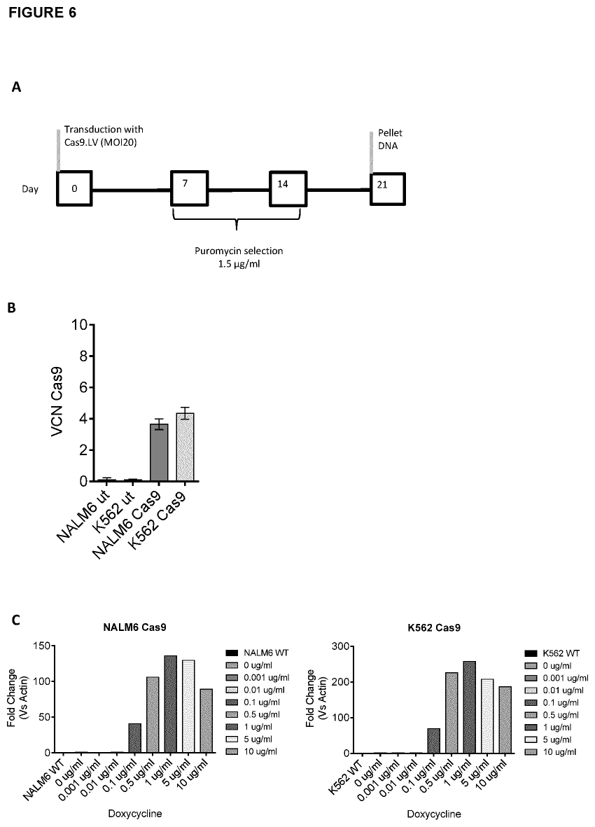

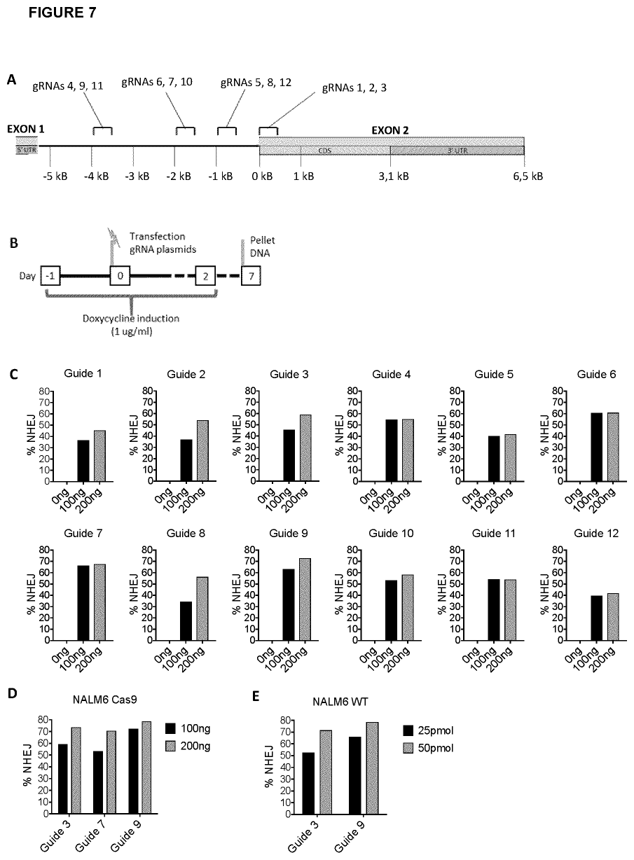

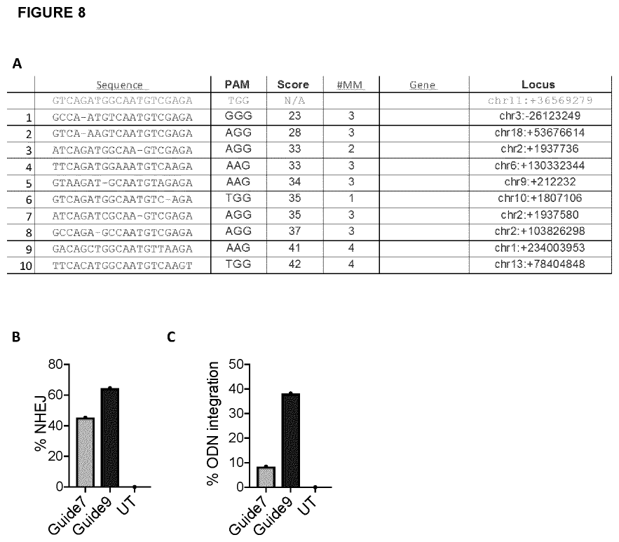

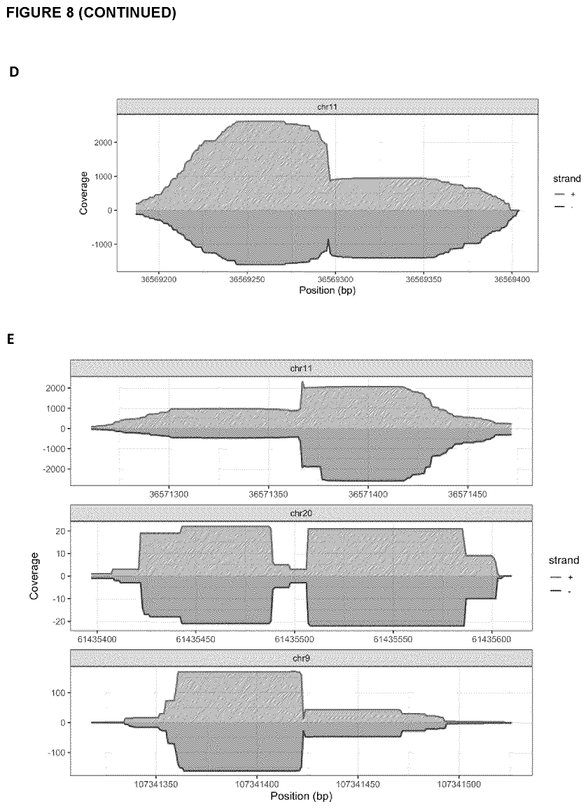

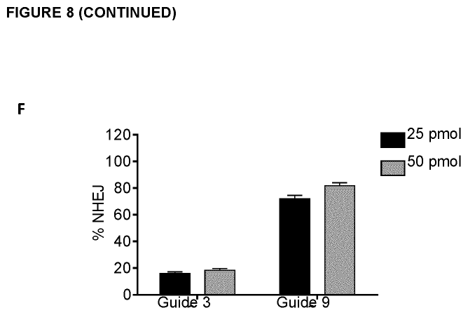

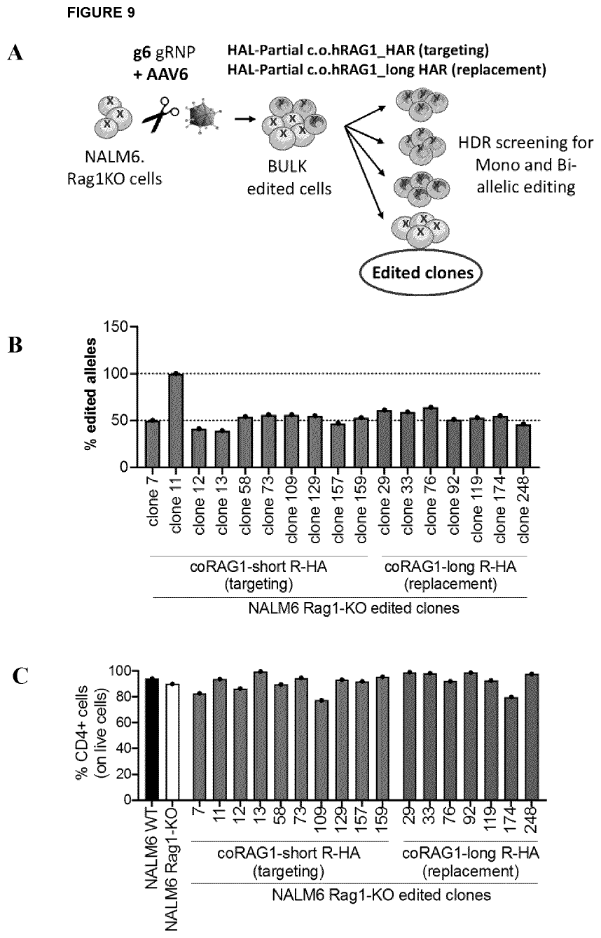

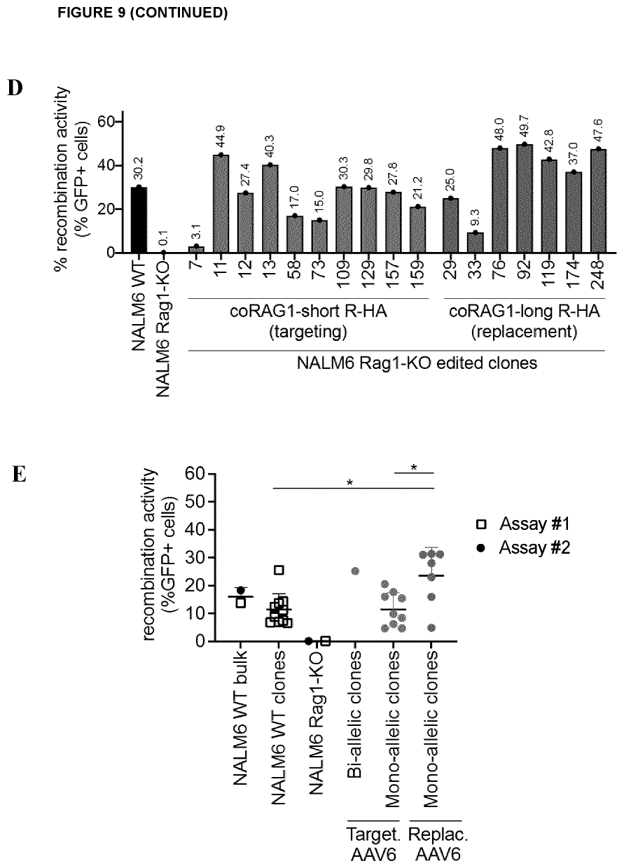

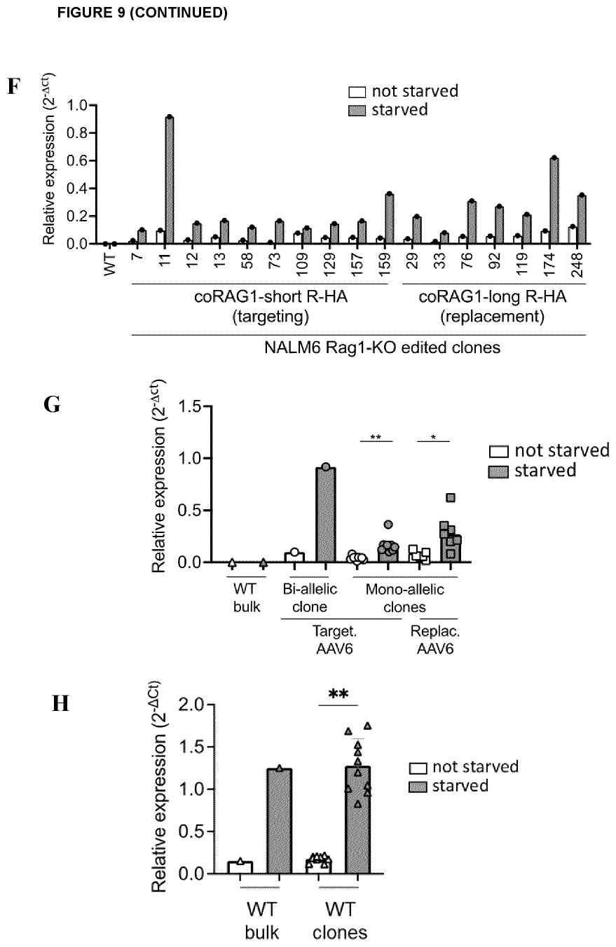

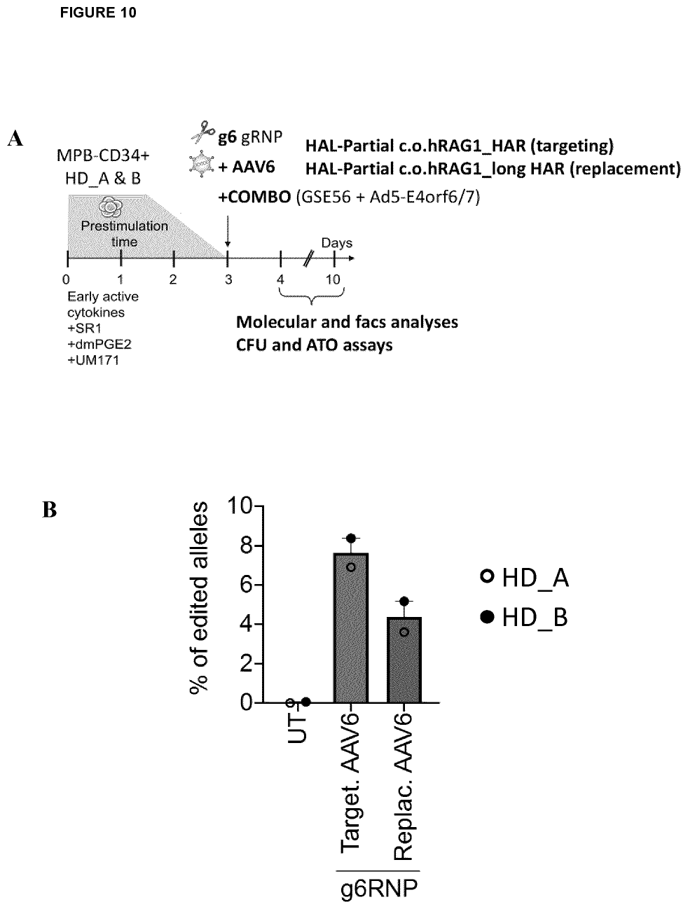

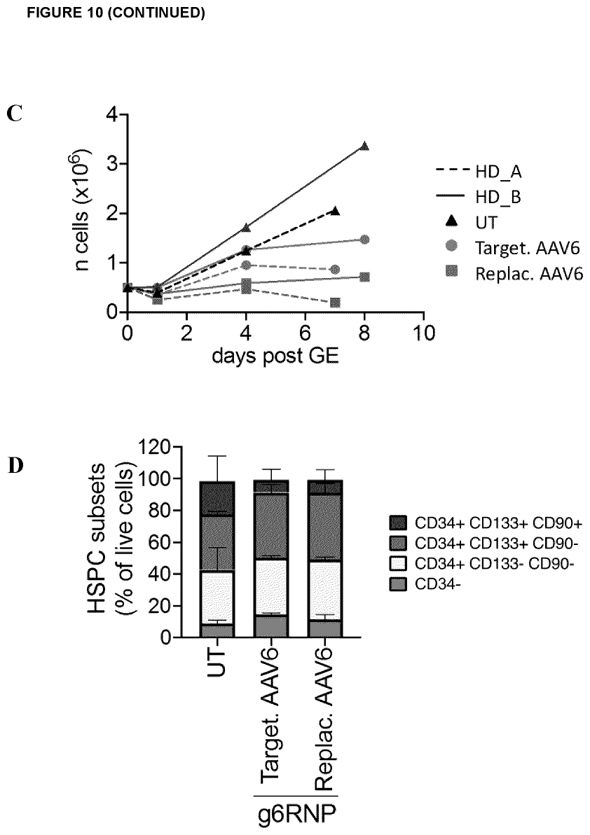

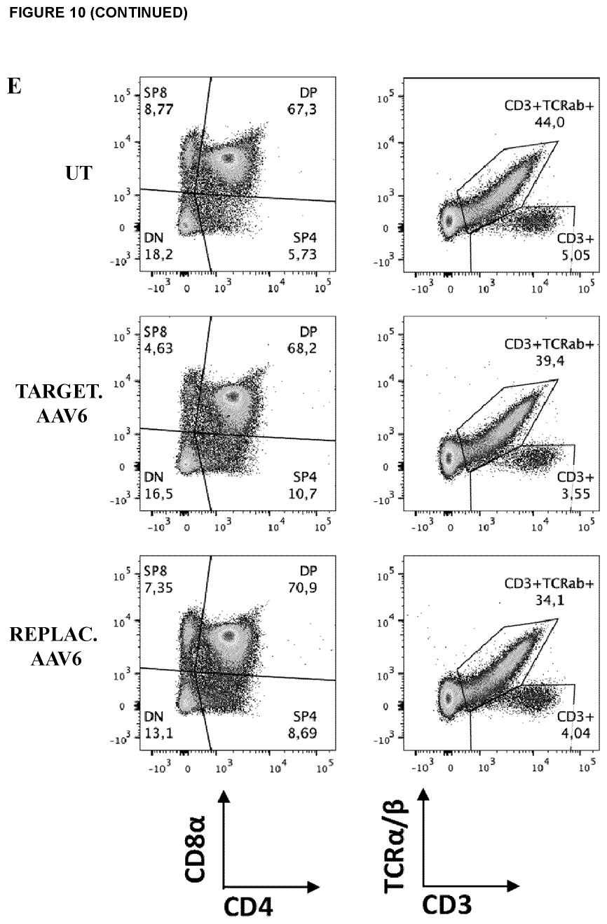

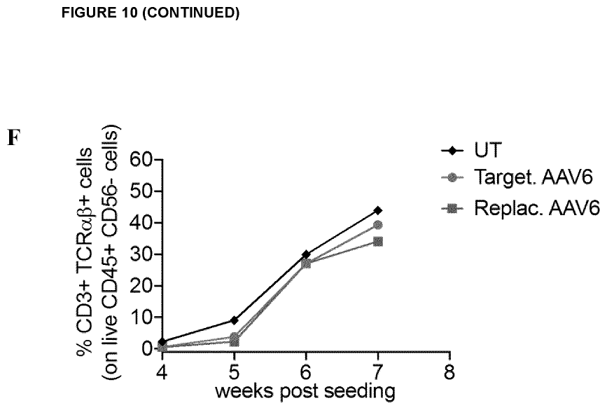

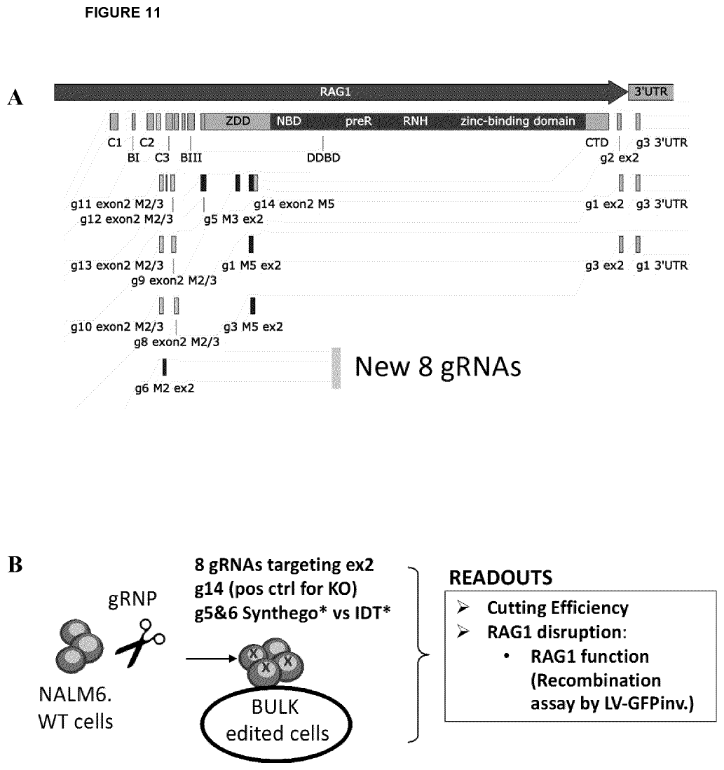

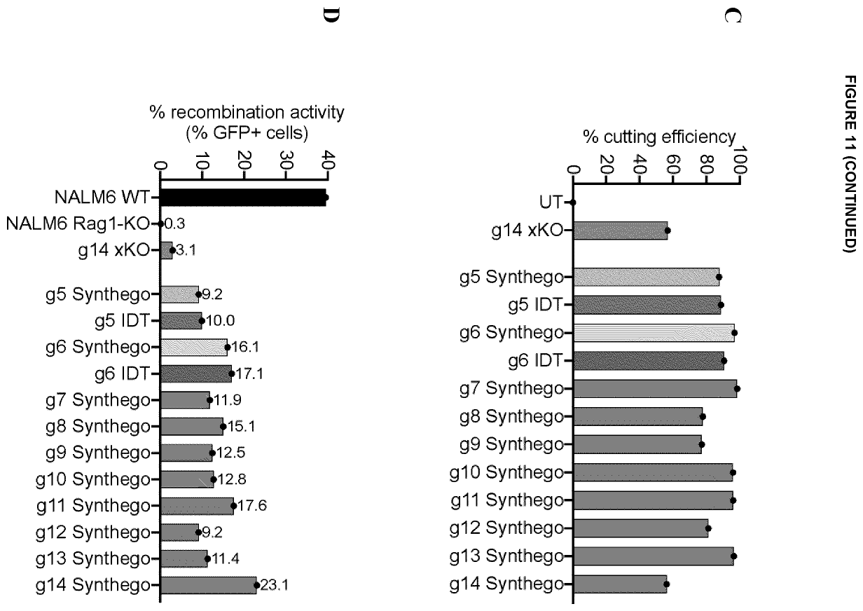

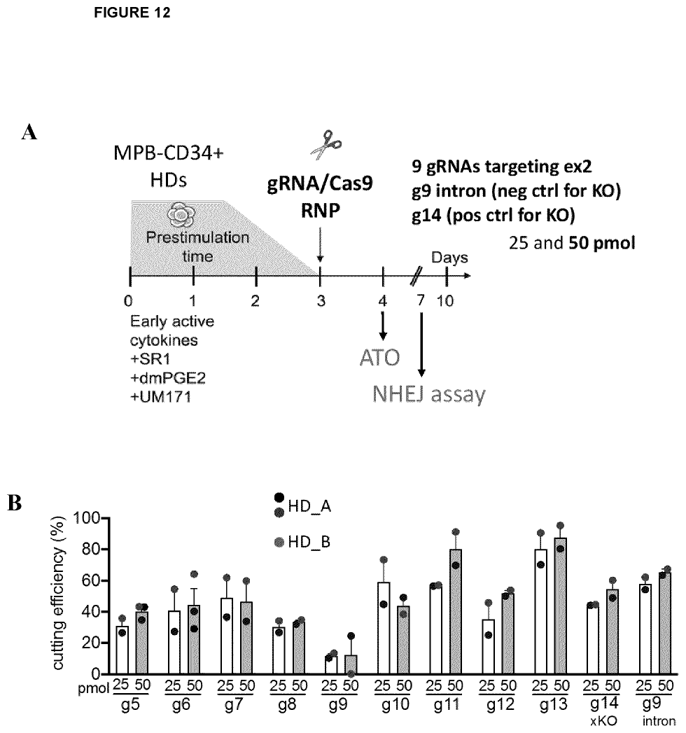

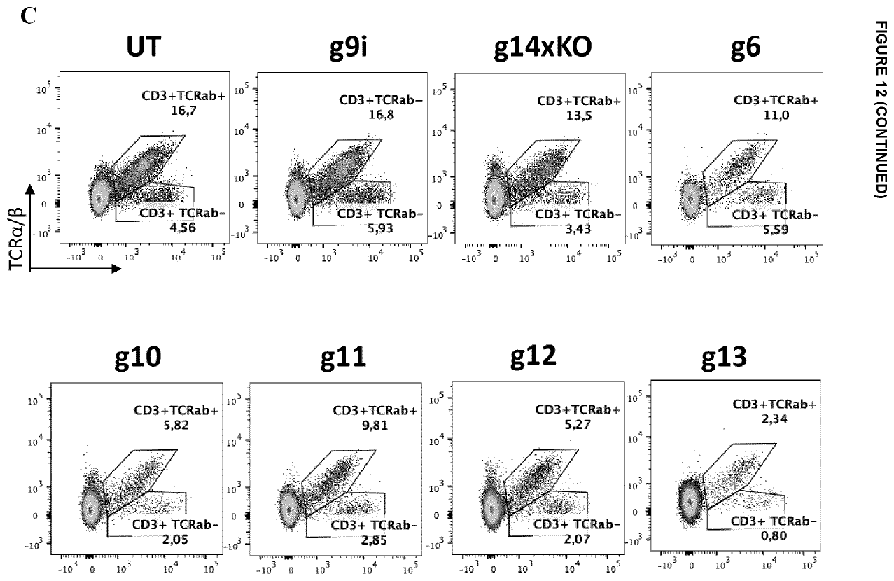

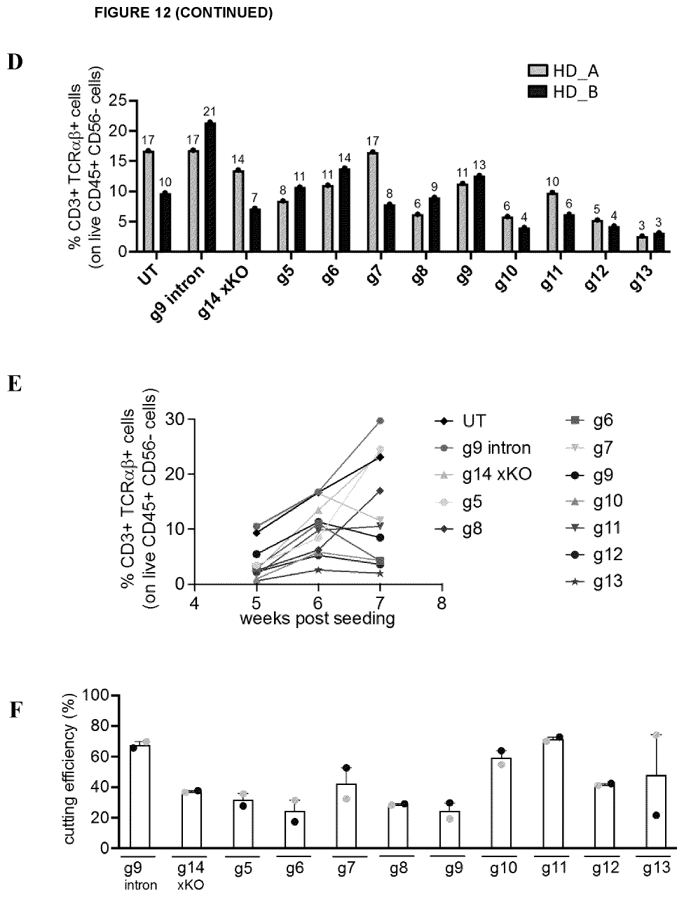

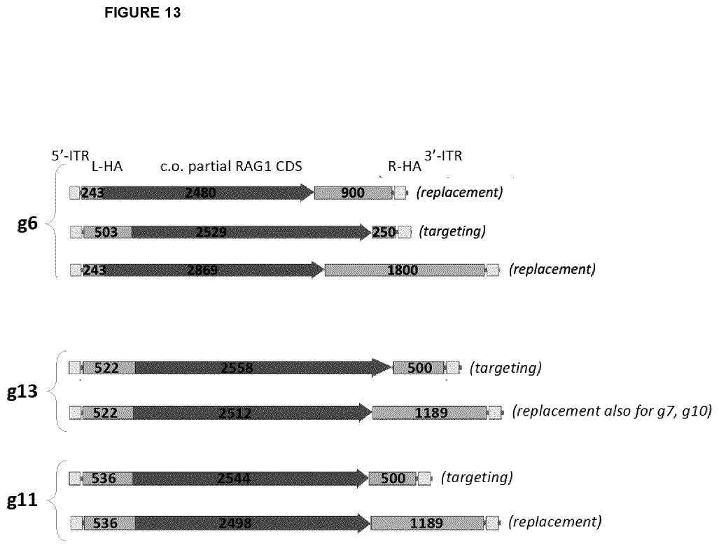

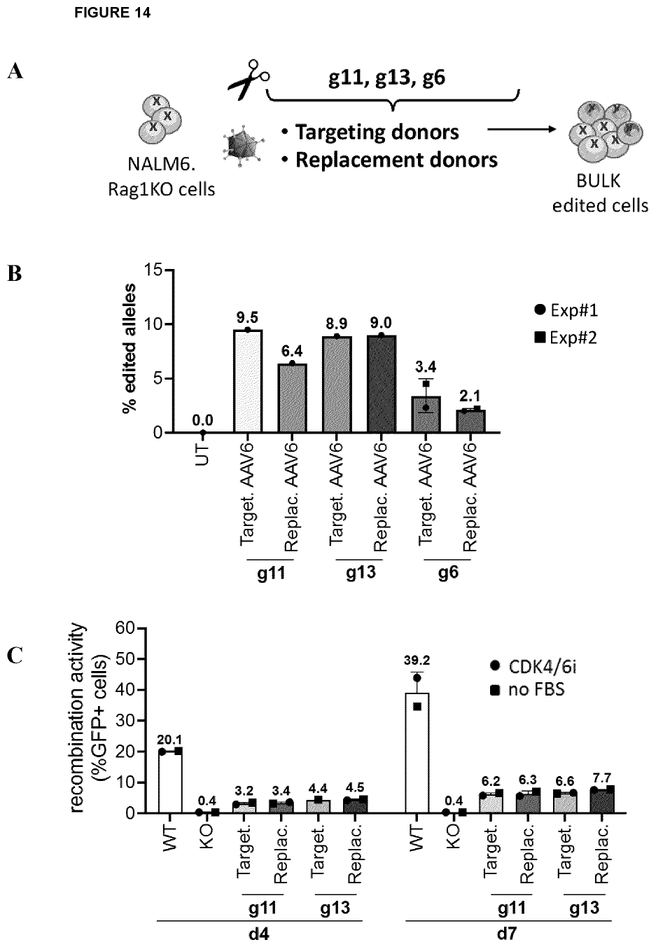

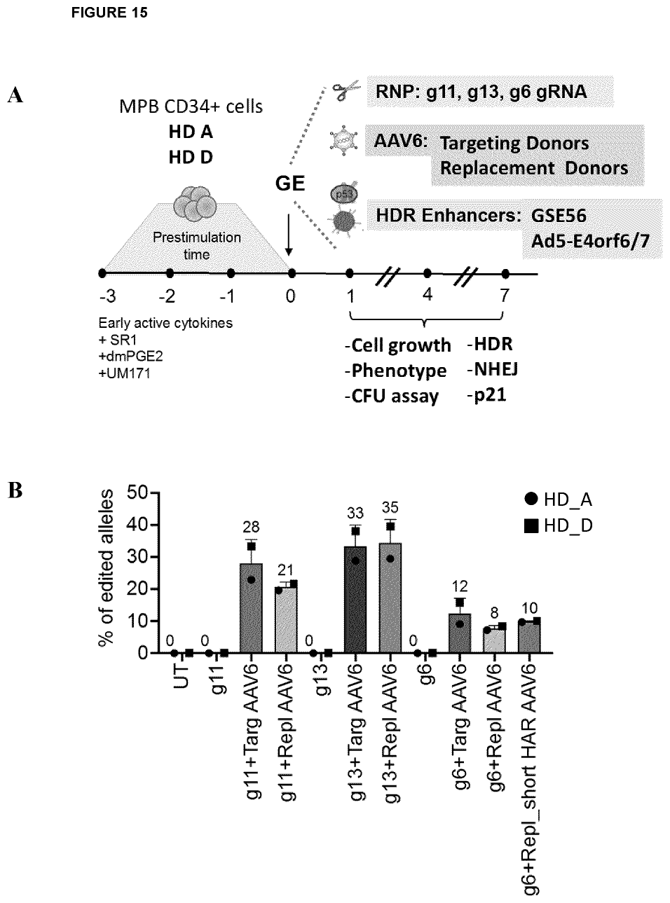

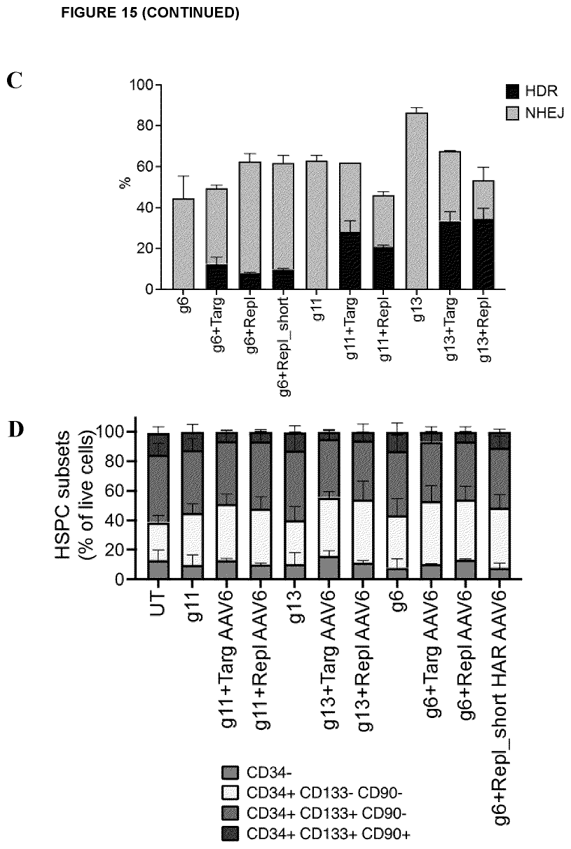

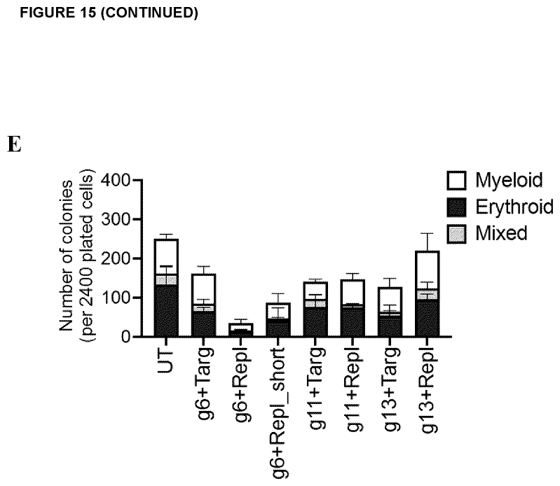

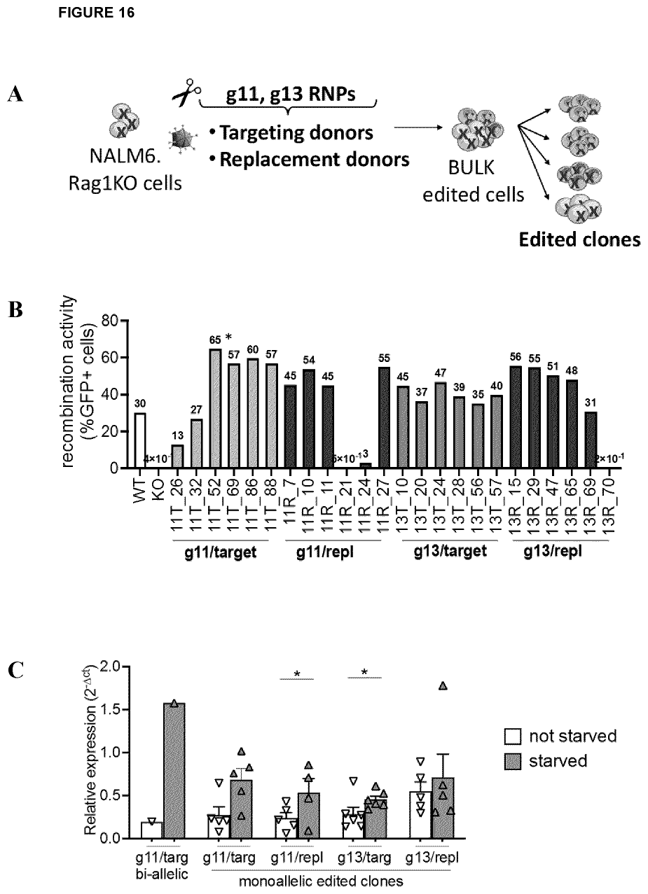

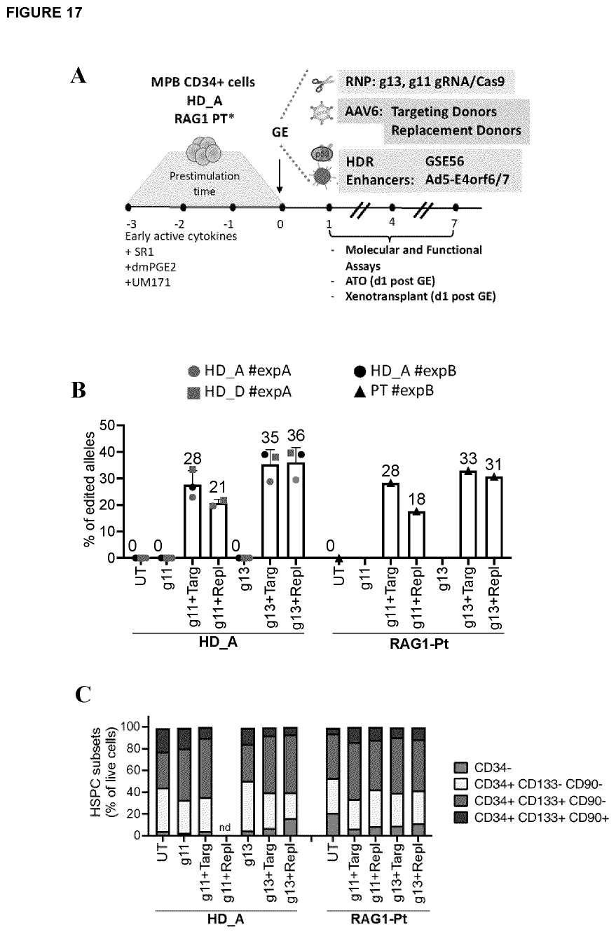

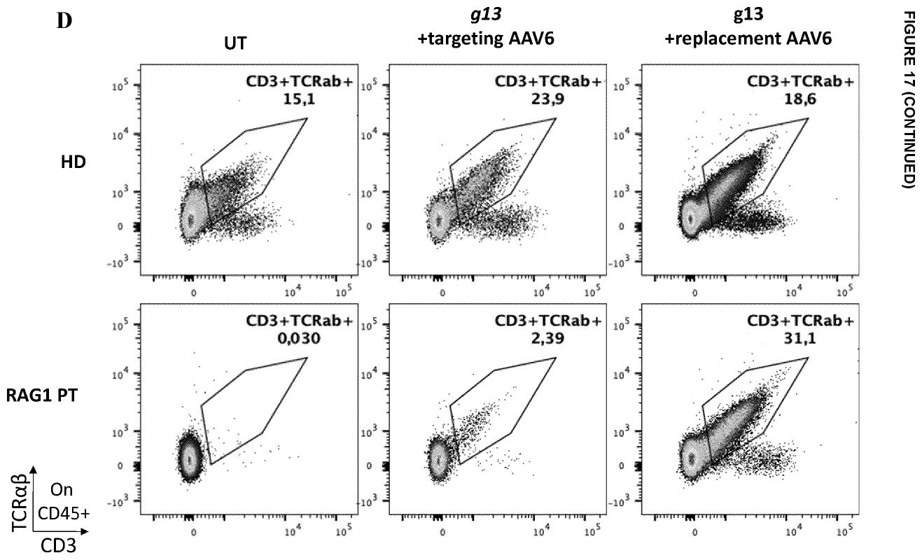

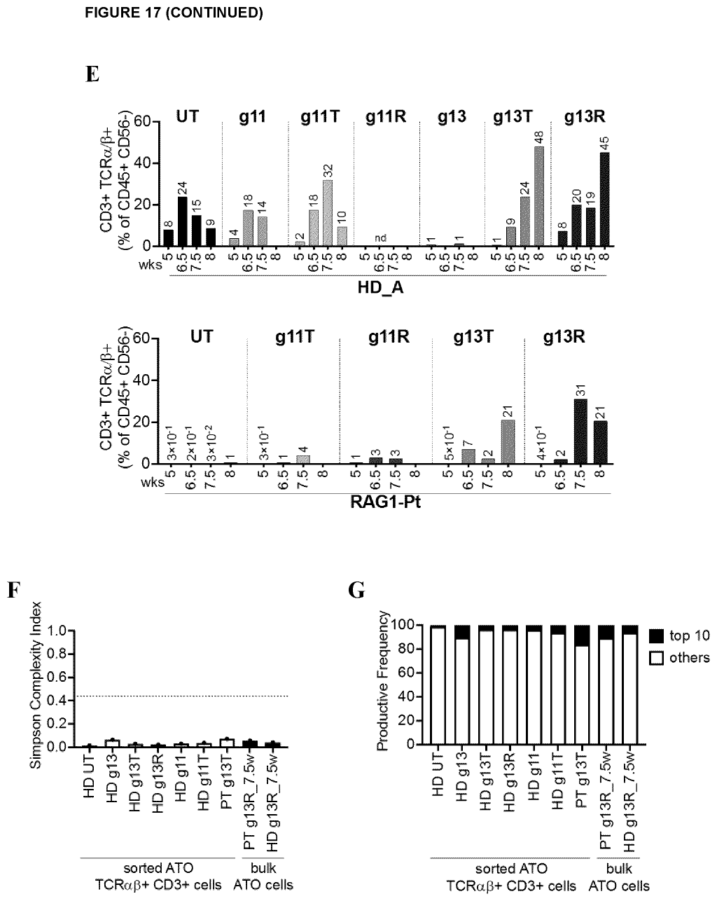

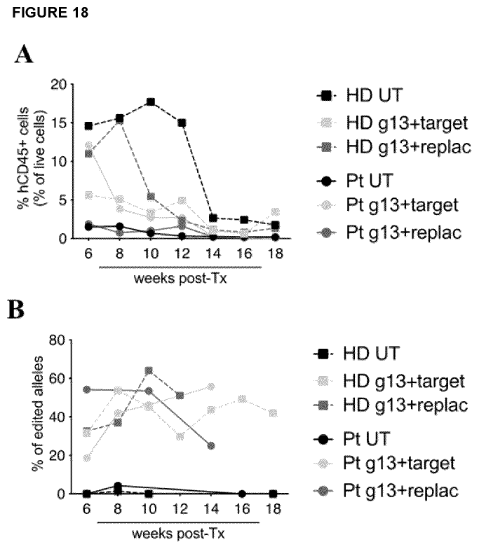

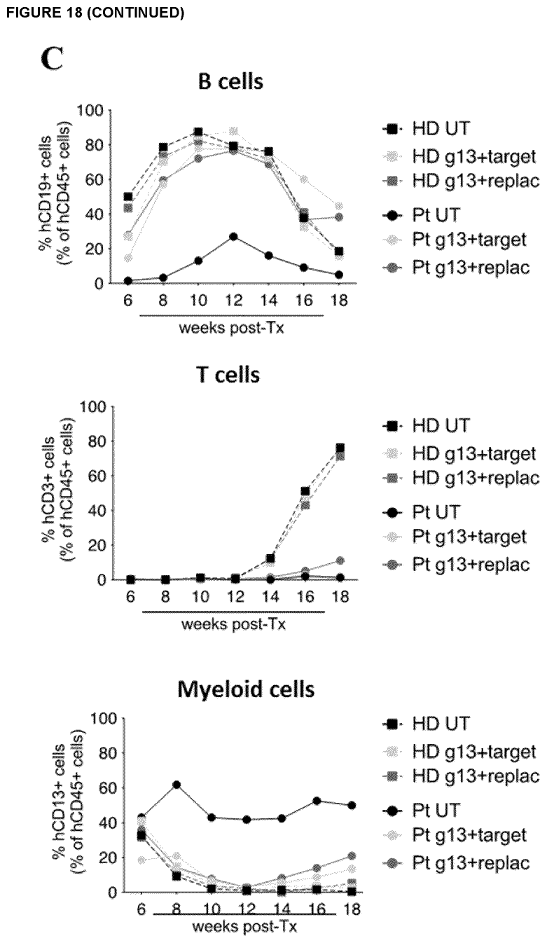

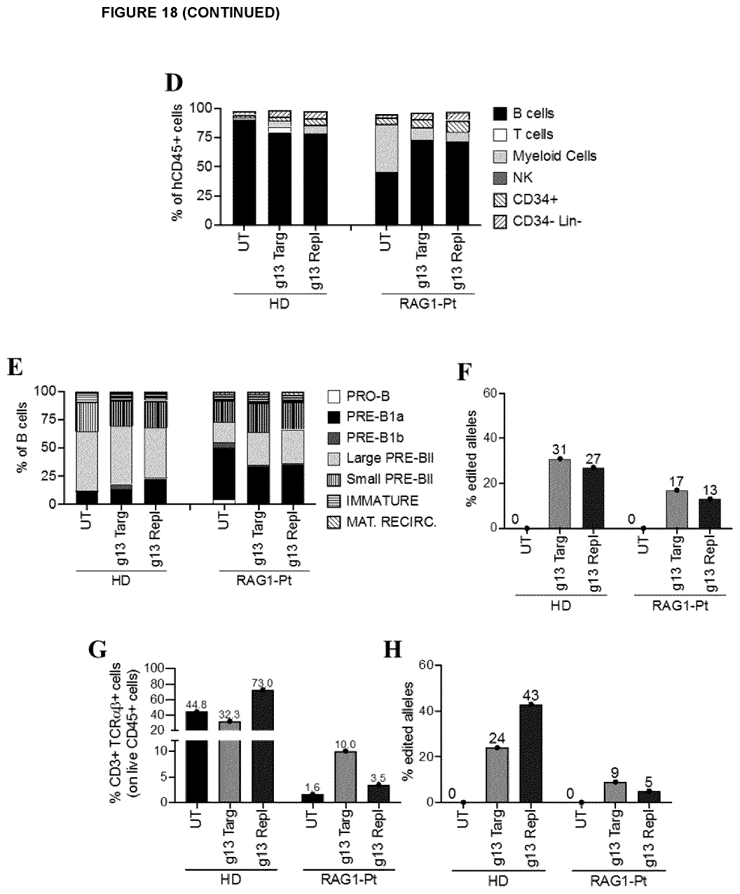

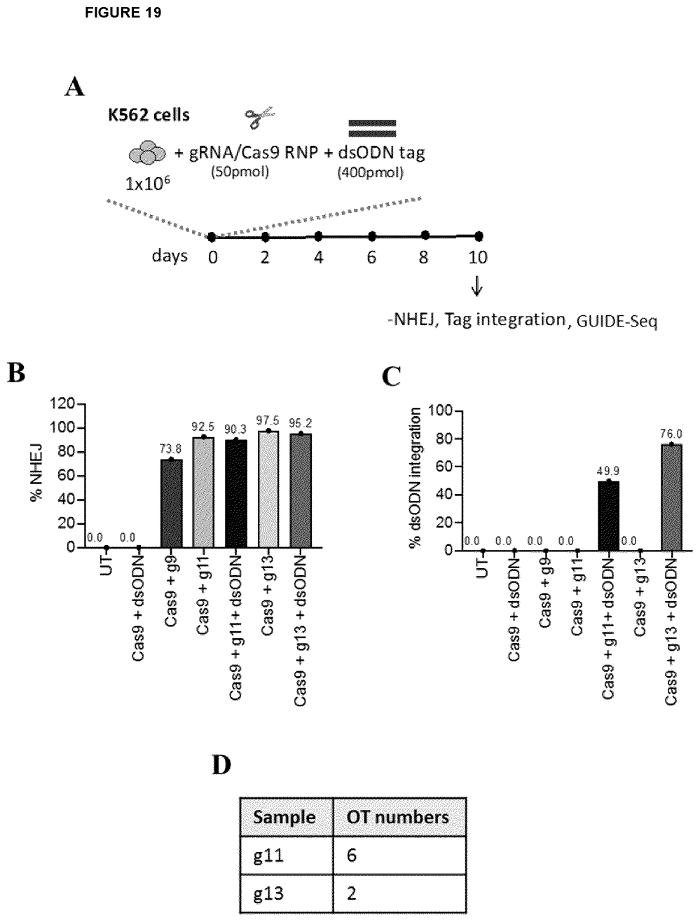

Two exonic strategies have been developed to correct the human RAG1 gene:
1) The “exon 2 RAG1 gene targeting” is based on the targeting of the second RAG1 exon (FIG. 1A). Following the DNA double-stand break (DSB), non-homologous end joining (NHEJ) repair machinery would disrupt the endogenous RAG1 gene while alleles edited by homology directed repair (HDR) will allow RAG1 correction, increasing the selective advantage of corrected cells over the RAG1 deficient cells. The corrective donor carries a codon optimized RAG1 (coRAG1) partial coding sequence (CDS) in frame with the upstream portion of the endogenous RAG1 and homology arms close to the exonic cutting site, downstream from the endogenous splice acceptor. This strategy will preserve the intronic region, maintain the 3′UTR regulation and promote the selective advantage of corrected cells. This strategy will allow precise gene correction, maintaining the genomic region upstream of the exon 2 and the 3′UTR regulatory region, while providing a more selective advantage of the corrected gene edited cells over the mutated CD34⁺ cells carrying hypomorphic RAG1 mutations.
2) The “exon 2 RAG1 gene replacement” is based the same rationale. However, the corrective template is designed to include, along with the left homology arm flanking the exonic gRNA cutting site, a right homology arm which is homologous to a distant downstream sequence, that includes the initial part of the 3′UTR (FIG. 1B). Optionally, a second gRNA cutting just upstream the right homology arm can be used in combination with the first gRNA. The corrective donor carries a partial coRAG1 CDS in frame with the upstream portion of the endogenous RAG1 and preferentially a long homology sequence specific for part of the 3′UTR to favor HDR and gene replacement. Following the DNA DSB, the partial coRAG1, delivered by AAV6 or IDLV (or any other vector) replaces the excised endogenous RAG1 CDS.

### Guide RNA Selection for RAG1 Exonic Strategies

While RAG1 null mutations prevent the development of lymphocytes, the majority of RAG1 mutations described in literature impair but not abolish the V(D)J recombination activity leading to the generation of lymphoid progenitors that may compete with corrected T and B cell progenitors in central niches (Delmonte O M, et al. Blood. 2020; 135(9):610-9). To improve the selective advantage of corrected cells over the hypomorphic ones, we designed the “exon 2 RAG1 gene targeting” and the “exon 2 RAG1 gene replacement” strategies (FIG. 1A-B) to disrupt the endogenous RAG1 gene by NHEJ and correct it by HDR.

To select a gRNA specific for RAG1 exon 2 able to disrupt the endogenous RAG1 gene, we considered that nonstandard ATG are present at the N-terminal of RAG1 gene and may be used as alternative start sites for re-initiating translation process and producing RAG1 truncated protein with decreased recombination activity (Santagata S, et al. Proc Natl Acad Sci. 2000 Dec. 19; 97(26):14572-7). Thus, to achieve the complete RAG1 inactivation we designed and synthetized the following gRNAs targeting the last internal nonstandard Methionines (M) at 5′ of RAG1 (FIG. 1C) (PAM sequences are highlighted in bold):

g1 M5 ex2 RAG1:
TTGCTGGACATTTCACCATC AGG
g2 M5 ex2 RAG1:
TGCTGGACATTTCACCATCA GGG
g3 M5 ex2 RAG1:
TCCAGCAAAAGAGTGCAATG AGG
g4 M4 ex2 RAG1:
AAGCATGGATATCGGCAAGA GGG
g5 M3 ex2 RAG1:
AAGATGTATCTTACTGCAGT TGG
g6 M2 ex2 RAG1:
CGAGGAACGTGACCATGGAG TGG

These 6 gRNAs were tested for NHEJ efficiency and RAG1 disruption in NALM6-WT cells, which constitutively express RAG1.

Each gRNA was delivered into NALM6-WT cells as an in vitro preassembled RNPs (50 pmol/well) by electroporation (FIG. 2A). We assessed on bulk edited cells: the cutting efficiency (by a T7-mediated NHEJ assay), the protein production (by Western Blot, WB) and the RAG1-mediated recombination activity (by the transduction with a LV carrying an inverted GFP cassette). Ten days after the editing, NALM6 cells were collected, and DNA was extracted to assess the cutting efficiency of each gRNA (FIG. 2B). As internal control we used a gRNA (g9) targeting the intron 1 of RAG1 gene, which does not alter its open reading frame and was previously tested on NALM6-WT cells. “g5 M3 ex2” and “g6 M2 ex2” gRNAs (named g5 and g6 hereafter) showed a high cutting efficiency with values (79% and 87%, respectively) comparable to the control gRNA (80%) (FIG. 2B).

To verify the RAG1 disruption, we performed WB assay on protein lysates of bulk edited NALM6-WT cells. As positive controls, we used NALM6-WT cells (which constitutively expressed RAG1): i) untreated (UT), ii) electroporated in absence of gRNA (electro), or iii) edited with a gRNA targeting the intron (g9). As negative controls, we used protein lysates of: i) untreated K562 cells (which do not express RAG1), ii) NALM6-WT cells edited by g14 gRNA which leads to RAG1 protein disruption because it targets the RAG1 catalytic core, and iii) untreated NALM6-RAG1.KO cell line previously generated in our lab by editing NALM6-WT with g14 (FIG. 2C). We observed the absence of RAG1 protein in NALM6-WT cells edited by g5 and g6 despite the presence of the housekeeping protein p38, suggesting the RAG1 disruption (FIG. 2C).

The RAG1 inactivation was further evaluated in terms of function. To this aim, bulk edited NALM6 cells were transduced with LV carrying an inverted GFP cassette which is flanked by the Recombination Signal Sequences (RSS) specifically recognized by the RAG1/RAG2 complex. If functional, RAG1 recognizes and binds the RSS and recombines the GFP cassette, which will be placed in the correct orientation resulting in the expression of GFP. Thus, the percentage of GFP+ cells, analyzed by flow cytometry, is indicative of the RAG1-recombanition activity (Liang H E, et al. Immunity. 2002; Bredemeyer A L, et al. Nature. 2006 Jul. 14; 442(7101):466-70; De Ravin S S, et al. Blood. 2010; 116(8):1263-71; and Lee Y N, et al. J Allergy Clin Immunol. 2014). Untreated NALM6-WT cells showed 40.1% recombination activity, in line with our historical data. “g1 M5 ex2” and “g2 M5 ex2” gRNAs induced a slight reduction of the recombination activity, while the other four gRNAs induced a 2-fold reduction of RAG1 recombination activity (FIG. 2D). We also observed a reduced recombination activity even in cells edited by g5 and g6 gRNAs (FIG. 2D).

To overcome the limitation of bulk cell analysis, edited cells were subcloned by single-cell plate sorting. We selected mono- or bi-allelic edited clones by DNA sequencing. The frequency of insertion and deletion (indel) on 30 edited clones was assessed by TIDE analysis (http://shinyapps.datacurators.nl/tide/). We selected two mono-allelic edited clones (clone 6.2 and clone 7.3) and ten bi-allelic edited clones (FIG. 2E). Preliminary data showed that the gRNAs were able to knock-out RAG1 completely abrogating the recombination activity in some clones edited by “g3 M5 ex2” (clone 6.3), g5 (clones 8.9 and 8.11) and g6 (clones 9.9 and 9.10) gRNAs (FIG. 2F).

We also tested these 6 gRNAs targeting RAG1 exon 2 for NHEJ efficiency in CD34⁺ hematopoietic stem and progenitor cells (HSPC). Hematopoietic stem and progenitor cells derived from mPB of HD were thawed at day 0 and prestimulated for three days seeding 0.5×10⁶ cells/ml in StemSpan enriched with cytokines (hTPO 100 ng/ml, hSCF 300 ng/ml, hFlt3-L 300 ng/ml, SR1 1 uM, UM171 35 nM, PGE2 10 uM). At day 3, gRNAs were delivered as an in vitro preassembled RNPs (50 pmol/well) by electroporation. Four days after the editing, cells were collected, and DNA was extracted to measure the cutting efficiency of each gRNA by performing the NHEJ assay (T7 mediated) (FIG. 3A). As internal control we used a gRNA (g9) previously tested on CD34+ cells. The cutting efficiency values ranged between 27% for “g2 M5 exon2” and 56% for “g3 M5 exon2” gRNA (FIG. 3B).

To further improve the cutting efficiency, we designed other 7 new gRNAs targeting the region between the second and the third Methionine (M2/3), the region targeted by g6 gRNA, and 1 new gRNA targeting the M5 (FIG. 1C) (PAM sequences are highlighted in bold):

g7 exon2 M2/3:
GTTTAGCAGTGCCCCATGTG AGG
g8 exon2 M2/3:
CTTCCTCTTGAGTCCCCGAC GGG
g9 exon2 M2/3:
ATCTGCAACACTGCCCGTCG GGG
g10 exon2 M2/3:
TCGGGAAGTAAACCTCACAT GGG
g11 exon2 M2/3:
CATGTGAGGTTTACTTCCCG AGG
g12 exon2 M2/3:
ACATCTGCAACACTGCCCGT CGG
g13 exon2 M2/3:
CGGGAAGTAAACCTCACATG GGG
g14 exon2 M5:
GTGCAATGAGGAGGTCAGTT TGG

These 8 gRNAs can also be tested for NHEJ efficiency in CD34+ HSPCs and in NALM6 cells. Moreover, to verify RAG1 disruption, the RAG1 expression (by RT-PCR/ddPCR), protein production (by WB) and recombination activity in NALM6 cells treated with various gRNAs can be assessed.

### Guide RNA Selection for RAG1 Replacement Strategies

The “exon 2 RAG1 gene replacement” strategy (FIG. 1B) can optionally exploit the co-electroporation of two gRNAs targeting the intron and a sequence downstream of the second exon. Therefore, the best performing gRNA selected in the group described above can be combined with a gRNA mapping the 3′ exonic region or the first nucleotides of the 3′UTR (PAM sequences are highlighted in bold) (FIG. 1D) (PAM sequences are highlighted in bold):

g1 ex2:
GAGAGTCCTCTATGCCTAAT GGG
g2 ex2:
AGGGGACCCATTAGGCATAG AGG
g3 ex2:
AGAGAGTCCTCTATGCCTAA TGG
g1 3′UTR:
AAGCCCTCAATGCAACCCAG AGG
g2 3′UTR:
AGCCCTCAATGCAACCCAGA GGG
g3 3′UTR:
TAGGGCAACCACTTATGAGT TGG

These six gRNAs have been tested in CD34+ cells at the doses of 25 and 50 pmol to assess the NHEJ efficiency by the T7 surveyor assay. The “g1 exon2” gRNA showed the highest cutting efficiency (FIG. 4A).

The “exon 2 RAG1 gene replacement” can be compared with the “intron 1 RAG1 gene replacement” strategy shown in FIG. 4B. Both strategies are based on the design of distant homology arms homologous to a downstream sequence including the initial part of the 3′UTR. The use of a long homology arm specific for part of the 3′UTR may favor HDR and gene replacement.

Optionally, a second gRNA cutting just upstream the right homology arm can be used in combination with the first selected gRNA, specific for the exonic or intronic strategy. In case of the intronic strategy (FIG. 4B), following the DNA DSB, the endogenous CDS is excised, and the corrective donor DNA is integrated into the intronic region by HDR, thanks to the presence of two homology arms flanking the corrective donor. The donor carries the splice acceptor (SA) sequence upstream the corrective DNA sequence to allow the control of transgene expression by the endogenous promoter of RAG1.

### Off-target Analysis

Preliminary in silico analysis demonstrated a promising off-target profile as shown by high MIT and CFD specificity scores (Table 1 and 2).

TABLE 1
Specificity analysis of exon 2 RAG1 gRNAs, including optional gRNAs
for exon 2 gene replacement strategy.

| | | | | | # OT | | | Out- | |
|---|---|---|---|---|---|---|---|---|---|
| | | | MIT | CFD | ≤4 | Doench | Moreno- | of- | |
| gRNA | Target | | Spec | Spec | mis- | ′16- | Mateos- | Frame- | Lindel- |
| ID | sequence | DSB site | Score | Score | matches | Score | Score | Score | Score |
| g1 M5 | TTGCTGGA | chr11: | 78 | 89 | 118 | 39 | 46 | 55 | 66 |
| ex2 | CATTTCAC | 36,574,368- | | | | | | | |
| RAG1 | CATCAGG | 36,574,369 | | | | | | | |
| g2 M5 | TGCTGGAC | chr11: | 73 | 86 | 184 | 54 | 52 | 53 | 62 |
| ex2 | ATTTCACCA | 36,574,367- | | | | | | | |
| RAG1 | TCAGGG | 36,574,368 | | | | | | | |
| g3 M5 | TCCAGCAA | chr11: | 73 | 81 | 195 | 76 | 39 | 63 | 84 |
| ex2 | AAGAGTGC | 36,574,394- | | | | | | | |
| RAG1 | AATGAGG | 36,574,395 | | | | | | | |
| g4 M4 | AAGCATGG | chr11: | 87 | 89 | 104 | 66 | 65 | 63 | 77 |
| ex2 | ATATCGGC | 36,574,294- | | | | | | | |
| RAG1 | AAGAGGG | 36,574,295 | | | | | | | |
| g5 M3 | AAGATGTA | chr11: | 75 | 87 | 136 | 54 | 34 | 53 | 71 |
| ex2 | TCTTACTG | 36,574,109- | | | | | | | |
| RAG1 | CAGTTGG | 36,574,110 | | | | | | | |
| g6 M2 | CGAGGAAC | chr11: | 75 | 86 | 93 | 64 | 58 | 66 | 78 |
| ex2 | GTGACCAT | 36,573,910- | | | | | | | |
| RAG1 | GGAGTGG | 36,573,911 | | | | | | | |
| g7 | GTTTAGCA | chr11: | 83 | 93 | 76 | 59 | 55 | 70 | 84 |
| exon2 | GTGCCCCA | 36,573,878- | | | | | | | |
| M2/3 | TGTGAGG | 36,573,879 | | | | | | | |
| g8 | CTTCCTCTT | chr11: | 87 | 89 | 110 | 55 | 76 | 71 | 72 |
| exon2 | GAGTCCCC | 36,573,959- | | | | | | | |
| M2/3 | GACGGG | 36,573,960 | | | | | | | |
| g9 | ATCTGCAA | chr11: | 95 | 95 | 47 | 61 | 66 | 69 | 74 |
| exon2 | CACTGCCC | 36,573,957- | | | | | | | |
| M2/3 | GTCGGGG | 36,573,958 | | | | | | | |
| g10 | TCGGGAAG | chr11: | 84 | 93 | 65 | 67 | 54 | 64 | 89 |
| exon2 | TAAACCTC | 36,573,879- | | | | | | | |
| M2/3 | ACATGGG | 36,573,880 | | | | | | | |
| g11 | CATGTGAG | chr11: | 89 | 91 | 80 | 66 | 50 | 70 | 84 |
| exon2 | GTTTACTTC | 36,573,892- | | | | | | | |
| M2/3 | CCGAGG | 36,573,893 | | | | | | | |
| g12 | ACATCTGC | chr11: | 92 | 94 | 66 | 65 | 42 | 63 | 83 |
| exon2 | AACACTGC | 36,573,955- | | | | | | | |
| M2/3 | CCGTCGG | 36,573,956 | | | | | | | |
| g13 | CGGGAAGT | chr11: | 79 | 93 | 77 | 71 | 63 | 69 | 79 |
| exon2 | AAACCTCA | 36,573,878- | | | | | | | |
| M2/3 | CATGGGG | 36,573,879 | | | | | | | |
| g14 | GTGCAATG | chr11: | 65 | 81 | 190 | 44 | 52 | 55 | 83 |
| exon2 | AGGAGGTC | 36,574,406- | | | | | | | |
| M5 | AGTTTGG | 36,574,407 | | | | | | | |
| g1 | AAGCCCTC | chr11: | 71 | 83 | 138 | 69 | 66 | 71 | 91 |
| 3′UTR | AATGCAAC | 36,576,484- | | | | | | | |
| | CCAGAGG | 36,576,485 | | | | | | | |
| g1 | GAGAGTCC | chr11: | 90 | 96 | 56 | 38 | 76 | 56 | 80 |
| ex2 | TCTATGCC | 36,576,390- | | | | | | | |
| | TAATGGG | 36,576,391 | | | | | | | |
| g2 | AGCCCTCA | chr11: | 78 | 87 | 120 | 66 | 46 | 70 | 76 |
| 3′UTR | ATGCAACC | 36,576,483- | | | | | | | |
| | CAGAGGG | 36,576,484 | | | | | | | |
| g2 | AGGGGACC | chr11: | 85 | 91 | 67 | 62 | 61 | 64 | 58 |
| ex2 | CATTAGGC | 36,576,395- | | | | | | | |
| | ATAGAGG | 36,576,396 | | | | | | | |
| g3 | TAGGGCAA | chr11: | 78 | 88 | 113 | 56 | 39 | 60 | 79 |
| 3′UTR | CCACTTAT | 36,576,454- | | | | | | | |
| | GAGTTGG | 36,576,455 | | | | | | | |
| g3 | AGAGAGTC | chr11: | 87 | 89 | 101 | 44 | 26 | 58 | 75 |
| ex2 | CTCTATGC | 36,576,391- | | | | | | | |
| | CTAATGG | 36,576,392 | | | | | | | |

TABLE 2
List of off-target sites with 1, 2 ore 3 mismatches for
each exon 2 RAG1 gRNA, including optional gRNAs
for exon 2 gene replacement strategy.

| | # | OT details |
|---|---|---|
| gRNA ID | mismatches | (intron/exon only) |
| g1 M5 ex2 RAG1 | 3 | exon:LNX1 |
| g1 M5 ex2 RAG1 | 3 | intron:APP |
| g1 M5 ex2 RAG1 | 2 | intron:SRBD1 |
| g2 M5 ex2 RAG1 | 3 | intron:CSNK1G3 |
| g2 M5 ex2 RAG1 | 3 | intron:SATB2 |
| g2 M5 ex2 RAG1 | 3 | exon:TRAF6 |
| g2 M5 ex2 RAG1 | 3 | intron:CRHR2 |
| g2 M5 ex2 RAG1 | 3 | intron:TEAD1 |
| g2 M5 ex2 RAG1 | 3 | intron:SRBD1 |
| g3 M5 ex2 RAG1 | 2 | exon:ITGA4 |
| g3 M5 ex2 RAG1 | 2 | intron:RP11-332J15.2 |
| g3 M5 ex2 RAG1 | 2 | exon:ZZEF1 |
| g4 M4 ex2 RAG1 | 3 | intron:HSD11B1 |
| g4 M4 ex2 RAG1 | 3 | exon:LRRN1 |
| g4 M4 ex2 RAG1 | 3 | exon:PRUNE |
| g5 M3 ex2 RAG1 | 3 | intron:NDRG1 |
| g5 M3 ex2 RAG1 | 3 | intron:RBFOX1 |
| g5 M3 ex2 RAG1 | 3 | intron:CUEDC1 |
| g5 M3 ex2 RAG1 | 3 | intron:LINC00371 |
| g5 M3 ex2 RAG1 | 2 | intron:FAM196A |
| g6 M2 ex2 RAG1 | 3 | intron:TSPAN18 |
| g6 M2 ex2 RAG1 | 3 | exon:PPAPDC3 |
| g6 M2 ex2 RAG1 | 3 | exon:KCNMA1 |
| g6 M2 ex2 RAG1 | 3 | exon:DLGAP4-AS1/DLGAP4 |
| g6 M2 ex2 RAG1 | 2 | intron:SLC25A13 |
| g7 exon2 M2/3 | 3 | intron:CCR2 |
| g7 exon2 M2/3 | 3 | intron:HMGXB3 |
| g8 exon2 M2/3 | 3 | intron:TRPM3 |
| g8 exon2 M2/3 | 3 | intron:RCAN2 |
| g8 exon2 M2/3 | 3 | exon:OPRD1/RP1-212P9.3 |
| g8 exon2 M2/3 | 2 | intron:PDLIM2/AC037459.4 |
| g9 exon2 M2/3 | 3 | intron:YBX3 |
| g9 exon2 M2/3 | 3 | intron:HAVCR1 |
| g9 exon2 M2/3 | 3 | intron:EFCAB5 |
| g9 exon2 M2/3 | 3 | exon:TLE2 |
| g10 exon2 M2/3 | 3 | intron:DTNA |
| g10 exon2 M2/3 | 3 | exon:PTDSS1 |
| g10 exon2 M2/3 | 3 | intron:TMEM245 |
| g11 exon2 M2/3 | 3 | intron:KCNQ1 |
| g11 exon2 M2/3 | 3 | intron:RP11-10O17.3 |
| g12 exon2 M2/3 | 3 | intron:SOS1 |
| g12 exon2 M2/3 | 3 | intron:RP11-73M18.2/KLC1 |
| g12 exon2 M2/3 | 3 | intron:MCC |
| g13 exon2 M2/3 | 3 | intron: MID1 |
| g13 exon2 M2/3 | 3 | intron:LPL |
| g13 exon2 M2/3 | 3 | intron:ATXN2 |
| g13 exon2 M2/3 | 3 | exon:RNFT2 |
| g14 exon2 M5 | 3 | intron:TBC1D8 |
| g14 exon2 M5 | 3 | exon:MAGI1 |
| g14 exon2 M5 | 3 | intron:MYLK |
| g14 exon2 M5 | 3 | intron:PTK2B |
| g14 exon2 M5 | 3 | intron:GRM4 |
| g14 exon2 M5 | 3 | intron:DCC |
| g14 exon2 M5 | 3 | exon:GPR144 |
| g14 exon2 M5 | 3 | intron:PREP |
| g1 3′UTR | 3 | intron:PARP8 |
| g1 3′UTR | 3 | intron:QSOX2 |
| g1 3′UTR | 3 | intron:SDK1 |
| g1 3′UTR | 3 | intron:FAM83F |
| g1 3′UTR | 3 | intron:FAM19A5 |
| g1 3′UTR | 3 | intron:KSR2 |
| g1 ex2 | 3 | intron:RP11-382E9.1 |
| g1 ex3 | 3 | intron: EBF2 |
| g2 3′UTR | 3 | intron:ALG6 |
| g2 3′UTR | 3 | intron:WWC3-AS1 |
| g2 3′UTR | 3 | intron:CFH |
| g2 3′UTR | 3 | intron:CTC-254B4.1 |
| g2 3′UTR | 3 | exon:AC006548.28/GAB4 |
| g2 3′UTR | 3 | intron:RAP1GAP |
| g2 3′UTR | 3 | intron:SHF |
| g2 3′UTR | 3 | intron:ABCG8 |
| g2 3′UTR | 2 | intron:KSR2 |
| g2 ex2 | 3 | intron:SDK1 |
| g2 ex3 | 3 | intron:SKAP2 |
| g2 ex4 | 2 | intron:CEP41 |
| g3 3′UTR | 3 | intron:STK24 |
| g3 3′UTR | 3 | intron:RP11-13N12.2 |
| g3 3′UTR | 3 | intron:NTNG1 |
| g3 3′UTR | 3 | intron: MYRIP |
| g3 3′UTR | 3 | intron:EXOC6B |
| g3 3′UTR | 3 | intron:SMS |
| g3 3′UTR | 3 | intron:RP11-629N8.3 |
| g3 ex2 | 3 | intron:MYO5A |
| g3 ex3 | 3 | exon:NUP205 |
| g3 ex4 | 3 | intron:RP11-382E9.1 |
| g3 ex5 | 3 | intron:ARR3 |

### Donor DNA Desian

Corrective donors carrying a coRAG1 partial CDS in frame with the upstream portion of the endogenous RAG1 were designed and synthesized. The partial CDS is flanked by the left and right homology arms designed according to each gRNA specificity. According to preliminary data on the guide selection, we designed three corrective donors for g5 and g6 gRNAs: one donor, carrying a short homology arm, will be tested for the “exon 2 RAG1 gene targeting” and two donors for the “exon 2 RAG1 gene replacement” strategy will exploit long right homology arms (1800 or 900 bp) to favor the HDR and gene replacement (FIG. 5A). Additional donors with a long right homology arm were designed and synthesized with homology arms specific for all other gRNAs (FIG. 5B). In parallel, we designed the corrective donor suitable for the “intron 1 RAG1 gene replacement” strategy (FIG. 5C).

### Material and Methods

gRNA and RNP Assembly

Cas9 protein and custom gRNAs were purchased from Integrated DNA Technologies (IDT) and assembled following the manufacturer protocol. Briefly, crRNA and trRNA were annealed heating them at 95° C. for 5 minutes and letting them slowly cool down at RT for 10 minutes. Cas9 protein was then incubated for 15 minutes at room temperature with the annealed guide RNA fragments, to assemble the ribonucleoprotein (RNP). Alternatively, some gRNAs were purchased from Synthego as a full length sgRNA and then assembled with Cas9 protein to generate the RNP.

Guide sequences are shown below (PAM sequences are highlighted in bold):

g1 M5 ex2 RAG1:
TTGCTGGACATTTCACCATC AGG
g2 M5 ex2 RAG1:
TGCTGGACATTTCACCATCA GGG
g3 M5 ex2 RAG1:
TCCAGCAAAAGAGTGCAATG AGG
g4 M4 ex2 RAG1:
AAGCATGGATATCGGCAAGA GGG
g5 M3 ex2 RAG1:
AAGATGTATCTTACTGCAGT TGG
g6 M2 ex2 RAG1:
CGAGGAACGTGACCATGGAG TGG
g7 exon2 M2/3:
GTTTAGCAGTGCCCCATGTG AGG
g8 exon2 M2/3:
CTTCCTCTTGAGTCCCCGAC GGG
g9 exon2 M2/3:
ATCTGCAACACTGCCCGTCG GGG
g10 exon2 M2/3:
TCGGGAAGTAAACCTCACAT GGG
g11 exon2 M2/3:
CATGTGAGGTTTACTTCCCG AGG
g12 exon2 M2/3:
ACATCTGCAACACTGCCCGT CGG
g13 exon2 M2/3:
CGGGAAGTAAACCTCACATG GGG
g14 exon2 M5:
GTGCAATGAGGAGGTCAGTT TGG
g1 ex2:
GAGAGTCCTCTATGCCTAAT GGG
g2 ex2:
AGGGGACCCATTAGGCATAG AGG
g3 ex2:
AGAGAGTCCTCTATGCCTAA TGG
g1 3′UTR:
AAGCCCTCAATGCAACCCAG AGG
g2 3′UTR:
AGCCCTCAATGCAACCCAGA GGG
g3 3′UTR:
TAGGGCAACCACTTATGAGT TGG
g14 for KO:
AACATCTTCTGTCGCTGACT CGG
g9:
GTCAGATGGCAATGTCGAGA TGG

### NHEJ Efficiency

Indels induced by NHEJ were measured by a mismatch selective endonuclease assay using the T7 endonuclease (T7E1). Briefly, gDNA of gene edited cells was extracted and amplified by PCR with primers flanking the Cas9 RNP target site. The PCR product was denatured, slowly re-annealed and digested with T7 endonuclease (New England BioLabs) for 1 h, 37°. T7 nuclease only cut DNA at sites where there is a mismatch between the DNA strands, thus between re-annealed wild type and mutant alleles. Fragments were separated on 4200 Tape Station System (Agilent) and analyzed by the provided software. The ratio of the uncleaved parental fragment versus cleaved fragments was calculated and it gives a good estimation of NHEJ efficiency of the artificial nuclease. Calculation of % NHEJ: (sum cleaved fragment)/(sum cleaved fragments+parental fragment)×100. Alternatively, we measured indels induced by NHEJ by TIDE analysis of Sanger sequences (tracking of indels by decomposition; (http://shinyapps.datacurators.nl/tide/).

Primers used for NHEJ assay are shown below according to the gRNA specificity:
Primers for g1 M5 ex2 RAG1, g2 M5 ex2 RAG1, g3 M5 ex2 RAG1, g4 M4 ex2 RAG1, g5 M3 ex2 RAG1, g6 M2 ex2 RAG1 gRNAs (Exonic strategy):

FW:
AAGAGAGCTACTTCCTGGCC
RV:
GCACACGGACTTCACATCTC

Primers for g7 exon2 M2/3, g10 exon2 M2/3, g11 exon2 M2/3, g13 exon2 M2 gRNAs (Exonic strategy):

FW:
AGCCAACCTTCGACATCTCT
RV:
CAAAGTGCTCTGGGAAGTCC

Primers for g8 exon2 M2/3, g9 exon2 M2/3, g12 exon2 M2/3 gRNAs (Exonic strategy):

FW:
AGTGCCCCATGTGAGGTTTA
RV:
CATCAGGGAATTCAAGACGCT

Primers for g14 exon2 M5 gRNA (Exonic strategy):

FW:
AGGATCAGCAGCAAGGATGT
RV:
GCACACGGACTTCACATCTC

Primers for g1 ex2, g2 ex2, g3 ex2, g1 3′UTR, g2 3′UTR, g2 3′UTR gRNAs (optional gRNAs for replacement strategies):

FW:
GCTGAGCTCCTTTCTACGAAGT
RV:
GAAAACCACAAGACCAATTTCTTTC

Primers for g14 for KO gRNA:

FW:
TCCATGCTTCCCTACTGAC
RV:
CTCCCATTCCATCACAAGAC

Primers for g9 gRNA (Intronic strategy):

FW:
GAAGTGGTTCATGCAAGAGG
RV:
GGATGAACATGGAGAAAGCAG

### Off-Target Analysis

In silico prediction of off-target profile was performed with CRISPOR (http://crispor.tefor.net) to search genomes for potential CRISPR off-target sites.

gRNA Delivering in Cell Lines and CD34+ Cells

A dose of 2×10⁵/5×10⁵ NALM6 or K562 cells per well were electroporated with RNPs selecting the specific nucleofector program (Lonza, SF Cell line). For gRNA delivering in HSPC, CD34+ cells derived from mPB of HD were thawed at day 0 and prestimulated for three days seeding 0.5×10⁶ cells/ml in StemSpan medium supplemented with penicillin/streptomycin antibiotics and early-acting cytokines: Stem cell factor (SCF) 300 ng/ml, Flt3 ligand (Flt3-L) 300 ng/ml, Thrombopoietin (TPO) 100 ng/ml, StemRegenin1 (SR1) (1 uM), UM171 35 nM and 16,16-dimethyl prostaglandin E2 (dmPGE2) (10 uM). At day 3, gRNAs were delivered as an in vitro preassembled RNPs (25-50 pmol/well) by electroporation. After the gRNA delivering, cells were kept in culture and used or stored for molecular and phenotypic analyses.

### Donor Constructs

We designed the donor constructs according to the gene editing strategies and gRNA specificities. Donor templates have been synthetized and cloned by gene synthesis services (GenScript).

Sequences of vector inserts with main features are reported below:

DONOR specific for “g5 M3 ex2 RAG1”
gRNA for the exon 2 RAG1 gene targeting
strategy
INSERT
gatccatcaagccaaccttcgacatctctgccgcatctgtgggaa
ttcttttagagctgatgagcacaacaggagatatccagtccatgg
tcctgtggatggtaaaaccctaggccttttacgaaagaaggaaaa
gagagctacttcctggccggacctcattgccaaggttttccggat
cgatgtgaaggcagatgttgactcgatccaccccactgagttctg
ccataactgctggagcatcatgcacaggaagtttagcagtgcccc
atgtgaggtttacttcccgaggaacgtgaccatggagtggcaccc
ccacacaccatcctgtgacatctgcaacactgcccgtcggggact
caagaggaagagtcttcagccaaacttgcagctcagcaaaaaact
caaaactgtgcttgaccaagcaagacaagcccgtcagcgcaagag
aagagctcaggcaaggatcagcagcaaggatgtcatgaagaagat
cgcaaactgcagcaagatccacctgagcaccaaactgctggccgt
ggacttccctgagcacttcgtgaagtccatcagctgccagatctg
cgagcacatcctggccgatcctgtggaaacaaactgcaagcacgt
gttctgcagagtgtgcatcctgcggtgcctgaaagtgatgggcag
ctactgcccctcctgcagatacccttgcttccccaccgatctgga
aagccctgtgaagtccttcctgagcgtgctgaacagcctgatggt
caagtgccccgccaaagaatgcaacgaggaagtgtccctggaaaa
gtacaaccaccacatcagcagccacaaagagtccaaagaaatctt
cgtgcacatcaacaaaggcggcagaccccggcagcatctgctgtc
tcttacaagacgggcccagaagcaccggctgagagaactgaagct
gcaagtgaaggcctttgccgacaaagaggaaggcggcgacgtcaa
gagcgtgtgcatgaccctgtttctgctggccctgagagcccggaa
tgagcatagacaggccgatgagctggaagccatcatgcaaggcaa
aggcagcggactgcagcctgctgtgtgtctggctatcagagtgaa
caccttcctgtcctgcagccagtaccacaagatgtaccggaccgt
gaaggccattaccggcagacagatcttccagcctctgcacgccct
gagaaacgccgagaaagttctgctgcctggctaccaccacttcga
gtggcagcctccactgaagaacgtgtccagcagcaccgacgtggg
catcatcgatggactgagcggactgtctagcagcgtggacgacta
ccccgtggacacaatcgccaagcggttcagatacgacagcgccct
ggtgtctgccctgatggacatggaagaggacatcctggaaggcat
gcggagccaggacctggacgattacctgaacggccctttcaccgt
ggtggtcaaagaaagctgtgacggcatgggcgacgtgtccgagaa
acacggatctggacctgtggtgccagagaaggccgtgcggttcag
cttcaccatcatgaagatcactatcgcccacagcagccagaacgt
gaaagtgttcgaggaagccaagcctaacagcgagctgtgctgcaa
gcctctgtgtctgatgctggccgacgagagcgatcacgagacact
gaccgccattctgagccctctgatcgccgaacgggaagccatgaa
gtcctccgagctgatgctcgaactcggcggcatcctgagaacctt
caagttcatcttccgcggcaccggctacgacgagaagctcgttag
agaggtggaaggcctggaagcctctggcagcgtgtacatctgcac
cctgtgtgacgccaccagactggaagctagccagaacctggtgtt
ccacagcatcaccagaagccacgccgaaaacctggaaagatacga
agtgtggcggagcaacccctaccacgagagcgtggaagaactgcg
ggatagagtgaagggcgtgtccgccaagcctttcatcgagacagt
gcctagcatcgacgccctgcactgcgatattggcaacgccgccga
attctacaagatctttcagctggaaatcggcgaggtgtacaagaa
ccccaacgcctctaaagaggaacggaagcgctggcaggccacact
ggataagcacctgagaaagaagatgaatctgaagcccatcatgag
gatgaacggcaacttcgcccggaagctgatgaccaaagaaaccgt
ggatgccgtgtgcgagctgatcccctctgaggaaagacacgaggc
cctgcgggaactgatggacctgtacctgaagatgaagcccgtgtg
gcggtctagctgtcctgccaaagagtgccctgagtctctgtgcca
gtacagcttcaacagccagagattcgccgagctgctgtccaccaa
gttcaagtacagatacgagggcaagatcaccaactacttccacaa
gaccctggctcacgtgcccgagatcatcgagagagatggctctat
tggcgcctgggcctctgagggcaatgagtctggcaacaagctgtt
ccggcggttccgcaagatgaacgccagacagagcaagtgctacga
gatggaagatgtgctgaagcaccactggctgtacaccagcaagta
cctgcagaaattcatgaacgcccacaacgccctcaagaccagcgg
ctttaccatgaatcctcaggccagcctgggcgaccctctgggaat
tgaggatagcctggaatcccaggacagcatggaattctgataagc
agtaagatacatcttagtaccaagctccttgcagtggacttccca
gagcactttgtgaaatccatctcctgccagatctgtgaacacatt
ctggctgaccctgtggagaccaactgtaagcatgtcttttgccgg
gtctgcattctcagatgcctcaaagtcatgggcagctattgtccc
tcttgccgatatccatgcttccctactgacctggagagtccagtg
aagtcctttctg agcgtcttgaa
Left HA
gatccatcaagccaaccttcgacatctctgccgcatctgtgggaa
ttcttttagagctgatgagcacaacaggagatatccagtccatgg
tcctgtggatggtaaaaccctaggccttttacgaaagaaggaaaa
gagagctacttcctggccggacctcattgccaaggttttccggat
cgatgtgaaggcagatgttgactcgatccaccccactgagttctg
ccataactgctggagcatcatgcacaggaagtttagcagtgcccc
atgtgaggtttacttcccgaggaacgtgaccatggagtggcaccc
ccacacaccatcctgtgacatctgcaacactgcccgtcggggact
caagaggaagagtcttcagccaaacttgcagctcagcaaaaaact
caaaactgtgcttgaccaagcaagacaagcccgtcagcgcaagag
aagagctcaggcaaggatcagcagcaaggatgtcatgaagaagat
cgcaaact
coRAG1 CDS
gcagcaagatccacctgagcaccaaactgctggccgtggacttcc
ctgagcacttcgtgaagtccatcagctgccagatctgcgagcaca
tcctggccgatcctgtggaaacaaactgcaagcacgtgttctgca
gagtgtgcatcctgcggtgcctgaaagtgatgggcagctactgcc
cctcctgcagatacccttgcttccccaccgatctggaaagccctg
tgaagtccttcctgagcgtgctgaacagcctgatggtcaagtgcc
ccgccaaagaatgcaacgaggaagtgtccctggaaaagtacaacc
accacatcagcagccacaaagagtccaaagaaatcttcgtgcaca
tcaacaaaggcggcagaccccggcagcatctgctgtctcttacaa
gacgggcccagaagcaccggctgagagaactgaagctgcaagtga
aggcctttgccgacaaagaggaaggcggcgacgtcaagagcgtgt
gcatgaccctgtttctgctggccctgagagcccggaatgagcata
gacaggccgatgagctggaagccatcatgcaaggcaaaggcagcg
gactgcagcctgctgtgtgtctggctatcagagtgaacaccttcc
tgtcctgcagccagtaccacaagatgtaccggaccgtgaaggcca
ttaccggcagacagatcttccagcctctgcacgccctgagaaacg
ccgagaaagttctgctgcctggctaccaccacttcgagtggcagc
ctccactgaagaacgtgtccagcagcaccgacgtgggcatcatcg
atggactgagcggactgtctagcagcgtggacgactaccccgtgg
acacaatcgccaagcggttcagatacgacagcgccctggtgtctg
ccctgatggacatggaagaggacatcctggaaggcatgcggagcc
aggacctggacgattacctgaacggccctttcaccgtggtggtca
aagaaagctgtgacggcatgggcgacgtgtccgagaaacacggat
ctggacctgtggtgccagagaaggccgtgcggttcagcttcacca
tcatgaagatcactatcgcccacagcagccagaacgtgaaagtgt
tcgaggaagccaagcctaacagcgagctgtgctgcaagcctctgt
gtctgatgctggccgacgagagcgatcacgagacactgaccgcca
ttctgagccctctgatcgccgaacgggaagccatgaagtcctccg
agctgatgctcgaactcggcggcatcctgagaaccttcaagttca
tcttccgcggcaccggctacgacgagaagctcgttagagaggtgg
aaggcctggaagcctctggcagcgtgtacatctgcaccctgtgtg
acgccaccagactggaagctagccagaacctggtgttccacagca
tcaccagaagccacgccgaaaacctggaaagatacgaagtgtggc
ggagcaacccctaccacgagagcgtggaagaactgcgggatagag
tgaagggcgtgtccgccaagcctttcatcgagacagtgcctagca
tcgacgccctgcactgcgatattggcaacgccgccgaattctaca
agatctttcagctggaaatcggcgaggtgtacaagaaccccaacg
cctctaaagaggaacggaagcgctggcaggccacactggataagc
acctgagaaagaagatgaatctgaagcccatcatgaggatgaacg
gcaacttcgcccggaagctgatgaccaaagaaaccgtggatgccg
tgtgcgagctgatcccctctgaggaaagacacgaggccctgcggg
aactgatggacctgtacctgaagatgaagcccgtgtggcggtcta
gctgtcctgccaaagagtgccctgagtctctgtgccagtacagct
tcaacagccagagattcgccgagctgctgtccaccaagttcaagt
acagatacgagggcaagatcaccaactacttccacaagaccctgg
ctcacgtgcccgagatcatcgagagagatggctctattggcgcct
gggcctctgagggcaatgagtctggcaacaagctgttccggcggt
tccgcaagatgaacgccagacagagcaagtgctacgagatggaag
atgtgctgaagcaccactggctgtacaccagcaagtacctgcaga
aattcatgaacgcccacaacgccctcaagaccagcggctttacca
tgaatcctcaggccagcctgggcgaccctctgggaattgaggata
gcctggaatcccaggacagcatggaattctga
Right HA
gcagtaagatacatcttagtaccaagctccttgcagtggacttcc
cagagcactttgtgaaatccatctcctgccagatctgtgaacaca
ttctggctgaccctgtggagaccaactgtaagcatgtcttttgcc
gggtctgcattctcagatgcctcaaagtcatgggcagctattgtc
cctcttgccgatatccatgcttccctactgacctggagagtccag
tgaagtcctttctgagcgtcttgaa
DONOR specific for “g5 M3 ex2 RAG1”
gRNA for the exon 2 RAG1 gene replacement
strategy with long right HA
INSERT
aaaaccctaggccttttacgaaagaaggaaaagagagctacttcc
tggccggacctcattgccaaggttttccggatcgatgtgaaggca
gatgttgactcgatccaccccactgagttctgccataactgctgg
agcatcatgcacaggaagtttagcagtgccccatgtgaggtttac
ttcccgaggaacgtgaccatggagtggcacccccacacaccatcc
tgtgacatctgcaacactgcccgtcggggactcaagaggaagagt
cttcagccaaacttgcagctcagcaaaaaactcaaaactgtgctt
gaccaagcaagacaagcccgtcagcgcaagagaagagctcaggca
aggatcagcagcaaggatgtcatgaagaagatcgcaaactgcagc
aagatccacctgagcaccaaactgctggccgtggacttccctgag
cacttcgtgaagtccatcagctgccagatctgcgagcacatcctg
gccgatcctgtggaaacaaactgcaagcacgtgttctgcagagtg
tgcatcctgcggtgcctgaaagtgatgggcagctactgcccctcc
tgcagatacccttgcttccccaccgatctggaaagccctgtgaag
tccttcctgagcgtgctgaacagcctgatggtcaagtgccccgcc
aaagaatgcaacgaggaagtgtccctggaaaagtacaaccaccac
atcagcagccacaaagagtccaaagaaatcttcgtgcacatcaac
aaaggcggcagaccccggcagcatctgctgtctcttacaagacgg
gcccagaagcaccggctgagagaactgaagctgcaagtgaaggcc
tttgccgacaaagaggaaggcggcgacgtcaagagcgtgtgcatg
accctgtttctgctggccctgagagcccggaatgagcatagacag
gccgatgagctggaagccatcatgcaaggcaaaggcagcggactg
cagcctgctgtgtgtctggctatcagagtgaacaccttcctgtcc
tgcagccagtaccacaagatgtaccggaccgtgaaggccattacc
ggcagacagatcttccagcctctgcacgccctgagaaacgccgag
aaagttctgctgcctggctaccaccacttcgagtggcagcctcca
ctgaagaacgtgtccagcagcaccgacgtgggcatcatcgatgga
ctgagcggactgtctagcagcgtggacgactaccccgtggacaca
atcgccaagcggttcagatacgacagcgccctggtgtctgccctg
atggacatggaagaggacatcctggaaggcatgcggagccaggac
ctggacgattacctgaacggccctttcaccgtggtggtcaaagaa
agctgtgacggcatgggcgacgtgtccgagaaacacggatctgga
cctgtggtgccagagaaggccgtgcggttcagcttcaccatcatg
aagatcactatcgcccacagcagccagaacgtgaaagtgttcgag
gaagccaagcctaacagcgagctgtgctgcaagcctctgtgtctg
atgctggccgacgagagcgatcacgagacactgaccgccattctg
agccctctgatcgccgaacgggaagccatgaagtcctccgagctg
atgctcgaactcggcggcatcctgagaaccttcaagttcatcttc
cgcggcaccggctacgacgagaagctcgttagagaggtggaaggc
ctggaagcctctggcagcgtgtacatctgcaccctgtgtgacgcc
accagactggaagctagccagaacctggtgttccacagcatcacc
agaagccacgccgaaaacctggaaagatacgaagtgtggcggagc
aacccctaccacgagagcgtggaagaactgcgggatagagtgaag
ggcgtgtccgccaagcctttcatcgagacagtgcctagcatcgac
gccctgcactgcgatattggcaacgccgccgaattctacaagatc
tttcagctggaaatcggcgaggtgtacaagaaccccaacgcctct
aaagaggaacggaagcgctggcaggccacactggataagcacctg
agaaagaagatgaatctgaagcccatcatgaggatgaacggcaac
ttcgcccggaagctgatgaccaaagaaaccgtggatgccgtgtgc
gagctgatcccctctgaggaaagacacgaggccctgcgggaactg
atggacctgtacctgaagatgaagcccgtgtggcggtctagctgt
cctgccaaagagtgccctgagtctctgtgccagtacagcttcaac
agccagagattcgccgagctgctgtccaccaagttcaagtacaga
tacgagggcaagatcaccaactacttccacaagaccctggctcac
gtgcccgagatcatcgagagagatggctctattggcgcctgggcc
tctgagggcaatgagtctggcaacaagctgttccggcggttccgc
aagatgaacgccagacagagcaagtgctacgagatggaagatgtg
ctgaagcaccactggctgtacaccagcaagtacctgcagaaattc
atgaacgcccacaacgccctcaagaccagcggctttaccatgaat
cctcaggccagcctgggcgatcctttaggcatagaggactctctg
gaaagccaagattcaatggaattttaagtagggcaaccacttatg
agttggtttttgcaattgagtttccctctgggttgcattgagggc
ttctcctagcaccctttactgctgtgtatggggcttcaccatcca
agaggtggtaggttggagtaagatgctacagatgctctcaagtca
ggaatagaaactgatgagctgattgcttgaggcttttagtgagtt
ccgaaaagcaacaggaaaaatcagttatctgaaagctcagtaact
cagaacaggagtaactgcaggggaccagagatgagcaaagatctg
tgtgtgttggggagctgtcatgtaaatcaaagccaaggttgtcaa
agaacagccagtgaggccaggaaagaaattggtcttgtggttttc
atttttttcccccttgattgattatattttgtattgagatatgat
aagtgccttctatttcatttttgaataattcttcatttttataat
tttacatatcttggcttgctatataagattcaaaagagcttttta
aatttttctaataatatcttacatttgtacagcatgatgaccttt
acaaagtgctctcaatgcatttacccattcgttatataaatatgt
tacatcaggacaactttgagaaaatcagtccttttttatgtttaa
attatgtatctattgtaaccttcagagtttaggaggtcatctgct
gtcatggatttttcaataatgaatttagaatacacctgttagcta
cagttagttattaaatcttctgataatatatgtttacttagctat
cagaagccaagtatgattctttatttttactttttcatttcaaga
aatttagagtttccaaatttagagcttctgcatacagtcttaaag
ccacagaggcttgtaaaaatataggttagcttgatgtctaaaaat
atatttcatgtcttactgaaacattttgccagactttctccaaat
gaaacctgaatcaatttttctaaatctaggtttcatagagtcctc
tcctctgcaatgtgttattctttctataatgatcagtttactttc
agtggattcagaattgtgtagcaggataaccttgtatttttccat
ccgctaagtttagatggagtccaaacgcagtacagcagaagagtt
aacatttacacagtgctttttaccactgtggaatgttttcacact
catttttccttacaacaattctgaggagtaggtgttgttattatc
tccatttgatgggggtttaaatgatttgctcaaagtcatttaggg
gtaataaatacttggcttggaaatttaacacagtccttttgtctc
caaagcccttcttctttccaccacaaattaatcactatgtttata
aggtagtatcagaatttttttaggattcacaactaatcactatag
cacatgaccttgggattacatttttatggggcaggggtaagcaag
tttttaaatcatttgtgtgctctggctcttttgatagaagaaagc
aacacaaaagctccaaagggccccctaaccctcttgtggctccag
ttatttggaaactatgatctgcatccttaggaatctgggatttgc
cagttgctggcaatgtagagcaggcatggaattttatatgctagt
gagtcataatgatatgttagtgttaattagttttttcttcctttg
attttattggccataattgctactcttcatacacagtatatcaaa
gagcttgataatttagttgtcaaaag
Left HA
aaaaccctaggccttttacgaaagaaggaaaagagagctacttcc
tggccggacctcattgccaaggttttccggatcgatgtgaaggca
gatgttgactcgatccaccccactgagttctgccataactgctgg
agcatcatgcacaggaagtttagcagtgccccatgtgaggtttac
ttcccgaggaacgtgaccatggagtggcacccccacacaccatcc
tgtgacatctgcaacactgcccgtcggggactcaagaggaagagt
cttcagccaaacttgcagctcagcaaaaaactcaaaactgtgctt
gaccaagcaagacaagcccgtcagcgcaagagaagagctcaggca
aggatcagcagcaaggatgtcatgaagaagatcgcaaact
coRAG1 CDS
gcagcaagatccacctgagcaccaaactgctggccgtggacttcc
ctgagcacttcgtgaagtccatcagctgccagatctgcgagcaca
tcctggccgatcctgtggaaacaaactgcaagcacgtgttctgca
gagtgtgcatcctgcggtgcctgaaagtgatgggcagctactgcc
cctcctgcagatacccttgcttccccaccgatctggaaagccctg
tgaagtccttcctgagcgtgctgaacagcctgatggtcaagtgcc
ccgccaaagaatgcaacgaggaagtgtccctggaaaagtacaacc
accacatcagcagccacaaagagtccaaagaaatcttcgtgcaca
tcaacaaaggcggcagaccccggcagcatctgctgtctcttacaa
gacgggcccagaagcaccggctgagagaactgaagctgcaagtga
aggcctttgccgacaaagaggaaggcggcgacgtcaagagcgtgt
gcatgaccctgtttctgctggccctgagagcccggaatgagcata
gacaggccgatgagctggaagccatcatgcaaggcaaaggcagcg
gactgcagcctgctgtgtgtctggctatcagagtgaacaccttcc
tgtcctgcagccagtaccacaagatgtaccggaccgtgaaggcca
ttaccggcagacagatcttccagcctctgcacgccctgagaaacg
ccgagaaagttctgctgcctggctaccaccacttcgagtggcagc
ctccactgaagaacgtgtccagcagcaccgacgtgggcatcatcg
atggactgagcggactgtctagcagcgtggacgactaccccgtgg
acacaatcgccaagcggttcagatacgacagcgccctggtgtctg
ccctgatggacatggaagaggacatcctggaaggcatgcggagcc
aggacctggacgattacctgaacggccctttcaccgtggtggtca
aagaaagctgtgacggcatgggcgacgtgtccgagaaacacggat
ctggacctgtggtgccagagaaggccgtgcggttcagcttcacca
tcatgaagatcactatcgcccacagcagccagaacgtgaaagtgt
tcgaggaagccaagcctaacagcgagctgtgctgcaagcctctgt
gtctgatgctggccgacgagagcgatcacgagacactgaccgcca
ttctgagccctctgatcgccgaacgggaagccatgaagtcctccg
agctgatgctcgaactcggcggcatcctgagaaccttcaagttca
tcttccgcggcaccggctacgacgagaagctcgttagagaggtgg
aaggcctggaagcctctggcagcgtgtacatctgcaccctgtgtg
acgccaccagactggaagctagccagaacctggtgttccacagca
tcaccagaagccacgccgaaaacctggaaagatacgaagtgtggc
ggagcaacccctaccacgagagcgtggaagaactgcgggatagag
tgaagggcgtgtccgccaagcctttcatcgagacagtgcctagca
tcgacgccctgcactgcgatattggcaacgccgccgaattctaca
agatctttcagctggaaatcggcgaggtgtacaagaaccccaacg
cctctaaagaggaacggaagcgctggcaggccacactggataagc
acctgagaaagaagatgaatctgaagcccatcatgaggatgaacg
gcaacttcgcccggaagctgatgaccaaagaaaccgtggatgccg
tgtgcgagctgatcccctctgaggaaagacacgaggccctgcggg
aactgatggacctgtacctgaagatgaagcccgtgtggcggtcta
gctgtcctgccaaagagtgccctgagtctctgtgccagtacagct
tcaacagccagagattcgccgagctgctgtccaccaagttcaagt
acagatacgagggcaagatcaccaactacttccacaagaccctgg
ctcacgtgcccgagatcatcgagagagatggctctattggcgcct
gggcctctgagggcaatgagtctggcaacaagctgttccggcggt
tccgcaagatgaacgccagacagagcaagtgctacgagatggaag
atgtgctgaagcaccactggctgtacaccagcaagtacctgcaga
aattcatgaacgcccacaacgccctcaagaccagcggctttacca
tgaatcctcaggccagcctgggcgatccttt
Right HA
aggcatagaggactctctggaaagccaagattcaatggaatttta
agtagggcaaccacttatgagttggtttttgcaattgagtttccc
tctgggttgcattgagggcttctcctagcaccctttactgctgtg
tatggggcttcaccatccaagaggtggtaggttggagtaagatgc
tacagatgctctcaagtcaggaatagaaactgatgagctgattgc
ttgaggcttttagtgagttccgaaaagcaacaggaaaaatcagtt
atctgaaagctcagtaactcagaacaggagtaactgcaggggacc
agagatgagcaaagatctgtgtgtgttggggagctgtcatgtaaa
tcaaagccaaggttgtcaaagaacagccagtgaggccaggaaaga
aattggtcttgtggttttcatttttttcccccttgattgattata
ttttgtattgagatatgataagtgccttctatttcatttttgaat
aattcttcatttttataattttacatatcttggcttgctatataa
gattcaaaagagctttttaaatttttctaataatatcttacattt
gtacagcatgatgacctttacaaagtgctctcaatgcatttaccc
attcgttatataaatatgttacatcaggacaactttgagaaaatc
agtccttttttatgtttaaattatgtatctattgtaaccttcaga
gtttaggaggtcatctgctgtcatggatttttcaataatgaattt
agaatacacctgttagctacagttagttattaaatcttctgataa
tatatgtttacttagctatcagaagccaagtatgattctttattt
ttactttttcatttcaagaaatttagagtttccaaatttagagct
tctgcatacagtcttaaagccacagaggcttgtaaaaatataggt
tagcttgatgtctaaaaatatatttcatgtcttactgaaacattt
tgccagactttctccaaatgaaacctgaatcaatttttctaaatc
taggtttcatagagtcctctcctctgcaatgtgttattctttcta
taatgatcagtttactttcagtggattcagaattgtgtagcagga
taaccttgtatttttccatccgctaagtttagatggagtccaaac
gcagtacagcagaagagttaacatttacacagtgctttttaccac
tgtggaatgttttcacactcatttttccttacaacaattctgagg
agtaggtgttgttattatctccatttgatgggggtttaaatgatt
tgctcaaagtcatttaggggtaataaatacttggcttggaaattt
aacacagtccttttgtctccaaagcccttcttctttccaccacaa
attaatcactatgtttataaggtagtatcagaatttttttaggat
tcacaactaatcactatagcacatgaccttgggattacattttta
tggggcaggggtaagcaagtttttaaatcatttgtgtgctctggc
tcttttgatagaagaaagcaacacaaaagctccaaagggccccct
aaccctcttgtggctccagttatttggaaactatgatctgcatcc
ttaggaatctgggatttgccagttgctggcaatgtagagcaggca
tggaattttatatgctagtgagtcataatgatatgttagtgttaa
ttagttttttcttcctttgattttattggccataattgctactct
tcatacacagtatatcaaagagcttgataatttagttgtcaaaag
DONOR specific for “g5 M3 ex2 RAG1”
gRNA for the exon 2 RAG1 gene replacement
strategy with short right HA
INSERT
aaaaccctaggccttttacgaaagaaggaaaagagagctacttcc
tggccggacctcattgccaaggttttccggatcgatgtgaaggca
gatgttgactcgatccaccccactgagttctgccataactgctgg
agcatcatgcacaggaagtttagcagtgccccatgtgaggtttac
ttcccgaggaacgtgaccatggagtggcacccccacacaccatcc
tgtgacatctgcaacactgcccgtcggggactcaagaggaagagt
cttcagccaaacttgcagctcagcaaaaaactcaaaactgtgctt
gaccaagcaagacaagcccgtcagcgcaagagaagagctcaggca
aggatcagcagcaaggatgtcatgaagaagatcgcaaactgcagc
aagatccacctgagcaccaaactgctggccgtggacttccctgag
cacttcgtgaagtccatcagctgccagatctgcgagcacatcctg
gccgatcctgtggaaacaaactgcaagcacgtgttctgcagagtg
tgcatcctgcggtgcctgaaagtgatgggcagctactgcccctcc
tgcagatacccttgcttccccaccgatctggaaagccctgtgaag
tccttcctgagcgtgctgaacagcctgatggtcaagtgccccgcc
aaagaatgcaacgaggaagtgtccctggaaaagtacaaccaccac
atcagcagccacaaagagtccaaagaaatcttcgtgcacatcaac
aaaggcggcagaccccggcagcatctgctgtctcttacaagacgg
gcccagaagcaccggctgagagaactgaagctgcaagtgaaggcc
tttgccgacaaagaggaaggcggcgacgtcaagagcgtgtgcatg
accctgtttctgctggccctgagagcccggaatgagcatagacag
gccgatgagctggaagccatcatgcaaggcaaaggcagcggactg
cagcctgctgtgtgtctggctatcagagtgaacaccttcctgtcc
tgcagccagtaccacaagatgtaccggaccgtgaaggccattacc
ggcagacagatcttccagcctctgcacgccctgagaaacgccgag
aaagttctgctgcctggctaccaccacttcgagtggcagcctcca
ctgaagaacgtgtccagcagcaccgacgtgggcatcatcgatgga
ctgagcggactgtctagcagcgtggacgactaccccgtggacaca
atcgccaagcggttcagatacgacagcgccctggtgtctgccctg
atggacatggaagaggacatcctggaaggcatgcggagccaggac
ctggacgattacctgaacggccctttcaccgtggtggtcaaagaa
agctgtgacggcatgggcgacgtgtccgagaaacacggatctgga
cctgtggtgccagagaaggccgtgcggttcagcttcaccatcatg
aagatcactatcgcccacagcagccagaacgtgaaagtgttcgag
gaagccaagcctaacagcgagctgtgctgcaagcctctgtgtctg
atgctggccgacgagagcgatcacgagacactgaccgccattctg
agccctctgatcgccgaacgggaagccatgaagtcctccgagctg
atgctcgaactcggcggcatcctgagaaccttcaagttcatcttc
cgcggcaccggctacgacgagaagctcgttagagaggtggaaggc
ctggaagcctctggcagcgtgtacatctgcaccctgtgtgacgcc
accagactggaagctagccagaacctggtgttccacagcatcacc
agaagccacgccgaaaacctggaaagatacgaagtgtggcggagc
aacccctaccacgagagcgtggaagaactgcgggatagagtgaag
ggcgtgtccgccaagcctttcatcgagacagtgcctagcatcgac
gccctgcactgcgatattggcaacgccgccgaattctacaagatc
tttcagctggaaatcggcgaggtgtacaagaaccccaacgcctct
aaagaggaacggaagcgctggcaggccacactggataagcacctg
agaaagaagatgaatctgaagcccatcatgaggatgaacggcaac
ttcgcccggaagctgatgaccaaagaaaccgtggatgccgtgtgc
gagctgatcccctctgaggaaagacacgaggccctgcgggaactg
atggacctgtacctgaagatgaagcccgtgtggcggtctagctgt
cctgccaaagagtgccctgagtctctgtgccagtacagcttcaac
agccagagattcgccgagctgctgtccaccaagttcaagtacaga
tacgagggcaagatcaccaactacttccacaagaccctggctcac
gtgcccgagatcatcgagagagatggctctattggcgcctgggcc
tctgagggcaatgagtctggcaacaagctgttccggcggttccgc
aagatgaacgccagacagagcaagtgctacgagatggaagatgtg
ctgaagcaccactggctgtacaccagcaagtacctgcagaaattc
atgaacgcccacaacgccctcaagaccagcggctttaccatgaat
cctcaggccagcctgggcgatcctttaggcatagaggactctctg
gaaagccaagattcaatggaattttaagtagggcaaccacttatg
agttggtttttgcaattgagtttccctctgggttgcattgagggc
ttctcctagcaccctttactgctgtgtatggggcttcaccatcca
agaggtggtaggttggagtaagatgctacagatgctctcaagtca
ggaatagaaactgatgagctgattgcttgaggcttttagtgagtt
ccgaaaagcaacaggaaaaatcagttatctgaaagctcagtaact
cagaacaggagtaactgcaggggaccagagatgagcaaagatctg
tgtgtgttggggagctgtcatgtaaatcaaagccaaggttgtcaa
agaacagccagtgaggccaggaaagaaattggtcttgtggttttc
atttttttcccccttgattgattatattttgtattgagatatgat
aagtgccttctatttcatttttgaataattcttcatttttataat
tttacatatcttggcttgctatataagattcaaaagagcttttta
aatttttctaataatatcttacatttgtacagcatgatgaccttt
acaaagtgctctcaatgcatttacccattcgttatataaatatgt
tacatcaggacaactttgagaaaatcagtccttttttatgtttaa
attatgtatctattgtaaccttcagagtttaggaggtcatctgct
gtcatggatttttcaataatgaatttagaatacacctgttagcta
cagttagttattaaatcttctgataatatatgtttacttagctat
cagaagccaagtatgattctttatttttactttttcatttcaaga
aatttagagtttccaaatttagagct
Left HA
aaaaccctaggccttttacgaaagaaggaaaagagagctacttcc
tggccggacctcattgccaaggttttccggatcgatgtgaaggca
gatgttgactcgatccaccccactgagttctgccataactgctgg
agcatcatgcacaggaagtttagcagtgccccatgtgaggtttac
ttcccgaggaacgtgaccatggagtggcacccccacacaccatcc
tgtgacatctgcaacactgcccgtcggggactcaagaggaagagt
cttcagccaaacttgcagctcagcaaaaaactcaaaactgtgctt
gaccaagcaagacaagcccgtcagcgcaagagaagagctcaggca
aggatcagcagcaaggatgtcatgaagaagatcgcaaact
coRAG1 CDS
gcagcaagatccacctgagcaccaaactgctggccgtggacttcc
ctgagcacttcgtgaagtccatcagctgccagatctgcgagcaca
tcctggccgatcctgtggaaacaaactgcaagcacgtgttctgca
gagtgtgcatcctgcggtgcctgaaagtgatgggcagctactgcc
cctcctgcagatacccttgcttccccaccgatctggaaagccctg
tgaagtccttcctgagcgtgctgaacagcctgatggtcaagtgcc
ccgccaaagaatgcaacgaggaagtgtccctggaaaagtacaacc
accacatcagcagccacaaagagtccaaagaaatcttcgtgcaca
tcaacaaaggcggcagaccccggcagcatctgctgtctcttacaa
gacgggcccagaagcaccggctgagagaactgaagctgcaagtga
aggcctttgccgacaaagaggaaggcggcgacgtcaagagcgtgt
gcatgaccctgtttctgctggccctgagagcccggaatgagcata
gacaggccgatgagctggaagccatcatgcaaggcaaaggcagcg
gactgcagcctgctgtgtgtctggctatcagagtgaacaccttcc
tgtcctgcagccagtaccacaagatgtaccggaccgtgaaggcca
ttaccggcagacagatcttccagcctctgcacgccctgagaaacg
ccgagaaagttctgctgcctggctaccaccacttcgagtggcagc
ctccactgaagaacgtgtccagcagcaccgacgtgggcatcatcg
atggactgagcggactgtctagcagcgtggacgactaccccgtgg
acacaatcgccaagcggttcagatacgacagcgccctggtgtctg
ccctgatggacatggaagaggacatcctggaaggcatgcggagcc
aggacctggacgattacctgaacggccctttcaccgtggtggtca
aagaaagctgtgacggcatgggcgacgtgtccgagaaacacggat
ctggacctgtggtgccagagaaggccgtgcggttcagcttcacca
tcatgaagatcactatcgcccacagcagccagaacgtgaaagtgt
tcgaggaagccaagcctaacagcgagctgtgctgcaagcctctgt
gtctgatgctggccgacgagagcgatcacgagacactgaccgcca
ttctgagccctctgatcgccgaacgggaagccatgaagtcctccg
agctgatgctcgaactcggcggcatcctgagaaccttcaagttca
tcttccgcggcaccggctacgacgagaagctcgttagagaggtgg
aaggcctggaagcctctggcagcgtgtacatctgcaccctgtgtg
acgccaccagactggaagctagccagaacctggtgttccacagca
tcaccagaagccacgccgaaaacctggaaagatacgaagtgtggc
ggagcaacccctaccacgagagcgtggaagaactgcgggatagag
tgaagggcgtgtccgccaagcctttcatcgagacagtgcctagca
tcgacgccctgcactgcgatattggcaacgccgccgaattctaca
agatctttcagctggaaatcggcgaggtgtacaagaaccccaacg
cctctaaagaggaacggaagcgctggcaggccacactggataagc
acctgagaaagaagatgaatctgaagcccatcatgaggatgaacg
gcaacttcgcccggaagctgatgaccaaagaaaccgtggatgccg
tgtgcgagctgatcccctctgaggaaagacacgaggccctgcggg
aactgatggacctgtacctgaagatgaagcccgtgtggcggtcta
gctgtcctgccaaagagtgccctgagtctctgtgccagtacagct
tcaacagccagagattcgccgagctgctgtccaccaagttcaagt
acagatacgagggcaagatcaccaactacttccacaagaccctgg
ctcacgtgcccgagatcatcgagagagatggctctattggcgcct
gggcctctgagggcaatgagtctggcaacaagctgttccggcggt
tccgcaagatgaacgccagacagagcaagtgctacgagatggaag
atgtgctgaagcaccactggctgtacaccagcaagtacctgcaga
aattcatgaacgcccacaacgccctcaagaccagcggctttacca
tgaatcctcaggccagcctgggcgatccttt
Right HA
aggcatagaggactctctggaaagccaagattcaatggaatttta
agtagggcaaccacttatgagttggtttttgcaattgagtttccc
tctgggttgcattgagggcttctcctagcaccctttactgctgtg
tatggggcttcaccatccaagaggtggtaggttggagtaagatgc
tacagatgctctcaagtcaggaatagaaactgatgagctgattgc
ttgaggcttttagtgagttccgaaaagcaacaggaaaaatcagtt
atctgaaagctcagtaactcagaacaggagtaactgcaggggacc
agagatgagcaaagatctgtgtgtgttggggagctgtcatgtaaa
tcaaagccaaggttgtcaaagaacagccagtgaggccaggaaaga
aattggtcttgtggttttcatttttttcccccttgattgattata
ttttgtattgagatatgataagtgccttctatttcatttttgaat
aattcttcatttttataattttacatatcttggcttgctatataa
gattcaaaagagctttttaaatttttctaataatatcttacattt
gtacagcatgatgacctttacaaagtgctctcaatgcatttaccc
attcgttatataaatatgttacatcaggacaactttgagaaaatc
agtccttttttatgtttaaattatgtatctattgtaaccttcaga
gtttaggaggtcatctgctgtcatggatttttcaataatgaattt
agaatacacctgttagctacagttagttattaaatcttctgataa
tatatgtttacttagctatcagaagccaagtatgattctttattt
ttactttttcatttcaagaaatttagagtttccaaatttagagct
DONOR specific for “g6 M2 ex2 RAG1”
gRNA for the exon 2 RAG1 gene targeting
strategy
INSERT
tgagatcctttgaaaagacacctgaagaagctcaaaaggaaaaga
aggattcctttgaggggaaaccctctctggagcaatctccagcag
tcctggacaaggctgatggtcagaagccagtcccaactcagccat
tgttaaaagcccaccctaagttttcaaagaaatttcacgacaacg
agaaagcaagaggcaaagcgatccatcaagccaaccttcgacatc
tctgccgcatctgtgggaattcttttagagctgatgagcacaaca
ggagatatccagtccatggtcctgtggatggtaaaaccctaggcc
ttttacgaaagaaggaaaagagagctacttcctggccggacctca
ttgccaaggttttccggatcgatgtgaaggcagatgttgactcga
tccaccccactgagttctgccataactgctggagcatcatgcaca
ggaagtttagcagtgccccatgtgaggtttacttcccgaggaatg
tcactatggaatggcaccctcacacacccagctgcgacatctgca
acacagccagaagaggcctgaagcggaagtccctgcagcctaatc
tgcagctgagcaagaaactgaaaaccgtgctggaccaggccagac
aggcccggcaaagaaagagaagggcccaagccagaatcagcagca
aggacgtgatgaagaagatcgccaactgcagcaagatccacctga
gcaccaaactgctggccgtggacttccctgagcacttcgtgaagt
ccatcagctgccagatctgcgagcacatcctggccgatcctgtgg
aaacaaactgcaagcacgtgttctgcagagtgtgcatcctgcggt
gcctgaaagtgatgggcagctactgcccctcctgcagataccctt
gcttccccaccgatctggaaagccctgtgaagtccttcctgagcg
tgctgaacagcctgatggtcaagtgccccgccaaagaatgcaacg
aggaagtgtccctggaaaagtacaaccaccacatcagcagccaca
aagagtccaaagaaatcttcgtgcacatcaacaaaggcggcagac
cccggcagcatctgctgtctcttacaagacgggcccagaagcacc
ggctgagagaactgaagctgcaagtgaaggcctttgccgacaaag
aggaaggcggcgacgtcaagagcgtgtgcatgaccctgtttctgc
tggccctgagagcccggaatgagcatagacaggccgatgagctgg
aagccatcatgcaaggcaaaggcagcggactgcagcctgctgtgt
gtctggctatcagagtgaacaccttcctgtcctgcagccagtacc
acaagatgtaccggaccgtgaaggccattaccggcagacagatct
tccagcctctgcacgccctgagaaacgccgagaaagttctgctgc
ctggctaccaccacttcgagtggcagcctccactgaagaacgtgt
ccagcagcaccgacgtgggcatcatcgatggactgagcggactgt
ctagcagcgtggacgactaccccgtggacacaatcgccaagcggt
tcagatacgacagcgccctggtgtctgccctgatggacatggaag
aggacatcctggaaggcatgcggagccaggacctggacgattacc
tgaacggccctttcaccgtggtggtcaaagaaagctgtgacggca
tgggcgacgtgtccgagaaacacggatctggacctgtggtgccag
agaaggccgtgcggttcagcttcaccatcatgaagatcactatcg
cccacagcagccagaacgtgaaagtgttcgaggaagccaagccta
acagcgagctgtgctgcaagcctctgtgtctgatgctggccgacg
agagcgatcacgagacactgaccgccattctgagccctctgatcg
ccgaacgggaagccatgaagtcctccgagctgatgctcgaactcg
gcggcatcctgagaaccttcaagttcatcttccgcggcaccggct
acgacgagaagctcgttagagaggtggaaggcctggaagcctctg
gcagcgtgtacatctgcaccctgtgtgacgccaccagactggaag
ctagccagaacctggtgttccacagcatcaccagaagccacgccg
aaaacctggaaagatacgaagtgtggcggagcaacccctaccacg
agagcgtggaagaactgcgggatagagtgaagggcgtgtccgcca
agcctttcatcgagacagtgcctagcatcgacgccctgcactgcg
atattggcaacgccgccgaattctacaagatctttcagctggaaa
tcggcgaggtgtacaagaaccccaacgcctctaaagaggaacgga
agcgctggcaggccacactggataagcacctgagaaagaagatga
atctgaagcccatcatgaggatgaacggcaacttcgcccggaagc
tgatgaccaaagaaaccgtggatgccgtgtgcgagctgatcccct
ctgaggaaagacacgaggccctgcgggaactgatggacctgtacc
tgaagatgaagcccgtgtggcggtctagctgtcctgccaaagagt
gccctgagtctctgtgccagtacagcttcaacagccagagattcg
ccgagctgctgtccaccaagttcaagtacagatacgagggcaaga
tcaccaactacttccacaagaccctggctcacgtgcccgagatca
tcgagagagatggctctattggcgcctgggcctctgagggcaatg
agtctggcaacaagctgttccggcggttccgcaagatgaacgcca
gacagagcaagtgctacgagatggaagatgtgctgaagcaccact
ggctgtacaccagcaagtacctgcagaaattcatgaacgcccaca
acgccctcaagaccagcggctttaccatgaatcctcaggccagcc
tgggcgaccctctgggaattgaggatagcctggaatcccaggaca
gcatggaattctgataagagtggcacccccacacaccatcctgtg
acatctgcaacactgcccgtcggggactcaagaggaagagtcttc
agccaaacttgcagctcagcaaaaaactcaaaactgtgcttgacc
aagcaagacaagcccgtcagcgcaagagaagagctcaggcaagga
tcagcagcaaggatgtcatgaagaagatcgccaactgcagtaaga
tacatcttagtaccaagctccttgcagtggacttcccagagc
Left HA
tgagatcctttgaaaagacacctgaagaagctcaaaaggaaaaga
aggattcctttgaggggaaaccctctctggagcaatctccagcag
tcctggacaaggctgatggtcagaagccagtcccaactcagccat
tgttaaaagcccaccctaagttttcaaagaaatttcacgacaacg
agaaagcaagaggcaaagcgatccatcaagccaaccttcgacatc
tctgccgcatctgtgggaattcttttagagctgatgagcacaaca
ggagatatccagtccatggtcctgtggatggtaaaaccctaggcc
ttttacgaaagaaggaaaagagagctacttcctggccggacctca
ttgccaaggttttccggatcgatgtgaaggcagatgttgactcga
tccaccccactgagttctgccataactgctggagcatcatgcaca
ggaagtttagcagtgccccatgtgaggtttacttcccgaggaatg
tcactatg
coRAG1 CDS
gaatggcaccctcacacacccagctgcgacatctgcaacacagcc
agaagaggcctgaagcggaagtccctgcagcctaatctgcagctg
agcaagaaactgaaaaccgtgctggaccaggccagacaggcccgg
caaagaaagagaagggcccaagccagaatcagcagcaaggacgtg
atgaagaagatcgccaactgcagcaagatccacctgagcaccaaa
ctgctggccgtggacttccctgagcacttcgtgaagtccatcagc
tgccagatctgcgagcacatcctggccgatcctgtggaaacaaac
tgcaagcacgtgttctgcagagtgtgcatcctgcggtgcctgaaa
gtgatgggcagctactgcccctcctgcagatacccttgcttcccc
accgatctggaaagccctgtgaagtccttcctgagcgtgctgaac
agcctgatggtcaagtgccccgccaaagaatgcaacgaggaagtg
tccctggaaaagtacaaccaccacatcagcagccacaaagagtcc
aaagaaatcttcgtgcacatcaacaaaggcggcagaccccggcag
catctgctgtctcttacaagacgggcccagaagcaccggctgaga
gaactgaagctgcaagtgaaggcctttgccgacaaagaggaaggc
ggcgacgtcaagagcgtgtgcatgaccctgtttctgctggccctg
agagcccggaatgagcatagacaggccgatgagctggaagccatc
atgcaaggcaaaggcagcggactgcagcctgctgtgtgtctggct
atcagagtgaacaccttcctgtcctgcagccagtaccacaagatg
taccggaccgtgaaggccattaccggcagacagatcttccagcct
ctgcacgccctgagaaacgccgagaaagttctgctgcctggctac
caccacttcgagtggcagcctccactgaagaacgtgtccagcagc
accgacgtgggcatcatcgatggactgagcggactgtctagcagc
gtggacgactaccccgtggacacaatcgccaagcggttcagatac
gacagcgccctggtgtctgccctgatggacatggaagaggacatc
ctggaaggcatgcggagccaggacctggacgattacctgaacggc
cctttcaccgtggtggtcaaagaaagctgtgacggcatgggcgac
gtgtccgagaaacacggatctggacctgtggtgccagagaaggcc
gtgcggttcagcttcaccatcatgaagatcactatcgcccacagc
agccagaacgtgaaagtgttcgaggaagccaagcctaacagcgag
ctgtgctgcaagcctctgtgtctgatgctggccgacgagagcgat
cacgagacactgaccgccattctgagccctctgatcgccgaacgg
gaagccatgaagtcctccgagctgatgctcgaactcggcggcatc
ctgagaaccttcaagttcatcttccgcggcaccggctacgacgag
aagctcgttagagaggtggaaggcctggaagcctctggcagcgtg
tacatctgcaccctgtgtgacgccaccagactggaagctagccag
aacctggtgttccacagcatcaccagaagccacgccgaaaacctg
gaaagatacgaagtgtggcggagcaacccctaccacgagagcgtg
gaagaactgcgggatagagtgaagggcgtgtccgccaagcctttc
atcgagacagtgcctagcatcgacgccctgcactgcg atattgg
caacgccgccgaattctacaagatctttcagctggaaatcggcga
ggtgtacaagaaccccaacgcctctaaagaggaacggaagcgctg
gcaggccacactggataagcacctgagaaagaagatgaatctgaa
gcccatcatgaggatgaacggcaacttcgcccggaagctgatgac
caaagaaaccgtggatgccgtgtgcgagctgatcccctctgagga
aagacacgaggccctgcgggaactgatggacctgtacctgaagat
gaagcccgtgtggcggtctagctgtcctgccaaagagtgccctga
gtctctgtgccagtacagcttcaacagccagagattcgccgagct
gctgtccaccaagttcaagtacagatacgagggcaagatcaccaa
ctacttccacaagaccctggctcacgtgcccgagatcatcgagag
agatggctctattggcgcctgggcctctgagggcaatgagtctgg
caacaagctgttccggcggttccgcaagatgaacgccagacagag
caagtgctacgagatggaagatgtgctgaagcaccactggctgta
caccagcaagtacctgcagaaattcatgaacgcccacaacgccct
caagaccagcggctttaccatgaatcctcaggccagcctgggcga
ccctctgggaattgaggatagcctggaatcccaggacagcatgga
attctgataa
Right HA
gagtggcacccccacacaccatcctgtgacatctgcaacactgcc
cgtcggggactcaagaggaagagtcttcagccaaacttgcagctc
agcaaaaaactcaaaactgtgcttgaccaagcaagacaagcccgt
cagcgcaagagaagagctcaggcaaggatcagcagcaaggatgtc
atgaagaagatcgccaactgcagtaagatacatcttagtaccaag
ctccttgcagtggacttcccagagc
DONOR specific for “g6 M2 ex2 RAG1”
gRNA for the exon 2 RAG1 gene replacement
strategy with long right HA
INSERT
gagcacaacaggagatatccagtccatggtcctgtggatggtaaa
accctaggccttttacgaaagaaggaaaagagagctacttcctgg
ccggacctcattgccaaggttttccggatcgatgtgaaggcagat
gttgactcgatccaccccactgagttctgccataactgctggagc
atcatgcacaggaagtttagcagtgccccatgtgaggtttacttc
ccgaggaatgtcactatggaatggcaccctcacacacccagctgc
gacatctgcaacacagccagaagaggcctgaagcggaagtccctg
cagcctaatctgcagctgagcaagaaactgaaaaccgtgctggac
caggccagacaggcccggcaaagaaagagaagggcccaagccaga
atcagcagcaaggacgtgatgaagaagatcgccaactgcagcaag
atccacctgagcaccaaactgctggccgtggacttccctgagcac
ttcgtgaagtccatcagctgccagatctgcgagcacatcctggcc
gatcctgtggaaacaaactgcaagcacgtgttctgcagagtgtgc
atcctgcggtgcctgaaagtgatgggcagctactgcccctcctgc
agatacccttgcttccccaccgatctggaaagccctgtgaagtcc
ttcctgagcgtgctgaacagcctgatggtcaagtgccccgccaaa
gaatgcaacgaggaagtgtccctggaaaagtacaaccaccacatc
agcagccacaaagagtccaaagaaatcttcgtgcacatcaacaaa
ggcggcagaccccggcagcatctgctgtctcttacaagacgggcc
cagaagcaccggctgagagaactgaagctgcaagtgaaggccttt
gccgacaaagaggaaggcggcgacgtcaagagcgtgtgcatgacc
ctgtttctgctggccctgagagcccggaatgagcatagacaggcc
gatgagctggaagccatcatgcaaggcaaaggcagcggactgcag
cctgctgtgtgtctggctatcagagtgaacaccttcctgtcctgc
agccagtaccacaagatgtaccggaccgtgaaggccattaccggc
agacagatcttccagcctctgcacgccctgagaaacgccgagaaa
gttctgctgcctggctaccaccacttcgagtggcagcctccactg
aagaacgtgtccagcagcaccgacgtgggcatcatcgatggactg
agcggactgtctagcagcgtggacgactaccccgtggacacaatc
gccaagcggttcagatacgacagcgccctggtgtctgccctgatg
gacatggaagaggacatcctggaaggcatgcggagccaggacctg
gacgattacctgaacggccctttcaccgtggtggtcaaagaaagc
tgtgacggcatgggcgacgtgtccgagaaacacggatctggacct
gtggtgccagagaaggccgtgcggttcagcttcaccatcatgaag
atcactatcgcccacagcagccagaacgtgaaagtgttcgaggaa
gccaagcctaacagcgagctgtgctgcaagcctctgtgtctgatg
ctggccgacgagagcgatcacgagacactgaccgccattctgagc
cctctgatcgccgaacgggaagccatgaagtcctccgagctgatg
ctcgaactcggcggcatcctgagaaccttcaagttcatcttccgc
ggcaccggctacgacgagaagctcgttagagaggtggaaggcctg
gaagcctctggcagcgtgtacatctgcaccctgtgtgacgccacc
agactggaagctagccagaacctggtgttccacagcatcaccaga
agccacgccgaaaacctggaaagatacgaagtgtggcggagcaac
ccctaccacgagagcgtggaagaactgcgggatagagtgaagggc
gtgtccgccaagcctttcatcgagacagtgcctagcatcgacgcc
ctgcactgcgatattggcaacgccgccgaattctacaagatcttt
cagctggaaatcggcgaggtgtacaagaaccccaacgcctctaaa
gaggaacggaagcgctggcaggccacactggataagcacctgaga
aagaagatgaatctgaagcccatcatgaggatgaacggcaacttc
gcccggaagctgatgaccaaagaaaccgtggatgccgtgtgcgag
ctgatcccctctgaggaaagacacgaggccctgcgggaactgatg
gacctgtacctgaagatgaagcccgtgtggcggtctagctgtcct
gccaaagagtgccctgagtctctgtgccagtacagcttcaacagc
cagagattcgccgagctgctgtccaccaagttcaagtacagatac
gagggcaagatcaccaactacttccacaagaccctggctcacgtg
cccgagatcatcgagagagatggctctattggcgcctgggcctct
gagggcaatgagtctggcaacaagctgttccggcggttccgcaag
atgaacgccagacagagcaagtgctacgagatggaagatgtgctg
aagcaccactggctgtacaccagcaagtacctgcagaaattcatg
aacgcccacaacgccctcaagaccagcggctttaccatgaatcct
caggccagcctgggcgatcctttaggcatagaggactctctggaa
agccaagattcaatggaattttaagtagggcaaccacttatgagt
tggtttttgcaattgagtttccctctgggttgcattgagggcttc
tcctagcaccctttactgctgtgtatggggcttcaccatccaaga
ggtggtaggttggagtaagatgctacagatgctctcaagtcagga
atagaaactgatgagctgattgcttgaggcttttagtgagttccg
aaaagcaacaggaaaaatcagttatctgaaagctcagtaactcag
aacaggagtaactgcaggggaccagagatgagcaaagatctgtgt
gtgttggggagctgtcatgtaaatcaaagccaaggttgtcaaaga
acagccagtgaggccaggaaagaaattggtcttgtggttttcatt
tttttcccccttgattgattatattttgtattgagatatgataag
tgccttctatttcatttttgaataattcttcatttttataatttt
acatatcttggcttgctatataagattcaaaagagctttttaaat
ttttctaataatatcttacatttgtacagcatgatgacctttaca
aagtgctctcaatgcatttacccattcgttatataaatatgttac
atcaggacaactttgagaaaatcagtccttttttatgtttaaatt
atgtatctattgtaaccttcagagtttaggaggtcatctgctgtc
atggatttttcaataatgaatttagaatacacctgttagctacag
ttagttattaaatcttctgataatatatgtttacttagctatcag
aagccaagtatgattctttatttttactttttcatttcaagaaat
ttagagtttccaaatttagagcttctgcatacagtcttaaagcca
cagaggcttgtaaaaatataggttagcttgatgtctaaaaatata
tttcatgtcttactgaaacattttgccagactttctccaaatgaa
acctgaatcaatttttctaaatctaggtttcatagagtcctctcc
tctgcaatgtgttattctttctataatgatcagtttactttcagt
ggattcagaattgtgtagcaggataaccttgtatttttccatccg
ctaagtttagatggagtccaaacgcagtacagcagaagagttaac
atttacacagtgctttttaccactgtggaatgttttcacactcat
ttttccttacaacaattctgaggagtaggtgttgttattatctcc
atttgatgggggtttaaatgatttgctcaaagtcatttaggggta
ataaatacttggcttggaaatttaacacagtccttttgtctccaa
agcccttcttctttccaccacaaattaatcactatgtttataagg
tagtatcagaatttttttaggattcacaactaatcactatagcac
atgaccttgggattacatttttatggggcaggggtaagcaagttt
ttaaatcatttgtgtgctctggctcttttgatagaagaaagcaac
acaaaagctccaaagggccccctaaccctcttgtggctccagtta
tttggaaactatgatctgcatccttaggaatctgggatttgccag
ttgctggcaatgtagagcaggcatggaattttatatgctagtgag
tcataatgatatgttagtgttaattagttttttcttcctttgatt
ttattggccataattgctactcttcatacacagtatatcaaagag
cttgataatttagttgtcaaaag
Left HA
gagcacaacaggagatatccagtccatggtcctgtggatggtaaa
accctaggccttttacgaaagaaggaaaagagagctacttcctgg
ccggacctcattgccaaggttttccggatcgatgtgaaggcagat
gttgactcgatccaccccactgagttctgccataactgctggagc
atcatgcacaggaagtttagcagtgccccatgtgaggtttacttc
ccgaggaatgtcactatg
coRAG1 CDS
gaatggcaccctcacacacccagctgcgacatctgcaacacagcc
agaagaggcctgaagcggaagtccctgcagcctaatctgcagctg
agcaagaaactgaaaaccgtgctggaccaggccagacaggcccgg
caaagaaagagaagggcccaagccagaatcagcagcaaggacgtg
atgaagaagatcgccaactgcagcaagatccacctgagcaccaaa
ctgctggccgtggacttccctgagcacttcgtgaagtccatcagc
tgccagatctgcgagcacatcctggccgatcctgtggaaacaaac
tgcaagcacgtgttctgcagagtgtgcatcctgcggtgcctgaaa
gtgatgggcagctactgcccctcctgcagatacccttgcttcccc
accgatctggaaagccctgtgaagtccttcctgagcgtgctgaac
agcctgatggtcaagtgccccgccaaagaatgcaacgaggaagtg
tccctggaaaagtacaaccaccacatcagcagccacaaagagtcc
aaagaaatcttcgtgcacatcaacaaaggcggcagaccccggcag
catctgctgtctcttacaagacgggcccagaagcaccggctgaga
gaactgaagctgcaagtgaaggcctttgccgacaaagaggaaggc
ggcgacgtcaagagcgtgtgcatgaccctgtttctgctggccctg
agagcccggaatgagcatagacaggccgatgagctggaagccatc
atgcaaggcaaaggcagcggactgcagcctgctgtgtgtctggct
atcagagtgaacaccttcctgtcctgcagccagtaccacaagatg
taccggaccgtgaaggccattaccggcagacagatcttccagcct
ctgcacgccctgagaaacgccgagaaagttctgctgcctggctac
caccacttcgagtggcagcctccactgaagaacgtgtccagcagc
accgacgtgggcatcatcgatggactgagcggactgtctagcagc
gtggacgactaccccgtggacacaatcgccaagcggttcagatac
gacagcgccctggtgtctgccctgatggacatggaagaggacatc
ctggaaggcatgcggagccaggacctggacgattacctgaacggc
cctttcaccgtggtggtcaaagaaagctgtgacggcatgggcgac
gtgtccgagaaacacggatctggacctgtggtgccagagaaggcc
gtgcggttcagcttcaccatcatgaagatcactatcgcccacagc
agccagaacgtgaaagtgttcgaggaagccaagcctaacagcgag
ctgtgctgcaagcctctgtgtctgatgctggccgacgagagcgat
cacgagacactgaccgccattctgagccctctgatcgccgaacgg
gaagccatgaagtcctccgagctgatgctcgaactcggcggcatc
ctgagaaccttcaagttcatcttccgcggcaccggctacgacgag
aagctcgttagagaggtggaaggcctggaagcctctggcagcgtg
tacatctgcaccctgtgtgacgccaccagactggaagctagccag
aacctggtgttccacagcatcaccagaagccacgccgaaaacctg
gaaagatacgaagtgtggcggagcaacccctaccacgagagcgtg
gaagaactgcgggatagagtgaagggcgtgtccgccaagcctttc
atcgagacagtgcctagcatcgacgccctgcactgcg atattgg
caacgccgccgaattctacaagatctttcagctggaaatcggcga
ggtgtacaagaaccccaacgcctctaaagaggaacggaagcgctg
gcaggccacactggataagcacctgagaaagaagatgaatctgaa
gcccatcatgaggatgaacggcaacttcgcccggaagctgatgac
caaagaaaccgtggatgccgtgtgcgagctgatcccctctgagga
aagacacgaggccctgcgggaactgatggacctgtacctgaagat
gaagcccgtgtggcggtctagctgtcctgccaaagagtgccctga
gtctctgtgccagtacagcttcaacagccagagattcgccgagct
gctgtccaccaagttcaagtacagatacgagggcaagatcaccaa
ctacttccacaagaccctggctcacgtgcccgagatcatcgagag
agatggctctattggcgcctgggcctctgagggcaatgagtctgg
caacaagctgttccggcggttccgcaagatgaacgccagacagag
caagtgctacgagatggaagatgtgctgaagcaccactggctgta
caccagcaagtacctgcagaaattcatgaacgcccacaacgccct
caagaccagcggctttaccatgaatcctcaggccagcctgggcga
tccttt
Right HA
aggcatagaggactctctggaaagccaagattcaatggaatttta
agtagggcaaccacttatgagttg gtttttgcaattgagtttcc
ctctgggttgcattgagggcttctcctagcaccctttactgctgt
gtatggggcttcaccatccaagaggtggtaggttggagtaagatg
ctacagatgctctcaagtcaggaatagaaactgatgagctgattg
cttgaggcttttagtgagttccgaaaagcaacaggaaaaatcagt
tatctgaaagctcagtaactcagaacaggagtaactgcaggggac
cagagatgagcaaagatctgtgtgtgttggggagctgtcatgtaa
atcaaagccaaggttgtcaaagaacagccagtgaggccaggaaag
aaattggtcttgtggttttcatttttttcccccttgattgattat
attttgtattgagatatgataagtgccttctatttcatttttgaa
taattcttcatttttataattttacatatcttggcttgctatata
agattcaaaagagctttttaaatttttctaataatatcttacatt
tgtacagcatgatgacctttacaaagtgctctcaatgcatttacc
cattcgttatataaatatgttacatcaggacaactttgagaaaat
cagtccttttttatgtttaaattatgtatctattgtaaccttcag
agtttaggaggtcatctgctgtcatggatttttcaataatgaatt
tagaatacacctgttagctacagttagttattaaatcttctgata
atatatgtttacttagctatcagaagccaagtatgattctttatt
tttactttttcatttcaagaaatttagagtttccaaatttagagc
ttctgcatacagtcttaaagccacagaggcttgtaaaaatatagg
ttagcttgatgtctaaaaatatatttcatgtcttactgaaacatt
ttgccagactttctccaaatgaaacctgaatcaatttttctaaat
ctaggtttcatagagtcctctcctctgcaatgtgttattctttct
ataatgatcagtttactttcagtggattcagaattgtgtagcagg
ataaccttgtatttttccatccgctaagtttagatggagtccaaa
cgcagtacagcagaagagttaacatttacacagtgctttttacca
ctgtggaatgttttcacactcatttttccttacaacaattctgag
gagtaggtgttgttattatctccatttgatgggggtttaaatgat
ttgctcaaagtcatttaggggtaataaatacttggcttggaaatt
taacacagtccttttgtctccaaagcccttcttctttccaccaca
aattaatcactatgtttataaggtagtatcagaatttttttagga
ttcacaactaatcactatagcacatgaccttgggattacattttt
atggggcaggggtaagcaagtttttaaatcatttgtgtgctctgg
ctcttttgatagaagaaagcaacacaaaagctccaaagggccccc
taaccctcttgtggctccagttatttggaaactatgatctgcatc
cttaggaatctgggatttgccagttgctggcaatgtagagcaggc
atggaattttatatgctagtgagtcataatgatatgttagtgtta
attagttttttcttcctttgattttattggccataattgctactc
ttcatacacagtatatcaaagagcttgataatttagttgtcaaaa
g
DONOR specific for “g6 M2 ex2 RAG1”
gRNA for the exon 2 RAG1 gene replacement
strategy with short right HA
INSERT
gagcacaacaggagatatccagtccatggtcctgtggatggtaaa
accctaggccttttacgaaagaaggaaaagagagctacttcctgg
ccggacctcattgccaaggttttccggatcgatgtgaaggcagat
gttgactcgatccaccccactgagttctgccataactgctggagc
atcatgcacaggaagtttagcagtgccccatgtgaggtttacttc
ccgaggaatgtcactatggaatggcaccctcacacacccagctgc
gacatctgcaacacagccagaagaggcctgaagcggaagtccctg
cagcctaatctgcagctgagcaagaaactgaaaaccgtgctggac
caggccagacaggcccggcaaagaaagagaagggcccaagccaga
atcagcagcaaggacgtgatgaagaagatcgccaactgcagcaag
atccacctgagcaccaaactgctggccgtggacttccctgagcac
ttcgtgaagtccatcagctgccagatctgcgagcacatcctggcc
gatcctgtggaaacaaactgcaagcacgtgttctgcagagtgtgc
atcctgcggtgcctgaaagtgatgggcagctactgcccctcctgc
agatacccttgcttccccaccgatctggaaagccctgtgaagtcc
ttcctgagcgtgctgaacagcctgatggtcaagtgccccgccaaa
gaatgcaacgaggaagtgtccctggaaaagtacaaccaccacatc
agcagccacaaagagtccaaagaaatcttcgtgcacatcaacaaa
ggcggcagaccccggcagcatctgctgtctcttacaagacgggcc
cagaagcaccggctgagagaactgaagctgcaagtgaaggccttt
gccgacaaagaggaaggcggcgacgtcaagagcgtgtgcatgacc
ctgtttctgctggccctgagagcccggaatgagcatagacaggcc
gatgagctggaagccatcatgcaaggcaaaggcagcggactgcag
cctgctgtgtgtctggctatcagagtgaacaccttcctgtcctgc
agccagtaccacaagatgtaccggaccgtgaaggccattaccggc
agacagatcttccagcctctgcacgccctgagaaacgccgagaaa
gttctgctgcctggctaccaccacttcgagtggcagcctccactg
aagaacgtgtccagcagcaccgacgtgggcatcatcgatggactg
agcggactgtctagcagcgtggacgactaccccgtggacacaatc
gccaagcggttcagatacgacagcgccctggtgtctgccctgatg
gacatggaagaggacatcctggaaggcatgcggagccaggacctg
gacgattacctgaacggccctttcaccgtggtggtcaaagaaagc
tgtgacggcatgggcgacgtgtccgagaaacacggatctggacct
gtggtgccagagaaggccgtgcggttcagcttcaccatcatgaag
atcactatcgcccacagcagccagaacgtgaaagtgttcgaggaa
gccaagcctaacagcgagctgtgctgcaagcctctgtgtctgatg
ctggccgacgagagcgatcacgagacactgaccgccattctgagc
cctctgatcgccgaacgggaagccatgaagtcctccgagctgatg
ctcgaactcggcggcatcctgagaaccttcaagttcatcttccgc
ggcaccggctacgacgagaagctcgttagagaggtggaaggcctg
gaagcctctggcagcgtgtacatctgcaccctgtgtgacgccacc
agactggaagctagccagaacctggtgttccacagcatcaccaga
agccacgccgaaaacctggaaagatacgaagtgtggcggagcaac
ccctaccacgagagcgtggaagaactgcgggatagagtgaagggc
gtgtccgccaagcctttcatcgagacagtgcctagcatcgacgcc
ctgcactgcgatattggcaacgccgccgaattctacaagatcttt
cagctggaaatcggcgaggtgtacaagaaccccaacgcctctaaa
gaggaacggaagcgctggcaggccacactggataagcacctgaga
aagaagatgaatctgaagcccatcatgaggatgaacggcaacttc
gcccggaagctgatgaccaaagaaaccgtggatgccgtgtgcgag
ctgatcccctctgaggaaagacacgaggccctgcgggaactgatg
gacctgtacctgaagatgaagcccgtgtggcggtctagctgtcct
gccaaagagtgccctgagtctctgtgccagtacagcttcaacagc
cagagattcgccgagctgctgtccaccaagttcaagtacagatac
gagggcaagatcaccaactacttccacaagaccctggctcacgtg
cccgagatcatcgagagagatggctctattggcgcctgggcctct
gagggcaatgagtctggcaacaagctgttccggcggttccgcaag
atgaacgccagacagagcaagtgctacgagatggaagatgtgctg
aagcaccactggctgtacaccagcaagtacctgcagaaattcatg
aacgcccacaacgccctcaagaccagcggctttaccatgaatcct
caggccagcctgggcgatcctttaggcatagaggactctctggaa
agccaagattcaatggaattttaagtagggcaaccacttatgagt
tggtttttgcaattgagtttccctctgggttgcattgagggcttc
tcctagcaccctttactgctgtgtatggggcttcaccatccaaga
ggtggtaggttggagtaagatgctacagatgctctcaagtcagga
atagaaactgatgagctgattgcttgaggcttttagtgagttccg
aaaagcaacaggaaaaatcagttatctgaaagctcagtaactcag
aacaggagtaactgcaggggaccagagatgagcaaagatctgtgt
gtgttggggagctgtcatgtaaatcaaagccaaggttgtcaaaga
acagccagtgaggccaggaaagaaattggtcttgtggttttcatt
tttttcccccttgattgattatattttgtattgagatatgataag
tgccttctatttcatttttgaataattcttcatttttataatttt
acatatcttggcttgctatataagattcaaaagagctttttaaat
ttttctaataatatcttacatttgtacagcatgatgacctttaca
aagtgctctcaatgcatttacccattcgttatataaatatgttac
atcaggacaactttgagaaaatcagtccttttttatgtttaaatt
atgtatctattgtaaccttcagagtttaggaggtcatctgctgtc
atggatttttcaataatgaatttagaatacacctgttagctacag
ttagttattaaatcttctgataatatatgtttacttagctatcag
aagccaagtatgattctttatttttactttttcatttcaagaaat
ttagagtttccaaatttagagct
Left HA
gagcacaacaggagatatccagtccatggtcctgtggatggtaaa
accctaggccttttacgaaagaaggaaaagagagctacttcctgg
ccggacctcattgccaaggttttccggatcgatgtgaaggcagat
gttgactcgatccaccccactgagttctgccataactgctggagc
atcatgcacaggaagtttagcagtgccccatgtgaggtttacttc
ccgaggaatgtcactatg
coRAG1 CDS
gaatggcaccctcacacacccagctgcgacatctgcaacacagcc
agaagaggcctgaagcggaagtccctgcagcctaatctgcagctg
agcaagaaactgaaaaccgtgctggaccaggccagacaggcccgg
caaagaaagagaagggcccaagccagaatcagcagcaaggacgtg
atgaagaagatcgccaactgcagcaagatccacctgagcaccaaa
ctgctggccgtggacttccctgagcacttcgtgaagtccatcagc
tgccagatctgcgagcacatcctggccgatcctgtggaaacaaac
tgcaagcacgtgttctgcagagtgtgcatcctgcggtgcctgaaa
gtgatgggcagctactgcccctcctgcagatacccttgcttcccc
accgatctggaaagccctgtgaagtccttcctgagcgtgctgaac
agcctgatggtcaagtgccccgccaaagaatgcaacgaggaagtg
tccctggaaaagtacaaccaccacatcagcagccacaaagagtcc
aaagaaatcttcgtgcacatcaacaaaggcggcagaccccggcag
catctgctgtctcttacaagacgggcccagaagcaccggctgaga
gaactgaagctgcaagtgaaggcctttgccgacaaagaggaaggc
ggcgacgtcaagagcgtgtgcatgaccctgtttctgctggccctg
agagcccggaatgagcatagacaggccgatgagctggaagccatc
atgcaaggcaaaggcagcggactgcagcctgctgtgtgtctggct
atcagagtgaacaccttcctgtcctgcagccagtaccacaagatg
taccggaccgtgaaggccattaccggcagacagatcttccagcct
ctgcacgccctgagaaacgccgagaaagttctgctgcctggctac
caccacttcgagtggcagcctccactgaagaacgtgtccagcagc
accgacgtgggcatcatcgatggactgagcggactgtctagcagc
gtggacgactaccccgtggacacaatcgccaagcggttcagatac
gacagcgccctggtgtctgccctgatggacatggaagaggacatc
ctggaaggcatgcggagccaggacctggacgattacctgaacggc
cctttcaccgtggtggtcaaagaaagctgtgacggcatgggcgac
gtgtccgagaaacacggatctggacctgtggtgccagagaaggcc
gtgcggttcagcttcaccatcatgaagatcactatcgcccacagc
agccagaacgtgaaagtgttcgaggaagccaagcctaacagcgag
ctgtgctgcaagcctctgtgtctgatgctggccgacgagagcgat
cacgagacactgaccgccattctgagccctctgatcgccgaacgg
gaagccatgaagtcctccgagctgatgctcgaactcggcggcatc
ctgagaaccttcaagttcatcttccgcggcaccggctacgacgag
aagctcgttagagaggtggaaggcctggaagcctctggcagcgtg
tacatctgcaccctgtgtgacgccaccagactggaagctagccag
aacctggtgttccacagcatcaccagaagccacgccgaaaacctg
gaaagatacgaagtgtggcggagcaacccctaccacgagagcgtg
gaagaactgcgggatagagtgaagggcgtgtccgccaagcctttc
atcgagacagtgcctagcatcgacgccctgcactgcgatattggc
aacgccgccgaattctacaagatctttcagctggaaatcggcgag
gtgtacaagaaccccaacgcctctaaagaggaacggaagcgctgg
caggccacactggataagcacctgagaaagaagatgaatctgaag
cccatcatgaggatgaacggcaacttcgcccggaagctgatgacc
aaagaaaccgtggatgccgtgtgcgagctgatcccctctgaggaa
agacacgaggccctgcgggaactgatggacctgtacctgaagatg
aagcccgtgtggcggtctagctgtcctgccaaagagtgccctgag
tctctgtgccagtacagcttcaacagccagagattcgccgagctg
ctgtccaccaagttcaagtacagatacgagggcaagatcaccaac
tacttccacaagaccctggctcacgtgcccgagatcatcgagaga
gatggctctattggcgcctgggcctctgagggcaatgagtctggc
aacaagctgttccggcggttccgcaagatgaacgccagacagagc
aagtgctacgagatggaagatgtgctgaagcaccactggctgtac
accagcaagtacctgcagaaattcatgaacgcccacaacgccctc
aagaccagcggctttaccatgaatcctcaggccagcctgggcgat
ccttt
Right HA
aggcatagaggactctctggaaagccaagattcaatggaatttta
agtagggcaaccacttatgagttggtttttgcaattgagtttccc
tctgggttgcattgagggcttctcctagcaccctttactgctgtg
tatggggcttcaccatccaagaggtggtaggttggagtaagatgc
tacagatgctctcaagtcaggaatagaaactgatgagctgattgc
ttgaggcttttagtgagttccgaaaagcaacaggaaaaatcagtt
atctgaaagctcagtaactcagaacaggagtaactgcaggggacc
agagatgagcaaagatctgtgtgtgttggggagctgtcatgtaaa
tcaaagccaaggttgtcaaagaacagccagtgaggccaggaaaga
aattggtcttgtggttttcatttttttcccccttgattgattata
ttttgtattgagatatgataagtgccttctatttcatttttgaat
aattcttcatttttataattttacatatcttggcttgctatataa
gattcaaaagagctttttaaatttttctaataatatcttacattt
gtacagcatgatgacctttacaaagtgctctcaatgcatttaccc
attcgttatataaatatgttacatcaggacaactttgagaaaatc
agtccttttttatgtttaaattatgtatctattgtaaccttcaga
gtttaggaggtcatctgctgtcatggatttttcaataatgaattt
agaatacacctgttagctacagttagttattaaatcttctgataa
tatatgtttacttagctatcagaagccaagtatgattctttattt
ttactttttcatttcaagaaatttagagtttccaaatttagagct
DONOR specific for “g7 exon2 M2/3”,
“g10 exon2 M2/3” and “g13 exon2 M2/3”
gRNAs for the exon 2 RAG1 gene replacement
strategy with long right HA
INSERT
gaattcttttagagctgatgagcacaacaggagatatccagtcca
tggtcctgtggatggtaaaaccctaggccttttacgaaagaagga
aaagagagctacttcctggccggacctcattgccaaggttttccg
gatcgatgtgaaggcagatgttgactcgatccaccccactgagtt
ctgccataactgctggagcatcatgcacaggaagtttagcagtgc
accatgcgaagtgtacttccccagaaacgtgaccatggaatggca
ccctcacacacccagctgcgacatctgcaacacagccagaagagg
cctgaagcggaagtccctgcagcctaatctgcagctgagcaagaa
actgaaaaccgtgctggaccaggccagacaggcccggcaaagaaa
gagaagggcccaagccagaatcagcagcaaggacgtgatgaagaa
gatcgccaactgcagcaagatccacctgagcaccaaactgctggc
cgtggacttccctgagcacttcgtgaagtccatcagctgccagat
ctgcgagcacatcctggccgatcctgtggaaacaaactgcaagca
cgtgttctgcagagtgtgcatcctgcggtgcctgaaagtgatggg
cagctactgcccctcctgcagatacccttgcttccccaccgatct
ggaaagccctgtgaagtccttcctgagcgtgctgaacagcctgat
ggtcaagtgccccgccaaagaatgcaacgaggaagtgtccctgga
aaagtacaaccaccacatcagcagccacaaagagtccaaagaaat
cttcgtgcacatcaacaaaggcggcagaccccggcagcatctgct
gtctcttacaagacgggcccagaagcaccggctgagagaactgaa
gctgcaagtgaaggcctttgccgacaaagaggaaggcggcgacgt
caagagcgtgtgcatgaccctgtttctgctggccctgagagcccg
gaatgagcatagacaggccgatgagctggaagccatcatgcaagg
caaaggcagcggactgcagcctgctgtgtgtctggctatcagagt
gaacaccttcctgtcctgcagccagtaccacaagatgtaccggac
cgtgaaggccattaccggcagacagatcttccagcctctgcacgc
cctgagaaacgccgagaaagttctgctgcctggctaccaccactt
cgagtggcagcctccactgaagaacgtgtccagcagcaccgacgt
gggcatcatcgatggactgagcggactgtctagcagcgtggacga
ctaccccgtggacacaatcgccaagcggttcagatacgacagcgc
cctggtgtctgccctgatggacatggaagaggacatcctggaagg
catgcggagccaggacctggacgattacctgaacggccctttcac
cgtggtggtcaaagaaagctgtgacggcatgggcgacgtgtccga
gaaacacggatctggacctgtggtgccagagaaggccgtgcggtt
cagcttcaccatcatgaagatcactatcgcccacagcagccagaa
cgtgaaagtgttcgaggaagccaagcctaacagcgagctgtgctg
caagcctctgtgtctgatgctggccgacgagagcgatcacgagac
actgaccgccattctgagccctctgatcgccgaacgggaagccat
gaagtcctccgagctgatgctcgaactcggcggcatcctgagaac
cttcaagttcatcttccgcggcaccggctacgacgagaagctcgt
tagagaggtggaaggcctggaagcctctggcagcgtgtacatctg
caccctgtgtgacgccaccagactggaagctagccagaacctggt
gttccacagcatcaccagaagccacgccgaaaacctggaaagata
cgaagtgtggcggagcaacccctaccacgagagcgtggaagaact
gcgggatagagtgaagggcgtgtccgccaagcctttcatcgagac
agtgcctagcatcgacgccctgcactgcgatattggcaacgccgc
cgaattctacaagatctttcagctggaaatcggcgaggtctacaa
gaaccccaacgcctctaaagaggaacggaagcgctggcaggccac
actggataagcacctgagaaagaagatgaatctgaagcccatcat
gaggatgaacggcaacttcgcccggaagctgatgaccaaagaaac
cgtggatgccgtgtgcgagctgatcccctctgaggaaagacacga
ggccctgcgggaactgatggacctgtacctgaagatgaagcccgt
gtggcggtctagctgtcctgccaaagagtgccctgagtctctgtg
ccagtacagcttcaacagccagagattcgccgagctgctgtccac
caagttcaagtacagatacgagggcaagatcaccaactacttcca
caagaccctggctcacgtgcccgagatcatcgagagagatggctc
tattggcgcctgggcctctgagggcaatgagtctggcaacaagct
gttccggcggttccgcaagatgaacgccagacagagcaagtgcta
cgagatggaagatgtgctgaagcaccactggctgtacaccagcaa
gtacctgcagaaattcatgaacgcccacaacgccctcaagaccag
cggctttaccatgaatcctcaggccagcctgggcgatcctttagg
catagaggactctctggaaagccaagattcaatggaattttaagt
agggcaaccacttatgag ttggtttttgcaattgagtttccctc
tgggttgcattgagggcttctcctagcaccctttactgctgtgta
tggggcttcaccatccaagaggtggtaggttggagtaagatgcta
cagatgctctcaagtcaggaatagaaactgatgagctgattgctt
gaggcttttagtgagttccgaaaagcaacaggaaaaatcagttat
ctgaaagctcagtaactcagaacaggagtaactgcaggggaccag
agatgagcaaagatctgtgtgtgttggggagctgtcatgtaaatc
aaagccaaggttgtcaaagaacagccagtgaggccaggaaagaaa
ttggtcttgtggttttcatttttttcccccttgattgattatatt
ttgtattgagatatgataagtgccttctatttcatttttgaataa
ttcttcatttttataattttacatatcttggcttgctatataaga
ttcaaaagagctttttaaatttttctaataatatcttacatttgt
acagcatgatgacctttacaaagtgctctcaatgcatttacccat
tcgttatataaatatgttacatcaggacaactttgagaaaatcag
tccttttttatgtttaaattatgtatctattgtaaccttcagagt
ttaggaggtcatctgctgtcatggatttttcaataatgaatttag
aatacacctgttagctacagttagttattaaatcttctgataata
tatgtttacttagctatcagaagccaagtatgattctttattttt
actttttcatttcaagaaatttagagtttccaaatttagagcttc
tgcatacagtcttaaagccacagaggcttgtaaaaatataggtta
gcttgatgtctaaaaatatatttcatgtcttactgaaacattttg
ccagactttctccaaatgaaacctgaatcaatttttctaaatcta
ggtttcatagagtcctctcctctgcaatgtgttattctttctata
atgatcagtttactttcagtggattcagaattgtgtagcaggata
accttgtatttttccatccgctaagtttagatggagtccaaacgc
agtacagcagaagagttaacatttacacagtgctttttaccactg
tggaatgttttcacactcatttttccttacaacaattctgaggag
taggtgttgttattatctccatttgatgggggtttaaatgatttg
ctcaaagtcatttaggggtaataaatacttggcttggaaatttaa
cacagtccttttgtctccaaagcccttcttctttccaccacaaat
taatcactatgtttataaggtagtatcagaatttttttaggattc
acaactaatcactatagcacatgaccttgggattacatttttatg
gggcaggggtaagcaagtttttaaatcatttgtgtgctctggctc
ttttgatagaagaaagcaacacaaaagctccaaagggccccctaa
ccctcttgtggctccagttatttggaaactatgatctgcatcctt
aggaatctgggatttgccagttgctggcaatgtagagcaggcatg
gaattttatatgctagtgagtcataatgatatgttagtgttaatt
agttttttcttcctttgattttattggccataattgctactcttc
atacacagtatatcaaagagcttgataatttagttgtcaaaag
Left HA
gaattcttttagagctgatgagcacaacaggagatatccagtcca
tggtcctgtggatggtaaaaccctaggccttttacgaaagaagga
aaagagagctacttcctggccggacctcattgccaaggttttccg
gatcgatgtgaaggcagatgttgactcgatccaccccactgagtt
ctgccataactgctggagcatcatgcacaggaagtttagcagtgc
accat
CORAG1 CDS
gcgaagtgtacttccccagaaacgtgaccatggaatggcaccctc
acacacccagctgcgacatctgcaacacagccagaagaggcctga
agcggaagtccctgcagcctaatctgcagctgagcaagaaactga
aaaccgtgctggaccaggccagacaggcccggcaaagaaagagaa
gggcccaagccagaatcagcagcaaggacgtgatgaagaagatcg
ccaactgcagcaagatccacctgagcaccaaactgctggccgtgg
acttccctgagcacttcgtgaagtccatcagctgccagatctgcg
agcacatcctggccgatcctgtggaaacaaactgcaagcacgtgt
tctgcagagtgtgcatcctgcggtgcctgaaagtgatgggcagct
actgcccctcctgcagatacccttgcttccccaccgatctggaaa
gccctgtgaagtccttcctgagcgtgctgaacagcctgatggtca
agtgccccgccaaagaatgcaacgaggaagtgtccctggaaaagt
acaaccaccacatcagcagccacaaagagtccaaagaaatcttcg
tgcacatcaacaaaggcggcagaccccggcagcatctgctgtctc
ttacaagacgggcccagaagcaccggctgagagaactgaagctgc
aagtgaaggcctttgccgacaaagaggaaggcggcgacgtcaaga
gcgtgtgcatgaccctgtttctgctggccctgagagcccggaatg
agcatagacaggccgatgagctggaagccatcatgcaaggcaaag
gcagcggactgcagcctgctgtgtgtctggctatcagagtgaaca
ccttcctgtcctgcagccagtaccacaagatgtaccggaccgtga
aggccattaccggcagacagatcttccagcctctgcacgccctga
gaaacgccgagaaagttctgctgcctggctaccaccacttcgagt
ggcagcctccactgaagaacgtgtccagcagcaccgacgtgggca
tcatcgatggactgagcggactgtctagcagcgtggacgactacc
ccgtggacacaatcgccaagcggttcagatacgacagcgccctgg
tgtctgccctgatggacatggaagaggacatcctggaaggcatgc
ggagccaggacctggacgattacctgaacggccctttcaccgtgg
tggtcaaagaaagctgtgacggcatgggcgacgtgtccgagaaac
acggatctggacctgtggtgccagagaaggccgtgcggttcagct
tcaccatcatgaagatcactatcgcccacagcagccagaacgtga
aagtgttcgaggaagccaagcctaacagcgagctgtgctgcaagc
ctctgtgtctgatgctggccgacgagagcgatcacgagacactga
ccgccattctgagccctctgatcgccgaacgggaagccatgaagt
cctccgagctgatgctcgaactcggcggcatcctgagaaccttca
agttcatcttccgcggcaccggctacgacgagaagctcgttagag
aggtggaaggcctggaagcctctggcagcgtgtacatctgcaccc
tgtgtgacgccaccagactggaagctagccagaacctggtgttcc
acagcatcaccagaagccacgccgaaaacctggaaagatacgaag
tgtggcggagcaacccctaccacgagagcgtggaagaactgcggg
atagagtgaagggcgtgtccgccaagcctttcatcgagacagtgc
ctagcatcgacgccctgcactgcgatattggcaacgccgccgaat
tctacaagatctttcagctggaaatcggcgaggtctacaagaacc
ccaacgcctctaaagaggaacggaagcgctggcaggccacactgg
ataagcacctgagaaagaagatgaatctgaagcccatcatgagga
tgaacggcaacttcgcccggaagctgatgaccaaagaaaccgtgg
atgccgtgtgcgagctgatcccctctgaggaaagacacgaggccc
tgcgggaactgatggacctgtacctgaagatgaagcccgtgtggc
ggtctagctgtcctgccaaagagtgccctgagtctctgtgccagt
acagcttcaacagccagagattcgccgagctgctgtccaccaagt
tcaagtacagatacgagggcaagatcaccaactacttccacaaga
ccctggctcacgtgcccgagatcatcgagagagatggctctattg
gcgcctgggcctctgagggcaatgagtctggcaacaagctgttcc
ggcggttccgcaagatgaacgccagacagagcaagtgctacgaga
tggaagatgtgctgaagcaccactggctgtacaccagcaagtacc
tgcagaaattcatgaacgcccacaacgccctcaagaccagcggct
ttaccatgaatcctcaggccagcctgggcgatccttt
Right HA
aggcatagaggactctctggaaagccaagattcaatggaatttta
agtagggcaaccacttatgagttggtttttgcaattgagtttccc
tctgggttgcattgagggcttctcctagcaccctttactgctgtg
tatggggcttcaccatccaagaggtggtaggttggagtaagatgc
tacagatgctctcaagtcaggaatagaaactgatgagctgattgc
ttgaggcttttagtgagttccgaaaagcaacaggaaaaatcagtt
atctgaaagctcagtaactcagaacaggagtaactgcaggggacc
agagatgagcaaagatctgtgtgtgttggggagctgtcatgtaaa
tcaaagccaaggttgtcaaagaacagccagtgaggccaggaaaga
aattggtcttgtggttttcatttttttcccccttgattgattata
ttttgtattgagatatgataagtgccttctatttcatttttgaat
aattcttcatttttataattttacatatcttggcttgctatataa
gattcaaaagagctttttaaatttttctaataatatcttacattt
gtacagcatgatgacctttacaaagtgctctcaatgcatttaccc
attcgttatataaatatgttacatcaggacaactttgagaaaatc
agtccttttttatgtttaaattatgtatctattgtaaccttcaga
gtttaggaggtcatctgctgtcatggatttttcaataatgaattt
agaatacacctgttagctacagttagttattaaatcttctgataa
tatatgtttacttagctatcagaagccaagtatgattctttattt
ttactttttcatttcaagaaatttagagtttccaaatttagagct
tctgcatacagtcttaaagccacagaggcttgtaaaaatataggt
tagcttgatgtctaaaaatatatttcatgtcttactgaaacattt
tgccagactttctccaaatgaaacctgaatcaatttttctaaatc
taggtttcatagagtcctctcctctgcaatgtgttattctttcta
taatgatcagtttactttcagtggattcagaattgtgtagcagga
taaccttgtatttttccatccgctaagtttagatggagtccaaac
gcagtacagcagaagagttaacatttacacagtgctttttaccac
tgtggaatgttttcacactcatttttccttacaacaattctgagg
agtaggtgttgttattatctccatttgatgggggtttaaatgatt
tgctcaaagtcatttaggggtaataaatacttggcttggaaattt
aacacagtccttttgtctccaaagcccttcttctttccaccacaa
attaatcactatgtttataaggtagtatcagaatttttttaggat
tcacaactaatcactatagcacatgaccttgggattacattttta
tggggcaggggtaagcaagtttttaaatcatttgtgtgctctggc
tcttttgatagaagaaagcaacacaaaagctccaaagggccccct
aaccctcttgtggctccagttatttggaaactatgatctgcatcc
ttaggaatctgggatttgccagttgctggcaatgtagagcaggca
tggaattttatatgctagtgagtcataatgatatgttagtgttaa
ttagttttttcttcctttgattttattggccataattgctactct
tcatacacagtatatcaaagagcttgataatttagttgtcaaaag
DONOR specific for “g8 exon2 M2/3,
“g9 exon2 M2/3” and “g12 exon2 M2/3”
gRNAs for the exon 2 RAG1 gene replacement
strategy with long right HA
INSERT
ccagtccatggtcctgtggatggtaaaaccctaggccttttacga
aagaaggaaaagagagctacttcctggccggacctcattgccaag
gttttccggatcgatgtgaaggcagatgttgactcgatccacccc
actgagttctgccataactgctggagcatcatgcacaggaagttt
agcagtgccccatgtgaggtttacttcccgaggaacgtgaccatg
gagtggcacccccacacaccatcctgtgacatctgcaacactgct
agaagaggcctgaagcggaagtccctgcagcctaatctgcagctg
agcaagaaactgaaaaccgtgctggaccaggccagacaggcccgg
caaagaaagagaagggcccaagccagaatcagcagcaaggacgtg
atgaagaagatcgccaactgcagcaagatccacctgagcaccaaa
ctgctggccgtggacttccctgagcacttcgtgaagtccatcagc
tgccagatctgcgagcacatcctggccgatcctgtggaaacaaac
tgcaagcacgtgttctgcagagtgtgcatcctgcggtgcctgaaa
gtgatgggcagctactgcccctcctgcagatacccttgcttcccc
accgatctggaaagccctgtgaagtccttcctgagcgtgctgaac
agcctgatggtcaagtgccccgccaaagaatgcaacgaggaagtg
tccctggaaaagtacaaccaccacatcagcagccacaaagagtcc
aaagaaatcttcgtgcacatcaacaaaggcggcagaccccggcag
catctgctgtctcttacaagacgggcccagaagcaccggctgaga
gaactgaagctgcaagtgaaggcctttgccgacaaagaggaaggc
ggcgacgtcaagagcgtgtgcatgaccctgtttctgctggccctg
agagcccggaatgagcatagacaggccgatgagctggaagccatc
atgcaaggcaaaggcagcggactgcagcctgctgtgtgtctggct
atcagagtgaacaccttcctgtcctgcagccagtaccacaagatg
taccggaccgtgaaggccattaccggcagacagatcttccagcct
ctgcacgccctgagaaacgccgagaaagttctgctgcctggctac
caccacttcgagtggcagcctccactgaagaacgtgtccagcagc
accgacgtgggcatcatcgatggactgagcggactgtctagcagc
gtggacgactaccccgtggacacaatcgccaagcggttcagatac
gacagcgccctggtgtctgccctgatggacatggaagaggacatc
ctggaaggcatgcggagccaggacctggacgattacctgaacggc
cctttcaccgtggtggtcaaagaaagctgtgacggcatgggcgac
gtgtccgagaaacacggatctggacctgtggtgccagagaaggcc
gtgcggttcagcttcaccatcatgaagatcactatcgcccacagc
agccagaacgtgaaagtgttcgaggaagccaagcctaacagcgag
ctgtgctgcaagcctctgtgtctgatgctggccgacgagagcgat
cacgagacactgaccgccattctgagccctctgatcgccgaacgg
gaagccatgaagtcctccgagctgatgctcgaactcggcggcatc
ctgagaaccttcaagttcatcttccgcggcaccggctacgacgag
aagctcgttagagaggtggaaggcctggaagcctctggcagcgtg
tacatctgcaccctgtgtgacgccaccagactggaagctagccag
aacctggtgttccacagcatcaccagaagccacgccgaaaacctg
gaaagatacgaagtgtggcggagcaacccctaccacgagagcgtg
gaagaactgcgggatagagtgaagggcgtgtccgccaagcctttc
atcgagacagtgcctagcatcgacgccctgcactgcgatattggc
aacgccgccgaattctacaagatctttcagctggaaatcggcgag
gtctacaagaaccccaacgcctctaaagaggaacggaagcgctgg
caggccacactggataagcacctgagaaagaagatgaatctgaag
cccatcatgaggatgaacggcaacttcgcccggaagctgatgacc
aaagaaaccgtggatgccgtgtgcgagctgatcccctctgaggaa
agacacgaggccctgcgggaactgatggacctgtacctgaagatg
aagcccgtgtggcggtctagctgtcctgccaaagagtgccctgag
tctctgtgccagtacagcttcaacagccagagattcgccgagctg
ctgtccaccaagttcaagtacagatacgagggcaagatcaccaac
tacttccacaagaccctggctcacgtgcccgagatcatcgagaga
gatggctctattggcgcctgggcctctgagggcaatgagtctggc
aacaagctgttccggcggttccgcaagatgaacgccagacagagc
aagtgctacgagatggaagatgtgctgaagcaccactggctgtac
accagcaagtacctgcagaaattcatgaacgcccacaacgccctc
aagaccagcggctttaccatgaatcctcaggccagcctgggcgat
cctttaggcatagaggactctctggaaagccaagattcaatggaa
ttttaagtagggcaaccacttatgagttggtttttgcaattgagt
ttccctctgggttgcattgagggcttctcctagcaccctttactg
ctgtgtatggggcttcaccatccaagaggtggtaggttggagtaa
gatgctacagatgctctcaagtcaggaatagaaactgatgagctg
attgcttgaggcttttagtgagttccgaaaagcaacaggaaaaat
cagttatctgaaagctcagtaactcagaacaggagtaactgcagg
ggaccagagatgagcaaagatctgtgtgtgttggggagctgtcat
gtaaatcaaagccaaggttgtcaaagaacagccagtgaggccagg
aaagaaattggtcttgtggttttcatttttttcccccttgattga
ttatattttgtattgagatatgataagtgccttctatttcatttt
tgaataattcttcatttttataattttacatatcttggcttgcta
tataagattcaaaagagctttttaaatttttctaataatatctta
catttgtacagcatgatgacctttacaaagtgctctcaatgcatt
tacccattcgttatataaatatgttacatcaggacaactttgaga
aaatcagtccttttttatgtttaaattatgtatctattgtaacct
tcagagtttaggaggtcatctgctgtcatggatttttcaataatg
aatttagaatacacctgttagctacagttagttattaaatcttct
gataatatatgtttacttagctatcagaagccaagtatgattctt
tatttttactttttcatttcaagaaatttagagtttccaaattta
gagcttctgcatacagtcttaaagccacagaggcttgtaaaaata
taggttagcttgatgtctaaaaatatatttcatgtcttactgaaa
cattttgccagactttctccaaatgaaacctgaatcaatttttct
aaatctaggtttcatagagtcctctcctctgcaatgtgttattct
ttctataatgatcagtttactttcagtggattcagaattgtgtag
caggataaccttgtatttttccatccgctaagtttagatggagtc
caaacgcagtacagcagaagagttaacatttacacagtgcttttt
accactgtggaatgttttcacactcatttttccttacaacaattc
tgaggagtaggtgttgttattatctccatttgatgggggtttaaa
tgatttgctcaaagtcatttaggggtaataaatacttggcttgga
aatttaacacagtccttttgtctccaaagcccttcttctttccac
cacaaattaatcactatgtttataaggtagtatcagaattttttt
aggattcacaactaatcactatagcacatgaccttgggattacat
ttttatggggcaggggtaagcaagtttttaaatcatttgtgtgct
ctggctcttttgatagaagaaagcaacacaaaagctccaaagggc
cccctaaccctcttgtggctccagttatttggaaactatgatctg
catccttaggaatctgggatttgccagttgctggcaatgtagagc
aggcatggaattttatatgctagtgagtcataatgatatgttagt
gttaattagttttttcttcctttgattttattggccataattgct
actcttcatacacagtatatcaaagagcttgataatttagttgtc
aaaag
Left HA
ccagtccatggtcctgtggatggtaaaaccctaggccttttacga
aagaaggaaaagagagctacttcctggccggacctcattgccaag
gttttccggatcgatgtgaaggcagatgttgactcgatccacccc
actgagttctgccataactgctggagcatcatgcacaggaagttt
agcagtgccccatgtgaggtttacttcccgaggaacgtgaccatg
gagtggcacccccacacaccatcctgtgacatctgcaacactgc
coRAG1 CDS
tagaagaggcctgaagcggaagtccctgcagcctaatctgcagct
gagcaagaaactgaaaaccgtgctggaccaggccagacaggcccg
gcaaagaaagagaagggcccaagccagaatcagcagcaaggacgt
gatgaagaagatcgccaactgcagcaagatccacctgagcaccaa
actgctggccgtggacttccctgagcacttcgtgaagtccatcag
ctgccagatctgcgagcacatcctggccgatcctgtggaaacaaa
ctgcaagcacgtgttctgcagagtgtgcatcctgcggtgcctgaa
agtgatgggcagctactgcccctcctgcagatacccttgcttccc
caccgatctggaaagccctgtgaagtccttcctgagcgtgctgaa
cagcctgatggtcaagtgccccgccaaagaatgcaacgaggaagt
gtccctggaaaagtacaaccaccacatcagcagccacaaagagtc
caaagaaatcttcgtgcacatcaacaaaggcggcagaccccggca
gcatctgctgtctcttacaagacgggcccagaagcaccggctgag
agaactgaagctgcaagtgaaggcctttgccgacaaagaggaagg
cggcgacgtcaagagcgtgtgcatgaccctgtttctgctggccct
gagagcccggaatgagcatagacaggccgatgagctggaagccat
catgcaaggcaaaggcagcggactgcagcctgctgtgtgtctggc
tatcagagtgaacaccttcctgtcctgcagccagtaccacaagat
gtaccggaccgtgaaggccattaccggcagacagatcttccagcc
tctgcacgccctgagaaacgccgagaaagttctgctgcctggcta
ccaccacttcgagtggcagcctccactgaagaacgtgtccagcag
caccgacgtgggcatcatcgatggactgagcggactgtctagcag
cgtggacgactaccccgtggacacaatcgccaagcggttcagata
cgacagcgccctggtgtctgccctgatggacatggaagaggacat
cctggaaggcatgcggagccaggacctggacgattacctgaacgg
ccctttcaccgtggtggtcaaagaaagctgtgacggcatgggcga
cgtgtccgagaaacacggatctggacctgtggtgccagagaaggc
cgtgcggttcagcttcaccatcatgaagatcactatcgcccacag
cagccagaacgtgaaagtgttcgaggaagccaagcctaacagcga
gctgtgctgcaagcctctgtgtctgatgctggccgacgagagcga
tcacgagacactgaccgccattctgagccctctgatcgccgaacg
ggaagccatgaagtcctccgagctgatgctcgaactcggcggcat
cctgagaaccttcaagttcatcttccgcggcaccggctacgacga
gaagctcgttagagaggtggaaggcctggaagcctctggcagcgt
gtacatctgcaccctgtgtgacgccaccagactggaagctagcca
gaacctggtgttccacagcatcaccagaagccacgccgaaaacct
ggaaagatacgaagtgtggcggagcaacccctaccacgagagcgt
ggaagaactgcgggatagagtgaagggcgtgtccgccaagccttt
catcgagacagtgcctagcatcgacgccctgcactgcgatattgg
caacgccgccgaattctacaagatctttcagctggaaatcggcga
ggtctacaagaaccccaacgcctctaaagaggaacggaagcgctg
gcaggccacactggataagcacctgagaaagaagatgaatctgaa
gcccatcatgaggatgaacggcaacttcgcccggaagctgatgac
caaagaaaccgtggatgccgtgtgcgagctgatcccctctgagga
aagacacgaggccctgcgggaactgatggacctgtacctgaagat
gaagcccgtgtggcggtctagctgtcctgccaaagagtgccctga
gtctctgtgccagtacagcttcaacagccagagattcgccgagct
gctgtccaccaagttcaagtacagatacgagggcaagatcaccaa
ctacttccacaagaccctggctcacgtgcccgagatcatcgagag
agatggctctattggcgcctgggcctctgagggcaatgagtctgg
caacaagctgttccggcggttccgcaagatgaacgccagacagag
caagtgctacgagatggaagatgtgctgaagcaccactggctgta
caccagcaagtacctgcagaaattcatgaacgcccacaacgccct
caagaccagcggctttaccatgaatcctcaggccagcctgggcga
tccttt
Right HA
aggcatagaggactctctggaaagccaagattcaatggaatttta
agtagggcaaccacttatgagttggtttttgcaattgagtttccc
tctgggttgcattgagggcttctcctagcaccctttactgctgtg
tatggggcttcaccatccaagaggtggtaggttggagtaagatgc
tacagatgctctcaagtcaggaatagaaactgatgagctgattgc
ttgaggcttttagtgagttccgaaaagcaacaggaaaaatcagtt
atctgaaagctcagtaactcagaacaggagtaactgcaggggacc
agagatgagcaaagatctgtgtgtgttggggagctgtcatgtaaa
tcaaagccaaggttgtcaaagaacagccagtgaggccaggaaaga
aattggtcttgtggttttcatttttttcccccttgattgattata
ttttgtattgagatatgataagtgccttctatttcatttttgaat
aattcttcatttttataattttacatatcttggcttgctatataa
gattcaaaagagctttttaaatttttctaataatatcttacattt
gtacagcatgatgacctttacaaagtgctctcaatgcatttaccc
attcgttatataaatatgttacatcaggacaactttgagaaaatc
agtccttttttatgtttaaattatgtatctattgtaaccttcaga
gtttaggaggtcatctgctgtcatggatttttcaataatgaattt
agaatacacctgttagctacagttagttattaaatcttctgataa
tatatgtttacttagctatcagaagccaagtatgattctttattt
ttactttttcatttcaagaaatttagagtttccaaatttagagct
tctgcatacagtcttaaagccacagaggcttgtaaaaatataggt
tagcttgatgtctaaaaatatatttcatgtcttactgaaacattt
tgccagactttctccaaatgaaacctgaatcaatttttctaaatc
taggtttcatagagtcctctcctctgcaatgtgttattctttcta
taatgatcagtttactttcagtggattcagaattgtgtagcagga
taaccttgtatttttccatccgctaagtttagatggagtccaaac
gcagtacagcagaagagttaacatttacacagtgctttttaccac
tgtggaatgttttcacactcatttttccttacaacaattctgagg
agtaggtgttgttattatctccatttgatgggggtttaaatgatt
tgctcaaagtcatttaggggtaataaatacttggcttggaaattt
aacacagtccttttgtctccaaagcccttcttctttccaccacaa
attaatcactatgtttataaggtagtatcagaatttttttaggat
tcacaactaatcactatagcacatgaccttgggattacattttta
tggggcaggggtaagcaagtttttaaatcatttgtgtgctctggc
tcttttgatagaagaaagcaacacaaaagctccaaagggccccct
aaccctcttgtggctccagttatttggaaactatgatctgcatcc
ttaggaatctgggatttgccagttgctggcaatgtagagcaggca
tggaattttatatgctagtgagtcataatgatatgttagtgttaa
ttagttttttcttcctttgattttattggccataattgctactct
tcatacacagtatatcaaagagcttgataatttagttgtcaaaag
DONOR specific for “g11 exon2 M2/3”
gRNA for the exon 2 RAG1 gene replacement
strategy with long right HA
INSERT
ctgatgagcacaacaggagatatccagtccatggtcctgtggatg
gtaaaaccctaggccttttacgaaagaaggaaaagagagctactt
cctggccggacctcattgccaaggttttccggatcgatgtgaagg
cagatgttgactcgatccaccccactgagttctgccataactgct
ggagcatcatgcacaggaagtttagcagtgccccatgtgaggttt
acttccccagaaacgtgaccatggaatggcaccctcacacaccca
gctgcgacatctgcaacacagccagaagaggcctgaagcggaagt
ccctgcagcctaatctgcagctgagcaagaaactgaaaaccgtgc
tggaccaggccagacaggcccggcaaagaaagagaagggcccaag
ccagaatcagcagcaaggacgtgatgaagaagatcgccaactgca
gcaagatccacctgagcaccaaactgctggccgtggacttccctg
agcacttcgtgaagtccatcagctgccagatctgcgagcacatcc
tggccgatcctgtggaaacaaactgcaagcacgtgttctgcagag
tgtgcatcctgcggtgcctgaaagtgatgggcagctactgcccct
cctgcagatacccttg cttccccaccgatctggaaagccctgtg
aagtccttcctgagcgtgctgaacagcctgatggtcaagtgcccc
gccaaagaatgcaacgaggaagtgtccctggaaaagtacaaccac
cacatcagcagccacaaagagtccaaagaaatcttcgtgcacatc
aacaaaggcggcagaccccggcagcatctgctgtctcttacaaga
cgggcccagaagcaccggctgagagaactgaagctgcaagtgaag
gcctttgccgacaaagaggaaggcggcgacgtcaagagcgtgtgc
atgaccctgtttctgctggccctgagagcccggaatgagcataga
caggccgatgagctggaagccatcatgcaaggcaaaggcagcgga
ctgcagcctgctgtgtgtctggctatcagagtgaacaccttcctg
tcctgcagccagtaccacaagatgtaccggaccgtgaaggccatt
accggcagacagatcttccagcctctgcacgccctgagaaacgcc
gagaaagttctgctgcctggctaccaccacttcgagtggcagcct
ccactgaagaacgtgtccagcagcaccgacgtgggcatcatcgat
ggactgagcggactgtctagcagcgtggacgactaccccgtggac
acaatcgccaagcggttcagatacgacagcgccctggtgtctgcc
ctgatggacatggaagaggacatcctggaaggcatgcggagccag
gacctggacgattacctgaacggccctttcaccgtggtggtcaaa
gaaagctgtgacggcatgggcgacgtgtccgagaaacacggatct
ggacctgtggtgccagagaaggccgtgcggttcagcttcaccatc
atgaagatcactatcgcccacagcagccagaacgtgaaagtgttc
gaggaagccaagcctaacagcgagctgtgctgcaagcctctgtgt
ctgatgctggccgacgagagcgatcacgagacactgaccgccatt
ctgagccctctgatcgccgaacgggaagccatgaagtcctccgag
ctgatgctcgaactcggcggcatcctgagaaccttcaagttcatc
ttccgcggcaccggctacgacgagaagctcgttagagaggtggaa
ggcctggaagcctctggcagcgtgtacatctgcaccctgtgtgac
gccaccagactggaagctagccagaacctggtgttccacagcatc
accagaagccacgccgaaaacctggaaagatacgaagtgtggcgg
agcaacccctaccacgagagcgtggaagaactgcgggatagagtg
aagggcgtgtccgccaagcctttcatcgagacagtgcctagcatc
gacgccctgcactgcgatattggcaacgccgccgaattctacaag
atctttcagctggaaatcggcgaggtctacaagaaccccaacgcc
tctaaagaggaacggaagcgctggcaggccacactggataagcac
ctgagaaagaagatgaatctgaagcccatcatgaggatgaacggc
aacttcgcccggaagctgatgaccaaagaaaccgtggatgccgtg
tgcgagctgatcccctctgaggaaagacacgaggccctgcgggaa
ctgatggacctgtacctgaagatgaagcccgtgtggcggtctagc
tgtcctgccaaagagtgccctgagtctctgtgccagtacagcttc
aacagccagagattcgccgagctgctgtccaccaagttcaagtac
agatacgagggcaagatcaccaactacttccacaagaccctggct
cacgtgcccgagatcatcgagagagatggctctattggcgcctgg
gcctctgagggcaatgagtctggcaacaagctgttccggcggttc
cgcaagatgaacgccagacagagcaagtgctacgagatggaagat
gtgctgaagcaccactggctgtacaccagcaagtacctgcagaaa
ttcatgaacgcccacaacgccctcaagaccagcggctttaccatg
aatcctcaggccagcctgggcgatcctttaggcatagaggactct
ctggaaagccaagattcaatggaattttaagtagggcaaccactt
atgagttggtttttgcaattgagtttccctctgggttgcattgag
ggcttctcctagcaccctttactgctgtgtatggggcttcaccat
ccaagaggtggtaggttggagtaagatgctacagatgctctcaag
tcaggaatagaaactgatgagctgattgcttgaggcttttagtga
gttccgaaaagcaacaggaaaaatcagttatctgaaagctcagta
actcagaacaggagtaactgcaggggaccagagatgagcaaagat
ctgtgtgtgttggggagctgtcatgtaaatcaaagccaaggttgt
caaagaacagccagtgaggccaggaaagaaattggtcttgtggtt
ttcatttttttcccccttgattgattatattttgtattgagatat
gataagtgccttctatttcatttttgaataattcttcatttttat
aattttacatatcttggcttgctatataagattcaaaagagcttt
ttaaatttttctaataatatcttacatttgtacagcatgatgacc
tttacaaagtgctctcaatgcatttacccattcgttatataaata
tgttacatcaggacaactttgagaaaatcagtccttttttatgtt
taaattatgtatctattgtaaccttcagagtttaggaggtcatct
gctgtcatggatttttcaataatgaatttagaatacacctgttag
ctacagttagttattaaatcttctgataatatatgtttacttagc
tatcagaagccaagtatgattctttatttttactttttcatttca
agaaatttagagtttccaaatttagagcttctgcatacagtctta
aagccacagaggcttgtaaaaatataggttagcttgatgtctaaa
aatatatttcatgtcttactgaaacattttgccagactttctcca
aatgaaacctgaatcaatttttctaaatctaggtttcatagagtc
ctctcctctgcaatgtgttattctttctataatgatcagtttact
ttcagtggattcagaattgtgtagcaggataaccttgtatttttc
catccgctaagtttagatggagtccaaacgcagtacagcagaaga
gttaacatttacacagtgctttttaccactgtggaatgttttcac
actcatttttccttacaacaattctgaggagtaggtgttgttatt
atctccatttgatgggggtttaaatgatttgctcaaagtcattta
ggggtaataaatacttggcttggaaatttaacacagtccttttgt
ctccaaagcccttcttctttccaccacaaattaatcactatgttt
ataaggtagtatcagaatttttttaggattcacaactaatcacta
tagcacatgaccttgggattacatttttatggggcaggggtaagc
aagtttttaaatcatttgtgtgctctggctcttttgatagaagaa
agcaacacaaaagctccaaagggccccctaaccctcttgtggctc
cagttatttggaaactatgatctgcatccttaggaatctgggatt
tgccagttgctggcaatgtagagcaggcatggaattttatatgct
agtgagtcataatgatatgttagtgttaattagttttttcttcct
ttgattttattggccataattgctactcttcatacacagtatatc
aaagagcttgataatttagttgtcaaaag
Left HA
ctgatgagcacaacaggagatatccagtccatggtcctgtggatg
gtaaaaccctaggccttttacgaaagaaggaaaagagagctactt
cctggccggacctcattgccaaggttttccggatcgatgtgaagg
cagatgttgactcgatccaccccactgagttctgccataactgct
ggagcatcatgcacaggaagtttagcagtgccccatgtgaggttt
acttc
coRAG1 CDS
cccagaaacgtgaccatggaatggcaccctcacacacccagctgc
gacatctgcaacacagccagaagaggcctgaagcggaagtccctg
cagcctaatctgcagctgagcaagaaactgaaaaccgtgctggac
caggccagacaggcccggcaaagaaagagaagggcccaagccaga
atcagcagcaaggacgtgatgaagaagatcgccaactgcagcaag
atccacctgagcaccaaactgctggccgtggacttccctgagcac
ttcgtgaagtccatcagctgccagatctgcgagcacatcctggcc
gatcctgtggaaacaaactgcaagcacgtgttctgcagagtgtgc
atcctgcggtgcctgaaagtgatgggcagctactgcccctcctgc
agatacccttgcttccccaccgatctggaaagccctgtgaagtcc
ttcctgagcgtgctgaacagcctgatggtcaagtgccccgccaaa
gaatgcaacgaggaagtgtccctggaaaagtacaaccaccacatc
agcagccacaaagagtccaaagaaatcttcgtgcacatcaacaaa
ggcggcagaccccggcagcatctgctgtctcttacaagacgggcc
cagaagcaccggctgagagaactgaagctgcaagtgaaggccttt
gccgacaaagaggaaggcggcgacgtcaagagcgtgtgcatgacc
ctgtttctgctggccctgagagcccggaatgagcatagacaggcc
gatgagctggaagccatcatgcaaggcaaaggcagcggactgcag
cctgctgtgtgtctggctatcagagtgaacaccttcctgtcctgc
agccagtaccacaagatgtaccggaccgtgaaggccattaccggc
agacagatcttccagcctctgcacgccctgagaaacgccgagaaa
gttctgctgcctggctaccaccacttcgagtggcagcctccactg
aagaacgtgtccagcagcaccgacgtgggcatcatcgatggactg
agcggactgtctagcagcgtggacgactaccccgtggacacaatc
gccaagcggttcagatacgacagcgccctggtgtctgccctgatg
gacatggaagaggacatcctggaaggcatgcggagccaggacctg
gacgattacctgaacggccctttcaccgtggtggtcaaagaaagc
tgtgacggcatgggcgacgtgtccgagaaacacggatctggacct
gtggtgccagagaaggccgtgcggttcagcttcaccatcatgaag
atcactatcgcccacagcagccagaacgtgaaagtgttcgaggaa
gccaagcctaacagcgagctgtgctgcaagcctctgtgtctgatg
ctggccgacgagagcgatcacgagacactgaccgccattctgagc
cctctgatcgccgaacgggaagccatgaagtcctccgagctgatg
ctcgaactcggcggcatcctgagaaccttcaagttcatcttccgc
ggcaccggctacgacgagaagctcgttagagaggtggaaggcctg
gaagcctctggcagcgtgtacatctgcaccctgtgtgacgccacc
agactggaagctagccagaacctggtgttccacagcatcaccaga
agccacgccgaaaacctggaaagatacgaagtgtggcggagcaac
ccctaccacgagagcgtggaagaactgcgggatagagtgaagggc
gtgtccgccaagcctttcatcgagacagtgcctagcatcgacgcc
ctgcactgcgatattggcaacgccgccgaattctacaagatcttt
cagctggaaatcggcgaggtctacaagaaccccaacgcctctaaa
gaggaacggaagcgctggcaggccacactggataagcacctgaga
aagaagatgaatctgaagcccatcatgaggatgaacggcaacttc
gcccggaagctgatgaccaaagaaaccgtggatgccgtgtgcgag
ctgatcccctctgaggaaagacacgaggccctgcgggaactgatg
gacctgtacctgaagatgaagcccgtgtggcggtctagctgtcct
gccaaagagtgccctgagtctctgtgccagtacagcttcaacagc
cagagattcgccgagctgctgtccaccaagttcaagtacagatac
gagggcaagatcaccaactacttccacaagaccctggctcacgtg
cccgagatcatcgagagagatggctctattggcgcctgggcctct
gagggcaatgagtctggcaacaagctgttccggcggttccgcaag
atgaacgccagacagagcaagtgctacgagatggaagatgtgctg
aagcaccactggctgtacaccagcaagtacctgcagaaattcatg
aacgcccacaacgccctcaagaccagcggctttaccatgaatcct
caggccagcctgggcgatccttt
Right HA
aggcatagaggactctctggaaagccaagattcaatggaatttta
agtagggcaaccacttatgagttggtttttgcaattgagtttccc
tctgggttgcattgagggcttctcctagcaccctttactgctgtg
tatggggcttcaccatccaagaggtggtaggttggagtaagatgc
tacagatgctctcaagtcaggaatagaaactgatgagctgattgc
ttgaggcttttagtgagttccgaaaagcaacaggaaaaatcagtt
atctgaaagctcagtaactcagaacaggagtaactgcaggggacc
agagatgagcaaagatctgtgtgtgttggggagctgtcatgtaaa
tcaaagccaaggttgtcaaagaacagccagtgaggccaggaaaga
aattggtcttgtggttttcatttttttcccccttgattgattata
ttttgtattgagatatgataagtgccttctatttcatttttgaat
aattcttcatttttataattttacatatcttggcttgctatataa
gattcaaaagagctttttaaatttttctaataatatcttacattt
gtacagcatgatgacctttacaaagtgctctcaatgcatttaccc
attcgttatataaatatgttacatcaggacaactttgagaaaatc
agtccttttttatgtttaaattatgtatctattgtaaccttcaga
gtttaggaggtcatctgctgtcatggatttttcaataatgaattt
agaatacacctgttagctacagttagttattaaatcttctgataa
tatatgtttacttagctatcagaagccaagtatgattctttattt
ttactttttcatttcaagaaatttagagtttccaaatttagagct
tctgcatacagtcttaaagccacagaggcttgtaaaaatataggt
tagcttgatgtctaaaaatatatttcatgtcttactgaaacattt
tgccagactttctccaaatgaaacctgaatcaatttttctaaatc
taggtttcatagagtcctctcctctgcaatgtgttattctttcta
taatgatcagtttactttcagtggattcagaattgtgtagcagga
taaccttgtatttttccatccgctaagtttagatggagtccaaac
gcagtacagcagaagagttaacatttacacagtgctttttaccac
tgtggaatgttttcacactcatttttccttacaacaattctgagg
agtaggtgttgttattatctccatttgatgggggtttaaatgatt
tgctcaaagtcatttaggggtaataaatacttggcttggaaattt
aacacagtccttttgtctccaaagcccttcttctttccaccacaa
attaatcactatgtttataaggtagtatcagaatttttttaggat
tcacaactaatcactatagcacatgaccttgggattacattttta
tggggcaggggtaagcaagtttttaaatcatttgtgtgctctggc
tcttttgatagaagaaagcaacacaaaagctccaaagggccccct
aaccctcttgtggctccagttatttggaaactatgatctgcatcc
ttaggaatctgggatttgccagttgctggcaatgtagagcaggca
tggaattttatatgctagtgagtcataatgatatgttagtgttaa
ttagttttttcttcctttgattttattggccataattgctactct
tcatacacagtatatcaaagagcttgataatttagttgtcaaaag
DONOR specific for “g14 exon2 M5”
gRNA for the exon 2 RAG1 gene replacement
strategy with long right HA
INSERT
catggagtggcacccccacacaccatcctgtgacatctgcaacac
tgcccgtcggggactcaagaggaagagtcttcagccaaacttgca
gctcagcaaaaaactcaaaactgtgcttgaccaagcaagacaagc
ccgtcagcgcaagagaagagctcaggcaaggatcagcagcaagga
tgtcatgaagaagatcgccaactgcagtaagatacatcttagtac
caagctccttgcagtggacttcccagagcactttgtgaaatccat
ctcctgccagatctgtgaacacattctggctgaccctgtggagac
caactgtaagcatgtcttttgccgggtctgcattctcagatgcct
caaagtcatgggcagctattgtccctcttgccgatatccatgctt
ccctactgacctggagagtccagtgaagtcctttctgagcgtctt
gaattccctgatggtgaaatgtccagcaaaagagtgcaatgagga
ggtcaccctagaaaagtacaaccaccacatcagcagccacaaaga
gtccaaagaaatcttcgtgcacatcaacaaaggcggcagaccccg
gcagcatctgctgtctcttacaagacgggcccagaagcaccggct
gagagaactgaagctgcaagtgaaggcctttgccgacaaagagga
aggcggcgacgtcaagagcgtgtgcatgaccctgtttctgctggc
cctgagagcccggaatgagcatagacaggccgatgagctggaagc
catcatgcaaggcaaaggcagcggactgcagcctgctgtgtgtct
ggctatcagagtgaacaccttcctgtcctgcagccagtaccacaa
gatgtaccggaccgtgaaggccattaccggcagacagatcttcca
gcctctgcacgccctgagaaacgccgagaaagttctgctgcctgg
ctaccaccacttcgagtggcagcctccactgaagaacgtgtccag
cagcaccgacgtgggcatcatcgatggactgagcggactgtctag
cagcgtggacgactaccccgtggacacaatcgccaagcggttcag
atacgacagcgccctggtgtctgccctgatggacatggaagagga
catcctggaaggcatgcggagccaggacctggacgattacctgaa
cggccctttcaccgtggtggtcaaagaaagctgtgacggcatggg
cgacgtgtccgagaaacacggatctggacctgtggtgccagagaa
ggccgtgcggttcagcttcaccatcatgaagatcactatcgccca
cagcagccagaacgtgaaagtgttcgaggaagccaagcctaacag
cgagctgtgctgcaagcctctgtgtctgatgctggccgacgagag
cgatcacgagacactgaccgccattctgagccctctgatcgccga
acgggaagccatgaagtcctccgagctgatgctcgaactcggcgg
catcctgagaaccttcaagttcatcttccgcggcaccggctacga
cgagaagctcgttagagaggtggaaggcctggaagcctctggcag
cgtgtacatctgcaccctgtgtgacgccaccagactggaagctag
ccagaacctggtgttccacagcatcaccagaagccacgccgaaaa
cctggaaagatacgaagtgtggcggagcaacccctaccacgagag
cgtggaagaactgcgggatagagtgaagggcgtgtccgccaagcc
tttcatcgagacagtgcctagcatcgacgccctgcactgcgatat
tggcaacgccgccgaattctacaagatctttcagctggaaatcgg
cgaggtctacaagaaccccaacgcctctaaagaggaacggaagcg
ctggcaggccacactggataagcacctgagaaagaagatgaatct
gaagcccatcatgaggatgaacggcaacttcgcccggaagctgat
gaccaaagaaaccgtggatgccgtgtgcgagctgatcccctctga
ggaaagacacgaggccctgcgggaactgatggacctgtacctgaa
gatgaagcccgtgtggcggtctagctgtcctgccaaagagtgccc
tgagtctctgtgccagtacagcttcaacagccagagattcgccga
gctgctgtccaccaagttcaagtacagatacgagggcaagatcac
caactacttccacaagaccctggctcacgtgcccgagatcatcga
gagagatggctctattggcgcctgggcctctgagggcaatgagtc
tggcaacaagctgttccggcggttccgcaagatgaacgccagaca
gagcaagtgctacgagatggaagatgtgctgaagcaccactggct
gtacaccagcaagtacctgcagaaattcatgaacgcccacaacgc
cctcaagaccagcggctttaccatgaatcctcaggccagcctggg
cgatcctttaggcatagaggactctctggaaagccaagattcaat
ggaattttaagtagggcaaccacttatgagttggtttttgcaatt
gagtttccctctgggttgcattgagggcttctcctagcacccttt
actgctgtgtatggggcttcaccatccaagaggtggtaggttgga
gtaagatgctacagatgctctcaagtcaggaatagaaactgatga
gctgattgcttgaggcttttagtgagttccgaaaagcaacaggaa
aaatcagttatctgaaagctcagtaactcagaacaggagtaactg
caggggaccagagatgagcaaagatctgtgtgtgttggggagctg
tcatgtaaatcaaagccaaggttgtcaaagaacagccagtgaggc
caggaaagaaattggtcttgtggttttcatttttttcccccttga
ttgattatattttgtattgagatatgataagtgccttctatttca
tttttgaataattcttcatttttataattttacatatcttggctt
gctatataagattcaaaagagctttttaaatttttctaataatat
cttacatttgtacagcatgatgacctttacaaagtgctctcaatg
catttacccattcgttatataaatatgttacatcaggacaacttt
gagaaaatcagtccttttttatgtttaaattatgtatctattgta
accttcagagtttaggaggtcatctgctgtcatggatttttcaat
aatgaatttagaatacacctgttagctacagttagttattaaatc
ttctgataatatatgtttacttagctatcagaagccaagtatgat
tctttatttttactttttcatttcaagaaatttagagtttccaaa
tttagagcttctgcatacagtcttaaagccacagaggcttgtaaa
aatataggttagcttgatgtctaaaaatatatttcatgtcttact
gaaacattttgccagactttctccaaatgaaacctgaatcaattt
ttctaaatctaggtttcatagagtcctctcctctgcaatgtgtta
ttctttctataatgatcagtttactttcagtggattcagaattgt
gtagcaggataaccttgtatttttccatccgctaagtttagatgg
agtccaaacgcagtacagcagaagagttaacatttacacagtgct
ttttaccactgtggaatgttttcacactcatttttccttacaaca
attctgaggagtaggtgttgttattatctccatttgatgggggtt
taaatgatttgctcaaagtcatttaggggtaataaatacttggct
tggaaatttaacacagtccttttgtctccaaagcccttcttcttt
ccaccacaaattaatcactatgtttataaggtagtatcagaattt
ttttaggattcacaactaatcactatagcacatgaccttgggatt
acatttttatggggcaggggtaagcaagtttttaaatcatttgtg
tgctctggctcttttgatagaagaaagcaacacaaaagctccaaa
gggccccctaaccctcttgtggctccagttatttggaaactatga
tctgcatccttaggaatctgggatttgccagttgctggcaatgta
gagcaggcatggaattttatatgctagtgagtcataatgatatgt
tagtgttaattagttttttcttcctttgattttattggccataat
tgctactcttcatacacagtatatcaaagagcttgataatttagt
tgtcaaaag
Left HA
catggagtggcacccccacacaccatcctgtgacatctgcaacac
tgcccgtcggggactcaagaggaagagtcttcagccaaacttgca
gctcagcaaaaaactcaaaactgtgcttgaccaagcaagacaagc
ccgtcagcgcaagagaagagctcaggcaaggatcagcagcaagga
tgtcatgaagaagatcgccaactgcagtaagatacatcttagtac
caagctccttgcagtggacttcccagagcactttgtgaaatccat
ctcctgccagatctgtgaacacattctggctgaccctgtggagac
caactgtaagcatgtcttttgccgggtctgcattctcagatgcct
caaagtcatgggcagctattgtccctcttgccgatatccatgctt
ccctactgacctggagagtccagtgaagtcctttctgagcgtctt
gaattccctgatggtgaaatgtccagcaaaagagtgcaatgagga
ggtca
coRAG1 CDS
ccctagaaaagtacaaccaccacatcagcagccacaaagagtcca
aagaaatcttcgtgcacatcaacaaaggcggcagaccccggcagc
atctgctgtctcttacaagacgggcccagaagcaccggctgagag
aactgaagctgcaagtgaaggcctttgccgacaaagaggaaggcg
gcgacgtcaagagcgtgtgcatgaccctgtttctgctggccctga
gagcccggaatgagcatagacaggccgatgagctggaagccatca
tgcaaggcaaaggcagcggactgcagcctgctgtgtgtctggcta
tcagagtgaacaccttcctgtcctgcagccagtaccacaagatgt
accggaccgtgaaggccattaccggcagacagatcttccagcctc
tgcacgccctgagaaacgccgagaaagttctgctgcctggctacc
accacttcgagtggcagcctccactgaagaacgtgtccagcagca
ccgacgtgggcatcatcgatggactgagcggactgtctagcagcg
tggacgactaccccgtggacacaatcgccaagcggttcagatacg
acagcgccctggtgtctgccctgatggacatggaagaggacatcc
tggaaggcatgcggagccaggacctggacgattacctgaacggcc
ctttcaccgtggtggtcaaagaaagctgtgacggcatgggcgacg
tgtccgagaaacacggatctggacctgtggtgccagagaaggccg
tgcggttcagcttcaccatcatgaagatcactatcgcccacagca
gccagaacgtgaaagtgttcgaggaagccaagcctaacagcgagc
tgtgctgcaagcctctgtgtctgatgctggccgacgagagcgatc
acgagacactgaccgccattctgagccctctgatcgccgaacggg
aagccatgaagtcctccgagctgatgctcgaactcggcggcatcc
tgagaaccttcaagttcatcttccgcggcaccggctacgacgaga
agctcgttagagaggtggaaggcctggaagcctctggcagcgtgt
acatctgcaccctgtgtgacgccaccagactggaagctagccaga
acctggtgttccacagcatcaccagaagccacgccgaaaacctgg
aaagatacgaagtgtggcggagcaacccctaccacgagagcgtgg
aagaactgcgggatagagtgaagggcgtgtccgccaagcctttca
tcgagacagtgcctagcatcgacgccctgcactgcgatattggca
acgccgccgaattctacaagatctttcagctggaaatcggcgagg
tctacaagaaccccaacgcctctaaagaggaacggaagcgctggc
aggccacactggataagcacctgagaaagaagatgaatctgaagc
ccatcatgaggatgaacggcaacttcgcccggaagctgatgacca
aagaaaccgtggatgccgtgtgcgagctgatcccctctgaggaaa
gacacgaggccctgcgggaactgatggacctgtacctgaagatga
agcccgtgtggcggtctagctgtcctgccaaagagtgccctgagt
ctctgtgccagtacagcttcaacagccagagattcgccgagctgc
tgtccaccaagttcaagtacagatacgagggcaagatcaccaact
acttccacaagaccctggctcacgtgcccgagatcatcgagagag
atggctctattggcgcctgggcctctgagggcaatgagtctggca
acaagctgttccggcggttccgcaagatgaacgccagacagagca
agtgctacgagatggaagatgtgctgaagcaccactggctgtaca
ccagcaagtacctgcagaaattcatgaacgcccacaacgccctca
agaccagcggctttaccatgaatcctcaggccagcctgggcgatc
cttt
Right HA
aggcatagaggactctctggaaagccaagattcaatggaatttta
agtagggcaaccacttatgagttggtttttgcaattgagtttccc
tctgggttgcattgagggcttctcctagcaccctttactgctgtg
tatggggcttcaccatccaagaggtggtaggttggagtaagatgc
tacagatgctctcaagtcaggaatagaaactgatgagctgattgc
ttgaggcttttagtgagttccgaaaagcaacaggaaaaatcagtt
atctgaaagctcagtaactcagaacaggagtaactgcaggggacc
agagatgagcaaagatctgtgtgtgttggggagctgtcatgtaaa
tcaaagccaaggttgtcaaagaacagccagtgaggccaggaaaga
aattggtcttgtggttttcatttttttcccccttgattgattata
ttttgtattgagatatgataagtgccttctatttcatttttgaat
aattcttcatttttataattttacatatcttggcttgctatataa
gattcaaaagagctttttaaatttttctaataatatcttacattt
gtacagcatgatgacctttacaaagtgctctcaatgcatttaccc
attcgttatataaatatgttacatcaggacaactttgagaaaatc
agtccttttttatgtttaaattatgtatctattgtaaccttcaga
gtttaggaggtcatctgctgtcatggatttttcaataatgaattt
agaatacacctgttagctacagttagttattaaatcttctgataa
tatatgtttacttagctatcagaagccaagtatgattctttattt
ttactttttcatttcaagaaatttagagtttccaaatttagagct
tctgcatacagtcttaaagccacagaggcttgtaaaaatataggt
tagcttgatgtctaaaaatatatttcatgtcttactgaaacattt
tgccagactttctccaaatgaaacctgaatcaatttttctaaatc
taggtttcatagagtcctctcctctgcaatgtgttattctttcta
taatgatcagtttactttcagtggattcagaattgtgtagcagga
taaccttgtatttttccatccgctaagtttagatggagtccaaac
gcagtacagcagaagagttaacatttacacagtgctttttaccac
tgtggaatgttttcacactcatttttccttacaacaattctgagg
agtaggtgttgttattatctccatttgatgggggtttaaatgatt
tgctcaaagtcatttaggggtaataaatacttggcttggaaattt
aacacagtccttttgtctccaaagcccttcttctttccaccacaa
attaatcactatgtttataaggtagtatcagaatttttttaggat
tcacaactaatcactatagcacatgaccttgggattacattttta
tggggcaggggtaagcaagtttttaaatcatttgtgtgctctggc
tcttttgatagaagaaagcaacacaaaagctccaaagggccccct
aaccctcttgtggctccagttatttggaaactatgatctgcatcc
ttaggaatctgggatttgccagttgctggcaatgtagagcaggca
tggaattttatatgctagtgagtcataatgatatgttagtgttaa
ttagttttttcttcctttgattttattggccataattgctactct
tcatacacagtatatcaaagagcttgataatttagttgtcaaaag
DONOR specific for g9 gRNA for the intron 1
RAG1 gene replacement strategy with long
right HA
INSERT
tgagcacacagttattacttggaaattgtgtacagactaagttga
agatgttaggagggaagattgtgggccaagtaacggggtgtatgt
gtgtgggtataggggggcagctgggatggaaatggggggctgctg
ctgctgctgcaccctggcctcctgaactaatgatatcactcacca
gaaactactgttcctgcactgtccaagccaccccaaactagtttg
tcaaaatgaatctgtgctgtgtggagggaggcacgcctgtagctc
tgatgtcagatggcaatgtgaattcctgacctcttctcttcctcc
cacaggccgccaccatggccgccagctttcctcctacactgggac
tgtctagcgcccctgacgagattcagcaccctcacatcaagttca
gcgagtggaagttcaagctgttcagagtgcggagcttcgagaaaa
cccctgaggaagcccagaaagagaagaaggacagcttcgagggca
agcccagcctggaacagtctcctgctgtgctggataaggccgacg
gccagaaacctgtgcctacacagcctctgctgaaggctcacccca
agttctccaagaagttccacgacaacgagaaggccagaggcaagg
ccatccaccaggccaatctgagacacctgtgccggatctgcggca
acagcttcagagccgacgagcacaatcggagataccctgtgcacg
gccctgtggatggaaagactctgggcctgctgcggaagaaagaaa
agagagccaccagctggcccgacctgatcgccaaggtgttcagaa
tcgacgtgaaggccgatgtggacagcattcaccccaccgagttct
gccacaactgctggtccatcatgcaccggaagttcagctctgccc
cttgcgaggtgtacttccccagaaacgtgaccatggaatggcacc
cacacacacccagctgcgacatctgcaacacagccagaagaggcc
tgaagcggaagtccctgcagcctaatctgcagctgagcaagaaac
tgaaaaccgtgctggaccaggccagacaggcccggcaaagaaaaa
gacgcgcccaggctagaatcagcagcaaggacgtgatgaagaaga
tcgccaactgcagcaagatccacctgagcaccaaactgctggccg
tggacttccctgagcacttcgtgaagtccatcagctgccagatct
gcgagcacatcctggccgatcctgtggaaacaaactgcaagcacg
tgttctgcagagtgtgcatcctgcggtgcctgaaagtgatgggca
gctactgccccagctgtagatacccttgcttccccaccgacctgg
aaagccctgtgaagtcctttctgagcgtgctgaacagcctgatgg
tcaagtgccccgccaaagaatgcaacgaggaagtgtccctggaaa
agtacaaccaccacatcagcagccacaaagagtccaaagaaatct
tcgtgcacatcaacaaaggcggcagaccccggcagcatctgctgt
ctcttacaagaagggcccagaagcaccggctgcgggaactgaagc
tgcaagtgaaggcctttgccgacaaagaggaaggcggcgacgtca
agagcgtgtgcatgaccctgtttctgctggccctgagagcccgga
atgagcatagacaggccgatgagctggaagccattatgcaaggca
aaggcagcggactgcagcctgctgtgtgtctggctatcagagtga
ataccttcctgagctgcagccagtaccacaagatgtaccggaccg
tgaaagccatcaccggcagacagatcttccagccactgcacgccc
tgagaaacgccgagaaagttctgctgcctggctaccaccacttcg
agtggcagcctccactgaagaacgtgtccagcagcaccgacgtgg
gcatcatcgatggactgtctggactgagcagcagcgtggacgatt
accccgtggacacaatcgccaagagattcagatacgacagcgccc
tggtgtctgccctgatggacatggaagaggacatcctggaaggca
tgcggagccaggacctggacgactatctgaacggccctttcaccg
tggtggtcaaagaaagctgtgacggcatgggcgacgtgtccgaga
aacacggatctggacctgtggtgccagagaaggccgtgcggttca
gcttcaccatcatgaagatcactatcgcccacagcagccagaacg
tgaaggtgttcgaggaagccaagcctaacagcgagctgtgctgca
agcctctgtgtctgatgctggccgacgagagcgatcacgagacac
tgaccgccattctgagccctctgatcgccgaacgggaagccatga
agtcctccgagctgatgctcgaactcggcggcatcctgagaacct
tcaagttcatcttccgcggcaccggctacgacgagaagctcgtta
gagaggtggaaggcctggaagcctctggcagcgtgtacatctgca
ccctgtgtgacgccaccagactggaagctagccagaacctggtgt
tccacagcatcaccagaagccacgccgaaaacctcgagagatacg
aagtgtggcggagcaacccctaccacgagagcgtggaagaactgc
gggatagagtgaagggcgtgtccgccaagcctttcatcgagacag
tgcctagcatcgacgccctgcactgcgatattggcaacgccgccg
aattctacaagatctttcagctggaaatcggcgaggtctacaaga
accccaacgcctctaaagaggaacggaagcgctggcaggccacac
tggataagcacctgagaaagaagatgaatctgaagcccatcatgc
ggatgaacggcaacttcgcccggaagctgatgaccaaagaaaccg
tggatgccgtgtgcgagctgatcccctctgaggaaagacacgagg
ccctgagggaactgatggacctgtacctgaagatgaagcccgtgt
ggcggtctagctgtcctgccaaagagtgccctgagtctctgtgcc
agtacagcttcaacagccagagattcgccgagctgctgagcacaa
agttcaagtacagatacgaggggaagatcacgaactacttccaca
agaccctggctcacgtgcccgagatcatcgagagagatggctcta
ttggcgcctgggcctctgagggcaatgagtctggcaacaagctgt
ttcggcggttcagaaagatgaacgccagacagagcaagtgctacg
agatggaagatgtgctgaagcaccactggctgtacaccagcaagt
acctgcagaaattcatgaacgcccacaacgccctcaagaccagcg
gctttaccatgaatcctcaggccagcctgggagatcctctgggca
ttgaggatagcctggaatcccaggacagcatggaattctgaaggc
atagaggactctctggaaagccaagattcaatggaattttaagta
gggcaaccacttatgagttggtttttgcaattgagtttccctctg
ggttgcattgagggcttctcctagcaccctttactgctgtgtatg
gggcttcaccatccaagaggtggtaggttggagtaagatgctaca
gatgctctcaagtcaggaatagaaactgatgagctgattgcttga
ggcttttagtgagttccgaaaagcaacaggaaaaatcagttatct
gaaagctcagtaactcagaacaggagtaactgcaggggaccagag
atgagcaaagatctgtgtgtgttggggagctgtcatgtaaatcaa
agccaaggttgtcaaagaacagccagtgaggccaggaaagaaatt
ggtcttgtggttttcatttttttcccccttgattgattatatttt
gtattgagatatgataagtgccttctatttcatttttgaataatt
cttcatttttataattttacatatcttggcttgctatataagatt
caaaagagctttttaaatttttctaataatatcttacatttgtac
agcatgatgacctttacaaagtgctctcaatgcatttacccattc
gttatataaatatgttacatcaggacaactttgagaaaatcagtc
cttttttatgtttaaattatgtatctattgtaaccttcagagttt
aggaggtcatctgctgtcatggatttttcaataatgaatttagaa
tacacctgttagctacagttagttattaaatcttctgataatata
tgtttacttagctatcagaagccaagtatgattctttatttttac
tttttcatttcaagaaatttagagtttccaaatttagagcttctg
catacagtcttaaagccacagaggcttgtaaaaatataggttagc
ttgatgtctaaaaatatatttcatgtcttactgaaacattttgcc
agactttctccaaatgaaacctgaatcaatttttctaaatctagg
tttcatagagtcctctcctctgcaatgtgttattctttctataat
gatcagtttactttcagtggattcagaattgtgtagcaggataac
cttgtatttttccatccgctaagtttagatggagtccaaacgcag
tacagcagaagagttaacatttacacagtgctttttaccactgtg
gaatgttttcacactcatttttccttacaacaattctgaggagta
ggtgttgttattatctccatttgatgggggtttaaatgatttgct
caaagtcatttaggggtaataaatacttggcttggaaatttaaca
cagtccttttgtctccaaagcccttcttctttccaccacaaatta
atcactatgtttataaggtagtatcagaatttttttaggattcac
aactaatcactatagcacatgaccttgggattacatttttatggg
gcaggggtaagcaagtttttaaatcatttgtgtgctctggctctt
ttgatagaagaaagcaacacaaaagctccaaagggccccctaacc
ctcttgtggctccagttatttggaaactatgatctgcatccttag
gaatctgggatttgccagttgctggcaatgtagagcaggcatgga
attttatatgctagtgagtcataatgatatgttagtgttaattag
ttttttcttcctttgattttattggccataattgctactcttcat
acacagtatatcaaagagcttgataatttagtt
Left HA
tgagcacacagttattacttggaaattgtgtacagactaagttga
agatgttaggagggaagattgtgggccaagtaacggggtgtatgt
gtgtgggtatagggtgggcagctgggatggaaatggggggctgct
gctgctgctgcaccctggcctcctgaactaatgatatcactcacc
agaaactactgttcctgcactgtccaagccaccccaaactagttt
gtcaaaatgaatctgtgctgtgtggagggaggcacgcctgtagct
ctgatgtcagatggcaatgt
Splice Acceptor
ctgacctcttctcttcctcccacagg
KOZAK
gccgccaccatg
coRAG1 CDS
atggccgccagctttcctcctacactgggactgtctagcgcccct
gacgagattcagcaccctcacatcaagttcagcgagtggaagttc
aagctgttcagagtgcggagcttcgagaaaacccctgaggaagcc
cagaaagagaagaaggacagcttcgagggcaagcccagcctggaa
cagtctcctgctgtgctggataaggccgacggccagaaacctgtg
cctacacagcctctgctgaaggctcaccccaagttctccaagaag
ttccacgacaacgagaaggccagaggcaaggccatccaccaggcc
aatctgagacacctgtgccggatctgcggcaacagcttcagagcc
gacgagcacaatcggagataccctgtgcacggccctgtggatgga
aagactctgggcctgctgcggaagaaagaaaagagagccaccagc
tggcccgacctgatcgccaaggtgttcagaatcgacgtgaaggcc
gatgtggacagcattcaccccaccgagttctgccacaactgctgg
tccatcatgcaccggaagttcagctctgccccttgcgaggtgtac
ttccccagaaacgtgaccatggaatggcacccacacacacccagc
tgcgacatctgcaacacagccagaagaggcctgaagcggaagtcc
ctgcagcctaatctgcagctgagcaagaaactgaaaaccgtgctg
gaccaggccagacaggcccggcaaagaaaaagacgcgcccaggct
agaatcagcagcaaggacgtgatgaagaagatcgccaactgcagc
aagatccacctgagcaccaaactgctggccgtggacttccctgag
cacttcgtgaagtccatcagctgccagatctgcgagcacatcctg
gccgatcctgtggaaacaaactgcaagcacgtgttctgcagagtg
tgcatcctgcggtgcctgaaagtgatgggcagctactgccccagc
tgtagatacccttgcttccccaccgacctggaaagccctgtgaag
tcctttctgagcgtgctgaacagcctgatggtcaagtgccccgcc
aaagaatgcaacgaggaagtgtccctggaaaagtacaaccaccac
atcagcagccacaaagagtccaaagaaatcttcgtgcacatcaac
aaaggcggcagaccccggcagcatctgctgtctcttacaagaagg
gcccagaagcaccggctgcgggaactgaagctgcaagtgaaggcc
tttgccgacaaagaggaaggcggcgacgtcaagagcgtgtgcatg
accctgtttctgctggccctgagagcccggaatgagcatagacag
gccgatgagctggaagccattatgcaaggcaaaggcagcggactg
cagcctgctgtgtgtctggctatcagagtgaataccttcctgagc
tgcagccagtaccacaagatgtaccggaccgtgaaagccatcacc
ggcagacagatcttccagccactgcacgccctgagaaacgccgag
aaagttctgctgcctggctaccaccacttcgagtggcagcctcca
ctgaagaacgtgtccagcagcaccgacgtgggcatcatcgatgga
ctgtctggactgagcagcagcgtggacgattaccccgtggacaca
atcgccaagagattcagatacgacagcgccctggtgtctgccctg
atggacatggaagaggacatcctggaaggcatgcggagccaggac
ctggacgactatctgaacggccctttcaccgtggtggtcaaagaa
agctgtgacggcatgggcgacgtgtccgagaaacacggatctgga
cctgtggtgccagagaaggccgtgcggttcagcttcaccatcatg
aagatcactatcgcccacagcagccagaacgtgaaggtgttcgag
gaagccaagcctaacagcgagctgtgctgcaagcctctgtgtctg
atgctggccgacgagagcgatcacgagacactgaccgccattctg
agccctctgatcgccgaacgggaagccatgaagtcctccgagctg
atgctcgaactcggcggcatcctgagaaccttcaagttcatcttc
cgcggcaccggctacgacgagaagctcgttagagaggtggaaggc
ctggaagcctctggcagcgtgtacatctgcaccctgtgtgacgcc
accagactggaagctagccagaacctggtgttccacagcatcacc
agaagccacgccgaaaacctcgagagatacgaagtgtggcggagc
aacccctaccacgagagcgtggaagaactgcgggatagagtgaag
ggcgtgtccgccaagcctttcatcgagacagtgcctagcatcgac
gccctgcactgcgatattggcaacgccgccgaattctacaagatc
tttcagctggaaatcggcgaggtctacaagaaccccaacgcctct
aaagaggaacggaagcgctggcaggccacactggataagcacctg
agaaagaagatgaatctgaagcccatcatgcggatgaacggcaac
ttcgcccggaagctgatgaccaaagaaaccgtggatgccgtgtgc
gagctgatcccctctgaggaaagacacgaggccctgagggaactg
atggacctgtacctgaagatgaagcccgtgtggcggtctagctgt
cctgccaaagagtgccctgagtctctgtgccagtacagcttcaac
agccagagattcgccgagctgctgagcacaaagttcaagtacaga
tacgaggggaagatcacgaactacttccacaagaccctggctcac
gtgcccgagatcatcgagagagatggctctattggcgcctgggcc
tctgagggcaatgagtctggcaacaagctgtttcggcggttcaga
aagatgaacgccagacagagcaagtgctacgagatggaagatgtg
ctgaagcaccactggctgtacaccagcaagtacctgcagaaattc
atgaacgcccacaacgccctcaagaccagcggctttaccatgaat
cctcaggccagcctgggagatcctctgggcattgaggatagcctg
gaatcccaggacagcatggaattctga
Right HA
aggcatagaggactctctggaaagccaagattcaatggaatttta
agtagggcaaccacttatgagttggtttttgcaattgagtttccc
tctgggttgcattgagggcttctcctagcaccctttactgctgtg
tatggggcttcaccatccaagaggtggtaggttggagtaagatgc
tacagatgctctcaagtcaggaatagaaactgatgagctgattgc
ttgaggcttttagtgagttccgaaaagcaacaggaaaaatcagtt
atctgaaagctcagtaactcagaacaggagtaactgcaggggacc
agagatgagcaaagatctgtgtgtgttggggagctgtcatgtaaa
tcaaagccaaggttgtcaaagaacagccagtgaggccaggaaaga
aattggtcttgtggttttcatttttttcccccttgattgattata
ttttgtattgagatatgataagtgccttctatttcatttttgaat
aattcttcatttttataattttacatatcttggcttgctatataa
gattcaaaagagctttttaaatttttctaataatatcttacattt
gtacagcatgatgacctttacaaagtgctctcaatgcatttaccc
attcgttatataaatatgttacatcaggacaactttgagaaaatc
agtccttttttatgtttaaattatgtatctattgtaaccttcaga
gtttaggaggtcatctgctgtcatggatttttcaataatgaattt
agaatacacctgttagctacagttagttattaaatcttctgataa
tatatgtttacttagctatcagaagccaagtatgattctttattt
ttactttttcatttcaagaaatttagagtttccaaatttagagct
tctgcatacagtcttaaagccacagaggcttgtaaaaatataggt
tagcttgatgtctaaaaatatatttcatgtcttactgaaacattt
tgccagactttctccaaatgaaacctgaatcaatttttctaaatc
taggtttcatagagtcctctcctctgcaatgtgttattctttcta
taatgatcagtttactttcagtggattcagaattgtgtagcagga
taaccttgtatttttccatccgctaagtttagatggagtccaaac
gcagtacagcagaagagttaacatttacacagtgctttttaccac
tgtggaatgttttcacactcatttttccttacaacaattctgagg
agtaggtgttgttattatctccatttgatgggggtttaaatgatt
tgctcaaagtcatttaggggtaataaatacttggcttggaaattt
aacacagtccttttgtctccaaagcccttcttctttccaccacaa
attaatcactatgtttataaggtagtatcagaatttttttaggat
tcacaactaatcactatagcacatgaccttgggattacattttta
tggggcaggggtaagcaagtttttaaatcatttgtgtgctctggc
tcttttgatagaagaaagcaacacaaaagctccaaagggccccct
aaccctcttgtggctccagttatttggaaactatgatctgcatcc
ttaggaatctgggatttgccagttgctggcaatgtagagcaggca
tggaattttatatgctagtgagtcataatgatatgttagtgttaa
ttagttttttcttcctttgattttattggccataattgctactct
tcatacacagtatatcaaagagcttgataatttagtt

### AAV6 Production and Titration

AAV6 production was performed by the vector core facility at the Telethon Institute of Genetics and Medicine (TIGEM), Pozzuoli (NA, Italy). Briefly, AAV vectors were produced by transient triple transfection of HEK293 cells by calcium phosphate. The following day, the medium was changed with serum-free DMEM and cells were harvested 72 hours after transfection. Cells were lysed by three rounds of freeze-thaw to release the viral particles and the lysate was incubated with DNAseI and RNAse I to eliminate nucleic acids. AAV vector was then purified by two sequential rounds of Cesium Cloride (CsCl2) gradient. For each viral preparation, physical titres (genome copies/mL) were determined by PCR quantification using TaqMan.

### Flow Cytometry Analysis

Flow cytometry analysis was performed to assess the recombination activity as GFP+ cells. Unstained and single-stained cells or compensation beads were used as negative and positive controls.

All samples were acquired through BD Canto (BD Bioscience) cytofluorimeter after Rainbow beads (Spherotech) calibration and raw data were collected through DIVA software (BD Biosciences). The data were subsequently analyzed with FlowJo software Version 9.3.2 (TreeStar) and the graphical output was automatically generated through Prism 6.0c (GraphPad software).

### Western Blot Assay

Cell protein lysate was prepared with RIPA buffer (ThermoFisher) following manufacturer instructions. The purified proteins were analyzed on Mini-PROTEAN TGX Gels (7.5%, Biorad), followed by Ponceau staining. For Western blot analysis, proteins were separated by SDS-PAGE under reducing conditions and then electrophoretically transferred onto polyvinylidine difluoride membranes (Bio Rad TransBlot Turbo). After protein transfer, the membranes were treated with the blocking buffer (TBS 1×, Tween20 1%, Non-fat milk 0.5%) followed by incubation with primary antibodies O/N at 4° (α-hRAG1 1:500-D36B3 Cell Signaling-, α-hp38 1:2000-9212 Cell Signaling-in blocking buffer). Following three washes with TBS Tween 1%, membranes were incubated 1 hour with HRP-conjugated goat anti-rabbit IgG (Cell Signalling). Bioluminescence was acquired by Bio Rad ChemiDoc.

### Example 2—RAG1 Gene Guide RNAs

### Results

### Generation of NALM6 and K562 Cas9 Cell Lines

To test our panel of Cas9 guide RNAs we generated two cell lines with inducible Cas9 expression. NALM6 and K562 cell lines were transduced with a lentiviral vector carrying the Cas9 cassette under the control of a TET-inducible promoter and a cassette that confers resistance to puromycin. After transduction with MOI 20 the two cell lines were kept in culture with puromycin 1.5 μg/ml for one week to select the transduced cells (FIG. 6A). After puromycin selection, a VCN 3.65 and a VCN 4.35 were verified by LTR specific ddPCR in NALM6 Cas9 and K562 Cas9 cell line respectively (FIG. 6B). Efficient Cas9 expression was also verified by RT-qPCR after two days of induction with scaling doses of doxycycline (FIG. 6C). The highest Cas9 expression was found at the dose of 1 μg/ml of doxycycline in both the cell lines.

### RAG1 Guide Selection

A panel of nine guides was first identified to target three non-repeated loci of RAG1 intron 1. In addition, three guides (gRNA 1,2,3) targeting the first 200 bp of RAG1 exon 2 were designed with the final aim to integrate the corrective RAG1 coding sequence in frame with the endogenous ATG. This strategy would exploit the endogenous splice acceptor thus preserving any putative endogenous splicing regulations (FIG. 7A).

Guides were electroporated as plasmid DNAs in K562 Cas9 and NALM6 Cas9 cell lines considering two different doses (100 ng/well and 200 ng/well.) Cas9 expression was induced the day before the electroporation and for the two following days by adding doxycycline (1 μg/ml) to the medium. Genomic DNA was extracted at day 7 and cutting frequency was evaluated measuring the percentage of NHEJ-mediated indel mutations by T7 nuclease assay (scheme shown in FIG. 7B).

The majority of the tested guides had good cutting frequency showing similar results in both cell lines. In particular, Guide 9 was the best performing guide targeting the intron with a cutting frequency up to 72.7% in K562 Cas9 and 78.5% in NALM6 Cas9. Similar cutting frequencies were also achieved by Guide 7, that showed a cutting frequency up to 67.5% in K562 Cas9 and 70.5% in NALM6 Cas9 cell lines. Guide 3 was the best performing guide targeting the exon with a cutting frequency up to 58.9% in K562 Cas9 (FIG. 7C) and 73.5% in NALM6 Cas9 (FIG. 7D). Of note, despite the higher expression of Cas9 expression in K562 Cas9 than in NALM6 Cas9 cell line, no difference in the overall cutting efficiency was observed. Cutting frequency was also tested in NALM6 WT using in vitro preassemble RNP of guide 9 and guide 3 at the dose of 25 or 50 pmol/well (FIG. 7E). Both guides retained a good activity, guide 3 reached up to 71.5% cutting frequency and guide 9 up to 78.5% at the higher dose of RNP.

### Off-Target Analysis

Preliminary in silico analysis demonstrated a promising off-target profile of guide 9 and showed that most likely off-targets fall in intronic regions thus suggesting a low risk of off-target related gene disruption events (FIG. 8A). A deeper characterization of the off-target profile of guide 7 and 9 was pursued by an unbiased off-target detection assay (GUIDE-seq, Tsai S Q, et al. Nat Biotechnol. 2015; 33(2):187-97). The analysis was performed using 50 pmol of High Fidelity Cas9 Nuclease V3 on K562 cells resulting in 45.3% and 64.6% cutting frequency by guide 7 and 9, respectively (FIG. 8B). We achieved low (8.4%) ODN integration for guide 7, but good frequency of integration for the guide 9 (38.2%) allowing the analysis of off-target in the samples (FIG. 8C). According to the analysis performed using the R Bioconductor package GUIDE-seq (Zhu L J, et al. BMC Genomics. 2017; 18(1)) using default parameters, no off-target site was identified for both guides. To deepen the investigation also to very weak potential off-targets, a second analysis with relaxed constraints was performed, and two off-target sites were found only for guide 7. These off-target sites fall into intronic or intergenic regions, with a number of mismatches >9 and at low frequency, indicating the low risk profile of guide 7. It is worth noting that no off-target sites were identified for Guide 9.

### Optimization of the Gene Editing Protocol on Human Cord Blood-CD34+ Cells

The editing procedure was then optimized in human CD34⁺ cells from cord blood (hCB-CD34). To this end, hCB-CD34 cells were thawed at day 0 and prestimulated for three days seeding 1×10⁶ cells/ml in StemSpan enriched with cytokines (hTPO 20 ng/ml, hlL6 20 ng/ml, hSCF 100 ng/ml, hFlt3-L 100 ng/ml, SR1 1 uM, UM171 50 nM).

At day 3, guides 3 and 9 were delivered by electroporation as in vitro preassembled RNPs and two doses were considered 25 and 50 pmol/well. To enhance cellular stability, chemical modification consisting in 2′-O-methyl 3′phosphorothioate were added at the last three terminal nucleotides at 5′ and 3′ ends of the guide RNAs.

Guide 9 retained an activity comparable to that verified in NALM6 and K562 cell lines, 73.9% cutting frequency was observed with 25 pmol/well and 80.1% with 50 pmol/well. Guide 3 displayed a lower activity in hCB-CD34 with a cutting frequency of 16.9% and 19.3% with 25 and 50 pmol/well respectively (FIG. 8F).

### Materials and Methods

### Cas9 Inducible Cell Lines

NALM6 Cas9 cell line was generated by transducing NALM6 cells with a lentiviral vector expressing Cas9 protein under the control of a TET-inducible promoter and with a vector that constitutively expresses the TET transactivator (Clackson T. Vol. 7, Gene Therapy. 2000. p. 120-5). When doxycycline is administered to the culture media, the TET transactivator can bind the promoter of the Cas9 and induce its expression in the cells. K562 Cas9 cell line was generated with the same vector. Doxycycline was administered 24 h before electroporation of the nuclease. Cell lines were maintained in RPMI 1640 medium supplemented with 10% FBS, glutamine and penicillin/streptomycin antibiotics (complete medium).

gRNA and RNP Assembly

Cas9 protein and custom RNA guides were purchased from Integrated DNA Technologies (IDT) and assembled following the manufacturer protocol. To enhance cellular stability, chemically modified guide RNAs were used. Briefly crRNA and trRNA were annealed heating them at 95° C. for 5 minutes and letting them slowly cool down at RT for 10 minutes. Cas9 protein was then incubated for 15 minutes at room temperature with the annealed guide RNA fragments, to assemble the ribonucleoprotein (RNP).

Guide sequences are shown in the table below:

| | |
|---|---|
| Guide 1 | TTTTCCGGATCGATGTGA |
| Guide 2 | GACATCTCTGCCGCATCTG |
| Guide 3 | GTGGGTGCTGAATTTCATC |
| Guide 4 | GATTGTGGGCCAAGTAACG |
| Guide 5 | GAAAGTCACTGTTGGTCGA |
| Guide 6 | CAATTTTGAGGTGTTCGTT |
| Guide 7 | GGGTTGAGTTCAACCTAAG |
| Guide 8 | TTAGCCTCATTGTACTAGC |
| Guide 9 | TCAGATGGCAATGTCGAGA |
| Guide 10 | GCAATTTTGAGGTGTTCGT |
| Guide 11 | ACCAGCCTCGGGATCTCAA |
| Guide 12 | TCAAATCAGTCGGGTTTCC |
| Guide RAG1KO | CCTTCTCAGCATTCCGA |
| Guide RAG1KO | AACATCTTCTGTCGCTGACT |

When used directly as RNA, the following guide sequences for guides 3, 7, 9 and RAG1 KO may be used:

| | |
|---|---|
| Guide 3 | TGTGGGTGCTGAATTTCATC |
| Guide 7 | GGGGTTGAGTTCAACCTAAG |
| Guide 9 | GTCAGATGGCAATGTCGAGA |
| Guide RAG1KO | GTACCTTCTCAGCATTCCGA |

### Mismatch Selective Endonuclease Assay

A T7 endonuclease (T7E1) assay was used to measure indels induced by NHEJ. Briefly, gDNA of gene edited cells was extracted and amplified by PCR with primers flanking the Cas9 RNP target site. The PCR product was denatured, slowly re-annealed and digested with T7 endonuclease (New England BioLabs) for 1 h, 37°. T7 nuclease only cut DNA at sites where there is a mismatch between the DNA strands, thus between re-annealed wild type and mutant alleles. Fragments were separated on LabChip GXII Touch High Resolution DNA Chip (PerkinElmer®) and analysed by the provided software. The ratio of the uncleaved parental fragment versus cleaved fragments was calculated and it gives a good estimation of NHEJ efficiency of the artificial nuclease. Calculation of % NHEJ: (sum cleaved fragment)/(sum cleaved fragments+parental fragment)×100. Primer used for NHEJ assay:

| | |
|---|---|
| Guides 1, 2, 3 FW | CCATAAACACTGTCAGAAGAGG |
| Guides 1, 2, 3 RV | GTGTTGCAGATGTCACAGG |
| Guides 4, 9, 11 FW | GAAGTGGTTCATGCAAGAGG |
| Guides 4, 9, 11 RV | GGATGAACATGGAGAAAGCAG |
| Guides 6, 7, 10 FW | GGGGAGAAATGTGTAGGGAAG |
| Guides 6, 7, 10 RV | CTCAAAAACAAAGAAATGGGCG |
| Guides 5, 8, 12 FW | ATAGGTGGATGGGATGATGG |
| Guides 5, 8, 12 RV | CCTCTTCTGACAGTGTTTATGG |
| Guides RAG1KO FW | GGAAAATGAATGCCAGGCAG |
| Guides RAG1KO RV | AGGTCATCATGCTGTACAAATG |
| Guides RAG1KO FW | TCCATGCTTCCCTACTGAC |
| Guides RAG1KO RV | CTCCCATTCCATCACAAGAC |

### Off-Target Analysis

In silico prediction of off-target profile was performed with COSMID (CRISPR Off-target Sites with Mismatches, Insertions, and Deletions) (Cradick T J, et al. Mol Ther—Nucleic Acids. 2014; 3(12):e214) to search genomes for potential CRISPR off-target sites. For GUIDE-Seq analysis K562 cells were electroporated with 50 pmol of High Fidelity Cas9 Nuclease V3 guide7 or guide 9 (as RNP) and dsODN to tag the breaks via an end-joining process consistent with NHEJ. dsODN integration sites in genomic DNA were precisely mapped at the nucleotide level using unbiased amplification and next-generation sequencing (Tsai S Q, et al. Nat Biotechnol. 2015; 33(2):187-97). Library construction and GUIDE-Seq sequencing were performed by Creative Biogen Biotechnology (NY, USA) using Unique Molecular Identifier (UMI) for tracking PCR duplicates. Quality checking and trimming were performed on the sequencing reads, using FastQC and Trim_galore, respectively. High quality reads were aligned against the human reference genome (GRCh38), using Bowtie2 (Langmead B, Salzberg S L. Nat Methods. 2012; 9(4):357-9) in the “very-sensitive-local” mode, in order to achieve optimal alignments. GUIDE-Seq data analysis was performed employing the R/Bioconductor package GUIDE-seq (Zhu L J, et al. BMC Genomics. 2017; 18(1)), and using UMI to deduplicate reads.

### Statistical Analysis

When normality assumptions were not met, non-parametric statistical tests were performed. Kruskal-Wallis test with multiple comparison post-test was performed when comparing more groups. When normality assumptions were met, two-way analysis of variance (ANOVA) was used. For repeated measures over time, two-way ANOVA with Bonferroni's multiple comparison post-test was utilized. Values are expressed as Mean±SD.

### Example 3—Evaluation of RAG1 Gene Guide RNAs Corrective and Editing Efficiencies

### Results

Evaluation of Corrective Efficiency of the Exon Strategies Exploiting g6 gRNA in NALM6.Rag1KO Cells

The g6 gRNA was selected for further evaluation, due to its efficient cutting and disruption of RAG1 function by non-homologous end joining (NHEJ). To assess the corrective efficiency of exon strategies exploiting g6 gRNA, we produced two AAV6 donors: a donor vector carrying short homology arms (HA) homologous to the flanking sequences of the g6 target site that is tested for the “exon 2 RAG1 gene targeting” strategy (hereafter called “targeting donor”); and a second donor vector carrying a short left HA (L-HA) homologous for the flanking sequence of the g6 target site and a long distal right HA (R-HA) homologous to the 3′UTR in order to favor HDR and gene replacement (hereafter called “replacement donor”). Both corrective donors were tested in combination with g6 gRNA in NALM6.Rag1 KO cells (FIG. 9A). Guide 6 gRNA was delivered into NALM6.Rag1 KO cells as an in vitro preassembled RNPs (50 pmol/well) followed by the transduction with the targeting or the replacement AAV6 donor.

The bulk NALM6 edited cells were subcloned to obtain single clones that were analysed by digital drop PCR (ddPCR) to identify mono- or bi-allelic edited alleles (FIG. 9A). We screened 640 clones by ddPCR and we identified 9 mono-allelic and 1 bi-allelic clones (clone 11) edited by g6 and the targeting donor and 7 mono-allelic clones edited by g6 and the replacement donor (FIG. 9B).

Next, we tested the recombination activity of mono-allelic and bi-allelic clones edited with the two different strategies. Single clones were transduced with a LV carrying an inverted GFP cassette which is recombined in the presence of a functional RAG1 protein (FIG. 9C-D). All NALM6.Rag1 KO edited clones showed high levels of LV-transduction efficiency (FIG. 9C) and, importantly, improved or restored levels of recombination activity reaching the frequency of NALM6 wild-type (NALM6-WT) cells after serum starvation. In particular, GFP+ cells were higher in the group of clones edited with the replacement donor strategy than in clones edited by the targeting strategy (FIG. 9E).

RAG1 expression induced by the donor cassette was assessed by RT-qPCR in parallel and serum starvation was exploited to synchronize edited cells in G1 cell cycle phase when the recombination activity is high. We observed a statistically significant increase of RAG1 CDS expression in starved edited clones as compared to not starved edited clones (FIG. 9F-G). The highest level of RAG1 CDS expression was observed in the bi-allelic edited clone 11 with a fold induction of 8 upon starvation (FIG. 9F-G), which is similar to the fold induction of the endogenous RAG1 expression observed in NALM6-WT cells (FIG. 9H).

Evaluation of editing efficiency of the exon strategies exploiting g6 gRNA in human HSPC

Overall, these data indicate that both editing strategies are able to obtain good level of RAG1 expression and recombination activity prompting us to evaluate the impact of these strategies on human hematopoietic stem and progenitor cells (HSPC). Mobilized peripheral blood (mPB) CD34+ cells from two independent healthy donors (HDs) were electroporated with g6 and Cas9 as RNP (50 pmol) in the presence of the combination of editing enhancers (GSE56 and Ad5-E4orf6/7) and then transduced with the targeting or the replacement donor (FIG. 10A).

Gene editing efficiency was assessed by molecular analysis, evaluation of stemness markers by flow cytometry analysis, the ability to form colonies by CFU assay and the T cell differentiation potential by exploiting the artificial thymic organoid (ATO) system. We observed a higher proportion of edited alleles in the targeting donor cassette setting as compared to the replacement strategy in both HD samples (mean percentage of edited alleles: 7.6% for targeting, 4.4% for replacement) (FIG. 10B).

We observed similar impact of the two donor cassettes on the viability of edited cells in terms of cellular growth with a tendency to a lower growth rate of replacement-edited HSPC than targeting-edited cells (FIG. 10C). In parallel, analysis of GE impact of both strategies on HSPC distribution showed no gross alterations of cell composition with preservation of the most primitive CD34+CD133+CD90+ cells (FIG. 10D).

We exploited the ATO platform to evaluate the differentiation capacity of edited and unedited CD34+ cells. Similar frequencies of T cell precursors and CD3⁺ cells expressing TCRα/β were obtained in ATOs seeded with unedited and edited cells using the targeting or the replacement setting (FIG. 10E-F). Molecular analysis of edited alleles showed similar frequencies in the bulk population and in sorted double negative and double positive cellular populations differentiated in ATOs.

Screening of New Panel of gRNAs and Corrective Donor Constructs for RAG1 Exonic Strategies

Results obtained with g6 gRNA prompted us to investigate a panel of 8 gRNAs mapping at the 5′ region of the gene and targeting the same region of g6 gRNA. As for previous gRNA panel tested, the additional 8 gRNA target the last internal nonstandard Methionines (M) at 5′ of RAG1 to achieve RAG1 inactivation by NHEJ and favour selective advantage of cells edited by HDR over uncorrected cells (FIG. 11A).

These 8 gRNAs and g14 (g14×KO), this latter already designed to inactivate the catalytic core of RAG1 gene in the exon 2, were electroporated in NALM6-WT cells with the final aim to assess their cutting efficiency and the impact of RAG1 disruption in terms of recombination activity by means of LV GFP inverted cassette (FIG. 11B). All gRNAs showed high levels of efficiency with no differences between the use of single gRNAs (sgRNA Synthego) and two-part gRNAs (gRNA IDT) (FIG. 11C). Importantly, the analysis of recombination activity in edited NALM6-WT cells and in the NALM6 line in which RAG1 gene was inactive (NALM6-Rag1KO) showed levels of reduced recombination activity for most of the gRNAs tested (FIG. 11D).

These data prompted us to further test these sgRNA in CD34⁺ cells in terms of cutting efficiency and RAG1 disruption in ATO platform (FIG. 12A). Hematopoietic stem and progenitor cells derived from mPB of two HDs were thawed at day 0 and prestimulated for three days in StemSpan enriched with early active cytokines and compounds for stemness preservation. At day 3, each sgRNA was delivered as an in vitro preassembled RNP (25 or 50 μmol) by electroporation. Four and seven days after the editing, cells were collected, and DNA was extracted to measure the cutting efficiency of each gRNA by performing the NHEJ assay (T7 mismatch selective endonuclease assay). In parallel, we tested as controls g5 and g6 selected from the first gRNA panel and g9 which targets the RAG1 intron1 site (FIG. 12A). Analysis of cutting efficiency showed that g11 and g13 sgRNAs achieved the highest levels of NHEJ as compared to other sgRNAs in HD CD34+ cells (FIG. 12B).

To verify the capability of new sgRNAs in inactivating RAG1 gene, we tested the effect of these sgRNAs on T cell differentiation in the ATO system that showed a dramatic reduction of CD3⁺ TCRab⁺ cells frequency especially in cells edited by g8, g10, g11, g12, and g13 (FIG. 12C-D). Kinetics of T cell differentiation confirmed these data showing very low fraction of CD3⁺ TCRab⁺ cells over time in ATOs obtained with cells treated by g13 and g11 (9.81% and 2.34% for g11 and g13 respectively, 6 weeks post seeding) (FIG. 12E), confirming their cutting efficiencies in ATO cells (FIG. 12F).

Overall, these findings indicate g6, g13 and g11 as promising sgRNAs able to achieve good levels of cutting efficiency thus leading to impaired recombination activity and T cell differentiation.

Evaluation of corrective and editing efficiencies of the exon strategies exploiting g11 and g13 gRNAs in NALM6.Rag1KO cells and human mPB-CD34+ cells

These data prompted us to design and produce novel corrective donor templates. To this aim, we designed and generated the following additional donor cassettes specific for each sgRNA and optimized in HA lengths (FIG. 13):
for g6 sgRNA:
1) a second replacement donor cassette carrying the codon optimized RAG1 flanked by a L-HA of 243 bp and a right R-HA long 900 bp was designed to verify if a shorter R-HA for the replacement cassette could improve HDR efficiency and decrease the impact on HSPC biology. Thus, this donor construct will be compared to the targeting (2) and the replacement donor cassettes (3) previously generated and tested on HSPC;
for g13 sgRNA:
1) the targeting donor cassette carrying the codon optimized RAG1 flanked by a L-HA of 522 bp and a R-HA of 500 bp; 2) the replacement donor cassette carrying the codon optimized RAG1 flanked by a L-HA of 522 bp and a right R-HA long 1189 bp;
for g11 sgRNA:
1) the targeting donor cassette carrying the codon optimized RAG1 flanked by a L-HA of 536 bp and a right R-HA homology arm of 500 bp; 2) the replacement donor cassette carrying the codon optimized RAG1 flanked by a L-HA of 536 bp and a R-HA long 1189 bp.

Remarkably the replacement donor cassette designed for g13 can be exploited also for g7 and g10.

Next, we applied the GE platform including g11, g13 and g6 with the corresponding targeting and replacement corrective donors on NALM6-Rag1 KO cells to assess the efficiency of GE and the ability to induce recombination activity (FIG. 14A). Molecular analysis assessed by ddPCR performed on bulk edited and unedited NALM6-Rag1 KO cells demonstrated a frequency of 9.5% and 6.4% in the presence of g11 with the targeting donor and replacement donor respectively, while similar frequencies (8.9% and 9%) were observed for g13 using both corrective donors (FIG. 14B). HDR efficiencies obtained by using g11 and g13 were higher than those observed in cells edited by g6 (FIG. 14B).

In parallel, we tested the recombination activity induced by the new sgRNA/corrective donor sets exploiting the LV carrying an inverted GFP cassette which is recombined in the presence of a functional RAG1 protein. The analysis was performed on bulk NALM6-Rag1KO cells edited with the two strategies (g11 versus g13, and the corresponding corrective donors (Targeting versus Replacement)). To synchronize cell cycle phase in G1 phase of cell cycle when recombination activity is high, edited cells were kept in culture in the absence of serum (serum starvation) or in presence of the inhibitor of cyclin-dependent kinase 4 and 6 (CDK4/6i), a cell cycle inhibitor known to arrest the cell cycle during transition from cell growth (G1) to DNA synthesis (S) phase (FIG. 14C). Similar frequencies of GFP positive cells were detected at day 4 and 7 irrespectively to the GE platforms. Importantly, the levels of recombination activity achieved by the two sgRNA (g11 and g13) and the two exon strategies (targeting and replacement) were in line with the levels of HDR obtained in bulk edited NALM6-Rag1 KO cells (FIG. 14C).

These data provided evidence of RAG1 correction mediated by exon strategies exploiting g11 or g13 sgRNAs and prompted us to isolate single gene edited NALM6 clones to confirm these observations.

Next, we tested the GE platform including g6, g11 and g13 and the corresponding AAV6 targeting and replacement donors in the presence of gene editing enhancers (GSE56 and Ad5-E4orf6/7) in mPB CD34⁺ cells obtained from two independent HDs (FIG. 15A). The proportion of edited alleles, analyzed by ddPCR on bulk untreated and edited CD34⁺ cells 4 days after the editing, was 2-fold and 3-fold higher in g11- or g13-edited cells, respectively, as compared to g6-edited cells (mean of HDR of targeting and replacement GE: 24.5% for g11, 34% for g13 and 10% for g6) (FIG. 15B). Moreover, we confirmed higher NHEJ levels in HSPC edited by g11 and g13 than g6 (FIG. 15C), likely suggesting that the improved HDR is due to the increase of their cutting efficiencies. The optimized HA length of donor vectors specific for g11 and g13 could also contribute to the increased HDR efficiency.

Analysis of HSPC composition of mPB-CD34⁺ cells undergoing GE four days after did not show gross changes as respect to unedited cells (FIG. 15D). Evaluation of CFU before and after GE showed a reduced number of colonies in case of g6 particularly in the presence of replacement strategy (FIG. 15E). Moreover, the use of a replacement donor with a shorter R-HA for g6 GE improved the impact on the clonogenic potential (FIG. 15E) but did not increase HDR efficiency as compared to previously tested donors specific for g6 target site (FIG. 15B).

Overall, the levels of cutting efficiency and HDR in association with recombination activity achieved with g11 and g13 indicate promising results.

Evaluation of Corrective Efficiency of the Exon Gene Editing Strategy Exploiting g11 and g13 sgRNAs in NALM6.Rag1KO Cells

We selected g11 and g13 sgRNAs as the best performing sgRNAs in terms of cutting efficiency, disruption of RAG1 function by non-homologous end joining (NHEJ), and HDR efficiency in NALM6.Rag1 KO cells (FIG. 12D and FIG. 14B) and MPB-HPSCs (FIG. 15B-C).

To assess the corrective efficiency of exon strategies exploiting new sgRNAs, g11 or g13 sgRNAs were delivered into NALM6.Rag1 KO cells as in vitro preassembled RNPs followed by the transduction with the targeting or the replacement AAV6 donors at a dose of 10⁴. The bulk NALM6 edited cells were subcloned to obtain single clones that were analysed by digital dropplet PCR (ddPCR) to identify mono- or bi-allelic edited alleles (FIG. 16A). We screened 370 clones by ddPCR and selected 5 mono-allelic clones and 1 bi-allelic clones (clone 69) edited by g11 and the targeting donor, while we selected 6 mono-allelic clones for the other experimental groups. Next, we tested the recombination activity of mono-allelic and bi-allelic clones edited with the two different strategies. Single clones were transduced with a LV carrying an inverted GFP cassette which is recombined in presence of a functional RAG1 protein. All NALM6.Rag1 KO edited clones showed high levels of LV-transduction efficiency and improved or restored RAG1-mediated recombination activity reaching the frequency of NALM6 wild-type (NALM6-WT) cells (FIG. 16B). In parallel, RAG1 expression induced by the donor cassette was assessed by RT-qPCR in parallel and serum starvation was exploited to synchronize edited cells in G1 cell cycle phase when the recombination activity is high. We observed the increase of RAG1 CDS expression in starved edited clones as compared to not starved edited clones (FIG. 16C). Interestingly, the highest level of RAG1 CDS expression was observed in the bi-allelic edited (clone 69 edited by g11 and the targeting donor) with a fold induction similar to that of the endogenous RAG1 expression observed in NALM6-WT cells (FIG. 9H).

Data on NALM6.Rag1 KO cells indicate that both editing strategies are able to obtain good level of RAG1 expression and recombination activity.

Evaluation of Editing and Correction Efficiency of g11- and g13-Mediated Gene Editing in Human HSPCs Derived from Healthy Donor and RAG1-Patient

Mobilized peripheral blood (MPB) CD34+ cells from two independent healthy donors (HDs) and a hypomorphic RAG1 patient were electroporated with g11 or g13 and Cas9 as RNP (50 pmol) in presence of the combination of editing enhancers (GSE56 and Ad5-E4orf6/7) and then transduced with the targeting or the replacement donor (dose 10⁴) (FIG. 17A). Gene editing efficiency was assessed by molecular analysis of HDR, evaluation of stemness markers by flow cytometry analysis, T cell differentiation potential by exploiting the artificial thymic organoid (ATO) system and engraftment and T and B cell differentiation correction potential by xenotransplant assay in NSG mice (FIG. 17A).

Both exon strategies resulted in high levels of homology directed repair (HDR) efficiency in HD and Patient-derived HSPCs in vitro, with a tendency to a higher proportion of edited alleles in g13-edited cells (35.5% HD, 32% RAG1-patient; average between cells edited with targeting and replacement donor) than g11-edited cells (24.5% HD, 23% RAG1-patient; average between cells edited with targeting and replacement donor) (FIG. 17B). This finding is in line with the higher cutting efficiency of g13 than g11 (FIGS. 12B and 15C). In parallel, analysis of gene editing impact of both strategies on HSPC distribution showed no gross alterations of cell composition with preservation of the most primitive CD34+CD133+CD90+ cell subset (FIG. 17C).

We exploited the ATO platform to evaluate the differentiation capacity of edited and unedited CD34+ cells. Of note, the RAG1-patient is an adult patient presenting combined immunodeficiency with granuloma and autoimmunity (CID-G/AI) due to missense RAG1 mutations (C1228T; G1520A) allowing residual development of B and T cells. As expected, untreated patient-derived HSPCs did not differentiate into T cells in ATO platform due to the missense RAG1 mutations (FIG. 17D). Importantly, both corrective donors were able to rescue RAG1 function and overcome the T cell block (FIG. 17D-E). A high proportion of TCRα/β3+CD3+ cells were generated in ATOs seeded with HD-HSPC edited with g13 and the targeting or the replacement donor (FIG. 17E), confirming the efficacy of the exonic gene editing strategies in correcting human RAG1 defects. To further investigate the robustness of T cell development rescue, we analyzed the TCRβ repertoire of bulk or sorted TCRα/β+CD3+ cells ATO cells by TCRB immunoSEQ assay (Adaptive Biotechnologies). We assessed the Simpson Complexity index, which measures the sample clonality, ranging from 0, for a properly diverse population to 1, for a monoclonal population. We obtained comparable values among samples closed to 0, indicating that ATO-T cells differentiated from edited HD and RAG1-HSPC showed a diverse TRB repertoire (FIG. 17F). Preliminary analysis of the top 10 productive rearrangements showed the absence of dominant clones in all samples (FIG. 17G).

To evaluate in vivo gene correction in terms of lymphoid differentiation, which is limited in hypomorphic RAG1 patients, we transplanted untreated and edited RAG1-patient HSPCs in sub-lethally irradiated NSG mice. Kinetics of human cell engraftment was monitored over time by flow cytometric analysis till the termination of the experiment. We confirmed the engraftment of human untreated and edited HSPCs in NSG mice with no great differences between treated and untreated cells confirming that engraftment capability was not affected by the editing protocol (FIG. 18A). Of note, molecular analysis performed by ddPCR assay revealed high HDR efficiency (ranging from stable 18.7% to 64.1%) stable over time in vivo (FIG. 18B). Similar targeting frequencies were observed in HD and Patient's samples (HDR average and median values calculated intra-sample for all time points: 42.2% and 43.7% HD g13-targeting, 46.2% and 44% HD g13-replacement, 40.7% and 44.2% RAG1-patient g13-targeting, 44.2% and 53.5% RAG1-patient g13-replacement).

With regard to peripheral blood composition, NSG mice transplanted with treated HD cells showed no major skewing in the subpopulation composition and a comparable frequency of B, T and myeloid cells was observed in mice receiving treated or untreated cells, confirming that multilineage differentiation was not impaired (FIG. 18C). Mice transplanted with untreated patient cells showed low B cell frequency when compared to HD-treated mice, in line with the immune phenotype of patients carrying hypomorphic mutations (Delmonte O M, et al. Blood. 2020; 135(9):610-9). Importantly, both targeting and replacement strategies rescued peripheral B cell frequencies in mice treated with edited-patient HSPCs, reaching values of HD-treated mice (FIG. 18C) and showing kinetics of cell repopulation similar to that observed HD-treated mice. These findings demonstrate the efficacy of the exonic gene editing strategies in correcting RAG1 function and overcoming the B-cell differentiation block. The improved B cell output in mice treated with edited patient-HSPCs was associated with the redistribution of myeloid cells which remained high only when untreated patient-HSPCs were injected (FIG. 18C). T cell differentiation and output was not affected by the editing procedure and the two correction platforms (FIG. 18C).

To evaluate B and T cell lymphopoiesis, we collected central lymphoid organs 18 weeks after the transplant. Analysis of the immune cell composition in bone marrow confirmed the multilineage differentiation of untreated and edited HD and patient cells (FIG. 18D). RAG1 gene editing allowed to strongly improve B cell compartment in terms of frequencies (FIG. 18D) and B cell lymphopoiesis (FIG. 18E). Indeed, we observed a reduction of progenitor B cell (PRO-B and PRE-BI cells) subsets associated with the relative expansion of the last steps of B cell development in mice treated with edited patient-HSPCs as compared to untreated cells (FIG. 18E). Targeting efficiency evaluated in bone marrow cells and in the thymus showed engraftment of edited HD and patient cells (FIGS. 18F and 18H). There was evidence of improved thymopoiesis derived from the increased proportion of TCRα/β+CD3+ cells in mice treated with edited patient-HSPCs as compared to mice treated with mutated HSPCs (FIG. 18G).

Overall, these results strongly support the therapeutic potential of gene editing strategy in correcting RAG1 deficiency.

Off-Target Analysis of g11 and g13 sgRNAs

Preliminary in silico analysis demonstrated promising off-target profiles of g11 and g13 sgRNAs and showed that the majority of off-targets fall in non-exonic genomic regions thus suggesting a low risk of off-target related gene disruption events. A deeper characterization of off-target profiles of g11 and g13 sgRNAs was pursued by an unbiased off-target detection assay (GUIDE-seq, Tsai S Q, et al. Nat Biotechnol. 2015; 33(2):187-97) (FIG. 19A). The analysis was performed using 50 pmol of High Fidelity Cas9 Nuclease V3 on K562 cells resulting in high cutting frequency (FIG. 19B). Consistently, we achieved high ODN integration for g11 and g13 (49.9% and 76.0%, respectively), allowing the analysis of off-targets in the samples (FIG. 19C). According to the analysis performed using the R Bioconductor package GUIDE-seq (Zhu L J, et al. BMC Genomics. 2017; 18(1)) using default parameters, only few off-target sites were identified for both guides, especially for g13 (FIG. 19D). These off-target sites fall into intronic or intergenic regions, with a high number of mismatches, indicating the low risk profiles of g11 and g13 sgRNAs.

### Materials and Methods

### NHEJ Efficiency

Indels induced by NHEJ were measured by a mismatch selective endonuclease assay using the T7 endonuclease (T7E1). Briefly, gDNA of gene edited cells was extracted and amplified by PCR with primers flanking the Cas9 RNP target site. The PCR product was denatured, slowly re-annealed and digested with T7 endonuclease (New England BioLabs) for 1 h, 37° C. T7 nuclease only cut DNA at sites where there is a mismatch between the DNA strands, thus between re-annealed wild type and mutant alleles. Fragments were separated on 4200 Tape Station System (Agilent) and analyzed by the provided software. The ratio of the uncleaved parental fragment versus cleaved fragments was calculated as percentage of NHEJ: (sum cleaved fragment)/(sum cleaved fragments+ parental fragment)×100.

Primers used for NHEJ assay are shown below according to the gRNA specificity.

Primers specific for “g6 M2 ex2”, “g11 exon2 M2/3” and “g13 exon2 M2/3” gRNAs (Exonic strategy):

FW: AGCCAACCTTCGACATCTCT
RV: CAAAGTGCTCTGGGAAGTCC

Digital droplet PCR

For HDR digital droplet PCR (ddPCR) analysis, 5-50 ng of gDNA were analyzed using the QX200 Droplet Digital PCR System (Bio-Rad) according to the manufacturer's instructions. HDR ddPCR primers were designed on the junction between the vector sequence and the targeted locus. Human TELO were used for normalization. We optimized a EvaGreen-based ddPCR protocol to detect dsDNA (QX200 EvaGreen Digital PCR Supermix). The percentage of cells harboring biallelic integration was calculated with the following formula: (concentration (copies/μl) of target+ droplets/concentration of TELO+ droplets)×100.

Primers and Probes used for ddPCR assay are the following:

| | |
|---|---|
| g6 FW | TCAGAATGGAAATTTAAGCTGTTC |
| g11-g13 FW | CACCCACCTTGGGACTCAGTTCT |
| g6-g11-g13 RV | TCCGCTTCAGGCCTCTTCT |

Optimized PCR program for assessing HDR induced by “g6 M2 ex2” (40 cycles):
95° C.×5 min
40×95° C.×30 sec
55° C.×1 min
72° C.×2 min
4° C.×4 min
90° C.×5 min
4° C. hold

Optimized PCR program for assessing HDR induced by “g11 M2 ex2/3” and “g13 M2 ex2/3” (40 cycles):
95° C.×5 min
40×95° C.×30 sec
62° C.×1 min
72° C.×2 min
4° C.×4 min
90° C.×5 min
4° C. hold

### Donor Constructs

DONOR specific for “g6 M2 ex2 RAG1” gRNA for the exon 2 RAG1 gene targeting
strategy
INSERT
tgagatcctttgaaaagacacctgaagaagctcaaaaggaaaagaaggattcctttgaggggaaaccctctctggagcaatct
ccagcagtcctggacaaggctgatggtcagaagccagtcccaactcagccattgttaaaagcccaccctaagttttcaaagaaa
tttcacgacaacgagaaagcaagaggcaaagcgatccatcaagccaaccttcgacatctctgccgcatctgtgggaattctttta
gagctgatgagcacaacaggagatatccagtccatggtcctgtggatggtaaaaccctaggccttttacgaaagaaggaaaag
agagctacttcctggccggacctcattgccaaggttttccggatcgatgtgaaggcagatgttgactcgatccaccccactgagttc
tgccataactgctggagcatcatgcacaggaagtttagcagtgccccatgtgaggtttacttcccgaggaatgtcactatggaatg
gcaccctcacacacccagctgcgacatctgcaacacagccagaagaggcctgaagcggaagtccctgcagcctaatctgca
gctgagcaagaaactgaaaaccgtgctggaccaggccagacaggcccggcaaagaaagagaagggcccaagccagaat
cagcagcaaggacgtgatgaagaagatcgccaactgcagcaagatccacctgagcaccaaactgctggccgtggacttccct
gagcacttcgtgaagtccatcagctgccagatctgcgagcacatcctggccgatcctgtggaaacaaactgcaagcacgtgttct
gcagagtgtgcatcctgcggtgcctgaaagtgatgggcagctactgcccctcctgcagatacccttgcttccccaccgatctggaa
agccctgtgaagtccttcctgagcgtgctgaacagcctgatggtcaagtgccccgccaaagaatgcaacgaggaagtgtccctg
gaaaagtacaaccaccacatcagcagccacaaagagtccaaagaaatcttcgtgcacatcaacaaaggcggcagaccccg
gcagcatctgctgtctcttacaagacgggcccagaagcaccggctgagagaactgaagctgcaagtgaaggcctttgccgaca
aagaggaaggcggcgacgtcaagagcgtgtgcatgaccctgtttctgctggccctgagagcccggaatgagcatagacaggc
cgatgagctggaagccatcatgcaaggcaaaggcagcggactgcagcctgctgtgtgtctggctatcagagtgaacaccttcct
gtcctgcagccagtaccacaagatgtaccggaccgtgaaggccattaccggcagacagatcttccagcctctgcacgccctga
gaaacgccgagaaagttctgctgcctggctaccaccacttcgagtggcagcctccactgaagaacgtgtccagcagcaccgac
gtgggcatcatcgatggactgagcggactgtctagcagcgtggacgactaccccgtggacacaatcgccaagcggttcagata
cgacagcgccctggtgtctgccctgatggacatggaagaggacatcctggaaggcatgcggagccaggacctggacgattac
ctgaacggccctttcaccgtggtggtcaaagaaagctgtgacggcatgggcgacgtgtccgagaaacacggatctggacctgt
ggtgccagagaaggccgtgcggttcagcttcaccatcatgaagatcactatcgcccacagcagccagaacgtgaaagtgttcg
aggaagccaagcctaacagcgagctgtgctgcaagcctctgtgtctgatgctggccgacgagagcgatcacgagacactgac
cgccattctgagccctctgatcgccgaacgggaagccatgaagtcctccgagctgatgctcgaactcggcggcatcctgagaac
cttcaagttcatcttccgcggcaccggctacgacgagaagctcgttagagaggtggaaggcctggaagcctctggcagcgtgta
catctgcaccctgtgtgacgccaccagactggaagctagccagaacctggtgttccacagcatcaccagaagccacgccgaa
aacctggaaagatacgaagtgtggcggagcaacccctaccacgagagcgtggaagaactgcgggatagagtgaagggcgt
gtccgccaagcctttcatcgagacagtgcctagcatcgacgccctgcactgcgatattggcaacgccgccgaattctacaagatc
tttcagctggaaatcggcgaggtgtacaagaaccccaacgcctctaaagaggaacggaagcgctggcaggccacactggat
aagcacctgagaaagaagatgaatctgaagcccatcatgaggatgaacggcaacttcgcccggaagctgatgaccaaagaa
accgtggatgccgtgtgcgagctgatcccctctgaggaaagacacgaggccctgcgggaactgatggacctgtacctgaagat
gaagcccgtgtggcggtctagctgtcctgccaaagagtgccctgagtctctgtgccagtacagcttcaacagccagagattcgcc
gagctgctgtccaccaagttcaagtacagatacgagggcaagatcaccaactacttccacaagaccctggctcacgtgcccga
gatcatcgagagagatggctctattggcgcctgggcctctgagggcaatgagtctggcaacaagctgttccggcggttccgcaa
gatgaacgccagacagagcaagtgctacgagatggaagatgtgctgaagcaccactggctgtacaccagcaagtacctgca
gaaattcatgaacgcccacaacgccctcaagaccagcggctttaccatgaatcctcaggccagcctgggcgaccctctgggaa
ttgaggatagcctggaatcccaggacagcatggaattctgataagagtggcacccccacacaccatcctgtgacatctgcaaca
ctgcccgtcggggactcaagaggaagagtcttcagccaaacttgcagctcagcaaaaaactcaaaactgtgcttgaccaagca
agacaagcccgtcagcgcaagagaagagctcaggcaaggatcagcagcaaggatgtcatgaagaagatcgccaactgca
gtaagatacatcttagtaccaagctccttgcagtggacttcccagagc
Left HA
tgagatcctttgaaaagacacctgaagaagctcaaaaggaaaagaaggattcctttgaggggaaaccctctctggagcaatct
ccagcagtcctggacaaggctgatggtcagaagccagtcccaactcagccattgttaaaagcccaccctaagttttcaaagaaa
tttcacgacaacgagaaagcaagaggcaaagcgatccatcaagccaaccttcgacatctctgccgcatctgtgggaattctttta
gagctgatgagcacaacaggagatatccagtccatggtcctgtggatggtaaaaccctaggccttttacgaaagaaggaaaag
agagctacttcctggccggacctcattgccaaggttttccggatcgatgtgaaggcagatgttgactcgatccaccccactgagttc
tgccataactgctggagcatcatgcacaggaagtttagcagtgccccatgtgaggtttacttcccgaggaatgtcactatg
coRAG1 CDS
gaatggcaccctcacacacccagctgcgacatctgcaacacagccagaagaggcctgaagcggaagtccctgcagcctaat
ctgcagctgagcaagaaactgaaaaccgtgctggaccaggccagacaggcccggcaaagaaagagaagggcccaagcc
agaatcagcagcaaggacgtgatgaagaagatcgccaactgcagcaagatccacctgagcaccaaactgctggccgtgga
cttccctgagcacttcgtgaagtccatcagctgccagatctgcgagcacatcctggccgatcctgtggaaacaaactgcaagcac
gtgttctgcagagtgtgcatcctgcggtgcctgaaagtgatgggcagctactgcccctcctgcagatacccttgcttccccaccgat
ctggaaagccctgtgaagtccttcctgagcgtgctgaacagcctgatggtcaagtgccccgccaaagaatgcaacgaggaagt
gtccctggaaaagtacaaccaccacatcagcagccacaaagagtccaaagaaatcttcgtgcacatcaacaaaggcggcag
accccggcagcatctgctgtctcttacaagacgggcccagaagcaccggctgagagaactgaagctgcaagtgaaggcctttg
ccgacaaagaggaaggcggcgacgtcaagagcgtgtgcatgaccctgtttctgctggccctgagagcccggaatgagcatag
acaggccgatgagctggaagccatcatgcaaggcaaaggcagcggactgcagcctgctgtgtgtctggctatcagagtgaac
accttcctgtcctgcagccagtaccacaagatgtaccggaccgtgaaggccattaccggcagacagatcttccagcctctgcac
gccctgagaaacgccgagaaagttctgctgcctggctaccaccacttcgagtggcagcctccactgaagaacgtgtccagcag
caccgacgtgggcatcatcgatggactgagcggactgtctagcagcgtggacgactaccccgtggacacaatcgccaagcgg
ttcagatacgacagcgccctggtgtctgccctgatggacatggaagaggacatcctggaaggcatgcggagccaggacctgg
acgattacctgaacggccctttcaccgtggtggtcaaagaaagctgtgacggcatgggcgacgtgtccgagaaacacggatct
ggacctgtggtgccagagaaggccgtgcggttcagcttcaccatcatgaagatcactatcgcccacagcagccagaacgtgaa
agtgttcgaggaagccaagcctaacagcgagctgtgctgcaagcctctgtgtctgatgctggccgacgagagcgatcacgaga
cactgaccgccattctgagccctctgatcgccgaacgggaagccatgaagtcctccgagctgatgctcgaactcggcggcatcc
tgagaaccttcaagttcatcttccgcggcaccggctacgacgagaagctcgttagagaggtggaaggcctggaagcctctggc
agcgtgtacatctgcaccctgtgtgacgccaccagactggaagctagccagaacctggtgttccacagcatcaccagaagcca
cgccgaaaacctggaaagatacgaagtgtggcggagcaacccctaccacgagagcgtggaagaactgcgggatagagtga
agggcgtgtccgccaagcctttcatcgagacagtgcctagcatcgacgccctgcactgcgatattggcaacgccgccgaattcta
caagatctttcagctggaaatcggcgaggtgtacaagaaccccaacgcctctaaagaggaacggaagcgctggcaggccac
actggataagcacctgagaaagaagatgaatctgaagcccatcatgaggatgaacggcaacttcgcccggaagctgatgacc
aaagaaaccgtggatgccgtgtgcgagctgatcccctctgaggaaagacacgaggccctgcgggaactgatggacctgtacc
tgaagatgaagcccgtgtggcggtctagctgtcctgccaaagagtgccctgagtctctgtgccagtacagcttcaacagccagag
attcgccgagctgctgtccaccaagttcaagtacagatacgagggcaagatcaccaactacttccacaagaccctggctcacgt
gcccgagatcatcgagagagatggctctattggcgcctgggcctctgagggcaatgagtctggcaacaagctgttccggcggtt
ccgcaagatgaacgccagacagagcaagtgctacgagatggaagatgtgctgaagcaccactggctgtacaccagcaagta
cctgcagaaattcatgaacgcccacaacgccctcaagaccagcggctttaccatgaatcctcaggccagcctgggcgaccctct
gggaattgaggatagcctggaatcccaggacagcatggaattctgataa
Right HA
gagtggcacccccacacaccatcctgtgacatctgcaacactgcccgtcggggactcaagaggaagagtcttcagccaaactt
gcagctcagcaaaaaactcaaaactgtgcttgaccaagcaagacaagcccgtcagcgcaagagaagagctcaggcaagg
atcagcagcaaggatgtcatgaagaagatcgccaactgcagtaagatacatcttagtaccaagctccttgcagtggacttccca
gagc
DONOR specific for “g6 M2 ex2 RAG1” gRNA for the exon 2 RAG1 gene replacement
strategy with long right HA
INSERT
gagcacaacaggagatatccagtccatggtcctgtggatggtaaaaccctaggccttttacgaaagaaggaaaagagagcta
cttcctggccggacctcattgccaaggttttccggatcgatgtgaaggcagatgttgactcgatccaccccactgagttctgccata
actgctggagcatcatgcacaggaagtttagcagtgccccatgtgaggtttacttcccgaggaatgtcactatggaatggcaccct
cacacacccagctgcgacatctgcaacacagccagaagaggcctgaagcggaagtccctgcagcctaatctgcagctgagc
aagaaactgaaaaccgtgctggaccaggccagacaggcccggcaaagaaagagaagggcccaagccagaatcagcag
caaggacgtgatgaagaagatcgccaactgcagcaagatccacctgagcaccaaactgctggccgtggacttccctgagcac
ttcgtgaagtccatcagctgccagatctgcgagcacatcctggccgatcctgtggaaacaaactgcaagcacgtgttctgcagag
tgtgcatcctgcggtgcctgaaagtgatgggcagctactgcccctcctgcagatacccttgcttccccaccgatctggaaagccct
gtgaagtccttcctgagcgtgctgaacagcctgatggtcaagtgccccgccaaagaatgcaacgaggaagtgtccctggaaaa
gtacaaccaccacatcagcagccacaaagagtccaaagaaatcttcgtgcacatcaacaaaggcggcagaccccggcagc
atctgctgtctcttacaagacgggcccagaagcaccggctgagagaactgaagctgcaagtgaaggcctttgccgacaaaga
ggaaggcggcgacgtcaagagcgtgtgcatgaccctgtttctgctggccctgagagcccggaatgagcatagacaggccgat
gagctggaagccatcatgcaaggcaaaggcagcggactgcagcctgctgtgtgtctggctatcagagtgaacaccttcctgtcct
gcagccagtaccacaagatgtaccggaccgtgaaggccattaccggcagacagatcttccagcctctgcacgccctgagaaa
cgccgagaaagttctgctgcctggctaccaccacttcgagtggcagcctccactgaagaacgtgtccagcagcaccgacgtgg
gcatcatcgatggactgagcggactgtctagcagcgtggacgactaccccgtggacacaatcgccaagcggttcagatacgac
agcgccctggtgtctgccctgatggacatggaagaggacatcctggaaggcatgcggagccaggacctggacgattacctga
acggccctttcaccgtggtggtcaaagaaagctgtgacggcatgggcgacgtgtccgagaaacacggatctggacctgtggtg
ccagagaaggccgtgcggttcagcttcaccatcatgaagatcactatcgcccacagcagccagaacgtgaaagtgttcgagga
agccaagcctaacagcgagctgtgctgcaagcctctgtgtctgatgctggccgacgagagcgatcacgagacactgaccgcc
attctgagccctctgatcgccgaacgggaagccatgaagtcctccgagctgatgctcgaactcggcggcatcctgagaaccttca
agttcatcttccgcggcaccggctacgacgagaagctcgttagagaggtggaaggcctggaagcctctggcagcgtgtacatct
gcaccctgtgtgacgccaccagactggaagctagccagaacctggtgttccacagcatcaccagaagccacgccgaaaacct
ggaaagatacgaagtgtggcggagcaacccctaccacgagagcgtggaagaactgcgggatagagtgaagggcgtgtccg
ccaagcctttcatcgagacagtgcctagcatcgacgccctgcactgcgatattggcaacgccgccgaattctacaagatctttcag
ctggaaatcggcgaggtgtacaagaaccccaacgcctctaaagaggaacggaagcgctggcaggccacactggataagca
cctgagaaagaagatgaatctgaagcccatcatgaggatgaacggcaacttcgcccggaagctgatgaccaaagaaaccgt
ggatgccgtgtgcgagctgatcccctctgaggaaagacacgaggccctgcgggaactgatggacctgtacctgaagatgaag
cccgtgtggcggtctagctgtcctgccaaagagtgccctgagtctctgtgccagtacagcttcaacagccagagattcgccgagct
gctgtccaccaagttcaagtacagatacgagggcaagatcaccaactacttccacaagaccctggctcacgtgcccgagatcat
cgagagagatggctctattggcgcctgggcctctgagggcaatgagtctggcaacaagctgttccggcggttccgcaagatgaa
cgccagacagagcaagtgctacgagatggaagatgtgctgaagcaccactggctgtacaccagcaagtacctgcagaaattc
atgaacgcccacaacgccctcaagaccagcggctttaccatgaatcctcaggccagcctgggcgatcctttaggcatagagga
ctctctggaaagccaagattcaatggaattttaagtagggcaaccacttatgagttggtttttgcaattgagtttccctctgggttgcatt
gagggcttctcctagcaccctttactgctgtgtatggggcttcaccatccaagaggtggtaggttggagtaagatgctacagatgct
ctcaagtcaggaatagaaactgatgagctgattgcttgaggcttttagtgagttccgaaaagcaacaggaaaaatcagttatctga
aagctcagtaactcagaacaggagtaactgcaggggaccagagatgagcaaagatctgtgtgtgttggggagctgtcatgtaa
atcaaagccaaggttgtcaaagaacagccagtgaggccaggaaagaaattggtcttgtggttttcatttttttcccccttgattgatta
tattttgtattgagatatgataagtgccttctatttcatttttgaataattcttcatttttataattttacatatcttggcttgctatataagattcaa
aagagctttttaaatttttctaataatatcttacatttgtacagcatgatgacctttacaaagtgctctcaatgcatttacccattcgttatat
aaatatgttacatcaggacaactttgagaaaatcagtccttttttatgtttaaattatgtatctattgtaaccttcagagtttaggaggtca
tctgctgtcatggatttttcaataatgaatttagaatacacctgttagctacagttagttattaaatcttctgataatatatgtttacttagcta
tcagaagccaagtatgattctttatttttactttttcatttcaagaaatttagagtttccaaatttagagcttctgcatacagtcttaaagcc
acagaggcttgtaaaaatataggttagcttgatgtctaaaaatatatttcatgtcttactgaaacattttgccagactttctccaaatga
aacctgaatcaatttttctaaatctaggtttcatagagtcctctcctctgcaatgtgttattctttctataatgatcagtttactttcagtggatt
cagaattgtgtagcaggataaccttgtatttttccatccgctaagtttagatggagtccaaacgcagtacagcagaagagttaacat
ttacacagtgctttttaccactgtggaatgttttcacactcatttttccttacaacaattctgaggagtaggtgttgttattatctccatttgat
gggggtttaaatgatttgctcaaagtcatttaggggtaataaatacttggcttggaaatttaacacagtccttttgtctccaaagccctt
cttctttccaccacaaattaatcactatgtttataaggtagtatcagaatttttttaggattcacaactaatcactatagcacatgaccttg
ggattacatttttatggggcaggggtaagcaagtttttaaatcatttgtgtgctctggctcttttgatagaagaaagcaacacaaaag
ctccaaagggccccctaaccctcttgtggctccagttatttggaaactatgatctgcatccttaggaatctgggatttgccagttgctg
gcaatgtagagcaggcatggaattttatatgctagtgagtcataatgatatgttagtgttaattagttttttcttcctttgattttattggccat
aattgctactcttcatacacagtatatcaaagagcttgataatttagttgtcaaaag
Left HA
gagcacaacaggagatatccagtccatggtcctgtggatggtaaaaccctaggccttttacgaaagaaggaaaagagagcta
cttcctggccggacctcattgccaaggttttccggatcgatgtgaaggcagatgttgactcgatccaccccactgagttctgccata
actgctggagcatcatgcacaggaagtttagcagtgccccatgtgaggtttacttcccgaggaatgtcactatg
coRAG1 CDS
gaatggcaccctcacacacccagctgcgacatctgcaacacagccagaagaggcctgaagcggaagtccctgcagcctaat
ctgcagctgagcaagaaactgaaaaccgtgctggaccaggccagacaggcccggcaaagaaagagaagggcccaagcc
agaatcagcagcaaggacgtgatgaagaagatcgccaactgcagcaagatccacctgagcaccaaactgctggccgtgga
cttccctgagcacttcgtgaagtccatcagctgccagatctgcgagcacatcctggccgatcctgtggaaacaaactgcaagcac
gtgttctgcagagtgtgcatcctgcggtgcctgaaagtgatgggcagctactgcccctcctgcagatacccttgcttccccaccgat
ctggaaagccctgtgaagtccttcctgagcgtgctgaacagcctgatggtcaagtgccccgccaaagaatgcaacgaggaagt
gtccctggaaaagtacaaccaccacatcagcagccacaaagagtccaaagaaatcttcgtgcacatcaacaaaggcggcag
accccggcagcatctgctgtctcttacaagacgggcccagaagcaccggctgagagaactgaagctgcaagtgaaggcctttg
ccgacaaagaggaaggcggcgacgtcaagagcgtgtgcatgaccctgtttctgctggccctgagagcccggaatgagcatag
acaggccgatgagctggaagccatcatgcaaggcaaaggcagcggactgcagcctgctgtgtgtctggctatcagagtgaac
accttcctgtcctgcagccagtaccacaagatgtaccggaccgtgaaggccattaccggcagacagatcttccagcctctgcac
gccctgagaaacgccgagaaagttctgctgcctggctaccaccacttcgagtggcagcctccactgaagaacgtgtccagcag
caccgacgtgggcatcatcgatggactgagcggactgtctagcagcgtggacgactaccccgtggacacaatcgccaagcgg
ttcagatacgacagcgccctggtgtctgccctgatggacatggaagaggacatcctggaaggcatgcggagccaggacctgg
acgattacctgaacggccctttcaccgtggtggtcaaagaaagctgtgacggcatgggcgacgtgtccgagaaacacggatct
ggacctgtggtgccagagaaggccgtgcggttcagcttcaccatcatgaagatcactatcgcccacagcagccagaacgtgaa
agtgttcgaggaagccaagcctaacagcgagctgtgctgcaagcctctgtgtctgatgctggccgacgagagcgatcacgaga
cactgaccgccattctgagccctctgatcgccgaacgggaagccatgaagtcctccgagctgatgctcgaactcggcggcatcc
tgagaaccttcaagttcatcttccgcggcaccggctacgacgagaagctcgttagagaggtggaaggcctggaagcctctggc
agcgtgtacatctgcaccctgtgtgacgccaccagactggaagctagccagaacctggtgttccacagcatcaccagaagcca
cgccgaaaacctggaaagatacgaagtgtggcggagcaacccctaccacgagagcgtggaagaactgcgggatagagtga
agggcgtgtccgccaagcctttcatcgagacagtgcctagcatcgacgccctgcactgcgatattggcaacgccgccgaattcta
caagatctttcagctggaaatcggcgaggtgtacaagaaccccaacgcctctaaagaggaacggaagcgctggcaggccac
actggataagcacctgagaaagaagatgaatctgaagcccatcatgaggatgaacggcaacttcgcccggaagctgatgacc
aaagaaaccgtggatgccgtgtgcgagctgatcccctctgaggaaagacacgaggccctgcgggaactgatggacctgtacc
tgaagatgaagcccgtgtggcggtctagctgtcctgccaaagagtgccctgagtctctgtgccagtacagcttcaacagccagag
attcgccgagctgctgtccaccaagttcaagtacagatacgagggcaagatcaccaactacttccacaagaccctggctcacgt
gcccgagatcatcgagagagatggctctattggcgcctgggcctctgagggcaatgagtctggcaacaagctgttccggcggtt
ccgcaagatgaacgccagacagagcaagtgctacgagatggaagatgtgctgaagcaccactggctgtacaccagcaagta
cctgcagaaattcatgaacgcccacaacgccctcaagaccagcggctttaccatgaatcctcaggccagcctgggcgatccttt
Right HA
aggcatagaggactctctggaaagccaagattcaatggaattttaagtagggcaaccacttatgagttggtttttgcaattgagtttc
cctctgggttgcattgagggcttctcctagcaccctttactgctgtgtatggggcttcaccatccaagaggtggtaggttggagtaag
atgctacagatgctctcaagtcaggaatagaaactgatgagctgattgcttgaggcttttagtgagttccgaaaagcaacaggaa
aaatcagttatctgaaagctcagtaactcagaacaggagtaactgcaggggaccagagatgagcaaagatctgtgtgtgttggg
gagctgtcatgtaaatcaaagccaaggttgtcaaagaacagccagtgaggccaggaaagaaattggtcttgtggttttcatttttttc
ccccttgattgattatattttgtattgagatatgataagtgccttctatttcatttttgaataattcttcatttttataattttacatatcttggcttgc
tatataagattcaaaagagctttttaaatttttctaataatatcttacatttgtacagcatgatgacctttacaaagtgctctcaatgcattt
acccattcgttatataaatatgttacatcaggacaactttgagaaaatcagtccttttttatgtttaaattatgtatctattgtaaccttcag
agtttaggaggtcatctgctgtcatggatttttcaataatgaatttagaatacacctgttagctacagttagttattaaatcttctgataat
atatgtttacttagctatcagaagccaagtatgattctttatttttactttttcatttcaagaaatttagagtttccaaatttagagcttctgcat
acagtcttaaagccacagaggcttgtaaaaatataggttagcttgatgtctaaaaatatatttcatgtcttactgaaacattttgccag
actttctccaaatgaaacctgaatcaatttttctaaatctaggtttcatagagtcctctcctctgcaatgtgttattctttctataatgatcag
tttactttcagtggattcagaattgtgtagcaggataaccttgtatttttccatccgctaagtttagatggagtccaaacgcagtacagc
agaagagttaacatttacacagtgctttttaccactgtggaatgttttcacactcatttttccttacaacaattctgaggagtaggtgttgt
tattatctccatttgatgggggtttaaatgatttgctcaaagtcatttaggggtaataaatacttggcttggaaatttaacacagtcctttt
gtctccaaagcccttcttctttccaccacaaattaatcactatgtttataaggtagtatcagaatttttttaggattcacaactaatcacta
tagcacatgaccttgggattacatttttatggggcaggggtaagcaagtttttaaatcatttgtgtgctctggctcttttgatagaagaa
agcaacacaaaagctccaaagggccccctaaccctcttgtggctccagttatttggaaactatgatctgcatccttaggaatctgg
gatttgccagttgctggcaatgtagagcaggcatggaattttatatgctagtgagtcataatgatatgttagtgttaattagttttttcttcc
tttgattttattggccataattgctactcttcatacacagtatatcaaagagcttgataatttagttgtcaaaag
DONOR specific for “g11 exon2 M2/3” gRNA for the exon 2 RAG1 gene targeting
strategy
INSERT
ttcagcacccacatattaaattttcagaatggaaatttaagctgttccgggtgagatcctttgaaaagacacctgaagaagctcaa
aaggaaaagaaggattcctttgaggggaaaccctctctggagcaatctccagcagtcctggacaaggctgatggtcagaagcc
agtcccaactcagccattgttaaaagcccaccctaagttttcaaagaaatttcacgacaacgagaaagcaagaggcaaagcg
atccatcaagccaaccttcgacatctctgccgcatctgtgggaattcttttagagctgatgagcacaacaggagatatccagtccat
ggtcctgtggatggtaaaaccctaggccttttacgaaagaaggaaaagagagctacttcctggccggacctcattgccaaggtttt
ccggatcgatgtgaaggcagatgttgactcgatccaccccactgagttctgccataactgctggagcatcatgcacaggaagttt
agcagtgccccatgtgaggtttacttccccagaaacgtgaccatggaatggcaccctcacacacccagctgcgacatctgcaac
acagccagaagaggcctgaagcggaagtccctgcagcctaatctgcagctgagcaagaaactgaaaaccgtgctggacca
ggccagacaggcccggcaaagaaagagaagggcccaagccagaatcagcagcaaggacgtgatgaagaagatcgcca
actgcagcaagatccacctgagcaccaaactgctggccgtggacttccctgagcacttcgtgaagtccatcagctgccagatctg
cgagcacatcctggccgatcctgtggaaacaaactgcaagcacgtgttctgcagagtgtgcatcctgcggtgcctgaaagtgat
gggcagctactgcccctcctgcagatacccttgcttccccaccgatctggaaagccctgtgaagtccttcctgagcgtgctgaaca
gcctgatggtcaagtgccccgccaaagaatgcaacgaggaagtgtccctggaaaagtacaaccaccacatcagcagccaca
aagagtccaaagaaatcttcgtgcacatcaacaaaggcggcagaccccggcagcatctgctgtctcttacaagacgggccca
gaagcaccggctgagagaactgaagctgcaagtgaaggcctttgccgacaaagaggaaggcggcgacgtcaagagcgtgt
gcatgaccctgtttctgctggccctgagagcccggaatgagcatagacaggccgatgagctggaagccatcatgcaaggcaa
aggcagcggactgcagcctgctgtgtgtctggctatcagagtgaacaccttcctgtcctgcagccagtaccacaagatgtaccgg
accgtgaaggccattaccggcagacagatcttccagcctctgcacgccctgagaaacgccgagaaagttctgctgcctggctac
caccacttcgagtggcagcctccactgaagaacgtgtccagcagcaccgacgtgggcatcatcgatggactgagcggactgtc
tagcagcgtggacgactaccccgtggacacaatcgccaagcggttcagatacgacagcgccctggtgtctgccctgatggaca
tggaagaggacatcctggaaggcatgcggagccaggacctggacgattacctgaacggccctttcaccgtggtggtcaaaga
aagctgtgacggcatgggcgacgtgtccgagaaacacggatctggacctgtggtgccagagaaggccgtgcggttcagcttca
ccatcatgaagatcactatcgcccacagcagccagaacgtgaaagtgttcgaggaagccaagcctaacagcgagctgtgctg
caagcctctgtgtctgatgctggccgacgagagcgatcacgagacactgaccgccattctgagccctctgatcgccgaacggga
agccatgaagtcctccgagctgatgctcgaactcggcggcatcctgagaaccttcaagttcatcttccgcggcaccggctacgac
gagaagctcgttagagaggtggaaggcctggaagcctctggcagcgtgtacatctgcaccctgtgtgacgccaccagactgga
agctagccagaacctggtgttccacagcatcaccagaagccacgccgaaaacctggaaagatacgaagtgtggcggagca
acccctaccacgagagcgtggaagaactgcgggatagagtgaagggcgtgtccgccaagcctttcatcgagacagtgcctag
catcgacgccctgcactgcgatattggcaacgccgccgaattctacaagatctttcagctggaaatcggcgaggtctacaagaa
ccccaacgcctctaaagaggaacggaagcgctggcaggccacactggataagcacctgagaaagaagatgaatctgaagc
ccatcatgaggatgaacggcaacttcgcccggaagctgatgaccaaagaaaccgtggatgccgtgtgcgagctgatcccctct
gaggaaagacacgaggccctgcgggaactgatggacctgtacctgaagatgaagcccgtgtggcggtctagctgtcctgcca
aagagtgccctgagtctctgtgccagtacagcttcaacagccagagattcgccgagctgctgtccaccaagttcaagtacagata
cgagggcaagatcaccaactacttccacaagaccctggctcacgtgcccgagatcatcgagagagatggctctattggcgcct
gggcctctgagggcaatgagtctggcaacaagctgttccggcggttccgcaagatgaacgccagacagagcaagtgctacga
gatggaagatgtgctgaagcaccactggctgtacaccagcaagtacctgcagaaattcatgaacgcccacaacgccctcaag
accagcggctttaccatgaatcctcaggccagcctgggcgatcctctgggaattgaggatagcctggaatcccaggacagcatg
gaattctgataaccgaggaacgtgaccatggagtggcacccccacacaccatcctgtgacatctgcaacactgcccgtcgggg
actcaagaggaagagtcttcagccaaacttgcagctcagcaaaaaactcaaaactgtgcttgaccaagcaagacaagcccgt
cagcgcaagagaagagctcaggcaaggatcagcagcaaggatgtcatgaagaagatcgccaactgcagtaagatacatctt
agtaccaagctccttgcagtggacttcccagagcactttgtgaaatccatctcctgccagatctgtgaacacattctggctgaccctg
tggagaccaactgtaagcatgtcttttgccgggtctgcattctcagatgcctcaaagtcatgggcagctattgtccctcttgccgatat
ccatgcttccctactgacctggagagtccagtgaagtcctttctgagcgtcttgaattccctgatggtgaaatgtccagcaaaagagt
g
Left HA
ttcagcacccacatattaaattttcagaatggaaatttaagctgttccgggtgagatcctttgaaaagacacctgaagaagctcaa
aaggaaaagaaggattcctttgaggggaaaccctctctggagcaatctccagcagtcctg gacaaggctgatggtcagaagcc
agtcccaactcagccattgttaaaagcccaccctaagttttcaaagaaatttcacgacaacgagaaagcaagaggcaaagcg
atccatcaagccaaccttcgacatctctgccgcatctgtgggaattcttttagagctgatgagcacaacaggagatatccagtccat
ggtcctgtggatggtaaaaccctaggccttttacgaaagaaggaaaagagagctacttcctggccggacctcattgccaaggtttt
ccggatcgatgtgaaggcagatgttgactcgatccaccccactgagttctgccataactgctggagcatcatgcacaggaagttt
agcagtgccccatgtgaggtttacttc
coRAG1 CDS
cccagaaacgtgaccatggaatggcaccctcacacacccagctgcgacatctgcaacacagccagaagaggcctgaagcg
gaagtccctgcagcctaatctgcagctgagcaagaaactgaaaaccgtgctggaccaggccagacaggcccggcaaagaa
agagaagggcccaagccagaatcagcagcaaggacgtgatgaagaagatcgccaactgcagcaagatccacctgagcac
caaactgctggccgtggacttccctgagcacttcgtgaagtccatcagctgccagatctgcgagcacatcctggccgatcctgtgg
aaacaaactgcaagcacgtgttctgcagagtgtgcatcctgcggtgcctgaaagtgatgggcagctactgcccctcctgcagata
cccttgcttccccaccgatctggaaagccctgtgaagtccttcctgagcgtgctgaacagcctgatggtcaagtgccccgccaaa
gaatgcaacgaggaagtgtccctggaaaagtacaaccaccacatcagcagccacaaagagtccaaagaaatcttcgtgcac
atcaacaaaggcggcagaccccggcagcatctgctgtctcttacaagacgggcccagaagcaccggctgagagaactgaag
ctgcaagtgaaggcctttgccgacaaagaggaaggcggcgacgtcaagagcgtgtgcatgaccctgtttctgctggccctgag
agcccggaatgagcatagacaggccgatgagctggaagccatcatgcaaggcaaaggcagcggactgcagcctgctgtgtg
tctggctatcagagtgaacaccttcctgtcctgcagccagtaccacaagatgtaccggaccgtgaaggccattaccggcagaca
gatcttccagcctctgcacgccctgagaaacgccgagaaagttctgctgcctggctaccaccacttcgagtggcagcctccactg
aagaacgtgtccagcagcaccgacgtgggcatcatcgatggactgagcggactgtctagcagcgtggacgactaccccgtgg
acacaatcgccaagcggttcagatacgacagcgccctggtgtctgccctgatggacatggaagaggacatcctggaaggcat
gcggagccaggacctggacgattacctgaacggccctttcaccgtggtggtcaaagaaagctgtgacggcatgggcgacgtgt
ccgagaaacacggatctggacctgtggtgccagagaaggccgtgcggttcagcttcaccatcatgaagatcactatcgcccac
agcagccagaacgtgaaagtgttcgaggaagccaagcctaacagcgagctgtgctgcaagcctctgtgtctgatgctggccga
cgagagcgatcacgagacactgaccgccattctgagccctctgatcgccgaacgggaagccatgaagtcctccgagctgatgc
tcgaactcggcggcatcctgagaaccttcaagttcatcttccgcggcaccggctacgacgagaagctcgttagagaggtggaag
gcctggaagcctctggcagcgtgtacatctgcaccctgtgtgacgccaccagactggaagctagccagaacctggtgttccaca
gcatcaccagaagccacgccgaaaacctggaaagatacgaagtgtggcggagcaacccctaccacgagagcgtggaaga
actgcgggatagagtgaagggcgtgtccgccaagcctttcatcgagacagtgcctagcatcgacgccctgcactgcgatattgg
caacgccgccgaattctacaagatctttcagctggaaatcggcgaggtctacaagaaccccaacgcctctaaagaggaacgg
aagcgctggcaggccacactggataagcacctgagaaagaagatgaatctgaagcccatcatgaggatgaacggcaacttc
gcccggaagctgatgaccaaagaaaccgtggatgccgtgtgcgagctgatcccctctgaggaaagacacgaggccctgcgg
gaactgatggacctgtacctgaagatgaagcccgtgtggcggtctagctgtcctgccaaagagtgccctgagtctctgtgccagt
acagcttcaacagccagagattcgccgagctgctgtccaccaagttcaagtacagatacgagggcaagatcaccaactacttc
cacaagaccctggctcacgtgcccgagatcatcgagagagatggctctattggcgcctgggcctctgagggcaatgagtctggc
aacaagctgttccggcggttccgcaagatgaacgccagacagagcaagtgctacgagatggaagatgtgctgaagcaccact
ggctgtacaccagcaagtacctgcagaaattcatgaacgcccacaacgccctcaagaccagcggctttaccatgaatcctcag
gccagcctgggcgatcctctgggaattgaggatagcctggaatcccaggacagcatggaattctga
Right HA
ccgaggaacgtgaccatggagtggcacccccacacaccatcctgtgacatctgcaacactgcccgtcggggactcaagagga
agagtcttcagccaaacttgcagctcagcaaaaaactcaaaactgtgcttgaccaagcaagacaagcccgtcagcgcaagag
aagagctcaggcaaggatcagcagcaaggatgtcatgaagaagatcgccaactgcagtaagatacatcttagtaccaagctc
cttgcagtggacttcccagagcactttgtgaaatccatctcctgccagatctgtgaacacattctggctgaccctgtggagaccaac
tgtaagcatgtcttttgccgggtctgcattctcagatgcctcaaagtcatgggcagctattgtccctcttgccgatatccatgcttcccta
ctgacctggagagtccagtgaagtcctttctgagcgtcttgaattccctgatggtgaaatgtccagcaaaagagtg
DONOR specific for “g11 exon2 M2/3” gRNA for the exon 2 RAG1 gene replacement
strategy
INSERT
ttcagcacccacatattaaattttcagaatggaaatttaagctgttccgggtgagatcctttgaaaagacacctgaagaagctcaa
aaggaaaagaaggattcctttgaggggaaaccctctctggagcaatctccagcagtcctggacaaggctgatggtcagaagcc
agtcccaactcagccattgttaaaagcccaccctaagttttcaaagaaatttcacgacaacgagaaagcaagaggcaaagcg
atccatcaagccaaccttcgacatctctgccgcatctgtgggaattcttttagagctgatgagcacaacaggagatatccagtccat
ggtcctgtggatggtaaaaccctaggccttttacgaaagaaggaaaagagagctacttcctggccggacctcattgccaaggtttt
ccggatcgatgtgaaggcagatgttgactcgatccaccccactgagttctgccataactgctggagcatcatgcacaggaagttt
agcagtgccccatgtgaggtttacttccccagaaacgtgaccatggaatggcaccctcacacacccagctgcgacatctgcaac
acagccagaagaggcctgaagcggaagtccctgcagcctaatctgcagctgagcaagaaactgaaaaccgtgctggacca
ggccagacaggcccggcaaagaaagagaagggcccaagccagaatcagcagcaaggacgtgatgaagaagatcgcca
actgcagcaagatccacctgagcaccaaactgctggccgtggacttccctgagcacttcgtgaagtccatcagctgccagatctg
cgagcacatcctggccgatcctgtggaaacaaactgcaagcacgtgttctgcagagtgtgcatcctgcggtgcctgaaagtgat
gggcagctactgcccctcctgcagatacccttgcttccccaccgatctggaaagccctgtgaagtccttcctgagcgtgctgaaca
gcctgatggtcaagtgccccgccaaagaatgcaacgaggaagtgtccctggaaaagtacaaccaccacatcagcagccaca
aagagtccaaagaaatcttcgtgcacatcaacaaaggcggcagaccccggcagcatctgctgtctcttacaagacgggccca
gaagcaccggctgagagaactgaagctgcaagtgaaggcctttgccgacaaagaggaaggcggcgacgtcaagagcgtgt
gcatgaccctgtttctgctggccctgagagcccggaatgagcatagacaggccgatgagctggaagccatcatgcaaggcaa
aggcagcggactgcagcctgctgtgtgtctggctatcagagtgaacaccttcctgtcctgcagccagtaccacaagatgtaccgg
accgtgaaggccattaccggcagacagatcttccagcctctgcacgccctgagaaacgccgagaaagttctgctgcctggctac
caccacttcgagtggcagcctccactgaagaacgtgtccagcagcaccgacgtgggcatcatcgatggactgagcggactgtc
tagcagcgtggacgactaccccgtggacacaatcgccaagcggttcagatacgacagcgccctggtgtctgccctgatggaca
tggaagaggacatcctggaaggcatgcggagccaggacctggacgattacctgaacggccctttcaccgtggtggtcaaaga
aagctgtgacggcatgggcgacgtgtccgagaaacacggatctggacctgtggtgccagagaaggccgtgcggttcagcttca
ccatcatgaagatcactatcgcccacagcagccagaacgtgaaagtgttcgaggaagccaagcctaacagcgagctgtgctg
caagcctctgtgtctgatgctggccgacgagagcgatcacgagacactgaccgccattctgagccctctgatcgccgaacggga
agccatgaagtcctccgagctgatgctcgaactcggcggcatcctgagaaccttcaagttcatcttccgcggcaccggctacgac
gagaagctcgttagagaggtggaaggcctggaagcctctggcagcgtgtacatctgcaccctgtgtgacgccaccagactgga
agctagccagaacctggtgttccacagcatcaccagaagccacgccgaaaacctggaaagatacgaagtgtggcggagca
acccctaccacgagagcgtggaagaactgcgggatagagtgaagggcgtgtccgccaagcctttcatcgagacagtgcctag
catcgacgccctgcactgcgatattggcaacgccgccgaattctacaagatctttcagctggaaatcggcgaggtctacaagaa
ccccaacgcctctaaagaggaacggaagcgctggcaggccacactggataagcacctgagaaagaagatgaatctgaagc
ccatcatgaggatgaacggcaacttcgcccggaagctgatgaccaaagaaaccgtggatgccgtgtgcgagctgatcccctct
gaggaaagacacgaggccctgcgggaactgatggacctgtacctgaagatgaagcccgtgtggcggtctagctgtcctgcca
aagagtgccctgagtctctgtgccagtacagcttcaacagccagagattcgccgagctgctgtccaccaagttcaagtacagata
cgagggcaagatcaccaactacttccacaagaccctggctcacgtgcccgagatcatcgagagagatggctctattggcgcct
gggcctctgagggcaatgagtctggcaacaagctgttccggcggttccgcaagatgaacgccagacagagcaagtgctacga
gatggaagatgtgctgaagcaccactggctgtacaccagcaagtacctgcagaaattcatgaacgcccacaacgccctcaag
accagcggctttaccatgaatcctcaggccagcctgggcgatcctttaggcatagaggactctctggaaagccaagattcaatgg
aattttaagtagggcaaccacttatgagttggtttttgcaattgagtttccctctgggttgcattgagggcttctcctagcaccctttactg
ctgtgtatggggcttcaccatccaagaggtggtaggttggagtaagatgctacagatgctctcaagtcaggaatagaaactgatg
agctgattgcttgaggcttttagtgagttccgaaaagcaacaggaaaaatcagttatctgaaagctcagtaactcagaacaggag
taactgcaggggaccagagatgagcaaagatctgtgtgtgttggggagctgtcatgtaaatcaaagccaaggttgtcaaagaac
agccagtgaggccaggaaagaaattggtcttgtggttttcatttttttcccccttgattgattatattttgtattgagatatgataagtgcctt
ctatttcatttttgaataattcttcatttttataattttacatatcttggcttgctatataagattcaaaagagctttttaaatttttctaataatatc
ttacatttgtacagcatgatgacctttacaaagtgctctcaatgcatttacccattcgttatataaatatgttacatcaggacaactttgag
aaaatcagtccttttttatgtttaaattatgtatctattgtaaccttcagagtttaggaggtcatctgctgtcatggatttttcaataatgaatt
tagaatacacctgttagctacagttagttattaaatcttctgataatatatgtttacttagctatcagaagccaagtatgattctttattttta
ctttttcatttcaagaaatttagagtttccaaatttagagcttctgcatacagtcttaaagccacagaggcttgtaaaaatataggttag
cttgatgtctaaaaatatatttcatgtcttactgaaacattttgccagactttctccaaatgaaacctgaatcaatttttctaaatctaggttt
catagagtcctctcctctgcaatgtgttattctttctataatgatcagtttactttcagtggattcagaattgtgtagcaggataaccttgtat
ttttccatccgctaagtttagatggagtccaaacgcagtacagcagaagagtt
Left HA
ttcagcacccacatattaaattttcagaatggaaatttaagctgttccgggtgagatcctttgaaaagacacctgaagaagctcaa
aaggaaaagaaggattcctttgaggggaaaccctctctggagcaatctccagcagtcctggacaaggctgatggtcagaagcc
agtcccaactcagccattgttaaaagcccaccctaagttttcaaagaaatttcacgacaacgagaaagcaagaggcaaagcg
atccatcaagccaaccttcgacatctctgccgcatctgtgggaattcttttagagctgatgagcacaacaggagatatccagtccat
ggtcctgtggatggtaaaaccctaggccttttacgaaagaaggaaaagagagctacttcctggccggacctcattgccaaggtttt
ccggatcgatgtgaaggcagatgttgactcgatccaccccactgagttctgccataactgctggagcatcatgcacaggaagttt
agcagtgccccatgtgaggtttacttc
coRAG1 CDS
cccagaaacgtgaccatggaatggcaccctcacacacccagctgcgacatctgcaacacagccagaagaggcctgaagcg
gaagtccctgcagcctaatctgcagctgagcaagaaactgaaaaccgtgctggaccaggccagacaggcccggcaaagaa
agagaagggcccaagccagaatcagcagcaaggacgtgatgaagaagatcgccaactgcagcaagatccacctgagcac
caaactgctggccgtggacttccctgagcacttcgtgaagtccatcagctgccagatctgcgagcacatcctggccgatcctgtgg
aaacaaactgcaagcacgtgttctgcagagtgtgcatcctgcggtgcctgaaagtgatgggcagctactgcccctcctgcagata
cccttgcttccccaccgatctggaaagccctgtgaagtccttcctgagcgtgctgaacagcctgatggtcaagtgccccgccaaa
gaatgcaacgaggaagtgtccctggaaaagtacaaccaccacatcagcagccacaaagagtccaaagaaatcttcgtgcac
atcaacaaaggcggcagaccccggcagcatctgctgtctcttacaagacgggcccagaagcaccggctgagagaactgaag
ctgcaagtgaaggcctttgccgacaaagaggaaggcggcgacgtcaagagcgtgtgcatgaccctgtttctgctggccctgag
agcccggaatgagcatagacaggccgatgagctggaagccatcatgcaaggcaaaggcagcggactgcagcctgctgtgtg
tctggctatcagagtgaacaccttcctgtcctgcagccagtaccacaagatgtaccggaccgtgaaggccattaccggcagaca
gatcttccagcctctgcacgccctgagaaacgccgagaaagttctgctgcctggctaccaccacttcgagtggcagcctccactg
aagaacgtgtccagcagcaccgacgtgggcatcatcgatggactgagcggactgtctagcagcgtggacgactaccccgtgg
acacaatcgccaagcggttcagatacgacagcgccctggtgtctgccctgatggacatggaagaggacatcctggaaggcat
gcggagccaggacctggacgattacctgaacggccctttcaccgtggtggtcaaagaaagctgtgacggcatgggcgacgtgt
ccgagaaacacggatctggacctgtggtgccagagaaggccgtgcggttcagcttcaccatcatgaagatcactatcgcccac
agcagccagaacgtgaaagtgttcgaggaagccaagcctaacagcgagctgtgctgcaagcctctgtgtctgatgctggccga
cgagagcgatcacgagacactgaccgccattctgagccctctgatcgccgaacgggaagccatgaagtcctccgagctgatgc
tcgaactcggcggcatcctgagaaccttcaagttcatcttccgcggcaccggctacgacgagaagctcgttagagaggtggaag
gcctggaagcctctggcagcgtgtacatctgcaccctgtgtgacgccaccagactggaagctagccagaacctggtgttccaca
gcatcaccagaagccacgccgaaaacctggaaagatacgaagtgtggcggagcaacccctaccacgagagcgtggaaga
actgcgggatagagtgaagggcgtgtccgccaagcctttcatcgagacagtgcctagcatcgacgccctgcactgcgatattgg
caacgccgccgaattctacaagatctttcagctggaaatcggcgaggtctacaagaaccccaacgcctctaaagaggaacgg
aagcgctggcaggccacactggataagcacctgagaaagaagatgaatctgaagcccatcatgaggatgaacggcaacttc
gcccggaagctgatgaccaaagaaaccgtggatgccgtgtgcgagctgatcccctctgaggaaagacacgaggccctgcgg
gaactgatggacctgtacctgaagatgaagcccgtgtggcggtctagctgtcctgccaaagagtgccctgagtctctgtgccagt
acagcttcaacagccagagattcgccgagctgctgtccaccaagttcaagtacagatacgagggcaagatcaccaactacttc
cacaagaccctggctcacgtgcccgagatcatcgagagagatggctctattggcgcctgggcctctgagggcaatgagtctggc
aacaagctgttccggcggttccgcaagatgaacgccagacagagcaagtgctacgagatggaagatgtgctgaagcaccact
ggctgtacaccagcaagtacctgcagaaattcatgaacgcccacaacgccctcaagaccagcggctttaccatgaatcctcag
gccagcctgggcgatccttt
Right HA
aggcatagaggactctctggaaagccaagattcaatggaattttaagtagggcaaccacttatgagttggtttttgcaattgagtttc
cctctgggttgcattgagggcttctcctagcaccctttactgctgtgtatggggcttcaccatccaagaggtggtaggttggagtaag
atgctacagatgctctcaagtcaggaatagaaactgatgagctgattgcttgaggcttttagtgagttccgaaaagcaacaggaa
aaatcagttatctgaaagctcagtaactcagaacaggagtaactgcaggggaccagagatgagcaaagatctgtgtgtgttggg
gagctgtcatgtaaatcaaagccaaggttgtcaaagaacagccagtgaggccaggaaagaaattggtcttgtggttttcatttttttc
ccccttgattgattatattttgtattgagatatgataagtgccttctatttcatttttgaataattcttcatttttataattttacatatcttggc
ttgctatataagattcaaaagagctttttaaatttttctaataatatcttacatttgtacagcatgatgacctttacaaagtgctctcaatgcattt
acccattcgttatataaatatgttacatcaggacaactttgagaaaatcagtccttttttatgtttaaattatgtatctattgtaaccttcag
agtttaggaggtcatctgctgtcatggatttttcaataatgaatttagaatacacctgttagctacagttagttattaaatcttctgataat
atatgtttacttagctatcagaagccaagtatgattctttatttttactttttcatttcaagaaatttagagtttccaaatttagagcttctgcat
acagtcttaaagccacagaggcttgtaaaaatataggttagcttgatgtctaaaaatatatttcatgtcttactgaaacattttgccag
actttctccaaatgaaacctgaatcaatttttctaaatctaggtttcatagagtcctctcctctgcaatgtgttattctttctataatgatcag
tttactttcagtggattcagaattgtgtagcaggataaccttgtatttttccatccgctaagtttagatggagtccaaacgcagtacagc
agaagagtt
DONOR specific for “g13 exon2 M2/3” gRNA for the exon 2 RAG1 gene targeting
strategy
INSERT
ttcagcacccacatattaaattttcagaatggaaatttaagctgttccgggtgagatcctttgaaaagacacctgaagaagctcaa
aaggaaaagaaggattcctttgaggggaaaccctctctggagcaatctccagcagtcctggacaaggctgatggtcagaagcc
agtcccaactcagccattgttaaaagcccaccctaagttttcaaagaaatttcacgacaacgagaaagcaagaggcaaagcg
atccatcaagccaaccttcgacatctctgccgcatctgtgggaattcttttagagctgatgagcacaacaggagatatccagtccat
ggtcctgtggatggtaaaaccctaggccttttacgaaagaaggaaaagagagctacttcctggccggacctcattgccaaggtttt
ccggatcgatgtgaaggcagatgttgactcgatccaccccactgagttctgccataactgctggagcatcatgcacaggaagttt
agcagtgcaccatgcgaagtgtacttccccagaaacgtgaccatggaatggcaccctcacacacccagctgcgacatctgcaa
cacagccagaagaggcctgaagcggaagtccctgcagcctaatctgcagctgagcaagaaactgaaaaccgtgctggacca
ggccagacaggcccggcaaagaaagagaagggcccaagccagaatcagcagcaaggacgtgatgaagaagatcgcca
actgcagcaagatccacctgagcaccaaactgctggccgtggacttccctgagcacttcgtgaagtccatcagctgccagatctg
cgagcacatcctggccgatcctgtggaaacaaactgcaagcacgtgttctgcagagtgtgcatcctgcggtgcctgaaagtgat
gggcagctactgcccctcctgcagatacccttgcttccccaccgatctggaaagccctgtgaagtccttcctgagcgtgctgaaca
gcctgatggtcaagtgccccgccaaagaatgcaacgaggaagtgtccctggaaaagtacaaccaccacatcagcagccaca
aagagtccaaagaaatcttcgtgcacatcaacaaaggcggcagaccccggcagcatctgctgtctcttacaagacgggccca
gaagcaccggctgagagaactgaagctgcaagtgaaggcctttgccgacaaagaggaaggcggcgacgtcaagagcgtgt
gcatgaccctgtttctgctggccctgagagcccggaatgagcatagacaggccgatgagctggaagccatcatgcaaggcaa
aggcagcggactgcagcctgctgtgtgtctggctatcagagtgaacaccttcctgtcctgcagccagtaccacaagatgtaccgg
accgtgaaggccattaccggcagacagatcttccagcctctgcacgccctgagaaacgccgagaaagttctgctgcctggctac
caccacttcgagtggcagcctccactgaagaacgtgtccagcagcaccgacgtgggcatcatcgatggactgagcggactgtc
tagcagcgtggacgactaccccgtggacacaatcgccaagcggttcagatacgacagcgccctggtgtctgccctgatggaca
tggaagaggacatcctggaaggcatgcggagccaggacctggacgattacctgaacggccctttcaccgtggtggtcaaaga
aagctgtgacggcatgggcgacgtgtccgagaaacacggatctggacctgtggtgccagagaaggccgtgcggttcagcttca
ccatcatgaagatcactatcgcccacagcagccagaacgtgaaagtgttcgaggaagccaagcctaacagcgagctgtgctg
caagcctctgtgtctgatgctggccgacgagagcgatcacgagacactgaccgccattctgagccctctgatcgccgaacggga
agccatgaagtcctccgagctgatgctcgaactcggcggcatcctgagaaccttcaagttcatcttccgcggcaccggctacgac
gagaagctcgttagagaggtggaaggcctggaagcctctggcagcgtgtacatctgcaccctgtgtgacgccaccagactgga
agctagccagaacctggtgttccacagcatcaccagaagccacgccgaaaacctggaaagatacgaagtgtggcggagca
acccctaccacgagagcgtggaagaactgcgggatagagtgaagggcgtgtccgccaagcctttcatcgagacagtgcctag
catcgacgccctgcactgcgatattggcaacgccgccgaattctacaagatctttcagctggaaatcggcgaggtctacaagaa
ccccaacgcctctaaagaggaacggaagcgctggcaggccacactggataagcacctgagaaagaagatgaatctgaagc
ccatcatgaggatgaacggcaacttcgcccggaagctgatgaccaaagaaaccgtggatgccgtgtgcgagctgatcccctct
gaggaaagacacgaggccctgcgggaactgatggacctgtacctgaagatgaagcccgtgtggcggtctagctgtcctgcca
aagagtgccctgagtctctgtgccagtacagcttcaacagccagagattcgccgagctgctgtccaccaagttcaagtacagata
cgagggcaagatcaccaactacttccacaagaccctggctcacgtgcccgagatcatcgagagagatggctctattggcgcct
gggcctctgagggcaatgagtctggcaacaagctgttccggcggttccgcaagatgaacgccagacagagcaagtgctacga
gatggaagatgtgctgaagcaccactggctgtacaccagcaagtacctgcagaaattcatgaacgcccacaacgccctcaag
accagcggctttaccatgaatcctcaggccagcctgggcgatcctctgggaattgaggatagcctggaatcccaggacagcatg
gaattctgataagtgaggtttacttcccgaggaacgtgaccatggagtggcacccccacacaccatcctgtgacatctgcaacac
tgcccgtcggggactcaagaggaagagtcttcagccaaacttgcagctcagcaaaaaactcaaaactgtgcttgaccaagca
agacaagcccgtcagcgcaagagaagagctcaggcaaggatcagcagcaaggatgtcatgaagaagatcgccaactgca
gtaagatacatcttagtaccaagctccttgcagtggacttcccagagcactttgtgaaatccatctcctgccagatctgtgaacacat
tctggctgaccctgtggagaccaactgtaagcatgtcttttgccgggtctgcattctcagatgcctcaaagtcatgggcagctattgtc
cctcttgccgatatccatgcttccctactgacctggagagtccagtgaagtcctttctgagcgtcttgaattccctgatggtgaaatgt
Left HA
ttcagcacccacatattaaattttcagaatggaaatttaagctgttccgggtgagatcctttgaaaagacacctgaagaagctcaa
aaggaaaagaaggattcctttgaggggaaaccctctctggagcaatctccagcagtcctggacaaggctgatggtcagaagcc
agtcccaactcagccattgttaaaagcccaccctaagttttcaaagaaatttcacgacaacgagaaagcaagaggcaaagcg
atccatcaagccaaccttcgacatctctgccgcatctgtgggaattcttttagagctgatgagcacaacaggagatatccagtccat
ggtcctgtggatggtaaaaccctaggccttttacgaaagaaggaaaagagagctacttcctggccggacctcattgccaaggtttt
ccggatcgatgtgaaggcagatgttgactcgatccaccccactgagttctgccataactgctggagcatcatgcacaggaagttt
agcagtgcaccat
coRAG1 CDS
gcgaagtgtacttccccagaaacgtgaccatggaatggcaccctcacacacccagctgcgacatctgcaacacagccagaag
aggcctgaagcggaagtccctgcagcctaatctgcagctgagcaagaaactgaaaaccgtgctggaccaggccagacaggc
ccggcaaagaaagagaagggcccaagccagaatcagcagcaaggacgtgatgaagaagatcgccaactgcagcaagat
ccacctgagcaccaaactgctggccgtggacttccctgagcacttcgtgaagtccatcagctgccagatctgcgagcacatcctg
gccgatcctgtggaaacaaactgcaagcacgtgttctgcagagtgtgcatcctgcggtgcctgaaagtgatgggcagctactgc
ccctcctgcagatacccttgcttccccaccgatctggaaagccctgtgaagtccttcctgagcgtgctgaacagcctgatggtcaa
gtgccccgccaaagaatgcaacgaggaagtgtccctggaaaagtacaaccaccacatcagcagccacaaagagtccaaag
aaatcttcgtgcacatcaacaaaggcggcagaccccggcagcatctgctgtctcttacaagacgggcccagaagcaccggctg
agagaactgaagctgcaagtgaaggcctttgccgacaaagaggaaggcggcgacgtcaagagcgtgtgcatgaccctgtttct
gctggccctgagagcccggaatgagcatagacaggccgatgagctggaagccatcatgcaaggcaaaggcagcggactgc
agcctgctgtgtgtctggctatcagagtgaacaccttcctgtcctgcagccagtaccacaagatgtaccggaccgtgaaggccatt
accggcagacagatcttccagcctctgcacgccctgagaaacgccgagaaagttctgctgcctggctaccaccacttcgagtgg
cagcctccactgaagaacgtgtccagcagcaccgacgtgggcatcatcgatggactgagcggactgtctagcagcgtggacg
actaccccgtggacacaatcgccaagcggttcagatacgacagcgccctggtgtctgccctgatggacatggaagaggacatc
ctggaaggcatgcggagccaggacctggacgattacctgaacggccctttcaccgtggtggtcaaagaaagctgtgacggcat
gggcgacgtgtccgagaaacacggatctggacctgtggtgccagagaaggccgtgcggttcagcttcaccatcatgaagatca
ctatcgcccacagcagccagaacgtgaaagtgttcgaggaagccaagcctaacagcgagctgtgctgcaagcctctgtgtctg
atgctggccgacgagagcgatcacgagacactgaccgccattctgagccctctgatcgccgaacgggaagccatgaagtcctc
cgagctgatgctcgaactcggcggcatcctgagaaccttcaagttcatcttccgcggcaccggctacgacgagaagctcgttag
agaggtggaaggcctggaagcctctggcagcgtgtacatctgcaccctgtgtgacgccaccagactggaagctagccagaac
ctggtgttccacagcatcaccagaagccacgccgaaaacctggaaagatacgaagtgtggcggagcaacccctaccacgag
agcgtggaagaactgcgggatagagtgaagggcgtgtccgccaagcctttcatcgagacagtgcctagcatcgacgccctgc
actgcgatattggcaacgccgccgaattctacaagatctttcagctggaaatcggcgaggtctacaagaaccccaacgcctcta
aagaggaacggaagcgctggcaggccacactggataagcacctgagaaagaagatgaatctgaagcccatcatgaggatg
aacggcaacttcgcccggaagctgatgaccaaagaaaccgtggatgccgtgtgcgagctgatcccctctgaggaaagacacg
aggccctgcgggaactgatggacctgtacctgaagatgaagcccgtgtggcggtctagctgtcctgccaaagagtgccctgagt
ctctgtgccagtacagcttcaacagccagagattcgccgagctgctgtccaccaagttcaagtacagatacgagggcaagatca
ccaactacttccacaagaccctggctcacgtgcccgagatcatcgagagagatggctctattggcgcctgggcctctgagggca
atgagtctggcaacaagctgttccggcggttccgcaagatgaacgccagacagagcaagtgctacgagatggaagatgtgctg
aagcaccactggctgtacaccagcaagtacctgcagaaattcatgaacgcccacaacgccctcaagaccagcggctttaccat
gaatcctcaggccagcctgggcgatcctctgggaattgaggatagcctggaatcccaggacagcatggaattctga
Right HA
gtgaggtttacttcccgaggaacgtgaccatggagtggcacccccacacaccatcctgtgacatctgcaacactgcccgtcggg
gactcaagaggaagagtcttcagccaaacttgcagctcagcaaaaaactcaaaactgtgcttgaccaagcaagacaagccc
gtcagcgcaagagaagagctcaggcaaggatcagcagcaaggatgtcatgaagaagatcgccaactgcagtaagatacat
cttagtaccaagctccttgcagtggacttcccagagcactttgtgaaatccatctcctgccagatctgtgaacacattctggctgacc
ctgtggagaccaactgtaagcatgtcttttgccgggtctgcattctcagatgcctcaaagtcatgggcagctattgtccctcttgccga
tatccatgcttccctactgacctggagagtccagtgaagtcctttctgagcgtcttgaattccctgatggtgaaatgt
DONOR specific for “g13 exon2 M2/3”, “g7 exon2 M2/3”, “g10 exon2 M2/3” gRNAs for
the exon 2 RAG1 gene replacement strategy
INSERT
ttcagcacccacatattaaattttcagaatggaaatttaagctgttccgggtgagatcctttgaaaagacacctgaagaagctcaa
aaggaaaagaaggattcctttgaggggaaaccctctctggagcaatctccagcagtcctggacaaggctgatggtcagaagcc
agtcccaactcagccattgttaaaagcccaccctaagttttcaaagaaatttcacgacaacgagaaagcaagaggcaaagcg
atccatcaagccaaccttcgacatctctgccgcatctgtgggaattcttttagagctgatgagcacaacaggagatatccagtccat
ggtcctgtggatggtaaaaccctaggccttttacgaaagaaggaaaagagagctacttcctggccggacctcattgccaaggtttt
ccggatcgatgtgaaggcagatgttgactcgatccaccccactgagttctgccataactgctggagcatcatgcacaggaagttt
agcagtgcaccatgcgaagtgtacttccccagaaacgtgaccatggaatggcaccctcacacacccagctgcgacatctgcaa
cacagccagaagaggcctgaagcggaagtccctgcagcctaatctgcagctgagcaagaaactgaaaaccgtgctggacca
ggccagacaggcccggcaaagaaagagaagggcccaagccagaatcagcagcaaggacgtgatgaagaagatcgcca
actgcagcaagatccacctgagcaccaaactgctggccgtggacttccctgagcacttcgtgaagtccatcagctgccagatctg
cgagcacatcctggccgatcctgtggaaacaaactgcaagcacgtgttctgcagagtgtgcatcctgcggtgcctgaaagtgat
gggcagctactgcccctcctgcagatacccttgcttccccaccgatctggaaagccctgtgaagtccttcctgagcgtgctgaaca
gcctgatggtcaagtgccccgccaaagaatgcaacgaggaagtgtccctggaaaagtacaaccaccacatcagcagccaca
aagagtccaaagaaatcttcgtgcacatcaacaaaggcggcagaccccggcagcatctgctgtctcttacaagacgggccca
gaagcaccggctgagagaactgaagctgcaagtgaaggcctttgccgacaaagaggaaggcggcgacgtcaagagcgtgt
gcatgaccctgtttctgctggccctgagagcccggaatgagcatagacaggccgatgagctggaagccatcatgcaaggcaa
aggcagcggactgcagcctgctgtgtgtctggctatcagagtgaacaccttcctgtcctgcagccagtaccacaagatgtaccgg
accgtgaaggccattaccggcagacagatcttccagcctctgcacgccctgagaaacgccgagaaagttctgctgcctggctac
caccacttcgagtggcagcctccactgaagaacgtgtccagcagcaccgacgtgggcatcatcgatggactgagcggactgtc
tagcagcgtggacgactaccccgtggacacaatcgccaagcggttcagatacgacagcgccctggtgtctgccctgatggaca
tggaagaggacatcctggaaggcatgcggagccaggacctggacgattacctgaacggccctttcaccgtggtggtcaaaga
aagctgtgacggcatgggcgacgtgtccgagaaacacggatctggacctgtggtgccagagaaggccgtgcggttcagcttca
ccatcatgaagatcactatcgcccacagcagccagaacgtgaaagtgttcgaggaagccaagcctaacagcgagctgtgctg
caagcctctgtgtctgatgctggccgacgagagcgatcacgagacactgaccgccattctgagccctctgatcgccgaacggga
agccatgaagtcctccgagctgatgctcgaactcggcggcatcctgagaaccttcaagttcatcttccgcggcaccggctacgac
gagaagctcgttagagaggtggaaggcctggaagcctctggcagcgtgtacatctgcaccctgtgtgacgccaccagactgga
agctagccagaacctggtgttccacagcatcaccagaagccacgccgaaaacctggaaagatacgaagtgtggcggagca
acccctaccacgagagcgtggaagaactgcgggatagagtgaagggcgtgtccgccaagcctttcatcgagacagtgcctag
catcgacgccctgcactgcgatattggcaacgccgccgaattctacaagatctttcagctggaaatcggcgaggtctacaagaa
ccccaacgcctctaaagaggaacggaagcgctggcaggccacactggataagcacctgagaaagaagatgaatctgaagc
ccatcatgaggatgaacggcaacttcgcccggaagctgatgaccaaagaaaccgtggatgccgtgtgcgagctgatcccctct
gaggaaagacacgaggccctgcgggaactgatggacctgtacctgaagatgaagcccgtgtggcggtctagctgtcctgcca
aagagtgccctgagtctctgtgccagtacagcttcaacagccagagattcgccgagctgctgtccaccaagttcaagtacagata
cgagggcaagatcaccaactacttccacaagaccctggctcacgtgcccgagatcatcgagagagatggctctattggcgcct
gggcctctgagggcaatgagtctggcaacaagctgttccggcggttccgcaagatgaacgccagacagagcaagtgctacga
gatggaagatgtgctgaagcaccactggctgtacaccagcaagtacctgcagaaattcatgaacgcccacaacgccctcaag
accagcggctttaccatgaatcctcaggccagcctgggcgatcctttaggcatagaggactctctggaaagccaagattcaatgg
aattttaagtagggcaaccacttatgagttggtttttgcaattgagtttccctctgggttgcattgagggcttctcctagcaccctttactg
ctgtgtatggggcttcaccatccaagaggtggtaggttggagtaagatgctacagatgctctcaagtcaggaatagaaactgatg
agctgattgcttgaggcttttagtgagttccgaaaagcaacaggaaaaatcagttatctgaaagctcagtaactcagaacaggag
taactgcaggggaccagagatgagcaaagatctgtgtgtgttggggagctgtcatgtaaatcaaagccaaggttgtcaaagaac
agccagtgaggccaggaaagaaattggtcttgtggttttcatttttttcccccttgattgattatattttgtattgagatatgataagtgcctt
ctatttcatttttgaataattcttcatttttataattttacatatcttggcttgctatataagattcaaaagagctttttaaatttttctaataatatct
tacatttgtacagcatgatgacctttacaaagtgctctcaatgcatttacccattcgttatataaatatgttacatcaggacaactttgag
aaaatcagtccttttttatgtttaaattatgtatctattgtaaccttcagagtttaggaggtcatctgctgtcatggatttttcaataatgaatt
tagaatacacctgttagctacagttagttattaaatcttctgataatatatgtttacttagctatcagaagccaagtatgattctttattttta
ctttttcatttcaagaaatttagagtttccaaatttagagcttctgcatacagtcttaaagccacagaggcttgtaaaaatataggttag
cttgatgtctaaaaatatatttcatgtcttactgaaacattttgccagactttctccaaatgaaacctgaatcaatttttctaaatctaggttt
catagagtcctctcctctgcaatgtgttattctttctataatgatcagtttactttcagtggattcagaattgtgtagcaggataaccttgtat
ttttccatccgctaagtttagatggagtccaaacgcagtacagcagaagagtt
Left HA
ttcagcacccacatattaaattttcagaatggaaatttaagctgttccgggtgagatcctttgaaaagacacctgaagaagctcaa
aaggaaaagaaggattcctttgaggggaaaccctctctggagcaatctccagcagtcctggacaaggctgatggtcagaagcc
agtcccaactcagccattgttaaaagcccaccctaagttttcaaagaaatttcacgacaacgagaaagcaagaggcaaagcg
atccatcaagccaaccttcgacatctctgccgcatctgtgggaattcttttagagctgatgagcacaacaggagatatccagtccat
ggtcctgtggatggtaaaaccctaggccttttacgaaagaaggaaaagagagctacttcctggccggacctcattgccaaggtttt
ccggatcgatgtgaaggcagatgttgactcgatccaccccactgagttctgccataactgctggagcatcatgcacaggaagttt
agcagtgcaccat
coRAG1 CDS
gcgaagtgtacttccccagaaacgtgaccatggaatggcaccctcacacacccagctgcgacatctgcaacacagccagaag
aggcctgaagcggaagtccctgcagcctaatctgcagctgagcaagaaactgaaaaccgtgctggaccaggccagacaggc
ccggcaaagaaagagaagggcccaagccagaatcagcagcaaggacgtgatgaagaagatcgccaactgcagcaagat
ccacctgagcaccaaactgctggccgtggacttccctgagcacttcgtgaagtccatcagctgccagatctgcgagcacatcctg
gccgatcctgtggaaacaaactgcaagcacgtgttctgcagagtgtgcatcctgcggtgcctgaaagtgatgggcagctactgc
ccctcctgcagatacccttgcttccccaccgatctggaaagccctgtgaagtccttcctgagcgtgctgaacagcctgatggtcaa
gtgccccgccaaagaatgcaacgaggaagtgtccctggaaaagtacaaccaccacatcagcagccacaaagagtccaaag
aaatcttcgtgcacatcaacaaaggcggcagaccccggcagcatctgctgtctcttacaagacgggcccagaagcaccggctg
agagaactgaagctgcaagtgaaggcctttgccgacaaagaggaaggcggcgacgtcaagagcgtgtgcatgaccctgtttct
gctggccctgagagcccggaatgagcatagacaggccgatgagctggaagccatcatgcaaggcaaaggcagcggactgc
agcctgctgtgtgtctggctatcagagtgaacaccttcctgtcctgcagccagtaccacaagatgtaccggaccgtgaaggccatt
accggcagacagatcttccagcctctgcacgccctgagaaacgccgagaaagttctgctgcctggctaccaccacttcgagtgg
cagcctccactgaagaacgtgtccagcagcaccgacgtgggcatcatcgatggactgagcggactgtctagcagcgtggacg
actaccccgtggacacaatcgccaagcggttcagatacgacagcgccctggtgtctgccctgatggacatggaagaggacatc
ctggaaggcatgcggagccaggacctggacgattacctgaacggccctttcaccgtggtggtcaaagaaagctgtgacggcat
gggcgacgtgtccgagaaacacggatctggacctgtggtgccagagaaggccgtgcggttcagcttcaccatcatgaagatca
ctatcgcccacagcagccagaacgtgaaagtgttcgaggaagccaagcctaacagcgagctgtgctgcaagcctctgtgtctg
atgctggccgacgagagcgatcacgagacactgaccgccattctgagccctctgatcgccgaacgggaagccatgaagtcctc
cgagctgatgctcgaactcggcggcatcctgagaaccttcaagttcatcttccgcggcaccggctacgacgagaagctcgttag
agaggtggaaggcctggaagcctctggcagcgtgtacatctgcaccctgtgtgacgccaccagactggaagctagccagaac
ctggtgttccacagcatcaccagaagccacgccgaaaacctggaaagatacgaagtgtggcggagcaacccctaccacgag
agcgtggaagaactgcgggatagagtgaagggcgtgtccgccaagcctttcatcgagacagtgcctagcatcgacgccctgc
actgcgatattggcaacgccgccgaattctacaagatctttcagctggaaatcggcgaggtctacaagaaccccaacgcctcta
aagaggaacggaagcgctggcaggccacactggataagcacctgagaaagaagatgaatctgaagcccatcatgaggatg
aacggcaacttcgcccggaagctgatgaccaaagaaaccgtggatgccgtgtgcgagctgatcccctctgaggaaagacacg
aggccctgcgggaactgatggacctgtacctgaagatgaagcccgtgtggcggtctagctgtcctgccaaagagtgccctgagt
ctctgtgccagtacagcttcaacagccagagattcgccgagctgctgtccaccaagttcaagtacagatacgagggcaagatca
ccaactacttccacaagaccctggctcacgtgcccgagatcatcgagagagatggctctattggcgcctgggcctctgagggca
atgagtctggcaacaagctgttccggcggttccgcaagatgaacgccagacagagcaagtgctacgagatggaagatgtgctg
aagcaccactggctgtacaccagcaagtacctgcagaaattcatgaacgcccacaacgccctcaagaccagcggctttaccat
gaatcctcaggccagcctgggcgatccttt
Right HA
aggcatagaggactctctggaaagccaagattcaatggaattttaagtagggcaaccacttatgagttggtttttgcaattgagtttc
cctctgggttgcattgagggcttctcctagcaccctttactgctgtgtatggggcttcaccatccaagaggtggtaggttggagtaag
atgctacagatgctctcaagtcaggaatagaaactgatgagctgattgcttgaggcttttagtgagttccgaaaagcaacaggaa
aaatcagttatctgaaagctcagtaactcagaacaggagtaactgcaggggaccagagatgagcaaagatctgtgtgtgttggg
gagctgtcatgtaaatcaaagccaaggttgtcaaagaacagccagtgaggccaggaaagaaattggtcttgtggttttcatttttttc
ccccttgattgattatattttgtattgagatatgataagtgccttctatttcatttttgaataattcttcatttttataattttacatatcttgg
cttgctatataagattcaaaagagctttttaaatttttctaataatatcttacatttgtacagcatgatgacctttacaaagtgctctcaatgca
tttacccattcgttatataaatatgttacatcaggacaactttgagaaaatcagtccttttttatgtttaaattatgtatctattgtaaccttcag
agtttaggaggtcatctgctgtcatggatttttcaataatgaatttagaatacacctgttagctacagttagttattaaatcttctgataat
atatgtttacttagctatcagaagccaagtatgattctttatttttactttttcatttcaagaaatttagagtttccaaatttagagcttctgcat
acagtcttaaagccacagaggcttgtaaaaatataggttagcttgatgtctaaaaatatatttcatgtcttactgaaacattttgccag
actttctccaaatgaaacctgaatcaatttttctaaatctaggtttcatagagtcctctcctctgcaatgtgttattctttctataatgatcag
tttactttcagtggattcagaattgtgtagcaggataaccttgtatttttccatccgctaagtttagatggagtccaaacgcagtacagc
agaagagtt
DONOR with short R-HA specific for “g6 exon2 M2” gRNA for the exon 2 RAG1 gene
replacement strategy
INSERT
gagcacaacaggagatatccagtccatggtcctgtggatggtaaaaccctaggccttttacgaaagaaggaaaagagagcta
cttcctggccggacctcattgccaaggttttccggatcgatgtgaaggcagatgttgactcgatccaccccactgagttctgccata
actgctggagcatcatgcacaggaagtttagcagtgccccatgtgaggtttacttcccgaggaatgtcactatggaatggcaccct
cacacacccagctgcgacatctgcaacacagccagaagaggcctgaagcggaagtccctgcagcctaatctgcagctgagc
aagaaactgaaaaccgtgctggaccaggccagacaggcccggcaaagaaagagaagggcccaagccagaatcagcag
caaggacgtgatgaagaagatcgccaactgcagcaagatccacctgagcaccaaactgctggccgtggacttccctgagcac
ttcgtgaagtccatcagctgccagatctgcgagcacatcctggccgatcctgtggaaacaaactgcaagcacgtgttctgcagag
tgtgcatcctgcggtgcctgaaagtgatgggcagctactgcccctcctgcagatacccttgcttccccaccgatctggaaagccct
gtgaagtccttcctgagcgtgctgaacagcctgatggtcaagtgccccgccaaagaatgcaacgaggaagtgtccctggaaaa
gtacaaccaccacatcagcagccacaaagagtccaaagaaatcttcgtgcacatcaacaaaggggcagaccccggcagc
atctgctgtctcttacaagacgggcccagaagcaccggctgagagaactgaagctgcaagtgaaggcctttgccgacaaaga
ggaaggcggcgacgtcaagagcgtgtgcatgaccctgtttctgctggccctgagagcccggaatgagcatagacaggccgat
gagctggaagccatcatgcaaggcaaaggcagcggactgcagcctgctgtgtgtctggctatcagagtgaacaccttcctgtcct
gcagccagtaccacaagatgtaccggaccgtgaaggccattaccggcagacagatcttccagcctctgcacgccctgagaaa
cgccgagaaagttctgctgcctggctaccaccacttcgagtggcagcctccactgaagaacgtgtccagcagcaccgacgtgg
gcatcatcgatggactgagcggactgtctagcagcgtggacgactaccccgtggacacaatcgccaagcggttcagatacgac
agcgccctggtgtctgccctgatggacatggaagaggacatcctggaaggcatgcggagccaggacctggacgattacctga
acggccctttcaccgtggtggtcaaagaaagctgtgacggcatgggcgacgtgtccgagaaacacggatctggacctgtggtg
ccagagaaggccgtgcggttcagcttcaccatcatgaagatcactatcgcccacagcagccagaacgtgaaagtgttcgagga
agccaagcctaacagcgagctgtgctgcaagcctctgtgtctgatgctggccgacgagagcgatcacgagacactgaccgcc
attctgagccctctgatcgccgaacgggaagccatgaagtcctccgagctgatgctcgaactcggcggcatcctgagaaccttca
agttcatcttccgcggcaccggctacgacgagaagctcgttagagaggtggaaggcctggaagcctctggcagcgtgtacatct
gcaccctgtgtgacgccaccagactggaagctagccagaacctggtgttccacagcatcaccagaagccacgccgaaaacct
ggaaagatacgaagtgtggcggagcaacccctaccacgagagcgtggaagaactgcgggatagagtgaagggcgtgtccg
ccaagcctttcatcgagacagtgcctagcatcgacgccctgcactgcgatattggcaacgccgccgaattctacaagatctttcag
ctggaaatcggcgaggtgtacaagaaccccaacgcctctaaagaggaacggaagcgctggcaggccacactggataagca
cctgagaaagaagatgaatctgaagcccatcatgaggatgaacggcaacttcgcccggaagctgatgaccaaagaaaccgt
ggatgccgtgtgcgagctgatcccctctgaggaaagacacgaggccctgcgggaactgatggacctgtacctgaagatgaag
cccgtgtggcggtctagctgtcctgccaaagagtgccctgagtctctgtgccagtacagcttcaacagccagagattcgccgagct
gctgtccaccaagttcaagtacagatacgagggcaagatcaccaactacttccacaagaccctggctcacgtgcccgagatcat
cgagagagatggctctattggcgcctgggcctctgagggcaatgagtctggcaacaagctgttccggcggttccgcaagatgaa
cgccagacagagcaagtgctacgagatggaagatgtgctgaagcaccactggctgtacaccagcaagtacctgcagaaattc
atgaacgcccacaacgccctcaagaccagcggctttaccatgaatcctcaggccagcctgggcgatcctttaggcatagagga
ctctctggaaagccaagattcaatggaattttaagtagggcaaccacttatgagttggtttttgcaattgagtttccctctgggttgcatt
gagggcttctcctagcaccctttactgctgtgtatggggcttcaccatccaagaggtggtaggttggagtaagatgctacagatgct
ctcaagtcaggaatagaaactgatgagctgattgcttgaggcttttagtgagttccgaaaagcaacaggaaaaatcagttatctga
aagctcagtaactcagaacaggagtaactgcaggggaccagagatgagcaaagatctgtgtgtgttggggagctgtcatgtaa
atcaaagccaaggttgtcaaagaacagccagtgaggccaggaaagaaattggtcttgtggttttcatttttttcccccttgattgatta
tattttgtattgagatatgataagtgccttctatttcatttttgaataattcttcatttttataattttacatatcttggcttgctatataagattcaa
aagagctttttaaatttttctaataatatcttacatttgtacagcatgatgacctttacaaagtgctctcaatgcatttacccattcgttatat
aaatatgttacatcaggacaactttgagaaaatcagtccttttttatgtttaaattatgtatctattgtaaccttcagagtttaggaggtca
tctgctgtcatggatttttcaataatgaatttagaatacacctgttagctacagttagttattaaatcttctgataatatatgtttacttagcta
tcagaagccaagtatgattctttatttttactttttcatttcaagaaatttagagtttccaaatttagagct
Left_HA
gagcacaacaggagatatccagtccatggtcctgtggatggtaaaaccctaggccttttacgaaagaaggaaaagagagcta
cttcctggccggacctcattgccaaggttttccggatcgatgtgaaggcagatgttgactcgatccaccccactgagttctgccata
actgctggagcatcatgcacaggaagtttagcagtgccccatgtgaggtttacttcccgaggaatgtcactatg
coRAG1 CDS
gaatggcaccctcacacacccagctgcgacatctgcaacacagccagaagaggcctgaagcggaagtccctgcagcctaat
ctgcagctgagcaagaaactgaaaaccgtgctggaccaggccagacaggcccggcaaagaaagagaagggcccaagcc
agaatcagcagcaaggacgtgatgaagaagatcgccaactgcagcaagatccacctgagcaccaaactgctggccgtgga
cttccctgagcacttcgtgaagtccatcagctgccagatctgcgagcacatcctggccgatcctgtggaaacaaactgcaagcac
gtgttctgcagagtgtgcatcctgcggtgcctgaaagtgatgggcagctactgcccctcctgcagatacccttgcttccccaccgat
ctggaaagccctgtgaagtccttcctgagcgtgctgaacagcctgatggtcaagtgccccgccaaagaatgcaacgaggaagt
gtccctggaaaagtacaaccaccacatcagcagccacaaagagtccaaagaaatcttcgtgcacatcaacaaaggcggcag
accccggcagcatctgctgtctcttacaagacgggcccagaagcaccggctgagagaactgaagctgcaagtgaaggcctttg
ccgacaaagaggaaggcggcgacgtcaagagcgtgtgcatgaccctgtttctgctggccctgagagcccggaatgagcatag
acaggccgatgagctggaagccatcatgcaaggcaaaggcagcggactgcagcctgctgtgtgtctggctatcagagtgaac
accttcctgtcctgcagccagtaccacaagatgtaccggaccgtgaaggccattaccggcagacagatcttccagcctctgcac
gccctgagaaacgccgagaaagttctgctgcctggctaccaccacttcgagtggcagcctccactgaagaacgtgtccagcag
caccgacgtgggcatcatcgatggactgagcggactgtctagcagcgtggacgactaccccgtggacacaatcgccaagcgg
ttcagatacgacagcgccctggtgtctgccctgatggacatggaagaggacatcctggaaggcatgcggagccaggacctgg
acgattacctgaacggccctttcaccgtggtggtcaaagaaagctgtgacggcatgggcgacgtgtccgagaaacacggatct
ggacctgtggtgccagagaaggccgtgcggttcagcttcaccatcatgaagatcactatcgcccacagcagccagaacgtgaa
agtgttcgaggaagccaagcctaacagcgagctgtgctgcaagcctctgtgtctgatgctggccgacgagagcgatcacgaga
cactgaccgccattctgagccctctgatcgccgaacgggaagccatgaagtcctccgagctgatgctcgaactcggcggcatcc
tgagaaccttcaagttcatcttccgcggcaccggctacgacgagaagctcgttagagaggtggaaggcctggaagcctctggc
agcgtgtacatctgcaccctgtgtgacgccaccagactggaagctagccagaacctggtgttccacagcatcaccagaagcca
cgccgaaaacctggaaagatacgaagtgtggcggagcaacccctaccacgagagcgtggaagaactgcgggatagagtga
agggcgtgtccgccaagcctttcatcgagacagtgcctagcatcgacgccctgcactgcgatattggcaacgccgccgaattcta
caagatctttcagctggaaatcggcgaggtgtacaagaaccccaacgcctctaaagaggaacggaagcgctggcaggccac
actggataagcacctgagaaagaagatgaatctgaagcccatcatgaggatgaacggcaacttcgcccggaagctgatgacc
aaagaaaccgtggatgccgtgtgcgagctgatcccctctgaggaaagacacgaggccctgcgggaactgatggacctgtacc
tgaagatgaagcccgtgtggcggtctagctgtcctgccaaagagtgccctgagtctctgtgccagtacagcttcaacagccagag
attcgccgagctgctgtccaccaagttcaagtacagatacgagggcaagatcaccaactacttccacaagaccctggctcacgt
gcccgagatcatcgagagagatggctctattggcgcctgggcctctgagggcaatgagtctggcaacaagctgttccggcggtt
ccgcaagatgaacgccagacagagcaagtgctacgagatggaagatgtgctgaagcaccactggctgtacaccagcaagta
cctgcagaaattcatgaacgcccacaacgccctcaagaccagcggctttaccatgaatcctcaggccagcctgggcgatccttt
Right HA
aggcatagaggactctctggaaagccaagattcaatggaattttaagtagggcaaccacttatgagttggtttttgcaattgagtttc
cctctgggttgcattgagggcttctcctagcaccctttactgctgtgtatggggcttcaccatccaagaggtggtaggttggagtaag
atgctacagatgctctcaagtcaggaatagaaactgatgagctgattgcttgaggcttttagtgagttccgaaaagcaacaggaa
aaatcagttatctgaaagctcagtaactcagaacaggagtaactgcaggggaccagagatgagcaaagatctgtgtgtgttggg
gagctgtcatgtaaatcaaagccaaggttgtcaaagaacagccagtgaggccaggaaagaaattggtcttgtggttttcatttttttc
ccccttgattgattatattttgtattgagatatgataagtgccttctatttcatttttgaataattcttcatttttataattttacatatcttggcttgc
tatataagattcaaaagagctttttaaatttttctaataatatcttacatttgtacagcatgatgacctttacaaagtgctctcaatgcattt
acccattcgttatataaatatgttacatcaggacaactttgagaaaatcagtccttttttatgtttaaattatgtatctattgtaaccttcag
agtttaggaggtcatctgctgtcatggatttttcaataatgaatttagaatacacctgttagctacagttagttattaaatcttctgataat
atatgtttacttagctatcagaagccaagtatgattctttatttttactttttcatttcaagaaatttagagtttccaaatttagagct

### Production of Additional AAV6 Donors

AAV6 donor production was performed by the vector core facility at the Telethon Institute of Genetics and Medicine (TIGEM), Pozzuoli (NA, Italy). Briefly, AAV vectors were produced by transient triple transfection of HEK293 cells by calcium phosphate. The following day, the medium was changed with serum-free DMEM and cells were harvested 72 hours after transfection. Cells were lysed by three rounds of freeze-thaw to release the viral particles and the lysate was incubated with DNAseI and RNAseI to eliminate nucleic acids. AAV vector was then purified by two sequential rounds of Cesium Chloride (CsCl2) gradient. For each viral preparation, physical titers (genome copies/mL) were determined by PCR quantification using TaqMan.

### Statistical Analysis

When normality assumptions were not met, non-parametric statistical tests were performed. Mann-Whitney test was used for non-paired comparisons, while Wilcoxon matched-pairs test was used for paired comparisons. Values are expressed as Mean±SD and P values are showed as: *<0.05; **<0.005; ***<0.0005; ****<0.0001.

All publications mentioned in the above specification are herein incorporated by reference. Various modifications and variations of the disclosed polynucleotides, vectors, RNAs, methods, cells, kits, compositions, systems and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

### EMBODIMENTS

Various features and embodiments of the present invention will now be described with reference to the following numbered paragraphs (paras).

1. An isolated polynucleotide comprising from 5′ to 3′: a first homology region, a nucleotide sequence encoding a RAG1 polypeptide fragment, and a second homology region, wherein the first homology region is homologous to a first region of the RAG1 exon 2 and the second homology region is homologous to a second region of the RAG1 exon 2.

2. The isolated polynucleotide according to para 1, wherein:
(i) the first homology region is homologous to a region upstream of chr 11:36574368 and the second homology region is homologous to a region downstream of chr 11:36574369;
(ii) the first homology region is homologous to a region upstream of chr 11:36574367 and the second homology region is homologous to a region downstream of chr 11:36574368;
(iii) the first homology region is homologous to a region upstream of chr 11:36574394 and the second homology region is homologous to a region downstream of chr 11:36574395;
(iv) the first homology region is homologous to a region upstream of chr 11:36574294 and the second homology region is homologous to a region downstream of chr 11:36574295;
(v) the first homology region is homologous to a region upstream of chr 11:36574109 and the second homology region is homologous to a region downstream of chr 11:36574110;
(vi) the first homology region is homologous to a region upstream of chr 11:36573910 and the second homology region is homologous to a region downstream of chr 11:36573911;
(vii) the first homology region is homologous to a region upstream of chr 11:36573878 and the second homology region is homologous to a region downstream of chr 11:36573879;
(viii) the first homology region is homologous to a region upstream of chr 11:36573959 and the second homology region is homologous to a region downstream of chr 11:36573960;
(ix) the first homology region is homologous to a region upstream of chr 11:36573957 and the second homology region is homologous to a region downstream of chr 11:36573958;
(x) the first homology region is homologous to a region upstream of chr 11:36573879 and the second homology region is homologous to a region downstream of chr 11:36573880;
(xi) the first homology region is homologous to a region upstream of chr 11:36573892 and the second homology region is homologous to a region downstream of chr 11:36573893;
(xii) the first homology region is homologous to a region upstream of chr 11:36573955 and the second homology region is homologous to a region downstream of chr 11:36573956;
(xiii) the first homology region is homologous to a region upstream of chr 11:36573878 and the second homology region is homologous to a region downstream of chr 11:36573879; or
(xiv) the first homology region is homologous to a region upstream of chr 11:36574406 and the second homology region is homologous to a region downstream of chr 11:36574407.

3. The isolated polynucleotide according to para 1 or 2, wherein:
(v) the first homology region is homologous to a region upstream of chr 11:36574109 and the second homology region is homologous to a region downstream of chr 11:36574110; or
(vi) the first homology region is homologous to a region upstream of chr 11:36573910 and the second homology region is homologous to a region downstream of chr 11:36573911.

4. The isolated polynucleotide according to any preceding para, wherein:
(i) the first homology region is homologous to a region comprising chr 11:36574319-36574368 and/or the second homology region is homologous to a region comprising chr 11:36574369-36574418;
(ii) the first homology region is homologous to a region comprising chr 11:36574318-36574367 and/or the second homology region is homologous to a region comprising chr 11:36574368-36574417;
(iii) the first homology region is homologous to a region comprising chr 11:36574345-36574394 and/or the second homology region is homologous to a region comprising chr 11:36574395-36574444;
(iv) the first homology region is homologous to a region comprising chr 11:36574245-36574294 and/or the second homology region is homologous to a region comprising chr 11:36574295-36574344;
(v) the first homology region is homologous to a region comprising chr 11:36574060-36574109 and/or the second homology region is homologous to a region comprising chr 11:36574110-36574159;
(vi) the first homology region is homologous to a region comprising chr 11:36573861-36573910 and/or the second homology region is homologous to a region comprising chr 11:36573911-36573960;
(vii) the first homology region is homologous to a region comprising chr 11:36573829-36573878 and/or the second homology region is homologous to a region comprising chr 11:36573879-36573928;
(viii) the first homology region is homologous to a region comprising chr 11:36573910-36573959 and/or the second homology region is homologous to a region comprising chr 11:36573960-36574009;
(ix) the first homology region is homologous to a region comprising chr 11:36573908-36573957 and/or the second homology region is homologous to a region comprising chr 11:36573958-36574007;
(x) the first homology region is homologous to a region comprising chr 11:36573830-36573879 and/or the second homology region is homologous to a region comprising chr 11:36573880-36573929;
(xi) the first homology region is homologous to a region comprising chr 11:36573843-36573892 and/or the second homology region is homologous to a region comprising chr 11:36573893-36573942;
(xii) the first homology region is homologous to a region comprising chr 11:36573906-36573955 and/or the second homology region is homologous to a region comprising chr 11:36573956-36574005;
(xiii) the first homology region is homologous to a region comprising chr 11:36573829-36573878 and/or the second homology region is homologous to a region comprising chr 11:36573879-36573928; or
(xiv) the first homology region is homologous to a region comprising chr 11:36574357-36574406 and/or the second homology region is homologous to a region comprising chr 11:36574407-36574456.

5. The isolated polynucleotide according to any preceding para, wherein:
(i) the 3′ terminal sequence of the first homology region comprises or consists of a nucleotide sequence that has at least 70% identity to SEQ ID NO: 25 and/or the 5′ terminal sequence of the second homology region comprises or consists of a nucleotide sequence that has at least 70% identity to SEQ ID NO: 45;
(ii) the 3′ terminal sequence of the first homology region comprises or consists of a nucleotide sequence that has at least 70% identity to SEQ ID NO: 26 and/or the 5′ terminal sequence of the second homology region comprises or consists of a nucleotide sequence that has at least 70% identity to SEQ ID NO: 46;
(iii) the 3′ terminal sequence of the first homology region comprises or consists of a nucleotide sequence that has at least 70% identity to SEQ ID NO: 27 and/or the 5′ terminal sequence of the second homology region comprises or consists of a nucleotide sequence that has at least 70% identity to SEQ ID NO: 47;
(iv) the 3′ terminal sequence of the first homology region comprises or consists of a nucleotide sequence that has at least 70% identity to SEQ ID NO: 28 and/or the 5′ terminal sequence of the second homology region comprises or consists of a nucleotide sequence that has at least 70% identity to SEQ ID NO: 48;
(v) the 3′ terminal sequence of the first homology region comprises or consists of a nucleotide sequence that has at least 70% identity to SEQ ID NO: 29 or SEQ ID NO: 39 and/or the 5′ terminal sequence of the second homology region comprises or consists of a nucleotide sequence that has at least 70% identity to SEQ ID NO: 49 or SEQ ID NO: 59;
(vi) the 3′ terminal sequence of the first homology region comprises or consists of a nucleotide sequence that has at least 70% identity to SEQ ID NO: 30 or SEQ ID NO: 40 and/or the 5′ terminal sequence of the second homology region comprises or consists of a nucleotide sequence that has at least 70% identity to SEQ ID NO: 50 or SEQ ID NO: 60;
(vii) the 3′ terminal sequence of the first homology region comprises or consists of a nucleotide sequence that has at least 70% identity to SEQ ID NO: 31 or SEQ ID NO: 41 and/or the 5′ terminal sequence of the second homology region comprises or consists of a nucleotide sequence that has at least 70% identity to SEQ ID NO: 51;
(viii) the 3′ terminal sequence of the first homology region comprises or consists of a nucleotide sequence that has at least 70% identity to SEQ ID NO: 32 or SEQ ID NO: 42 and/or the 5′ terminal sequence of the second homology region comprises or consists of a nucleotide sequence that has at least 70% identity to SEQ ID NO: 52;
(ix) the 3′ terminal sequence of the first homology region comprises or consists of a nucleotide sequence that has at least 70% identity to SEQ ID NO: 33 or SEQ ID NO: 42 and/or the 5′ terminal sequence of the second homology region comprises or consists of a nucleotide sequence that has at least 70% identity to SEQ ID NO: 53;
(x) the 3′ terminal sequence of the first homology region comprises or consists of a nucleotide sequence that has at least 70% identity to SEQ ID NO: 34 or SEQ ID NO: 41 and/or the 5′ terminal sequence of the second homology region comprises or consists of a nucleotide sequence that has at least 70% identity to SEQ ID NO: 54;
(xi) the 3′ terminal sequence of the first homology region comprises or consists of a nucleotide sequence that has at least 70% identity to SEQ ID NO: 35 or SEQ ID NO: 41 and/or the 5′ terminal sequence of the second homology region comprises or consists of a nucleotide sequence that has at least 70% identity to SEQ ID NO: 55;
(xii) the 3′ terminal sequence of the first homology region comprises or consists of a nucleotide sequence that has at least 70% identity to SEQ ID NO: 36 or SEQ ID NO: 42 and/or the 5′ terminal sequence of the second homology region comprises or consists of a nucleotide sequence that has at least 70% identity to SEQ ID NO: 56;
(xiii) the 3′ terminal sequence of the first homology region comprises or consists of a nucleotide sequence that has at least 70% identity to SEQ ID NO: 37 or SEQ ID NO: 43 and/or the 5′ terminal sequence of the second homology region comprises or consists of a nucleotide sequence that has at least 70% identity to SEQ ID NO: 57; or
(xiv) the 3′ terminal sequence of the first homology region comprises or consists of a nucleotide sequence that has at least 70% identity to SEQ ID NO: 38 or SEQ ID NO: 44 and/or the 5′ terminal sequence of the second homology region comprises or consists of a nucleotide sequence that has at least 70% identity to SEQ ID NO: 58.

6. The isolated polynucleotide according to any preceding para, wherein:
(1) the first homology region comprises or consists of a nucleotide sequence that has at least 70% identity to SEQ ID NO: 69, or a fragment thereof and/or the second homology region comprises or consists of a nucleotide sequence that has at least 70% identity to SEQ ID NO: 77, or a fragment thereof; or
(4) the first homology region comprises or consists of a nucleotide sequence that has at least 70% identity to SEQ ID NO: 71, or a fragment thereof and/or the second homology region comprises or consists of a nucleotide sequence that has at least 70% identity to SEQ ID NO: 78, or a fragment thereof.

7. The isolated polynucleotide according to any preceding para, wherein the first and second homology regions are each 50-2000 bp in length, 50-1800 bp in length, 50-1500 bp in length, 50-1000 bp in length, 100-500 bp in length, or 200-400 bp in length.

8 An isolated polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a first region of the RAG1 intron 1 or exon 2 and the second homology region is homologous to a second region of the RAG1 exon 2.

9. The isolated polynucleotide according to para 8, wherein the first homology region is homologous to a region upstream of: (i) chr 11:36569295; (ii) chr 11:36573790; (iii) chr 11:36573641; (iv) chr 11:36573351; (v) chr 11:36569080; (vi) chr 11:36572472; (vii) chr 11:36571458; (viii) chr 11:36571366; (ix) chr 11:36572859 (x) chr 11:36571457; (xi) chr 11:36569351; or (xii) chr 11:36572375, preferably wherein the first homology region is homologous to a region upstream of: (i) chr 11:36569295; (ii) chr 11:36573351; (iii) chr 11:36571366, more preferably wherein the first homology region is homologous to a region upstream of chr 11:36569295.

10. The isolated polynucleotide according to para 8 or 9, wherein the first homology region is homologous to a region comprising chr 11:36569245-chr 11:36569294, preferably wherein the 3′ terminal sequence of the first homology region comprises or consists of a nucleotide sequence that has at least 70% identity to SEQ ID NO: 81, more preferably wherein the first homology region comprises or consists of a nucleotide sequence that has at least 70% identity to SEQ ID NO: 93.

11. The isolated polynucleotide according to any preceding para, wherein the second homology region is homologous to a region downstream of chr 11:36574557; downstream of chr 11:36574870; downstream of chr 11:36575183; downstream of chr 11:36575496; downstream of chr 11:36575810; downstream of chr 11:36576123; or downstream of chr 11:36576436, preferably wherein the second homology region is homologous to a region comprising chr 11:36576437-chr 11:36576536.

12. The isolated polynucleotide according to any preceding para, wherein the second homology region comprises or consists of a nucleotide sequence that has at least 70% identity to any of SEQ ID NOs: 79-80 or 94, or a fragment thereof, preferably wherein the 5′ terminal sequence of the second homology region comprises or consists of a nucleotide sequence that has at least 70% identity to SEQ ID NO: 67.

13. The isolated polynucleotide according to any of paras 1 to 7 or paras 11 or 12, wherein:
(2) the first homology region comprises or consists of a nucleotide sequence that has at least 70% identity to SEQ ID NO: 70, or a fragment thereof and/or the second homology region comprises or consists of a nucleotide sequence that has at least 70% identity to SEQ ID NO: 79, or a fragment thereof;
(3) the first homology region comprises or consists of a nucleotide sequence that has at least 70% identity to SEQ ID NO: 70, or a fragment thereof and/or the second homology region comprises or consists of a nucleotide sequence that has at least 70% identity to SEQ ID NO: 80, or a fragment thereof;
(5) the first homology region comprises or consists of a nucleotide sequence that has at least 70% identity to SEQ ID NO: 72, or a fragment thereof and/or the second homology region comprises or consists of a nucleotide sequence that has at least 70% identity to SEQ ID NO: 79, or a fragment thereof;
(6) the first homology region comprises or consists of a nucleotide sequence that has at least 70% identity to SEQ ID NO: 72, or a fragment thereof and/or the second homology region comprises or consists of a nucleotide sequence that has at least 70% identity to SEQ ID NO: 80, or a fragment thereof;
(7) the first homology region comprises or consists of a nucleotide sequence that has at least 70% identity to SEQ ID NO: 73, or a fragment thereof and/or the second homology region comprises or consists of a nucleotide sequence that has at least 70% identity to SEQ ID NO: 79, or a fragment thereof;
(8) the first homology region comprises or consists of a nucleotide sequence that has at least 70% identity to SEQ ID NO: 74, or a fragment thereof and/or the second homology region comprises or consists of a nucleotide sequence that has at least 70% identity to SEQ ID NO: 79, or a fragment thereof;
(9) the first homology region comprises or consists of a nucleotide sequence that has at least 70% identity to SEQ ID NO: 75, or a fragment thereof and/or the second homology region comprises or consists of a nucleotide sequence that has at least 70% identity to SEQ ID NO: 79, or a fragment thereof; or
(10) the first homology region comprises or consists of a nucleotide sequence that has at least 70% identity to SEQ ID NO: 76, or a fragment thereof and/or the second homology region comprises or consists of a nucleotide sequence that has at least 70% identity to SEQ ID NO: 79, or a fragment thereof.

14. The isolated polynucleotide according to any of paras 8 to 12, wherein:
(11) the first homology region comprises or consists of a nucleotide sequence that has at least 70% identity to SEQ ID NO: 93, or a fragment thereof and/or the second homology region comprises or consists of a nucleotide sequence that has at least 70% identity to SEQ ID NO: 94, or a fragment thereof.

15. The isolated polynucleotide according to any preceding para, wherein the first homology region is about 50-1000 bp in length, 100-500 bp in length, or 200-400 bp in length; and/or wherein the second homology region is about 500-2000 bp in length, 1000-2000 bp in length, or 1500-2000 bp in length.

16. The isolated polynucleotide according to any preceding para, wherein the nucleotide sequence encoding a RAG1 polypeptide fragment comprises or consists of a nucleotide sequence encoding a fragment of an amino acid sequence that has at least 70% identity to SEQ ID NO: 4, SEQ ID NO: 5, or SEQ ID NO: 6.

17. The isolated polynucleotide according to any preceding para, wherein the RAG1 polypeptide fragment is at least 500 amino acids in length, at least 550 amino acids in length, at least 600 amino acids in length, at least 650 amino acids in length, at least 700 amino acids in length, at least 750 amino acids in length, or at least 800 amino acids in length.

18. The isolated polynucleotide according to any preceding para, wherein the RAG1 polypeptide fragment comprises or consists of an amino acid sequence that has at least 70% identity to any one of SEQ ID NOs: 7 to 14.

19. The isolated polynucleotide according to any preceding para, wherein the nucleotide sequence encoding a RAG1 polypeptide fragment comprises or consists of a fragment of a nucleotide sequence that has at least 70% identity to SEQ ID NO: 15.

20. The isolated polynucleotide according to any preceding para, wherein the nucleotide sequence encoding a RAG1 polypeptide fragment comprises or consists of a nucleotide sequence that has at least 70% identity to any one of SEQ ID NOs: 17 to 24.

21. The isolated polynucleotide according to any of paras 8 to 12 or paras 14 to 20, wherein the splice acceptor site comprises or consists of a nucleotide sequence that has at least 70% identity to SEQ ID NO: 95.

22. The isolated polynucleotide according to para 1, wherein the polynucleotide comprises or consists of a nucleotide sequence that has at least 70% identity to any one of SEQ ID NOs: 106 to 115.

23. The isolated polynucleotide according to para 8, wherein the polynucleotide comprises or consists of a nucleotide sequence that has at least 70% identity to SEQ ID NO: 116.

24. A vector comprising the polynucleotide according to any preceding para.

25. The vector according to para 24, wherein the vector is a viral vector, optionally an adeno-associated viral (AAV) vector such as an AAV6 vector.

26. A guide RNA comprising or consisting of a nucleotide sequence that has at least 90% identity to any of SEQ ID NOs: 117-130, optionally wherein the guide RNA comprises or consists of a nucleotide sequence that has at least 90% identity to SEQ ID NO: 121 or SEQ ID NO: 122.

27. The guide RNA according to para 26, wherein from one to five of the terminal nucleotides at 5′ end and/or 3′ end of the guide RNA are chemically modified to enhance stability, optionally wherein three terminal nucleotides at 5′ end and/or 3′ end if the guide RNA are chemically modified to enhance stability, optionally wherein the chemical modification is modification with 2′-O-methyl 3′phosphorothioate.

28. A kit, a composition, or a gene-editing system, comprising the polynucleotide according to any one of paras 1 to 23 or the vector according to any one of paras 24 or 25.

29. The kit, composition, gene-editing system according to para 28, wherein the kit, composition, or gene-editing system further comprises a guide RNA according to para 26 or para 27.

30. The kit, composition, or gene-editing system, according to para 28 or para 29, wherein the kit, composition, or gene-editing system, further comprises a RNA-guided nuclease, optionally wherein the RNA-guided nuclease is a Cas9 endonuclease.

31. Use of the isolated polynucleotide according to any one of paras 1 to 23, the vector according to any one of paras 24 or 25, the guide RNA according to any one of paras 26 or 27, or the kit, composition, or gene-editing system according to any one of paras 28 to 30, for gene editing a cell or a population of cells.

32. An isolated genome comprising the polynucleotide according to any one of paras 1 to 23.

33. An isolated cell comprising the polynucleotide according to any one of paras 1 to 23 or the genome according to para 32.

34. The isolated cell according to para 33, wherein the cell is a hematopoietic stem cell (HSC), a hematopoietic progenitor cell (HPC), or a lymphoid progenitor cell (LPC).

35. The isolated cell according to para 33 or para 34, wherein the cell is a CD34⁺ cell.

36. A population of cells comprising one or more isolated cells according to any one of paras 33 to 35.

37. The population of cells according to para 36, wherein at least 50% of the population of cells are CD34⁺ cells.

38. The population of cells according to para 36 or para 37, wherein at least 20% of the population of cells are CD34⁺ cells comprising the genome according to para 25.

39. A method of gene editing a population of cells comprising:
(a) providing a population of cells; and
(b) delivering an RNA-guided nuclease, a guide RNA according to para 26 or para 27, and a vector according to para 24 or para 25, to the population of cells to obtain a population of gene-edited cells.

40. A method of treating a RAG-deficient immunodeficiency in a subject comprising: (a) providing a population of cells;
(b) delivering an RNA-guided nuclease, a guide RNA according to para 26 or para 27, and a vector according to para 24 or para 25, to the population of cells to obtain a population of gene-edited cells.
(c) administering the population of gene-edited cells to the subject.

41. The method according to para 39 or para 40, wherein the population of cells comprises or consists of HSCs, HPCs, and/or LPCs and/or wherein the population of cells comprises or consists of CD34⁺ cells.

42. The method according to any one of paras 39 to 41, wherein the population of cells is pre-activated, optionally wherein the population of cells is cultured with one or more cytokines selected from: one or more early acting cytokines such as TPO, IL-6, IL-3, SCF, FLT3-L; one or more transduction enhancers such as PGE2; and one or more expansion enhancers such as UM171, UM729, SR1.

43. The method according to any one of paras 39 to 42, wherein the RNA-guided nuclease and/or guide RNA is delivered prior to the vector and/or simultaneously with the vector.

44. The method according to any one of paras 39 to 43, wherein the RNA-guided nuclease is Cas9, optionally wherein the Cas9 and the guide RNA are delivered preassembled as Cas9 RNPs.

45. The method according to any one of paras 39 to 44, wherein the method further comprises delivering a p53 inhibitor and/or a HDR enhancer, optionally wherein the p53 inhibitor and/or a HDR enhancer is delivered simultaneously with the RNA-guided nuclease and/or guide RNA.

46. The method according to any one of paras 39 to 45, wherein the population of gene-edited cells is defined according to any one of paras 36 to 38.

47. A population of gene-edited cells obtainable by the method according to any one of paras 39 to 46.

48. A method of treating a RAG-deficient immunodeficiency comprising administering the isolated cell according to any one of paras 33 to 35, the population of cells according to any one of paras 36 to 38, or the population of gene-edited cells according to para 47, to a subject in need thereof.

49. The isolated cell according to any one of paras 33 to 35, the population of cells according to any one of paras 36 to 38, or the population of gene-edited cells according to para 47, for use in treating a RAG-deficient immunodeficiency in a subject.

50. The method according to para 48, or the isolated cell, population of cells, or population of gene-edited cells for use according to para 49, wherein the RAG-deficient immunodeficiency is T- B-severe combined immunodeficiency (SCID), Omenn syndrome, atypical SCID or combined immunodeficiency with granuloma/autoimmunity (CID-G/AI).

51. The method according to para 48 or para 50, or the isolated cell, population of cells, or population of gene-edited cells for use according to para 49 or para 50, wherein the subject has a RAG1 deficiency.

52. The method according to any one of paras 48, 50, or 51, or the isolated cell, population of cells, or population of gene-edited cells for use according to any one of paras 49 to 51, wherein the subject has a mutation in the RAG1 gene, optionally in RAG1 exon 2.

### Other Embodiments

Various features and embodiments of the present invention will now be described with reference to the following numbered paragraphs (paras).

1. An isolated polynucleotide comprising from 5′ to 3′: a first homology region, a nucleotide sequence encoding a RAG1 polypeptide fragment, and a second homology region, wherein the first homology region is homologous to a first region of the RAG1 exon 2 and the second homology region is homologous to a second region of the RAG1 exon 2.

2. The isolated polynucleotide according to para 1, wherein:
(i) the first homology region is homologous to a region upstream of chr 11:36574368 and the second homology region is homologous to a region downstream of chr 11:36574369;
(ii) the first homology region is homologous to a region upstream of chr 11:36574367 and the second homology region is homologous to a region downstream of chr 11:36574368;
(iii) the first homology region is homologous to a region upstream of chr 11:36574394 and the second homology region is homologous to a region downstream of chr 11:36574395;
(iv) the first homology region is homologous to a region upstream of chr 11:36574294 and the second homology region is homologous to a region downstream of chr 11:36574295;
(v) the first homology region is homologous to a region upstream of chr 11:36574109 and the second homology region is homologous to a region downstream of chr 11:36574110;
(vi) the first homology region is homologous to a region upstream of chr 11:36573910 and the second homology region is homologous to a region downstream of chr 11:36573911;
(vii) the first homology region is homologous to a region upstream of chr 11:36573878 and the second homology region is homologous to a region downstream of chr 11:36573879;
(viii) the first homology region is homologous to a region upstream of chr 11:36573959 and the second homology region is homologous to a region downstream of chr 11:36573960;
(ix) the first homology region is homologous to a region upstream of chr 11:36573957 and the second homology region is homologous to a region downstream of chr 11:36573958;
(x) the first homology region is homologous to a region upstream of chr 11:36573879 and the second homology region is homologous to a region downstream of chr 11:36573880;
(xi) the first homology region is homologous to a region upstream of chr 11:36573892 and the second homology region is homologous to a region downstream of chr 11:36573893;
(xii) the first homology region is homologous to a region upstream of chr 11:36573955 and the second homology region is homologous to a region downstream of chr 11:36573956;
(xiii) the first homology region is homologous to a region upstream of chr 11:36573878 and the second homology region is homologous to a region downstream of chr 11:36573879; or
(xiv) the first homology region is homologous to a region upstream of chr 11:36574406 and the second homology region is homologous to a region downstream of chr 11:36574407.

3. The isolated polynucleotide according to para 1 or 2, wherein:
(v) the first homology region is homologous to a region upstream of chr 11:36574109 and the second homology region is homologous to a region downstream of chr 11:36574110; or
(vi) the first homology region is homologous to a region upstream of chr 11:36573910 and the second homology region is homologous to a region downstream of chr 11:36573911.

4. The isolated polynucleotide according to para 1 or 2, wherein:
(xi) the first homology region is homologous to a region upstream of chr 11:36573892 and the second homology region is homologous to a region downstream of chr 11:36573893; or
(xiii) the first homology region is homologous to a region upstream of chr 11:36573878 and the second homology region is homologous to a region downstream of chr 11:36573879.

5. The isolated polynucleotide according to any preceding para, wherein:
(i) the first homology region is homologous to a region comprising chr 11:36574319-36574368 and/or the second homology region is homologous to a region comprising chr 11:36574369-36574418;
(ii) the first homology region is homologous to a region comprising chr 11:36574318-36574367 and/or the second homology region is homologous to a region comprising chr 11:36574368-36574417;
(iii) the first homology region is homologous to a region comprising chr 11:36574345-36574394 and/or the second homology region is homologous to a region comprising chr 11:36574395-36574444;
(iv) the first homology region is homologous to a region comprising chr 11:36574245-36574294 and/or the second homology region is homologous to a region comprising chr 11:36574295-36574344;
(v) the first homology region is homologous to a region comprising chr 11:36574060-36574109 and/or the second homology region is homologous to a region comprising chr 11:36574110-36574159;
(vi) the first homology region is homologous to a region comprising chr 11:36573861-36573910 and/or the second homology region is homologous to a region comprising chr 11:36573911-36573960;
(vii) the first homology region is homologous to a region comprising chr 11:36573829-36573878 and/or the second homology region is homologous to a region comprising chr 11:36573879-36573928;
(viii) the first homology region is homologous to a region comprising chr 11:36573910-36573959 and/or the second homology region is homologous to a region comprising chr 11:36573960-36574009;
(ix) the first homology region is homologous to a region comprising chr 11:36573908-36573957 and/or the second homology region is homologous to a region comprising chr 11:36573958-36574007;
(x) the first homology region is homologous to a region comprising chr 11:36573830-36573879 and/or the second homology region is homologous to a region comprising chr 11:36573880-36573929;
(xi) the first homology region is homologous to a region comprising chr 11:36573843-36573892 and/or the second homology region is homologous to a region comprising chr 11:36573893-36573942;
(xii) the first homology region is homologous to a region comprising chr 11:36573906-36573955 and/or the second homology region is homologous to a region comprising chr 11:36573956-36574005;
(xiii) the first homology region is homologous to a region comprising chr 11:36573829-36573878 and/or the second homology region is homologous to a region comprising chr 11:36573879-36573928; or
(xiv) the first homology region is homologous to a region comprising chr 11:36574357-36574406 and/or the second homology region is homologous to a region comprising chr 11:36574407-36574456.

6. The isolated polynucleotide according to any preceding para, wherein:
(i) the 3′ terminal sequence of the first homology region comprises or consists of a nucleotide sequence that has at least 70% identity to SEQ ID NO: 25 and/or the 5′ terminal sequence of the second homology region comprises or consists of a nucleotide sequence that has at least 70% identity to SEQ ID NO: 45;
(ii) the 3′ terminal sequence of the first homology region comprises or consists of a nucleotide sequence that has at least 70% identity to SEQ ID NO: 26 and/or the 5′ terminal sequence of the second homology region comprises or consists of a nucleotide sequence that has at least 70% identity to SEQ ID NO: 46;
(iii) the 3′ terminal sequence of the first homology region comprises or consists of a nucleotide sequence that has at least 70% identity to SEQ ID NO: 27 and/or the 5′ terminal sequence of the second homology region comprises or consists of a nucleotide sequence that has at least 70% identity to SEQ ID NO: 47;
(iv) the 3′ terminal sequence of the first homology region comprises or consists of a nucleotide sequence that has at least 70% identity to SEQ ID NO: 28 and/or the 5′ terminal sequence of the second homology region comprises or consists of a nucleotide sequence that has at least 70% identity to SEQ ID NO: 48;
(v) the 3′ terminal sequence of the first homology region comprises or consists of a nucleotide sequence that has at least 70% identity to SEQ ID NO: 29 or SEQ ID NO: 39 and/or the 5′ terminal sequence of the second homology region comprises or consists of a nucleotide sequence that has at least 70% identity to SEQ ID NO: 49 or SEQ ID NO: 59;
(vi) the 3′ terminal sequence of the first homology region comprises or consists of a nucleotide sequence that has at least 70% identity to SEQ ID NO: 30 or SEQ ID NO: 40 and/or the 5′ terminal sequence of the second homology region comprises or consists of a nucleotide sequence that has at least 70% identity to SEQ ID NO: 50 or SEQ ID NO: 60;
(vii) the 3′ terminal sequence of the first homology region comprises or consists of a nucleotide sequence that has at least 70% identity to SEQ ID NO: 31 or SEQ ID NO: 41 and/or the 5′ terminal sequence of the second homology region comprises or consists of a nucleotide sequence that has at least 70% identity to SEQ ID NO: 51;
(viii) the 3′ terminal sequence of the first homology region comprises or consists of a nucleotide sequence that has at least 70% identity to SEQ ID NO: 32 or SEQ ID NO: 42 and/or the 5′ terminal sequence of the second homology region comprises or consists of a nucleotide sequence that has at least 70% identity to SEQ ID NO: 52;
(ix) the 3′ terminal sequence of the first homology region comprises or consists of a nucleotide sequence that has at least 70% identity to SEQ ID NO: 33 or SEQ ID NO: 42 and/or the 5′ terminal sequence of the second homology region comprises or consists of a nucleotide sequence that has at least 70% identity to SEQ ID NO: 53;
(x) the 3′ terminal sequence of the first homology region comprises or consists of a nucleotide sequence that has at least 70% identity to SEQ ID NO: 34 or SEQ ID NO: 41 and/or the 5′ terminal sequence of the second homology region comprises or consists of a nucleotide sequence that has at least 70% identity to SEQ ID NO: 54;
(xi) the 3′ terminal sequence of the first homology region comprises or consists of a nucleotide sequence that has at least 70% identity to SEQ ID NO: 35 or SEQ ID NO: 41 and/or the 5′ terminal sequence of the second homology region comprises or consists of a nucleotide sequence that has at least 70% identity to SEQ ID NO: 55;
(xii) the 3′ terminal sequence of the first homology region comprises or consists of a nucleotide sequence that has at least 70% identity to SEQ ID NO: 36 or SEQ ID NO: 42 and/or the 5′ terminal sequence of the second homology region comprises or consists of a nucleotide sequence that has at least 70% identity to SEQ ID NO: 56;
(xiii) the 3′ terminal sequence of the first homology region comprises or consists of a nucleotide sequence that has at least 70% identity to SEQ ID NO: 37 or SEQ ID NO: 43 and/or the 5′ terminal sequence of the second homology region comprises or consists of a nucleotide sequence that has at least 70% identity to SEQ ID NO: 57; or
(xiv) the 3′ terminal sequence of the first homology region comprises or consists of a nucleotide sequence that has at least 70% identity to SEQ ID NO: 38 or SEQ ID NO: 44 and/or the 5′ terminal sequence of the second homology region comprises or consists of a nucleotide sequence that has at least 70% identity to SEQ ID NO: 58.

7. The isolated polynucleotide according to any preceding para, wherein:
(1) the first homology region comprises or consists of a nucleotide sequence that has at least 70% identity to SEQ ID NO: 69, or a fragment thereof and/or the second homology region comprises or consists of a nucleotide sequence that has at least 70% identity to SEQ ID NO: 77, or a fragment thereof;
(4) the first homology region comprises or consists of a nucleotide sequence that has at least 70% identity to SEQ ID NO: 71, or a fragment thereof and/or the second homology region comprises or consists of a nucleotide sequence that has at least 70% identity to SEQ ID NO: 78, or a fragment thereof;
(5) the first homology region comprises or consists of a nucleotide sequence that has at least 70% identity to SEQ ID NO: 72, or a fragment thereof and/or the second homology region comprises or consists of a nucleotide sequence that has at least 70% identity to SEQ ID NO: 79, or a fragment thereof; or
(6) the first homology region comprises or consists of a nucleotide sequence that has at least 70% identity to SEQ ID NO: 72, or a fragment thereof and/or the second homology region comprises or consists of a nucleotide sequence that has at least 70% identity to SEQ ID NO: 80, or a fragment thereof.

8. The isolated polynucleotide according to any of paras 1 to 6, wherein:
(12) the first homology region comprises or consists of a nucleotide sequence that has at least 70% identity to SEQ ID NO: 153, or a fragment thereof and the second homology region comprises or consists of a nucleotide sequence that has at least 70% identity to SEQ ID NO: 155, or a fragment thereof;
(13) the first homology region comprises or consists of a nucleotide sequence that has at least 70% identity to SEQ ID NO: 153, or a fragment thereof and the second homology region comprises or consists of a nucleotide sequence that has at least 70% identity to SEQ ID NO: 157, or a fragment thereof;
(14) the first homology region comprises or consists of a nucleotide sequence that has at least 70% identity to SEQ ID NO: 154, or a fragment thereof and the second homology region comprises or consists of a nucleotide sequence that has at least 70% identity to SEQ ID NO: 156, or a fragment thereof; or
(15) the first homology region comprises or consists of a nucleotide sequence that has at least 70% identity to SEQ ID NO: 154, or a fragment thereof and the second homology region comprises or consists of a nucleotide sequence that has at least 70% identity to SEQ ID NO: 157, or a fragment thereof.

9. The isolated polynucleotide according to any preceding para, wherein the first and second homology regions are each 50-2000 bp in length, 50-1800 bp in length, 50-1500 bp in length, 50-1000 bp in length, 100-500 bp in length, or 200-400 bp in length.

10. An isolated polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a first region of the RAG1 intron 1 or exon 2 and the second homology region is homologous to a second region of the RAG1 exon 2.

11. The isolated polynucleotide according to para 10, wherein the first homology region is homologous to a region upstream of: (i) chr 11:36569295; (ii) chr 11:36573790; (iii) chr 11:36573641; (iv) chr 11:36573351; (v) chr 11:36569080; (vi) chr 11:36572472; (vii) chr 11:36571458; (viii) chr 11:36571366; (ix) chr 11:36572859 (x) chr 11:36571457; (xi) chr 11:36569351; or (xii) chr 11:36572375, preferably wherein the first homology region is homologous to a region upstream of: (i) chr 11:36569295; (ii) chr 11:36573351; (iii) chr 11:36571366, more preferably wherein the first homology region is homologous to a region upstream of chr 11:36569295.

12. The isolated polynucleotide according to para 10 or 11, wherein the first homology region is homologous to a region comprising chr 11:36569245-chr 11:36569294, preferably wherein the 3′ terminal sequence of the first homology region comprises or consists of a nucleotide sequence that has at least 70% identity to SEQ ID NO: 81, more preferably wherein the first homology region comprises or consists of a nucleotide sequence that has at least 70% identity to SEQ ID NO: 93.

13. The isolated polynucleotide according to any preceding para, wherein the second homology region is homologous to a region downstream of chr 11:36574557; downstream of chr 11:36574870; downstream of chr 11:36575183; downstream of chr 11:36575496; downstream of chr 11:36575810; downstream of chr 11:36576123; or downstream of chr 11:36576436, preferably wherein the second homology region is homologous to a region comprising chr 11:36576437-chr 11:36576536.

14. The isolated polynucleotide according to any preceding para, wherein the second homology region comprises or consists of a nucleotide sequence that has at least 70% identity to any of SEQ ID NOs: 79-80, 94 or 157, or a fragment thereof, preferably wherein the 5′ terminal sequence of the second homology region comprises or consists of a nucleotide sequence that has at least 70% identity to SEQ ID NO: 67.

15. The isolated polynucleotide according to any of paras 1 to 9 or paras 13 or 14, wherein:
(2) the first homology region comprises or consists of a nucleotide sequence that has at least 70% identity to SEQ ID NO: 70, or a fragment thereof and/or the second homology region comprises or consists of a nucleotide sequence that has at least 70% identity to SEQ ID NO: 79, or a fragment thereof;
(3) the first homology region comprises or consists of a nucleotide sequence that has at least 70% identity to SEQ ID NO: 70, or a fragment thereof and/or the second homology region comprises or consists of a nucleotide sequence that has at least 70% identity to SEQ ID NO: 80, or a fragment thereof;
(7) the first homology region comprises or consists of a nucleotide sequence that has at least 70% identity to SEQ ID NO: 73, or a fragment thereof and/or the second homology region comprises or consists of a nucleotide sequence that has at least 70% identity to SEQ ID NO: 79, or a fragment thereof;
(8) the first homology region comprises or consists of a nucleotide sequence that has at least 70% identity to SEQ ID NO: 74, or a fragment thereof and/or the second homology region comprises or consists of a nucleotide sequence that has at least 70% identity to SEQ ID NO: 79, or a fragment thereof;
(9) the first homology region comprises or consists of a nucleotide sequence that has at least 70% identity to SEQ ID NO: 75, or a fragment thereof and/or the second homology region comprises or consists of a nucleotide sequence that has at least 70% identity to SEQ ID NO: 79, or a fragment thereof; or
(10) the first homology region comprises or consists of a nucleotide sequence that has at least 70% identity to SEQ ID NO: 76, or a fragment thereof and/or the second homology region comprises or consists of a nucleotide sequence that has at least 70% identity to SEQ ID NO: 79, or a fragment thereof.

16. The isolated polynucleotide according to any of paras 10 to 14, wherein:
(11) the first homology region comprises or consists of a nucleotide sequence that has at least 70% identity to SEQ ID NO: 93, or a fragment thereof and/or the second homology region comprises or consists of a nucleotide sequence that has at least 70% identity to SEQ ID NO: 94, or a fragment thereof.

17. The isolated polynucleotide according to any preceding para, wherein the first homology region is about 50-1000 bp in length, 100-500 bp in length, or 200-400 bp in length; and/or wherein the second homology region is about 500-2000 bp in length, 1000-2000 bp in length, or 1500-2000 bp in length.

18. The isolated polynucleotide according to any preceding para, wherein the nucleotide sequence encoding a RAG1 polypeptide fragment comprises or consists of a nucleotide sequence encoding a fragment of an amino acid sequence that has at least 70% identity to SEQ ID NO: 4, SEQ ID NO: 5, or SEQ ID NO: 6.

19. The isolated polynucleotide according to any preceding para, wherein the RAG1 polypeptide fragment is at least 500 amino acids in length, at least 550 amino acids in length, at least 600 amino acids in length, at least 650 amino acids in length, at least 700 amino acids in length, at least 750 amino acids in length, or at least 800 amino acids in length.

20. The isolated polynucleotide according to any preceding para, wherein the RAG1 polypeptide fragment comprises or consists of an amino acid sequence that has at least 70% identity to any one of SEQ ID NOs: 7 to 14, 164 or 165.

21. The isolated polynucleotide according to any preceding para, wherein the nucleotide sequence encoding a RAG1 polypeptide fragment comprises or consists of a fragment of a nucleotide sequence that has at least 70% identity to SEQ ID NO: 15.

22. The isolated polynucleotide according to any preceding para, wherein the nucleotide sequence encoding a RAG1 polypeptide fragment comprises or consists of a nucleotide sequence that has at least 70% identity to any one of SEQ ID NOs: 17 to 24, 158 or 159.

23. The isolated polynucleotide according to any of paras 10 to 14 or paras 16 to 22, wherein the splice acceptor site comprises or consists of a nucleotide sequence that has at least 70% identity to SEQ ID NO: 95.

24. The isolated polynucleotide according to para 1, wherein the polynucleotide comprises or consists of a nucleotide sequence that has at least 70% identity to any one of SEQ ID NOs: 106 to 115 or 160 to 163.

25. The isolated polynucleotide according to para 10, wherein the polynucleotide comprises or consists of a nucleotide sequence that has at least 70% identity to SEQ ID NO: 116.

26. A vector comprising the polynucleotide according to any preceding para.

27. The vector according to para 26, wherein the vector is a viral vector, optionally an adeno-associated viral (AAV) vector such as an AAV6 vector.

28. A guide RNA comprising or consisting of a nucleotide sequence that has at least 90% identity to any of SEQ ID NOs: 117-130, optionally wherein the guide RNA comprises or consists of a nucleotide sequence that has at least 90% identity to SEQ ID NO: 121 or SEQ ID NO: 122.

29. The guide RNA according to para 28, wherein from one to five of the terminal nucleotides at 5′ end and/or 3′ end of the guide RNA are chemically modified to enhance stability, optionally wherein three terminal nucleotides at 5′ end and/or 3′ end if the guide RNA are chemically modified to enhance stability, optionally wherein the chemical modification is modification with 2′-O-methyl 3′phosphorothioate.

30. A kit, a composition, or a gene-editing system, comprising the polynucleotide according to any one of paras 1 to 25 or the vector according to any one of paras 26 or 27.

31. The kit, composition, gene-editing system according to para 30, wherein the kit, composition, or gene-editing system further comprises a guide RNA according to para 28 or para 29.

32. The kit, composition, or gene-editing system, according to para 30 or para 31, wherein the kit, composition, or gene-editing system, further comprises a RNA-guided nuclease, optionally wherein the RNA-guided nuclease is a Cas9 endonuclease.

33. Use of the isolated polynucleotide according to any one of paras 1 to 25, the vector according to any one of paras 26 or 27, the guide RNA according to any one of paras 28 or 29, or the kit, composition, or gene-editing system according to any one of paras 30 to 32, for gene editing a cell or a population of cells.

34. An isolated genome comprising the polynucleotide according to any one of paras 1 to 25.

35. An isolated cell comprising the polynucleotide according to any one of paras 1 to 25 or the genome according to para 34.

36. The isolated cell according to para 35, wherein the cell is a hematopoietic stem cell (HSC), a hematopoietic progenitor cell (HPC), or a lymphoid progenitor cell (LPC).

37. The isolated cell according to para 35 or para 36, wherein the cell is a CD34⁺ cell.

38. A population of cells comprising one or more isolated cells according to any one of paras 35 to 37.

39. The population of cells according to para 38, wherein at least 50% of the population of cells are CD34⁺ cells.

40. The population of cells according to para 38 or para 39, wherein at least 20% of the population of cells are CD34⁺ cells comprising the genome according to para 27.

41. A method of gene editing a population of cells comprising:
(a) providing a population of cells; and
(b) delivering an RNA-guided nuclease, a guide RNA according to para 28 or para 29, and a vector according to para 26 or para 27, to the population of cells to obtain a population of gene-edited cells.

42. A method of treating a RAG-deficient immunodeficiency in a subject comprising:
(a) providing a population of cells;
(b) delivering an RNA-guided nuclease, a guide RNA according to para 28 or para 29, and a vector according to para 26 or para 27, to the population of cells to obtain a population of gene-edited cells.
(c) administering the population of gene-edited cells to the subject.

43. The method according to para 41 or para 42, wherein the population of cells comprises or consists of HSCs, HPCs, and/or LPCs and/or wherein the population of cells comprises or consists of CD34⁺ cells.

44. The method according to any one of paras 41 to 43, wherein the population of cells is pre-activated, optionally wherein the population of cells is cultured with one or more cytokines selected from: one or more early acting cytokines such as TPO, IL-6, IL-3, SCF, FLT3-L; one or more transduction enhancers such as PGE2; and one or more expansion enhancers such as UM171, UM729, SR1.

45. The method according to any one of paras 41 to 44, wherein the RNA-guided nuclease and/or guide RNA is delivered prior to the vector and/or simultaneously with the vector.

46. The method according to any one of paras 41 to 45, wherein the RNA-guided nuclease is Cas9, optionally wherein the Cas9 and the guide RNA are delivered preassembled as Cas9 RNPs.

47. The method according to any one of paras 41 to 46, wherein the method further comprises delivering a p53 inhibitor and/or a HDR enhancer, optionally wherein the p53 inhibitor and/or a HDR enhancer is delivered simultaneously with the RNA-guided nuclease and/or guide RNA.

48. The method according to any one of paras 41 to 47, wherein the population of gene-edited cells is defined according to any one of paras 38 to 40.

49. A population of gene-edited cells obtainable by the method according to any one of paras 41 to 48.

50. A method of treating a RAG-deficient immunodeficiency comprising administering the isolated cell according to any one of paras 35 to 37, the population of cells according to any one of paras 38 to 40, or the population of gene-edited cells according to para 49, to a subject in need thereof.

51. The isolated cell according to any one of paras 35 to 37, the population of cells according to any one of paras 38 to 40, or the population of gene-edited cells according to para 49, for use in treating a RAG-deficient immunodeficiency in a subject.

52. The method according to para 50, or the isolated cell, population of cells, or population of gene-edited cells for use according to para 51, wherein the RAG-deficient immunodeficiency is T- B-severe combined immunodeficiency (SCID), Omenn syndrome, atypical SCID or combined immunodeficiency with granuloma/autoimmunity (CID-G/AI).

53. The method according to para 50 or para 52, or the isolated cell, population of cells, or population of gene-edited cells for use according to para 51 or para 52, wherein the subject has a RAG1 deficiency.

54. The method according to any one of paras 50, 52, or 53, or the isolated cell, population of cells, or population of gene-edited cells for use according to any one of paras 51 to 53, wherein the subject has a mutation in the RAG1 gene, optionally in RAG1 exon 2.

1-55. (canceled)

56. An isolated polynucleotide comprising from 5′ to 3′: a first homology region, a nucleotide sequence encoding a RAG1 polypeptide fragment, and a second homology region, wherein the first homology region is homologous to a first region of the RAG1 exon 2 and the second homology region is homologous to a second region of the RAG1 exon 2.

57. The isolated polynucleotide according to claim 56, wherein:
(i) the first homology region is homologous to a region upstream of chr 11:36574368 and the second homology region is homologous to a region downstream of chr 11:36574369;
(ii) the first homology region is homologous to a region upstream of chr 11:36574367 and the second homology region is homologous to a region downstream of chr 11:36574368;
(iii) the first homology region is homologous to a region upstream of chr 11:36574394 and the second homology region is homologous to a region downstream of chr 11:36574395;
(iv) the first homology region is homologous to a region upstream of chr 11:36574294 and the second homology region is homologous to a region downstream of chr 11:36574295;
(v) the first homology region is homologous to a region upstream of chr 11:36574109 and the second homology region is homologous to a region downstream of chr 11:36574110;
(vi) the first homology region is homologous to a region upstream of chr 11:36573910 and the second homology region is homologous to a region downstream of chr 11:36573911;
(vii) the first homology region is homologous to a region upstream of chr 11:36573878 and the second homology region is homologous to a region downstream of chr 11:36573879;
(viii) the first homology region is homologous to a region upstream of chr 11:36573959 and the second homology region is homologous to a region downstream of chr 11:36573960;
(ix) the first homology region is homologous to a region upstream of chr 11:36573957 and the second homology region is homologous to a region downstream of chr 11:36573958;
(x) the first homology region is homologous to a region upstream of chr 11:36573879 and the second homology region is homologous to a region downstream of chr 11:36573880;
(xi) the first homology region is homologous to a region upstream of chr 11:36573892 and the second homology region is homologous to a region downstream of chr 11:36573893;
(xii) the first homology region is homologous to a region upstream of chr 11:36573955 and the second homology region is homologous to a region downstream of chr 11:36573956;
(xiii) the first homology region is homologous to a region upstream of chr 11:36573878 and the second homology region is homologous to a region downstream of chr 11:36573879; or
(xiv) the first homology region is homologous to a region upstream of chr 11:36574406 and the second homology region is homologous to a region downstream of chr 11:36574407.

58. The isolated polynucleotide according to claim 56, wherein:
(xi) the first homology region is homologous to a region upstream of chr 11:36573892 and the second homology region is homologous to a region downstream of chr 11:36573893; or
(xiii) the first homology region is homologous to a region upstream of chr 11:36573878 and the second homology region is homologous to a region downstream of chr 11:36573879.

59. The isolated polynucleotide according to claim 56, wherein the first homology region is homologous to a region upstream of chr 11:36573878 and the second homology region is homologous to a region downstream of chr 11:36573879.

60. The isolated polynucleotide according to claim 56, wherein:
(i) the first homology region is homologous to a region comprising chr 11: 36574319-36574368 and/or the second homology region is homologous to a region comprising chr 11:36574369-36574418;
(ii) the first homology region is homologous to a region comprising chr 11: 36574318-36574367 and/or the second homology region is homologous to a region comprising chr 11:36574368-36574417;
(iii) the first homology region is homologous to a region comprising chr 11: 36574345-36574394 and/or the second homology region is homologous to a region comprising chr 11:36574395-36574444;
(iv) the first homology region is homologous to a region comprising chr 11: 36574245-36574294 and/or the second homology region is homologous to a region comprising chr 11:36574295-36574344;
(v) the first homology region is homologous to a region comprising chr 11: 36574060-36574109 and/or the second homology region is homologous to a region comprising chr 11:36574110-36574159;
(vi) the first homology region is homologous to a region comprising chr 11: 36573861-36573910 and/or the second homology region is homologous to a region comprising chr 11:3657391 1-36573960;
(vii) the first homology region is homologous to a region comprising chr 11: 36573829-36573878 and/or the second homology region is homologous to a region comprising chr 11:36573879-36573928;
(viii) the first homology region is homologous to a region comprising chr 11:36573910-36573959 and/or the second homology region is homologous to a region comprising chr 11:36573960-36574009;
(ix) the first homology region is homologous to a region comprising chr 11: 36573908-36573957 and/or the second homology region is homologous to a region comprising chr 11:36573958-36574007;
(x) the first homology region is homologous to a region comprising chr 11: 36573830-36573879 and/or the second homology region is homologous to a region comprising chr 11:36573880-36573929;
(xi) the first homology region is homologous to a region comprising chr 11: 36573843-36573892 and/or the second homology region is homologous to a region comprising chr 11:36573893-36573942;
(xii) the first homology region is homologous to a region comprising chr 11: 36573906-36573955 and/or the second homology region is homologous to a region comprising chr 11:36573956-36574005;
(xiii) the first homology region is homologous to a region comprising chr 11:36573829-36573878 and/or the second homology region is homologous to a region comprising chr 11:36573879-36573928; or
(xiv) the first homology region is homologous to a region comprising chr 11: 36574357-36574406 and/or the second homology region is homologous to a region comprising chr 11:36574407-36574456.

61. The isolated polynucleotide according to claim 56, wherein:
(i) the 3′ terminal sequence of the first homology region comprises a nucleotide sequence that has at least 70% identity to SEQ ID NO: 25 and/or the 5′ terminal sequence of the second homology region comprises a nucleotide sequence that has at least 70% identity to SEQ ID NO: 45;
(ii) the 3′ terminal sequence of the first homology region comprises a nucleotide sequence that has at least 70% identity to SEQ ID NO: 26 and/or the 5′ terminal sequence of the second homology region comprises a nucleotide sequence that has at least 70% identity to SEQ ID NO: 46;
(iii) the 3′ terminal sequence of the first homology region comprises a nucleotide sequence that has at least 70% identity to SEQ ID NO: 27 and/or the 5′ terminal sequence of the second homology region comprises a nucleotide sequence that has at least 70% identity to SEQ ID NO: 47;
(iv) the 3′ terminal sequence of the first homology region comprises a nucleotide sequence that has at least 70% identity to SEQ ID NO: 28 and/or the 5′ terminal sequence of the second homology region comprises a nucleotide sequence that has at least 70% identity to SEQ ID NO: 48;
(v) the 3′ terminal sequence of the first homology region comprises a nucleotide sequence that has at least 70% identity to SEQ ID NO: 29 or SEQ ID NO:
39 and/or the 5′ terminal sequence of the second homology region comprises a nucleotide sequence that has at least 70% identity to SEQ ID NO: 49 or SEQ ID NO: 59;
(vi) the 3′ terminal sequence of the first homology region comprises a nucleotide sequence that has at least 70% identity to SEQ ID NO: 30 or SEQ ID NO:
40 and/or the 5′ terminal sequence of the second homology region comprises a nucleotide sequence that has at least 70% identity to SEQ ID NO: 50 or SEQ ID NO: 60;
(vii) the 3′ terminal sequence of the first homology region comprises a nucleotide sequence that has at least 70% identity to SEQ ID NO: 31 or SEQ ID NO:
41 and/or the 5′ terminal sequence of the second homology region comprises a nucleotide sequence that has at least 70% identity to SEQ ID NO: 51;
(viii) the 3′ terminal sequence of the first homology region comprises a nucleotide sequence that has at least 70% identity to SEQ ID NO: 32 or SEQ ID NO:
42 and/or the 5′ terminal sequence of the second homology region comprises a nucleotide sequence that has at least 70% identity to SEQ ID NO: 52;
(ix) the 3′ terminal sequence of the first homology region comprises a nucleotide sequence that has at least 70% identity to SEQ ID NO: 33 or SEQ ID NO: 42 and/or the 5′ terminal sequence of the second homology region comprises a nucleotide sequence that has at least 70% identity to SEQ ID NO: 53;
(x) the 3′ terminal sequence of the first homology region comprises a nucleotide sequence that has at least 70% identity to SEQ ID NO: 34 or SEQ ID NO: 41 and/or the 5′ terminal sequence of the second homology region comprises a nucleotide sequence that has at least 70% identity to SEQ ID NO: 54;
(xi) the 3′ terminal sequence of the first homology region comprises a nucleotide sequence that has at least 70% identity to SEQ ID NO: 35 or SEQ ID NO:
41 and/or the 5′ terminal sequence of the second homology region comprises a nucleotide sequence that has at least 70% identity to SEQ ID NO: 55;
(xii) the 3′ terminal sequence of the first homology region comprises a nucleotide sequence that has at least 70% identity to SEQ ID NO: 36 or SEQ ID NO:
42 and/or the 5′ terminal sequence of the second homology region comprises a nucleotide sequence that has at least 70% identity to SEQ ID NO: 56; (xiii) the 3′ terminal sequence of the first homology region comprises a nucleotide sequence that has at least 70% identity to SEQ ID NO: 37 or SEQ ID NO:
43 and/or the 5′ terminal sequence of the second homology region comprises a nucleotide sequence that has at least 70% identity to SEQ ID NO: 57; or
(xiv) the 3′ terminal sequence of the first homology region comprises a nucleotide sequence that has at least 70% identity to SEQ ID NO: 38 or SEQ ID NO:
44 and/or the 5′ terminal sequence of the second homology region comprises a nucleotide sequence that has at least 70% identity to SEQ ID NO: 58.

62. The isolated polynucleotide according to claim 56, wherein:
(12) the first homology region comprises a nucleotide sequence that has at least 70% identity to SEQ ID NO: 153, or a fragment thereof and the second homology region comprises a nucleotide sequence that has at least 70% identity to SEQ ID NO: 155, or a fragment thereof,
(13) the first homology region comprises a nucleotide sequence that has at least 70% identity to SEQ ID NO: 153, or a fragment thereof and the second homology region comprises a nucleotide sequence that has at least 70% identity to SEQ ID NO: 157, or a fragment thereof,
(14) the first homology region comprises of a nucleotide sequence that has at least 70% identity to SEQ ID NO: 154, or a fragment thereof and the second homology region comprises a nucleotide sequence that has at least 70% identity to SEQ ID NO: 156, or a fragment thereof, or
(15) the first homology region comprises a nucleotide sequence that has at least 70% identity to SEQ ID NO: 154, or a fragment thereof and the second homology region comprises a nucleotide sequence that has at least 70% identity to SEQ ID NO: 157, or a fragment thereof.

63. An isolated polynucleotide comprising from 5′ to 3′: a first homology region, a splice acceptor sequence, a nucleotide sequence encoding a RAG1 polypeptide or a RAG1 polypeptide fragment, and a second homology region, wherein the first homology region is homologous to a first region of the RAG1 intron 1 or exon 2 and the second homology region is homologous to a second region of the RAG1 exon 2.

64. The isolated polynucleotide according to claim 63, wherein the first homology region is homologous to a region upstream of: (i) chr 11:36569295; (ii) chr 11:36573790; (iii) chr 11:36573641; (iv) chr 11:36573351; (v) chr 11:36569080; (vi) chr 11:36572472; (vii) chr 11:36571458; (viii) chr 11:36571366; (ix) chr 11:36572859 (x) chr 11:36571457; (xi) chr 11:36569351; or (xii) chr 11:36572375, preferably wherein the first homology region is homologous to a region upstream of: (i) chr 11:36569295; (ii) chr 11:36573351; (iii) chr 11:36571366, more preferably wherein the first homology region is homologous to a region upstream of chr 11:36569295.

65. The isolated polynucleotide according to claim 63, wherein the first homology region is homologous to a region comprising chr 11:36569245-chr 11:36569294, preferably wherein the 3′ terminal sequence of the first homology region comprises a nucleotide sequence that has at least 70% identity to SEQ ID NO: 81, more preferably wherein the first homology region comprises a nucleotide sequence that has at least 70% identity to SEQ ID NO: 93.

66. The isolated polynucleotide according to claim 56, wherein the second homology region is homologous to a region downstream of chr 11:36574557; downstream of chr 11:36574870; downstream of chr 11:36575183; downstream of chr 11:36575496; downstream of chr 11:36575810; downstream of chr 11:36576123; or downstream of chr 11:36576436, preferably wherein the second homology region is homologous to a region comprising chr 11:36576437-chr 11:36576536.

67. The isolated polynucleotide according to claim 63, wherein the second homology region is homologous to a region downstream of chr 11:36574557; downstream of chr 11:36574870; downstream of chr 11:36575183; downstream of chr 11:36575496; downstream of chr 11:36575810; downstream of chr 11:36576123; or downstream of chr 11:36576436, preferably wherein the second homology region is homologous to a region comprising chr 11:36576437-chr 11:36576536.

68. The isolated polynucleotide according to claim 56, wherein the second homology region comprises a nucleotide sequence that has at least 70% identity to any of SEQ ID NOs: 79-80, 94 or 157, or a fragment thereof, preferably wherein the 5′ terminal sequence of the second homology region comprises a nucleotide sequence that has at least 70% identity to SEQ ID NO: 67.

69. The isolated polynucleotide according to claim 63, wherein the second homology region comprises a nucleotide sequence that has at least 70% identity to any of SEQ ID NOs: 79-80, 94 or 157, or a fragment thereof, preferably wherein the 5′ terminal sequence of the second homology region comprises a nucleotide sequence that has at least 70% identity to SEQ ID NO: 67.

70. A guide RNA comprising a nucleotide sequence that has at least 90% identity to any of SEQ ID NOs: 1 17-130.

71. The guide RNA according to claim 70, wherein the guide RNA comprises a nucleotide sequence that has at least 90% identity to SEQ ID NO: 127 or SEQ ID NO: 129, optionally wherein the guide RNA comprises a nucleotide sequence that has at least 90% identity to SEQ ID NO: 129.

72. The guide RNA according to claim 70, wherein from one to five of the terminal nucleotides at 5′ end and/or 3′ end of the guide RNA are chemically modified to enhance stability, optionally wherein three terminal nucleotides at 5′ end and/or 3′ end if the guide RNA are chemically modified to enhance stability, optionally wherein the chemical modification is modification with 2′-O-methyl 3′phosphorothioate.

73. A population of cells comprising one or more isolated cells comprising the polynucleotide according to claim 56.

74. A method of gene editing a population of cells comprising:
(a) providing a population of cells; and
(b) delivering an RNA-guided nuclease, a guide RNA comprising a nucleotide sequence that has at least 90% identity to any SEQ ID Nos 117-130, and a vector comprising the polynucleotide according to claim 56, to the population of cells to obtain a population of gene-edited cells.

75. A method of treating a RAG-deficient immunodeficiency in a subject comprising:
(a) providing a population of cells;
(b) delivering an RNA-guided nuclease, a guide RNA comprising a nucleotide sequence that has at least 90% identity to any SEQ ID Nos 117-130, and a vector, and a vector comprising the polynucleotide according to claim 56, to the population of cells to obtain a population of gene-edited cells.
(c) administering the population of gene-edited cells to the subject.
